(19) United States
(12) Patent Application Publication
(10) Pub. No.: US 2021/0380675 A1
(43) Pub. Date: Dec. 9, 2021

(54) IL-36 ANTIBODIES AND USES THEREOF

(71) Applicant: KYOWA KIRIN CO., LTD., Tokyo (JP)

(72) Inventors: Kacey Layn SACHEN, La Jolla, CA (US); Andrew John MCKNIGHT, San Diego, CA (US); Rachel Soloff NUGENT, San Diego, CA (US); John Lorca LAUDENSLAGER, La Mesa, CA (US); Giuseppe DESTITO, Encinitas, CA (US); Dirk Michael ZAJONC, La Jolla, CA (US); Aruna BITRA, La Jolla, CA (US); Takenao YAMADA, Tokyo (JP)

(73) Assignee: KYOWA KIRIN CO., LTD., Tokyo (JP)

(21) Appl. No.: 17/280,641

(22) PCT Filed: Sep. 27, 2019
(86) PCT No.: PCT/IB2019/058203

§ 371 Date: Mar. 26, 2021

Related U.S. Application Data
(60) Provisional application No. 62/739,074, filed on Sep. 28, 2018.

(51) Int. Cl. C07K 16/24

(52) U.S. Cl. CPC C07K 16/244; C07K 2317/76; C07K 2317/565; C07K 2317/33; C07K 2317/24; C07K 2317/10; C07K 2317/92

(57) ABSTRACT

An antibody or antigen binding fragment thereof that binds to an IL-36, wherein the antibody or antigen binding fragment thereof binds to both IL-36α and IL-36γ, and wherein the antibody is an antagonist of both IL-36α and IL-36γ.

### CROSS-REFERENCE TO RELATED APPLICATIONS

This application claims priority to U.S. provisional application No. 62/739,074 filed Sep. 28, 2018, which is incorporated herein by reference in its entirety.

### SEQUENCE LISTING

The present specification is being filed with a computer readable form (CRF) copy of the Sequence Listing. The CRF entitled 14233-005-228_SEQ_LISTING.TXT, which was created on Sep. 16, 2019 and is 162,628 bytes in size, also serves as the paper copy of the Sequence Listing and is incorporated herein by reference in its entirety.

### 1. FIELD

Provided herein are anti-IL-36 antibodies and pharmaceutical compositions, methods, and uses thereof.

### 2. BACKGROUND

Cytokines are involved in various biological processes such as immunological responses including but not limited to inflammatory responses, viral immunity, intracellular parasitic immunity, allograft rejection, humoral responses, helminth immunity and allergic response.

The interleukin 36 (IL-36) cytokine family is comprised of IL-36 Receptor antagonist (IL-36Ra) known as a natural antagonist, IL-36α, IL-36β, and IL-36γ (see Dinarello, C., et al., Nat Immunol, 2010, 11(11): 973). The IL-36 family of cytokines and their receptor have been implicated in numerous inflammatory conditions and diseases. For example, increased expression of IL-36 (particularly IL-36γ and IL-36α) have been detected in lesional skin from patients with generalized pustular psoriasis (GPP), as well as in other types of psoriasis such as plaque psoriasis, palmoplantar pustular psoriasis, and palmoplantar pustulosis (see Liang, Y., et al., J Allergy Clin Immunol, 2017. 139(4): 1217-1227; Bissonnette, R., et al., PLoS One, 2016. 11(5): e0155215; Johnston, A., et al., J Allergy Clin Immunol, 2017, 140(1): 109-120; D'Erme, A. M., et al., J Invest Dermatol, 2015, 135(4): 1025-1032; and Carrier, Y., et al., J Invest Dermatol, 2011. 131(12): 2428-37). Increased levels of IL-36γ can also be detected in lesional skin from patients with discoid lupus erythematosus and subacute cutaneous lupus erythematosus (see D'Erme, A. M., et al., J Invest Dermatol, 2015. 135(4): 1025-1032; and Jabbari, A., et al., J Invest Dermatol, 2014. 134(1): 87-95). For another example, elevated expression of IL-36α and IL-36γ has been measured from involved tissues from patients with inflammatory bowel disease including Crohn's disease and ulcerative colitis (see Russell, S. E., et al., Mucosal Immunol, 2016, 9(5): 1193-204; Nishida, A., et al., Inflamm Bowel Dis, 2016. 22(2): 303-14; and Boutet, M. A., et al., Clin Exp Immunol, 2016, 184(2): p. 159-73).

### 3. SUMMARY

In one aspect, provided herein is an antibody or antigen binding fragment thereof that binds to an IL-36, wherein the antibody or antigen binding fragment thereof binds to both IL-36α and IL-36γ, and wherein the antibody is an antagonist of both IL-36α and IL-36γ.

In some embodiments, the antibody or antigen binding fragment thereof simultaneously antagonizes both IL-36α and IL-36γ.

In some embodiments, the antibody or antigen binding fragment thereof provided herein binds to one or more amino acid residues selected from 45th amino acid residue to 100th amino acid residue of the amino acid sequence of IL-36α represented by SEQ ID NO: 5 or SEQ ID NO: 7 and/or the amino acid sequence of IL-36γ represented by SEQ ID NO: 10. In some embodiments, the antibody or antigen binding fragment thereof provided herein binds to one or more amino acid residues selected from Arg 45, His 46, Glu 48, Thr 49, Leu 50, Lys 85, Asp 89, Asn 92, Gln 93, Pro 94, Glu 95, Pro 96, Val 97, Lys 98 and Phe 100 of the amino acid sequence of IL-36α represented by SEQ ID NO: 5 or SEQ ID NO: 7 and/or one or more amino acid residues selected from Tyr 46, Glu 48, Ala 49, Leu 50, Gln 85, Gly 92, Gln 93, Pro 94, Glu 95, Pro 96, Val 97, Lys 98 and Phe 100 of the amino acid sequence of IL-36γ represented by SEQ ID NO: 10. In some embodiments, the antibody or antigen binding fragment thereof provided herein binds to one or more amino acid residues selected from His 46, Glu 48, Thr 49, Leu 50, Lys 85, Gln 93, Pro 94, Glu 95, Pro 96, Val 97 and Lys 98 of the amino acid sequence of IL-36α represented by SEQ ID NO: 5 or SEQ ID NO:7 and/or one or more amino acid residues selected from Ala 49, Leu 50, Gly 92, Gln 93, Pro 94, Glu 95, Pro 96, Val 97 and Lys 98 of the amino acid sequence of IL-36γ represented by SEQ ID NO: 10.

In some embodiments, the antibody or antigen binding fragment thereof binds to at least one of amino acid residues selected from Leu 50, Gln 93, Pro 94, Glu 95, Pro 96, Val 97 and Lys 98 of both the amino acid sequence of IL-36α represented by SEQ ID NO: 5 or SEQ ID NO: 7 and the amino acid sequence of IL-36γ represented by SEQ ID NO: 10.

In some embodiments, the antibody or antigen binding fragment thereof binds to 2, 3, 4, 5, 6, or 7 amino acid residues selected from Leu 50, Gln 93, Pro 94, Glu 95, Pro 96, Val 97 and Lys 98 of both the amino acid sequence of IL-36α represented by SEQ ID NO: 5 or SEQ ID NO: 7 and the amino acid sequence of IL-36γ represented by SEQ ID NO: 10.

In some embodiments, the antibody or antigen binding fragment thereof binds to the 93rd to 98th amino acid residues of the amino acid sequence of IL-36α represented by SEQ ID NO: 5 or SEQ ID NO: 7 and the amino acid sequence of IL-36γ represented by SEQ ID NO: 10. In other embodiments, the antibody or antigen binding fragment thereof binds to the 50th and 93rd to 98th amino acid residues of the amino acid sequence of IL-36α represented by SEQ ID NO: 5 or SEQ ID NO: 7 and the amino acid sequence of IL-36γ represented by SEQ ID NO: 10.

In some embodiments, the antibody or antigen binding fragment thereof further binds to at least one of amino acid residue selected from Arg 45, His 46, Glu 48, Thr 49, Lys 85, Asp 89, Asn 92 and Phe 100 of the amino acid sequence of IL-36α represented by SEQ ID NO: 5 or SEQ ID NO:7, and/or at least one of amino acid residue selected from Tyr 46, Glu 48, Ala 49, Gln 85, Gly 92 and Phe 100 of the amino acid sequence of IL-36γ represented by SEQ ID NO: 10. In other embodiments, the antibody or antigen binding fragment thereof further binds to at least one of amino acid residues selected from His 46, Glu 48, Thr 49 and Lys 85 of the amino acid sequence of IL-36α represented by SEQ ID NO: 5 or SEQ ID NO: 7, and at least one of IL-36γ amino acid residues selected from Ala 49 and Gly 92 of the amino acid sequence of IL-36γ represented by SEQ ID NO: 10.

In some embodiments, the antibody or antigen binding fragment does not bind to IL-36β. In some embodiments, the antibody or antigen binding fragment does not antagonize IL-36β.

In some embodiments, the antibody or antigen binding fragment does not bind to IL-36Ra. In some embodiments, the antibody or antigen binding fragment does not antagonize IL-36Ra.

In some embodiments, when used in combination with IL-36Ra, the combination of IL-36Ra and the antibody or antigen binding fragment thereof provided herein antagonizes IL-36α, IL-36β and IL-36γ.

In some embodiments, the IL-36α and IL-36γ are human IL-36α and IL-36γ. In other embodiments, the IL-36α and IL-36γ are cynomolgus macaque IL-36α and IL-36γ.

In some embodiments, the antibody or antigen binding fragment thereof binds to human and cynomolgus macaque IL-36α and IL-36γ, and wherein the antibody is an antagonist of human and cynomolgus macaque IL-36α and IL-36γ.

In some embodiments, the antibody or antigen binding fragment thereof does not bind to human or cynomolgus macaque IL-36β. In some embodiments, the antibody or antigen binding fragment thereof does not bind to human or cynomolgus macaque IL-36Ra.

In some embodiments, the antibody or antigen binding fragment binds to human IL-36α with a K_{D} of less than 100 nM as determined by a surface plasmon resonance method, and wherein the antibody or antigen binding fragment thereof binds to human IL-36γ with a K_{D} of less than 100 nM as determined by a surface plasmon resonance method. In some embodiments, the antibody or antigen binding fragment binds to human IL-36α with a K_{D} of less than 10 nM as determined by a surface plasmon resonance method, and wherein the antibody or antigen binding fragment thereof binds to human IL-36γ with a K_{D} of less than 10 nM as determined by a surface plasmon resonance method. In other embodiments, the antibody or antigen binding fragment binds to cynomolgus macaque IL-36α with a K_{D} of less than 100 nM as determined by a surface plasmon resonance method, and wherein the antibody or antigen binding fragment thereof binds to cynomolgus macaque IL-36γ with a K_{D} of less than 100 nM as determined by a surface plasmon resonance method. In yet other embodiments, the antibody or antigen binding fragment binds to cynomolgus macaque IL-36α with a K_{D} of less than 10 nM as determined by a surface plasmon resonance method, and wherein the antibody or antigen binding fragment thereof binds to cynomolgus macaque IL-36γ with a K_{D} of less than 10 nM as determined by a surface plasmon resonance method.

In some embodiments, the antibody or antigen binding fragment thereof attenuates IL-36α mediated signaling and/or IL-36γ mediated signaling. In some embodiments, the antibody or antigen binding fragment thereof attenuates the binding of IL-36α to IL-36 receptor and/or the binding of IL-36γ to IL-36 receptor. In other embodiments, the antibody or antigen binding fragment thereof attenuates IL-36 receptor mediated signaling. In yet other embodiments, the antibody or antigen binding fragment thereof attenuates the production of one or more cytokines and/or chemokines selected from a group consisting of IL-8, IL-6, IL-10, TNFα, IL-1β, CXCL1, CCL5, CCL20, CCL2, CCL3, CCL4, CXCL12, VEGF-A, IL-23, IL-36α, IL-36β, and IL-36γ.

In some embodiments, the antibody or antigen binding fragment thereof antagonizes both IL-36α and IL-36γ activity on an IL-36 receptor expressing cell optionally selected from a group consisting of keratinocytes, dermal fibroblasts, monocytes, and PBMCs.

In some embodiments, the antigen binding fragment is selected from a group consisting of a Fab, a Fab′, a F(ab′)₂, a Fv, a scFv, a dsFv, a diabody, a triabody, a tetrabody, and a multispecific antibody formed from antibody fragments.

In some embodiments, the antibody is a mouse antibody. In other embodiments, the antibody is a fully human antibody. In yet other embodiments, the antibody or antigen binding fragment is a humanized antibody or antigen binding fragment thereof.

In some embodiments, the antibody or antigen binding fragment thereof is recombinantly produced. In some embodiments, the antibody or antigen binding fragment thereof is produced by a hybridoma.

In some embodiments, the antibody or antigen binding fragment thereof provided herein comprises: (a) a heavy chain variable region (VH) comprising (i) VH complementarity determining region 1 (CDR H1) comprising an amino acid sequence selected from a group consisting of SEQ ID NO: 68, SEQ ID NO: 71, SEQ ID NO: 75, and SEQ ID NO: 80; (ii) VH complementarity determining region 2 (CDR H2) comprising an amino acid sequence selected from a group consisting of SEQ ID NO: 69, SEQ ID NO: 73, SEQ ID NO: 76, SEQ ID NO: 78, and SEQ ID NO: 81; and (iii) VH complementarity determining region 3 (CDR H3) comprising an amino acid sequence selected from a group consisting of SEQ ID NO: 70, SEQ ID NO: 72, SEQ ID NO: 74, SEQ ID NO: 77, SEQ ID NO: 79, and SEQ ID NO: 82, and (b) a light chain variable region (VL) comprising (i) VL complementarity determining region 1 (CDR L1) comprising an amino acid sequence selected from a group consisting of SEQ ID NO: 83 and SEQ ID NO: 86; (ii) VL complementarity determining region 2 (CDR L2) comprising an amino acid sequence selected from a group consisting of SEQ ID NO: 84, SEQ ID NO: 87, and SEQ ID NO: 90; and (iii) VL complementarity determining region 3 (CDR L3) comprising an amino acid sequence selected from a group consisting of SEQ ID NO: 85, SEQ ID NO: 88, SEQ ID NO: 89, SEQ ID NO: 91, and SEQ ID NO: 92.

In some embodiments, the antibody or antigen binding fragment thereof provided herein comprises a CDR H1 of SEQ ID NO: 68, a CDR H2 of SEQ ID NO: 69, a CDR H3 of SEQ ID NO: 70, a CDR L1 of SEQ ID NO: 83, a CDR L2 of SEQ ID NO: 84, and a CDR L3 of SEQ ID NO: 85.

In some embodiments, the antibody or antigen binding fragment thereof comprises a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88.

In some embodiments, the antibody or antigen binding fragment thereof comprises a CDR H1 of SEQ ID NO: 71, a CDR H2 of SEQ ID NO: 73, a CDR H3 of SEQ ID NO: 74, a CDR L1 of SEQ ID NO: 86, a CDR L2 of SEQ ID NO: 87, and a CDR L3 of SEQ ID NO: 88.

In some embodiments, the antibody or antigen binding fragment thereof comprises a CDR H1 of SEQ ID NO: 75, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88.

In some embodiments, the antibody or antigen binding fragment thereof comprises a CDR H1 of SEQ ID NO: 75, a CDR H2 of SEQ ID NO: 76, a CDR H3 of SEQ ID NO: 77, a CDR L1 of SEQ ID NO: 83, a CDR L2 of SEQ ID NO: 87, and a CDR L3 of SEQ ID NO: 89.

In other embodiments, the antibody or antigen binding fragment thereof comprises a CDR H1 of SEQ ID NO: 71, a CDR H2 of SEQ ID NO: 78, a CDR H3 of SEQ ID NO: 79, a CDR L1 of SEQ ID NO: 86, a CDR L2 of SEQ ID NO: 90, and a CDR L3 of SEQ ID NO: 91.

In yet other embodiments, the antibody or antigen binding fragment thereof comprises a CDR H1 of SEQ ID NO: 80, a CDR H2 of SEQ ID NO: 81, a CDR H3 of SEQ ID NO: 82, a CDR L1 of SEQ ID NO: 86, a CDR L2 of SEQ ID NO: 87, and a CDR L3 of SEQ ID NO: 92.

In yet other embodiments, the antibody or antigen binding fragment thereof comprises a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 84, and a CDR L3 of SEQ ID NO: 88.

In some embodiments, the antibody or antigen binding fragment thereof comprises a CDR H1 comprising an amino acid sequence of the CDR H1 contained in SEQ ID NO: 23, a CDR H2 comprising an amino acid sequence of the CDR H2 contained in SEQ ID NO: 23; a CDR H3 comprising an amino acid sequence of the CDR H3 contained in SEQ ID NO: 23; a CDR L1 comprising an amino acid sequence of the CDR L1 contained in SEQ ID NO: 51, a CDR L2 comprising an amino acid sequence of the CDR L2 contained in SEQ ID NO: 51; and a CDR L3 comprising an amino acid sequence of the CDR L3 contained in SEQ ID NO: 51.

In other embodiments, the antibody or antigen binding fragment thereof comprises a CDR H1 comprising an amino acid sequence of the CDR H1 contained in SEQ ID NO: 27, a CDR H2 comprising an amino acid sequence of the CDR H2 contained in SEQ ID NO: 27; a CDR H3 comprising an amino acid sequence of the CDR H3 contained in SEQ ID NO: 27; a CDR L1 comprising an amino acid sequence of the CDR L1 contained in SEQ ID NO: 55, a CDR L2 comprising an amino acid sequence of the CDR L2 contained in SEQ ID NO: 55; and a CDR L3 comprising an amino acid sequence of the CDR L3 contained in SEQ ID NO: 55.

In yet other embodiments, the antibody or antigen binding fragment thereof comprises a CDR H1 comprising an amino acid sequence of the CDR H1 contained in SEQ ID NO: 31, a CDR H2 comprising an amino acid sequence of the CDR H2 contained in SEQ ID NO: 31; a CDR H3 comprising an amino acid sequence of the CDR H3 contained in SEQ ID NO: 31; a CDR L1 comprising an amino acid sequence of the CDR L1 contained in SEQ ID NO: 55, a CDR L2 comprising an amino acid sequence of the CDR L2 contained in SEQ ID NO: 55; and a CDR L3 comprising an amino acid sequence of the CDR L3 contained in SEQ ID NO: 55.

In yet other embodiments, the antibody or antigen binding fragment thereof comprises a CDR H1 comprising an amino acid sequence of the CDR H1 contained in SEQ ID NO: 35, a CDR H2 comprising an amino acid sequence of the CDR H2 contained in SEQ ID NO: 35; a CDR H3 comprising an amino acid sequence of the CDR H3 contained in SEQ ID NO: 35; a CDR L1 comprising an amino acid sequence of the CDR L1 contained in SEQ ID NO: 55, a CDR L2 comprising an amino acid sequence of the CDR L2 contained in SEQ ID NO: 55; and a CDR L3 comprising an amino acid sequence of the CDR L3 contained in SEQ ID NO: 55.

In yet other embodiments, the antibody or antigen binding fragment thereof comprises a CDR H1 comprising an amino acid sequence of the CDR H1 contained in SEQ ID NO: 39, a CDR H2 comprising an amino acid sequence of the CDR H2 contained in SEQ ID NO: 39; a CDR H3 comprising an amino acid sequence of the CDR H3 contained in SEQ ID NO: 39; a CDR L1 comprising an amino acid sequence of the CDR L1 contained in SEQ ID NO: 59, a CDR L2 comprising an amino acid sequence of the CDR L2 contained in SEQ ID NO: 59; and a CDR L3 comprising an amino acid sequence of the CDR L3 contained in SEQ ID NO: 59.

In yet other embodiments, the antibody or antigen binding fragment thereof comprises a CDR H1 comprising an amino acid sequence of the CDR H1 contained in SEQ ID NO: 43, a CDR H2 comprising an amino acid sequence of the CDR H2 contained in SEQ ID NO: 43; a CDR H3 comprising an amino acid sequence of the CDR H3 contained in SEQ ID NO: 43; a CDR L1 comprising an amino acid sequence of the CDR L1 contained in SEQ ID NO: 63, a CDR L2 comprising an amino acid sequence of the CDR L2 contained in SEQ ID NO: 63; and a CDR L3 comprising an amino acid sequence of the CDR L3 contained in SEQ ID NO: 63.

In yet other embodiments, the antibody or antigen binding fragment thereof comprises a CDR H1 comprising an amino acid sequence of the CDR H1 contained in SEQ ID NO: 47, a CDR H2 comprising an amino acid sequence of the CDR H2 contained in SEQ ID NO: 47; a CDR H3 comprising an amino acid sequence of the CDR H3 contained in SEQ ID NO: 47; a CDR L1 comprising an amino acid sequence of the CDR L1 contained in SEQ ID NO: 67, a CDR L2 comprising an amino acid sequence of the CDR L2 contained in SEQ ID NO: 67; and a CDR L3 comprising an amino acid sequence of the CDR L3 contained in SEQ ID NO: 67.

In some embodiments, the CDR H1, CDR H2, CDR H3, CDR L1, CDR L2, and CDR L3 are determined according to Kabat numbering. In other embodiments, the CDR H1, CDR H2, CDR H3, CDR L1, CDR L2, and CDR L3 are determined according to AbM numbering. In yet other embodiments, the CDR H1, CDR H2, CDR H3, CDR L1, CDR L2, and CDR L3 are determined according to Chothia numbering. In yet other embodiments, the CDR H1, CDR H2, CDR H3, CDR L1, CDR L2, and CDR L3 are determined according to Contact numbering. In yet other embodiments, the CDR H1, CDR H2, CDR H3, CDR L1, CDR L2, and CDR L3 are determined according to IMGT numbering.

In some embodiments, the antibody or antigen binding fragment thereof comprises a VH region comprising an amino acid sequence of SEQ ID NO: 23, and a VL region comprising an amino acid sequence of SEQ ID NO: 51. In other embodiments, the antibody or antigen binding fragment thereof comprises a VH region comprising an amino acid sequence of SEQ ID NO: 27, and a VL region comprising an amino acid sequence of SEQ ID NO: 55. In yet other embodiments, the antibody or antigen binding fragment thereof comprises a VH region comprising an amino acid sequence of SEQ ID NO: 31, and a VL region comprising an amino acid sequence of SEQ ID NO: 55. In yet other embodiments, the antibody or antigen binding fragment thereof comprises a VH region comprising an amino acid sequence of SEQ ID NO: 35, and a VL region comprising an amino acid sequence of SEQ ID NO: 55. In yet other embodiments, the antibody or antigen binding fragment thereof comprises a VH region comprising an amino acid sequence of SEQ ID NO: 39, and a VL region comprising an amino acid sequence of SEQ ID NO: 59. In yet other embodiments, the antibody or antigen binding fragment thereof comprises a VH region comprising an amino acid sequence of SEQ ID NO: 43, and a VL region comprising an amino acid sequence of SEQ ID NO: 63. In yet other embodiments, the antibody or antigen binding fragment thereof comprises a VH region comprising an amino acid sequence of SEQ ID NO: 47, and a VL region comprising an amino acid sequence of SEQ ID NO: 67.

In some embodiments, the antibody or antigen binding fragment thereof provided herein comprises (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1, Lys 12, Val 20, Tyr 27, Thr 28, Phe 29, Thr 30, Arg 38, Met 48, Arg 67, Val 68, Ala 72, Ser 77, Ala 79, Met 81, Leu 83 and Val 117; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77 and Asp 87.

In some embodiments, the antibody or antigen binding fragment thereof comprises (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ala 72 with Thr, Ser 77 with Asp, Ala 79 with Val, Met 81 with Leu, Leu 83 with Phe and Val 117 with Leu; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Ala, Thr 48 with Gly, Ser 51 with Gly, Trp 59 with Gly, Thr 60 with Val, Leu 77 with Ile, and Asp 87 with Ile.

In some embodiments, the antibody or antigen binding fragment thereof comprises (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln 1, Lys 12, Val 20, Tyr 27, Thr 28, Phe 29, Thr 30, Arg 38, Met 48, Arg 67, Val 68, Ile 70, Ala 72, Ser 77, Met 81, and Val 117; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77, and Asp 87.

In some embodiments, the antibody or antigen binding fragment thereof comprises (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ile 70 with Leu, Ala 72 with Thr, Ser 77 with Asn, Met 81 with Leu, and Val 117 with Leu; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Thr, Thr 48 with Gly, Ser 51 with Gly, Trp 59 with Gly, Thr 60 with Val, Leu 77 with Ile, and Asp 87 with Ile.

In some embodiments, the antibody or antigen binding fragment thereof comprises (i) a VH region comprising an amino acid sequence selected from SEQ ID NOs: 115, 139, 140, 141, 142, 143, 144, 145, 146, 147, 148, 149, 150, 151, 152, 153, 154, 155, 156, 157, 158, 159, 160, 161, 162 and 163, and (ii) a VL region comprising an amino acid sequence selected from SEQ ID NOs: 114, 116, 117, 118, 119, 120, 121, 122, 123, 124, 125, 126, 127, 128, 129, 130, 131, 132, 133, 134, 135, 136, 137 and 138.

In some embodiments, the antibody or antigen binding fragment thereof comprises (i) a VH region comprising an amino acid sequence selected from SEQ ID NOs: 165, 171, 172, 173, 174, 175, 176 and 177, and (ii) a VL region comprising an amino acid sequence selected from SEQ ID NOs: 164, 166, 167, 168, 169 and 170.

In some embodiments, the antibody or antigen binding fragment thereof comprises a VH region comprising an amino acid sequence of SEQ ID NO: 161, and a VL region comprising an amino acid sequence of SEQ ID NO: 130. In other embodiments, the antibody or antigen binding fragment thereof comprises a VH region comprising an amino acid sequence of SEQ ID NO: 161, and a VL region comprising an amino acid sequence of SEQ ID NO: 136. In yet other embodiments, the antibody or antigen binding fragment thereof comprises a VH region comprising an amino acid sequence of SEQ ID NO: 161, and a VL region comprising an amino acid sequence of SEQ ID NO: 137. In yet other embodiments, the antibody or antigen binding fragment thereof comprises a VH region comprising an amino acid sequence of SEQ ID NO: 161, and a VL region comprising an amino acid sequence of SEQ ID NO: 138. In yet other embodiments, the antibody or antigen binding fragment thereof comprises a VH region comprising an amino acid sequence of SEQ ID NO: 176 and a VL region comprising an amino acid sequence of SEQ ID NO: 166. In yet other embodiments, the antibody or antigen binding fragment thereof comprises a VH region comprising an amino acid sequence of SEQ ID NO: 176, and a VL region comprising an amino acid sequence of SEQ ID NO: 167. In yet other embodiments, the antibody or antigen binding fragment thereof comprises a VH region comprising an amino acid sequence of SEQ ID NO: 174, and a VL region comprising an amino acid sequence of SEQ ID NO: 167. In yet other embodiments, the antibody or antigen binding fragment thereof comprises a VH region comprising an amino acid sequence of SEQ ID NO: 175, and a VL region comprising an amino acid sequence of SEQ ID NO: 167. In some embodiments, the above mentioned antibody is a humanized antibody.

In some embodiments, the antibody or antigen binding fragment thereof is conjugated to an agent. In some embodiments, the agent is selected from a group consisting of a cytotoxic agent, a radioisotope, a metal chelator, an enzyme, a fluorescent compound, a bioluminescent compound, and a chemiluminescent compound.

In another aspect, provided herein is a pharmaceutical composition comprising the antibody or antigen binding fragment provided herein and a pharmaceutically acceptable excipient.

In yet another aspect, provided herein is a method of treating and/or preventing a disease or disorder comprising administering a therapeutically effective amount of the antibody or antigen binding fragment thereof provided herein to a subject.

In some embodiments, the disease or disorder is a disease or disorder mediated by IL-36α and/or IL-36γ. In some embodiments, the disease or disorder is an inflammatory disease or an autoimmune disease. In other embodiments, the disease or disorder is related to skin tissue, intestinal tissue and/or lung tissue. In some embodiments, the disease or disorder is selected from a group consisting of generalized pustular psoriasis, palmoplantar pustulosis, palmoplantar pustular psoriasis, discoid lupus erythematosus, lupus erythematosus, atopic dermatitis, Crohn's disease, ulcerative colitis, asthma, inflammatory bowel diseases, psoriasis vulgaris, acrodermatitis continua of Hallopeau, acute generalized exanthematous pustulosis, hidradenitis suppurativa, lichen planus, Sjögren's syndrome, rheumatoid arthritis, psoriatic arthritis, chronic rhinosinusitis, acne vulgaris, impetigo herpetiformis, pyoderma gangrenosum, and polymorphic light eruption. In some embodiments, the subject is a human subject.

In yet another aspect, provided herein is a polynucleotide comprising nucleotide sequences encoding the antibody or antigen binding fragment thereof provided herein or a portion thereof.

In some embodiments, the polynucleotide comprises a nucleotide sequence selected from a group consisting of SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 48, SEQ ID NO: 52, SEQ ID NO: 56, SEQ ID NO: 60, SEQ ID NO: 64, SEQ ID NO: 50, SEQ ID NO: 54, SEQ ID NO: 58, SEQ ID NO: 62, and SEQ ID NO: 66.

In some embodiments, the polynucleotide comprises a nucleotide sequence of SEQ ID NO: 22 and/or a nucleotide sequence of SEQ ID NO: 50. In some embodiments, the polynucleotide comprises a nucleotide sequence of SEQ ID NO: 26 and/or a nucleotide sequence of SEQ ID NO: 54. In other embodiments, the polynucleotide comprises a nucleotide sequence of SEQ ID NO: 30 and/or a nucleotide sequence of SEQ ID NO: 54. In other embodiments, the polynucleotide comprises a nucleotide sequence of SEQ ID NO: 34 and/or a nucleotide sequence of SEQ ID NO: 54. In yet other embodiments, the polynucleotide comprises a nucleotide sequence of SEQ ID NO: 38 and/or a nucleotide sequence of SEQ ID NO: 58. In yet other embodiments, the polynucleotide comprises a nucleotide sequence of SEQ ID NO: 42 and/or a nucleotide sequence of SEQ ID NO: 62. In yet other embodiments, the polynucleotide comprises a nucleotide sequence of SEQ ID NO: 46 and/or a nucleotide sequence of SEQ ID NO: 66. In yet other embodiments, the polynucleotide comprises a nucleotide sequence of SEQ ID NO: 20 and/or a nucleotide sequence of SEQ ID NO: 48. In yet other embodiments, the polynucleotide comprises a nucleotide sequence of SEQ ID NO: 24 and/or a nucleotide sequence of SEQ ID NO: 52. In yet other embodiments, the polynucleotide comprises a nucleotide sequence of SEQ ID NO: 28 and/or a nucleotide sequence of SEQ ID NO: 52. In yet other embodiments, the polynucleotide comprises a nucleotide sequence of SEQ ID NO: 32 and/or a nucleotide sequence of SEQ ID NO: 52. In yet other embodiments, the polynucleotide comprises a nucleotide sequence of SEQ ID NO: 36 and/or a nucleotide sequence of SEQ ID NO: 56. In yet other embodiments, the polynucleotide comprises a nucleotide sequence of SEQ ID NO: 40 and/or a nucleotide sequence of SEQ ID NO: 60. In yet other embodiments, the polynucleotide comprises a nucleotide sequence of SEQ ID NO: 44 and/or a nucleotide sequence of SEQ ID NO: 64.

In another aspect, provided herein is a vector comprising the polynucleotide provided herein.

In yet another aspect, provided herein is a cell comprising the polynucleotide provided herein. In some embodiments, provided herein is a cell comprising the vector provided herein. In some embodiments, provided herein is a cell which is transformed by the vector provided herein.

In yet another aspect, provided herein is a hybridoma which generates the antibody or the antibody fragment thereof provided herein.

In yet another aspect, provided herein is a method of making an antibody or antigen binding fragment thereof comprising culturing the cell or the hybridoma provided herein to express the antibody or antigen binding fragment thereof.

### 4. BRIEF DESCRIPTION OF THE FIGURES

FIG. 1A to 1F depict the results of HaCaT functional assays demonstrating that IL-36α and IL-36γ dual-antagonist antibodies antagonize human IL-36α and IL-36γ. FIG. 1A depicts the results of the HaCaT assay described in Example 1, in which cells were stimulated with 10 nM of human IL-36α in the presence of a titration of IL-36Ra or IL-36α and IL-36γ dual-antagonist antibodies. FIG. 1B depicts the results of the HaCaT assay described in Example 1, in which cells were stimulated with 10 nM of human IL-36α in the presence of a titration of IL-36Ra or IL-36α and IL-36γ dual-antagonist antibodies. FIG. 1C depicts the results of the HaCaT assay described in Example 1, in which cells were stimulated with 10 nM of human IL-36α in the presence of a titration of IL-36Ra or IL-36α and IL-36γ dual-antagonist antibodies. FIG. 1D depicts the results of the HaCaT assay described in Example 1, in which cells were stimulated with 10 nM of human IL-36γ in the presence of a titration of IL-36Ra or IL-36α and IL-36γ dual-antagonist antibodies. FIG. 1E depicts the results of the HaCaT assay described in Example 1, in which cells were stimulated with 10 nM of human IL-36γ in the presence of a titration of IL-36Ra or IL-36α and IL-36γ dual-antagonist antibodies. FIG. 1F depicts the results of the HaCaT assay described in Example 1, in which cells were stimulated with 10 nM of human IL-36γ in the presence of a titration of IL-36Ra or IL-36α and IL-36γ dual-antagonist antibodies.

FIG. 2A to 2D depict the results of HaCaT functional assays demonstrating that IL-36α and IL-36γ dual-antagonist antibodies can antagonize cynomolgus macaque IL-36α and cynomolgus macaque IL-36γ. FIG. 2A depicts the results of the HaCaT assay described in Example 1, in which cells were stimulated with 10 nM of cynomolgus macaque IL-36α in the presence of a titration of IL-36Ra or IL-36α and IL-36γ dual-antagonist antibodies. FIG. 2B depicts the results of the HaCaT assay described in Example 1, in which cells were stimulated with 10 nM of cynomolgus macaque IL-36α in the presence of a titration of IL-36Ra or IL-36α and IL-36γ dual-antagonist antibodies. FIG. 2C depicts the results of the HaCaT assay described in Example 1, in which cells were stimulated with 10 nM of cynomolgus macaque IL-36γ in the presence of a titration of IL-36Ra or IL-36α and IL-36γ dual-antagonist antibodies. FIG. 2D depicts the results of the HaCaT assay described in Example 1, in which cells were stimulated with 10 nM of cynomolgus macaque IL-36γ in the presence of a titration of IL-36Ra or IL-36α and IL-36γ dual-antagonist antibodies.

FIG. 3 depicts the results of the HaCaT functional assay described in Example 1 demonstrating that IL-36α and IL-36γ dual-antagonist antibodies do not antagonize human IL-36β, in which cells were stimulated with 10 nM of human IL-36β in the presence of a titration of human IL-36Ra or IL-36α and IL-36γ dual-antagonist antibodies.

FIG. 4A to 4D depict the results of a primary human keratinocyte functional assay demonstrating that IL-36α and IL-36γ dual-antagonist antibodies can antagonize IL-36α and IL-36γ. FIG. 4A depicts the results of the primary human keratinocyte assay described in EXAMPLE 3, in which cells were stimulated with 6.25 nM of human IL-36α in the presence of a titration of IL-36Ra or IL-36α and IL-36γ dual-antagonist antibodies. FIG. 4B depicts the results of the primary human keratinocyte assay described in EXAMPLE 3, in which cells were stimulated with 8 nM of human IL-36α in the presence of a titration of IL-36Ra or IL-36α and IL-36γ dual-antagonist antibodies. FIG. 4C depicts the results of the primary human keratinocyte assay described in EXAMPLE 3, in which cells were stimulated with 8.4 nM of human IL-36γ in the presence of a titration of IL-36Ra or IL-36α and IL-36γ dual-antagonist antibodies. FIG. 4D depicts the results of the primary human keratinocyte assay described in EXAMPLE 3, in which cells were stimulated with 8.4 nM of human IL-36γ in the presence of a titration of IL-36Ra or IL-36α and IL-36γ dual-antagonist antibodies.

FIG. 5A to 5D depicts the results of a primary human monocyte functional assay demonstrating that IL-36α and IL-36γ dual-antagonist antibodies can antagonize IL-36α and IL-36γ. FIG. 5A depicts the results of the primary human monocyte assay described in EXAMPLE 3, in which cells were stimulated with 20 nM of human IL-36α in the presence of a titration of IL-36Ra or IL-36α and IL-36γ dual-antagonist antibodies. FIG. 5B depicts the results of the primary human monocyte assay described in EXAMPLE 3, in which cells were stimulated with 40 nM of human IL-36α in the presence of a titration of IL-36Ra or IL-36α and IL-36γ dual-antagonist antibodies. FIG. 5C depicts the results of the primary human monocyte assay described in EXAMPLE 3, in which cells were stimulated with 6 nM of human IL-36γ in the presence of a titration of IL-36Ra or IL-36α and IL-36γ dual-antagonist antibodies. FIG. 5D depicts the results of the primary human monocyte assay described in EXAMPLE 3, in which cells were stimulated with 50 nM of human IL-36γ in the presence of a titration of IL-36Ra or IL-36α and IL-36γ dual-antagonist antibodies.

FIG. 6A to 6B depict the results of a primary cynomolgus macaque keratinocyte functional assay demonstrating that IL-36α and IL-36γ dual-antagonist antibodies can antagonize IL-36α and IL-36γ. FIG. 6A depicts the results of the primary cynomolgus macaque keratinocyte assay described in EXAMPLE 3, in which cells were stimulated with a titration of cynomolgus macaque IL-36α in the presence of 200 nM IL-36Ra or 200 nM IL-36α and IL-36γ dual-antagonist antibodies. FIG. 6B depicts the results of the primary cynomolgus macaque keratinocyte assay described in EXAMPLE 3, in which cells were stimulated with a titration of cynomolgus macaque IL-36γ in the presence of 200 nM IL-36Ra or 200 nM IL-36α and IL-36γ dual-antagonist antibodies.

FIG. 7 depicts the results of the HaCaT assay described in EXAMPLE 4 demonstrating that IL-36α and IL-36γ dual-antagonist antibodies can simultaneously antagonize IL-36α and IL-36γ. Cells were stimulated with a titration of human IL-36α and IL-36γ in the presence of various amounts of IL-36Ra or chimeric m/h 144D464A. Secreted IL-8 was measured by ELISA and the O.D. values are depicted in a greyscale heat map with higher O.D. values (i.e., higher IL-8 levels) corresponding to a darker color.

FIG. 8A to 8B depict the results of HaCaT assays demonstrating that IL-36α and IL-36γ dual-antagonist antibodies alone, or in complex with IL-36α or IL-36γ, do not impact IL-36β agonist activity. FIG. 8A depicts the results of the experimental controls utilized in the HaCaT assay described in EXAMPLE 4. FIG. 8B depicts the results of the HaCaT assay described in EXAMPLE 4 in which cells were stimulated with a titration of human IL-36β in the presence of IL-36Ra, chimeric m/h 144D464A, chimeric m/h 144D464A pre-incubated with IL-36α, or chimeric m/h 144D464A pre-incubated with IL-36γ.

FIG. 9A to 9D depict the results of HaCaT assays demonstrating that IL-36α and IL-36γ dual-antagonist antibodies do not interfere with IL-36Ra antagonist activity and can cooperate with IL-36Ra to suppress IL-36α, IL-36β, and IL-36γ. FIG. 9A depicts the results of the HaCaT assay described in EXAMPLE 4 in which cells were stimulated with a titration of human IL-36α in the presence of 100 nM IL-36Ra, 100 nM chimeric m/h 144D464A, or a mixture of 100 nM IL-36Ra and 100 nM chimeric m/h 144D464A. FIG. 9B depicts the results of the HaCaT assay described in EXAMPLE 4 in which cells were stimulated with a titration of human IL-36α in the presence of 100 nM IL-36Ra, 100 nM chimeric m/h 144D464A, or a mixture of 100 nM IL-36Ra and 100 nM chimeric m/h 144D464A. FIG. 9C depicts the results of the HaCaT assay described in EXAMPLE 4 in which cells were stimulated with a titration of human IL-36γ in the presence of 100 nM IL-36Ra, 100 nM chimeric m/h 144D464A, or a mixture of 100 nM IL-36Ra and 100 nM chimeric m/h 144D464A. FIG. 9D depicts the results of the HaCaT assay described in EXAMPLE 4 in which cells were stimulated with a combined titration of human IL-36α, human IL-36β, and human IL-36γ in the presence of 100 nM IL-36Ra, 100 nM chimeric m/h 144D464A, or a mixture of 100 nM IL-36Ra and 100 nM chimeric m/h 144D464A.

FIG. 10 depicts the amino acid sequences of variable regions of light chains of a mouse antibody 144D464A and humanized 144D464A antibodies, which do not include signal sequences. The regions surrounded by frames in each sequence show CDR sequences.

FIG. 11 depicts the amino acid sequences of variable regions of heavy chains of a mouse antibody 144D464A and humanized 144D464A antibodies, which do not include signal sequences. The regions surrounded by frames in each sequence show CDR sequences.

FIG. 12 depicts the amino acid sequences of variable regions of light chains of a mouse antibody 144L249B and humanized 144L249B antibodies, which do not include signal sequences. The regions surrounded by frames in each sequence show CDR sequences.

FIG. 13 depicts the amino acid sequences of variable regions of heavy chains of a mouse antibody 144L249B and humanized 144L249B antibodies, which do not include signal sequences. The regions surrounded by frames in each sequence show CDR sequences.

FIG. 14A and 14B depict the results of HaCaT functional assays demonstrating that humanized 144D464A antibodies antagonize human IL-36α and IL-36γ. FIG. 14A depicts the results of the HaCaT assay, in which cells were stimulated with 10 nM of human IL-36α or IL-36γ in the presence of a titration of humanized 144D464A antibody LV7a HV10b. FIG. 14B depicts the results of the HaCaT assay, in which cells were stimulated with 10 nM of human IL-36α or IL-36γ in the presence of a titration of a mouse antibody 144D464A or humanized 144D464A antibody LV9are HV10b.

FIG. 15 depicts the results of HaCaT functional assays demonstrating that humanized 144L249B antibodies antagonize human IL-36α and IL-36γ, in which cells were stimulated with 10 nM of human IL-36α or IL-36γ in the presence of a titration of a mouse antibody 144L249B, or humanized 144L249B antibodies LV7a HV11, LV9 HV11, LV9 HV10b or LV9 HV10c.

FIG. 16 depicts crystal structure of IL-36α-L249B Fab complex. Part A shows cartoon representation of IL-36α-L249B Fab complex (V_{H}, variable region of heavy chain. C_{H}, constant region of heavy chain. V_{L}, variable region of light chain. C_{L}, constant region of light chain). Part B shows specific interacting residues in the interface between CDR loops of L249B Fab (left) and the loops of IL-36α (right). All interacting residues from CDR loops of L249B Fab and IL-36α are represented as sticks. In each panel, the respective CDR loops of HC and LC are labeled. The hydrogen bonds and hydrophobic contacts are shown as dashed lines.

FIG. 17 depicts crystal structure of IL-36γ-L249B Fab complex and comparison to IL-36α-L249B Fab complex. Part A shows superposition of IL-36γ-L249B Fab complex with IL-36α-L249B Fab complex. The L249B Fab, IL-36γ and IL-36α are represented as cartoons (V_{H}, variable region of heavy chain. C_{H}, constant region of heavy chain. V_{L}, variable region of light chain. C_{L}, constant region of light chain). Part B shows comparison of interaction interface between IL-36γ and IL-36α with L249B Fab, representing the binding of cytokine at the crevice formed by variable loops of HC and LC (transparent surface) of L249B Fab. Part C shows interactions between CDR loops of L249B Fab (left) and IL-36γ (right). In all panels, black dashed lines indicate hydrogen bonds and hydrophobic contacts. The respective CDR loops of L249B Fab are labeled in each panel. The far right panels are superposition of the interactions between CDR loops of IL-36γ-L249B Fab complex with IL-36α-L249B Fab complex, with the residues of IL-36α from the IL-36α-L249B Fab complex labeled with *.

FIG. 18 depicts binding footprint of L249B Fab on the surface of IL-36α (part A) and IL-36γ (part B). The IL-36α and IL-36γ residues that interact with HC or LC CDR loops are labeled in black in both panels. The residues of IL-36α and IL-36γ that interact with both L249B HC and LC are labeled in white.

FIG. 19 depicts crystal structure of IL-36α-D464A Fab complex. Part A shows cartoon representation of IL-36α-D464A Fab complex (V_{H}, variable region of heavy chain. C_{H}, constant region of heavy chain. V_{L}, variable region of light chain. C_{L}, constant region of light chain). Part B shows interaction interface between IL-36α (cartoon) and D464A Fab (transparent surface) representing the binding of IL-36α at the crevice formed by variable loops of heavy chain and light chain region of D464A Fab (VH, variable region of heavy chain. VL, variable region of light chain). Part C shows specific interacting residues in the interface between heavy chain CDR loop H1 (left panel) and H2 (right panel) of D464A Fab (left) and the loops of IL-36α (right). Part D shows interaction of D464A heavy chain CDR loop H3 with IL-36α (right panel) and light chain CDR loops (left) with IL-36α (right) (left panel). All interacting residues from CDR loops of D464A Fab and those of IL-36α are represented as sticks. In each panel, the respective CDR loops of heavy chain and light chain are labeled. The hydrogen bonds are shown as dashed lines.

FIG. 20 depicts crystal structure of IL-36γ-D464A Fab complex and comparison to IL-36α-D464A Fab complex. Part A shows superposition of IL-36γ-D464A Fab complex with IL-36α-D464A Fab complex. The variable heavy chain and light chain of D464A Fab in both complexes are shown as transparent surface; IL-36γ and IL-36α as cartoons (V_{H}, variable region of heavy chain. V_{L}, variable region of light chain). Part B shows structural superposition of IL-36α and IL-36γ, both in complex with D464A Fab, showing similar overall topological architecture. The N-terminal, C-terminal ends and twelve β-strands are marked. Polar interactions from three residues His 46, Asp 89 and Lys 85 that are present in IL-36α-D464A Fab complex but missing in IL-36γ-D464A Fab complex are labeled. Part C shows D464A Fab foot print on the surface of IL-36γ (top) and IL-36α (bottom). The IL-36γ and IL-36α residues that interact with D464A heavy chain CDR loops and light chain CDR loops are labeled. Part D left panel shows the interactions between H3 loop of D464A Fab (left side) and IL-36γ (right side) and the structure superposition with D464A Fab complexed with IL-36α. Lys 85 of IL-36α and Asn 104 of D464A Fab from the IL-36α-D464A Fab complex are labeled with *. Part D right panel shows the interactions between H2 loop of D464A Fab (left side) and IL-36γ (right side) and the structure superposition with D464A Fab complexed with IL-36a. His 46 and Glu 48 of IL-36α and Arg 59 of D464A Fab from the IL-36α-D464A Fab complex are labeled with *. In both panels, black dashed lines indicate hydrogen bonds.

### 5. DETAILED DESCRIPTION

The present disclosure provides novel Interleukin 36 (IL-36) antibodies, pharmaceutical compositions comprising same, and uses thereof. More specifically, the present disclosure provides antibodies antagonizing IL-36α and/or IL-36γ, pharmaceutical compositions comprising these antibodies, and uses thereof.

IL-36 cytokine family is comprised of IL-36 Receptor antagonist (IL-36Ra), IL-36α, IL-36β, and IL-36γ (formerly known as IL-1F5, IL-1F6, IL-1F8, and IL-1F9, respectively) (see Dinarello, C., et al., Nat Immunol, 2010, 11(11): 973). These cytokines are ligands for the IL-36 receptor, which is a heterodimer comprised of IL-36R (also known as IL-1Rrp2) and IL-1RAcP (also known as IL-1 receptor accessory protein). IL-36α, IL-36β, and IL-36γ are agonists to this receptor while IL-36Ra is an antagonist (see Towne, J. E., et al., J Biol Chem, 2004, 279(14): 13677-88; and Blumberg, H., et al., J Exp Med, 2007, 204(11): 2603-14).

For IL-36Ra, IL-36α, IL-36β, and IL-36γ to become fully active they require proteolytic processing and removal of a small stretch of N-terminal amino acids (see Towne, J. E., et al., J Biol Chem, 2011, 286(49): 42594-602). A number of proteases have been identified as being capable of processing the IL-36 cytokines into their truncated, fully active form, including elastase, cathepsin G, cathepsin S, and proteinase-3 (see Clancy, D. M., et al., FEBS J, 2017, 284(11): 1712-1725; Henry, C. M., et al., Cell Rep, 2016. 14(4): 708-722; Ainscough, J. S., et al., Proc Natl Acad Sci USA, 2017. 114(13): E2748-E2757; Macleod, T., et al., Sci Rep, 2016, 6: 24880).

Binding of IL-36α, IL-36β, or IL-36γ to its receptor induces intracellular signaling leading to activation of mitogen-activated protein kinase (MAPK) pathways and nuclear factor kappa B (NF-κB) dependent transcription, resulting in pro-inflammatory gene expression and cytokine production (Towne, J. E., et al., J Biol Chem, 2004, 279(14): 13677-88; and Gabay, C. and J. E. Towne, J Leukoc Biol, 2015, 97(4): 645-52).

The IL-36 receptor and cytokines are expressed in numerous tissues and by various cell types, including the skin, lung, and gut, as well as cells of the immune system, such as monocytes, macrophages, dendritic cells, and T cells (see Gabay, C. and J. E. J Leukoc Biol, 2015, 97(4): p. 645-52; Bassoy, E. Y., et al., Immunol Rev, 2018, 281(1): 169-178; Walsh, P. T. and P. G. Fallon, Ann N Y Acad Sci, 2018, 1417(1): 23-34).

The IL-36 family of cytokines and their receptor have been implicated in numerous inflammatory conditions and diseases. Mutations in IL-36Ra that reduce its stability and functional antagonist activity have been linked to the development of generalized pustular psoriasis (GPP), which is a severe form of psoriasis and can be life threatening (see Marrakchi, S., et al., N Engl J Med, 2011, 365(7): 620-8; Onoufriadis, A., et al., Am J Hum Genet, 2011, 89(3): 432-7; and Tauber, M., et al., J Invest Dermatol, 2016. 136(9):1811-9).

Increased expression of IL-36 (particularly IL-36γ and IL-36α) have been detected in lesional skin from patients with GPP, as well as in other types of psoriasis such as plaque psoriasis, palmoplantar pustular psoriasis, and palmoplantar pustulosis (see Liang, Y., et al., J Allergy Clin Immunol, 2017. 139(4): 1217-1227; Bissonnette, R., et al., PLoS One, 2016. 11(5): e0155215; Johnston, A., et al., J Allergy Clin Immunol, 2017, 140(1): 109-120; D'Erme, A. M., et al., J Invest Dermatol, 2015, 135(4): 1025-1032; and Carrier, Y., et al., J Invest Dermatol, 2011. 131(12): 2428-37). Increased levels of IL-36γ can also be detected in lesional skin from patients with discoid lupus erythematosus and subacute cutaneous lupus erythematosus (see D'Erme, A. M., et al., J Invest Dermatol, 2015. 135(4): 1025-1032; and Jabbari, A., et al., J Invest Dermatol, 2014. 134(1): 87-95). In addition, increased expression of IL-36 cytokines has been detected in acute generalized exanthematous pustulosis as well as in the lesional skin from patients diagnosed with hidradenitis suppurativa (see Liang, Y., et al., J Allergy Clin Immunol, 2017, 139(4): 1217-1227; and Thomi, R., et al., J Eur Acad Dermatol Venereol, 2017, 31(12): 2091-2096; and Hessam, S., et al., Br J Dermatol, 2018, 178(3): 761-767).

Animal models also support the role of IL-36 cytokines in inflammatory skin conditions. Transgenic mice engineered to over-express IL-36α in keratinocytes are born with an inflammatory skin phenotype, which is dependent on a functional IL-36 receptor. This phenotype was exacerbated in mice also lacking IL-36Ra (see Blumberg, H., et al., J Exp Med, 2007, 204(11): 2603-14). Mice over-expressing IL-36α were also more sensitive to skin irritant 12-O-tetradecanoylphorbol 13-acetate (see Blumberg, H., et al., J Immunol, 2010, 185(7): 4354-62). Furthermore, in an imiquimod-based mouse model of psoriasis, mice lacking expression of IL-36α exhibit significantly reduced skin pathology compared to wild-type mice (see Milora, K. A., et al., J Invest Dermatol, 2015, 135(12): 2992-3000).

While there is a substantial evidence to indicate that IL-36 plays an important role in the development of inflammatory skin conditions, the IL-36 pathway has also been observed to be active in other diseases and tissues. For example, elevated expression of IL-36α and IL-36γ has been measured from involved tissues from patients with inflammatory bowel disease including Crohn's disease and ulcerative colitis (see Russell, S. E., et al., Mucosal Immunol, 2016, 9(5): 1193-204; Nishida, A., et al., Inflamm Bowel Dis, 2016. 22(2): 303-14; and Boutet, M. A., et al., Clin Exp Immunol, 2016, 184(2): p. 159-73). Moreover, all three IL-36 agonists (i.e., IL-36α, IL-36β, IL-36γ) were detected in the synovium of patients with rheumatoid arthritis (see Boutet, M. A., et al., Clin Exp Immunol, 2016, 184(2): 159-73).

While IL-36β has been shown to be a strong inducer of anti-microbial peptides and appears to play a protective role against HSV-1 infection (see Johnston, A., et al., J Immunol, 2011. 186(4): 2613-22; and Milora, K. A., et al., Sci Rep, 2017. 7(1): 5799), the roles of IL-36α and IL-36γ are most apparent in inflammatory skin diseases. Thus, there is a need for new therapeutic agents capable of specifically antagonizing IL-36α and/or IL-36γ.

In addition, although antibodies to IL-36 α or IL-36γ are known in the art, such as clone 4 (cat# 10607-MM04, Sino Biological, Wayne, Pa.); clone 1E4 (cat# LS-C139455, LifeSpan BioSciences, Seattle, Wash.); clone 278706 (cat# MAB2320-SP, R&D Systems, Minneapolis, Minn.); clone 2P38 (cat# MBS690041, MyBiosource, San Diego, Calif.); clone 2P38 (cat# GTX52842, GeneTex, Irvine, Calif.); clone MM0388-2P38 (cat# NBP2-11688, Novus Biologicals, Littleton, Colo.); clone 14L515 (cat# 216611, United States Biological, Salem, Mass.); clone 8A11 (cat# ABIN396796, Antibodies Online, Atlanta, Ga.); clone Y-12 (cat# sc-80056, Santa Cruz Biotechnology, Dallas, Tex.); clone 2A8 (cat# LS-C139453, LifeSpan BioSciences, Seattle, Wash.), none of these antibodies is a dual antagonist to IL-36α and IL-36γ. There remains a need for new therapeutic agents capable of specifically antagonizing IL-36α and IL-36γ and which have functional activity.

As demonstrated in Section 6 below, in certain embodiments, the antibodies provided herein are anti-IL-36α and anti-IL-36γ dual-antagonist monoclonal antibodies. The antibodies bind to both human and cynomolgus macaque IL-36α and IL-36γ with high affinity (e.g., with a K_{D} of less than 10 nM for each of IL-36α and IL-36γ). The antibodies antagonize IL-36α and IL-36γ signaling through the IL-36 receptor, which is demonstrated with in vitro functional assays utilizing an immortalized human keratinocyte cells line, primary human keratinocytes, primary human monocytes, human peripheral mononuclear cells, and primary cynomolgus macaque keratinocytes. As shown, certain IL-36α and IL-36γ dual-antagonist antibodies provided herein simultaneously antagonize both IL-36α and IL-36γ without impacting IL-36β signaling or the antagonist activity of IL-36Ra. The above mentioned and other properties make the antibodies provided herein advantageous candidates for treating various diseases or conditions, e.g., inflammatory skin diseases.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dithel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “antagonist” antibody is one which inhibits or reduces biological activity of the antigen it binds. For example, antagonist antibodies may substantially or completely inhibit the biological activity of the antigen. As used herein, an “antagonist” or “inhibitor” of IL-36α or IL-36γ refers to a molecule that is capable of inhibiting or otherwise decreasing one or more of the biological activities of IL-36α or IL-36γ, such as in a cell expressing IL-36α or IL-36γ or in a cell expressing an IL-36α or IL-36γ ligand, such as an IL-36 receptor. For example, in certain embodiments, antibodies provided herein are antagonist antibodies that inhibit the activity of IL-36α and/or IL-36γ on a cell expressing an IL-36 receptor when said antibody is exposed to said cell. In some embodiments, an antagonist of IL-36α or IL-36γ (e.g., an antagonistic antibody provided herein) may, for example, act by inhibiting or otherwise decreasing the activation and/or cell signaling pathways of the cell expressing an IL-36 receptor, thereby inhibiting or limiting an IL-36α or IL-36γ mediated biological activity of the cell relative to the IL-36α or IL-36γ-mediated biological activity in the absence of antagonist. In certain embodiments, the antibodies provided herein are mouse dual antagonistic anti- IL-36α and anti-IL-36γ antibodies. In certain embodiments, the antibodies provided herein are fully human or humanized dual antagonistic anti- IL-36α and anti-IL-36γ antibodies.

An antagonist antibody as used herein is in contrast with an “agonist” antibody, which is an antibody that triggers a response, e.g., one that mimics at least one of the functional activities of a polypeptide of interest (e.g., IL-36α or IL-36γ). An agonist antibody includes an antibody that is a ligand mimetic, for example, wherein a ligand binds to a cell surface receptor and the binding induces cell signaling or activities via an intercellular cell signaling pathway and wherein the antibody induces a similar cell signaling or activation. An “agonist” of IL-36α and IL-36γ refers to a molecule that is capable of activating or otherwise increasing one or more of the biological activities of IL-36α or IL-36γ, such as on a cell that is responsive to IL-36α or IL-36γ through its expression of an IL-36 receptor. In some embodiments, an agonist of IL-36α or IL-36γ may, for example, act by increasing the activity of IL-36α or IL-36γ, leading to an increase in the activation and/or cell signaling pathways of a cell expressing an IL-36 receptor, thereby increasing an IL-36α or IL-36y-mediated biological activity of the cell relative to the IL-36α or IL-36γ-mediated biological activity in the absence of agonist.

An “intact” antibody is one comprising an antigen binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen binding fragment” as used herein include “antibody fragment,” which comprise a portion of an intact antibody, such as the antigen binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or Van der Waals' interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. Lower K_{D} values indicate higher affinity of the antibody. The value of K_{D} varies for different complexes of antibody and antigen and depends on both kon and koff. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment described herein, the terms such as “bind to,” “that specifically bind to,” and analogous terms are also used interchangeably herein and refer to antibodies of antigen binding domains that specifically bind to an antigen, such as a polypeptide. An antibody or antigen binding domain that binds to or specifically binds to an antigen may be cross-reactive with related antigens. In certain embodiments, an antibody or antigen binding domain that binds to or specifically binds to an antigen does not cross-react with other antigens. An antibody or antigen binding domain that binds to or specifically binds to an antigen can be identified, for example, by immunoassays, Octet®, Biacore®, or other techniques known to those of skill in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. With regard to terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding domain that binds to an antigen includes one that is capable of binding the antigen with sufficient affinity such that the antibody or antigen binding fragment is useful, for example, as a diagnostic or therapeutic agent in targeting the antigen. In certain embodiments, an antibody or antigen binding domain that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding domain binds to an epitope of an antigen that is conserved among the antigen from different species (e.g., between human and cynomolgus macaque species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, an Octet®Red96 system, or by Biacore®, using, for example, a Biacore®™-2000 or a Biacore®™-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®™-2000, or the Biacore®™-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos: 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Brüggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et aL, 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus a variable domain (VH) followed by three constant domains (CH) for each of the a and y chains and four CH domains for μ and ϵ isotypes. Each L chain has at the N-terminus a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ϵ), gamma (γ), and mu (μ), based on the amino acid sequence of the heavy chain constant region. The distinct heavy chains differ in size: α, δ, and γ contain approximately 450 amino acids, while μ and ϵ contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are noted below.

TABLE 27

| Loop | Kabat | AbM | Chothia | Contact | IMGT |
|---|---|---|---|---|---|
| CDR L1 | L24--L34 | L24--L34 | L24--L34 | L30--L36 | L27--L38 |
| CDR L2 | L50--L56 | L50--L56 | L50--L56 | L46--L55 | L56--L65 |
| CDR L3 | L89--L97 | L89--L97 | L89--L97 | L89--L96 | L105--L117 |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H52--H56 | H47--H58 | H56--H65 |
| CDR H3 | H95--H102 | H95--H102 | H95--H102 | H93--H101 | H105--H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2”) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, an IL-36α or IL-36γ variant may result from one or more (such as, for example, about 1 to about 25, about 1 to about 20, about 1 to about 15, about 1 to about 10, or about 1 to about 5) changes to an amino acid sequence of a native IL-36α or IL-36γ. Also by way of example, a variant of an anti- IL-36α and/or IL-36γ antibody may result from one or more (such as, for example, about 1 to about 25, about 1 to about 20, about 1 to about 15, about 1 to about 10, or about 1 to about 5) changes to an amino acid sequence of a native or previously unmodified anti- IL-36α and/or IL-36γ antibody. Variants may be naturally occurring, such as allelic or splice variants, or may be artificially constructed. Polypeptide variants may be prepared from the corresponding nucleic acid molecules encoding the variants. In specific embodiments, the IL-36α or IL-36γ variant or anti- IL-36α and/or IL-36γ antibody variant at least retains IL-36α or IL-36γ or anti- IL-36α and/or IL-36γ antibody functional activity, respectively. In specific embodiments, an anti- IL-36α and/or IL-36γ antibody variant binds IL-36α and/or IL-36γ and/or is antagonistic to IL-36α and/or IL-36γ activity. In certain embodiments, the variant is encoded by a single nucleotide polymorphism (SNP) variant of a nucleic acid molecule that encodes IL-36α or IL-36γ or anti- IL-36α and/or IL-36γ antibody VH or VL regions or subregions, such as one or more CDRs.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

As used herein, an “epitope” is a term in the art and refers to a localized region of an antigen to which an antibody or antigen binding fragment can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding an antibody or antigen binding fragment as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody or antigen binding fragment of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antibody or antigen binding fragment, for example, in isolated or purified form, together with a suitable amount of excipients.

The term “effective amount” or “therapeutically effective amount” as used herein refers to the amount of an antibody or antigen binding fragment or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “about” and “approximately” mean within 20%, within 15%, within 10%, within 9%, within 8%, within 7%, within 6%, within 5%, within 4%, within 3%, within 2%, within 1%, or less of a given value or range.

The term “disease or disorder related to skin,” or “disease or disorder related to skin tissue(s)” as used herein refers to any disease or disorder originated or having an impact on any portion of a mammal skin, e.g., human skin. The term “skin” used herein includes any layers of ectodermal tissue, includes any immediate underlying muscles, bones, ligaments and internal organs, and includes both hairy and glabrous skin (hairless). The disease or disorder related to skin tissue can be but not limited to a disease or disorder originated or affecting epidermis, superficial arteriovenous plexus, papillar dermis, dermis including reticular dermis, meissner's corpuscle, sweat duct, and subcutis or hypodermis including deep arteriovenous plexus and subcutaneous fat. The term “disease or disorder related to intestinal tissue(s)” as used herein refers to any disease or disorder originated or having impact on any portion of a mammal intestine, e.g., human intestine. The term “disease or disorder related to lung tissue(s)” as used herein refers to any disease or disorder originated or having impact on any portion of a mammal lung, e.g., human lung.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Anti-IL-36 Antibodies and Related Molecules

### 5.2.1 Anti-IL-36 Antibodies

The antibodies provided herein include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In particular, the antibodies provided herein include immunoglobulin molecules and immunologically active portions of immunoglobulin molecules, i.e., molecules that contain an antigen binding site that immunospecifically binds to an IL-36α and/or IL-36γ antigen. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein is an IgG antibody, such as an IgG1, IgG2 or IgG4 antibody.

Variants and derivatives of antibodies include antibody fragments that retain the ability to specifically bind to an epitope. Exemplary fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as, a scFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific scFv (a scFv or a dsFv molecule having two antigen-binding domains, each of which may be directed to a different epitope); a diabody (a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (a trimerized scFv, formed in a manner similar to a diabody, but in which three antigen-binding domains are created in a single complex; the three antigen binding domains may be directed towards the same or different epitopes); and a tetrabody (a tetramerized scFv, formed in a manner similar to a diabody, but in which four antigen-binding domains are created in a single complex; the four antigen binding domains may be directed towards the same or different epitopes). Derivatives of antibodies also include one or more CDR sequences of an antibody combining site. The CDR sequences may be linked together on a scaffold when two or more CDR sequences are present. In certain embodiments, an antibody provided herein comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, N.Y., pp. 269-315 (1994).

The antibodies provided herein may be from any animal origin including birds and mammals (e.g., human, monkey, murine, donkey, sheep, rabbit, goat, guinea pig, camel, horse, or chicken). In certain embodiments, the antibodies provided herein are human or humanized monoclonal antibodies. As used herein, “human” antibodies include antibodies having the amino acid sequence of a human immunoglobulin and include antibodies isolated from human immunoglobulin libraries or from mice that express antibodies from human genes.

In certain embodments, the antibodies are full mouse antibodies. In certain embodiments, the antibodies are humanized antibodies. In certain embodiments, the antibodies are fully human antibodies, such as fully human antibodies that immunospecifically bind an IL-36α and/or IL-36γ polypeptide, an IL-36α and/or IL-36γ polypeptide fragment, or an IL-36α and/or IL-36γ epitope.

The antibodies provided herein may be monospecific, bispecific, trispecific or of greater multispecificity. For example, in certain embodiments, the bispecific antibodies have one specificity to one epitope of an IL-36α and/or IL-36γ polypeptide and a second specificity to a second epitope of the IL-36α and/or IL-36γ polypeptide. In other embodiments, the bispecific antobidies have one specificity to an IL-36α and/or IL-36γ polypeptide and a second specificity for a heterologous epitope, such as a heterologous polypeptide or solid support material.

In some embodiments, provided herein are antibodies that bind to IL-36α and/or IL-36γ. In certain embodiments, the antibodies provided herein bind to both IL-36α and IL-36γ. In certain embodiments, the antibodies provided herein bind to human IL-36α and IL-36γ. In other embodiments, the antibodies provided herein bind to cynomolgus macaque IL-36α and IL-36γ. In yet other embodiments, the antibodies provided herein binds to both human IL-36α and IL-36γ and cynomolgus macaque IL-36α and IL-36γ. In some embodiments, the antibodies provided herein do not bind to human or cynomolgus macaque IL-36β. In some embodiments, the antibodies provided herein do not block the binding of an IL-36β to IL-36 receptor. In some embodiments, the antibodies provided herein do not bind to human or cynomolgus macaque IL-36Ra. In some embodiments, the antibodies provided herein do not block the binding of an IL-36Ra to IL-36 receptor. In some embodiments, the antibodies provided herein do not bind to human or cynomolgus macaque IL-36β and IL-36Ra. In some embodiments, the antibodies provided herein do not block the binding of IL-36β and IL-36Ra to IL-36 receptor.

In other embodiments, the antibodies provided herein are humanized antibodies (e.g., comprising human constant and framework regions) that bind IL-36α and IL-36γ, including an IL-36α and/or IL-36γ polypeptide, an IL-36α and/or IL-36γ polypeptide fragment, an IL-36α and/or IL-36γ peptide, or an IL-36α and/or IL-36γ epitope.

The terms “IL-36α” and “IL-36α polypeptide” encompasses a polypeptide (“polypeptide” and “protein” are used interchangeably herein), including any native polypeptide, from any vertebrate source, including mammals such as primates (e.g., humans and cynomolgus monkeys (cynomolgus macaque)), dogs, and rodents (e.g., mice and rats), unless otherwise indicated. The term “IL-36α” also encompasses “full-length,” unprocessed IL-36α as well as any form of IL-36α that results from processing in the cell or extracellularly. “Related IL-36α polypeptides” include allelic variants (e.g., SNP variants); splice variants; fragments; derivatives; substitution, deletion, and insertion variants; fusion polypeptides; interspecies homologs; and interspecies chimeras, which can retain IL-36α activity. As those skilled in the art will appreciate, an anti- IL-36α antibody provided herein can bind to an IL-36α polypeptide, an IL-36α polypeptide fragment, an IL-36α antigen, and/or an IL-36α epitope. An “epitope” may be part of a larger IL-36α antigen, which may be part of a larger IL-36α polypeptide fragment, which, in turn, may be part of a larger IL-36α polypeptide. IL-36α may exist in a native or denatured form. IL-36α polypeptides described herein may be isolated from a variety of sources, such as from human tissue types or from another source, or prepared by recombinant or synthetic methods. Orthologs to the IL-36α polypeptide are also well known in the art.

In some embodiments, the human IL-36α has an amino acid sequence of SEQ ID NO: 1 (GenBank™ accession number NP_055255.1) as provided below:

(SEQ ID NO: 1)
MEKALKIDTPQQGSIQDINHRVWVLQDQTLIAVPRKDRMSPVTIALISCRH
VETLEKDRGNPIYLGLNGLNLCLMCAKVGDQPTLQLKEKDIMDLYNQPEPV
KSFLFYHSQSGRNSTFESVAFPGWFIAVSSEGGCPLILTQELGKANTTDFG
LTMLF.

The corresponding encoding nucleic acid sequence of the above human IL-36α protein has a polynucleotide sequence of SEQ ID NO: 99 as provided below:

(SEQ ID NO: 99)
ATGGAAAAAGCATTGAAAATTGACACACCTCAGCAGGGGAGCATTCAGGAT
ATCAATCATCGGGTGTGGGTTCTTCAGGACCAGACGCTCATAGCAGTCCCG
AGGAAGGACCGTATGTCTCCAGTCACTATTGCCTTAATCTCATGCCGACAT
GTGGAGACCCTTGAGAAAGACAGAGGGAACCCCATCTACCTGGGCCTGAAT
GGACTCAATCTCTGCCTGATGTGTGCTAAAGTCGGGGACCAGCCCACACTG
CAGCTGAAGGAAAAGGATATAATGGATTTGTACAACCAACCCGAGCCTGTG
AAGTCCTTTCTCTTCTACCACAGCCAGAGTGGCAGGAACTCCACCTTCGAG
TCTGTGGCTTTCCCTGGCTGGTTCATCGCTGTCAGCTCTGAAGGAGGCTGT
CCTCTCATCCTTACCCAAGAACTGGGGAAAGCCAACACTACTGACTTTGGG
TTAACTATGCTGTTT.

In some embodiments, the human IL-36α has an amino acid sequence of SEQ ID NO: 101 as provided below:

(SEQ ID NO: 101)
MEKALKIDTPQRGSIQDINHRVWVLQDQTLIAVPRKDRMSPVTIALISCRH
VETLEKDRGNPIYLGLNGLNLCLMCAKVGDQPTLQLKEKDIMDLYNQPEPV
KSFLFYHSQSGRNSTFESVAFPGWFIAVSSEGGCPLILTQELGKANTTDFG
LTMLF.

The corresponding encoding nucleic acid sequence of the above human IL-36α protein has a polynucleotide sequence of SEQ ID NO: 100 as provided below:

(SEQ ID NO: 100)
ATGGAAAAAGCATTGAAAATTGACACACCTCAGCGGGGGAGCATTCAGGAT
ATCAATCATCGGGTGTGGGTTCTTCAGGACCAGACGCTCATAGCAGTCCCG
AGGAAGGACCGTATGTCTCCAGTCACTATTGCCTTAATCTCATGCCGACAT
GTGGAGACCCTTGAGAAAGACAGAGGGAACCCCATCTACCTGGGCCTGAAT
GGACTCAATCTCTGCCTGATGTGTGCTAAAGTCGGGGACCAGCCCACACTG
CAGCTGAAGGAAAAGGATATAATGGATTTGTACAACCAACCCGAGCCTGTG
AAGTCCTTTCTCTTCTACCACAGCCAGAGTGGCAGGAACTCCACCTTCGAG
TCTGTGGCTTTCCCTGGCTGGTTCATCGCTGTCAGCTCTGAAGGAGGCTGT
CCTCTCATCCTTACCCAAGAACTGGGGAAAGCCAACACTACTGACTTTGGG
TTAACTATGCTGTTT.

In some embodiments, the human IL-36α (a truncated variant) has an amino acid sequence of SEQ ID NO:5 (translation of dbSNP:rs895497 of NCBI GenBank™ accession number NM_014440) as provided below:

(SEQ ID NO: 5)
KIDTPQRGSIQDINHRVWVLQDQTLIAVPRKDRMSPVTIALISCRHVETLE
KDRGNPIYLGLNGLNLCLMCAKVGDQPTLQLKEKDIMDLYNQPEPVKSFLF
YHSQSGRNSTFESVAFPGWFIAVSSEGGCPLILTQELGKANTTDFGLTML
F.

The corresponding encoding nucleic acid sequence of the above human IL-36α protein has a polynucleotide sequence of SEQ ID NO: 4 (dbSNP:rs895497 of NCBI GenBank™ accession number NM_014440) as provided below:

(SEQ ID NO: 4)
AAAATTGACACACCTCAGCGGGGGAGCATTCAGGATATCAATCATCGGGTG
TGGGTTCTTCAGGACCAGACGCTCATAGCAGTCCCGAGGAAGGACCGTATG
TCTCCAGTCACTATTGCCTTAATCTCATGCCGACATGTGGAGACCCTTGAG
AAAGACAGAGGGAACCCCATCTACCTGGGCCTGAATGGACTCAATCTCTGC
CTGATGTGTGCTAAAGTCGGGGACCAGCCCACACTGCAGCTGAAGGAAAAG
GATATAATGGATTTGTACAACCAACCCGAGCCTGTGAAGTCCTTTCTCTTC
TACCACAGCCAGAGTGGCAGGAACTCCACCTTCGAGTCTGTGGCTTTCCCT
GGCTGGTTCATCGCTGTCAGCTCTGAAGGAGGCTGTCCTCTCATCCTTACC
CAAGAACTGGGGAAAGCCAACACTACTGACTTTGGGTTAACTATGCTGTTT
TAA.

In other embodiments, the human IL-36α (a truncated variant) has an amino acid sequence of SEQ ID NO: 7 (GenBank™ accession number NP_055255.1) as provided below:

(SEQ ID NO: 7)
KIDTPQQGSIQDINHRVWVLQDQTLIAVPRKDRMSPVTIALISCRHVETLE
KDRGNPIYLGLNGLNLCLMCAKVGDQPTLQLKEKDIMDLYNQPEPVKSFLF
YHSQSGRNSTFESVAFPGWFIAVSSEGGCPLILTQELGKANTTDFGLTML
F.

The corresponding encoding nucleic acid sequence of the above human IL-36α protein has a polynucleotide sequence of SEQ ID NO: 6 (GenBank™ accession number NM_014440.1) as provided below:

(SEQ ID NO: 6)
AAAATTGACACACCTCAGCAGGGGAGCATTCAGGATATCAATCATCGGGTG
TGGGTTCTTCAGGACCAGACGCTCATAGCAGTCCCGAGGAAGGACCGTATG
TCTCCAGTCACTATTGCCTTAATCTCATGCCGACATGTGGAGACCCTTGAG
AAAGACAGAGGGAACCCCATCTACCTGGGCCTGAATGGACTCAATCTCTGC
CTGATGTGTGCTAAAGTCGGGGACCAGCCCACACTGCAGCTGAAGGAAAAG
GATATAATGGATTTGTACAACCAACCCGAGCCTGTGAAGTCCTTTCTCTTC
TACCACAGCCAGAGTGGCAGGAACTCCACCTTCGAGTCTGTGGCTTTCCCT
GGCTGGTTCATCGCTGTCAGCTCTGAAGGAGGCTGTCCTCTCATCCTTACC
CAAGAACTGGGGAAAGCCAACACTACTGACTTTGGGTTAACTATGCTGTTT
TAA.

In certain embodiments, the cynomolgus macaque IL-36α has an amino acid sequence of SEQ ID NO: 109 as provided below:

(SEQ ID NO: 109)
MKKFIVVLYGKLRLCSWSLSELFSMSKSEMPQPVSIQDINHRVWVLQDQIL
IAVPRKDRVSPVTISLISCRHVETLEKDRGNPIYLGLNGLNLCLMCAKAGD
QPTLQLKEKDIMDLYNQPEPVKSFLFYHSQSGRNSTFESVAFPGWFIAVSS
EGGCPLILTQELGKANTTDFGLTMLF.

The corresponding encoding nucleic acid sequence of the above cynomolgus macaque IL-36α protein has a polynucleotide sequence of SEQ ID NO: 108 as provided below:

(SEQ ID NO: 108)
ATGAAAAAATTCATTGTTGTACTATATGGAAAACTCAGGCTGTGTTCATGG
TCTTTGAGTGAACTATTTTCAATGTCGAAAAGTGAAATGCCTCAGCCGGTG
AGCATTCAGGATATCAATCATCGGGTGTGGGTTCTTCAGGACCAGATCCTC
ATAGCAGTCCCGAGGAAGGACCGTGTGTCTCCAGTCACTATTTCCTTAATC
TCATGCCGACATGTGGAGACCCTTGAGAAAGACAGAGGGAACCCCATCTAC
CTGGGACTGAATGGGCTCAATCTCTGCTTGATGTGTGCTAAGGCCGGGGAC
CAGCCCACACTGCAGCTGAAGGAAAAGGATATAATGGATTTGTACAACCAA
CCTGAGCCTGTGAAGTCCTTTCTCTTCTACCACAGCCAGAGTGGCAGGAAC
TCCACCTTCGAGTCTGTGGCCTTCCCTGGCTGGTTCATTGCTGTCAGCTCT
GAAGGAGGCTGTCCTCTCATCCTTACCCAAGAACTGGGGAAAGCCAACACT
ACTGACTTTGGGTTAACTATGCTGTTT.

In certain embodiments, the cynomolgus macaque IL-36α (a truncated variant in Macaca fascicularis) has an amino acid sequence of SEQ ID NO: 13 (XP_015288898.1) as provided below:

(SEQ ID NO: 13)
KSEMPQPVSIQDINHRVWVLQDQILIAVPRKDRVSPVTISLISCRHVETLE
KDRGNPIYLGLNGLNLCLMCAKAGDQPTLQLKEKDIMDLYNQPEPVKSFLF
YHSQSGRNSTFESVAFPGWFIAVSSEGGCPLILTQELGKANTTDFGLTMLF

The corresponding encoding nucleic acid sequence of the above cynomolgus macaque IL-36α protein has a polynucleotide sequence of SEQ ID NO: 12 (XM_015433412) as provided below:

(SEQ ID NO: 12)
AAAAGTGAAATGCCTCAGCCGGTGAGCATTCAGGATATCAATCATCGGGTG
TGGGTTCTTCAGGACCAGATCCTCATAGCAGTCCCGAGGAAGGACCGTGTG
TCTCCAGTCACTATTTCCTTAATCTCATGCCGACATGTGGAGACCCTTGAG
AAAGACAGAGGGAACCCCATCTACCTGGGACTGAATGGGCTCAATCTCTGC
TTGATGTGTGCTAAGGCCGGGGACCAGCCCACACTGCAGCTGAAGGAAAAG
GATATAATGGATTTGTACAACCAACCTGAGCCTGTGAAGTCCTTTCTCTTC
TACCACAGCCAGAGTGGCAGGAACTCCACCTTCGAGTCTGTGGCCTTCCCT
GGCTGGTTCATTGCTGTCAGCTCTGAAGGAGGCTGTCCTCTCATCCTTACC
CAAGAACTGGGGAAAGCCAACACTACTGACTTTGGGTTAACTATGCTGTTT
TAA.

The terms “IL-36γ” and “IL-36γ polypeptide” encompasses a polypeptide (“polypeptide” and “protein” are used interchangeably herein), including any native polypeptide, from any vertebrate source, including mammals such as primates (e.g., humans and cynomolgus monkeys (cynomolgus macaque)), dogs, and rodents (e.g., mice and rats), unless otherwise indicated. The term “IL-36γ” also encompasses “full-length,” unprocessed IL-36γ as well as any form of IL-36γ that results from processing in the cell or extracellularly. “Related IL-36γ polypeptides” include allelic variants (e.g., SNP variants); splice variants; fragments; derivatives; substitution, deletion, and insertion variants; fusion polypeptides; interspecies homologs; and interspecies chimeras, which can retain IL-36γ activity. As those skilled in the art will appreciate, an anti- IL-36γ antibody provided herein can bind to an IL-36γ polypeptide, an IL-36γ polypeptide fragment, an IL-36γ antigen, and/or an IL-36γ epitope. An “epitope” may be part of a larger IL-36γ antigen, which may be part of a larger IL-36γ polypeptide fragment, which, in turn, may be part of a larger IL-36γ polypeptide. IL-36γ may exist in a native or denatured form. IL-36γ polypeptides described herein may be isolated from a variety of sources, such as from human tissue types or from another source, or prepared by recombinant or synthetic methods. Orthologs to the IL-36γ polypeptide are also well known in the art.

In some embodiments, the human IL-36γ has an amino acid sequence of SEQ ID NO: 3 (GenBank™ accession number NP_062564.1) as provided below:

(SEQ ID NO: 3)
MRGTPGDADGGGRAVYQSMCKPITGTINDLNQQVWTLQGQNLVAVPRSDSV
TPVTVAVITCKYPEALEQGRGDPIYLGIQNPEMCLYCEKVGEQPTLQLKEQ
KIMDLYGQPEPVKPFLFYRAKTGRTSTLESVAFPDWFIASSKRDQPIILTS
ELGKSYNTAFELNIND.

The corresponding encoding nucleic acid sequence of the above human IL-36γ protein has a polynucleotide sequence of SEQ ID NO: 103 as provided below:

(SEQ ID NO: 103)
ATGAGAGGCACTCCAGGAGACGCTGATGGTGGAGGAAGGGCCGTCTATCAA
TCAATGTGTAAACCTATTACTGGGACTATTAATGATTTGAATCAGCAAGTG
TGGACCCTTCAGGGTCAGAACCTTGTGGCAGTTCCACGAAGTGACAGTGTG
ACCCCAGTCACTGTTGCTGTTATCACATGCAAGTATCCAGAGGCTCTTGAG
CAAGGCAGAGGGGATCCCATTTATTTGGGAATCCAGAATCCAGAAATGTGT
TTGTATTGTGAGAAGGTTGGAGAACAGCCCACATTGCAGCTAAAAGAGCAG
AAGATCATGGATCTGTATGGCCAACCCGAGCCCGTGAAACCCTTCCTTTTC
TACCGTGCCAAGACTGGTAGGACCTCCACCCTTGAGTCTGTGGCCTTCCCG
GACTGGTTCATTGCCTCCTCCAAGAGAGACCAGCCCATCATTCTGACTTCA
GAACTTGGGAAGTCATACAACACTGCCTTTGAATTAAATATAAATGAC.

In some embodiments, the human IL-36γ (a truncated variant) has an amino acid sequence of SEQ ID NO: 10 (a truncated version of GenBank™ accession number NP_062564.1) as provided below:

(SEQ ID NO: 10)
SMCKPITGTINDLNQQVWTLQGQNLVAVPRSDSVTPVTVAVITCKYPEALE
QGRGDPIYLGIQNPEMCLYCEKVGEQPTLQLKEQKIMDLYGQPEPVKPFLF
YRAKTGRTSTLESVAFPDWFIASSKRDQPIILTSELGKSYNTAFELNIND.

The corresponding encoding nucleic acid sequence of the above human IL-36γ protein has a polynucleotide sequence of SEQ ID NO: 104 as provided below:

(SEQ ID NO: 104)
TCAATGTGTAAACCTATTACTGGGACTATTAATGATTTGAATCAGCAAGTG
TGGACCCTTCAGGGTCAGAACCTTGTGGCAGTTCCACGAAGTGACAGTGTG
ACCCCAGTCACTGTTGCTGTTATCACATGCAAGTATCCAGAGGCTCTTGAG
CAAGGCAGAGGGGATCCCATTTATTTGGGAATCCAGAATCCAGAAATGTGT
TTGTATTGTGAGAAGGTTGGAGAACAGCCCACATTGCAGCTAAAAGAGCAG
AAGATCATGGATCTGTATGGCCAACCCGAGCCCGTGAAACCCTTCCTTTTC
TACCGTGCCAAGACTGGTAGGACCTCCACCCTTGAGTCTGTGGCCTTCCCG
GACTGGTTCATTGCCTCCTCCAAGAGAGACCAGCCCATCATTCTGACTTCA
GAACTTGGGAAGTCATACAACACTGCCTTTGAATTAAATATAAATGAC.

In certain embodiments, the cynomolgus macaque IL-36γ has an amino acid sequence of SEQ ID NO: 113 (XP_015288884) as provided below:

(SEQ ID NO: 113)
MRGTPGNPAGGGRVVYQSMRTPITGTINDLNQQVWTLQGQILVAVPRSDSV
TPVTVAVITCKYPEALDQSRGDPIYLGIRNPEMCLCCEEVGGQPTLQLKEQ
KIMDLYGQPEPVKPFLFYRVKTGRTSTLESVAFPNWFIASSTRDQPIILTS
ELGKSYNTAFELNIK.

The corresponding encoding nucleic acid sequence of the above cynomolgus macaque IL-36γ protein has a polynucleotide sequence of SEQ ID NO: 112 (XM_015433398) as provided below:

(SEQ ID NO: 112)
ATGAGAGGCACTCCAGGAAACCCTGCTGGTGGAGGAAGGGTCGTCTATCAG
TCAATGCGTACACCTATTACTGGGACTATTAATGATTTGAATCAGCAAGTG
TGGACCCTTCAGGGTCAGATCCTTGTGGCAGTTCCACGAAGTGACAGTGTG
ACCCCAGTCACTGTCGCTGTTATCACATGCAAGTATCCAGAGGCTCTTGAC
CAAAGCAGAGGGGATCCCATTTATTTGGGAATCCGGAATCCAGAAATGTGT
TTGTGTTGTGAGGAGGTTGGAGGACAGCCCACGTTGCAGCTAAAAGAGCAG
AAGATCATGGATTTGTATGGCCAGCCCGAGCCTGTGAAACCCTTCCTTTTC
TACCGTGTCAAGACCGGTAGGACCTCCACCCTTGAGTCTGTGGCCTTCCCA
AACTGGTTCATTGCCTCTTCCACGAGAGACCAGCCCATCATCCTGACTTCA
GAACTTGGGAAGTCATACAACACTGCCTTTGAATTAAATATAAAA.

In certain embodiments, the cynomolgus macaque IL-36γ (a truncated variant in Macaca fascicularis) has an amino acid sequence of SEQ ID NO: 17 (XP_015288884) as provided below:

(SEQ ID NO: 17)
SMRTPITGTINDLNQQVWTLQGQILVAVPRSDSVTPVTVAVITCKYPEALD
QSRGDPIYLGIRNPEMCLCCEEVGGQPTLQLKEQKIMDLYGQPEPVKPFLF
YRVKTGRTSTLESVAFPNWFIASSTRDQPIILTSELGKSYNTAFELNIK

The corresponding encoding nucleic acid sequence of the above cynomolgus macaque IL-36γ protein has a polynucleotide sequence of SEQ ID NO: 16 (XM_015433398) as provided below:

(SEQ ID NO: 16)
TCAATGCGTACACCTATTACTGGGACTATTAATGATTTGAATCAGCAAGTG
TGGACCCTTCAGGGTCAGATCCTTGTGGCAGTTCCACGAAGTGACAGTGTG
ACCCCAGTCACTGTCGCTGTTATCACATGCAAGTATCCAGAGGCTCTTGAC
CAAAGCAGAGGGGATCCCATTTATTTGGGAATCCGGAATCCAGAAATGTGT
TTGTGTTGTGAGGAGGTTGGAGGACAGCCCACGTTGCAGCTAAAAGAGCAG
AAGATCATGGATTTGTATGGCCAGCCCGAGCCTGTGAAACCCTTCCTTTTC
TACCGTGTCAAGACCGGTAGGACCTCCACCCTTGAGTCTGTGGCCTTCCCA
AACTGGTTCATTGCCTCTTCCACGAGAGACCAGCCCATCATCCTGACTTCA
GAACTTGGGAAGTCATACAACACTGCCTTTGAATTAAATATAAAATAA.

The terms “IL-36β” and “IL-36β polypeptide” encompasses a polypeptide (“polypeptide” and “protein” are used interchangeably herein), including any native polypeptide, from any vertebrate source, including mammals such as primates (e.g., humans and cynomolgus monkeys (cynomolgus macaque)), dogs, and rodents (e.g., mice and rats), unless otherwise indicated. The term “IL-36β” also encompasses “full-length,” unprocessed IL-36β as well as any form of IL-36β that results from processing in the cell or extracellularly. “Related IL-36β polypeptides” include allelic variants (e.g., SNP variants); splice variants; fragments; derivatives; substitution, deletion, and insertion variants; fusion polypeptides; interspecies homologs; and interspecies chimeras, which can retain IL-36β activity. IL-36β may exist in a native or denatured form. IL-36β polypeptides described herein may be isolated from a variety of sources, such as from human tissue types or from another source, or prepared by recombinant or synthetic methods. Orthologs to the IL-36β polypeptide are also well known in the art.

In some embodiments, the human IL-36β has an amino acid sequence of SEQ ID NO: 2 (GenBank™ accession number NP_775270.1) as provided below:

(SEQ ID NO: 2)
MNPQREAAPKSYAIRDSRQMVWVLSGNSLIAAPLSRSIKPVTLHLIACRDT
EFSDKEKGNMVYLGIKGKDLCLFCAEIQGKPTLQLKEKNIMDLYVEKKAQK
PFLFFHNKEGSTSVFQSVSYPGWFIATSTTSGQPIFLTKERGITNNTNFYL
DSVE.

The corresponding encoding nucleic acid sequence of the above human IL-36β has a polynucleotide sequence of SEQ ID NO: 102 as provided below:

(SEQ ID NO: 102)
ATGAACCCACAACGGGAGGCAGCACCCAAATCCTATGCTATTCGTGATTCT
CGACAGATGGTGTGGGTCCTGAGTGGAAATTCTTTAATAGCAGCTCCTCTT
AGCCGCAGCATTAAGCCTGTCACTCTTCATTTAATAGCCTGTAGAGACACA
GAATTCAGTGACAAGGAAAAGGGTAATATGGTTTACCTGGGAATCAAGGGA
AAAGATCTCTGTCTCTTCTGTGCAGAAATTCAGGGCAAGCCTACTTTGCAG
CTTAAGGAAAAAAATATCATGGACCTGTATGTGGAGAAGAAAGCACAGAAG
CCCTTTCTCTTTTTCCACAATAAAGAAGGCTCCACTTCTGTCTTTCAGTCA
GTCTCTTACCCTGGCTGGTTCATAGCCACCTCCACCACATCAGGACAGCCC
ATCTTTCTCACCAAGGAGAGAGGCATAACTAATAACACTAACTTCTACTTA
GATTCTGTGGAA.

In some embodiments, the human IL-36β (a truncated variant) has an amino acid sequence of SEQ ID NO: 9 (a truncated version of GenBank™ accession number NP_775270.1) as provided below:

(SEQ ID NO: 9)
REAAPKSYAIRDSRQMVWVLSGNSLIAAPLSRSIKPVTLHLIACRDTEFSD
KEKGNMVYLGIKGKDLCLFCAEIQGKPTLQLKEKNIMDLYVEKKAQKPFLF
FHNKEGSTSVFQSVSYPGWFIATSTTSGQPIFLTKERGITNNTNFYLDSV
E.

The corresponding encoding nucleic acid sequence of the above human IL-36β protein has a polynucleotide sequence of SEQ ID NO: 8 (a truncated version of GenBank™ accession number NM_173178) as provided below:

(SEQ ID NO: 8)
CGGGAGGCAGCACCCAAATCCTATGCTATTCGTGATTCTCGACAGATGGTG
TGGGTCCTGAGTGGAAATTCTTTAATAGCAGCTCCTCTTAGCCGCAGCATT
AAGCCTGTCACTCTTCATTTAATAGCCTGTAGAGACACAGAATTCAGTGAC
AAGGAAAAGGGTAATATGGTTTACCTGGGAATCAAGGGAAAAGATCTCTGT
CTCTTCTGTGCAGAAATTCAGGGCAAGCCTACTTTGCAGCTTAAGGAAAAA
AATATCATGGACCTGTATGTGGAGAAGAAAGCACAGAAGCCCTTTCTCTTT
TTCCACAATAAAGAAGGCTCCACTTCTGTCTTTCAGTCAGTCTCTTACCCT
GGCTGGTTCATAGCCACCTCCACCACATCAGGACAGCCCATCTTTCTCACC
AAGGAGAGAGGCATAACTAATAACACTAACTTCTACTTAGATTCTGTGGAA
TAA.

In certain embodiments, the cynomolgus macaque IL-36β has an amino acid sequence of SEQ ID NO: 111 as provided below:

(SEQ ID NO: 111)
MNPQWQAAPKSYAIRDSRQMVWVLSGNSLIAAPLSNRVKPVTLHLITCRDT
EFSDKKKGNLVYLGIRGKDLCLFCEEIQGKPTLQLKEKNIMDLYMEKKAQK
PFLFFHNKEGSSSVFQSVSYPGWFIATSSTSGQPIFLTQERGITNNTNFYL
DSVE.

The corresponding encoding nucleic acid sequence of the above cynomolgus macaque IL-36β protein has a polynucleotide sequence of SEQ ID NO: 110 as provided below:

(SEQ ID NO: 110)
ATGAACCCACAATGGCAGGCAGCACCCAAATCCTATGCTATTCGTGATTCT
CGACAGATGGTGTGGGTCCTGAGTGGAAATTCTTTAATAGCAGCTCCTCTT
AGCAACCGTGTTAAGCCTGTCACTCTTCATTTAATAACCTGCAGAGACACA
GAATTCAGTGATAAGAAAAAGGGTAATCTGGTTTACCTGGGAATCAGGGGA
AAAGATCTCTGTCTCTTCTGTGAAGAAATTCAGGGCAAACCTACTTTGCAG
CTTAAGGAGAAAAACATCATGGACCTGTACATGGAGAAGAAAGCACAGAAG
CCCTTTCTCTTTTTCCACAATAAAGAAGGCTCCAGTTCTGTCTTTCAGTCA
GTCTCTTACCCTGGCTGGTTCATAGCCACCTCCTCCACATCAGGACAGCCC
ATCTTTCTCACCCAGGAGAGGGGCATAACTAACAACACTAACTTCTACTTA
GATTCTGTGGAA.

In certain embodiments, the cynomolgus macaque IL-36β (a truncated variant in Macaca fascicularis) has an amino acid sequence of SEQ ID NO: 15 (XP_005575353) as provided below:

(SEQ ID NO: 15)
WQAAPKSYAIRDSRQMVWVLSGNSLIAAPLSNRVKPVTLHLITCRDTEFSD
KKKGNLVYLGIRGKDLCLFCEEIQGKPTLQLKEKNIMDLYMEKKAQKPFLF
FHNKEGSSSVFQSVSYPGWFIATSSTSGQPIFLTQERGITNNTNFYLDSVE

The corresponding encoding nucleic acid sequence of the above cynomolgus macaque IL-36β protein has a polynucleotide sequence of SEQ ID NO: 14 (XM_005575296) as provided below:

(SEQ ID NO: 14)
TGGCAGGCAGCACCCAAATCCTATGCTATTCGTGATTCTCGACAGATGGTG
TGGGTCCTGAGTGGAAATTCTTTAATAGCAGCTCCTCTTAGCAACCGTGTT
AAGCCTGTCACTCTTCATTTAATAACCTGCAGAGACACAGAATTCAGTGAT
AAGAAAAAGGGTAATCTGGTTTACCTGGGAATCAGGGGAAAAGATCTCTGT
CTCTTCTGTGAAGAAATTCAGGGCAAACCTACTTTGCAGCTTAAGGAGAAA
AACATCATGGACCTGTACATGGAGAAGAAAGCACAGAAGCCCTTTCTCTTT
TTCCACAATAAAGAAGGCTCCAGTTCTGTCTTTCAGTCAGTCTCTTACCCT
GGCTGGTTCATAGCCACCTCCTCCACATCAGGACAGCCCATCTTTCTCACC
CAGGAGAGGGGCATAACTAACAACACTAACTTCTACTTAGATTCTGTGGAA
TAA.

The terms “IL-36Ra” and “IL-36Ra polypeptide” encompasses a polypeptide (“polypeptide” and “protein” are used interchangeably herein), including any native polypeptide, from any vertebrate source, including mammals such as primates (e.g., humans and cynomolgus monkeys (cynomolgus macaque)), dogs, and rodents (e.g., mice and rats), unless otherwise indicated. The term “IL-36Ra” also encompasses “full-length,” unprocessed IL-36Ra as well as any form of IL-36Ra that results from processing in the cell or extracellularly. “Related IL-36Ra polypeptides” include allelic variants (e.g., SNP variants); splice variants; fragments; derivatives; substitution, deletion, and insertion variants; fusion polypeptides; interspecies homologs; and interspecies chimeras, which can retain IL-36Ra activity. IL-36Ra may exist in a native or denatured form. IL-36Ra polypeptides described herein may be isolated from a variety of sources, such as from human tissue types or from another source, or prepared by recombinant or synthetic methods. Orthologs to the IL-36Ra polypeptide are also well known in the art.

In some embodiments, the human IL-36Ra has an amino acid sequence of SEQ ID NO: 106 as provided below:

(SEQ ID NO: 106)
MVLSGALCFRMKDSALKVLYLHNNQLLAGGLHAGKVIKGEEISVVPNRWLD
ASLSPVILGVQGGSQCLSCGVGQEPTLTLEPVNIMELYLGAKESKSFTFYR
RDMGLTSSFESAAYPGWFLCTVPEADQPVRLTQLPENGGWNAPITDFYFQQ
CD.

The corresponding encoding nucleic acid sequence of the above human IL-36Ra has a polynucleotide sequence of SEQ ID NO: 105 as provided below:

(SEQ ID NO: 105)
ATGGTCCTGAGTGGGGCGCTGTGCTTCCGAATGAAGGACTCGGCATTGAAG
GTGCTTTATCTGCATAATAACCAGCTTCTAGCTGGAGGGCTGCATGCAGGG
AAGGTCATTAAAGGTGAAGAGATCAGCGTGGTCCCCAATCGGTGGCTGGAT
GCCAGCCTGTCCCCCGTCATCCTGGGTGTCCAGGGTGGAAGCCAGTGCCTG
TCATGTGGGGTGGGGCAGGAGCCGACTCTAACACTAGAGCCAGTGAACATC
ATGGAGCTCTATCTTGGTGCCAAGGAATCCAAGAGCTTCACCTTCTACCGG
CGGGACATGGGGCTCACCTCCAGCTTCGAGTCGGCTGCCTACCCGGGCTGG
TTCCTGTGCACGGTGCCTGAAGCCGATCAGCCTGTCAGACTCACCCAGCTT
CCCGAGAATGGTGGCTGGAATGCCCCCATCACAGACTTCTACTTCCAGCAG
TGTGAC.

In some embodiments, the human IL-36Ra (a truncated variant) has an amino acid sequence of SEQ ID NO: 11 (GenBank™ accession number NP_036407, UniProt accession number Q9UBH0) as provided below:

(SEQ ID NO: 11)
VLSGALCFRMKDSALKVLYLHNNQLLAGGLHAGKV
IKGEEISVVPNRWLDASLSPVILGVQGGSQCLSCG
VGQEPTLTLEPVNIMELYLGAKESKSFTFYRRDMG
LTSSFESAAYPGWFLCTVPEADQPVRLTQLPENGG
WNAPITDFYFQQCD.

The corresponding encoding nucleic acid sequence of the above human IL-36Ra has a polynucleotide sequence of SEQ ID NO: 107 as provided below:

(SEQ ID NO: 107)
GTCCTGAGTGGGGCGCTGTGCTTCCGAATGAAGGA
CTCGGCATTGAAGGTGCTTTATCTGCATAATAACC
AGCTTCTAGCTGGAGGGCTGCATGCAGGGAAGGTC
ATTAAAGGTGAAGAGATCAGCGTGGTCCCCAATCG
GTGGCTGGATGCCAGCCTGTCCCCCGTCATCCTGG
GTGTCCAGGGTGGAAGCCAGTGCCTGTCATGTGGG
GTGGGGCAGGAGCCGACTCTAACACTAGAGCCAGT
GAACATCATGGAGCTCTATCTTGGTGCCAAGGAAT
CCAAGAGCTTCACCTTCTACCGGCGGGACATGGGG
CTCACCTCCAGCTTCGAGTCGGCTGCCTACCCGGG
CTGGTTCCTGTGCACGGTGCCTGAAGCCGATCAGC
CTGTCAGACTCACCCAGCTTCCCGAGAATGGTGGC
TGGAATGCCCCCATCACAGACTTCTACTTCCAGCA
GTGTGAC.

In some embodiments, the antibody or fragment thereof provided herein binds to one or more amino acid residues selected from the 45th amino acid residue to the 100th amino acid residue of the amino acid sequence of IL-36α represented by SEQ ID NO: 5 or SEQ ID NO: 7. In some embodiments, the antibody or fragment thereof provided herein binds to one or more amino acid residues selected from the 45th amino acid residue to the 100th amino acid residue of the amino acid sequence of IL-36γ represented by SEQ ID NO: 10. In some embodiments, the antibody or fragment thereof provided herein binds to 2, 3, 4, 5, 6, 7, 8, 9, 10 or more amino acid residues selected from the 45th amino acid residue to the 100th amino acid residue of the amino acid sequence of IL-36α represented by SEQ ID NO: 5 or SEQ ID NO: 7 and/or the amino acid sequence of IL-36γ represented by SEQ ID NO: 10. In some embodiments, the antibody or fragment thereof provided herein binds to more than 10, 15, 20, 25, or 30 amino acid residues selected from the 45th amino acid residue to the 100th amino acid residue of the amino acid sequence of IL-36α represented by SEQ ID NO: 5 or SEQ ID NO: 7 and/or the amino acid sequence of IL-36γ represented by SEQ ID NO: 10.

In some embodiments, the antibody or antigen binding fragment thereof binds to one or more amino acid residues selected from Arg 45, His 46, Glu 48, Thr 49, Leu 50, Lys 85, Asp 89, Asn 92, Gln 93, Pro 94, Glu 95, Pro 96, Val 97, Lys 98 and Phe 100 of the amino acid sequence of IL-36α represented by SEQ ID NO: 5 or SEQ ID NO: 7. In some embodiments, the antibody or antigen binding fragment thereof binds to 2, 3, 4, 5, 6, 7, 8, 9, 10, 11, 12, 13, 14 or 15 amino acid residues selected from Arg 45, His 46, Glu 48, Thr 49, Leu 50, Lys 85, Asp 89, Asn 92, Gln 93, Pro 94, Glu 95, Pro 96, Val 97, Lys 98 and Phe 100 of the amino acid sequence of IL-36α represented by SEQ ID NO: 5 or SEQ ID NO: 7.

In other embodiments, the antibody or antigen binding fragment thereof binds to one or more amino acid residues selected from one Tyr 46, Glu 48, Ala 49, Leu 50, Gln 85, Gly 92, Gln 93, Pro 94, Glu 95, Pro 96, Val 97, Lys 98 and Phe 100 of the amino acid sequence of IL-36γ represented by SEQ ID NO: 10. In other embodiments, the antibody or antigen binding fragment thereof binds to 2, 3, 4, 5, 6, 7, 8, 9, 10, 11, 12, or 13 amino acid residues selected from one Tyr 46, Glu 48, Ala 49, Leu 50, Gln 85, Gly 92, Gln 93, Pro 94, Glu 95, Pro 96, Val 97, Lys 98 and Phe 100 of the amino acid sequence of IL-36γ represented by SEQ ID NO: 10.

In other embodiments, the antibody or antigen binding fragment thereof binds to one or more amino acid residues selected from Arg 45, His 46, Glu 48, Thr 49, Leu 50, Lys 85, Asp 89, Asn 92, Gln 93, Pro 94, Glu 95, Pro 96, Val 97, Lys 98 and Phe 100 of the amino acid sequence of IL-36α represented by SEQ ID NO: 5 or SEQ ID NO: 7; and to one or more amino acid residues selected from one Tyr 46, Glu 48, Ala 49, Leu 50, Gln 85, Gly 92, Gln 93, Pro 94, Glu 95, Pro 96, Val 97, Lys 98 and Phe 100 of the amino acid sequence of IL-36γ represented by SEQ ID NO: 10.

In some embodiments, the antibody or antigen binding fragment thereof binds to one or more amino acid residues selected from His 46, Glu 48, Thr 49, Leu 50, Lys 85, Gln 93, Pro 94, Glu 95, Pro 96, Val 97 and Lys 98 of the amino acid sequence of IL-36α represented by SEQ ID NO: 5 or SEQ ID NO: 7. In some embodiments, the antibody or antigen binding fragment thereof binds to 2, 3, 4, 5, 6, 7, 8, 9, 10, or 11 amino acid residues selected from His 46, Glu 48, Thr 49, Leu 50, Lys 85, Gln 93, Pro 94, Glu 95, Pro 96, Val 97 and Lys 98 of the amino acid sequence of IL-36α represented by SEQ ID NO: 5 or SEQ ID NO: 7.

In some embodiments, the antibody or antigen binding fragment thereof binds to one or more amino acid residues selected from Ala 49, Leu 50, Gly 92, Gln 93, Pro 94, Glu 95, Pro 96, Val 97 and Lys 98 of the amino acid sequence of IL-36γ represented by SEQ ID NO: 10. In some embodiments, the antibody or antigen binding fragment thereof binds to 2, 3, 4, 5, 6, 7, 8, or 9 amino acid residues selected from Ala 49, Leu 50, Gly 92, Gln 93, Pro 94, Glu 95, Pro 96, Val 97 and Lys 98 of the amino acid sequence of IL-36γ represented by SEQ ID NO: 10.

In other embodiments, the antibody or antigen binding fragment thereof binds to one or more amino acid residues selected from His 46, Glu 48, Thr 49, Leu 50, Lys 85, Gln 93, Pro 94, Glu 95, Pro 96, Val 97 and Lys 98 of the amino acid sequence of IL-36α represented by SEQ ID NO: 5 or SEQ ID NO: 7, and to one or more amino acid residues selected from Ala 49, Leu 50, Gly 92, Gln 93, Pro 94, Glu 95, Pro 96, Val 97 and Lys 98 of the amino acid sequence of IL-36γ represented by SEQ ID NO: 10.

In some more specific embodiments, the antibody or antigen binding fragment thereof binds to at least one of amino acid residues selected from Leu 50, Gln 93, Pro 94, Glu 95, Pro 96, Val 97 and Lys 98 of both the amino acid sequence of IL-36α represented by SEQ ID NO: 5 or SEQ ID NO: 7 and the amino acid sequence of IL-36γ represented by SEQ ID NO: 10. In some embodiments, the antibody or antigen binding fragment thereof binds to 2, 3, 4, 5, 6 or 7 amino acid residues selected from Leu 50, Gln 93, Pro 94, Glu 95, Pro 96, Val 97 and Lys 98 of both the amino acid sequence of IL-36α represented by SEQ ID NO: 5 or SEQ ID NO: 7 and the amino acid sequence of IL-36γ represented by SEQ ID NO: 10.

In a specific embodiments, the antibody or antigen binding fragment thereof binds to the 93rd to 98th amino acid residues of the amino acid sequence of IL-36α represented by SEQ ID NO: 5 or SEQ ID NO: 7 and the amino acid sequence of IL-36γ represented by SEQ ID NO: 10. In another specific embodiments, the antibody or antigen binding fragment thereof binds to the 93rd to 97th amino acid residues of the amino acid sequence of IL-36α represented by SEQ ID NO: 5 or SEQ ID NO: 7 and the amino acid sequence of IL-36γ represented by SEQ ID NO: 10. In another specific embodiment, the antibody or antigen binding fragment thereof binds to the 50th and 93rd to 98th amino acid residues of the amino acid sequence of IL-36α represented by SEQ ID NO: 5 or SEQ ID NO: 7 and the amino acid sequence of IL-36γ represented by SEQ ID NO: 10. In yet another specific embodiment, the antibody or antigen binding fragment thereof binds to the 50th and 93rd to 97th amino acid residues of the amino acid sequence of IL-36α represented by SEQ ID NO: 5 or SEQ ID NO: 7 and the amino acid sequence of IL-36γ represented by SEQ ID NO: 10.

In the specific embodiments described in the paragraph above, the antibody or antigen binding fragment thereof may further bind to one or more amino acid residues in SEQ ID NO: 5, SEQ ID NO: 7 or SEQ ID NO: 10. In some embodiments, the antibody or antigen binding fragment thereof further binds to 1, 2, 3, 4, 5, 6, 7, 8, 9, 10, or more amino acid residues in SEQ ID NO: 5, SEQ ID NO: 7 or SEQ ID NO: 10. For example, in some embodiments, the antibody or antigen binding fragment thereof further binds to at least one of amino acid residue selected from Arg 45, His 46, Glu 48, Thr 49, Lys 85, Asp 89, Asn 92 and Phe 100 of the amino acid sequence of IL-36α represented by SEQ ID NO: 5 or SEQ ID NO: 7, and/or at least one of amino acid residue selected from Tyr 46, Glu 48, Ala 49, Gln 85, Gly 92 and Phe 100 of the amino acid sequence of IL-36γ represented by SEQ ID NO: 10. In other embodiments, the antibody or antigen binding fragment thereof further binds to at least one of amino acid residues selected from His 46, Glu 48, Thr 49 and Lys 85 amino acid sequence of IL-36α represented by SEQ ID NO: 5 or SEQ ID NO: 7, and at least one of IL-36γ amino acid residues selected from Ala 49 and Gly 92 of the amino acid sequence of IL-36γ represented by SEQ ID NO: 10.

In some embodiments, the antibody or antigen binding fragment provided herein binds to IL-36α with a K_{D} of less than 1000 nM. In some embodiments, the antibody or antigen binding fragment provided herein binds to IL-36α with a K_{D} of less than 100 nM. In some embodiments, the antibody or antigen binding fragment provided herein binds to IL-36α with a K_{D} of less than 50 nM. In some embodiments, the antibody or antigen binding fragment provided herein binds to IL-36α with a K_{D} of less than 40 nM. In some embodiments, the antibody or antigen binding fragment provided herein binds to IL-36α with a K_{D} of less than 30 nM. In some embodiments, the antibody or antigen binding fragment provided herein binds to IL-36α with a K_{D} of less than 20 nM. In some embodiments, the antibody or antigen binding fragment provided herein binds to IL-36α with a K_{D} of less than 10 nM. In some embodiments, the antibody or antigen binding fragment provided herein binds to IL-36α with a K_{D} of less than 9 nM. In some embodiments, the antibody or antigen binding fragment provided herein binds to IL-36α with a K_{D} of less than 8 nM. In some embodiments, the antibody or antigen binding fragment provided herein binds to IL-36α with a K_{D} of less than 7 nM. In some embodiments, the antibody or antigen binding fragment provided herein binds to IL-36α with a K_{D} of less than 6 nM. In some embodiments, the antibody or antigen binding fragment provided herein binds to IL-36α with a K_{D} of less than 5 nM. In some embodiments, the antibody or antigen binding fragment provided herein binds to IL-36α with a K_{D} of less than 4 nM. In some embodiments, the antibody or antigen binding fragment provided herein binds to IL-36α with a K_{D} of less than 3 nM. In some embodiments, the antibody or antigen binding fragment provided herein binds to IL-36α with a K_{D} of less than 2 nM. In some embodiments, the antibody or antigen binding fragment provided herein binds to IL-36α with a K_{D} of less than 1 nM. In some embodiments, the antibody or antigen binding fragment provided herein binds to IL-36α with a K_{D} of less than 0.1 nM. In some embodiments, the antibody or antigen binding fragment provided herein binds to IL-36α with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®™-2000 or a Biacore®™-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®™-2000, or the Biacore®™-3000 system. In a specific embodiment, the K_{D} is determined by a Biacore® assay. In some embodiments, IL-36α is a human IL-36a. In some embodiments, IL-36α is a cynomolgus macaque IL-36α.

In some embodiments, the antibody or antigen binding fragment provided herein binds to IL-36γ with a K_{D} of less than 1000 nM. In some embodiments, the antibody or antigen binding fragment provided herein binds to IL-36γ with a K_{D} of less than 100 nM. In some embodiments, the antibody or antigen binding fragment provided herein binds to IL-36γ with a K_{D} of less than 50 nM. In some embodiments, the antibody or antigen binding fragment provided herein binds to IL-36γ with a K_{D} of less than 40 nM. In some embodiments, the antibody or antigen binding fragment provided herein binds to IL-36γ with a K_{D} of less than 30 nM. In some embodiments, the antibody or antigen binding fragment provided herein binds to IL-36γ with a K_{D} of less than 20 nM. In some embodiments, the antibody or antigen binding fragment provided herein binds to IL-36γ with a K_{D} of less than 10 nM. In some embodiments, the antibody or antigen binding fragment provided herein binds to IL-36γ with a K_{D} of less than 9 nM. In some embodiments, the antibody or antigen binding fragment provided herein binds to IL-36γ with a K_{D} of less than 8 nM. In some embodiments, the antibody or antigen binding fragment provided herein binds to IL-36γ with a K_{D} of less than 7 nM. In some embodiments, the antibody or antigen binding fragment provided herein binds to IL-36γ with a K_{D} of less than 6 nM. In some embodiments, the antibody or antigen binding fragment provided herein binds to IL-36γ with a K_{D} of less than 5 nM. In some embodiments, the antibody or antigen binding fragment provided herein binds to IL-36γ with a K_{D} of less than 4 nM. In some embodiments, the antibody or antigen binding fragment provided herein binds to IL-36γ with a K_{D} of less than 3 nM. In some embodiments, the antibody or antigen binding fragment provided herein binds to IL-36γ with a K_{D} of less than 2 nM. In some embodiments, the antibody or antigen binding fragment provided herein binds to IL-36γ with a K_{D} of less than 1 nM. In some embodiments, the antibody or antigen binding fragment provided herein binds to IL-36γ with a K_{D} of less than 0.1 nM. In some embodiments, the antibody or antigen binding fragment provided herein binds to IL-36γ with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®™-2000 or a Biacore®™-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®™-2000, or the Biacore®™-3000 system. In a specific embodiment, the K_{D} is determined by a Biacore® assay. In some embodiments, IL-36γ is a human IL-36γ. In some embodiments, IL-36γ is a cynomolgus macaque IL-36γ.

In certain embodiments, the antibody or antigen binding fragment provided herein binds to both IL-36α and IL-36γ. In some embodiments, the antibody or antigen binding fragment thereof provided herein binds to IL-36α with a K_{D} of less than 1000 nM as determined by a Biacore® assay, and binds to IL-36γ with a K_{D} of less than 1000 nM as determined by a Biacore® assay. In some embodiments, the antibody or antigen binding fragment provided herein binds to IL-36α with a K_{D} of less than 100 nM as determined by a Biacore® assay, and binds to IL-36γ with a K_{D} of less than 100 nM as determined by a Biacore® assay. In some embodiments, the antibody or antigen binding fragment provided herein binds to IL-36α with a K_{D} of less than 90 nM as determined by a Biacore® assay, and binds to IL-36γ with a K_{D} of less than 90 nM as determined by a Biacore® assay. In some embodiments, the antibody or antigen binding fragment provided herein binds to IL-36α with a K_{D} of less than 80 nM as determined by a Biacore® assay, and binds to IL-36γ with a K_{D} of less than 80 nM as determined by a Biacore® assay. In some embodiments, the antibody or antigen binding fragment provided herein binds to IL-36α with a K_{D} of less than 70 nM as determined by a Biacore® assay, and binds to IL-36γ with a K_{D} of less than 70 nM as determined by a Biacore® assay. In some embodiments, the antibody or antigen binding fragment provided herein binds to IL-36α with a KD of less than 60 nM as determined by a Biacore® assay, and binds to IL-36γ with a K_{D} of less than 60 nM as determined by a Biacore® assay. In some embodiments, the antibody or antigen binding fragment provided herein binds to IL-36α with a K_{D} of less than 50 nM as determined by a Biacore® assay, and binds to IL-36γ with a K_{D} of less than 50 nM as determined by a Biacore® assay. In some embodiments, the antibody or antigen binding fragment provided herein binds to IL-36α with a K_{D} of less than 40 nM as determined by a Biacore® assay, and binds to IL-36γ with a K_{D} of less than 40 nM as determined by a Biacore® assay. In some embodiments, the antibody or antigen binding fragment provided herein binds to IL-36α with a K_{D} of less than 30 nM as determined by a Biacore® assay, and binds to IL-36γ with a K_{D} of less than 30 nM as determined by a Biacore® assay. In some embodiments, the antibody or antigen binding fragment provided herein binds to IL-36α with a K_{D} of less than 20 nM as determined by a Biacore® assay, and binds to IL-36γ with a K_{D} of less than 20 nM as determined by a Biacore® assay. In some embodiments, the antibody or antigen binding fragment provided herein binds to IL-36α with a K_{D} of less than 10 nM as determined by a Biacore® assay, and binds to IL-36γ with a K_{D} of less than 10 nM as determined by a Biacore® assay. In some embodiments, the antibody or antigen binding fragment provided herein binds to IL-36α with a K_{D} of less than 1nM as determined by a Biacore® assay, and binds to IL-36γ with a K_{D} of less than 1 nM as determined by a Biacore® assay. In some embodiments, the antibody or antigen binding fragment provided herein binds to IL-36α with a K_{D} of less than 0.1 nM as determined by a Biacore® assay, and binds to IL-36γ with a K_{D} of less than 0.1 nM as determined by a Biacore® assay. In some embodiments, the antibody or antigen binding fragment provided herein binds to IL-36α with a K_{D} of less than 0.01 nM as determined by a Biacore® assay, and binds to IL-36γ with a K_{D} of less than 0.01 nM as determined by a Biacore® assay. In some embodiments, IL-36α is a human IL-36α. In some embodiments, IL-36α is a cynomolgus macaque IL-36α. In some embodiments, IL-36γ is a human IL-36γ. In some embodiments, IL-36γ is a cynomolgus macaque IL-36γ.

In some embodiments, the antibody or antigen binding fragment thereof provided herein binds to IL-36α with a K_{D} of less than 100 nM as determined by a Biacore® assay, and binds to IL-36γ with a K_{D} of less than 90 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a KD of less than 80 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 70 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 60 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 50 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 40 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 30 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 20 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 10 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 1 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 0.1 nM as determined by a Biacore® assay. In some embodiments, the antibody or antigen binding fragment thereof provided herein binds to IL-36α with a K_{D} of less than 100 nM as determined by a Biacore® assay, and binds to IL-36γ with a K_{D} of less than 0.01nM as determined by a Biacore® assay. In some embodiments, IL-36α is a human IL-36a. In some embodiments, IL-36α is a cynomolgus macaque IL-36α. In some embodiments, IL-36γ is a human IL-36γ. In some embodiments, IL-36γ is a cynomolgus macaque IL-36γ.

In some embodiments, the antibody or antigen binding fragment thereof provided herein binds to IL-36γ with a K_{D} of less than 100 nM as determined by a Biacore® assay, and binds to IL-36α with a K_{D} of less than 90 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 80 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 70 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 60 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 50 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 40 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 30 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 20 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 10 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 1 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 0.1 nM as determined by a Biacore® assay. In some embodiments, the antibody or antigen binding fragment thereof provided herein binds to IL-36γ with a K_{D} of less than 100 nM as determined by a Biacore® assay, and binds to IL-36α with a K_{D} of less than 0.01nM as determined by a Biacore® assay. In some embodiments, IL-36α is a human IL-36α. In some embodiments, IL-36α is a cynomolgus macaque IL-36α. In some embodiments, IL-36γ is a human IL-36γ. In some embodiments, IL-36γ is a cynomolgus macaque IL-36γ.

In one aspect, provided herein are antibodies that specifically bind to IL-36α and can modulate IL-36α activity and/or expression (e.g., inhibit IL-36α mediated signaling). In certain embodiments, an IL-36α antagonist is provided herein that is an antibody described herein that specifically binds to IL-36α and inhibits (including partially inhibits) at least one IL-36α activity. In some embodiments, the antibodies provided herein inhibit (including partially inhibit or reduce) the binding of IL-36α to its receptor.

An IL-36α activity can relate to any activity of IL-36α such as those known or described in the art. In certain embodiments, IL-36α activity and IL-36α signaling (or IL-36a mediated signaling) are used interchangeably herein. In certain aspects, IL-36α activity is induced by IL-36 receptor (e.g., IL-36α binding to IL-36 receptor). In certain embodiments, provided herein are antibodies that specifically bind to IL-36α and inhibit (or reduce) cytokine production. In some embodiments, the antibodies provided herein do not inhibit the binding of IL-36α to IL-36 receptor, but nevertheless inhibit or reduce the IL-36α mediated or IL-36 receptor mediated signaling.

In certain embodiments, the antibody described herein attenuates (e.g., partially attenuates) an IL-36α activity. In some embodiments, the antibody provided herein attenuates an IL-36α activity by at least about 10%. In some embodiments, the antibody provided herein attenuates an IL-36α activity by at least about 20%. In some embodiments, the antibody provided herein attenuates an IL-36α activity by at least about 30%. In some embodiments, the antibody provided herein attenuates an IL-36α activity by at least about 40%. In some embodiments, the antibody provided herein attenuates an IL-36α activity by at least about 50%. In some embodiments, the antibody provided herein attenuates an IL-36α activity by at least about 60%. In some embodiments, the antibody provided herein attenuates an IL-36α activity by at least about 70%. In some embodiments, the antibody provided herein attenuates an IL-36α activity by at least about 80%. In some embodiments, the antibody provided herein attenuates an IL-36α activity by at least about 90%. In some embodiments, the antibody provided herein attenuates an IL-36α activity by at least about 95%. In certain embodiments, the antibody described herein can attenuate (e.g., partially attenuate) an IL-36α activity by at least about 15% to about 65%. In certain embodiments, the antibody described herein can attenuate (e.g., partially attenuate) an IL-36α activity by at least about 20% to about 65%. In certain embodiments, the antibody described herein can attenuate (e.g., partially attenuate) an IL-36α activity by at least about 30% to about 65%.

In specific embodiments, the attenuation of an IL-36α activity is assessed by methods described herein. In specific embodiments, the attenuation of an IL-36α activity is assessed by methods known to one of skill in the art. In certain embodiments, the attenuation of an IL-36α activity is relative to the IL-36α activity in the presence of stimulation without any anti-IL-36α antibody. In certain embodiments, the attenuation of an IL-36α activity is relative to the IL-36α activity in the presence of stimulation with an unrelated antibody (e.g., an antibody that does not specifically bind to IL-36α).

A non-limiting example of an IL-36α activity is IL-36α mediated signaling. Thus, in certain embodiments, the antibody described herein attenuates (e.g., partially attenuates) IL-36α mediated signaling. In some embodiments, the antibody provided herein attenuates IL-36α mediated signaling by at least about 10%. In some embodiments, the antibody provided herein attenuates IL-36α mediated signaling by at least about 20%. In some embodiments, the antibody provided herein attenuates IL-36α mediated signaling by at least about 30%. In some embodiments, the antibody provided herein attenuates IL-36α mediated signaling by at least about 40%. In some embodiments, the antibody provided herein attenuates IL-36α mediated signaling by at least about 50%. In some embodiments, the antibody provided herein attenuates IL-36α mediated signaling by at least about 60%. In some embodiments, the antibody provided herein attenuates IL-36α mediated signaling by at least about 70%. In some embodiments, the antibody provided herein attenuates IL-36α mediated signaling by at least about 80%. In some embodiments, the antibody provided herein attenuates IL-36α mediated signaling by at least about 90%. In some embodiments, the antibody provided herein attenuates IL-36α mediated signaling by at least about 95%. In certain embodiments, the antibody described herein can attenuate (e.g., partially attenuate) IL-36α mediated signaling by at least about 15% to about 65%. In certain embodiments, the antibody described herein can attenuate (e.g., partially attenuate) IL-36α mediated signaling by at least about 20% to about 65%. In certain embodiments, the antibody described herein can attenuate (e.g., partially attenuate) IL-36α mediated signaling by at least about 30% to about 65%.

Another non-limiting example of an IL-36α activity is binding to IL-36 receptor. Thus, in certain embodiments, the antibody described herein attenuates (e.g., partially attenuates) the binding of IL-36α to an IL-36 receptor. In some embodiments, the antibody provided herein attenuates the binding of IL-36α to an IL-36 receptor by at least about 10%. In some embodiments, the antibody provided herein attenuates the binding of IL-36α to an IL-36 receptor by at least about 20%. In some embodiments, the antibody provided herein attenuates the binding of IL-36α to an IL-36 receptor by at least about 30%. In some embodiments, the antibody provided herein attenuates the binding of IL-36α to an IL-36 receptor by at least about 40%. In some embodiments, the antibody provided herein attenuates the binding of IL-36α to an IL-36 receptor by at least about 50%. In some embodiments, the antibody provided herein attenuates the binding of IL-36α to an IL-36 receptor by at least about 60%. In some embodiments, the antibody provided herein attenuates the binding of IL-36α to an IL-36 receptor by at least about 70%. In some embodiments, the antibody provided herein attenuates the binding of IL-36α to an IL-36 receptor by at least about 80%. In some embodiments, the antibody provided herein attenuates the binding of IL-36α to an IL-36 receptor by at least about 90%. In some embodiments, the antibody provided herein attenuates the binding of IL-36α to an IL-36 receptor by at least about 95%. In certain embodiments, the antibody described herein can attenuate (e.g., partially attenuate) the binding of IL-36α to an IL-36 receptor by at least about 15% to about 65%. In certain embodiments, the antibody described herein can attenuate (e.g., partially attenuate) the binding of IL-36α to an IL-36 receptor by at least about 20% to about 65%. In certain embodiments, the antibody described herein can attenuate (e.g., partially attenuate) the binding of IL-36α to an IL-36 receptor by at least about 30% to about 65%.

Another non-limiting example of an IL-36α activity is signaling mediated by an IL-36 receptor. Thus, in certain embodiments, the antibody described herein attenuates (e.g., partially attenuates) IL-36 receptor mediated signaling. In some embodiments, the antibody provided herein attenuates IL-36 receptor mediated signaling by at least about 10%. In some embodiments, the antibody provided herein attenuates IL-36 receptor mediated signaling by at least about 20%. In some embodiments, the antibody provided herein attenuates IL-36 receptor mediated signaling by at least about 30%. In some embodiments, the antibody provided herein attenuates IL-36 receptor mediated signaling by at least about 40%. In some embodiments, the antibody provided herein attenuates IL-36 receptor mediated signaling by at least about 50%. In some embodiments, the antibody provided herein attenuates IL-36 receptor mediated signaling by at least about 60%. In some embodiments, the antibody provided herein attenuates IL-36 receptor mediated signaling by at least about 70%. In some embodiments, the antibody provided herein attenuates IL-36 receptor mediated signaling by at least about 80%. In some embodiments, the antibody provided herein attenuates IL-36 receptor mediated signaling by at least about 90%. In some embodiments, the antibody provided herein attenuates IL-36 receptor mediated signaling by at least about 95%. In certain embodiments, the antibody described herein can attenuate (e.g., partially attenuate) IL-36 receptor mediated signaling by at least about 15% to about 65%. In certain embodiments, the antibody described herein can attenuate (e.g., partially attenuate) IL-36 receptor mediated signaling by at least about 20% to about 65%. In certain embodiments, the antibody described herein can attenuate (e.g., partially attenuate) IL-36 receptor mediated signaling by at least about 30% to about 65%.

In specific embodiments, antibodies provided herein (e.g., any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c, or an antigen-binding fragment thereof, or an antibody comprising CDRs of any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c) specifically bind to IL-36α and inhibit the secretion of one or more cytokines and/or chemokines induced by IL-36α. In some embodiments, the one or more cytokines and/or chemokines are selected from a group consisting of IL-8, IL-6, IL-10, TNFα, IL-1β, CXCL1, CCLS, CCL20, CCL2, CCL3, CCL4, CXCL12, VEGF-A, IL-23, IL-36α, IL-36β, and IL-36γ.

For example, in one embodiment, an antibody provided herein specifically binds to IL-36α and inhibits IL-8 secretion by at least about 5%. In one embodiment, an antibody provided herein specifically binds to IL-36α and inhibits IL-8 secretion by at least about 10%. In one embodiment, an antibody provided herein specifically binds to IL-36α and inhibits IL-8 secretion by at least about 15%. In one embodiment, an antibody provided herein specifically binds to IL-36α and inhibits IL-8 secretion by at least about 20%. In one embodiment, an antibody provided herein specifically binds to IL-36α and inhibits IL-8 secretion by at least about 25%. In one embodiment, an antibody provided herein specifically binds to IL-36α and inhibits IL-8 secretion by at least about 30%. In one embodiment, an antibody provided herein specifically binds to IL-36α and inhibits IL-8 secretion by at least about 35%. In one embodiment, an antibody provided herein specifically binds to IL-36α and inhibits IL-8 secretion by at least about 40%. In one embodiment, an antibody provided herein specifically binds to IL-36α and inhibits IL-8 secretion by at least about 45%. In one embodiment, an antibody provided herein specifically binds to IL-36α and inhibits IL-8 secretion by at least about 50%. In one embodiment, an antibody provided herein specifically binds to IL-36α and inhibits IL-8 secretion by at least about 55%. In one embodiment, an antibody provided herein specifically binds to IL-36α and inhibits IL-8 secretion by at least about 60%. In one embodiment, an antibody provided herein specifically binds to IL-36α and inhibits IL-8 secretion by at least about 65%. In one embodiment, an antibody provided herein specifically binds to IL-36α and inhibits IL-8 secretion by at least about 70%. In one embodiment, an antibody provided herein specifically binds to IL-36α and inhibits IL-8 secretion by at least about 75%. In one embodiment, an antibody provided herein specifically binds to IL-36α and inhibits IL-8 secretion by at least about 80%. In one embodiment, an antibody provided herein specifically binds to IL-36α and inhibits IL-8 secretion by at least about 85%. In one embodiment, an antibody provided herein specifically binds to IL-36α and inhibits IL-8 secretion by at least about 90%. In one embodiment, an antibody provided herein specifically binds to IL-36α and inhibits IL-8 secretion by at least about 95%. In one embodiment, an antibody provided herein specifically binds to IL-36α and inhibits IL-8 secretion by at least about 96%. In one embodiment, an antibody provided herein specifically binds to IL-36α and inhibits IL-8 secretion by at least about 97%. In one embodiment, an antibody provided herein specifically binds to IL-36α and inhibits IL-8 secretion by at least about 98%. In one embodiment, an antibody provided herein specifically binds to IL-36α and inhibits IL-8 secretion by at least about 99%. In some embodiments, the inhibition of IL-8 secretion is assessed by methods described herein. In other embodiments, the inhibition of IL-8 secretion is assessed by methods known to one of skill in the art. In a specific embodiment, the IL-8 secretion is inhibited relative to IL-8 secretion in the absence of anti-IL-36α antibody. In other embodiments, the IL-8 secretion is inhibited relative to IL-8 secretion in the presence of an unrelated antibody (e.g., an antibody that does not specifically bind to IL-36α).

In one embodiment, the antibody provided herein inhibits IL-8 secretion with an IC₅₀ of at most about 100 nM. In one embodiment, the antibody provided herein inhibits IL-8 secretion with an IC₅₀ of at most about 90 nM. In one embodiment, the antibody provided herein inhibits IL-8 secretion with an IC₅₀ of at most about 80 nM. In one embodiment, the antibody provided herein inhibits IL-8 secretion with an IC₅₀ of at most about 70 nM. In one embodiment, the antibody provided herein inhibits IL-8 secretion with an IC₅₀ of at most about 60 nM. In one embodiment, the antibody provided herein inhibits IL-8 secretion with an IC₅₀ of at most about 50 nM. In one embodiment, the antibody provided herein inhibits IL-8 secretion with an IC₅₀ of at most about 40 nM. In one embodiment, the antibody provided herein inhibits IL-8 secretion with an IC₅₀ of at most about 30 nM. In one embodiment, the antibody provided herein inhibits IL-8 secretion with an IC₅₀ of at most about 20 nM. In one embodiment, the antibody provided herein inhibits IL-8 secretion with an IC₅₀ of at most about 10 nM. In one embodiment, the antibody provided herein inhibits IL-8 secretion with an IC₅₀ of at most about 1 nM. In one embodiment, the antibody provided herein inhibits IL-8 secretion with an IC₅₀ of at most about 0.1 nM. In one embodiment, the antibody provided herein inhibits IL-8 secretion with an IC₅₀ of at most about 0.05 nM. In one embodiment, the antibody provided herein inhibits IL-8 secretion with an IC₅₀ of at most about 0.001 nM.

In one embodiment, the antibody provided herein inhibits IL-8 secretion with an IC₅₀ of at least about 100 nM. In one embodiment, the antibody provided herein inhibits IL-8 secretion with an IC₅₀ of at least about 90 nM. In one embodiment, the antibody provided herein inhibits IL-8 secretion with an IC₅₀ of at least about 80 nM. In one embodiment, the antibody provided herein inhibits IL-8 secretion with an IC₅₀ of at least about 70 nM. In one embodiment, the antibody provided herein inhibits IL-8 secretion with an IC₅₀ of at least about 60 nM. In one embodiment, the antibody provided herein inhibits IL-8 secretion with an IC₅₀ of at least about 50 nM. In one embodiment, the antibody provided herein inhibits IL-8 secretion with an IC₅₀ of at least about 40 nM. In one embodiment, the antibody provided herein inhibits IL-8 secretion with an IC₅₀ of at least about 30 nM. In one embodiment, the antibody provided herein inhibits IL-8 secretion with an IC₅₀ of at least about 20 nM. In one embodiment, the antibody provided herein inhibits IL-8 secretion with an IC₅₀ of at least about 10 nM. In one embodiment, the antibody provided herein inhibits IL-8 secretion with an IC₅₀ of at least about 1 nM. In one embodiment, the antibody provided herein inhibits IL-8 secretion with an IC₅₀ of at least about 0.1 nM. In one embodiment, the antibody provided herein inhibits IL-8 secretion with an IC₅₀ of at least about 0.05 nM. In one embodiment, the antibody provided herein inhibits IL-8 secretion with an IC50 of at least about 0.001 nM. In specific embodiments, the IC₅₀ is assessed by methods described herein, for example, in Section 6 below. In other embodiments, the IC₅₀ is assessed by other methods known to one of skill in the art.

In certain embodiments, the antibody provided herein binds to IL-36α and attenuates (e.g., partially attenuates) IL-36 receptor dimerization (i.e., heterodimerization between IL-36R (also known as IL-1Rrp2) and IL-1RAcP (also known as IL-1 receptor accessory protein)). In some embodiments, the antibody provided herein binds to IL-36α and attenuates IL-36 receptor dimerization by at least about 10%. In some embodiments, the antibody provided herein binds to IL-36α and attenuates IL-36 receptor dimerization by at least about 15%. In some embodiments, the antibody provided herein binds to IL-36α and attenuates IL-36 receptor dimerization by at least about 20%. In some embodiments, the antibody provided herein binds to IL-36α and attenuates IL-36 receptor dimerization by at least about 25%. In some embodiments, the antibody provided herein binds to IL-36α and attenuates IL-36 receptor dimerization by at least about 30%. In some embodiments, the antibody provided herein binds to IL-36α and attenuates IL-36 receptor dimerization by at least about 35%. In some embodiments, the antibody provided herein binds to IL-36α and attenuates IL-36 receptor dimerization by at least about 40%. In some embodiments, the antibody provided herein binds to IL-36α and attenuates IL-36 receptor dimerization by at least about 45%. In some embodiments, the antibody provided herein binds to IL-36α and attenuates IL-36 receptor dimerization by at least about 50%. In some embodiments, the antibody provided herein binds to IL-36α and attenuates IL-36 receptor dimerization by at least about 55%. In some embodiments, the antibody provided herein binds to IL-36α and attenuates IL-36 receptor dimerization by at least about 60%. In some embodiments, the antibody provided herein binds to IL-36α and attenuates IL-36 receptor dimerization by at least about 65%. In some embodiments, the antibody provided herein binds to IL-36α and attenuates IL-36 receptor dimerization by at least about 70%. In some embodiments, the antibody provided herein binds to IL-36α and attenuates IL-36 receptor dimerization by at least about 75%. In some embodiments, the antibody provided herein binds to IL-36α and attenuates IL-36 receptor dimerization by at least about 80%. In some embodiments, the antibody provided herein binds to IL-36α and attenuates IL-36 receptor dimerization by at least about 85%. In some embodiments, the antibody provided herein binds to IL-36α and attenuates IL-36 receptor dimerization by at least about 90%. In some embodiments, the antibody provided herein binds to IL-36α and attenuates IL-36 receptor dimerization by at least about 95%.

In certain embodiments, the antibody provided herein binds to IL-36α and attenuates (e.g., partially attenuates) activation of mitogen-activated protein kinase (MAPK) pathways and/or nuclear factor kappa B (NF-κB) dependent transcription. In certain embodiments, the antibody provided herein binds to IL-36α and attenuates activation of MAPK pathways and/or NF-κB dependent transcription by at least about 10%. In certain embodiments, the antibody provided herein binds to IL-36α and attenuates activation of MAPK pathways and/or NF-κB dependent transcription by at least about 15%. In certain embodiments, the antibody provided herein binds to IL-36α and attenuates activation of MAPK pathways and/or NF-κB dependent transcription by at least about 20%. In certain embodiments, the antibody provided herein binds to IL-36α and attenuates activation of MAPK pathways and/or NF-κB dependent transcription by at least about 25%. In certain embodiments, the antibody provided herein binds to IL-36α and attenuates activation of MAPK pathways and/or NF-κB dependent transcription by at least about 30%. In certain embodiments, the antibody provided herein binds to IL-36α and attenuates activation of MAPK pathways and/or NF-κB dependent transcription by at least about 35%. In certain embodiments, the antibody provided herein binds to IL-36α and attenuates activation of MAPK pathways and/or NF-κB dependent transcription by at least about 40%. In certain embodiments, the antibody provided herein binds to IL-36α and attenuates activation of MAPK pathways and/or NF-κB dependent transcription by at least about 45%. In certain embodiments, the antibody provided herein binds to IL-36α and attenuates activation of MAPK pathways and/or NF-κB dependent transcription by at least about 50%. In certain embodiments, the antibody provided herein binds to IL-36α and attenuates activation of MAPK pathways and/or NF-κB dependent transcription by at least about 60%. In certain embodiments, the antibody provided herein binds to IL-36α and attenuates activation of MAPK pathways and/or NF-κB dependent transcription by at least about 65%. In certain embodiments, the antibody provided herein binds to IL-36α and attenuates activation of MAPK pathways and/or NF-κB dependent transcription by at least about 70%. In certain embodiments, the antibody provided herein binds to IL-36α and attenuates activation of MAPK pathways and/or NF-κB dependent transcription by at least about 75%. In certain embodiments, the antibody provided herein binds to IL-36α and attenuates activation of MAPK pathways and/or NF-κB dependent transcription by at least about 80%. In certain embodiments, the antibody provided herein binds to IL-36α and attenuates activation of MAPK pathways and/or NF-κB dependent transcription by at least about 85%. In certain embodiments, the antibody provided herein binds to IL-36α and attenuates activation of MAPK pathways and/or NF-κB dependent transcription by at least about 90%. In certain embodiments, the antibody provided herein binds to IL-36α and attenuates activation of MAPK pathways and/or NF-κB dependent transcription by at least about 95%.

In another aspect, provided herein are antibodies that specifically bind to IL-36γ and can modulate IL-36γ activity and/or expression (e.g., inhibit IL-36γ mediated signaling). In certain embodiments, an IL-36γ antagonist is provided herein that is an antibody described herein that specifically binds to IL-36γ and inhibits (including partially inhibits) at least one IL-36γ activity. In some embodiments, the antibodies provided herein inhibit (including partially inhibit or reduce) the binding of IL-36γ to its receptor.

An IL-36γ activity can relate to any activity of IL-36γ such as those known or described in the art. In certain embodiments, IL-36γ activity and IL-36γ signaling (or IL-36γ mediated signaling) are used interchangeably herein. In certain aspects, IL-36γ activity is induced by IL-36 receptor (e.g., IL-36γ binding to IL-36 receptor). In certain embodiments, provided herein are antibodies that specifically bind to IL-36γ and inhibit (or reduce) cytokine production. In some embodiments, the antibodies provided herein do not inhibit the binding of IL-36γ to IL-36 receptor, but nevertheless inhibit or reduce the IL-36γ mediated or IL-36 receptor mediated signaling.

In certain embodiments, the antibody described herein attenuates (e.g., partially attenuates) an IL-36γ activity. In some embodiments, the antibody provided herein attenuates an IL-36γ activity by at least about 10%. In some embodiments, the antibody provided herein attenuates an IL-36γ activity by at least about 20%. In some embodiments, the antibody provided herein attenuates an IL-36γ activity by at least about 30%. In some embodiments, the antibody provided herein attenuates an IL-36γ activity by at least about 40%. In some embodiments, the antibody provided herein attenuates an IL-36γ activity by at least about 50%. In some embodiments, the antibody provided herein attenuates an IL-36γ activity by at least about 60%. In some embodiments, the antibody provided herein attenuates an IL-36γ activity by at least about 70%. In some embodiments, the antibody provided herein attenuates an IL-36γ activity by at least about 80%. In some embodiments, the antibody provided herein attenuates an IL-36γ activity by at least about 90%. In some embodiments, the antibody provided herein attenuates an IL-36γ activity by at least about 95%. In certain embodiments, the antibody described herein can attenuate (e.g., partially attenuate) an IL-36γ activity by at least about 15% to about 65%. In certain embodiments, the antibody described herein can attenuate (e.g., partially attenuate) an IL-36γ activity by at least about 20% to about 65%. In certain embodiments, the antibody described herein can attenuate (e.g., partially attenuate) an IL-36γ activity by at least about 30% to about 65%.

In specific embodiments, the attenuation of an IL-36γ activity is assessed by methods described herein. In specific embodiments, the attenuation of an IL-36γ activity is assessed by methods known to one of skill in the art. In certain embodiments, the attenuation of an IL-36γ activity is relative to the IL-36γ activity in the presence of stimulation without any anti-IL-36γ antibody. In certain embodiments, the attenuation of an IL-36γ activity is relative to the IL-36γ activity in the presence of stimulation with an unrelated antibody (e.g., an antibody that does not specifically bind to IL-36γ).

A non-limiting example of an IL-36γ activity is IL-36γ mediated signaling. Thus, in certain embodiments, the antibody described herein attenuates (e.g., partially attenuates) IL-36γ mediated signaling. In some embodiments, the antibody provided herein attenuates IL-36γ mediated signaling by at least about 10%. In some embodiments, the antibody provided herein attenuates IL-36γ mediated signaling by at least about 20%. In some embodiments, the antibody provided herein attenuates IL-36γ mediated signaling by at least about 30%. In some embodiments, the antibody provided herein attenuates IL-36γ mediated signaling by at least about 40%. In some embodiments, the antibody provided herein attenuates IL-36γ mediated signaling by at least about 50%. In some embodiments, the antibody provided herein attenuates IL-36γ mediated signaling by at least about 60%. In some embodiments, the antibody provided herein attenuates IL-36γ mediated signaling by at least about 70%. In some embodiments, the antibody provided herein attenuates IL-36γ mediated signaling by at least about 80%. In some embodiments, the antibody provided herein attenuates IL-36γ mediated signaling by at least about 90%. In some embodiments, the antibody provided herein attenuates IL-36γ mediated signaling by at least about 95%. In certain embodiments, the antibody described herein can attenuate (e.g., partially attenuate) IL-36γ mediated signaling by at least about 15% to about 65%. In certain embodiments, the antibody described herein can attenuate (e.g., partially attenuate) IL-36γ mediated signaling by at least about 20% to about 65%. In certain embodiments, the antibody described herein can attenuate (e.g., partially attenuate) IL-36γ mediated signaling by at least about 30% to about 65%.

Another non-limiting example of an IL-36γ activity is binding to IL-36 receptor. Thus, in certain embodiments, the antibody described herein attenuates (e.g., partially attenuates) the binding of IL-36γ to an IL-36 receptor. In some embodiments, the antibody provided herein attenuates the binding of IL-36γ to an IL-36 receptor by at least about 10%. In some embodiments, the antibody provided herein attenuates the binding of IL-36γ to an IL-36 receptor by at least about 20%. In some embodiments, the antibody provided herein attenuates the binding of IL-36γ to an IL-36 receptor by at least about 30%. In some embodiments, the antibody provided herein attenuates the binding of IL-36γ to an IL-36 receptor by at least about 40%. In some embodiments, the antibody provided herein attenuates the binding of IL-36γ to an IL-36 receptor by at least about 50%. In some embodiments, the antibody provided herein attenuates the binding of IL-36γ to an IL-36 receptor by at least about 60%. In some embodiments, the antibody provided herein attenuates the binding of IL-36γ to an IL-36 receptor by at least about 70%. In some embodiments, the antibody provided herein attenuates the binding of IL-36γ to an IL-36 receptor by at least about 80%. In some embodiments, the antibody provided herein attenuates the binding of IL-36γ to an IL-36 receptor by at least about 90%. In some embodiments, the antibody provided herein attenuates the binding of IL-36γ to an IL-36 receptor by at least about 95%. In certain embodiments, the antibody described herein can attenuate (e.g., partially attenuate) the binding of IL-36γ to an IL-36 receptor by at least about 15% to about 65%. In certain embodiments, the antibody described herein can attenuate (e.g., partially attenuate) the binding of IL-36γ to an IL-36 receptor by at least about 20% to about 65%. In certain embodiments, the antibody described herein can attenuate (e.g., partially attenuate) the binding of IL-36γ to an IL-36 receptor by at least about 30% to about 65%.

Another non-limiting example of an IL-36γ activity is signaling mediated by an IL-36 receptor. Thus, in certain embodiments, the antibody described herein attenuates (e.g., partially attenuates) IL-36 receptor mediated signaling. In some embodiments, the antibody provided herein attenuates IL-36 receptor mediated signaling by at least about 10%. In some embodiments, the antibody provided herein attenuates IL-36 receptor mediated signaling by at least about 20%. In some embodiments, the antibody provided herein attenuates IL-36 receptor mediated signaling by at least about 30%. In some embodiments, the antibody provided herein attenuates IL-36 receptor mediated signaling by at least about 40%. In some embodiments, the antibody provided herein attenuates IL-36 receptor mediated signaling by at least about 50%. In some embodiments, the antibody provided herein attenuates IL-36 receptor mediated signaling by at least about 60%. In some embodiments, the antibody provided herein attenuates IL-36 receptor mediated signaling by at least about 70%. In some embodiments, the antibody provided herein attenuates IL-36 receptor mediated signaling by at least about 80%. In some embodiments, the antibody provided herein attenuates IL-36 receptor mediated signaling by at least about 90%. In some embodiments, the antibody provided herein attenuates IL-36 receptor mediated signaling by at least about 95%. In certain embodiments, the antibody described herein can attenuate (e.g., partially attenuate) IL-36 receptor mediated signaling by at least about 15% to about 65%. In certain embodiments, the antibody described herein can attenuate (e.g., partially attenuate) IL-36 receptor mediated signaling by at least about 20% to about 65%. In certain embodiments, the antibody described herein can attenuate (e.g., partially attenuate) IL-36 receptor mediated signaling by at least about 30% to about 65%.

In specific embodiments, antibodies provided herein (e.g., any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c or an antigen-binding fragment thereof, or an antibody comprising CDRs of any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c) specifically bind to IL-36γ and inhibit the secretion of one or more cytokines and/or chemokines induced by IL-36γ. In some embodiments, the one or more cytokines and/or chemokines are selected from a group consisting of IL-8, IL-6, IL-10, TNFα, IL-1(3, CXCL1, CCLS, CCL20, CCL2, CCL3, CCL4, CXCL12, VEGF-A, IL-23, IL-36α, IL-36β, and IL-36γ.

For example, in one embodiment, an antibody provided herein specifically binds to IL-36γ and inhibits IL-8 secretion by at least about 5%. In one embodiment, an antibody provided herein specifically binds to IL-36γ and inhibits IL-8 secretion by at least about 10%. In one embodiment, an antibody provided herein specifically binds to IL-36γ and inhibits IL-8 secretion by at least about 15%. In one embodiment, an antibody provided herein specifically binds to IL-36γ and inhibits IL-8 secretion by at least about 20%. In one embodiment, an antibody provided herein specifically binds to IL-36γ and inhibits IL-8 secretion by at least about 25%. In one embodiment, an antibody provided herein specifically binds to IL-36γ and inhibits IL-8 secretion by at least about 30%. In one embodiment, an antibody provided herein specifically binds to IL-36γ and inhibits IL-8 secretion by at least about 35%. In one embodiment, an antibody provided herein specifically binds to IL-36γ and inhibits IL-8 secretion by at least about 40%. In one embodiment, an antibody provided herein specifically binds to IL-36γ and inhibits IL-8 secretion by at least about 45%. In one embodiment, an antibody provided herein specifically binds to IL-36γ and inhibits IL-8 secretion by at least about 50%. In one embodiment, an antibody provided herein specifically binds to IL-36γ and inhibits IL-8 secretion by at least about 55%. In one embodiment, an antibody provided herein specifically binds to IL-36γ and inhibits IL-8 secretion by at least about 60%. In one embodiment, an antibody provided herein specifically binds to IL-36γ and inhibits IL-8 secretion by at least about 65%. In one embodiment, an antibody provided herein specifically binds to IL-36γ and inhibits IL-8 secretion by at least about 70%. In one embodiment, an antibody provided herein specifically binds to IL-36γ and inhibits IL-8 secretion by at least about 75%. In one embodiment, an antibody provided herein specifically binds to IL-36γ and inhibits IL-8 secretion by at least about 80%. In one embodiment, an antibody provided herein specifically binds to IL-36γ and inhibits IL-8 secretion by at least about 85%. In one embodiment, an antibody provided herein specifically binds to IL-36γ and inhibits IL-8 secretion by at least about 90%. In one embodiment, an antibody provided herein specifically binds to IL-36γ and inhibits IL-8 secretion by at least about 95%. In one embodiment, an antibody provided herein specifically binds to IL-36γ and inhibits IL-8 secretion by at least about 96%. In one embodiment, an antibody provided herein specifically binds to IL-36γ and inhibits IL-8 secretion by at least about 97%. In one embodiment, an antibody provided herein specifically binds to IL-36γ and inhibits IL-8 secretion by at least about 98%. In one embodiment, an antibody provided herein specifically binds to IL-36γ and inhibits IL-8 secretion by at least about 99%. In some embodiments, the inhibition of IL-8 secretion is assessed by methods described herein. In other embodiments, the inhibition of IL-8 secretion is assessed by methods known to one of skill in the art. In a specific embodiment, the IL-8 secretion is inhibited relative to IL-8 secretion in the absence of anti-IL-36γ antibody. In other embodiments, the IL-8 secretion is inhibited relative to IL-8 secretion in the presence of an unrelated antibody (e.g., an antibody that does not specifically bind to IL-36γ).

In one embodiment, the antibody provided herein inhibits IL-8 secretion with an IC₅₀ of at most about 100 nM. In one embodiment, the antibody provided herein inhibits IL-8 secretion with an IC₅₀ of at most about 90 nM. In one embodiment, the antibody provided herein inhibits IL-8 secretion with an IC₅₀ of at most about 80 nM. In one embodiment, the antibody provided herein inhibits IL-8 secretion with an IC₅₀ of at most about 70 nM. In one embodiment, the antibody provided herein inhibits IL-8 secretion with an IC₅₀ of at most about 60 nM. In one embodiment, the antibody provided herein inhibits IL-8 secretion with an IC₅₀ of at most about 50 nM. In one embodiment, the antibody provided herein inhibits IL-8 secretion with an IC₅₀ of at most about 40 nM. In one embodiment, the antibody provided herein inhibits IL-8 secretion with an IC₅₀ of at most about 30 nM. In one embodiment, the antibody provided herein inhibits IL-8 secretion with an IC₅₀ of at most about 20 nM. In one embodiment, the antibody provided herein inhibits IL-8 secretion with an IC₅₀ of at most about 10 nM. In one embodiment, the antibody provided herein inhibits IL-8 secretion with an IC₅₀ of at most about 1 nM. In one embodiment, the antibody provided herein inhibits IL-8 secretion with an IC₅₀ of at most about 0.1 nM. In one embodiment, the antibody provided herein inhibits IL-8 secretion with an IC₅₀ of at most about 0.05 nM. In one embodiment, the antibody provided herein inhibits IL-8 secretion with an IC₅₀ of at most about 0.001 nM.

In one embodiment, the antibody provided herein inhibits IL-8 secretion with an IC₅₀ of at least about 100 nM. In one embodiment, the antibody provided herein inhibits IL-8 secretion with an IC₅₀ of at least about 90 nM. In one embodiment, the antibody provided herein inhibits IL-8 secretion with an IC₅₀ of at least about 80 nM. In one embodiment, the antibody provided herein inhibits IL-8 secretion with an IC₅₀ of at least about 70 nM. In one embodiment, the antibody provided herein inhibits IL-8 secretion with an IC₅₀ of at least about 60 nM. In one embodiment, the antibody provided herein inhibits IL-8 secretion with an IC₅₀ of at least about 50 nM. In one embodiment, the antibody provided herein inhibits IL-8 secretion with an IC₅₀ of at least about 40 nM. In one embodiment, the antibody provided herein inhibits IL-8 secretion with an IC₅₀ of at least about 30 nM. In one embodiment, the antibody provided herein inhibits IL-8 secretion with an IC₅₀ of at least about 20 nM. In one embodiment, the antibody provided herein inhibits IL-8 secretion with an IC₅₀ of at least about 10 nM. In one embodiment, the antibody provided herein inhibits IL-8 secretion with an IC₅₀ of at least about 1 nM. In one embodiment, the antibody provided herein inhibits IL-8 secretion with an IC₅₀ of at least about 0.1 nM. In one embodiment, the antibody provided herein inhibits IL-8 secretion with an IC₅₀ of at least about 0.05 nM. In one embodiment, the antibody provided herein inhibits IL-8 secretion with an IC₅₀ of at least about 0.001 nM. In specific embodiments, the IC₅₀ is assessed by methods described herein, for example, in Section 6 below. In other embodiments, the IC₅₀ is assessed by other methods known to one of skill in the art.

In certain embodiments, the antibody provided herein binds to IL-36γ and attenuates (e.g., partially attenuates) IL-36 receptor dimerization (i.e., heterodimerization between IL-36R (also known as IL-1Rrp2) and IL-1RAcP (also known as IL-1 receptor accessory protein)). In some embodiments, the antibody provided herein binds to IL-36γ and attenuates IL-36 receptor dimerization by at least about 10%. In some embodiments, the antibody provided herein binds to IL-36γ and attenuates IL-36 receptor dimerization by at least about 15%. In some embodiments, the antibody provided herein binds to IL-36γ and attenuates IL-36 receptor dimerization by at least about 20%. In some embodiments, the antibody provided herein binds to IL-36γ and attenuates IL-36 receptor dimerization by at least about 25%. In some embodiments, the antibody provided herein binds to IL-36γ and attenuates IL-36 receptor dimerization by at least about 30%. In some embodiments, the antibody provided herein binds to IL-36γ and attenuates IL-36 receptor dimerization by at least about 35%. In some embodiments, the antibody provided herein binds to IL-36γ and attenuates IL-36 receptor dimerization by at least about 40%. In some embodiments, the antibody provided herein binds to IL-36γ and attenuates IL-36 receptor dimerization by at least about 45%. In some embodiments, the antibody provided herein binds to IL-36γ and attenuates IL-36 receptor dimerization by at least about 50%. In some embodiments, the antibody provided herein binds to IL-36γ and attenuates IL-36 receptor dimerization by at least about 55%. In some embodiments, the antibody provided herein binds to IL-36γ and attenuates IL-36 receptor dimerization by at least about 60%. In some embodiments, the antibody provided herein binds to IL-36γ and attenuates IL-36 receptor dimerization by at least about 65%. In some embodiments, the antibody provided herein binds to IL-36γ and attenuates IL-36 receptor dimerization by at least about 70%. In some embodiments, the antibody provided herein binds to IL-36γ and attenuates IL-36 receptor dimerization by at least about 75%. In some embodiments, the antibody provided herein binds to IL-36γ and attenuates IL-36 receptor dimerization by at least about 80%. In some embodiments, the antibody provided herein binds to IL-36γ and attenuates IL-36 receptor dimerization by at least about 85%. In some embodiments, the antibody provided herein binds to IL-36γ and attenuates IL-36 receptor dimerization by at least about 90%. In some embodiments, the antibody provided herein binds to IL-36γ and attenuates IL-36 receptor dimerization by at least about 95%.

In certain embodiments, the antibody provided herein binds to IL-36γ and attenuates (e.g., partially attenuates) activation of mitogen-activated protein kinase (MAPK) pathways and/or nuclear factor kappa B (NF-κB) dependent transcription. In certain embodiments, the antibody provided herein binds to IL-36γ and attenuates activation of MAPK pathways and/or NF-κB dependent transcription by at least about 10%. In certain embodiments, the antibody provided herein binds to IL-36γ and attenuates activation of MAPK pathways and/or NF-κB dependent transcription by at least about 15%. In certain embodiments, the antibody provided herein binds to IL-36γ and attenuates activation of MAPK pathways and/or NF-κB dependent transcription by at least about 20%. In certain embodiments, the antibody provided herein binds to IL-36γ and attenuates activation of MAPK pathways and/or NF-κB dependent transcription by at least about 25%. In certain embodiments, the antibody provided herein binds to IL-36γ and attenuates activation of MAPK pathways and/or NF-κB dependent transcription by at least about 30%. In certain embodiments, the antibody provided herein binds to IL-36γ and attenuates activation of MAPK pathways and/or NF-κB dependent transcription by at least about 35%. In certain embodiments, the antibody provided herein binds to IL-36γ and attenuates activation of MAPK pathways and/or NF-κB dependent transcription by at least about 40%. In certain embodiments, the antibody provided herein binds to IL-36γ and attenuates activation of MAPK pathways and/or NF-κB dependent transcription by at least about 45%. In certain embodiments, the antibody provided herein binds to IL-36γ and attenuates activation of MAPK pathways and/or NF-κB dependent transcription by at least about 50%. In certain embodiments, the antibody provided herein binds to IL-36γ and attenuates activation of MAPK pathways and/or NF-κB dependent transcription by at least about 60%. In certain embodiments, the antibody provided herein binds to IL-36γ and attenuates activation of MAPK pathways and/or NF-κB dependent transcription by at least about 65%. In certain embodiments, the antibody provided herein binds to IL-36γ and attenuates activation of MAPK pathways and/or NF-κB dependent transcription by at least about 70%. In certain embodiments, the antibody provided herein binds to IL-36γ and attenuates activation of MAPK pathways and/or NF-κB dependent transcription by at least about 75%. In certain embodiments, the antibody provided herein binds to IL-36γ and attenuates activation of MAPK pathways and/or NF-κB dependent transcription by at least about 80%. In certain embodiments, the antibody provided herein binds to IL-36γ and attenuates activation of MAPK pathways and/or NF-κB dependent transcription by at least about 85%. In certain embodiments, the antibody provided herein binds to IL-36γ and attenuates activation of MAPK pathways and/or NF-κB dependent transcription by at least about 90%. In certain embodiments, the antibody provided herein binds to IL-36γ and attenuates activation of MAPK pathways and/or NF-κB dependent transcription by at least about 95%.

In yet another aspect, provided herein are antibodies that specifically bind to both IL-36α and IL-36γ and can modulate the activity and/or expression of IL-36α and/or IL-36γ (e.g., inhibit IL-36α and/or IL-36γ mediated signaling). In certain embodiments, dual antagonists against both IL-36α and IL-36γ are provided herein that are antibodies described herein that specifically bind to both IL-36α and IL-36γ and inhibit (including partially inhibit) at least one IL-36α activity and/or one IL-36γ activity.

An IL-36α activity can relate to any activity of IL-36α such as those known or described in the art. In certain embodiments, IL-36α activity and IL-36α signaling (or IL-36α mediated signaling) are used interchangeably herein. In certain aspects, IL-36α activity is induced by IL-36 receptor (e.g., IL-36α binding to IL-36 receptor). In certain embodiments, provided herein are antibodies that specifically bind to IL-36α and inhibit (or reduce) cytokine production. In some embodiments, the antibodies provided herein do not inhibit the binding of IL-36α to IL-36 receptor, but nevertheless inhibit or reduce the IL-36α mediated or IL-36 receptor mediated signaling. Similarly, an IL-36γ activity can relate to any activity of IL-36γ such as those known or described in the art. In certain embodiments, IL-36γ activity and IL-36γ signaling (or IL-36γ mediated signaling) are used interchangeably herein. In certain aspects, IL-36γ activity is induced by IL-36 receptor (e.g., IL-36γ binding to IL-36 receptor). In certain embodiments, provided herein are antibodies that specifically bind to IL-36γ and inhibit (or reduce) cytokine production. In some embodiments, the antibodies provided herein do not inhibit the binding of IL-36γ to IL-36 receptor, but nevertheless inhibit or reduce the IL-36γ mediated or IL-36 receptor mediated signaling.

In certain embodiments, the antibody described herein attenuates (e.g., partially attenuates) an IL-36α and/or IL-36γ activity. In some embodiments, the antibody provided herein attenuates an IL-36α and/or IL-36γ activity by at least about 10%. In some embodiments, the antibody provided herein attenuates an IL-36α and/or IL-36γ activity by at least about 20%. In some embodiments, the antibody provided herein attenuates an IL-36α and/or IL-36γ activity by at least about 30%. In some embodiments, the antibody provided herein attenuates an IL-36α and/or IL-36γ activity by at least about 40%. In some embodiments, the antibody provided herein attenuates an IL-36α and/or IL-36γ activity by at least about 50%. In some embodiments, the antibody provided herein attenuates an IL-36α and/or IL-36γ activity by at least about 60%. In some embodiments, the antibody provided herein attenuates an IL-36α and/or IL-36γ activity by at least about 70%. In some embodiments, the antibody provided herein attenuates an IL-36α and/or IL-36γ activity by at least about 80%. In some embodiments, the antibody provided herein attenuates an IL-36α and/or IL-36γ activity by at least about 90%. In some embodiments, the antibody provided herein attenuates an IL-36α and/or IL-36γ activity by at least about 95%. In certain embodiments, the antibody described herein can attenuate (e.g., partially attenuate) an IL-36α and/or IL-36γ activity by at least about 15% to about 65%. In certain embodiments, the antibody described herein can attenuate (e.g., partially attenuate) an IL-36α and/or IL-36γ activity by at least about 20% to about 65%. In certain embodiments, the antibody described herein can attenuate (e.g., partially attenuate) an IL-36α and/or IL-36γ activity by at least about 30% to about 65%.

In specific embodiments, the attenuation of an IL-36α and/or IL-36γ activity is assessed by methods described herein. In specific embodiments, the attenuation of an IL-36α and/or IL-36γ activity is assessed by methods known to one of skill in the art. In certain embodiments, the attenuation of an activity is relative to the activity in the presence of stimulation without any anti-IL-36α antibody or anti-IL-36γ antibody. In certain embodiments, the attenuation of an activity is relative to the activity in the presence of stimulation with an unrelated antibody (e.g., an antibody that does not specifically bind to IL-36α and/or IL-36γ).

A non-limiting example of an IL-36α and/or IL-36γ activity is IL-36α and/or IL-36γ mediated signaling. Thus, in certain embodiments, the antibody described herein attenuates (e.g., partially attenuates) IL-36α and/or IL-36γ mediated signaling. In some embodiments, the antibody provided herein attenuates IL-36α and/or IL-36γ mediated signaling by at least about 10%. In some embodiments, the antibody provided herein attenuates IL-36α and/or IL-36γ mediated signaling by at least about 20%. In some embodiments, the antibody provided herein attenuates IL-36α and/or IL-36γ mediated signaling by at least about 30%. In some embodiments, the antibody provided herein attenuates IL-36α and/or IL-36γ mediated signaling by at least about 40%. In some embodiments, the antibody provided herein attenuates IL-36α and/or IL-36γ mediated signaling by at least about 50%. In some embodiments, the antibody provided herein attenuates IL-36α and/or IL-36γ mediated signaling by at least about 60%. In some embodiments, the antibody provided herein attenuates IL-36α and/or IL-36γ mediated signaling by at least about 70%. In some embodiments, the antibody provided herein attenuates IL-36α and/or IL-36γ mediated signaling by at least about 80%. In some embodiments, the antibody provided herein attenuates IL-36α and/or IL-36γ mediated signaling by at least about 90%. In some embodiments, the antibody provided herein attenuates IL-36α and/or IL-36γ mediated signaling by at least about 95%. In certain embodiments, the antibody described herein can attenuate (e.g., partially attenuate) IL-36α and/or IL-36γ mediated signaling by at least about 15% to about 65%. In certain embodiments, the antibody described herein can attenuate (e.g., partially attenuate) IL-36α and/or IL-36γ mediated signaling by at least about 20% to about 65%. In certain embodiments, the antibody described herein can attenuate (e.g., partially attenuate) IL-36α and/or IL-36γ mediated signaling by at least about 30% to about 65%.

Another non-limiting example of an IL-36α and/or IL-36γ activity is binding to IL-36 receptor. Thus, in certain embodiments, the antibody described herein attenuates (e.g., partially attenuates) the binding of IL-36α and/or IL-36γ to an IL-36 receptor. In some embodiments, the antibody provided herein attenuates the binding of IL-36α and/or IL-36γ to an IL-36 receptor by at least about 10%. In some embodiments, the antibody provided herein attenuates the binding of IL-36α and/or IL-36γ to an IL-36 receptor by at least about 20%. In some embodiments, the antibody provided herein attenuates the binding of IL-36α and/or IL-36γ to an IL-36 receptor by at least about 30%. In some embodiments, the antibody provided herein attenuates the binding of IL-36α and/or IL-36γ to an IL-36 receptor by at least about 40%. In some embodiments, the antibody provided herein attenuates the binding of IL-36α and/or IL-36γ to an IL-36 receptor by at least about 50%. In some embodiments, the antibody provided herein attenuates the binding of IL-36α and/or IL-36γ to an IL-36 receptor by at least about 60%. In some embodiments, the antibody provided herein attenuates the binding of IL-36α and/or IL-36γ to an IL-36 receptor by at least about 70%. In some embodiments, the antibody provided herein attenuates the binding of IL-36α and/or IL-36γ to an IL-36 receptor by at least about 80%. In some embodiments, the antibody provided herein attenuates the binding of IL-36α and/or IL-36γ to an IL-36 receptor by at least about 90%. In some embodiments, the antibody provided herein attenuates the binding of IL-36α and/or IL-36γ to an IL-36 receptor by at least about 95%. In certain embodiments, the antibody described herein can attenuate (e.g., partially attenuate) the binding of IL-36α and/or IL-36γ to an IL-36 receptor by at least about 15% to about 65%. In certain embodiments, the antibody described herein can attenuate (e.g., partially attenuate) the binding of IL-36α and/or IL-36γ to an IL-36 receptor by at least about 20% to about 65%. In certain embodiments, the antibody described herein can attenuate (e.g., partially attenuate) the binding of IL-36α and/or IL-36γ to an IL-36 receptor by at least about 30% to about 65%.

Another non-limiting example of an IL-36α and/or IL-36γ activity is signaling mediated by an IL-36 receptor. Thus, in certain embodiments, the antibody described herein attenuates (e.g., partially attenuates) IL-36 receptor mediated signaling. In some embodiments, the antibody provided herein attenuates IL-36 receptor mediated signaling by at least about 10%. In some embodiments, the antibody provided herein attenuates IL-36 receptor mediated signaling by at least about 20%. In some embodiments, the antibody provided herein attenuates IL-36 receptor mediated signaling by at least about 30%. In some embodiments, the antibody provided herein attenuates IL-36 receptor mediated signaling by at least about 40%. In some embodiments, the antibody provided herein attenuates IL-36 receptor mediated signaling by at least about 50%. In some embodiments, the antibody provided herein attenuates IL-36 receptor mediated signaling by at least about 60%. In some embodiments, the antibody provided herein attenuates IL-36 receptor mediated signaling by at least about 70%. In some embodiments, the antibody provided herein attenuates IL-36 receptor mediated signaling by at least about 80%. In some embodiments, the antibody provided herein attenuates IL-36 receptor mediated signaling by at least about 90%. In some embodiments, the antibody provided herein attenuates IL-36 receptor mediated signaling by at least about 95%. In certain embodiments, the antibody described herein can attenuate (e.g., partially attenuate) IL-36 receptor mediated signaling by at least about 15% to about 65%. In certain embodiments, the antibody described herein can attenuate (e.g., partially attenuate) IL-36 receptor mediated signaling by at least about 20% to about 65%. In certain embodiments, the antibody described herein can attenuate (e.g., partially attenuate) IL-36 receptor mediated signaling by at least about 30% to about 65%.

In specific embodiments, antibodies provided herein (e.g., any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c or an antigen-binding fragment thereof, or an antibody comprising CDRs of any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c) specifically bind to IL-36α and IL-36γ and inhibit the secretion of one or more cytokines and/or chemokines induced by IL-36α and IL-36γ. In some embodiments, the one or more cytokines and/or chemokines are selected from a group consisting of IL-8, IL-6, IL-10, TNFα, IL-1β, CXCL1, CCL5, CCL20, CCL2, CCL3, CCL4, CXCL12, VEGF-A, IL-23, IL-36α, IL-36β, and IL-36γ.

In one embodiment, an antibody provided herein specifically binds to IL-36α and IL-36γ and inhibits IL-8 secretion by at least about 5%. In one embodiment, an antibody provided herein specifically binds to IL-36α and IL-36γ and inhibits IL-8 secretion by at least about 10%. In one embodiment, an antibody provided herein specifically binds to IL-36α and IL-36γ and inhibits IL-8 secretion by at least about 15%. In one embodiment, an antibody provided herein specifically binds to IL-36α and IL-36γ and inhibits IL-8 secretion by at least about 20%. In one embodiment, an antibody provided herein specifically binds to IL-36α and IL-36γ and inhibits IL-8 secretion by at least about 25%. In one embodiment, an antibody provided herein specifically binds to IL-36α and IL-36γ and inhibits IL-8 secretion by at least about 30%. In one embodiment, an antibody provided herein specifically binds to IL-36α and IL-36γ and inhibits IL-8 secretion by at least about 35%. In one embodiment, an antibody provided herein specifically binds to IL-36α and IL-36γ and inhibits IL-8 secretion by at least about 40%. In one embodiment, an antibody provided herein specifically binds to IL-36α and IL-36γ and inhibits IL-8 secretion by at least about 45%. In one embodiment, an antibody provided herein specifically binds to IL-36α and IL-36γ and inhibits IL-8 secretion by at least about 50%. In one embodiment, an antibody provided herein specifically binds to IL-36α and IL-36γ and inhibits IL-8 secretion by at least about 55%. In one embodiment, an antibody provided herein specifically binds to IL-36α and IL-36γ and inhibits IL-8 secretion by at least about 60%. In one embodiment, an antibody provided herein specifically binds to IL-36α and IL-36γ and inhibits IL-8 secretion by at least about 65%. In one embodiment, an antibody provided herein specifically binds to IL-36α and IL-36γ and inhibits IL-8 secretion by at least about 70%. In one embodiment, an antibody provided herein specifically binds to IL-36α and IL-36γ and inhibits IL-8 secretion by at least about 75%. In one embodiment, an antibody provided herein specifically binds to IL-36α and IL-36γ and inhibits IL-8 secretion by at least about 80%. In one embodiment, an antibody provided herein specifically binds to IL-36α and IL-36γ and inhibits IL-8 secretion by at least about 85%. In one embodiment, an antibody provided herein specifically binds to IL-36α and IL-36γ and inhibits IL-8 secretion by at least about 90%. In one embodiment, an antibody provided herein specifically binds to IL-36α and IL-36γ and inhibits IL-8 secretion by at least about 95%. In one embodiment, an antibody provided herein specifically binds to IL-36α and IL-36γ and inhibits IL-8 secretion by at least about 96%. In one embodiment, an antibody provided herein specifically binds to IL-36α and IL-36γ and inhibits IL-8 secretion by at least about 97%. In one embodiment, an antibody provided herein specifically binds to IL-36α and IL-36γ and inhibits IL-8 secretion by at least about 98%. In one embodiment, an antibody provided herein specifically binds to IL-36α and IL-36γ and inhibits IL-8 secretion by at least about 99%. In some embodiments, the inhibition of IL-8 secretion is assessed by methods described herein. In other embodiments, the inhibition of IL-8 secretion is assessed by methods known to one of skill in the art. In a specific embodiment, the IL-8 secretion is inhibited relative to IL-8 secretion in the absence of anti-IL-36α and IL-36γ antibody. In other embodiments, the IL-8 secretion is inhibited relative to IL-8 secretion in the presence of an unrelated antibody (e.g., an antibody that does not specifically bind to IL-36α and IL-36γ).

In one embodiment, the antibody provided herein inhibits IL-8 secretion with an IC₅₀ of at most about 100 nM. In one embodiment, the antibody provided herein inhibits IL-8 secretion with an IC₅₀ of at most about 90 nM. In one embodiment, the antibody provided herein inhibits IL-8 secretion with an IC₅₀ of at most about 80 nM. In one embodiment, the antibody provided herein inhibits IL-8 secretion with an IC₅₀ of at most about 70 nM. In one embodiment, the antibody provided herein inhibits IL-8 secretion with an IC₅₀ of at most about 60 nM. In one embodiment, the antibody provided herein inhibits IL-8 secretion with an IC₅₀ of at most about 50 nM. In one embodiment, the antibody provided herein inhibits IL-8 secretion with an IC₅₀ of at most about 40 nM. In one embodiment, the antibody provided herein inhibits IL-8 secretion with an IC₅₀ of at most about 30 nM. In one embodiment, the antibody provided herein inhibits IL-8 secretion with an IC₅₀ of at most about 20 nM. In one embodiment, the antibody provided herein inhibits IL-8 secretion with an IC₅₀ of at most about 10 nM. In one embodiment, the antibody provided herein inhibits IL-8 secretion with an IC₅₀ of at most about 1 nM. In one embodiment, the antibody provided herein inhibits IL-8 secretion with an IC₅₀ of at most about 0.1 nM. In one embodiment, the antibody provided herein inhibits IL-8 secretion with an IC₅₀ of at most about 0.05 nM. In one embodiment, the antibody provided herein inhibits IL-8 secretion with an IC₅₀ of at most about 0.001 nM.

In one embodiment, the antibody provided herein inhibits IL-8 secretion with an IC₅₀ of at least about 100 nM. In one embodiment, the antibody provided herein inhibits IL-8 secretion with an IC₅₀ of at least about 90 nM. In one embodiment, the antibody provided herein inhibits IL-8 secretion with an IC₅₀ of at least about 80 nM. In one embodiment, the antibody provided herein inhibits IL-8 secretion with an IC₅₀ of at least about 70 nM. In one embodiment, the antibody provided herein inhibits IL-8 secretion with an IC₅₀ of at least about 60 nM. In one embodiment, the antibody provided herein inhibits IL-8 secretion with an IC₅₀ of at least about 50 nM. In one embodiment, the antibody provided herein inhibits IL-8 secretion with an IC50 of at least about 40 nM. In one embodiment, the antibody provided herein inhibits IL-8 secretion with an IC50 of at least about 30 nM. In one embodiment, the antibody provided herein inhibits IL-8 secretion with an IC50 of at least about 20 nM. In one embodiment, the antibody provided herein inhibits IL-8 secretion with an IC₅₀ of at least about 10 nM. In one embodiment, the antibody provided herein inhibits IL-8 secretion with an IC₅₀ of at least about 1 nM. In one embodiment, the antibody provided herein inhibits IL-8 secretion with an IC₅₀ of at least about 0.1 nM. In one embodiment, the antibody provided herein inhibits IL-8 secretion with an IC₅₀ of at least about 0.05 nM. In one embodiment, the antibody provided herein inhibits IL-8 secretion with an IC50 of at least about 0.001 nM. In specific embodiments, the IC₅₀ is assessed by methods described herein, for example, in Section 6 below. In other embodiments, the IC₅₀ is assessed by other methods known to one of skill in the art.

In certain embodiments, the antibody provided herein binds to IL-36α and IL-36γ and attenuates (e.g., partially attenuates) IL-36 receptor dimerization (i.e., heterodimerization between IL-36R (also known as IL-1Rrp2) and IL-1RAcP (also known as IL-1 receptor accessory protein)). In some embodiments, the antibody provided herein binds to IL-36α and IL-36γ and attenuates IL-36 receptor dimerization by at least about 10%. In some embodiments, the antibody provided herein binds to IL-36α and IL-36γ and attenuates IL-36 receptor dimerization by at least about 15%. In some embodiments, the antibody provided herein binds to IL-36α and IL-36γ and attenuates IL-36 receptor dimerization by at least about 20%. In some embodiments, the antibody provided herein binds to IL-36α and IL-36γ and attenuates IL-36 receptor dimerization by at least about 25%. In some embodiments, the antibody provided herein binds to IL-36α and IL-36γ and attenuates IL-36 receptor dimerization by at least about 30%. In some embodiments, the antibody provided herein binds to IL-36α and IL-36γ and attenuates IL-36 receptor dimerization by at least about 35%. In some embodiments, the antibody provided herein binds to IL-36α and IL-36γ and attenuates IL-36 receptor dimerization by at least about 40%. In some embodiments, the antibody provided herein binds to IL-36α and IL-36γ and attenuates IL-36 receptor dimerization by at least about 45%. In some embodiments, the antibody provided herein binds to IL-36α and IL-36γ and attenuates IL-36 receptor dimerization by at least about 50%. In some embodiments, the antibody provided herein binds to IL-36α and IL-36γ and attenuates IL-36 receptor dimerization by at least about 55%. In some embodiments, the antibody provided herein binds to IL-36α and IL-36γ and attenuates IL-36 receptor dimerization by at least about 60%. In some embodiments, the antibody provided herein binds to IL-36α and IL-36γ and attenuates IL-36 receptor dimerization by at least about 65%. In some embodiments, the antibody provided herein binds to IL-36α and IL-36γ and attenuates IL-36 receptor dimerization by at least about 70%. In some embodiments, the antibody provided herein binds to IL-36α and IL-36γ and attenuates IL-36 receptor dimerization by at least about 75%. In some embodiments, the antibody provided herein binds to IL-36α and IL-36γ and attenuates IL-36 receptor dimerization by at least about 80%. In some embodiments, the antibody provided herein binds to IL-36α and IL-36γ and attenuates IL-36 receptor dimerization by at least about 85%. In some embodiments, the antibody provided herein binds to IL-36α and IL-36γ and attenuates IL-36 receptor dimerization by at least about 90%. In some embodiments, the antibody provided herein binds to IL-36α and IL-36γ and attenuates IL-36 receptor dimerization by at least about 95%.

In certain embodiments, the antibody provided herein binds to IL-36α and IL-36γ and attenuates (e.g., partially attenuates) activation of mitogen-activated protein kinase (MAPK) pathways and/or nuclear factor kappa B (NF-κB) dependent transcription. In certain embodiments, the antibody provided herein binds to IL-36α and IL-36γ and attenuates activation of MAPK pathways and/or NF-κB dependent transcription by at least about 10%. In certain embodiments, the antibody provided herein binds to IL-36α and IL-36γ and attenuates activation of MAPK pathways and/or NF-κB dependent transcription by at least about 15%. In certain embodiments, the antibody provided herein binds to IL-36α and IL-36γ and attenuates activation of MAPK pathways and/or NF-κB dependent transcription by at least about 20%. In certain embodiments, the antibody provided herein binds to IL-36α and IL-36γ and attenuates activation of MAPK pathways and/or NF-κB dependent transcription by at least about 25%. In certain embodiments, the antibody provided herein binds to IL-36α and IL-36γ and attenuates activation of MAPK pathways and/or NF-κB dependent transcription by at least about 30%. In certain embodiments, the antibody provided herein binds to IL-36α and IL-36γ and attenuates activation of MAPK pathways and/or NF-κB dependent transcription by at least about 35%. In certain embodiments, the antibody provided herein binds to IL-36α and IL-36γ and attenuates activation of MAPK pathways and/or NF-κB dependent transcription by at least about 40%. In certain embodiments, the antibody provided herein binds to IL-36α and IL-36γ and attenuates activation of MAPK pathways and/or NF-κB dependent transcription by at least about 45%. In certain embodiments, the antibody provided herein binds to IL-36α and IL-36γ and attenuates activation of MAPK pathways and/or NF-κB dependent transcription by at least about 50%. In certain embodiments, the antibody provided herein binds to IL-36α and IL-36γ and attenuates activation of MAPK pathways and/or NF-κB dependent transcription by at least about 60%. In certain embodiments, the antibody provided herein binds to IL-36α and IL-36γ and attenuates activation of MAPK pathways and/or NF-κB dependent transcription by at least about 65%. In certain embodiments, the antibody provided herein binds to IL-36α and IL-36γ and attenuates activation of MAPK pathways and/or NF-κB dependent transcription by at least about 70%. In certain embodiments, the antibody provided herein binds to IL-36α and IL-36γ and attenuates activation of MAPK pathways and/or NF-κB dependent transcription by at least about 75%. In certain embodiments, the antibody provided herein binds to IL-36α and IL-36γ and attenuates activation of MAPK pathways and/or NF-κB dependent transcription by at least about 80%. In certain embodiments, the antibody provided herein binds to IL-36α and IL-36γ and attenuates activation of MAPK pathways and/or NF-κB dependent transcription by at least about 85%. In certain embodiments, the antibody provided herein binds to IL-36α and IL-36γ and attenuates activation of MAPK pathways and/or NF-κB dependent transcription by at least about 90%. In certain embodiments, the antibody provided herein binds to IL-36α and IL-36γ and attenuates activation of MAPK pathways and/or NF-κB dependent transcription by at least about 95%.

In some embodiments, the antibody or antigen binding fragment thereof provided herein are selected from a group consisting of the antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c as described in Section 6 below and antigen binding fragments thereof.

In some embodiments, the antibody provided herein comprises one or more CDR regions from antibody 144D464A.

In some embodiments, the antibody comprises a CDR H1 having an amino acid sequence of the CDR H1 contained in SEQ ID NO: 23. In some embodiments, the antibody comprises a CDR H2 having an amino acid sequence of the CDR H2 contained in SEQ ID NO: 23. In some embodiments, the antibody comprises a CDR H3 having an amino acid sequence of the CDR H3 contained in SEQ ID NO: 23. In some embodiments, the antibody comprises a CDR L1 having an amino acid sequence of the CDR L1 contained in SEQ ID NO: 51. In some embodiments, the antibody comprises a CDR L2 having an amino acid sequence of the CDR L2 contained in SEQ ID NO: 51. In some embodiments, the antibody comprises a CDR L3 having an amino acid sequence of the CDRL3 contained in SEQ ID NO: 51.

In some embodiments, the antibody comprises a CDR H1 and a CDR H2 having amino acid sequences of the CDR H1 and the CDR H2 contained in SEQ ID NO: 23. In some embodiments, the antibody comprises a CDR H1 and a CDR H3 having amino acid sequences of the CDR H1 and the CDR H3 contained in SEQ ID NO: 23. In some embodiments, the antibody comprises a CDR H2 and a CDR H3 having amino acid sequences of the CDR H2 and the CDR H3 contained in SEQ ID NO: 23. In some embodiments, the antibody comprises a CDR L1 and a CDR L2 having amino acid sequences of the CDR L1 and the CDR L2 contained in SEQ ID NO: 51. In some embodiments, the antibody comprises a CDR L1 and a CDR L3 having amino acid sequences of the CDR L1 and the CDR L3 contained in SEQ ID NO: 51. In some embodiments, the antibody comprises a CDR L2 and a CDR L3 having amino acid sequences of the CDR L2 and the CDR L3 contained in SEQ ID NO: 51.

In some embodiments, the antibody comprises a CDR H1 and a CDR L1 having amino acid sequences of the CDR H1 and the CDR L1 contained in SEQ ID NO: 23 and SEQ ID NO: 51 respectively. In some embodiments, the antibody comprises a CDR H1 and a CDR L2 having amino acid sequences of the CDR H1 and the CDR L2 contained in SEQ ID NO: 23 and SEQ ID NO: 51 respectively. In some embodiments, the antibody comprises a CDR H1 and a CDR L3 having amino acid sequences of the CDR H1 and the CDR L3 contained in SEQ ID NO: 23 and SEQ ID NO: 51 respectively. In some embodiments, the antibody comprises a CDR H2 and a CDR L1 having amino acid sequences of the CDR H2 and the CDR L1 contained in SEQ ID NO: 23 and SEQ ID NO: 51 respectively. In some embodiments, the antibody comprises a CDR H2 and a CDR L2 having amino acid sequences of the CDR H2 and the CDR L2 contained in SEQ ID NO: 23 and SEQ ID NO: 51 respectively. In some embodiments, the antibody comprises a CDR H2 and a CDR L3 having amino acid sequences of the CDR H2 and the CDR L3 contained in SEQ ID NO: 23 and SEQ ID NO: 51 respectively. In some embodiments, the antibody comprises a CDR H3 and a CDR L1 having amino acid sequences of the CDR H3 and the CDR L1 contained in SEQ ID NO: 23 and SEQ ID NO: 51 respectively. In some embodiments, the antibody comprises a CDR H3 and a CDR L2 having amino acid sequences of the CDR H3 and the CDR L2 contained in SEQ ID NO: 23 and SEQ ID NO: 51 respectively. In some embodiments, the antibody comprises a CDR H3 and a CDR L3 having amino acid sequences of the CDR H3 and the CDR L3 contained in SEQ ID NO: 23 and SEQ ID NO: 51 respectively.

In some embodiments, the antibody comprises a CDR H1, a CDR H2, and a CDR H3 having amino acid sequences of the CDR H1, the CDR H2, and the CDR H3 contained in SEQ ID NO: 23. In some embodiments, the antibody comprises a CDR L1, a CDR L2, and a CDR L3 having amino acid sequences of the CDR L1, the CDR L2, and the CDR L3 contained in SEQ ID NO: 51.

In some embodiments, the antibody comprises a CDR H1, a CDR H2, and a CDR L1 having amino acid sequences of the CDR H1, the CDR H2, and the CDR L1 contained in SEQ ID NO: 23 and SEQ ID NO: 51 respectively. In some embodiments, the antibody comprises a CDR H1, a CDR H2, and a CDR L2 having amino acid sequences of the CDR H1, the CDR H2, and the CDR L2 contained in SEQ ID NO: 23 and SEQ ID NO: 51 respectively. In some embodiments, the antibody comprises a CDR H1, a CDR H2, and a CDR L3 having amino acid sequences of the CDR H1, the CDR H2, and the CDR L3 contained in SEQ ID NO: 23 and SEQ ID NO: 51 respectively.

In some embodiments, the antibody comprises a CDR H1, a CDR H3, and a CDR L1 having amino acid sequences of the CDR H1, the CDR H3, and the CDR L1 contained in SEQ ID NO: 23 and SEQ ID NO: 51 respectively. In some embodiments, the antibody comprises a CDR H1, a CDR H3, and a CDR L2 having amino acid sequences of the CDR H1, the CDR H3, and the CDR L2 contained in SEQ ID NO: 23 and SEQ ID NO: 51 respectively. In some embodiments, the antibody comprises a CDR H1, a CDR H3, and a CDR L3 having amino acid sequences of the CDR H1, the CDR H3, and the CDR L3 contained in SEQ ID NO: 23 and SEQ ID NO: 51 respectively.

In some embodiments, the antibody comprises a CDR H2, a CDR H3, and a CDR L1 having amino acid sequences of the CDR H2, the CDR H3, and the CDR L1 contained in SEQ ID NO: 23 and SEQ ID NO: 51 respectively. In some embodiments, the antibody comprises a CDR H2, a CDR H3, and a CDR L2 having amino acid sequences of the CDR H2, the CDR H3, and the CDR L2 contained in SEQ ID NO: 23 and SEQ ID NO: 51 respectively. In some embodiments, the antibody comprises a CDR H2, a CDR H3, and a CDR L3 having amino acid sequences of the CDR H2, the CDR H3, and the CDR L3 contained in SEQ ID NO: 23 and SEQ ID NO: 51 respectively.

In some embodiments, the antibody comprises a CDR H1, a CDR L1, and a CDR L2 having amino acid sequences of the CDR H1, the CDR L1, and the CDR L2 contained in SEQ ID NO: 23 and SEQ ID NO: 51 respectively. In some embodiments, the antibody comprises a CDR H1, a CDR L1, and a CDR L3 having amino acid sequences of the CDR H1, the CDR L1, and the CDR L3 contained in SEQ ID NO: 23 and SEQ ID NO: 51 respectively. In some embodiments, the antibody comprises a CDR H1, a CDR L2, and a CDR L3 having amino acid sequences of the CDR H1, the CDR L2, and the CDR L3 contained in SEQ ID NO: 23 and SEQ ID NO: 51 respectively.

In some embodiments, the antibody comprises a CDR H2, a CDR L1, and a CDR L2 having amino acid sequences of the CDR H2, the CDR L1, and the CDR L2 contained in SEQ ID NO: 23 and SEQ ID NO: 51 respectively. In some embodiments, the antibody comprises a CDR H2, a CDR Ll, and a CDR L3 having amino acid sequences of the CDR H2, the CDR L1, and the CDR L3 contained in SEQ ID NO: 23 and SEQ ID NO: 51 respectively. In some embodiments, the antibody comprises a CDR H2, a CDR L2, and a CDR L3 having amino acid sequences of the CDR H2, the CDR L2, and the CDR L3 contained in SEQ ID NO: 23 and SEQ ID NO: 51 respectively.

In some embodiments, the antibody comprises a CDR H3, a CDR L1, and a CDR L2 having amino acid sequences of the CDR H3, the CDR L1, and the CDR L2 contained in SEQ ID NO: 23 and SEQ ID NO: 51 respectively. In some embodiments, the antibody comprises a CDR H3, a CDR L1, and a CDR L3 having amino acid sequences of the CDR H3, the CDR L1, and the CDR L3 contained in SEQ ID NO: 23 and SEQ ID NO: 51 respectively. In some embodiments, the antibody comprises a CDR H3, a CDR L2, and a CDR L3 having amino acid sequences of the CDR H3, the CDR L2, and the CDR L3 contained in SEQ ID NO: 23 and SEQ ID NO: 51 respectively.

In some embodiments, the antibody comprises a CDR H3, a CDR Ll, a CDR L2 and a CDR L3 having amino acid sequences of the CDR H3, the CDR L1, the CDR L2 and the CDR L3 contained in SEQ ID NO: 23 and SEQ ID NO: 51 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 23 and SEQ ID NO: 51 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 23 and SEQ ID NO: 51 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 23 and SEQ ID NO: 51 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 23 and SEQ ID NO: 51 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 23 and SEQ ID NO: 51 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 23 and SEQ ID NO: 51 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 23 and SEQ ID NO: 51 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 23 and SEQ ID NO: 51 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 23 and SEQ ID NO: 51 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 23 and SEQ ID NO: 51 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 23 and SEQ ID NO: 51 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 23 and SEQ ID NO: 51 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 23 and SEQ ID NO: 51 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 23 and SEQ ID NO: 51 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 23 and SEQ ID NO: 51 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 23 and SEQ ID NO: 51 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 23 and SEQ ID NO: 51 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 23 and SEQ ID NO: 51 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 23 and SEQ ID NO: 51 respectively. In some embodiments, the antibody comprises a CDR H1, a CDR H2, a CDR H3, a CDR Ll, and a CDR L2 having amino acid sequences of the CDR H1, the CDR H2, the CDR H3, the CDR L1, and the CDR L2 contained in SEQ ID NO: 23 and SEQ ID NO: 51 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 23 and SEQ ID NO: 51 respectively.

As described above, CDR regions are well known to those skilled in the art and have been defined by well-known numbering systems. The residues from each of these hypervariable regions or CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 68. In some embodiments, the antibody comprises a CDR H2 of SEQ ID NO: 69. In some embodiments, the antibody comprises a CDR H3 of SEQ ID NO: 70. In some embodiments, the antibody comprises a CDR L1 of SEQ ID NO: 83. In some embodiments, the antibody comprises a CDR L2 of SEQ ID NO: 84. In other embodiments, the antibody comprises a CDR L3 of SEQ ID NO: 85.

In some embodiments, the antibody comprises a CDR H1 of SEQ ID NO: 68 and a CDR H2 of SEQ ID NO: 69. In some embodiments, the antibody comprises a CDR H1 of SEQ ID NO: 68 and a CDR H3 of SEQ ID NO: 70. In some embodiments, the antibody comprises a CDR H2 of SEQ ID NO: 69 and a CDR H3 of SEQ ID NO: 70. In some embodiments, the antibody comprises a CDR L1 of SEQ ID NO: 83 and a CDR L2 of SEQ ID NO: 84. In some embodiments, the antibody comprises a CDR L1 of SEQ ID NO: 83 and a CDR L3 of SEQ ID NO: 85. In some embodiments, the antibody comprises a CDR L2 of SEQ ID NO: 84 and a CDR L3 of SEQ ID NO: 85.

In some embodiments, the antibody comprises a CDR H1 of SEQ ID NO: 68 and a CDR L1 of SEQ ID NO: 83. In some embodiments, the antibody comprises a CDR H1 of SEQ ID NO: 68 and a CDR L2 of SEQ ID NO: 84. In some embodiments, the antibody comprises a CDR H1 of SEQ ID NO: 68 and a CDR L3 of SEQ ID NO: 85. In some embodiments, the antibody comprises a CDR H2 of SEQ ID NO: 69 and a CDR L1 of SEQ ID NO: 83. In some embodiments, the antibody comprises a CDR H2 of SEQ ID NO: 69 and a CDR L2 of SEQ ID NO: 84. In some embodiments, the Ab comprises a CDR H2 of SEQ ID NO: 69 and a CDRL3 of SEQ ID NO: 85. In some embodiments, the antibody comprises a CDR H3 of SEQ ID NO: 70 and a CDR L1 of SEQ ID NO: 83. In some embodiments, the antibody comprises a CDR H3 of SEQ ID NO: 70 and a CDR L2 of SEQ ID NO: 84. In some embodiments, the antibody comprises a CDR H3 of SEQ ID NO: 70 and a CDR L3 of SEQ ID NO: 85.

In some embodiments, the antibody comprises a CDR H1 of SEQ ID NO: 68, a CDR H2 of SEQ ID NO: 69, and a CDR H3 of SEQ ID NO: 70. In some embodiments, the antibody comprises a CDR L1 of SEQ ID NO: 83, a CDR L2 of SEQ ID NO: 84, and a CDR L3 of SEQ ID NO: 85.

In some embodiments, the antibody comprises a CDR H1 of SEQ ID NO: 68, a CDR H2 of SEQ ID NO: 69, and a CDR L1 of SEQ ID NO: 83. In some embodiments, the antibody comprises a CDR H1 of SEQ ID NO: 68, a CDR H2 of SEQ ID NO: 69, and a CDR L2 of SEQ ID NO: 84. In some embodiments, the antibody comprises a CDR H1 of SEQ ID NO: 68, a CDR H2 of SEQ ID NO: 69, and a CDR L3 of SEQ ID NO: 85. In some embodiments, the antibody comprises a CDR H1 of SEQ ID NO: 68, a CDR H3 of SEQ ID NO: 70, and a CDR L1 of SEQ ID NO: 83. In some embodiments, the antibody comprises a CDR H1 of SEQ ID NO: 68, a CDR H3 of SEQ ID NO: 70, and a CDR L2 of SEQ ID NO: 84. In some embodiments, the antibody comprises a CDR H1 of SEQ ID NO: 68, a CDR H3 of SEQ ID NO: 70, and a CDR L3 of SEQ ID NO: 85. In some embodiments, the antibody comprises a CDR H2 of SEQ ID NO: 69, a CDR H3 of SEQ ID NO: 70, and a CDR L1 of SEQ ID NO: 83. In some embodiments, the antibody comprises a CDR H2 of SEQ ID NO: 69, a CDR H3 of SEQ ID NO: 70, and a CDR L2 of SEQ ID NO: 84. In some embodiments, the antibody comprises a CDR H2 of SEQ ID NO: 69, a CDR H3 of SEQ ID NO: 70, and a CDR L3 of SEQ ID NO: 85.

In some embodiments, the antibody comprises a CDR H1 of SEQ ID NO: 68, a CDR L1 of SEQ ID NO: 83, and a CDR L2 of SEQ ID NO: 84. In some embodiments, the antibody comprises a CDR H1 of SEQ ID NO: 68, a CDR L1 of SEQ ID NO: 83, and a CDR L3 of SEQ ID NO: 85. In some embodiments, the antibody comprises a CDR H1 of SEQ ID NO: 68, a CDR L2 of SEQ ID NO: 84, and a CDR L3 of SEQ ID NO: 85. In some embodiments, the antibody comprises a CDR H2 of SEQ ID NO: 69, a CDR L1 of SEQ ID NO: 83, and a CDR L2 of SEQ ID NO: 84. In some embodiments, the antibody comprises a CDR H2 of SEQ ID NO: 69, a CDR L1 of SEQ ID NO: 83, and a CDR L3 of SEQ ID NO: 85. In some embodiments, the antibody comprises a CDR H2 of SEQ ID NO: 69, a CDR L2 of SEQ ID NO: 84, and a CDR L3 of SEQ ID NO: 85. In some embodiments, the antibody comprises a CDR H3 of SEQ ID NO: 70, a CDR L1 of SEQ ID NO: 83, and a CDR L2 of SEQ ID NO: 84. In some embodiments, the antibody comprises a CDR H3 of SEQ ID NO: 70, a CDR L1 of SEQ ID NO: 83, and a CDR L3 of SEQ ID NO: 85. In some embodiments, the antibody comprises a CDR H3 of SEQ ID NO: 70, a CDR L2 of SEQ ID NO: 84, and a CDR L3 of SEQ ID NO: 85.

In some embodiments, the antibody comprises a CDR H3 of SEQ ID NO: 70, a CDR L1 of SEQ ID NO: 83, a CDR L2 of SEQ ID NO: 84, and a CDR L3 of SEQ ID NO: 85. In some embodiments, the antibody comprises a CDR H2 of SEQ ID NO: 69, a CDR L1 of SEQ ID NO: 83, a CDR L2 of SEQ ID NO: 84, and a CDR L3 of SEQ ID NO: 85. In some embodiments, the antibody comprises a CDR H1 of SEQ ID NO: 68, a CDR L1 of SEQ ID NO: 83, a CDR L2 of SEQ ID NO: 84, and a CDR L3 of SEQ ID NO: 85. In some embodiments, the antibody comprises a CDR H1 of SEQ ID NO: 68, a CDR H2 of SEQ ID NO: 69, a CDR H3 of SEQ ID NO: 70, and a CDR L3 of SEQ ID NO: 85. In some embodiments, the antibody comprises a CDR H1 of SEQ ID NO: 68, a CDR H2 of SEQ ID NO: 69, a CDR H3 of SEQ ID NO: 70, and a CDR L2 of SEQ ID NO: 84. In some embodiments, the antibody comprises a CDR H1 of SEQ ID NO: 68, a CDR H2 of SEQ ID NO: 69, a CDR H3 of SEQ ID NO: 70, and a CDR L1 of SEQ ID NO: 83.

In some embodiments, the antibody comprises a CDR H2 of SEQ ID NO: 69, a CDR H3 of SEQ ID NO: 70, a CDR L2 of SEQ ID NO: 84, and a CDR L3 of SEQ ID NO: 85. In some embodiments, the antibody comprises a CDR H2 of SEQ ID NO: 69, a CDR H3 of SEQ ID NO: 70, a CDR L1 of SEQ ID NO: 83, and a CDR L3 of SEQ ID NO: 85. In some embodiments, the antibody comprises a CDR H2 of SEQ ID NO: 69, a CDR H3 of SEQ ID NO: 70, a CDR L1 of SEQ ID NO: 83, and a CDR L2 of SEQ ID NO: 84. In some embodiments, the antibody comprises a CDR H1 of SEQ ID NO: 68, a CDR H3 of SEQ ID NO: 70, a CDR L2 of SEQ ID NO: 84, and a CDR L3 of SEQ ID NO: 85. In some embodiments, the antibody comprises a CDR H1 of SEQ ID NO: 68, a CDR H3 of SEQ ID NO: 70, a CDR L1 of SEQ ID NO: 83, and a CDR L3 of SEQ ID NO: 85. In some embodiments, the antibody comprises a CDR H1 of SEQ ID NO: 68, a CDR H3 of SEQ ID NO: 70, a CDR L1 of SEQ ID NO: 83, and a CDR L2 of SEQ ID NO: 84. In some embodiments, the antibody comprises a CDR H1 of SEQ ID NO: 68, a CDR H2 of SEQ ID NO: 69, a CDR L2 of SEQ ID NO: 84, and a CDR L3 of SEQ ID NO: 85. In some embodiments, the antibody comprises a CDR H1 of SEQ ID NO: 68, a CDR H2 of SEQ ID NO: 69, a CDR L1 of SEQ ID NO: 83, and a CDR L3 of SEQ ID NO: 85. In some embodiments, the antibody comprises a CDR H1 of SEQ ID NO: 68, a CDR H2 of SEQ ID NO: 69, a CDR L1 of SEQ ID NO: 83, and a CDR L2 of SEQ ID NO: 84.

In some embodiments, the antibody comprises a CDR H1 of SEQ ID NO: 68, a CDR H2 of SEQ ID NO: 69, a CDR H3 of SEQ ID NO: 70, a CDR L2 of SEQ ID NO: 84, and a CDR L3 of SEQ ID NO: 85. In some embodiments, the antibody comprises a CDR H1 of SEQ ID NO: 68, a CDR H2 of SEQ ID NO: 69, a CDR H3 of SEQ ID NO: 70, a CDR L1 of SEQ ID NO: 83, and a CDR L3 of SEQ ID NO: 85. In some embodiments, the antibody comprises a CDR H1 of SEQ ID NO: 68, a CDR H2 of SEQ ID NO: 69, a CDR H3 of SEQ ID NO: 70, a CDR L1 of SEQ ID NO: 83, and a CDR L2 of SEQ ID NO: 84. In some embodiments, the antibody comprises a CDR H2 of SEQ ID NO: 69, a CDR H3 of SEQ ID NO: 70, a CDR L1 of SEQ ID NO: 83, a CDR L2 of SEQ ID NO: 84, and a CDR L3 of SEQ ID NO: 85. In some embodiments, the antibody comprises a CDR H1 of SEQ ID NO: 68, a CDR H3 of SEQ ID NO: 70, a CDR L1 of SEQ ID NO: 83, a CDR L2 of SEQ ID NO: 84, and a CDR L3 of SEQ ID NO: 85. In some embodiments, the antibody comprises a CDR H1 of SEQ ID NO: 68, a CDR H2 of SEQ ID NO: 69, a CDR L1 of SEQ ID NO: 83, a CDR L2 of SEQ ID NO: 84, and a CDR L3 of SEQ ID NO: 85.

In a specific embodiment, the antibody comprises a CDR H1 of SEQ ID NO: 68, a CDR H2 of SEQ ID NO: 69, a CDR H3 of SEQ ID NO: 70, a CDR L1 of SEQ ID NO: 83, a CDR L2 of SEQ ID NO: 84, and a CDR L3 of SEQ ID NO: 85.

In some embodiments, the antibody provided herein comprises one or more CDR regions from antibody 144L249B.

In some embodiments, the antibody comprises a CDR H1 having an amino acid sequence of the CDR H1 contained in SEQ ID NO: 27. In some embodiments, the antibody comprises a CDR H2 having an amino acid sequence of the CDR H2 contained in SEQ ID NO: 27. In some embodiments, the antibody comprises a CDR H3 having an amino acid sequence of the CDR H3 contained in SEQ ID NO: 27. In some embodiments, the antibody comprises a CDR L1 having an amino acid sequence of the CDR L1 contained in SEQ ID NO: 55. In some embodiments, the antibody comprises a CDR L2 having an amino acid sequence of the CDR L2 contained in SEQ ID NO: 55. In some embodiments, the antibody comprises a CDR L3 having an amino acid sequence of the CDRL3 contained in SEQ ID NO: 55.

In some embodiments, the antibody comprises a CDR H1 and a CDR H2 having amino acid sequences of the CDR H1 and the CDR H2 contained in SEQ ID NO: 27. In some embodiments, the antibody comprises a CDR H1 and a CDR H3 having amino acid sequences of the CDR H1 and the CDR H3 contained in SEQ ID NO: 27. In some embodiments, the antibody comprises a CDR H2 and a CDR H3 having amino acid sequences of the CDR H2 and the CDR H3 contained in SEQ ID NO: 27. In some embodiments, the antibody comprises a CDR L1 and a CDR L2 having amino acid sequences of the CDR L1 and the CDR L2 contained in SEQ ID NO: 55. In some embodiments, the antibody comprises a CDR L1 and a CDR L3 having amino acid sequences of the CDR L1 and the CDR L3 contained in SEQ ID NO: 55. In some embodiments, the antibody comprises a CDR L2 and a CDR L3 having amino acid sequences of the CDR L2 and the CDR L3 contained in SEQ ID NO: 55.

In some embodiments, the antibody comprises a CDR H1 and a CDR L1 having amino acid sequences of the CDR H1 and the CDR L1 contained in SEQ ID NO: 27 and SEQ ID NO: 55 respectively. In some embodiments, the antibody comprises a CDR H1 and a CDR L2 having amino acid sequences of the CDR H1 and the CDR L2 contained in SEQ ID NO: 27 and SEQ ID NO: 55 respectively. In some embodiments, the antibody comprises a CDR H1 and a CDR L3 having amino acid sequences of the CDR H1 and the CDR L3 contained in SEQ ID NO: 27 and SEQ ID NO: 55 respectively. In some embodiments, the antibody comprises a CDR H2 and a CDR L1 having amino acid sequences of the CDR H2 and the CDR L1 contained in SEQ ID NO: 27 and SEQ ID NO: 55 respectively. In some embodiments, the antibody comprises a CDR H2 and a CDR L2 having amino acid sequences of the CDR H2 and the CDR L2 contained in SEQ ID NO: 27 and SEQ ID NO: 55 respectively. In some embodiments, the antibody comprises a CDR H2 and a CDR L3 having amino acid sequences of the CDR H2 and the CDR L3 contained in SEQ ID NO: 27 and SEQ ID NO: 55 respectively. In some embodiments, the antibody comprises a CDR H3 and a CDR L1 having amino acid sequences of the CDR H3 and the CDR L1 contained in SEQ ID NO: 27 and SEQ ID NO: 55 respectively. In some embodiments, the antibody comprises a CDR H3 and a CDR L2 having amino acid sequences of the CDR H3 and the CDR L2 contained in SEQ ID NO: 27 and SEQ ID NO: 55 respectively. In some embodiments, the antibody comprises a CDR H3 and a CDR L3 having amino acid sequences of the CDR H3 and the CDR L3 contained in SEQ ID NO: 27 and SEQ ID NO: 55 respectively.

In some embodiments, the antibody comprises a CDR H1, a CDR H2, and a CDR H3 having amino acid sequences of the CDR H1, the CDR H2, and the CDR H3 contained in SEQ ID NO: 27. In some embodiments, the antibody comprises a CDR L1, a CDR L2, and a CDR L3 having amino acid sequences of the CDR L1, the CDR L2, and the CDR L3 contained in SEQ ID NO: 55.

In some embodiments, the antibody comprises a CDR H1, a CDR H2, and a CDR L1 having amino acid sequences of the CDR H1, the CDR H2, and the CDR L1 contained in SEQ ID NO: 27 and SEQ ID NO: 55 respectively. In some embodiments, the antibody comprises a CDR H1, a CDR H2, and a CDR L2 having amino acid sequences of the CDR H1, the CDR H2, and the CDR L2 contained in SEQ ID NO: 27 and SEQ ID NO: 55 respectively. In some embodiments, the antibody comprises a CDR H1, a CDR H2, and a CDR L3 having amino acid sequences of the CDR H1, the CDR H2, and the CDR L3 contained in SEQ ID NO: 27 and SEQ ID NO: 55 respectively.

In some embodiments, the antibody comprises a CDR H1, a CDR H3, and a CDR L1 having amino acid sequences of the CDR H1, the CDR H3, and the CDR L1 contained in SEQ ID NO: 27 and SEQ ID NO: 55 respectively. In some embodiments, the antibody comprises a CDR H1, a CDR H3, and a CDR L2 having amino acid sequences of the CDR H1, the CDR H3, and the CDR L2 contained in SEQ ID NO: 27 and SEQ ID NO: 55 respectively. In some embodiments, the antibody comprises a CDR H1, a CDR H3, and a CDR L3 having amino acid sequences of the CDR H1, the CDR H3, and the CDR L3 contained in SEQ ID NO: 27 and SEQ ID NO: 55 respectively.

In some embodiments, the antibody comprises a CDR H2, a CDR H3, and a CDR L1 having amino acid sequences of the CDR H2, the CDR H3, and the CDR L1 contained in SEQ ID NO: 27 and SEQ ID NO: 55 respectively. In some embodiments, the antibody comprises a CDR H2, a CDR H3, and a CDR L2 having amino acid sequences of the CDR H2, the CDR H3, and the CDR L2 contained in SEQ ID NO: 27 and SEQ ID NO: 55 respectively. In some embodiments, the antibody comprises a CDR H2, a CDR H3, and a CDR L3 having amino acid sequences of the CDR H2, the CDR H3, and the CDR L3 contained in SEQ ID NO: 27 and SEQ ID NO: 55 respectively.

In some embodiments, the antibody comprises a CDR H1, a CDR L1, and a CDR L2 having amino acid sequences of the CDR H1, the CDR L1, and the CDR L2 contained in SEQ ID NO: 27 and SEQ ID NO: 55 respectively. In some embodiments, the antibody comprises a CDR H1, a CDR L1, and a CDR L3 having amino acid sequences of the CDR H1, the CDR L1, and the CDR L3 contained in SEQ ID NO: 27 and SEQ ID NO: 55 respectively. In some embodiments, the antibody comprises a CDR H1, a CDR L2, and a CDR L3 having amino acid sequences of the CDR H1, the CDR L2, and the CDR L3 contained in SEQ ID NO: 27 and SEQ ID NO: 55 respectively.

In some embodiments, the antibody comprises a CDR H2, a CDR L1, and a CDR L2 having amino acid sequences of the CDR H2, the CDR L1, and the CDR L2 contained in SEQ ID NO: 27 and SEQ ID NO: 55 respectively. In some embodiments, the antibody comprises a CDR H2, a CDR L1, and a CDR L3 having amino acid sequences of the CDR H2, the CDR L1, and the CDR L3 contained in SEQ ID NO: 27 and SEQ ID NO: 55 respectively. In some embodiments, the antibody comprises a CDR H2, a CDR L2, and a CDR L3 having amino acid sequences of the CDR H2, the CDR L2, and the CDR L3 contained in SEQ ID NO: 27 and SEQ ID NO: 55 respectively.

In some embodiments, the antibody comprises a CDR H3, a CDR L1, and a CDR L2 having amino acid sequences of the CDR H3, the CDR L1, and the CDR L2 contained in SEQ ID NO: 27 and SEQ ID NO: 55 respectively. In some embodiments, the antibody comprises a CDR H3, a CDR L1, and a CDR L3 having amino acid sequences of the CDR H3, the CDR L1, and the CDR L3 contained in SEQ ID NO: 27 and SEQ ID NO: 55 respectively. In some embodiments, the antibody comprises a CDR H3, a CDR L2, and a CDR L3 having amino acid sequences of the CDR H3, the CDR L2, and the CDR L3 contained in SEQ ID NO: 27 and SEQ ID NO: 55 respectively.

In some embodiments, the antibody comprises a CDR H3, a CDR Ll, a CDR L2 and a CDR L3 having amino acid sequences of the CDR H3, the CDR L1, the CDR L2 and the CDR L3 contained in SEQ ID NO: 27 and SEQ ID NO: 55 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 27 and SEQ ID NO: 55 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 27 and SEQ ID NO: 55 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 27 and SEQ ID NO: 55 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 27 and SEQ ID NO: 55 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 27 and SEQ ID NO: 55 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 27 and SEQ ID NO: 55 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 27 and SEQ ID NO: 55 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 27 and SEQ ID NO: 55 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 27 and SEQ ID NO: 55 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 27 and SEQ ID NO: 55 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 27 and SEQ ID NO: 55 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 27 and SEQ ID NO: 55 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 27 and SEQ ID NO: 55 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 27 and SEQ ID NO: 55 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 27 and SEQ ID NO: 55 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 27 and SEQ ID NO: 55 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 27 and SEQ ID NO: 55 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 27 and SEQ ID NO: 55 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 27 and SEQ ID NO: 55 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 27 and SEQ ID NO: 55 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 27 and SEQ ID NO: 55 respectively.

The residues from each of these CDR regions are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 71. In some embodiments, the antibody comprises a CDR H2 of SEQ ID NO: 69. In some embodiments, the antibody comprises a CDR H3 of SEQ ID NO: 72. In some embodiments, the antibody comprises a CDR L1 of SEQ ID NO: 86. In some embodiments, the antibody comprises a CDR L2 of SEQ ID NO: 87. In other embodiments, the antibody comprises a CDR L3 of SEQ ID NO: 88.

In some embodiments, the antibody comprises a CDR H1 of SEQ ID NO: 71 and a CDR H2 of SEQ ID NO: 69. In some embodiments, the antibody comprises a CDR H1 of SEQ ID NO: 71 and a CDR H3 of SEQ ID NO: 72. In some embodiments, the antibody comprises a CDR H2 of SEQ ID NO: 69 and a CDR H3 of SEQ ID NO: 72. In some embodiments, the antibody comprises a CDR L1 of SEQ ID NO: 86 and a CDR L2 of SEQ ID NO: 87. In some embodiments, the antibody comprises a CDR L1 of SEQ ID NO: 86 and a CDR L3 of SEQ ID NO: 88. In some embodiments, the antibody comprises a CDR L2 of SEQ ID NO: 87 and a CDR L3 of SEQ ID NO: 88.

In some embodiments, the antibody comprises a CDR H1 of SEQ ID NO: 71 and a CDR L1 of SEQ ID NO: 86. In some embodiments, the antibody comprises a CDR H1 of SEQ ID NO: 71 and a CDR L2 of SEQ ID NO: 87. In some embodiments, the antibody comprises a CDR H1 of SEQ ID NO: 71 and a CDR L3 of SEQ ID NO: 88. In some embodiments, the antibody comprises a CDR H2 of SEQ ID NO: 69 and a CDR L1 of SEQ ID NO: 86. In some embodiments, the antibody comprises a CDR H2 of SEQ ID NO: 69 and a CDR L2 of SEQ ID NO: 87. In some embodiments, the Ab comprises a CDR H2 of SEQ ID NO: 69 and a CDRL3 of SEQ ID NO: 88. In some embodiments, the antibody comprises a CDR H3 of SEQ ID NO: 72 and a CDR L1 of SEQ ID NO: 86. In some embodiments, the antibody comprises a CDR H3 of SEQ ID NO: 72 and a CDR L2 of SEQ ID NO: 87. In some embodiments, the antibody comprises a CDR H3 of SEQ ID NO: 72 and a CDR L3 of SEQ ID NO: 88.

In some embodiments, the antibody comprises a CDR H1 of SEQ ID NO: 71, a CDR H2 of SEQ ID NO: 69, and a CDR H3 of SEQ ID NO: 72. In some embodiments, the antibody comprises a CDR L1 of SEQ ID NO: 86, a CDR L2 of SEQ ID NO: 87, and a CDR L3 of SEQ ID NO: 88.

In some embodiments, the antibody comprises a CDR H1 of SEQ ID NO: 71, a CDR H2 of SEQ ID NO: 69, and a CDR L1 of SEQ ID NO: 86. In some embodiments, the antibody comprises a CDR H1 of SEQ ID NO: 71, a CDR H2 of SEQ ID NO: 69, and a CDR L2 of SEQ ID NO: 87. In some embodiments, the antibody comprises a CDR H1 of SEQ ID NO: 71, a CDR H2 of SEQ ID NO: 69, and a CDR L3 of SEQ ID NO: 88. In some embodiments, the antibody comprises a CDR H1 of SEQ ID NO: 71, a CDR H3 of SEQ ID NO: 72, and a CDR L1 of SEQ ID NO: 86. In some embodiments, the antibody comprises a CDR H1 of SEQ ID NO: 71, a CDR H3 of SEQ ID NO: 72, and a CDR L2 of SEQ ID NO: 87. In some embodiments, the antibody comprises a CDR H1 of SEQ ID NO: 71, a CDR H3 of SEQ ID NO: 72, and a CDR L3 of SEQ ID NO: 88. In some embodiments, the antibody comprises a CDR H2 of SEQ ID NO: 69, a CDR H3 of SEQ ID NO: 72, and a CDR L1 of SEQ ID NO: 86. In some embodiments, the antibody comprises a CDR H2 of SEQ ID NO: 69, a CDR H3 of SEQ ID NO: 72, and a CDR L2 of SEQ ID NO: 87. In some embodiments, the antibody comprises a CDR H2 of SEQ ID NO: 69, a CDR H3 of SEQ ID NO: 72, and a CDR L3 of SEQ ID NO: 88.

In some embodiments, the antibody comprises a CDR H1 of SEQ ID NO: 71, a CDR L1 of SEQ ID NO: 86, and a CDR L2 of SEQ ID NO: 87. In some embodiments, the antibody comprises a CDR H1 of SEQ ID NO: 71, a CDR L1 of SEQ ID NO: 86, and a CDR L3 of SEQ ID NO: 88. In some embodiments, the antibody comprises a CDR H1 of SEQ ID NO: 71, a CDR L2 of SEQ ID NO: 87, and a CDR L3 of SEQ ID NO: 88. In some embodiments, the antibody comprises a CDR H2 of SEQ ID NO: 69, a CDR L1 of SEQ ID NO: 86, and a CDR L2 of SEQ ID NO: 87. In some embodiments, the antibody comprises a CDR H2 of SEQ ID NO: 69, a CDR L1 of SEQ ID NO: 86, and a CDR L3 of SEQ ID NO: 88. In some embodiments, the antibody comprises a CDR H2 of SEQ ID NO: 69, a CDR L2 of SEQ ID NO: 87, and a CDR L3 of SEQ ID NO: 88. In some embodiments, the antibody comprises a CDR H3 of SEQ ID NO: 72, a CDR L1 of SEQ ID NO: 86, and a CDR L2 of SEQ ID NO: 87. In some embodiments, the antibody comprises a CDR H3 of SEQ ID NO: 72, a CDR L1 of SEQ ID NO: 86, and a CDR L3 of SEQ ID NO: 88. In some embodiments, the antibody comprises a CDR H3 of SEQ ID NO: 72, a CDR L2 of SEQ ID NO: 87, and a CDR L3 of SEQ ID NO: 88.

In some embodiments, the antibody comprises a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H2 of SEQ ID NO: 69, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 69, a CDR H3 of SEQ ID NO: 72, and a CDR L3 of SEQ ID NO: 88. In some embodiments, the antibody comprises a CDR H1 of SEQ ID NO: 71, a CDR H2 of SEQ ID NO: 69, a CDR H3 of SEQ ID NO: 72, and a CDR L2 of SEQ ID NO: 87. In some embodiments, the antibody comprises a CDR H1 of SEQ ID NO: 71, a CDR H2 of SEQ ID NO: 69, a CDR H3 of SEQ ID NO: 72, and a CDR L1 of SEQ ID NO: 86.

In some embodiments, the antibody comprises a CDR H2 of SEQ ID NO: 69, a CDR H3 of SEQ ID NO: 72, a CDR L2 of SEQ ID NO: 87, and a CDR L3 of SEQ ID NO: 88. In some embodiments, the antibody comprises a CDR H2 of SEQ ID NO: 69, a CDR H3 of SEQ ID NO: 72, a CDR L1 of SEQ ID NO: 86, and a CDR L3 of SEQ ID NO: 88. In some embodiments, the antibody comprises a CDR H2 of SEQ ID NO: 69, a CDR H3 of SEQ ID NO: 72, a CDR L1 of SEQ ID NO: 86, and a CDR L2 of SEQ ID NO: 87. In some embodiments, the antibody comprises a CDR H1 of SEQ ID NO: 71, a CDR H3 of SEQ ID NO: 72, a CDR L2 of SEQ ID NO: 87, and a CDR L3 of SEQ ID NO: 88. In some embodiments, the antibody comprises a CDR H1 of SEQ ID NO: 71, a CDR H3 of SEQ ID NO: 72, a CDR L1 of SEQ ID NO: 86, and a CDR L3 of SEQ ID NO: 88. In some embodiments, the antibody comprises a CDR H1 of SEQ ID NO: 71, a CDR H3 of SEQ ID NO: 72, a CDR L1 of SEQ ID NO: 86, and a CDR L2 of SEQ ID NO: 87. In some embodiments, the antibody comprises a CDR H1 of SEQ ID NO: 71, a CDR H2 of SEQ ID NO: 69, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 69, a CDR L1 of SEQ ID NO: 86, and a CDR L3 of SEQ ID NO: 88. In some embodiments, the antibody comprises a CDR H1 of SEQ ID NO: 71, a CDR H2 of SEQ ID NO: 69, a CDR L1 of SEQ ID NO: 86, and a CDR L2 of SEQ ID NO: 87.

In some embodiments, the antibody comprises a CDR H1 of SEQ ID NO: 71, a CDR H2 of SEQ ID NO: 69, a CDR H3 of SEQ ID NO: 72, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 69, a CDR H3 of SEQ ID NO: 72, a CDR L1 of SEQ ID NO: 86, and a CDR L3 of SEQ ID NO: 88. In some embodiments, the antibody comprises a CDR H1 of SEQ ID NO: 71, a CDR H2 of SEQ ID NO: 69, a CDR H3 of SEQ ID NO: 72, a CDR L1 of SEQ ID NO: 86, and a CDR L2 of SEQ ID NO: 87. In some embodiments, the antibody comprises a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 69, a CDR L1 of SEQ ID NO: 86, a CDR L2 of SEQ ID NO: 87, and a CDR L3 of SEQ ID NO: 88.

In a specific embodiment, the antibody comprises a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88.

In some embodiments, the antibody provided herein comprises one or more CDR regions from antibody 144L124B or antibody 144L180A.

In some embodiments, the antibody comprises a CDR H1 having an amino acid sequence of the CDR H1 contained in SEQ ID NO: 31. In some embodiments, the antibody comprises a CDR H2 having an amino acid sequence of the CDR H2 contained in SEQ ID NO: 31. In some embodiments, the antibody comprises a CDR H3 having an amino acid sequence of the CDR H3 contained in SEQ ID NO: 31. In some embodiments, the antibody comprises a CDR L1 having an amino acid sequence of the CDR L1 contained in SEQ ID NO: 55. In some embodiments, the antibody comprises a CDR L2 having an amino acid sequence of the CDR L2 contained in SEQ ID NO: 55. In some embodiments, the antibody comprises a CDR L3 having an amino acid sequence of the CDRL3 contained in SEQ ID NO: 55.

In some embodiments, the antibody comprises a CDR H1 and a CDR H2 having amino acid sequences of the CDR H1 and the CDR H2 contained in SEQ ID NO: 31. In some embodiments, the antibody comprises a CDR H1 and a CDR H3 having amino acid sequences of the CDR H1 and the CDR H3 contained in SEQ ID NO: 31. In some embodiments, the antibody comprises a CDR H2 and a CDR H3 having amino acid sequences of the CDR H2 and the CDR H3 contained in SEQ ID NO: 31. In some embodiments, the antibody comprises a CDR L1 and a CDR L2 having amino acid sequences of the CDR L1 and the CDR L2 contained in SEQ ID NO: 55. In some embodiments, the antibody comprises a CDR L1 and a CDR L3 having amino acid sequences of the CDR L1 and the CDR L3 contained in SEQ ID NO: 55. In some embodiments, the antibody comprises a CDR L2 and a CDR L3 having amino acid sequences of the CDR L2 and the CDR L3 contained in SEQ ID NO: 55.

In some embodiments, the antibody comprises a CDR H1 and a CDR L1 having amino acid sequences of the CDR H1 and the CDR L1 contained in SEQ ID NO: 31 and SEQ ID NO: 55 respectively. In some embodiments, the antibody comprises a CDR H1 and a CDR L2 having amino acid sequences of the CDR H1 and the CDR L2 contained in SEQ ID NO: 31 and SEQ ID NO: 55 respectively. In some embodiments, the antibody comprises a CDR H1 and a CDR L3 having amino acid sequences of the CDR H1 and the CDR L3 contained in SEQ ID NO: 31 and SEQ ID NO: 55 respectively. In some embodiments, the antibody comprises a CDR H2 and a CDR L1 having amino acid sequences of the CDR H2 and the CDR L1 contained in SEQ ID NO: 31 and SEQ ID NO: 55 respectively. In some embodiments, the antibody comprises a CDR H2 and a CDR L2 having amino acid sequences of the CDR H2 and the CDR L2 contained in SEQ ID NO: 31 and SEQ ID NO: 55 respectively. In some embodiments, the antibody comprises a CDR H2 and a CDR L3 having amino acid sequences of the CDR H2 and the CDR L3 contained in SEQ ID NO: 31 and SEQ ID NO: 55 respectively. In some embodiments, the antibody comprises a CDR H3 and a CDR L1 having amino acid sequences of the CDR H3 and the CDR L1 contained in SEQ ID NO: 31 and SEQ ID NO: 55 respectively. In some embodiments, the antibody comprises a CDR H3 and a CDR L2 having amino acid sequences of the CDR H3 and the CDR L2 contained in SEQ ID NO: 31 and SEQ ID NO: 55 respectively. In some embodiments, the antibody comprises a CDR H3 and a CDR L3 having amino acid sequences of the CDR H3 and the CDR L3 contained in SEQ ID NO: 31 and SEQ ID NO: 55 respectively.

In some embodiments, the antibody comprises a CDR H1, a CDR H2, and a CDR H3 having amino acid sequences of the CDR H1, the CDR H2, and the CDR H3 contained in SEQ ID NO: 31. In some embodiments, the antibody comprises a CDR L1, a CDR L2, and a CDR L3 having amino acid sequences of the CDR L1, the CDR L2, and the CDR L3 contained in SEQ ID NO: 55.

In some embodiments, the antibody comprises a CDR H1, a CDR H2, and a CDR L1 having amino acid sequences of the CDR H1, the CDR H2, and the CDR L1 contained in SEQ ID NO: 31 and SEQ ID NO: 55 respectively. In some embodiments, the antibody comprises a CDR H1, a CDR H2, and a CDR L2 having amino acid sequences of the CDR H1, the CDR H2, and the CDR L2 contained in SEQ ID NO: 31 and SEQ ID NO: 55 respectively. In some embodiments, the antibody comprises a CDR H1, a CDR H2, and a CDR L3 having amino acid sequences of the CDR H1, the CDR H2, and the CDR L3 contained in SEQ ID NO: 31 and SEQ ID NO: 55 respectively.

In some embodiments, the antibody comprises a CDR H1, a CDR H3, and a CDR L1 having amino acid sequences of the CDR H1, the CDR H3, and the CDR L1 contained in SEQ ID NO: 31 and SEQ ID NO: 55 respectively. In some embodiments, the antibody comprises a CDR H1, a CDR H3, and a CDR L2 having amino acid sequences of the CDR H1, the CDR H3, and the CDR L2 contained in SEQ ID NO: 31 and SEQ ID NO: 55 respectively. In some embodiments, the antibody comprises a CDR H1, a CDR H3, and a CDR L3 having amino acid sequences of the CDR H1, the CDR H3, and the CDR L3 contained in SEQ ID NO: 31 and SEQ ID NO: 55 respectively.

In some embodiments, the antibody comprises a CDR H2, a CDR H3, and a CDR L1 having amino acid sequences of the CDR H2, the CDR H3, and the CDR L1 contained in SEQ ID NO: 31 and SEQ ID NO: 55 respectively. In some embodiments, the antibody comprises a CDR H2, a CDR H3, and a CDR L2 having amino acid sequences of the CDR H2, the CDR H3, and the CDR L2 contained in SEQ ID NO: 31 and SEQ ID NO: 55 respectively. In some embodiments, the antibody comprises a CDR H2, a CDR H3, and a CDR L3 having amino acid sequences of the CDR H2, the CDR H3, and the CDR L3 contained in SEQ ID NO: 31 and SEQ ID NO: 55 respectively.

In some embodiments, the antibody comprises a CDR H1, a CDR L1, and a CDR L2 having amino acid sequences of the CDR H1, the CDR L1, and the CDR L2 contained in SEQ ID NO: 31 and SEQ ID NO: 55 respectively. In some embodiments, the antibody comprises a CDR H1, a CDR L1, and a CDR L3 having amino acid sequences of the CDR H1, the CDR L1, and the CDR L3 contained in SEQ ID NO: 31 and SEQ ID NO: 55 respectively. In some embodiments, the antibody comprises a CDR H1, a CDR L2, and a CDR L3 having amino acid sequences of the CDR H1, the CDR L2, and the CDR L3 contained in SEQ ID NO: 31 and SEQ ID NO: 55 respectively.

In some embodiments, the antibody comprises a CDR H2, a CDR L1, and a CDR L2 having amino acid sequences of the CDR H2, the CDR L1, and the CDR L2 contained in SEQ ID NO: 31 and SEQ ID NO: 55 respectively. In some embodiments, the antibody comprises a CDR H2, a CDR L1, and a CDR L3 having amino acid sequences of the CDR H2, the CDR L1, and the CDR L3 contained in SEQ ID NO: 31 and SEQ ID NO: 55 respectively. In some embodiments, the antibody comprises a CDR H2, a CDR L2, and a CDR L3 having amino acid sequences of the CDR H2, the CDR L2, and the CDR L3 contained in SEQ ID NO: 31 and SEQ ID NO: 55 respectively.

In some embodiments, the antibody comprises a CDR H3, a CDR L1, and a CDR L2 having amino acid sequences of the CDR H3, the CDR L1, and the CDR L2 contained in SEQ ID NO: 31 and SEQ ID NO: 55 respectively. In some embodiments, the antibody comprises a CDR H3, a CDR L1, and a CDR L3 having amino acid sequences of the CDR H3, the CDR L1, and the CDR L3 contained in SEQ ID NO: 31 and SEQ ID NO: 55 respectively. In some embodiments, the antibody comprises a CDR H3, a CDR L2, and a CDR L3 having amino acid sequences of the CDR H3, the CDR L2, and the CDR L3 contained in SEQ ID NO: 31 and SEQ ID NO: 55 respectively.

In some embodiments, the antibody comprises a CDR H3, a CDR L1, a CDR L2 and a CDR L3 having amino acid sequences of the CDR H3, the CDR L1, the CDR L2 and the CDR L3 contained in SEQ ID NO: 31 and SEQ ID NO: 55 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 31 and SEQ ID NO: 55 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 31 and SEQ ID NO: 55 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 31 and SEQ ID NO: 55 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 31 and SEQ ID NO: 55 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 31 and SEQ ID NO: 55 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 31 and SEQ ID NO: 55 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 31 and SEQ ID NO: 55 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 31 and SEQ ID NO: 55 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 31 and SEQ ID NO: 55 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 31 and SEQ ID NO: 55 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 31 and SEQ ID NO: 55 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 31 and SEQ ID NO: 55 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 31 and SEQ ID NO: 55 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 31 and SEQ ID NO: 55 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 31 and SEQ ID NO: 55 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 31 and SEQ ID NO: 55 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 31 and SEQ ID NO: 55 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 31 and SEQ ID NO: 55 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 31 and SEQ ID NO: 55 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 31 and SEQ ID NO: 55 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 31 and SEQ ID NO: 55 respectively.

The residues from each of CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 71. In some embodiments, the antibody comprises a CDR H2 of SEQ ID NO: 73. In some embodiments, the antibody comprises a CDR H3 of SEQ ID NO: 74. In some embodiments, the antibody comprises a CDR L1 of SEQ ID NO: 86. In some embodiments, the antibody comprises a CDR L2 of SEQ ID NO: 87. In other embodiments, the antibody comprises a CDR L3 of SEQ ID NO: 88.

In some embodiments, the antibody comprises a CDR H1 of SEQ ID NO: 71 and a CDR H2 of SEQ ID NO: 73. In some embodiments, the antibody comprises a CDR H1 of SEQ ID NO: 71 and a CDR H3 of SEQ ID NO: 74. In some embodiments, the antibody comprises a CDR H2 of SEQ ID NO: 73 and a CDR H3 of SEQ ID NO: 74. In some embodiments, the antibody comprises a CDR L1 of SEQ ID NO: 86 and a CDR L2 of SEQ ID NO: 87. In some embodiments, the antibody comprises a CDR L1 of SEQ ID NO: 86 and a CDR L3 of SEQ ID NO: 88. In some embodiments, the antibody comprises a CDR L2 of SEQ ID NO: 87 and a CDR L3 of SEQ ID NO: 88.

In some embodiments, the antibody comprises a CDR H1 of SEQ ID NO: 71 and a CDR L1 of SEQ ID NO: 86. In some embodiments, the antibody comprises a CDR H1 of SEQ ID NO: 71 and a CDR L2 of SEQ ID NO: 87. In some embodiments, the antibody comprises a CDR H1 of SEQ ID NO: 71 and a CDR L3 of SEQ ID NO: 88. In some embodiments, the antibody comprises a CDR H2 of SEQ ID NO: 73 and a CDR L1 of SEQ ID NO: 86. In some embodiments, the antibody comprises a CDR H2 of SEQ ID NO: 73 and a CDR L2 of SEQ ID NO: 87. In some embodiments, the Ab comprises a CDR H2 of SEQ ID NO: 73 and a CDRL3 of SEQ ID NO: 88. In some embodiments, the antibody comprises a CDR H3 of SEQ ID NO: 74 and a CDR L1 of SEQ ID NO: 86. In some embodiments, the antibody comprises a CDR H3 of SEQ ID NO: 74 and a CDR L2 of SEQ ID NO: 87. In some embodiments, the antibody comprises a CDR H3 of SEQ ID NO: 74 and a CDR L3 of SEQ ID NO: 88.

In some embodiments, the antibody comprises a CDR H1 of SEQ ID NO: 71, a CDR H2 of SEQ ID NO: 73, and a CDR H3 of SEQ ID NO: 74. In some embodiments, the antibody comprises a CDR L1 of SEQ ID NO: 86, a CDR L2 of SEQ ID NO: 87, and a CDR L3 of SEQ ID NO: 88.

In some embodiments, the antibody comprises a CDR H1 of SEQ ID NO: 71, a CDR H2 of SEQ ID NO: 73, and a CDR L1 of SEQ ID NO: 86. In some embodiments, the antibody comprises a CDR H1 of SEQ ID NO: 71, a CDR H2 of SEQ ID NO: 73, and a CDR L2 of SEQ ID NO: 87. In some embodiments, the antibody comprises a CDR H1 of SEQ ID NO: 71, a CDR H2 of SEQ ID NO: 73, and a CDR L3 of SEQ ID NO: 88. In some embodiments, the antibody comprises a CDR H1 of SEQ ID NO: 71, a CDR H3 of SEQ ID NO: 74, and a CDR L1 of SEQ ID NO: 86. In some embodiments, the antibody comprises a CDR H1 of SEQ ID NO: 71, a CDR H3 of SEQ ID NO: 74, and a CDR L2 of SEQ ID NO: 87. In some embodiments, the antibody comprises a CDR H1 of SEQ ID NO: 71, a CDR H3 of SEQ ID NO: 74, and a CDR L3 of SEQ ID NO: 88. In some embodiments, the antibody comprises a CDR H2 of SEQ ID NO: 73, a CDR H3 of SEQ ID NO: 74, and a CDR L1 of SEQ ID NO: 86. In some embodiments, the antibody comprises a CDR H2 of SEQ ID NO: 73, a CDR H3 of SEQ ID NO: 74, and a CDR L2 of SEQ ID NO: 87. In some embodiments, the antibody comprises a CDR H2 of SEQ ID NO: 73, a CDR H3 of SEQ ID NO: 74, and a CDR L3 of SEQ ID NO: 88.

In some embodiments, the antibody comprises a CDR H1 of SEQ ID NO: 71, a CDR L1 of SEQ ID NO: 86, and a CDR L2 of SEQ ID NO: 87. In some embodiments, the antibody comprises a CDR H1 of SEQ ID NO: 71, a CDR L1 of SEQ ID NO: 86, and a CDR L3 of SEQ ID NO: 88. In some embodiments, the antibody comprises a CDR H1 of SEQ ID NO: 71, a CDR L2 of SEQ ID NO: 87, and a CDR L3 of SEQ ID NO: 88. In some embodiments, the antibody comprises a CDR H2 of SEQ ID NO: 73, a CDR L1 of SEQ ID NO: 86, and a CDR L2 of SEQ ID NO: 87. In some embodiments, the antibody comprises a CDR H2 of SEQ ID NO: 73, a CDR L1 of SEQ ID NO: 86, and a CDR L3 of SEQ ID NO: 88. In some embodiments, the antibody comprises a CDR H2 of SEQ ID NO: 73, a CDR L2 of SEQ ID NO: 87, and a CDR L3 of SEQ ID NO: 88. In some embodiments, the antibody comprises a CDR H3 of SEQ ID NO: 74, a CDR L1 of SEQ ID NO: 86, and a CDR L2 of SEQ ID NO: 87. In some embodiments, the antibody comprises a CDR H3 of SEQ ID NO: 74, a CDR L1 of SEQ ID NO: 86, and a CDR L3 of SEQ ID NO: 88. In some embodiments, the antibody comprises a CDR H3 of SEQ ID NO: 74, a CDR L2 of SEQ ID NO: 87, and a CDR L3 of SEQ ID NO: 88.

In some embodiments, the antibody comprises a CDR H3 of SEQ ID NO: 74, a CDR L1 of SEQ ID NO: 86, a CDR L2 of SEQ ID NO: 87, and a CDR L3 of SEQ ID NO: 88. In some embodiments, the antibody comprises a CDR H2 of SEQ ID NO: 73, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 73, a CDR H3 of SEQ ID NO: 74, and a CDR L3 of SEQ ID NO: 88. In some embodiments, the antibody comprises a CDR H1 of SEQ ID NO: 71, a CDR H2 of SEQ ID NO: 73, a CDR H3 of SEQ ID NO: 74, and a CDR L2 of SEQ ID NO: 87. In some embodiments, the antibody comprises a CDR H1 of SEQ ID NO: 71, a CDR H2 of SEQ ID NO: 73, a CDR H3 of SEQ ID NO: 74, and a CDR L1 of SEQ ID NO: 86.

In some embodiments, the antibody comprises a CDR H2 of SEQ ID NO: 73, a CDR H3 of SEQ ID NO: 74, a CDR L2 of SEQ ID NO: 87, and a CDR L3 of SEQ ID NO: 88. In some embodiments, the antibody comprises a CDR H2 of SEQ ID NO: 73, a CDR H3 of SEQ ID NO: 74, a CDR L1 of SEQ ID NO: 86, and a CDR L3 of SEQ ID NO: 88. In some embodiments, the antibody comprises a CDR H2 of SEQ ID NO: 73, a CDR H3 of SEQ ID NO: 74, a CDR L1 of SEQ ID NO: 86, and a CDR L2 of SEQ ID NO: 87. In some embodiments, the antibody comprises a CDR H1 of SEQ ID NO: 71, a CDR H3 of SEQ ID NO: 74, a CDR L2 of SEQ ID NO: 87, and a CDR L3 of SEQ ID NO: 88. In some embodiments, the antibody comprises a CDR H1 of SEQ ID NO: 71, a CDR H3 of SEQ ID NO: 74, a CDR L1 of SEQ ID NO: 86, and a CDR L3 of SEQ ID NO: 88. In some embodiments, the antibody comprises a CDR H1 of SEQ ID NO: 71, a CDR H3 of SEQ ID NO: 74, a CDR L1 of SEQ ID NO: 86, and a CDR L2 of SEQ ID NO: 87. In some embodiments, the antibody comprises a CDR H1 of SEQ ID NO: 71, a CDR H2 of SEQ ID NO: 73, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 73, a CDR L1 of SEQ ID NO: 86, and a CDR L3 of SEQ ID NO: 88. In some embodiments, the antibody comprises a CDR H1 of SEQ ID NO: 71, a CDR H2 of SEQ ID NO: 73, a CDR L1 of SEQ ID NO: 86, and a CDR L2 of SEQ ID NO: 87.

In some embodiments, the antibody comprises a CDR H1 of SEQ ID NO: 71, a CDR H2 of SEQ ID NO: 73, a CDR H3 of SEQ ID NO: 74, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 73, a CDR H3 of SEQ ID NO: 74, a CDR L1 of SEQ ID NO: 86, and a CDR L3 of SEQ ID NO: 88. In some embodiments, the antibody comprises a CDR H1 of SEQ ID NO: 71, a CDR H2 of SEQ ID NO: 73, a CDR H3 of SEQ ID NO: 74, a CDR L1 of SEQ ID NO: 86, and a CDR L2 of SEQ ID NO: 87. In some embodiments, the antibody comprises a CDR H2 of SEQ ID NO: 73, a CDR H3 of SEQ ID NO: 74, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H3 of SEQ ID NO: 74, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 71, a CDR H2 of SEQ ID NO: 73, a CDR L1 of SEQ ID NO: 86, a CDR L2 of SEQ ID NO: 87, and a CDR L3 of SEQ ID NO: 88.

In a specific embodiment, the antibody comprises a CDR H1 of SEQ ID NO: 71, a CDR H2 of SEQ ID NO: 73, a CDR H3 of SEQ ID NO: 74, a CDR L1 of SEQ ID NO: 86, a CDR L2 of SEQ ID NO: 87, and a CDR L3 of SEQ ID NO: 88.

In some embodiments, the antibody provided herein comprises one or more CDR regions from antibody 144L133B.

In some embodiments, the antibody comprises a CDR H1 having an amino acid sequence of the CDR H1 contained in SEQ ID NO: 35. In some embodiments, the antibody comprises a CDR H2 having an amino acid sequence of the CDR H2 contained in SEQ ID NO: 35. In some embodiments, the antibody comprises a CDR H3 having an amino acid sequence of the CDR H3 contained in SEQ ID NO: 35. In some embodiments, the antibody comprises a CDR L1 having an amino acid sequence of the CDR L1 contained in SEQ ID NO: 55. In some embodiments, the antibody comprises a CDR L2 having an amino acid sequence of the CDR L2 contained in SEQ ID NO: 55. In some embodiments, the antibody comprises a CDR L3 having an amino acid sequence of the CDRL3 contained in SEQ ID NO: 55.

In some embodiments, the antibody comprises a CDR H1 and a CDR H2 having amino acid sequences of the CDR H1 and the CDR H2 contained in SEQ ID NO: 35. In some embodiments, the antibody comprises a CDR H1 and a CDR H3 having amino acid sequences of the CDR H1 and the CDR H3 contained in SEQ ID NO: 35. In some embodiments, the antibody comprises a CDR H2 and a CDR H3 having amino acid sequences of the CDR H2 and the CDR H3 contained in SEQ ID NO: 35. In some embodiments, the antibody comprises a CDR L1 and a CDR L2 having amino acid sequences of the CDR L1 and the CDR L2 contained in SEQ ID NO: 55. In some embodiments, the antibody comprises a CDR L1 and a CDR L3 having amino acid sequences of the CDR L1 and the CDR L3 contained in SEQ ID NO: 55. In some embodiments, the antibody comprises a CDR L2 and a CDR L3 having amino acid sequences of the CDR L2 and the CDR L3 contained in SEQ ID NO: 55.

In some embodiments, the antibody comprises a CDR H1 and a CDR L1 having amino acid sequences of the CDR H1 and the CDR L1 contained in SEQ ID NO: 35 and SEQ ID NO: 55 respectively. In some embodiments, the antibody comprises a CDR H1 and a CDR L2 having amino acid sequences of the CDR H1 and the CDR L2 contained in SEQ ID NO: 35 and SEQ ID NO: 55 respectively. In some embodiments, the antibody comprises a CDR H1 and a CDR L3 having amino acid sequences of the CDR H1 and the CDR L3 contained in SEQ ID NO: 35 and SEQ ID NO: 55 respectively. In some embodiments, the antibody comprises a CDR H2 and a CDR L1 having amino acid sequences of the CDR H2 and the CDR L1 contained in SEQ ID NO: 35 and SEQ ID NO: 55 respectively. In some embodiments, the antibody comprises a CDR H2 and a CDR L2 having amino acid sequences of the CDR H2 and the CDR L2 contained in SEQ ID NO: 35 and SEQ ID NO: 55 respectively. In some embodiments, the antibody comprises a CDR H2 and a CDR L3 having amino acid sequences of the CDR H2 and the CDR L3 contained in SEQ ID NO: 35 and SEQ ID NO: 55 respectively. In some embodiments, the antibody comprises a CDR H3 and a CDR L1 having amino acid sequences of the CDR H3 and the CDR L1 contained in SEQ ID NO: 35 and SEQ ID NO: 55 respectively. In some embodiments, the antibody comprises a CDR H3 and a CDR L2 having amino acid sequences of the CDR H3 and the CDR L2 contained in SEQ ID NO: 35 and SEQ ID NO: 55 respectively. In some embodiments, the antibody comprises a CDR H3 and a CDR L3 having amino acid sequences of the CDR H3 and the CDR L3 contained in SEQ ID NO: 35 and SEQ ID NO: 55 respectively.

In some embodiments, the antibody comprises a CDR H1, a CDR H2, and a CDR H3 having amino acid sequences of the CDR H1, the CDR H2, and the CDR H3 contained in SEQ ID NO: 35. In some embodiments, the antibody comprises a CDR L1, a CDR L2, and a CDR L3 having amino acid sequences of the CDR L1, the CDR L2, and the CDR L3 contained in SEQ ID NO: 55.

In some embodiments, the antibody comprises a CDR H1, a CDR H2, and a CDR L1 having amino acid sequences of the CDR H1, the CDR H2, and the CDR L1 contained in SEQ ID NO: 35 and SEQ ID NO: 55 respectively. In some embodiments, the antibody comprises a CDR H1, a CDR H2, and a CDR L2 having amino acid sequences of the CDR H1, the CDR H2, and the CDR L2 contained in SEQ ID NO: 35 and SEQ ID NO: 55 respectively. In some embodiments, the antibody comprises a CDR H1, a CDR H2, and a CDR L3 having amino acid sequences of the CDR H1, the CDR H2, and the CDR L3 contained in SEQ ID NO: 35 and SEQ ID NO: 55 respectively.

In some embodiments, the antibody comprises a CDR H1, a CDR H3, and a CDR L1 having amino acid sequences of the CDR H1, the CDR H3, and the CDR L1 contained in SEQ ID NO: 35 and SEQ ID NO: 55 respectively. In some embodiments, the antibody comprises a CDR H1, a CDR H3, and a CDR L2 having amino acid sequences of the CDR H1, the CDR H3, and the CDR L2 contained in SEQ ID NO: 35 and SEQ ID NO: 55 respectively. In some embodiments, the antibody comprises a CDR H1, a CDR H3, and a CDR L3 having amino acid sequences of the CDR H1, the CDR H3, and the CDR L3 contained in SEQ ID NO: 35 and SEQ ID NO: 55 respectively.

In some embodiments, the antibody comprises a CDR H2, a CDR H3, and a CDR L1 having amino acid sequences of the CDR H2, the CDR H3, and the CDR L1 contained in SEQ ID NO: 35 and SEQ ID NO: 55 respectively. In some embodiments, the antibody comprises a CDR H2, a CDR H3, and a CDR L2 having amino acid sequences of the CDR H2, the CDR H3, and the CDR L2 contained in SEQ ID NO: 35 and SEQ ID NO: 55 respectively. In some embodiments, the antibody comprises a CDR H2, a CDR H3, and a CDR L3 having amino acid sequences of the CDR H2, the CDR H3, and the CDR L3 contained in SEQ ID NO: 35 and SEQ ID NO: 55 respectively.

In some embodiments, the antibody comprises a CDR H1, a CDR L1, and a CDR L2 having amino acid sequences of the CDR H1, the CDR L1, and the CDR L2 contained in SEQ ID NO: 35 and SEQ ID NO: 55 respectively. In some embodiments, the antibody comprises a CDR H1, a CDR L1, and a CDR L3 having amino acid sequences of the CDR H1, the CDR L1, and the CDR L3 contained in SEQ ID NO: 35 and SEQ ID NO: 55 respectively. In some embodiments, the antibody comprises a CDR H1, a CDR L2, and a CDR L3 having amino acid sequences of the CDR H1, the CDR L2, and the CDR L3 contained in SEQ ID NO: 35 and SEQ ID NO: 55 respectively.

In some embodiments, the antibody comprises a CDR H2, a CDR L1, and a CDR L2 having amino acid sequences of the CDR H2, the CDR L1, and the CDR L2 contained in SEQ ID NO: 35 and SEQ ID NO: 55 respectively. In some embodiments, the antibody comprises a CDR H2, a CDR L1, and a CDR L3 having amino acid sequences of the CDR H2, the CDR L1, and the CDR L3 contained in SEQ ID NO: 35 and SEQ ID NO: 55 respectively. In some embodiments, the antibody comprises a CDR H2, a CDR L2, and a CDR L3 having amino acid sequences of the CDR H2, the CDR L2, and the CDR L3 contained in SEQ ID NO: 35 and SEQ ID NO: 55 respectively.

In some embodiments, the antibody comprises a CDR H3, a CDR L1, and a CDR L2 having amino acid sequences of the CDR H3, the CDR L1, and the CDR L2 contained in SEQ ID NO: 35 and SEQ ID NO: 55 respectively. In some embodiments, the antibody comprises a CDR H3, a CDR L1, and a CDR L3 having amino acid sequences of the CDR H3, the CDR L1, and the CDR L3 contained in SEQ ID NO: 35 and SEQ ID NO: 55 respectively. In some embodiments, the antibody comprises a CDR H3, a CDR L2, and a CDR L3 having amino acid sequences of the CDR H3, the CDR L2, and the CDR L3 contained in SEQ ID NO: 35 and SEQ ID NO: 55 respectively.

In some embodiments, the antibody comprises a CDR H3, a CDR L1, a CDR L2 and a CDR L3 having amino acid sequences of the CDR H3, the CDR L1, the CDR L2 and the CDR L3 contained in SEQ ID NO: 35 and SEQ ID NO: 55 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 35 and SEQ ID NO: 55 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 35 and SEQ ID NO: 55 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 35 and SEQ ID NO: 55 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 35 and SEQ ID NO: 55 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 35 and SEQ ID NO: 55 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 35 and SEQ ID NO: 55 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 35 and SEQ ID NO: 55 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 35 and SEQ ID NO: 55 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 35 and SEQ ID NO: 55 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 35 and SEQ ID NO: 55 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 35 and SEQ ID NO: 55 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 35 and SEQ ID NO: 55 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 35 and SEQ ID NO: 55 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 35 and SEQ ID NO: 55 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 35 and SEQ ID NO: 55 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 35 and SEQ ID NO: 55 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 35 and SEQ ID NO: 55 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 35 and SEQ ID NO: 55 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 35 and SEQ ID NO: 55 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 35 and SEQ ID NO: 55 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 35 and SEQ ID NO: 55 respectively.

The residues from each of these CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 75. In some embodiments, the antibody comprises a CDR H2 of SEQ ID NO: 69. In some embodiments, the antibody comprises a CDR H3 of SEQ ID NO: 72. In some embodiments, the antibody comprises a CDR L1 of SEQ ID NO: 86. In some embodiments, the antibody comprises a CDR L2 of SEQ ID NO: 87. In other embodiments, the antibody comprises a CDR L3 of SEQ ID NO: 88.

In some embodiments, the antibody comprises a CDR H1 of SEQ ID NO: 75 and a CDR H2 of SEQ ID NO: 69. In some embodiments, the antibody comprises a CDR H1 of SEQ ID NO: 75 and a CDR H3 of SEQ ID NO: 72. In some embodiments, the antibody comprises a CDR H2 of SEQ ID NO: 69 and a CDR H3 of SEQ ID NO: 72. In some embodiments, the antibody comprises a CDR L1 of SEQ ID NO: 86 and a CDR L2 of SEQ ID NO: 87. In some embodiments, the antibody comprises a CDR L1 of SEQ ID NO: 86 and a CDR L3 of SEQ ID NO: 88. In some embodiments, the antibody comprises a CDR L2 of SEQ ID NO: 87 and a CDR L3 of SEQ ID NO: 88.

In some embodiments, the antibody comprises a CDR H1 of SEQ ID NO: 75 and a CDR L1 of SEQ ID NO: 86. In some embodiments, the antibody comprises a CDR H1 of SEQ ID NO: 75 and a CDR L2 of SEQ ID NO: 87. In some embodiments, the antibody comprises a CDR H1 of SEQ ID NO: 75 and a CDR L3 of SEQ ID NO: 88. In some embodiments, the antibody comprises a CDR H2 of SEQ ID NO: 69 and a CDR L1 of SEQ ID NO: 86. In some embodiments, the antibody comprises a CDR H2 of SEQ ID NO: 69 and a CDR L2 of SEQ ID NO: 87. In some embodiments, the Ab comprises a CDR H2 of SEQ ID NO: 69 and a CDRL3 of SEQ ID NO: 88. In some embodiments, the antibody comprises a CDR H3 of SEQ ID NO: 72 and a CDR L1 of SEQ ID NO: 86. In some embodiments, the antibody comprises a CDR H3 of SEQ ID NO: 72 and a CDR L2 of SEQ ID NO: 87. In some embodiments, the antibody comprises a CDR H3 of SEQ ID NO: 72 and a CDR L3 of SEQ ID NO: 88.

In some embodiments, the antibody comprises a CDR H1 of SEQ ID NO: 75, a CDR H2 of SEQ ID NO: 69, and a CDR H3 of SEQ ID NO: 72. In some embodiments, the antibody comprises a CDR L1 of SEQ ID NO: 86, a CDR L2 of SEQ ID NO: 87, and a CDR L3 of SEQ ID NO: 88.

In some embodiments, the antibody comprises a CDR H1 of SEQ ID NO: 75, a CDR H2 of SEQ ID NO: 69, and a CDR L1 of SEQ ID NO: 86. In some embodiments, the antibody comprises a CDR H1 of SEQ ID NO: 75, a CDR H2 of SEQ ID NO: 69, and a CDR L2 of SEQ ID NO: 87. In some embodiments, the antibody comprises a CDR H1 of SEQ ID NO: 75, a CDR H2 of SEQ ID NO: 69, and a CDR L3 of SEQ ID NO: 88. In some embodiments, the antibody comprises a CDR H1 of SEQ ID NO: 75, a CDR H3 of SEQ ID NO: 72, and a CDR L1 of SEQ ID NO: 86. In some embodiments, the antibody comprises a CDR H1 of SEQ ID NO: 75, a CDR H3 of SEQ ID NO: 72, and a CDR L2 of SEQ ID NO: 87. In some embodiments, the antibody comprises a CDR H1 of SEQ ID NO: 75, a CDR H3 of SEQ ID NO: 72, and a CDR L3 of SEQ ID NO: 88. In some embodiments, the antibody comprises a CDR H2 of SEQ ID NO: 69, a CDR H3 of SEQ ID NO: 72, and a CDR L1 of SEQ ID NO: 86. In some embodiments, the antibody comprises a CDR H2 of SEQ ID NO: 69, a CDR H3 of SEQ ID NO: 72, and a CDR L2 of SEQ ID NO: 87. In some embodiments, the antibody comprises a CDR H2 of SEQ ID NO: 69, a CDR H3 of SEQ ID NO: 72, and a CDR L3 of SEQ ID NO: 88.

In some embodiments, the antibody comprises a CDR H1 of SEQ ID NO: 75, a CDR L1 of SEQ ID NO: 86, and a CDR L2 of SEQ ID NO: 87. In some embodiments, the antibody comprises a CDR H1 of SEQ ID NO: 75, a CDR L1 of SEQ ID NO: 86, and a CDR L3 of SEQ ID NO: 88. In some embodiments, the antibody comprises a CDR H1 of SEQ ID NO: 75, a CDR L2 of SEQ ID NO: 87, and a CDR L3 of SEQ ID NO: 88. In some embodiments, the antibody comprises a CDR H2 of SEQ ID NO: 69, a CDR L1 of SEQ ID NO: 86, and a CDR L2 of SEQ ID NO: 87. In some embodiments, the antibody comprises a CDR H2 of SEQ ID NO: 69, a CDR L1 of SEQ ID NO: 86, and a CDR L3 of SEQ ID NO: 88. In some embodiments, the antibody comprises a CDR H2 of SEQ ID NO: 69, a CDR L2 of SEQ ID NO: 87, and a CDR L3 of SEQ ID NO: 88. In some embodiments, the antibody comprises a CDR H3 of SEQ ID NO: 72, a CDR L1 of SEQ ID NO: 86, and a CDR L2 of SEQ ID NO: 87. In some embodiments, the antibody comprises a CDR H3 of SEQ ID NO: 72, a CDR L1 of SEQ ID NO: 86, and a CDR L3 of SEQ ID NO: 88. In some embodiments, the antibody comprises a CDR H3 of SEQ ID NO: 72, a CDR L2 of SEQ ID NO: 87, and a CDR L3 of SEQ ID NO: 88.

In some embodiments, the antibody comprises a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H2 of SEQ ID NO: 69, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 75, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 75, a CDR H2 of SEQ ID NO: 69, a CDR H3 of SEQ ID NO: 72, and a CDR L3 of SEQ ID NO: 88. In some embodiments, the antibody comprises a CDR H1 of SEQ ID NO: 75, a CDR H2 of SEQ ID NO: 69, a CDR H3 of SEQ ID NO: 72, and a CDR L2 of SEQ ID NO: 87. In some embodiments, the antibody comprises a CDR H1 of SEQ ID NO: 75, a CDR H2 of SEQ ID NO: 69, a CDR H3 of SEQ ID NO: 72, and a CDR L1 of SEQ ID NO: 86.

In some embodiments, the antibody comprises a CDR H2 of SEQ ID NO: 69, a CDR H3 of SEQ ID NO: 72, a CDR L2 of SEQ ID NO: 87, and a CDR L3 of SEQ ID NO: 88. In some embodiments, the antibody comprises a CDR H2 of SEQ ID NO: 69, a CDR H3 of SEQ ID NO: 72, a CDR L1 of SEQ ID NO: 86, and a CDR L3 of SEQ ID NO: 88. In some embodiments, the antibody comprises a CDR H2 of SEQ ID NO: 69, a CDR H3 of SEQ ID NO: 72, a CDR L1 of SEQ ID NO: 86, and a CDR L2 of SEQ ID NO: 87. In some embodiments, the antibody comprises a CDR H1 of SEQ ID NO: 75, a CDR H3 of SEQ ID NO: 72, a CDR L2 of SEQ ID NO: 87, and a CDR L3 of SEQ ID NO: 88. In some embodiments, the antibody comprises a CDR H1 of SEQ ID NO: 75, a CDR H3 of SEQ ID NO: 72, a CDR L1 of SEQ ID NO: 86, and a CDR L3 of SEQ ID NO: 88. In some embodiments, the antibody comprises a CDR H1 of SEQ ID NO: 75, a CDR H3 of SEQ ID NO: 72, a CDR L1 of SEQ ID NO: 86, and a CDR L2 of SEQ ID NO: 87. In some embodiments, the antibody comprises a CDR H1 of SEQ ID NO: 75, a CDR H2 of SEQ ID NO: 69, a CDR L2 of SEQ ID NO: 87, and a CDR L3 of SEQ ID NO: 88. In some embodiments, the antibody comprises a CDR H1 of SEQ ID NO: 75, a CDR H2 of SEQ ID NO: 69, a CDR L1 of SEQ ID NO: 86, and a CDR L3 of SEQ ID NO: 88. In some embodiments, the antibody comprises a CDR H1 of SEQ ID NO: 75, a CDR H2 of SEQ ID NO: 69, a CDR L1 of SEQ ID NO: 86, and a CDR L2 of SEQ ID NO: 87.

In some embodiments, the antibody comprises a CDR H1 of SEQ ID NO: 75, a CDR H2 of SEQ ID NO: 69, a CDR H3 of SEQ ID NO: 72, a CDR L2 of SEQ ID NO: 87, and a CDR L3 of SEQ ID NO: 88. In some embodiments, the antibody comprises a CDR H1 of SEQ ID NO: 75, a CDR H2 of SEQ ID NO: 69, a CDR H3 of SEQ ID NO: 72, a CDR L1 of SEQ ID NO: 86, and a CDR L3 of SEQ ID NO: 88. In some embodiments, the antibody comprises a CDR H1 of SEQ ID NO: 75, a CDR H2 of SEQ ID NO: 69, a CDR H3 of SEQ ID NO: 72, a CDR L1 of SEQ ID NO: 86, and a CDR L2 of SEQ ID NO: 87. In some embodiments, the antibody comprises a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 75, a CDR H3 of SEQ ID NO: 72, a CDR L1 of SEQ ID NO: 86, a CDR L2 of SEQ ID NO: 87, and a CDR L3 of SEQ ID NO: 88. In some embodiments, the antibody comprises a CDR H1 of SEQ ID NO: 75, a CDR H2 of SEQ ID NO: 69, a CDR L1 of SEQ ID NO: 86, a CDR L2 of SEQ ID NO: 87, and a CDR L3 of SEQ ID NO: 88.

In a specific embodiment, the antibody comprises a CDR H1 of SEQ ID NO: 75, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88.

In some embodiments, the antibody provided herein comprises one or more CDR regions from antibody 144L472A.

In some embodiments, the antibody comprises a CDR H1 having an amino acid sequence of the CDR H1 contained in SEQ ID NO: 39. In some embodiments, the antibody comprises a CDR H2 having an amino acid sequence of the CDR H2 contained in SEQ ID NO: 39. In some embodiments, the antibody comprises a CDR H3 having an amino acid sequence of the CDR H3 contained in SEQ ID NO: 39. In some embodiments, the antibody comprises a CDR L1 having an amino acid sequence of the CDR L1 contained in SEQ ID NO: 59. In some embodiments, the antibody comprises a CDR L2 having an amino acid sequence of the CDR L2 contained in SEQ ID NO: 59. In some embodiments, the antibody comprises a CDR L3 having an amino acid sequence of the CDRL3 contained in SEQ ID NO: 59.

In some embodiments, the antibody comprises a CDR H1 and a CDR H2 having amino acid sequences of the CDR H1 and the CDR H2 contained in SEQ ID NO: 39. In some embodiments, the antibody comprises a CDR H1 and a CDR H3 having amino acid sequences of the CDR H1 and the CDR H3 contained in SEQ ID NO: 39. In some embodiments, the antibody comprises a CDR H2 and a CDR H3 having amino acid sequences of the CDR H2 and the CDR H3 contained in SEQ ID NO: 39. In some embodiments, the antibody comprises a CDR L1 and a CDR L2 having amino acid sequences of the CDR L1 and the CDR L2 contained in SEQ ID NO: 59. In some embodiments, the antibody comprises a CDR L1 and a CDR L3 having amino acid sequences of the CDR L1 and the CDR L3 contained in SEQ ID NO: 59. In some embodiments, the antibody comprises a CDR L2 and a CDR L3 having amino acid sequences of the CDR L2 and the CDR L3 contained in SEQ ID NO: 59.

In some embodiments, the antibody comprises a CDR H1 and a CDR L1 having amino acid sequences of the CDR H1 and the CDR L1 contained in SEQ ID NO: 39 and SEQ ID NO: 59 respectively. In some embodiments, the antibody comprises a CDR H1 and a CDR L2 having amino acid sequences of the CDR H1 and the CDR L2 contained in SEQ ID NO: 39 and SEQ ID NO: 59 respectively. In some embodiments, the antibody comprises a CDR H1 and a CDR L3 having amino acid sequences of the CDR H1 and the CDR L3 contained in SEQ ID NO: 39 and SEQ ID NO: 59 respectively. In some embodiments, the antibody comprises a CDR H2 and a CDR L1 having amino acid sequences of the CDR H2 and the CDR L1 contained in SEQ ID NO: 39 and SEQ ID NO: 59 respectively. In some embodiments, the antibody comprises a CDR H2 and a CDR L2 having amino acid sequences of the CDR H2 and the CDR L2 contained in SEQ ID NO: 39 and SEQ ID NO: 59 respectively. In some embodiments, the antibody comprises a CDR H2 and a CDR L3 having amino acid sequences of the CDR H2 and the CDR L3 contained in SEQ ID NO: 39 and SEQ ID NO: 59 respectively. In some embodiments, the antibody comprises a CDR H3 and a CDR L1 having amino acid sequences of the CDR H3 and the CDR L1 contained in SEQ ID NO: 39 and SEQ ID NO: 59 respectively. In some embodiments, the antibody comprises a CDR H3 and a CDR L2 having amino acid sequences of the CDR H3 and the CDR L2 contained in SEQ ID NO: 39 and SEQ ID NO: 59 respectively. In some embodiments, the antibody comprises a CDR H3 and a CDR L3 having amino acid sequences of the CDR H3 and the CDR L3 contained in SEQ ID NO: 39 and SEQ ID NO: 59 respectively.

In some embodiments, the antibody comprises a CDR H1, a CDR H2, and a CDR H3 having amino acid sequences of the CDR H1, the CDR H2, and the CDR H3 contained in SEQ ID NO: 39. In some embodiments, the antibody comprises a CDR L1, a CDR L2, and a CDR L3 having amino acid sequences of the CDR L1, the CDR L2, and the CDR L3 contained in SEQ ID NO: 59.

In some embodiments, the antibody comprises a CDR H1, a CDR H2, and a CDR L1 having amino acid sequences of the CDR H1, the CDR H2, and the CDR L1 contained in SEQ ID NO: 39 and SEQ ID NO: 59 respectively. In some embodiments, the antibody comprises a CDR H1, a CDR H2, and a CDR L2 having amino acid sequences of the CDR H1, the CDR H2, and the CDR L2 contained in SEQ ID NO: 39 and SEQ ID NO: 59 respectively. In some embodiments, the antibody comprises a CDR H1, a CDR H2, and a CDR L3 having amino acid sequences of the CDR H1, the CDR H2, and the CDR L3 contained in SEQ ID NO: 39 and SEQ ID NO: 59 respectively.

In some embodiments, the antibody comprises a CDR H1, a CDR H3, and a CDR L1 having amino acid sequences of the CDR H1, the CDR H3, and the CDR L1 contained in SEQ ID NO: 39 and SEQ ID NO: 59 respectively. In some embodiments, the antibody comprises a CDR H1, a CDR H3, and a CDR L2 having amino acid sequences of the CDR H1, the CDR H3, and the CDR L2 contained in SEQ ID NO: 39 and SEQ ID NO: 59 respectively. In some embodiments, the antibody comprises a CDR H1, a CDR H3, and a CDR L3 having amino acid sequences of the CDR H1, the CDR H3, and the CDR L3 contained in SEQ ID NO: 39 and SEQ ID NO: 59 respectively.

In some embodiments, the antibody comprises a CDR H2, a CDR H3, and a CDR L1 having amino acid sequences of the CDR H2, the CDR H3, and the CDR L1 contained in SEQ ID NO: 39 and SEQ ID NO: 59 respectively. In some embodiments, the antibody comprises a CDR H2, a CDR H3, and a CDR L2 having amino acid sequences of the CDR H2, the CDR H3, and the CDR L2 contained in SEQ ID NO: 39 and SEQ ID NO: 59 respectively. In some embodiments, the antibody comprises a CDR H2, a CDR H3, and a CDR L3 having amino acid sequences of the CDR H2, the CDR H3, and the CDR L3 contained in SEQ ID NO: 39 and SEQ ID NO: 59 respectively.

In some embodiments, the antibody comprises a CDR H1, a CDR L1, and a CDR L2 having amino acid sequences of the CDR H1, the CDR L1, and the CDR L2 contained in SEQ ID NO: 39 and SEQ ID NO: 59 respectively. In some embodiments, the antibody comprises a CDR H1, a CDR L1, and a CDR L3 having amino acid sequences of the CDR H1, the CDR L1, and the CDR L3 contained in SEQ ID NO: 39 and SEQ ID NO: 59 respectively. In some embodiments, the antibody comprises a CDR H1, a CDR L2, and a CDR L3 having amino acid sequences of the CDR H1, the CDR L2, and the CDR L3 contained in SEQ ID NO: 39 and SEQ ID NO: 59 respectively.

In some embodiments, the antibody comprises a CDR H2, a CDR L1, and a CDR L2 having amino acid sequences of the CDR H2, the CDR L1, and the CDR L2 contained in SEQ ID NO: 39 and SEQ ID NO: 59 respectively. In some embodiments, the antibody comprises a CDR H2, a CDR L1, and a CDR L3 having amino acid sequences of the CDR H2, the CDR L1, and the CDR L3 contained in SEQ ID NO: 39 and SEQ ID NO: 59 respectively. In some embodiments, the antibody comprises a CDR H2, a CDR L2, and a CDR L3 having amino acid sequences of the CDR H2, the CDR L2, and the CDR L3 contained in SEQ ID NO: 39 and SEQ ID NO: 59 respectively.

In some embodiments, the antibody comprises a CDR H3, a CDR L1, and a CDR L2 having amino acid sequences of the CDR H3, the CDR L1, and the CDR L2 contained in SEQ ID NO: 39 and SEQ ID NO: 59 respectively. In some embodiments, the antibody comprises a CDR H3, a CDR L1, and a CDR L3 having amino acid sequences of the CDR H3, the CDR L1, and the CDR L3 contained in SEQ ID NO: 39 and SEQ ID NO: 59 respectively. In some embodiments, the antibody comprises a CDR H3, a CDR L2, and a CDR L3 having amino acid sequences of the CDR H3, the CDR L2, and the CDR L3 contained in SEQ ID NO: 39 and SEQ ID NO: 59 respectively.

In some embodiments, the antibody comprises a CDR H3, a CDR L1, a CDR L2 and a CDR L3 having amino acid sequences of the CDR H3, the CDR L1, the CDR L2 and the CDR L3 contained in SEQ ID NO: 39 and SEQ ID NO: 59 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 39 and SEQ ID NO: 59 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 39 and SEQ ID NO: 59 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 39 and SEQ ID NO: 59 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 39 and SEQ ID NO: 59 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 39 and SEQ ID NO: 59 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 39 and SEQ ID NO: 59 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 39 and SEQ ID NO: 59 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 39 and SEQ ID NO: 59 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 39 and SEQ ID NO: 59 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 39 and SEQ ID NO: 59 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 39 and SEQ ID NO: 59 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 39 and SEQ ID NO: 59 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 39 and SEQ ID NO: 59 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 39 and SEQ ID NO: 59 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 39 and SEQ ID NO: 59 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 39 and SEQ ID NO: 59 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 39 and SEQ ID NO: 59 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 39 and SEQ ID NO: 59 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 39 and SEQ ID NO: 59 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 39 and SEQ ID NO: 59 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 39 and SEQ ID NO: 59 respectively.

The residues from each of CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 75. In some embodiments, the antibody comprises a CDR H2 of SEQ ID NO: 76. In some embodiments, the antibody comprises a CDR H3 of SEQ ID NO: 77. In some embodiments, the antibody comprises a CDR L1 of SEQ ID NO: 83. In some embodiments, the antibody comprises a CDR L2 of SEQ ID NO: 87. In other embodiments, the antibody comprises a CDR L3 of SEQ ID NO: 89.

In some embodiments, the antibody comprises a CDR H1 of SEQ ID NO: 75 and a CDR H2 of SEQ ID NO: 76. In some embodiments, the antibody comprises a CDR H1 of SEQ ID NO: 75 and a CDR H3 of SEQ ID NO: 77. In some embodiments, the antibody comprises a CDR H2 of SEQ ID NO: 76 and a CDR H3 of SEQ ID NO: 77. In some embodiments, the antibody comprises a CDR L1 of SEQ ID NO: 83 and a CDR L2 of SEQ ID NO: 87. In some embodiments, the antibody comprises a CDR L1 of SEQ ID NO: 83 and a CDR L3 of SEQ ID NO: 89. In some embodiments, the antibody comprises a CDR L2 of SEQ ID NO: 87 and a CDR L3 of SEQ ID NO: 89.

In some embodiments, the antibody comprises a CDR H1 of SEQ ID NO: 75 and a CDR L1 of SEQ ID NO: 83. In some embodiments, the antibody comprises a CDR H1 of SEQ ID NO: 75 and a CDR L2 of SEQ ID NO: 87. In some embodiments, the antibody comprises a CDR H1 of SEQ ID NO: 75 and a CDR L3 of SEQ ID NO: 89. In some embodiments, the antibody comprises a CDR H2 of SEQ ID NO: 76 and a CDR L1 of SEQ ID NO: 83. In some embodiments, the antibody comprises a CDR H2 of SEQ ID NO: 76 and a CDR L2 of SEQ ID NO: 87. In some embodiments, the Ab comprises a CDR H2 of SEQ ID NO: 76 and a CDRL3 of SEQ ID NO: 89. In some embodiments, the antibody comprises a CDR H3 of SEQ ID NO: 77 and a CDR L1 of SEQ ID NO: 83. In some embodiments, the antibody comprises a CDR H3 of SEQ ID NO: 77 and a CDR L2 of SEQ ID NO: 87. In some embodiments, the antibody comprises a CDR H3 of SEQ ID NO: 77 and a CDR L3 of SEQ ID NO: 89.

In some embodiments, the antibody comprises a CDR H1 of SEQ ID NO: 75, a CDR H2 of SEQ ID NO: 76, and a CDR H3 of SEQ ID NO: 77. In some embodiments, the antibody comprises a CDR L1 of SEQ ID NO: 83, a CDR L2 of SEQ ID NO: 87, and a CDR L3 of SEQ ID NO: 89.

In some embodiments, the antibody comprises a CDR H1 of SEQ ID NO: 75, a CDR H2 of SEQ ID NO: 76, and a CDR L1 of SEQ ID NO: 83. In some embodiments, the antibody comprises a CDR H1 of SEQ ID NO: 75, a CDR H2 of SEQ ID NO: 76, and a CDR L2 of SEQ ID NO: 87. In some embodiments, the antibody comprises a CDR H1 of SEQ ID NO: 75, a CDR H2 of SEQ ID NO: 76, and a CDR L3 of SEQ ID NO: 89. In some embodiments, the antibody comprises a CDR H1 of SEQ ID NO: 75, a CDR H3 of SEQ ID NO: 77, and a CDR L1 of SEQ ID NO: 83. In some embodiments, the antibody comprises a CDR H1 of SEQ ID NO: 75, a CDR H3 of SEQ ID NO: 77, and a CDR L2 of SEQ ID NO: 87. In some embodiments, the antibody comprises a CDR H1 of SEQ ID NO: 75, a CDR H3 of SEQ ID NO: 77, and a CDR L3 of SEQ ID NO: 89. In some embodiments, the antibody comprises a CDR H2 of SEQ ID NO: 76, a CDR H3 of SEQ ID NO: 77, and a CDR L1 of SEQ ID NO: 83. In some embodiments, the antibody comprises a CDR H2 of SEQ ID NO: 76, a CDR H3 of SEQ ID NO: 77, and a CDR L2 of SEQ ID NO: 87. In some embodiments, the antibody comprises a CDR H2 of SEQ ID NO: 76, a CDR H3 of SEQ ID NO: 77, and a CDR L3 of SEQ ID NO: 89.

In some embodiments, the antibody comprises a CDR H1 of SEQ ID NO: 75, a CDR L1 of SEQ ID NO: 83, and a CDR L2 of SEQ ID NO: 87. In some embodiments, the antibody comprises a CDR H1 of SEQ ID NO: 75, a CDR L1 of SEQ ID NO: 83, and a CDR L3 of SEQ ID NO: 89. In some embodiments, the antibody comprises a CDR H1 of SEQ ID NO: 75, a CDR L2 of SEQ ID NO: 87, and a CDR L3 of SEQ ID NO: 89. In some embodiments, the antibody comprises a CDR H2 of SEQ ID NO: 76, a CDR L1 of SEQ ID NO: 83, and a CDR L2 of SEQ ID NO: 87. In some embodiments, the antibody comprises a CDR H2 of SEQ ID NO: 76, a CDR L1 of SEQ ID NO: 83, and a CDR L3 of SEQ ID NO: 89. In some embodiments, the antibody comprises a CDR H2 of SEQ ID NO: 76, a CDR L2 of SEQ ID NO: 87, and a CDR L3 of SEQ ID NO: 89. In some embodiments, the antibody comprises a CDR H3 of SEQ ID NO: 77, a CDR L1 of SEQ ID NO: 83, and a CDR L2 of SEQ ID NO: 87. In some embodiments, the antibody comprises a CDR H3 of SEQ ID NO: 77, a CDR L1 of SEQ ID NO: 83, and a CDR L3 of SEQ ID NO: 89. In some embodiments, the antibody comprises a CDR H3 of SEQ ID NO: 77, a CDR L2 of SEQ ID NO: 87, and a CDR L3 of SEQ ID NO: 89.

In some embodiments, the antibody comprises a CDR H3 of SEQ ID NO: 77, a CDR L1 of SEQ ID NO: 83, a CDR L2 of SEQ ID NO: 87, and a CDR L3 of SEQ ID NO: 89. In some embodiments, the antibody comprises a CDR H2 of SEQ ID NO: 76, a CDR L1 of SEQ ID NO: 83, a CDR L2 of SEQ ID NO: 87, and a CDR L3 of SEQ ID NO: 89. In some embodiments, the antibody comprises a CDR H1 of SEQ ID NO: 75, a CDR L1 of SEQ ID NO: 83, a CDR L2 of SEQ ID NO: 87, and a CDR L3 of SEQ ID NO: 89. In some embodiments, the antibody comprises a CDR H1 of SEQ ID NO: 75, a CDR H2 of SEQ ID NO: 76, a CDR H3 of SEQ ID NO: 77, and a CDR L3 of SEQ ID NO: 89. In some embodiments, the antibody comprises a CDR H1 of SEQ ID NO: 75, a CDR H2 of SEQ ID NO: 76, a CDR H3 of SEQ ID NO: 77, and a CDR L2 of SEQ ID NO: 87. In some embodiments, the antibody comprises a CDR H1 of SEQ ID NO: 75, a CDR H2 of SEQ ID NO: 76, a CDR H3 of SEQ ID NO: 77, and a CDR L1 of SEQ ID NO: 83.

In some embodiments, the antibody comprises a CDR H2 of SEQ ID NO: 76, a CDR H3 of SEQ ID NO: 77, a CDR L2 of SEQ ID NO: 87, and a CDR L3 of SEQ ID NO: 89. In some embodiments, the antibody comprises a CDR H2 of SEQ ID NO: 76, a CDR H3 of SEQ ID NO: 77, a CDR L1 of SEQ ID NO: 83, and a CDR L3 of SEQ ID NO: 89. In some embodiments, the antibody comprises a CDR H2 of SEQ ID NO: 76, a CDR H3 of SEQ ID NO: 77, a CDR L1 of SEQ ID NO: 83, and a CDR L2 of SEQ ID NO: 87. In some embodiments, the antibody comprises a CDR H1 of SEQ ID NO: 75, a CDR H3 of SEQ ID NO: 77, a CDR L2 of SEQ ID NO: 87, and a CDR L3 of SEQ ID NO: 89. In some embodiments, the antibody comprises a CDR H1 of SEQ ID NO: 75, a CDR H3 of SEQ ID NO: 77, a CDR L1 of SEQ ID NO: 83, and a CDR L3 of SEQ ID NO: 89. In some embodiments, the antibody comprises a CDR H1 of SEQ ID NO: 75, a CDR H3 of SEQ ID NO: 77, a CDR L1 of SEQ ID NO: 83, and a CDR L2 of SEQ ID NO: 87. In some embodiments, the antibody comprises a CDR H1 of SEQ ID NO: 75, a CDR H2 of SEQ ID NO: 76, a CDR L2 of SEQ ID NO: 87, and a CDR L3 of SEQ ID NO: 89. In some embodiments, the antibody comprises a CDR H1 of SEQ ID NO: 75, a CDR H2 of SEQ ID NO: 76, a CDR L1 of SEQ ID NO: 83, and a CDR L3 of SEQ ID NO: 89. In some embodiments, the antibody comprises a CDR H1 of SEQ ID NO: 75, a CDR H2 of SEQ ID NO: 76, a CDR L1 of SEQ ID NO: 83, and a CDR L2 of SEQ ID NO: 87.

In some embodiments, the antibody comprises a CDR H1 of SEQ ID NO: 75, a CDR H2 of SEQ ID NO: 76, a CDR H3 of SEQ ID NO: 77, a CDR L2 of SEQ ID NO: 87, and a CDR L3 of SEQ ID NO: 89. In some embodiments, the antibody comprises a CDR H1 of SEQ ID NO: 75, a CDR H2 of SEQ ID NO: 76, a CDR H3 of SEQ ID NO: 77, a CDR L1 of SEQ ID NO: 83, and a CDR L3 of SEQ ID NO: 89. In some embodiments, the antibody comprises a CDR H1 of SEQ ID NO: 75, a CDR H2 of SEQ ID NO: 76, a CDR H3 of SEQ ID NO: 77, a CDR L1 of SEQ ID NO: 83, and a CDR L2 of SEQ ID NO: 87. In some embodiments, the antibody comprises a CDR H2 of SEQ ID NO: 76, a CDR H3 of SEQ ID NO: 77, a CDR L1 of SEQ ID NO: 83, a CDR L2 of SEQ ID NO: 87, and a CDR L3 of SEQ ID NO: 89. In some embodiments, the antibody comprises a CDR H1 of SEQ ID NO: 75, a CDR H3 of SEQ ID NO: 77, a CDR L1 of SEQ ID NO: 83, a CDR L2 of SEQ ID NO: 87, and a CDR L3 of SEQ ID NO: 89. In some embodiments, the antibody comprises a CDR H1 of SEQ ID NO: 75, a CDR H2 of SEQ ID NO: 76, a CDR L1 of SEQ ID NO: 83, a CDR L2 of SEQ ID NO: 87, and a CDR L3 of SEQ ID NO: 89.

In a specific embodiment, the antibody comprises a CDR H1 of SEQ ID NO: 75, a CDR H2 of SEQ ID NO: 76, a CDR H3 of SEQ ID NO: 77, a CDR L1 of SEQ ID NO: 83, a CDR L2 of SEQ ID NO: 87, and a CDR L3 of SEQ ID NO: 89.

In some embodiments, the antibody provided herein comprises one or more CDR regions from antibody 144D666C.

In some embodiments, the antibody comprises a CDR H1 having an amino acid sequence of the CDR H1 contained in SEQ ID NO: 43. In some embodiments, the antibody comprises a CDR H2 having an amino acid sequence of the CDR H2 contained in SEQ ID NO: 43. In some embodiments, the antibody comprises a CDR H3 having an amino acid sequence of the CDR H3 contained in SEQ ID NO: 43. In some embodiments, the antibody comprises a CDR L1 having an amino acid sequence of the CDR L1 contained in SEQ ID NO: 63. In some embodiments, the antibody comprises a CDR L2 having an amino acid sequence of the CDR L2 contained in SEQ ID NO: 63. In some embodiments, the antibody comprises a CDR L3 having an amino acid sequence of the CDRL3 contained in SEQ ID NO: 63.

In some embodiments, the antibody comprises a CDR H1 and a CDR H2 having amino acid sequences of the CDR H1 and the CDR H2 contained in SEQ ID NO: 43. In some embodiments, the antibody comprises a CDR H1 and a CDR H3 having amino acid sequences of the CDR H1 and the CDR H3 contained in SEQ ID NO: 43. In some embodiments, the antibody comprises a CDR H2 and a CDR H3 having amino acid sequences of the CDR H2 and the CDR H3 contained in SEQ ID NO: 43. In some embodiments, the antibody comprises a CDR L1 and a CDR L2 having amino acid sequences of the CDR L1 and the CDR L2 contained in SEQ ID NO: 63. In some embodiments, the antibody comprises a CDR L1 and a CDR L3 having amino acid sequences of the CDR L1 and the CDR L3 contained in SEQ ID NO: 63. In some embodiments, the antibody comprises a CDR L2 and a CDR L3 having amino acid sequences of the CDR L2 and the CDR L3 contained in SEQ ID NO: 63.

In some embodiments, the antibody comprises a CDR H1 and a CDR L1 having amino acid sequences of the CDR H1 and the CDR L1 contained in SEQ ID NO: 43 and SEQ ID NO: 63 respectively. In some embodiments, the antibody comprises a CDR H1 and a CDR L2 having amino acid sequences of the CDR H1 and the CDR L2 contained in SEQ ID NO: 43 and SEQ ID NO: 63 respectively. In some embodiments, the antibody comprises a CDR H1 and a CDR L3 having amino acid sequences of the CDR H1 and the CDR L3 contained in SEQ ID NO: 43 and SEQ ID NO: 63 respectively. In some embodiments, the antibody comprises a CDR H2 and a CDR L1 having amino acid sequences of the CDR H2 and the CDR L1 contained in SEQ ID NO: 43 and SEQ ID NO: 63 respectively. In some embodiments, the antibody comprises a CDR H2 and a CDR L2 having amino acid sequences of the CDR H2 and the CDR L2 contained in SEQ ID NO: 43 and SEQ ID NO: 63 respectively. In some embodiments, the antibody comprises a CDR H2 and a CDR L3 having amino acid sequences of the CDR H2 and the CDR L3 contained in SEQ ID NO: 43 and SEQ ID NO: 63 respectively. In some embodiments, the antibody comprises a CDR H3 and a CDR L1 having amino acid sequences of the CDR H3 and the CDR L1 contained in SEQ ID NO: 43 and SEQ ID NO: 63 respectively. In some embodiments, the antibody comprises a CDR H3 and a CDR L2 having amino acid sequences of the CDR H3 and the CDR L2 contained in SEQ ID NO: 43 and SEQ ID NO: 63 respectively. In some embodiments, the antibody comprises a CDR H3 and a CDR L3 having amino acid sequences of the CDR H3 and the CDR L3 contained in SEQ ID NO: 43 and SEQ ID NO: 63 respectively.

In some embodiments, the antibody comprises a CDR H1, a CDR H2, and a CDR H3 having amino acid sequences of the CDR H1, the CDR H2, and the CDR H3 contained in SEQ ID NO: 43. In some embodiments, the antibody comprises a CDR L1, a CDR L2, and a CDR L3 having amino acid sequences of the CDR L1, the CDR L2, and the CDR L3 contained in SEQ ID NO: 63.

In some embodiments, the antibody comprises a CDR H1, a CDR H2, and a CDR L1 having amino acid sequences of the CDR H1, the CDR H2, and the CDR L1 contained in SEQ ID NO: 43 and SEQ ID NO: 63 respectively. In some embodiments, the antibody comprises a CDR H1, a CDR H2, and a CDR L2 having amino acid sequences of the CDR H1, the CDR H2, and the CDR L2 contained in SEQ ID NO: 43 and SEQ ID NO: 63 respectively. In some embodiments, the antibody comprises a CDR H1, a CDR H2, and a CDR L3 having amino acid sequences of the CDR H1, the CDR H2, and the CDR L3 contained in SEQ ID NO: 43 and SEQ ID NO: 63 respectively.

In some embodiments, the antibody comprises a CDR H1, a CDR H3, and a CDR L1 having amino acid sequences of the CDR H1, the CDR H3, and the CDR L1 contained in SEQ ID NO: 43 and SEQ ID NO: 63 respectively. In some embodiments, the antibody comprises a CDR H1, a CDR H3, and a CDR L2 having amino acid sequences of the CDR H1, the CDR H3, and the CDR L2 contained in SEQ ID NO: 43 and SEQ ID NO: 63 respectively. In some embodiments, the antibody comprises a CDR H1, a CDR H3, and a CDR L3 having amino acid sequences of the CDR H1, the CDR H3, and the CDR L3 contained in SEQ ID NO: 43 and SEQ ID NO: 63 respectively.

In some embodiments, the antibody comprises a CDR H2, a CDR H3, and a CDR L1 having amino acid sequences of the CDR H2, the CDR H3, and the CDR L1 contained in SEQ ID NO: 43 and SEQ ID NO: 63 respectively. In some embodiments, the antibody comprises a CDR H2, a CDR H3, and a CDR L2 having amino acid sequences of the CDR H2, the CDR H3, and the CDR L2 contained in SEQ ID NO: 43 and SEQ ID NO: 63 respectively. In some embodiments, the antibody comprises a CDR H2, a CDR H3, and a CDR L3 having amino acid sequences of the CDR H2, the CDR H3, and the CDR L3 contained in SEQ ID NO: 43 and SEQ ID NO: 63 respectively.

In some embodiments, the antibody comprises a CDR H1, a CDR L1, and a CDR L2 having amino acid sequences of the CDR H1, the CDR L1, and the CDR L2 contained in SEQ ID NO: 43 and SEQ ID NO: 63 respectively. In some embodiments, the antibody comprises a CDR H1, a CDR L1, and a CDR L3 having amino acid sequences of the CDR H1, the CDR L1, and the CDR L3 contained in SEQ ID NO: 43 and SEQ ID NO: 63 respectively. In some embodiments, the antibody comprises a CDR H1, a CDR L2, and a CDR L3 having amino acid sequences of the CDR H1, the CDR L2, and the CDR L3 contained in SEQ ID NO: 43 and SEQ ID NO: 63 respectively.

In some embodiments, the antibody comprises a CDR H2, a CDR L1, and a CDR L2 having amino acid sequences of the CDR H2, the CDR L1, and the CDR L2 contained in SEQ ID NO: 43 and SEQ ID NO: 63 respectively. In some embodiments, the antibody comprises a CDR H2, a CDR L1, and a CDR L3 having amino acid sequences of the CDR H2, the CDR L1, and the CDR L3 contained in SEQ ID NO: 43 and SEQ ID NO: 63 respectively. In some embodiments, the antibody comprises a CDR H2, a CDR L2, and a CDR L3 having amino acid sequences of the CDR H2, the CDR L2, and the CDR L3 contained in SEQ ID NO: 43 and SEQ ID NO: 63 respectively.

In some embodiments, the antibody comprises a CDR H3, a CDR L1, and a CDR L2 having amino acid sequences of the CDR H3, the CDR L1, and the CDR L2 contained in SEQ ID NO: 43 and SEQ ID NO: 63 respectively. In some embodiments, the antibody comprises a CDR H3, a CDR L1, and a CDR L3 having amino acid sequences of the CDR H3, the CDR L1, and the CDR L3 contained in SEQ ID NO: 43 and SEQ ID NO: 63 respectively. In some embodiments, the antibody comprises a CDR H3, a CDR L2, and a CDR L3 having amino acid sequences of the CDR H3, the CDR L2, and the CDR L3 contained in SEQ ID NO: 43 and SEQ ID NO: 63 respectively.

In some embodiments, the antibody comprises a CDR H3, a CDR L1, a CDR L2 and a CDR L3 having amino acid sequences of the CDR H3, the CDR L1, the CDR L2 and the CDR L3 contained in SEQ ID NO: 43 and SEQ ID NO: 63 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 43 and SEQ ID NO: 63 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 43 and SEQ ID NO: 63 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 43 and SEQ ID NO: 63 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 43 and SEQ ID NO: 63 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 43 and SEQ ID NO: 63 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 43 and SEQ ID NO: 63 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 43 and SEQ ID NO: 63 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 43 and SEQ ID NO: 63 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 43 and SEQ ID NO: 63 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 43 and SEQ ID NO: 63 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 43 and SEQ ID NO: 63 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 43 and SEQ ID NO: 63 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 43 and SEQ ID NO: 63 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 43 and SEQ ID NO: 63 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 43 and SEQ ID NO: 63 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 43 and SEQ ID NO: 63 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 43 and SEQ ID NO: 63 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 43 and SEQ ID NO: 63 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 43 and SEQ ID NO: 63 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 43 and SEQ ID NO: 63 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 43 and SEQ ID NO: 63 respectively.

The residues from each of these CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 71. In some embodiments, the antibody comprises a CDR H2 of SEQ ID NO: 78. In some embodiments, the antibody comprises a CDR H3 of SEQ ID NO: 79. In some embodiments, the antibody comprises a CDR L1 of SEQ ID NO: 86. In some embodiments, the antibody comprises a CDR L2 of SEQ ID NO: 90. In other embodiments, the antibody comprises a CDR L3 of SEQ ID NO: 91.

In some embodiments, the antibody comprises a CDR H1 of SEQ ID NO: 71 and a CDR H2 of SEQ ID NO: 78. In some embodiments, the antibody comprises a CDR H1 of SEQ ID NO: 71 and a CDR H3 of SEQ ID NO: 79. In some embodiments, the antibody comprises a CDR H2 of SEQ ID NO: 78 and a CDR H3 of SEQ ID NO: 79. In some embodiments, the antibody comprises a CDR L1 of SEQ ID NO: 86 and a CDR L2 of SEQ ID NO: 90. In some embodiments, the antibody comprises a CDR L1 of SEQ ID NO: 86 and a CDR L3 of SEQ ID NO: 91. In some embodiments, the antibody comprises a CDR L2 of SEQ ID NO: 90 and a CDR L3 of SEQ ID NO: 91.

In some embodiments, the antibody comprises a CDR H1 of SEQ ID NO: 71 and a CDR L1 of SEQ ID NO: 86. In some embodiments, the antibody comprises a CDR H1 of SEQ ID NO: 71 and a CDR L2 of SEQ ID NO: 90. In some embodiments, the antibody comprises a CDR H1 of SEQ ID NO: 71 and a CDR L3 of SEQ ID NO: 91. In some embodiments, the antibody comprises a CDR H2 of SEQ ID NO: 78 and a CDR L1 of SEQ ID NO: 86. In some embodiments, the antibody comprises a CDR H2 of SEQ ID NO: 78 and a CDR L2 of SEQ ID NO: 90. In some embodiments, the Ab comprises a CDR H2 of SEQ ID NO: 78 and a CDRL3 of SEQ ID NO: 91. In some embodiments, the antibody comprises a CDR H3 of SEQ ID NO: 79 and a CDR L1 of SEQ ID NO: 86. In some embodiments, the antibody comprises a CDR H3 of SEQ ID NO: 79 and a CDR L2 of SEQ ID NO: 90. In some embodiments, the antibody comprises a CDR H3 of SEQ ID NO: 79 and a CDR L3 of SEQ ID NO: 91.

In some embodiments, the antibody comprises a CDR H1 of SEQ ID NO: 71, a CDR H2 of SEQ ID NO: 78, and a CDR H3 of SEQ ID NO: 79. In some embodiments, the antibody comprises a CDR L1 of SEQ ID NO: 86, a CDR L2 of SEQ ID NO: 90, and a CDR L3 of SEQ ID NO: 91.

In some embodiments, the antibody comprises a CDR H1 of SEQ ID NO: 71, a CDR H2 of SEQ ID NO: 78, and a CDR L1 of SEQ ID NO: 86. In some embodiments, the antibody comprises a CDR H1 of SEQ ID NO: 71, a CDR H2 of SEQ ID NO: 78, and a CDR L2 of SEQ ID NO: 90. In some embodiments, the antibody comprises a CDR H1 of SEQ ID NO: 71, a CDR H2 of SEQ ID NO: 78, and a CDR L3 of SEQ ID NO: 91. In some embodiments, the antibody comprises a CDR H1 of SEQ ID NO: 71, a CDR H3 of SEQ ID NO: 79, and a CDR L1 of SEQ ID NO: 86. In some embodiments, the antibody comprises a CDR H1 of SEQ ID NO: 71, a CDR H3 of SEQ ID NO: 79, and a CDR L2 of SEQ ID NO: 90. In some embodiments, the antibody comprises a CDR H1 of SEQ ID NO: 71, a CDR H3 of SEQ ID NO: 79, and a CDR L3 of SEQ ID NO: 91. In some embodiments, the antibody comprises a CDR H2 of SEQ ID NO: 78, a CDR H3 of SEQ ID NO: 79, and a CDR L1 of SEQ ID NO: 86. In some embodiments, the antibody comprises a CDR H2 of SEQ ID NO: 78, a CDR H3 of SEQ ID NO: 79, and a CDR L2 of SEQ ID NO: 90. In some embodiments, the antibody comprises a CDR H2 of SEQ ID NO: 78, a CDR H3 of SEQ ID NO: 79, and a CDR L3 of SEQ ID NO: 91.

In some embodiments, the antibody comprises a CDR H1 of SEQ ID NO: 71, a CDR L1 of SEQ ID NO: 86, and a CDR L2 of SEQ ID NO: 90. In some embodiments, the antibody comprises a CDR H1 of SEQ ID NO: 71, a CDR L1 of SEQ ID NO: 86, and a CDR L3 of SEQ ID NO: 91. In some embodiments, the antibody comprises a CDR H1 of SEQ ID NO: 71, a CDR L2 of SEQ ID NO: 90, and a CDR L3 of SEQ ID NO: 91. In some embodiments, the antibody comprises a CDR H2 of SEQ ID NO: 78, a CDR L1 of SEQ ID NO: 86, and a CDR L2 of SEQ ID NO: 90. In some embodiments, the antibody comprises a CDR H2 of SEQ ID NO: 78, a CDR L1 of SEQ ID NO: 86, and a CDR L3 of SEQ ID NO: 91. In some embodiments, the antibody comprises a CDR H2 of SEQ ID NO: 78, a CDR L2 of SEQ ID NO: 90, and a CDR L3 of SEQ ID NO: 91. In some embodiments, the antibody comprises a CDR H3 of SEQ ID NO: 79, a CDR L1 of SEQ ID NO: 86, and a CDR L2 of SEQ ID NO: 90. In some embodiments, the antibody comprises a CDR H3 of SEQ ID NO: 79, a CDR L1 of SEQ ID NO: 86, and a CDR L3 of SEQ ID NO: 91. In some embodiments, the antibody comprises a CDR H3 of SEQ ID NO: 79, a CDR L2 of SEQ ID NO: 90, and a CDR L3 of SEQ ID NO: 91.

In some embodiments, the antibody comprises a CDR H3 of SEQ ID NO: 79, a CDR L1 of SEQ ID NO: 86, a CDR L2 of SEQ ID NO: 90, and a CDR L3 of SEQ ID NO: 91. In some embodiments, the antibody comprises a CDR H2 of SEQ ID NO: 78, a CDR L1 of SEQ ID NO: 86, a CDR L2 of SEQ ID NO: 90, and a CDR L3 of SEQ ID NO: 91. In some embodiments, the antibody comprises a CDR H1 of SEQ ID NO: 71, a CDR L1 of SEQ ID NO: 86, a CDR L2 of SEQ ID NO: 90, and a CDR L3 of SEQ ID NO: 91. In some embodiments, the antibody comprises a CDR H1 of SEQ ID NO: 71, a CDR H2 of SEQ ID NO: 78, a CDR H3 of SEQ ID NO: 79, and a CDR L3 of SEQ ID NO: 91. In some embodiments, the antibody comprises a CDR H1 of SEQ ID NO: 71, a CDR H2 of SEQ ID NO: 78, a CDR H3 of SEQ ID NO: 79, and a CDR L2 of SEQ ID NO: 90. In some embodiments, the antibody comprises a CDR H1 of SEQ ID NO: 71, a CDR H2 of SEQ ID NO: 78, a CDR H3 of SEQ ID NO: 79, and a CDR L1 of SEQ ID NO: 86.

In some embodiments, the antibody comprises a CDR H2 of SEQ ID NO: 78, a CDR H3 of SEQ ID NO: 79, a CDR L2 of SEQ ID NO: 90, and a CDR L3 of SEQ ID NO: 91. In some embodiments, the antibody comprises a CDR H2 of SEQ ID NO: 78, a CDR H3 of SEQ ID NO: 79, a CDR L1 of SEQ ID NO: 86, and a CDR L3 of SEQ ID NO: 91. In some embodiments, the antibody comprises a CDR H2 of SEQ ID NO: 78, a CDR H3 of SEQ ID NO: 79, a CDR L1 of SEQ ID NO: 86, and a CDR L2 of SEQ ID NO: 90. In some embodiments, the antibody comprises a CDR H1 of SEQ ID NO: 71, a CDR H3 of SEQ ID NO: 79, a CDR L2 of SEQ ID NO: 90, and a CDR L3 of SEQ ID NO: 91. In some embodiments, the antibody comprises a CDR H1 of SEQ ID NO: 71, a CDR H3 of SEQ ID NO: 79, a CDR L1 of SEQ ID NO: 86, and a CDR L3 of SEQ ID NO: 91. In some embodiments, the antibody comprises a CDR H1 of SEQ ID NO: 71, a CDR H3 of SEQ ID NO: 79, a CDR L1 of SEQ ID NO: 86, and a CDR L2 of SEQ ID NO: 90. In some embodiments, the antibody comprises a CDR H1 of SEQ ID NO: 71, a CDR H2 of SEQ ID NO: 78, a CDR L2 of SEQ ID NO: 90, and a CDR L3 of SEQ ID NO: 91. In some embodiments, the antibody comprises a CDR H1 of SEQ ID NO: 71, a CDR H2 of SEQ ID NO: 78, a CDR L1 of SEQ ID NO: 86, and a CDR L3 of SEQ ID NO: 91.

In some embodiments, the antibody comprises a CDR H1 of SEQ ID NO: 71, a CDR H2 of SEQ ID NO: 78, a CDR L1 of SEQ ID NO: 86, and a CDR L2 of SEQ ID NO: 90.

In some embodiments, the antibody comprises a CDR H1 of SEQ ID NO: 71, a CDR H2 of SEQ ID NO: 78, a CDR H3 of SEQ ID NO: 79, a CDR L2 of SEQ ID NO: 90, and a CDR L3 of SEQ ID NO: 91. In some embodiments, the antibody comprises a CDR H1 of SEQ ID NO: 71, a CDR H2 of SEQ ID NO: 78, a CDR H3 of SEQ ID NO: 79, a CDR L1 of SEQ ID NO: 86, and a CDR L3 of SEQ ID NO: 91. In some embodiments, the antibody comprises a CDR H1 of SEQ ID NO: 71, a CDR H2 of SEQ ID NO: 78, a CDR H3 of SEQ ID NO: 79, a CDR L1 of SEQ ID NO: 86, and a CDR L2 of SEQ ID NO: 90. In some embodiments, the antibody comprises a CDR H2 of SEQ ID NO: 78, a CDR H3 of SEQ ID NO: 79, a CDR L1 of SEQ ID NO: 86, a CDR L2 of SEQ ID NO: 90, and a CDR L3 of SEQ ID NO: 91. In some embodiments, the antibody comprises a CDR H1 of SEQ ID NO: 71, a CDR H3 of SEQ ID NO: 79, a CDR L1 of SEQ ID NO: 86, a CDR L2 of SEQ ID NO: 90, and a CDR L3 of SEQ ID NO: 91. In some embodiments, the antibody comprises a CDR H1 of SEQ ID NO: 71, a CDR H2 of SEQ ID NO: 78, a CDR L1 of SEQ ID NO: 86, a CDR L2 of SEQ ID NO: 90, and a CDR L3 of SEQ ID NO: 91.

In a specific embodiment, the antibody comprises a CDR H1 of SEQ ID NO: 71, a CDR H2 of SEQ ID NO: 78, a CDR H3 of SEQ ID NO: 79, a CDR L1 of SEQ ID NO: 86, a CDR L2 of SEQ ID NO: 90, and a CDR L3 of SEQ ID NO: 91.

In some embodiments, the antibody provided herein comprises one or more CDR regions from antibody 144J171G.

In some embodiments, the antibody comprises a CDR H1 having an amino acid sequence of the CDR H1 contained in SEQ ID NO: 47. In some embodiments, the antibody comprises a CDR H2 having an amino acid sequence of the CDR H2 contained in SEQ ID NO: 47. In some embodiments, the antibody comprises a CDR H3 having an amino acid sequence of the CDR H3 contained in SEQ ID NO: 47. In some embodiments, the antibody comprises a CDR L1 having an amino acid sequence of the CDR L1 contained in SEQ ID NO: 67. In some embodiments, the antibody comprises a CDR L2 having an amino acid sequence of the CDR L2 contained in SEQ ID NO: 67. In some embodiments, the antibody comprises a CDR L3 having an amino acid sequence of the CDRL3 contained in SEQ ID NO: 67.

In some embodiments, the antibody comprises a CDR H1 and a CDR H2 having amino acid sequences of the CDR H1 and the CDR H2 contained in SEQ ID NO: 47. In some embodiments, the antibody comprises a CDR H1 and a CDR H3 having amino acid sequences of the CDR H1 and the CDR H3 contained in SEQ ID NO: 47. In some embodiments, the antibody comprises a CDR H2 and a CDR H3 having amino acid sequences of the CDR H2 and the CDR H3 contained in SEQ ID NO: 47. In some embodiments, the antibody comprises a CDR L1 and a CDR L2 having amino acid sequences of the CDR L1 and the CDR L2 contained in SEQ ID NO: 67. In some embodiments, the antibody comprises a CDR L1 and a CDR L3 having amino acid sequences of the CDR L1 and the CDR L3 contained in SEQ ID NO: 67. In some embodiments, the antibody comprises a CDR L2 and a CDR L3 having amino acid sequences of the CDR L2 and the CDR L3 contained in SEQ ID NO: 67.

In some embodiments, the antibody comprises a CDR H1 and a CDR L1 having amino acid sequences of the CDR H1 and the CDR L1 contained in SEQ ID NO: 47 and SEQ ID NO: 67 respectively. In some embodiments, the antibody comprises a CDR H1 and a CDR L2 having amino acid sequences of the CDR H1 and the CDR L2 contained in SEQ ID NO: 47 and SEQ ID NO: 67 respectively. In some embodiments, the antibody comprises a CDR H1 and a CDR L3 having amino acid sequences of the CDR H1 and the CDR L3 contained in SEQ ID NO: 47 and SEQ ID NO: 67 respectively. In some embodiments, the antibody comprises a CDR H2 and a CDR L1 having amino acid sequences of the CDR H2 and the CDR L1 contained in SEQ ID NO: 47 and SEQ ID NO: 67 respectively. In some embodiments, the antibody comprises a CDR H2 and a CDR L2 having amino acid sequences of the CDR H2 and the CDR L2 contained in SEQ ID NO: 47 and SEQ ID NO: 67 respectively. In some embodiments, the antibody comprises a CDR H2 and a CDR L3 having amino acid sequences of the CDR H2 and the CDR L3 contained in SEQ ID NO: 47 and SEQ ID NO: 67 respectively. In some embodiments, the antibody comprises a CDR H3 and a CDR L1 having amino acid sequences of the CDR H3 and the CDR L1 contained in SEQ ID NO: 47 and SEQ ID NO: 67 respectively. In some embodiments, the antibody comprises a CDR H3 and a CDR L2 having amino acid sequences of the CDR H3 and the CDR L2 contained in SEQ ID NO: 47 and SEQ ID NO: 67 respectively. In some embodiments, the antibody comprises a CDR H3 and a CDR L3 having amino acid sequences of the CDR H3 and the CDR L3 contained in SEQ ID NO: 47 and SEQ ID NO: 67 respectively.

In some embodiments, the antibody comprises a CDR H1, a CDR H2, and a CDR H3 having amino acid sequences of the CDR H1, the CDR H2, and the CDR H3 contained in SEQ ID NO: 47. In some embodiments, the antibody comprises a CDR L1, a CDR L2, and a CDR L3 having amino acid sequences of the CDR L1, the CDR L2, and the CDR L3 contained in SEQ ID NO: 67.

In some embodiments, the antibody comprises a CDR H1, a CDR H2, and a CDR L1 having amino acid sequences of the CDR H1, the CDR H2, and the CDR L1 contained in SEQ ID NO: 47 and SEQ ID NO: 67 respectively. In some embodiments, the antibody comprises a CDR H1, a CDR H2, and a CDR L2 having amino acid sequences of the CDR H1, the CDR H2, and the CDR L2 contained in SEQ ID NO: 47 and SEQ ID NO: 67 respectively. In some embodiments, the antibody comprises a CDR H1, a CDR H2, and a CDR L3 having amino acid sequences of the CDR H1, the CDR H2, and the CDR L3 contained in SEQ ID NO: 47 and SEQ ID NO: 67 respectively.

In some embodiments, the antibody comprises a CDR H1, a CDR H3, and a CDR L1 having amino acid sequences of the CDR H1, the CDR H3, and the CDR L1 contained in SEQ ID NO: 47 and SEQ ID NO: 67 respectively. In some embodiments, the antibody comprises a CDR H1, a CDR H3, and a CDR L2 having amino acid sequences of the CDR H1, the CDR H3, and the CDR L2 contained in SEQ ID NO: 47 and SEQ ID NO: 67 respectively. In some embodiments, the antibody comprises a CDR H1, a CDR H3, and a CDR L3 having amino acid sequences of the CDR H1, the CDR H3, and the CDR L3 contained in SEQ ID NO: 47 and SEQ ID NO: 67 respectively.

In some embodiments, the antibody comprises a CDR H2, a CDR H3, and a CDR L1 having amino acid sequences of the CDR H2, the CDR H3, and the CDR L1 contained in SEQ ID NO: 47 and SEQ ID NO: 67 respectively. In some embodiments, the antibody comprises a CDR H2, a CDR H3, and a CDR L2 having amino acid sequences of the CDR H2, the CDR H3, and the CDR L2 contained in SEQ ID NO: 47 and SEQ ID NO: 67 respectively. In some embodiments, the antibody comprises a CDR H2, a CDR H3, and a CDR L3 having amino acid sequences of the CDR H2, the CDR H3, and the CDR L3 contained in SEQ ID NO: 47 and SEQ ID NO: 67 respectively.

In some embodiments, the antibody comprises a CDR H1, a CDR L1, and a CDR L2 having amino acid sequences of the CDR H1, the CDR L1, and the CDR L2 contained in SEQ ID NO: 47 and SEQ ID NO: 67 respectively. In some embodiments, the antibody comprises a CDR H1, a CDR L1, and a CDR L3 having amino acid sequences of the CDR H1, the CDR L1, and the CDR L3 contained in SEQ ID NO: 47 and SEQ ID NO: 67 respectively. In some embodiments, the antibody comprises a CDR H1, a CDR L2, and a CDR L3 having amino acid sequences of the CDR H1, the CDR L2, and the CDR L3 contained in SEQ ID NO: 47 and SEQ ID NO: 67 respectively.

In some embodiments, the antibody comprises a CDR H2, a CDR L1, and a CDR L2 having amino acid sequences of the CDR H2, the CDR L1, and the CDR L2 contained in SEQ ID NO: 47 and SEQ ID NO: 67 respectively. In some embodiments, the antibody comprises a CDR H2, a CDR L1, and a CDR L3 having amino acid sequences of the CDR H2, the CDR L1, and the CDR L3 contained in SEQ ID NO: 47 and SEQ ID NO: 67 respectively. In some embodiments, the antibody comprises a CDR H2, a CDR L2, and a CDR L3 having amino acid sequences of the CDR H2, the CDR L2, and the CDR L3 contained in SEQ ID NO: 47 and SEQ ID NO: 67 respectively.

In some embodiments, the antibody comprises a CDR H3, a CDR L1, and a CDR L2 having amino acid sequences of the CDR H3, the CDR L1, and the CDR L2 contained in SEQ ID NO: 47 and SEQ ID NO: 67 respectively. In some embodiments, the antibody comprises a CDR H3, a CDR L1, and a CDR L3 having amino acid sequences of the CDR H3, the CDR L1, and the CDR L3 contained in SEQ ID NO: 47 and SEQ ID NO: 67 respectively. In some embodiments, the antibody comprises a CDR H3, a CDR L2, and a CDR L3 having amino acid sequences of the CDR H3, the CDR L2, and the CDR L3 contained in SEQ ID NO: 47 and SEQ ID NO: 67 respectively.

In some embodiments, the antibody comprises a CDR H3, a CDR L1, a CDR L2 and a CDR L3 having amino acid sequences of the CDR H3, the CDR L1, the CDR L2 and the CDR L3 contained in SEQ ID NO: 47 and SEQ ID NO: 67 respectively. In some embodiments, the antibody comprises a CDR H2, a CDR L1, a CDR L2 and a CDR L3 having amino acid sequences of the CDR H2, the CDR L1, the CDR L2 and the CDR L3 contained in SEQ ID NO: 47 and SEQ ID NO: 67 respectively. In some embodiments, the antibody comprises a CDR H1, a CDR L1, a CDR L2 and a CDR L3 having amino acid sequences of the CDR H1, the CDR L1, the CDR L2 and the CDR L3 contained in SEQ ID NO: 47 and SEQ ID NO: 67 respectively. In some embodiments, the antibody comprises a CDR H1, a CDR H2, a CDR H3 and a CDR L3 having amino acid sequences of the CDR H1, the CDR H2, the CDR H3 and the CDR L3 contained in SEQ ID NO: 47 and SEQ ID NO: 67 respectively. In some embodiments, the antibody comprises a CDR H1, a CDR H2, a CDR H3 and a CDR L2 having amino acid sequences of the CDR H1, the CDR H2, the CDR H3 and the CDR L2 contained in SEQ ID NO: 47 and SEQ ID NO: 67 respectively. In some embodiments, the antibody comprises a CDR H1, a CDR H2, a CDR H3 and a CDR L1 having amino acid sequences of the CDR H1, the CDR H2, the CDR H3 and the CDR L1 contained in SEQ ID NO: 47 and SEQ ID NO: 67 respectively.

In some embodiments, the antibody comprises a CDR H2, a CDR H3, a CDR L2 and a CDR L3 having amino acid sequences of the CDR H2, the CDR H3, the CDR L2 and the CDR L3 contained in SEQ ID NO: 47 and SEQ ID NO: 67 respectively. In some embodiments, the antibody comprises a CDR H2, a CDR H3, a CDR L1 and a CDR L3 having amino acid sequences of the CDR H2, the CDR H3, the CDR L1 and the CDR L3 contained in SEQ ID NO: 47 and SEQ ID NO: 67 respectively. In some embodiments, the antibody comprises a CDR H2, a CDR H3, a CDR L1 and a CDR L2 having amino acid sequences of the CDR H2, the CDR H3, the CDR L1 and the CDR L2 contained in SEQ ID NO: 47 and SEQ ID NO: 67 respectively. In some embodiments, the antibody comprises a CDR H1, a CDR H3, a CDR L2 and a CDR L3 having amino acid sequences of the CDR H1, the CDR H3, the CDR L2 and the CDR L3 contained in SEQ ID NO: 47 and SEQ ID NO: 67 respectively. In some embodiments, the antibody comprises a CDR H1, a CDR H3, a CDR L1 and a CDR L3 having amino acid sequences of the CDR H1, the CDR H3, the CDR L1 and the CDR L3 contained in SEQ ID NO: 47 and SEQ ID NO: 67 respectively. In some embodiments, the antibody comprises a CDR H1, a CDR H3, a CDR L1 and a CDR L2 having amino acid sequences of the CDR H1, the CDR H3, the CDR L1 and the CDR L2 contained in SEQ ID NO: 47 and SEQ ID NO: 67 respectively. In some embodiments, the antibody comprises a CDR H1, a CDR H2, a CDR L2 and a CDR L3 having amino acid sequences of the CDR H1, the CDR H2, the CDR L2 and the CDR L3 contained in SEQ ID NO: 47 and SEQ ID NO: 67 respectively. In some embodiments, the antibody comprises a CDR H1, a CDR H2, a CDR L1 and a CDR L3 having amino acid sequences of the CDR H1, the CDR H2, the CDR L1 and the CDR L3 contained in SEQ ID NO: 47 and SEQ ID NO: 67 respectively. In some embodiments, the antibody comprises a CDR H1, a CDR H2, a CDR L1 and a CDR L2 having amino acid sequences of the CDR H1, the CDR H2, the CDR L1 and the CDR L2 contained in SEQ ID NO: 47 and SEQ ID NO: 67 respectively.

In some embodiments, the antibody comprises a CDR H2, a CDR H3, a CDR L1, a CDR L2 and a CDR L3 having amino acid sequences of the CDR H2, the CDR H3, the CDR L1, the CDR L2 and the CDR L3 contained in SEQ ID NO: 47 and SEQ ID NO: 67 respectively. In some embodiments, the antibody comprises a CDR H1, a CDR H3, a CDR L1, a CDR L2 and a CDR L3 having amino acid sequences of the CDR H1, the CDR H3, the CDR L1, the CDR L2 and the CDR L3 contained in SEQ ID NO: 47 and SEQ ID NO: 67 respectively. In some embodiments, the antibody comprises a CDR H1, a CDR H2, a CDR L1, a CDR L2 and a CDR L3 having amino acid sequences of the CDR H1, the CDR H2, the CDR L1, the CDR L2 and the CDR L3 contained in SEQ ID NO: 47 and SEQ ID NO: 67 respectively. In some embodiments, the antibody comprises a CDR H1, a CDR H2, a CDR H3, a CDR L2 and a CDR L3 having amino acid sequences of the CDR H1, the CDR H2, the CDR H3, the CDR L2 and the CDR L3 contained in SEQ ID NO: 47 and SEQ ID NO: 67 respectively. In some embodiments, the antibody comprises a CDR H1, a CDR H2, a CDR H3, a CDR L1, and a CDR L3 having amino acid sequences of the CDR H1, the CDR H2, the CDR H3, the CDR L1, and the CDR L3 contained in SEQ ID NO: 47 and SEQ ID NO: 67 respectively. In some embodiments, the antibody comprises a CDR H1, a CDR H2, a CDR H3, a CDR L1, and a CDR L2 having amino acid sequences of the CDR H1, the CDR H2, the CDR H3, the CDR L1, and the CDR L2 contained in SEQ ID NO: 47 and SEQ ID NO: 67 respectively.

In some embodiments, the antibody comprises a CDR H1, a CDR H2, a CDR H3, a CDR L1, a CDR L2 and a CDR L3 having amino acid sequences of the CDR H1, the CDR H2, the CDR H3, the CDR L1, the CDR L2 and the CDR L3 contained in SEQ ID NO: 47 and SEQ ID NO: 67 respectively.

The residues from each of CDRs are noted in Table 27. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDRs are according to IMGT numbering.

In some embodiments, the CDRs are according to Kabat numbering. In some embodiments, the antibody comprises a CDR H1 of SEQ ID NO: 80. In some embodiments, the antibody comprises a CDR H2 of SEQ ID NO: 81. In some embodiments, the antibody comprises a CDR H3 of SEQ ID NO: 82. In some embodiments, the antibody comprises a CDR L1 of SEQ ID NO: 86. In some embodiments, the antibody comprises a CDR L2 of SEQ ID NO: 87. In other embodiments, the antibody comprises a CDR L3 of SEQ ID NO: 92.

In some embodiments, the antibody comprises a CDR H1 of SEQ ID NO: 80 and a CDR H2 of SEQ ID NO: 81. In some embodiments, the antibody comprises a CDR H1 of SEQ ID NO: 80 and a CDR H3 of SEQ ID NO: 82. In some embodiments, the antibody comprises a CDR H2 of SEQ ID NO: 81 and a CDR H3 of SEQ ID NO: 82. In some embodiments, the antibody comprises a CDR L1 of SEQ ID NO: 86 and a CDR L2 of SEQ ID NO: 87. In some embodiments, the antibody comprises a CDR L1 of SEQ ID NO: 86 and a CDR L3 of SEQ ID NO: 92. In some embodiments, the antibody comprises a CDR L2 of SEQ ID NO: 87 and a CDR L3 of SEQ ID NO: 92.

In some embodiments, the antibody comprises a CDR H1 of SEQ ID NO: 80 and a CDR L1 of SEQ ID NO: 86. In some embodiments, the antibody comprises a CDR H1 of SEQ ID NO: 80 and a CDR L2 of SEQ ID NO: 87. In some embodiments, the antibody comprises a CDR H1 of SEQ ID NO: 80 and a CDR L3 of SEQ ID NO: 92. In some embodiments, the antibody comprises a CDR H2 of SEQ ID NO: 81 and a CDR L1 of SEQ ID NO: 86. In some embodiments, the antibody comprises a CDR H2 of SEQ ID NO: 81 and a CDR L2 of SEQ ID NO: 87. In some embodiments, the Ab comprises a CDR H2 of SEQ ID NO: 81 and a CDRL3 of SEQ ID NO: 92. In some embodiments, the antibody comprises a CDR H3 of SEQ ID NO: 82 and a CDR L1 of SEQ ID NO: 86. In some embodiments, the antibody comprises a CDR H3 of SEQ ID NO: 82 and a CDR L2 of SEQ ID NO: 87. In some embodiments, the antibody comprises a CDR H3 of SEQ ID NO: 82 and a CDR L3 of SEQ ID NO: 92.

In some embodiments, the antibody comprises a CDR H1 of SEQ ID NO: 80, a CDR H2 of SEQ ID NO: 81, and a CDR H3 of SEQ ID NO: 82. In some embodiments, the antibody comprises a CDR L1 of SEQ ID NO: 86, a CDR L2 of SEQ ID NO: 87, and a CDR L3 of SEQ ID NO: 92.

In some embodiments, the antibody comprises a CDR H1 of SEQ ID NO: 80, a CDR H2 of SEQ ID NO: 81, and a CDR L1 of SEQ ID NO: 86. In some embodiments, the antibody comprises a CDR H1 of SEQ ID NO: 80, a CDR H2 of SEQ ID NO: 81, and a CDR L2 of SEQ ID NO: 87. In some embodiments, the antibody comprises a CDR H1 of SEQ ID NO: 80, a CDR H2 of SEQ ID NO: 81, and a CDR L3 of SEQ ID NO: 92. In some embodiments, the antibody comprises a CDR H1 of SEQ ID NO: 80, a CDR H3 of SEQ ID NO: 82, and a CDR L1 of SEQ ID NO: 86. In some embodiments, the antibody comprises a CDR H1 of SEQ ID NO: 80, a CDR H3 of SEQ ID NO: 82, and a CDR L2 of SEQ ID NO: 87. In some embodiments, the antibody comprises a CDR H1 of SEQ ID NO: 80, a CDR H3 of SEQ ID NO: 82, and a CDR L3 of SEQ ID NO: 92. In some embodiments, the antibody comprises a CDR H2 of SEQ ID NO: 81, a CDR H3 of SEQ ID NO: 82, and a CDR L1 of SEQ ID NO: 86. In some embodiments, the antibody comprises a CDR H2 of SEQ ID NO: 81, a CDR H3 of SEQ ID NO: 82, and a CDR L2 of SEQ ID NO: 87. In some embodiments, the antibody comprises a CDR H2 of SEQ ID NO: 81, a CDR H3 of SEQ ID NO: 82, and a CDR L3 of SEQ ID NO: 92.

In some embodiments, the antibody comprises a CDR H1 of SEQ ID NO: 80, a CDR L1 of SEQ ID NO: 86, and a CDR L2 of SEQ ID NO: 87. In some embodiments, the antibody comprises a CDR H1 of SEQ ID NO: 80, a CDR L1 of SEQ ID NO: 86, and a CDR L3 of SEQ ID NO: 92. In some embodiments, the antibody comprises a CDR H1 of SEQ ID NO: 80, a CDR L2 of SEQ ID NO: 87, and a CDR L3 of SEQ ID NO: 92. In some embodiments, the antibody comprises a CDR H2 of SEQ ID NO: 81, a CDR L1 of SEQ ID NO: 86, and a CDR L2 of SEQ ID NO: 87. In some embodiments, the antibody comprises a CDR H2 of SEQ ID NO: 81, a CDR L1 of SEQ ID NO: 86, and a CDR L3 of SEQ ID NO: 92. In some embodiments, the antibody comprises a CDR H2 of SEQ ID NO: 81, a CDR L2 of SEQ ID NO: 87, and a CDR L3 of SEQ ID NO: 92. In some embodiments, the antibody comprises a CDR H3 of SEQ ID NO: 82, a CDR L1 of SEQ ID NO: 86, and a CDR L2 of SEQ ID NO: 87. In some embodiments, the antibody comprises a CDR H3 of SEQ ID NO: 82, a CDR L1 of SEQ ID NO: 86, and a CDR L3 of SEQ ID NO: 92. In some embodiments, the antibody comprises a CDR H3 of SEQ ID NO: 82, a CDR L2 of SEQ ID NO: 87, and a CDR L3 of SEQ ID NO: 92.

In some embodiments, the antibody comprises a CDR H3 of SEQ ID NO: 82, a CDR L1 of SEQ ID NO: 86, a CDR L2 of SEQ ID NO: 87, and a CDR L3 of SEQ ID NO: 92. In some embodiments, the antibody comprises a CDR H2 of SEQ ID NO: 81, a CDR L1 of SEQ ID NO: 86, a CDR L2 of SEQ ID NO: 87, and a CDR L3 of SEQ ID NO: 92. In some embodiments, the antibody comprises a CDR H1 of SEQ ID NO: 80, a CDR L1 of SEQ ID NO: 86, a CDR L2 of SEQ ID NO: 87, and a CDR L3 of SEQ ID NO: 92. In some embodiments, the antibody comprises a CDR H1 of SEQ ID NO: 80, a CDR H2 of SEQ ID NO: 81, a CDR H3 of SEQ ID NO: 82, and a CDR L3 of SEQ ID NO: 92. In some embodiments, the antibody comprises a CDR H1 of SEQ ID NO: 80, a CDR H2 of SEQ ID NO: 81, a CDR H3 of SEQ ID NO: 82, and a CDR L2 of SEQ ID NO: 87. In some embodiments, the antibody comprises a CDR H1 of SEQ ID NO: 80, a CDR H2 of SEQ ID NO: 81, a CDR H3 of SEQ ID NO: 82, and a CDR L1 of SEQ ID NO: 86.

In some embodiments, the antibody comprises a CDR H2 of SEQ ID NO: 81, a CDR H3 of SEQ ID NO: 82, a CDR L2 of SEQ ID NO: 87, and a CDR L3 of SEQ ID NO: 92. In some embodiments, the antibody comprises a CDR H2 of SEQ ID NO: 81, a CDR H3 of SEQ ID NO: 82, a CDR L1 of SEQ ID NO: 86, and a CDR L3 of SEQ ID NO: 92. In some embodiments, the antibody comprises a CDR H2 of SEQ ID NO: 81, a CDR H3 of SEQ ID NO: 82, a CDR L1 of SEQ ID NO: 86, and a CDR L2 of SEQ ID NO: 87. In some embodiments, the antibody comprises a CDR H1 of SEQ ID NO: 80, a CDR H3 of SEQ ID NO: 82, a CDR L2 of SEQ ID NO: 87, and a CDR L3 of SEQ ID NO: 92. In some embodiments, the antibody comprises a CDR H1 of SEQ ID NO: 80, a CDR H3 of SEQ ID NO: 82, a CDR L1 of SEQ ID NO: 86, and a CDR L3 of SEQ ID NO: 92. In some embodiments, the antibody comprises a CDR H1 of SEQ ID NO: 80, a CDR H3 of SEQ ID NO: 82, a CDR L1 of SEQ ID NO: 86, and a CDR L2 of SEQ ID NO: 87. In some embodiments, the antibody comprises a CDR H1 of SEQ ID NO: 80, a CDR H2 of SEQ ID NO: 81, a CDR L2 of SEQ ID NO: 87, and a CDR L3 of SEQ ID NO: 92. In some embodiments, the antibody comprises a CDR H1 of SEQ ID NO: 80, a CDR H2 of SEQ ID NO: 81, a CDR L1 of SEQ ID NO: 86, and a CDR L3 of SEQ ID NO: 92. In some embodiments, the antibody comprises a CDR H1 of SEQ ID NO: 80, a CDR H2 of SEQ ID NO: 81, a CDR L1 of SEQ ID NO: 86, and a CDR L2 of SEQ ID NO: 87.

In some embodiments, the antibody comprises a CDR H1 of SEQ ID NO: 80, a CDR H2 of SEQ ID NO: 81, a CDR H3 of SEQ ID NO: 82, a CDR L2 of SEQ ID NO: 87, and a CDR L3 of SEQ ID NO: 92. In some embodiments, the antibody comprises a CDR H1 of SEQ ID NO: 80, a CDR H2 of SEQ ID NO: 81, a CDR H3 of SEQ ID NO: 82, a CDR L1 of SEQ ID NO: 86, and a CDR L3 of SEQ ID NO: 92. In some embodiments, the antibody comprises a CDR H1 of SEQ ID NO: 80, a CDR H2 of SEQ ID NO: 81, a CDR H3 of SEQ ID NO: 82, a CDR L1 of SEQ ID NO: 86, and a CDR L2 of SEQ ID NO: 87. In some embodiments, the antibody comprises a CDR H2 of SEQ ID NO: 81, a CDR H3 of SEQ ID NO: 82, a CDR L1 of SEQ ID NO: 86, a CDR L2 of SEQ ID NO: 87, and a CDR L3 of SEQ ID NO: 92. In some embodiments, the antibody comprises a CDR H1 of SEQ ID NO: 80, a CDR H3 of SEQ ID NO: 82, a CDR L1 of SEQ ID NO: 86, a CDR L2 of SEQ ID NO: 87, and a CDR L3 of SEQ ID NO: 92. In some embodiments, the antibody comprises a CDR H1 of SEQ ID NO: 80, a CDR H2 of SEQ ID NO: 81, a CDR L1 of SEQ ID NO: 86, a CDR L2 of SEQ ID NO: 87, and a CDR L3 of SEQ ID NO: 92.

In a specific embodiment, the antibody comprises a CDR H1 of SEQ ID NO: 80, a CDR H2 of SEQ ID NO: 81, a CDR H3 of SEQ ID NO: 82, a CDR L1 of SEQ ID NO: 86, a CDR L2 of SEQ ID NO: 87, and a CDR L3 of SEQ ID NO: 92.

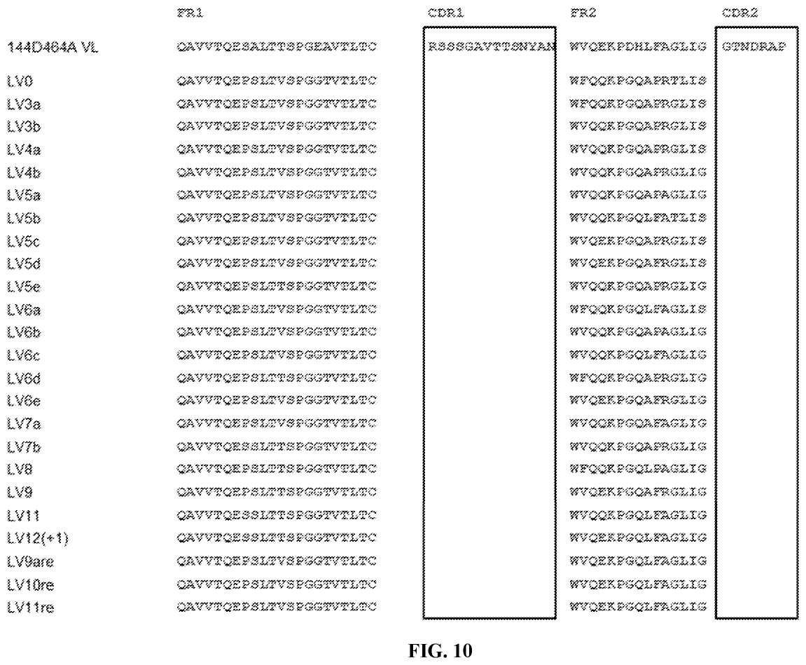

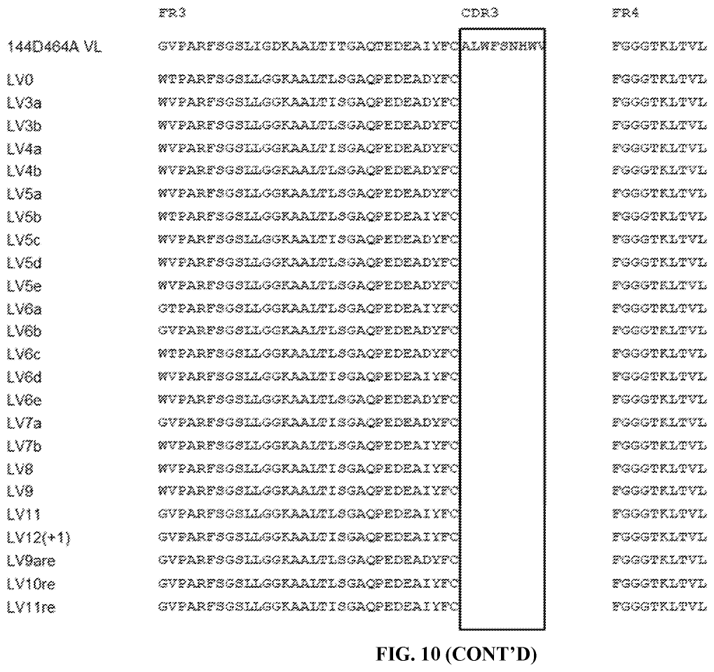

In some embodiments, the antibody provided herein comprises one or more CDR sequences of the humanized antibodies provided in Section 6 below. In some embodiments, the antibody provided herein comprises one or more CDR sequences shown in FIGS. 10-13. In a specific embodiment, the antibody comprises a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 84, and a CDR L3 of SEQ ID NO: 88. In another specific embodiment, the antibody comprises a CDR H1 of SEQ ID NO: 68, a CDR H2 of SEQ ID NO: 69, a CDR H3 of SEQ ID NO: 70, a CDR L1 of SEQ ID NO: 83, a CDR L2 of SEQ ID NO: 84, and a CDR L3 of SEQ ID NO: 85.

In certain embodiments, the antibody or antigen binding fragment thereof provided herein further comprises one or more FR regions from antibody 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c.

In some embodiments, the antibody or antigen binding fragment thereof further comprises heavy chain FR regions contained in SEQ ID NO: 23, SEQ ID NO: 27, SEQ ID NO: 31, SEQ ID NO: 35, SEQ ID NO: 39, SEQ ID NO: 43, or SEQ ID NO: 47, and/or light chain FR regions contained in SEQ ID NO: 51, SEQ ID NO: 55, SEQ ID NO: 59, SEQ ID NO: 63, or SEQ ID NO: 67.

In some embodiments, the antibody or antigen binding fragment thereof comprises heavy chain FR regions comprising amino acid sequences of the FR regions contained in SEQ ID NO: 23, and light chain FR regions comprising amino acid sequences of the FR regions contained in SEQ ID NO: 51.

In some embodiments, the antibody or antigen binding fragment thereof comprises heavy chain FR regions comprising amino acid sequences of the FR regions contained in SEQ ID NO: 27, and light chain FR regions comprising amino acid sequences of the FR regions contained in SEQ ID NO: 55.

In some embodiments, the antibody or antigen binding fragment thereof comprises heavy chain FR regions comprising amino acid sequences of the FR regions contained in SEQ ID NO: 31, and light chain FR regions comprising amino acid sequences of the FR regions contained in SEQ ID NO: 55.

In some embodiments, the antibody or antigen binding fragment thereof comprises heavy chain FR regions comprising amino acid sequences of the FR regions contained in SEQ ID NO: 35, and light chain FR regions comprising amino acid sequences of the FR regions contained in SEQ ID NO: 55.

In some embodiments, the antibody or antigen binding fragment thereof comprises heavy chain FR regions comprising amino acid sequences of the FR regions contained in SEQ ID NO: 39, and light chain FR regions comprising amino acid sequences of the FR regions contained in SEQ ID NO: 59.

In some embodiments, the antibody or antigen binding fragment thereof comprises heavy chain FR regions comprising amino acid sequences of the FR regions contained in SEQ ID NO: 43, and light chain FR regions comprising amino acid sequences of the FR regions contained in SEQ ID NO: 63.

In some embodiments, the antibody or antigen binding fragment thereof comprises heavy chain FR regions comprising amino acid sequences of the FR regions contained in SEQ ID NO: 47, and light chain FR regions comprising amino acid sequences of the FR regions contained in SEQ ID NO: 67.

In some embodiments, the antibody provided herein further comprises one or more FR regions in the heavy chain of 144D464A. In some embodiments, the antibody provided herein comprises a heavy chain FR1 having an amino acid sequence of the FR1 contained in SEQ ID NO: 23. In some embodiments, the antibody provided herein comprises a heavy chain FR2 having an amino acid sequence of the FR2 contained in SEQ ID NO: 23. In some embodiments, the antibody provided herein comprises a heavy chain FR3 having an amino acid sequence of the FR3 contained in SEQ ID NO: 23. In some embodiments, the antibody provided herein comprises a heavy chain FR4 having an amino acid sequence of the FR4 contained in SEQ ID NO: 23.

In some embodiments, the antibody provided herein comprises a heavy chain FR1 and a heavy chain FR2 having amino acid sequences of the FR1 and the FR2 contained in SEQ ID NO: 23. In some embodiments, the antibody provided herein comprises a heavy chain FR1 and a heavy chain FR3 having amino acid sequences of the FR1 and the FR3 contained in SEQ ID NO: 23. In some embodiments, the antibody provided herein comprises a heavy chain FR1 and a heavy chain FR4 having amino acid sequences of the FR1 and the FR4 contained in SEQ ID NO: 23. In some embodiments, the antibody provided herein comprises a heavy chain FR2 and a heavy chain FR3 having amino acid sequences of the FR2 and the FR3 contained in SEQ ID NO: 23. In some embodiments, the antibody provided herein comprises a heavy chain FR2 and a heavy chain FR4 having amino acid sequences of the FR2 and the FR4 contained in SEQ ID NO: 23. In some embodiments, the antibody provided herein comprises a heavy chain FR3 and a heavy chain FR4 having amino acid sequences of the FR3 and the FR4 contained in SEQ ID NO: 23.

In some embodiments, the antibody provided herein comprises a heavy chain FR1, a heavy chain FR2, and a heavy chain FR3 having amino acid sequences of the FR1, the FR2, and the FR3 contained in SEQ ID NO: 23. In some embodiments, the antibody provided herein comprises a heavy chain FR1, a heavy chain FR2, and a heavy chain FR4 having amino acid sequences of the FR1, the FR2, and the FR4 contained in SEQ ID NO: 23. In some embodiments, the antibody provided herein comprises a heavy chain FR1, a heavy chain FR3, and a heavy chain FR4 having amino acid sequences of the FR1, the FR3, and the FR4 contained in SEQ ID NO: 23. In some embodiments, the antibody provided herein comprises a heavy chain FR2, a heavy chain FR3, and a heavy chain FR4 having amino acid sequences of the FR2, the FR3, and the FR4 contained in SEQ ID NO: 23. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 23.

In some embodiments, the antibody provided herein further comprises one or more FR regions of the light chain FRs of 144D464A. In some embodiments, the antibody provided herein comprises a light chain FR1 having an amino acid sequence of the FR1 contained in SEQ ID NO: 51. In some embodiments, the antibody provided herein comprises a light chain FR2 having an amino acid sequence of the FR2 contained in SEQ ID NO: 51. In some embodiments, the antibody provided herein comprises a light chain FR3 having an amino acid sequence of the FR3 contained in SEQ ID NO: 51. In some embodiments, the antibody provided herein comprises a light chain FR4 having an amino acid sequence of the FR4 contained in SEQ ID NO: 51.

In some embodiments, the antibody provided herein comprises a light chain FR1 and a light chain FR2 having amino acid sequences of the FR1 and the FR2 contained in SEQ ID NO: 51. In some embodiments, the antibody provided herein comprises a light chain FR1 and a light chain FR3 having amino acid sequences of the FR1 and the FR3 contained in SEQ ID NO: 51. In some embodiments, the antibody provided herein comprises a light chain FR1 and a light chain FR4 having amino acid sequences of the FR1 and the FR4 contained in SEQ ID NO: 51. In some embodiments, the antibody provided herein comprises a light chain FR2 and a light chain FR3 having amino acid sequences of the FR2 and the FR3 contained in SEQ ID NO: 51. In some embodiments, the antibody provided herein comprises a light chain FR2 and a light chain FR4 having amino acid sequences of the FR2 and the FR4 contained in SEQ ID NO: 51. In some embodiments, the antibody provided herein comprises a light chain FR3 and a light chain FR4 having amino acid sequences of the FR3 and the FR4 contained in SEQ ID NO: 51.

In some embodiments, the antibody provided herein comprises a light chain FR1, a light chain FR2, and a light chain FR3 having amino acid sequences of the FR1, the FR2, and the FR3 contained in SEQ ID NO: 51. In some embodiments, the antibody provided herein comprises a light chain FR1, a light chain FR2, and a light chain FR4 having amino acid sequences of the FR1, the FR2, and the FR4 contained in SEQ ID NO: 51. In some embodiments, the antibody provided herein comprises a light chain FR1, a light chain FR3, and a light chain FR4 having amino acid sequences of the FR1, the FR3, and the FR4 contained in SEQ ID NO: 51. In some embodiments, the antibody provided herein comprises a light chain FR2, a light chain FR3, and a light chain FR4 having amino acid sequences of the FR2, the FR3, and the FR4 contained in SEQ ID NO: 51. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 51.

In some embodiments, the antibody provided herein further comprises one or more FR regions in the heavy chain of 144L249B. In some embodiments, the antibody provided herein comprises a heavy chain FR1 having an amino acid sequence of the FR1 contained in SEQ ID NO: 27. In some embodiments, the antibody provided herein comprises a heavy chain FR2 having an amino acid sequence of the FR2 contained in SEQ ID NO: 27. In some embodiments, the antibody provided herein comprises a heavy chain FR3 having an amino acid sequence of the FR3 contained in SEQ ID NO: 27. In some embodiments, the antibody provided herein comprises a heavy chain FR4 having an amino acid sequence of the FR4 contained in SEQ ID NO: 27.

In some embodiments, the antibody provided herein comprises a heavy chain FR1 and a heavy chain FR2 having amino acid sequences of the FR1 and the FR2 contained in SEQ ID NO: 27. In some embodiments, the antibody provided herein comprises a heavy chain FR1 and a heavy chain FR3 having amino acid sequences of the FR1 and the FR3 contained in SEQ ID NO: 27. In some embodiments, the antibody provided herein comprises a heavy chain FR1 and a heavy chain FR4 having amino acid sequences of the FR1 and the FR4 contained in SEQ ID NO: 27. In some embodiments, the antibody provided herein comprises a heavy chain FR2 and a heavy chain FR3 having amino acid sequences of the FR2 and the FR3 contained in SEQ ID NO: 27. In some embodiments, the antibody provided herein comprises a heavy chain FR2 and a heavy chain FR4 having amino acid sequences of the FR2 and the FR4 contained in SEQ ID NO: 27. In some embodiments, the antibody provided herein comprises a heavy chain FR3 and a heavy chain FR4 having amino acid sequences of the FR3 and the FR4 contained in SEQ ID NO: 27.

In some embodiments, the antibody provided herein comprises a heavy chain FR1, a heavy chain FR2, and a heavy chain FR3 having amino acid sequences of the FR1, the FR2, and the FR3 contained in SEQ ID NO: 27. In some embodiments, the antibody provided herein comprises a heavy chain FR1, a heavy chain FR2, and a heavy chain FR4 having amino acid sequences of the FR1, the FR2, and the FR4 contained in SEQ ID NO: 27. In some embodiments, the antibody provided herein comprises a heavy chain FR1, a heavy chain FR3, and a heavy chain FR4 having amino acid sequences of the FR1, the FR3, and the FR4 contained in SEQ ID NO: 27. In some embodiments, the antibody provided herein comprises a heavy chain FR2, a heavy chain FR3, and a heavy chain FR4 having amino acid sequences of the FR2, the FR3, and the FR4 contained in SEQ ID NO: 27. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 27.

In some embodiments, the antibody provided herein further comprises one or more FR regions of the light chain FRs of 144L249B. In some embodiments, the antibody provided herein comprises a light chain FR1 having an amino acid sequence of the FR1 contained in SEQ ID NO: 55. In some embodiments, the antibody provided herein comprises a light chain FR2 having an amino acid sequence of the FR2 contained in SEQ ID NO: 55. In some embodiments, the antibody provided herein comprises a light chain FR3 having an amino acid sequence of the FR3 contained in SEQ ID NO: 55. In some embodiments, the antibody provided herein comprises a light chain FR4 having an amino acid sequence of the FR4 contained in SEQ ID NO: 55.

In some embodiments, the antibody provided herein comprises a light chain FR1 and a light chain FR2 having amino acid sequences of the FR1 and the FR2 contained in SEQ ID NO: 55. In some embodiments, the antibody provided herein comprises a light chain FR1 and a light chain FR3 having amino acid sequences of the FR1 and the FR3 contained in SEQ ID NO: 55. In some embodiments, the antibody provided herein comprises a light chain FR1 and a light chain FR4 having amino acid sequences of the FR1 and the FR4 contained in SEQ ID NO: 55. In some embodiments, the antibody provided herein comprises a light chain FR2 and a light chain FR3 having amino acid sequences of the FR2 and the FR3 contained in SEQ ID NO: 55. In some embodiments, the antibody provided herein comprises a light chain FR2 and a light chain FR4 having amino acid sequences of the FR2 and the FR4 contained in SEQ ID NO: 55. In some embodiments, the antibody provided herein comprises a light chain FR3 and a light chain FR4 having amino acid sequences of the FR3 and the FR4 contained in SEQ ID NO: 55.

In some embodiments, the antibody provided herein comprises a light chain FR1, a light chain FR2, and a light chain FR3 having amino acid sequences of the FR1, the FR2, and the FR3 contained in SEQ ID NO: 55. In some embodiments, the antibody provided herein comprises a light chain FR1, a light chain FR2, and a light chain FR4 having amino acid sequences of the FR1, the FR2, and the FR4 contained in SEQ ID NO: 55. In some embodiments, the antibody provided herein comprises a light chain FR1, a light chain FR3, and a light chain FR4 having amino acid sequences of the FR1, the FR3, and the FR4 contained in SEQ ID NO: 55. In some embodiments, the antibody provided herein comprises a light chain FR2, a light chain FR3, and a light chain FR4 having amino acid sequences of the FR2, the FR3, and the FR4 contained in SEQ ID NO: 55. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 55.

In some embodiments, the antibody provided herein further comprises one or more FR regions in the heavy chain of 144L124B or 144L180A. In some embodiments, the antibody provided herein comprises a heavy chain FR1 having an amino acid sequence of the FR1 contained in SEQ ID NO: 31. In some embodiments, the antibody provided herein comprises a heavy chain FR2 having an amino acid sequence of the FR2 contained in SEQ ID NO: 31. In some embodiments, the antibody provided herein comprises a heavy chain FR3 having an amino acid sequence of the FR3 contained in SEQ ID NO: 31. In some embodiments, the antibody provided herein comprises a heavy chain FR4 having an amino acid sequence of the FR4 contained in SEQ ID NO: 31.

In some embodiments, the antibody provided herein comprises a heavy chain FR1 and a heavy chain FR2 having amino acid sequences of the FR1 and the FR2 contained in SEQ ID NO: 31. In some embodiments, the antibody provided herein comprises a heavy chain FR1 and a heavy chain FR3 having amino acid sequences of the FR1 and the FR3 contained in SEQ ID NO: 31. In some embodiments, the antibody provided herein comprises a heavy chain FR1 and a heavy chain FR4 having amino acid sequences of the FR1 and the FR4 contained in SEQ ID NO: 31. In some embodiments, the antibody provided herein comprises a heavy chain FR2 and a heavy chain FR3 having amino acid sequences of the FR2 and the FR3 contained in SEQ ID NO: 31. In some embodiments, the antibody provided herein comprises a heavy chain FR2 and a heavy chain FR4 having amino acid sequences of the FR2 and the FR4 contained in SEQ ID NO: 31. In some embodiments, the antibody provided herein comprises a heavy chain FR3 and a heavy chain FR4 having amino acid sequences of the FR3 and the FR4 contained in SEQ ID NO: 31.

In some embodiments, the antibody provided herein comprises a heavy chain FR1, a heavy chain FR2, and a heavy chain FR3 having amino acid sequences of the FR1, the FR2, and the FR3 contained in SEQ ID NO: 31. In some embodiments, the antibody provided herein comprises a heavy chain FR1, a heavy chain FR2, and a heavy chain FR4 having amino acid sequences of the FR1, the FR2, and the FR4 contained in SEQ ID NO: 31. In some embodiments, the antibody provided herein comprises a heavy chain FR1, a heavy chain FR3, and a heavy chain FR4 having amino acid sequences of the FR1, the FR3, and the FR4 contained in SEQ ID NO: 31. In some embodiments, the antibody provided herein comprises a heavy chain FR2, a heavy chain FR3, and a heavy chain FR4 having amino acid sequences of the FR2, the FR3, and the FR4 contained in SEQ ID NO: 31. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 31.

In some embodiments, the antibody provided herein further comprises one or more FR regions of the light chain FRs of 144L124B or 144L180A. In some embodiments, the antibody provided herein comprises a light chain FR1 having an amino acid sequence of the FR1 contained in SEQ ID NO: 55. In some embodiments, the antibody provided herein comprises a light chain FR2 having an amino acid sequence of the FR2 contained in SEQ ID NO: 55. In some embodiments, the antibody provided herein comprises a light chain FR3 having an amino acid sequence of the FR3 contained in SEQ ID NO: 55. In some embodiments, the antibody provided herein comprises a light chain FR4 having an amino acid sequence of the FR4 contained in SEQ ID NO: 55.

In some embodiments, the antibody provided herein comprises a light chain FR1 and a light chain FR2 having amino acid sequences of the FR1 and the FR2 contained in SEQ ID NO: 55. In some embodiments, the antibody provided herein comprises a light chain FR1 and a light chain FR3 having amino acid sequences of the FR1 and the FR3 contained in SEQ ID NO: 55. In some embodiments, the antibody provided herein comprises a light chain FR1 and a light chain FR4 having amino acid sequences of the FR1 and the FR4 contained in SEQ ID NO: 55. In some embodiments, the antibody provided herein comprises a light chain FR2 and a light chain FR3 having amino acid sequences of the FR2 and the FR3 contained in SEQ ID NO: 55. In some embodiments, the antibody provided herein comprises a light chain FR2 and a light chain FR4 having amino acid sequences of the FR2 and the FR4 contained in SEQ ID NO: 55. In some embodiments, the antibody provided herein comprises a light chain FR3 and a light chain FR4 having amino acid sequences of the FR3 and the FR4 contained in SEQ ID NO: 55.

In some embodiments, the antibody provided herein comprises a light chain FR1, a light chain FR2, and a light chain FR3 having amino acid sequences of the FR1, the FR2, and the FR3 contained in SEQ ID NO: 55. In some embodiments, the antibody provided herein comprises a light chain FR1, a light chain FR2, and a light chain FR4 having amino acid sequences of the FR1, the FR2, and the FR4 contained in SEQ ID NO: 55. In some embodiments, the antibody provided herein comprises a light chain FR1, a light chain FR3, and a light chain FR4 having amino acid sequences of the FR1, the FR3, and the FR4 contained in SEQ ID NO: 55. In some embodiments, the antibody provided herein comprises a light chain FR2, a light chain FR3, and a light chain FR4 having amino acid sequences of the FR2, the FR3, and the FR4 contained in SEQ ID NO: 55. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 55.

In some embodiments, the antibody provided herein further comprises one or more FR regions in the heavy chain of 144L133B. In some embodiments, the antibody provided herein comprises a heavy chain FR1 having an amino acid sequence of the FR1 contained in SEQ ID NO: 35. In some embodiments, the antibody provided herein comprises a heavy chain FR2 having an amino acid sequence of the FR2 contained in SEQ ID NO: 35. In some embodiments, the antibody provided herein comprises a heavy chain FR3 having an amino acid sequence of the FR3 contained in SEQ ID NO: 35. In some embodiments, the antibody provided herein comprises a heavy chain FR4 having an amino acid sequence of the FR4 contained in SEQ ID NO: 35.

In some embodiments, the antibody provided herein comprises a heavy chain FR1 and a heavy chain FR2 having amino acid sequences of the FR1 and the FR2 contained in SEQ ID NO: 35. In some embodiments, the antibody provided herein comprises a heavy chain FR1 and a heavy chain FR3 having amino acid sequences of the FR1 and the FR3 contained in SEQ ID NO: 35. In some embodiments, the antibody provided herein comprises a heavy chain FR1 and a heavy chain FR4 having amino acid sequences of the FR1 and the FR4 contained in SEQ ID NO: 35. In some embodiments, the antibody provided herein comprises a heavy chain FR2 and a heavy chain FR3 having amino acid sequences of the FR2 and the FR3 contained in SEQ ID NO: 35. In some embodiments, the antibody provided herein comprises a heavy chain FR2 and a heavy chain FR4 having amino acid sequences of the FR2 and the FR4 contained in SEQ ID NO: 35. In some embodiments, the antibody provided herein comprises a heavy chain FR3 and a heavy chain FR4 having amino acid sequences of the FR3 and the FR4 contained in SEQ ID NO: 35.

In some embodiments, the antibody provided herein comprises a heavy chain FR1, a heavy chain FR2, and a heavy chain FR3 having amino acid sequences of the FR1, the FR2, and the FR3 contained in SEQ ID NO: 35. In some embodiments, the antibody provided herein comprises a heavy chain FR1, a heavy chain FR2, and a heavy chain FR4 having amino acid sequences of the FR1, the FR2, and the FR4 contained in SEQ ID NO: 35. In some embodiments, the antibody provided herein comprises a heavy chain FR1, a heavy chain FR3, and a heavy chain FR4 having amino acid sequences of the FR1, the FR3, and the FR4 contained in SEQ ID NO: 35. In some embodiments, the antibody provided herein comprises a heavy chain FR2, a heavy chain FR3, and a heavy chain FR4 having amino acid sequences of the FR2, the FR3, and the FR4 contained in SEQ ID NO: 35. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 35.

In some embodiments, the antibody provided herein further comprises one or more FR regions of the light chain FRs of 144L133B. In some embodiments, the antibody provided herein comprises a light chain FR1 having an amino acid sequence of the FR1 contained in SEQ ID NO: 55. In some embodiments, the antibody provided herein comprises a light chain FR2 having an amino acid sequence of the FR2 contained in SEQ ID NO: 55. In some embodiments, the antibody provided herein comprises a light chain FR3 having an amino acid sequence of the FR3 contained in SEQ ID NO: 55. In some embodiments, the antibody provided herein comprises a light chain FR4 having an amino acid sequence of the FR4 contained in SEQ ID NO: 55.

In some embodiments, the antibody provided herein comprises a light chain FR1 and a light chain FR2 having amino acid sequences of the FR1 and the FR2 contained in SEQ ID NO: 55. In some embodiments, the antibody provided herein comprises a light chain FR1 and a light chain FR3 having amino acid sequences of the FR1 and the FR3 contained in SEQ ID NO: 55. In some embodiments, the antibody provided herein comprises a light chain FR1 and a light chain FR4 having amino acid sequences of the FR1 and the FR4 contained in SEQ ID NO: 55. In some embodiments, the antibody provided herein comprises a light chain FR2 and a light chain FR3 having amino acid sequences of the FR2 and the FR3 contained in SEQ ID NO: 55. In some embodiments, the antibody provided herein comprises a light chain FR2 and a light chain FR4 having amino acid sequences of the FR2 and the FR4 contained in SEQ ID NO: 55. In some embodiments, the antibody provided herein comprises a light chain FR3 and a light chain FR4 having amino acid sequences of the FR3 and the FR4 contained in SEQ ID NO: 55.

In some embodiments, the antibody provided herein comprises a light chain FR1, a light chain FR2, and a light chain FR3 having amino acid sequences of the FR1, the FR2, and the FR3 contained in SEQ ID NO: 55. In some embodiments, the antibody provided herein comprises a light chain FR1, a light chain FR2, and a light chain FR4 having amino acid sequences of the FR1, the FR2, and the FR4 contained in SEQ ID NO: 55. In some embodiments, the antibody provided herein comprises a light chain FR1, a light chain FR3, and a light chain FR4 having amino acid sequences of the FR1, the FR3, and the FR4 contained in SEQ ID NO: 55. In some embodiments, the antibody provided herein comprises a light chain FR2, a light chain FR3, and a light chain FR4 having amino acid sequences of the FR2, the FR3, and the FR4 contained in SEQ ID NO: 55. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 55.

In some embodiments, the antibody provided herein further comprises one or more FR regions in the heavy chain of 144L472A. In some embodiments, the antibody provided herein comprises a heavy chain FR1 having an amino acid sequence of the FR1 contained in SEQ ID NO: 39. In some embodiments, the antibody provided herein comprises a heavy chain FR2 having an amino acid sequence of the FR2 contained in SEQ ID NO: 39. In some embodiments, the antibody provided herein comprises a heavy chain FR3 having an amino acid sequence of the FR3 contained in SEQ ID NO: 39. In some embodiments, the antibody provided herein comprises a heavy chain FR4 having an amino acid sequence of the FR4 contained in SEQ ID NO: 39.

In some embodiments, the antibody provided herein comprises a heavy chain FR1 and a heavy chain FR2 having amino acid sequences of the FR1 and the FR2 contained in SEQ ID NO: 39. In some embodiments, the antibody provided herein comprises a heavy chain FR1 and a heavy chain FR3 having amino acid sequences of the FR1 and the FR3 contained in SEQ ID NO: 39. In some embodiments, the antibody provided herein comprises a heavy chain FR1 and a heavy chain FR4 having amino acid sequences of the FR1 and the FR4 contained in SEQ ID NO: 39. In some embodiments, the antibody provided herein comprises a heavy chain FR2 and a heavy chain FR3 having amino acid sequences of the FR2 and the FR3 contained in SEQ ID NO: 39. In some embodiments, the antibody provided herein comprises a heavy chain FR2 and a heavy chain FR4 having amino acid sequences of the FR2 and the FR4 contained in SEQ ID NO: 39. In some embodiments, the antibody provided herein comprises a heavy chain FR3 and a heavy chain FR4 having amino acid sequences of the FR3 and the FR4 contained in SEQ ID NO: 39.

In some embodiments, the antibody provided herein comprises a heavy chain FR1, a heavy chain FR2, and a heavy chain FR3 having amino acid sequences of the FR1, the FR2, and the FR3 contained in SEQ ID NO: 39. In some embodiments, the antibody provided herein comprises a heavy chain FR1, a heavy chain FR2, and a heavy chain FR4 having amino acid sequences of the FR1, the FR2, and the FR4 contained in SEQ ID NO: 39. In some embodiments, the antibody provided herein comprises a heavy chain FR1, a heavy chain FR3, and a heavy chain FR4 having amino acid sequences of the FR1, the FR3, and the FR4 contained in SEQ ID NO: 39. In some embodiments, the antibody provided herein comprises a heavy chain FR2, a heavy chain FR3, and a heavy chain FR4 having amino acid sequences of the FR2, the FR3, and the FR4 contained in SEQ ID NO: 39. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 39.

In some embodiments, the antibody provided herein further comprises one or more FR regions of the light chain FRs of 144L472A. In some embodiments, the antibody provided herein comprises a light chain FR1 having an amino acid sequence of the FR1 contained in SEQ ID NO: 59. In some embodiments, the antibody provided herein comprises a light chain FR2 having an amino acid sequence of the FR2 contained in SEQ ID NO: 59. In some embodiments, the antibody provided herein comprises a light chain FR3 having an amino acid sequence of the FR3 contained in SEQ ID NO: 59. In some embodiments, the antibody provided herein comprises a light chain FR4 having an amino acid sequence of the FR4 contained in SEQ ID NO: 59.

In some embodiments, the antibody provided herein comprises a light chain FR1 and a light chain FR2 having amino acid sequences of the FR1 and the FR2 contained in SEQ ID NO: 59. In some embodiments, the antibody provided herein comprises a light chain FR1 and a light chain FR3 having amino acid sequences of the FR1 and the FR3 contained in SEQ ID NO: 59. In some embodiments, the antibody provided herein comprises a light chain FR1 and a light chain FR4 having amino acid sequences of the FR1 and the FR4 contained in SEQ ID NO: 59. In some embodiments, the antibody provided herein comprises a light chain FR2 and a light chain FR3 having amino acid sequences of the FR2 and the FR3 contained in SEQ ID NO: 59. In some embodiments, the antibody provided herein comprises a light chain FR2 and a light chain FR4 having amino acid sequences of the FR2 and the FR4 contained in SEQ ID NO: 59. In some embodiments, the antibody provided herein comprises a light chain FR3 and a light chain FR4 having amino acid sequences of the FR3 and the FR4 contained in SEQ ID NO: 59.

In some embodiments, the antibody provided herein comprises a light chain FR1, a light chain FR2, and a light chain FR3 having amino acid sequences of the FR1, the FR2, and the FR3 contained in SEQ ID NO: 59. In some embodiments, the antibody provided herein comprises a light chain FR1, a light chain FR2, and a light chain FR4 having amino acid sequences of the FR1, the FR2, and the FR4 contained in SEQ ID NO: 59. In some embodiments, the antibody provided herein comprises a light chain FR1, a light chain FR3, and a light chain FR4 having amino acid sequences of the FR1, the FR3, and the FR4 contained in SEQ ID NO: 59. In some embodiments, the antibody provided herein comprises a light chain FR2, a light chain FR3, and a light chain FR4 having amino acid sequences of the FR2, the FR3, and the FR4 contained in SEQ ID NO: 59. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 59.

In some embodiments, the antibody provided herein further comprises one or more FR regions in the heavy chain of 144D666C. In some embodiments, the antibody provided herein comprises a heavy chain FR1 having an amino acid sequence of the FR1 contained in SEQ ID NO: 43. In some embodiments, the antibody provided herein comprises a heavy chain FR2 having an amino acid sequence of the FR2 contained in SEQ ID NO: 43. In some embodiments, the antibody provided herein comprises a heavy chain FR3 having an amino acid sequence of the FR3 contained in SEQ ID NO: 43. In some embodiments, the antibody provided herein comprises a heavy chain FR4 having an amino acid sequence of the FR4 contained in SEQ ID NO: 43.

In some embodiments, the antibody provided herein comprises a heavy chain FR1 and a heavy chain FR2 having amino acid sequences of the FR1 and the FR2 contained in SEQ ID NO: 43. In some embodiments, the antibody provided herein comprises a heavy chain FR1 and a heavy chain FR3 having amino acid sequences of the FR1 and the FR3 contained in SEQ ID NO: 43. In some embodiments, the antibody provided herein comprises a heavy chain FR1 and a heavy chain FR4 having amino acid sequences of the FR1 and the FR4 contained in SEQ ID NO: 43. In some embodiments, the antibody provided herein comprises a heavy chain FR2 and a heavy chain FR3 having amino acid sequences of the FR2 and the FR3 contained in SEQ ID NO: 43. In some embodiments, the antibody provided herein comprises a heavy chain FR2 and a heavy chain FR4 having amino acid sequences of the FR2 and the FR4 contained in SEQ ID NO: 43. In some embodiments, the antibody provided herein comprises a heavy chain FR3 and a heavy chain FR4 having amino acid sequences of the FR3 and the FR4 contained in SEQ ID NO: 43.

In some embodiments, the antibody provided herein comprises a heavy chain FR1, a heavy chain FR2, and a heavy chain FR3 having amino acid sequences of the FR1, the FR2, and the FR3 contained in SEQ ID NO: 43. In some embodiments, the antibody provided herein comprises a heavy chain FR1, a heavy chain FR2, and a heavy chain FR4 having amino acid sequences of the FR1, the FR2, and the FR4 contained in SEQ ID NO: 43. In some embodiments, the antibody provided herein comprises a heavy chain FR1, a heavy chain FR3, and a heavy chain FR4 having amino acid sequences of the FR1, the FR3, and the FR4 contained in SEQ ID NO: 43. In some embodiments, the antibody provided herein comprises a heavy chain FR2, a heavy chain FR3, and a heavy chain FR4 having amino acid sequences of the FR2, the FR3, and the FR4 contained in SEQ ID NO: 43. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 43.

In some embodiments, the antibody provided herein further comprises one or more FR regions of the light chain FRs of 144D666C. In some embodiments, the antibody provided herein comprises a light chain FR1 having an amino acid sequence of the FR1 contained in SEQ ID NO: 63. In some embodiments, the antibody provided herein comprises a light chain FR2 having an amino acid sequence of the FR2 contained in SEQ ID NO: 63. In some embodiments, the antibody provided herein comprises a light chain FR3 having an amino acid sequence of the FR3 contained in SEQ ID NO: 63. In some embodiments, the antibody provided herein comprises a light chain FR4 having an amino acid sequence of the FR4 contained in SEQ ID NO: 63.

In some embodiments, the antibody provided herein comprises a light chain FR1 and a light chain FR2 having amino acid sequences of the FR1 and the FR2 contained in SEQ ID NO: 63. In some embodiments, the antibody provided herein comprises a light chain FR1 and a light chain FR3 having amino acid sequences of the FR1 and the FR3 contained in SEQ ID NO: 63. In some embodiments, the antibody provided herein comprises a light chain FR1 and a light chain FR4 having amino acid sequences of the FR1 and the FR4 contained in SEQ ID NO: 63. In some embodiments, the antibody provided herein comprises a light chain FR2 and a light chain FR3 having amino acid sequences of the FR2 and the FR3 contained in SEQ ID NO: 63. In some embodiments, the antibody provided herein comprises a light chain FR2 and a light chain FR4 having amino acid sequences of the FR2 and the FR4 contained in SEQ ID NO: 63. In some embodiments, the antibody provided herein comprises a light chain FR3 and a light chain FR4 having amino acid sequences of the FR3 and the FR4 contained in SEQ ID NO: 63.

In some embodiments, the antibody provided herein comprises a light chain FR1, a light chain FR2, and a light chain FR3 having amino acid sequences of the FR1, the FR2, and the FR3 contained in SEQ ID NO: 63. In some embodiments, the antibody provided herein comprises a light chain FR1, a light chain FR2, and a light chain FR4 having amino acid sequences of the FR1, the FR2, and the FR4 contained in SEQ ID NO: 63. In some embodiments, the antibody provided herein comprises a light chain FR1, a light chain FR3, and a light chain FR4 having amino acid sequences of the FR1, the FR3, and the FR4 contained in SEQ ID NO: 63. In some embodiments, the antibody provided herein comprises a light chain FR2, a light chain FR3, and a light chain FR4 having amino acid sequences of the FR2, the FR3, and the FR4 contained in SEQ ID NO: 63. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 63.

In some embodiments, the antibody provided herein further comprises one or more FR regions in the heavy chain of 144J171G. In some embodiments, the antibody provided herein comprises a heavy chain FR1 having an amino acid sequence of the FR1 contained in SEQ ID NO: 47. In some embodiments, the antibody provided herein comprises a heavy chain FR2 having an amino acid sequence of the FR2 contained in SEQ ID NO: 47. In some embodiments, the antibody provided herein comprises a heavy chain FR3 having an amino acid sequence of the FR3 contained in SEQ ID NO: 47. In some embodiments, the antibody provided herein comprises a heavy chain FR4 having an amino acid sequence of the FR4 contained in SEQ ID NO: 47.

In some embodiments, the antibody provided herein comprises a heavy chain FR1 and a heavy chain FR2 having amino acid sequences of the FR1 and the FR2 contained in SEQ ID NO: 47. In some embodiments, the antibody provided herein comprises a heavy chain FR1 and a heavy chain FR3 having amino acid sequences of the FR1 and the FR3 contained in SEQ ID NO: 47. In some embodiments, the antibody provided herein comprises a heavy chain FR1 and a heavy chain FR4 having amino acid sequences of the FR1 and the FR4 contained in SEQ ID NO: 47. In some embodiments, the antibody provided herein comprises a heavy chain FR2 and a heavy chain FR3 having amino acid sequences of the FR2 and the FR3 contained in SEQ ID NO: 47. In some embodiments, the antibody provided herein comprises a heavy chain FR2 and a heavy chain FR4 having amino acid sequences of the FR2 and the FR4 contained in SEQ ID NO: 47. In some embodiments, the antibody provided herein comprises a heavy chain FR3 and a heavy chain FR4 having amino acid sequences of the FR3 and the FR4 contained in SEQ ID NO: 47.

In some embodiments, the antibody provided herein comprises a heavy chain FR1, a heavy chain FR2, and a heavy chain FR3 having amino acid sequences of the FR1, the FR2, and the FR3 contained in SEQ ID NO: 47. In some embodiments, the antibody provided herein comprises a heavy chain FR1, a heavy chain FR2, and a heavy chain FR4 having amino acid sequences of the FR1, the FR2, and the FR4 contained in SEQ ID NO: 47. In some embodiments, the antibody provided herein comprises a heavy chain FR1, a heavy chain FR3, and a heavy chain FR4 having amino acid sequences of the FR1, the FR3, and the FR4 contained in SEQ ID NO: 47. In some embodiments, the antibody provided herein comprises a heavy chain FR2, a heavy chain FR3, and a heavy chain FR4 having amino acid sequences of the FR2, the FR3, and the FR4 contained in SEQ ID NO: 47. In a specific embodiment, the antibody provided herein comprises a heavy chain FR1, a heavy chain FR2, a heavy chain FR3, and a heavy chain FR4 having amino acid sequences of the FR1, the FR2, the FR3, and the FR4 contained in SEQ ID NO: 47.

In some embodiments, the antibody provided herein further comprises one or more FR regions of the light chain FRs of 144J171G. In some embodiments, the antibody provided herein comprises a light chain FR1 having an amino acid sequence of the FR1 contained in SEQ ID NO: 67. In some embodiments, the antibody provided herein comprises a light chain FR2 having an amino acid sequence of the FR2 contained in SEQ ID NO: 67. In some embodiments, the antibody provided herein comprises a light chain FR3 having an amino acid sequence of the FR3 contained in SEQ ID NO: 67. In some embodiments, the antibody provided herein comprises a light chain FR4 having an amino acid sequence of the FR4 contained in SEQ ID NO: 67.

In some embodiments, the antibody provided herein comprises a light chain FR1 and a light chain FR2 having amino acid sequences of the FR1 and the FR2 contained in SEQ ID NO: 67. In some embodiments, the antibody provided herein comprises a light chain FR1 and a light chain FR3 having amino acid sequences of the FR1 and the FR3 contained in SEQ ID NO: 67. In some embodiments, the antibody provided herein comprises a light chain FR1 and a light chain FR4 having amino acid sequences of the FR1 and the FR4 contained in SEQ ID NO: 67. In some embodiments, the antibody provided herein comprises a light chain FR2 and a light chain FR3 having amino acid sequences of the FR2 and the FR3 contained in SEQ ID NO: 67. In some embodiments, the antibody provided herein comprises a light chain FR2 and a light chain FR4 having amino acid sequences of the FR2 and the FR4 contained in SEQ ID NO: 67. In some embodiments, the antibody provided herein comprises a light chain FR3 and a light chain FR4 having amino acid sequences of the FR3 and the FR4 contained in SEQ ID NO: 67.

In some embodiments, the antibody provided herein comprises a light chain FR1, a light chain FR2, and a light chain FR3 having amino acid sequences of the FR1, the FR2, and the FR3 contained in SEQ ID NO: 67. In some embodiments, the antibody provided herein comprises a light chain FR1, a light chain FR2, and a light chain FR4 having amino acid sequences of the FR1, the FR2, and the FR4 contained in SEQ ID NO: 67. In some embodiments, the antibody provided herein comprises a light chain FR1, a light chain FR3, and a light chain FR4 having amino acid sequences of the FR1, the FR3, and the FR4 contained in SEQ ID NO: 67. In some embodiments, the antibody provided herein comprises a light chain FR2, a light chain FR3, and a light chain FR4 having amino acid sequences of the FR2, the FR3, and the FR4 contained in SEQ ID NO: 67. In a specific embodiment, the antibody provided herein comprises a light chain FR1, a light chain FR2, a light chain FR3, and a light chain FR4 having amino acid sequences of the FR1, the FR2, the FR3, and the FR4 contained in SEQ ID NO: 67.

In some embodiments, the antibody or antigen binding fragment thereof provided herein comprises one or more FR regions of the hunaminzed antibodies described in Section 6 below and/or in FIGS. 10-13.

In some embodiments, the antibody or antigen binding fragment thereof comprises heavy chain FR regions contained in SEQ ID NOs: 115, 139-163, 165, and 171-177, and/or light chain FR regions contained in SEQ ID NOs: 114, 116-138, 164, and 166-170.

In some embodiments, the antibody or antigen binding fragment thereof comprises heavy chain FR regions contained in SEQ ID NOs: 115 and 139-163, and/or light chain FR regions contained in SEQ ID NOs: 114 and 116-138.

In some embodiments, the antibody or antigen binding fragment thereof comprises heavy chain FR regions contained in SEQ ID NO: 115 and light chain FR regions contained in SEQ ID NO: 114. In some embodiments, the antibody or antigen binding fragment thereof comprises heavy chain FR regions contained in SEQ ID NO: 115 and light chain FR regions contained in SEQ ID NO: 116. In some embodiments, the antibody or antigen binding fragment thereof comprises heavy chain FR regions contained in SEQ ID NO: 115 and light chain FR regions contained in SEQ ID NO: 117. In some embodiments, the antibody or antigen binding fragment thereof comprises heavy chain FR regions contained in SEQ ID NO: 115 and light chain FR regions contained in SEQ ID NO: 118. In some embodiments, the antibody or antigen binding fragment thereof comprises heavy chain FR regions contained in SEQ ID NO: 115 and light chain FR regions contained in SEQ ID NO: 119. In some embodiments, the antibody or antigen binding fragment thereof comprises heavy chain FR regions contained in SEQ ID NO: 115 and light chain FR regions contained in SEQ ID NO: 120. In some embodiments, the antibody or antigen binding fragment thereof comprises heavy chain FR regions contained in SEQ ID NO: 115 and light chain FR regions contained in SEQ ID NO: 121. In some embodiments, the antibody or antigen binding fragment thereof comprises heavy chain FR regions contained in SEQ ID NO: 115 and light chain FR regions contained in SEQ ID NO: 122. In some embodiments, the antibody or antigen binding fragment thereof comprises heavy chain FR regions contained in SEQ ID NO: 115 and light chain FR regions contained in SEQ ID NO: 123. In some embodiments, the antibody or antigen binding fragment thereof comprises heavy chain FR regions contained in SEQ ID NO: 115 and light chain FR regions contained in SEQ ID NO: 124. In some embodiments, the antibody or antigen binding fragment thereof comprises heavy chain FR regions contained in SEQ ID NO: 115 and light chain FR regions contained in SEQ ID NO: 125. In some embodiments, the antibody or antigen binding fragment thereof comprises heavy chain FR regions contained in SEQ ID NO: 115 and light chain FR regions contained in SEQ ID NO: 126. In some embodiments, the antibody or antigen binding fragment thereof comprises heavy chain FR regions contained in SEQ ID NO: 115 and light chain FR regions contained in SEQ ID NO: 127. In some embodiments, the antibody or antigen binding fragment thereof comprises heavy chain FR regions contained in SEQ ID NO: 115 and light chain FR regions contained in SEQ ID NO: 128. In some embodiments, the antibody or antigen binding fragment thereof comprises heavy chain FR regions contained in SEQ ID NO: 115 and light chain FR regions contained in SEQ ID NO: 129. In some embodiments, the antibody or antigen binding fragment thereof comprises heavy chain FR regions contained in SEQ ID NO: 115 and light chain FR regions contained in SEQ ID NO: 130. In some embodiments, the antibody or antigen binding fragment thereof comprises heavy chain FR regions contained in SEQ ID NO: 115 and light chain FR regions contained in SEQ ID NO: 131. In some embodiments, the antibody or antigen binding fragment thereof comprises heavy chain FR regions contained in SEQ ID NO: 115 and light chain FR regions contained in SEQ ID NO: 132. In some embodiments, the antibody or antigen binding fragment thereof comprises heavy chain FR regions contained in SEQ ID NO: 115 and light chain FR regions contained in SEQ ID NO: 133. In some embodiments, the antibody or antigen binding fragment thereof comprises heavy chain FR regions contained in SEQ ID NO: 115 and light chain FR regions contained in SEQ ID NO: 134. In some embodiments, the antibody or antigen binding fragment thereof comprises heavy chain FR regions contained in SEQ ID NO: 115 and light chain FR regions contained in SEQ ID NO: 135. In some embodiments, the antibody or antigen binding fragment thereof comprises heavy chain FR regions contained in SEQ ID NO: 115 and light chain FR regions contained in SEQ ID NO: 136. In some embodiments, the antibody or antigen binding fragment thereof comprises heavy chain FR regions contained in SEQ ID NO: 115 and light chain FR regions contained in SEQ ID NO: 137. In some embodiments, the antibody or antigen binding fragment thereof comprises heavy chain FR regions contained in SEQ ID NO: 115 and light chain FR regions contained in SEQ ID NO: 138.

In some embodiments, the antibody or antigen binding fragment thereof comprises heavy chain FR regions contained in SEQ ID NO: 139 and light chain FR regions contained in SEQ ID NO: 114. In some embodiments, the antibody or antigen binding fragment thereof comprises heavy chain FR regions contained in SEQ ID NO: 139 and light chain FR regions contained in SEQ ID NO: 116. In some embodiments, the antibody or antigen binding fragment thereof comprises heavy chain FR regions contained in SEQ ID NO: 139 and light chain FR regions contained in SEQ ID NO: 117. In some embodiments, the antibody or antigen binding fragment thereof comprises heavy chain FR regions contained in SEQ ID NO: 139 and light chain FR regions contained in SEQ ID NO: 118. In some embodiments, the antibody or antigen binding fragment thereof comprises heavy chain FR regions contained in SEQ ID NO: 139 and light chain FR regions contained in SEQ ID NO: 119. In some embodiments, the antibody or antigen binding fragment thereof comprises heavy chain FR regions contained in SEQ ID NO: 139 and light chain FR regions contained in SEQ ID NO: 120. In some embodiments, the antibody or antigen binding fragment thereof comprises heavy chain FR regions contained in SEQ ID NO: 139 and light chain FR regions contained in SEQ ID NO: 121. In some embodiments, the antibody or antigen binding fragment thereof comprises heavy chain FR regions contained in SEQ ID NO: 139 and light chain FR regions contained in SEQ ID NO: 122. In some embodiments, the antibody or antigen binding fragment thereof comprises heavy chain FR regions contained in SEQ ID NO: 139 and light chain FR regions contained in SEQ ID NO: 123. In some embodiments, the antibody or antigen binding fragment thereof comprises heavy chain FR regions contained in SEQ ID NO: 139 and light chain FR regions contained in SEQ ID NO: 124. In some embodiments, the antibody or antigen binding fragment thereof comprises heavy chain FR regions contained in SEQ ID NO: 139 and light chain FR regions contained in SEQ ID NO: 125. In some embodiments, the antibody or antigen binding fragment thereof comprises heavy chain FR regions contained in SEQ ID NO: 139 and light chain FR regions contained in SEQ ID NO: 126. In some embodiments, the antibody or antigen binding fragment thereof comprises heavy chain FR regions contained in SEQ ID NO: 139 and light chain FR regions contained in SEQ ID NO: 127. In some embodiments, the antibody or antigen binding fragment thereof comprises heavy chain FR regions contained in SEQ ID NO: 139 and light chain FR regions contained in SEQ ID NO: 128. In some embodiments, the antibody or antigen binding fragment thereof comprises heavy chain FR regions contained in SEQ ID NO: 139 and light chain FR regions contained in SEQ ID NO: 129. In some embodiments, the antibody or antigen binding fragment thereof comprises heavy chain FR regions contained in SEQ ID NO: 139 and light chain FR regions contained in SEQ ID NO: 130. In some embodiments, the antibody or antigen binding fragment thereof comprises heavy chain FR regions contained in SEQ ID NO: 139 and light chain FR regions contained in SEQ ID NO: 131. In some embodiments, the antibody or antigen binding fragment thereof comprises heavy chain FR regions contained in SEQ ID NO: 139 and light chain FR regions contained in SEQ ID NO: 132. In some embodiments, the antibody or antigen binding fragment thereof comprises heavy chain FR regions contained in SEQ ID NO: 139 and light chain FR regions contained in SEQ ID NO: 133. In some embodiments, the antibody or antigen binding fragment thereof comprises heavy chain FR regions contained in SEQ ID NO: 139 and light chain FR regions contained in SEQ ID NO: 134. In some embodiments, the antibody or antigen binding fragment thereof comprises heavy chain FR regions contained in SEQ ID NO: 139 and light chain FR regions contained in SEQ ID NO: 135. In some embodiments, the antibody or antigen binding fragment thereof comprises heavy chain FR regions contained in SEQ ID NO: 139 and light chain FR regions contained in SEQ ID NO: 136. In some embodiments, the antibody or antigen binding fragment thereof comprises heavy chain FR regions contained in SEQ ID NO: 139 and light chain FR regions contained in SEQ ID NO: 137. In some embodiments, the antibody or antigen binding fragment thereof comprises heavy chain FR regions contained in SEQ ID NO: 139 and light chain FR regions contained in SEQ ID NO: 138.

In some embodiments, the antibody or antigen binding fragment thereof comprises heavy chain FR regions contained in SEQ ID NO: 140 and light chain FR regions contained in SEQ ID NO: 114. In some embodiments, the antibody or antigen binding fragment thereof comprises heavy chain FR regions contained in SEQ ID NO: 140 and light chain FR regions contained in SEQ ID NO: 116. In some embodiments, the antibody or antigen binding fragment thereof comprises heavy chain FR regions contained in SEQ ID NO: 140 and light chain FR regions contained in SEQ ID NO: 117. In some embodiments, the antibody or antigen binding fragment thereof comprises heavy chain FR regions contained in SEQ ID NO: 140 and light chain FR regions contained in SEQ ID NO: 118. In some embodiments, the antibody or antigen binding fragment thereof comprises heavy chain FR regions contained in SEQ ID NO: 140 and light chain FR regions contained in SEQ ID NO: 119. In some embodiments, the antibody or antigen binding fragment thereof comprises heavy chain FR regions contained in SEQ ID NO: 140 and light chain FR regions contained in SEQ ID NO: 120. In some embodiments, the antibody or antigen binding fragment thereof comprises heavy chain FR regions contained in SEQ ID NO: 140 and light chain FR regions contained in SEQ ID NO: 121. In some embodiments, the antibody or antigen binding fragment thereof comprises heavy chain FR regions contained in SEQ ID NO: 140 and light chain FR regions contained in SEQ ID NO: 122. In some embodiments, the antibody or antigen binding fragment thereof comprises heavy chain FR regions contained in SEQ ID NO: 140 and light chain FR regions contained in SEQ ID NO: 123. In some embodiments, the antibody or antigen binding fragment thereof comprises heavy chain FR regions contained in SEQ ID NO: 140 and light chain FR regions contained in SEQ ID NO: 124. In some embodiments, the antibody or antigen binding fragment thereof comprises heavy chain FR regions contained in SEQ ID NO: 140 and light chain FR regions contained in SEQ ID NO: 125. In some embodiments, the antibody or antigen binding fragment thereof comprises heavy chain FR regions contained in SEQ ID NO: 140 and light chain FR regions contained in SEQ ID NO: 126. In some embodiments, the antibody or antigen binding fragment thereof comprises heavy chain FR regions contained in SEQ ID NO: 140 and light chain FR regions contained in SEQ ID NO: 127. In some embodiments, the antibody or antigen binding fragment thereof comprises heavy chain FR regions contained in SEQ ID NO: 140 and light chain FR regions contained in SEQ ID NO: 128. In some embodiments, the antibody or antigen binding fragment thereof comprises heavy chain FR regions contained in SEQ ID NO: 140 and light chain FR regions contained in SEQ ID NO: 129. In some embodiments, the antibody or antigen binding fragment thereof comprises heavy chain FR regions contained in SEQ ID NO: 140 and light chain FR regions contained in SEQ ID NO: 130. In some embodiments, the antibody or antigen binding fragment thereof comprises heavy chain FR regions contained in SEQ ID NO: 140 and light chain FR regions contained in SEQ ID NO: 131. In some embodiments, the antibody or antigen binding fragment thereof comprises heavy chain FR regions contained in SEQ ID NO: 140 and light chain FR regions contained in SEQ ID NO: 132. In some embodiments, the antibody or antigen binding fragment thereof comprises heavy chain FR regions contained in SEQ ID NO: 140 and light chain FR regions contained in SEQ ID NO: 133. In some embodiments, the antibody or antigen binding fragment thereof comprises heavy chain FR regions contained in SEQ ID NO: 140 and light chain FR regions contained in SEQ ID NO: 134. In some embodiments, the antibody or antigen binding fragment thereof comprises heavy chain FR regions contained in SEQ ID NO: 140 and light chain FR regions contained in SEQ ID NO: 135. In some embodiments, the antibody or antigen binding fragment thereof comprises heavy chain FR regions contained in SEQ ID NO: 140 and light chain FR regions contained in SEQ ID NO: 136. In some embodiments, the antibody or antigen binding fragment thereof comprises heavy chain FR regions contained in SEQ ID NO: 140 and light chain FR regions contained in SEQ ID NO: 137. In some embodiments, the antibody or antigen binding fragment thereof comprises heavy chain FR regions contained in SEQ ID NO: 140 and light chain FR regions contained in SEQ ID NO: 138.

In some embodiments, the antibody or antigen binding fragment thereof comprises heavy chain FR regions contained in SEQ ID NO: 141 and light chain FR regions contained in SEQ ID NO: 114. In some embodiments, the antibody or antigen binding fragment thereof comprises heavy chain FR regions contained in SEQ ID NO: 141 and light chain FR regions contained in SEQ ID NO: 116. In some embodiments, the antibody or antigen binding fragment thereof comprises heavy chain FR regions contained in SEQ ID NO: 141 and light chain FR regions contained in SEQ ID NO: 117. In some embodiments, the antibody or antigen binding fragment thereof comprises heavy chain FR regions contained in SEQ ID NO: 141 and light chain FR regions contained in SEQ ID NO: 118. In some embodiments, the antibody or antigen binding fragment thereof comprises heavy chain FR regions contained in SEQ ID NO: 141 and light chain FR regions contained in SEQ ID NO: 119. In some embodiments, the antibody or antigen binding fragment thereof comprises heavy chain FR regions contained in SEQ ID NO: 141 and light chain FR regions contained in SEQ ID NO: 120. In some embodiments, the antibody or antigen binding fragment thereof comprises heavy chain FR regions contained in SEQ ID NO: 141 and light chain FR regions contained in SEQ ID NO: 121. In some embodiments, the antibody or antigen binding fragment thereof comprises heavy chain FR regions contained in SEQ ID NO: 141 and light chain FR regions contained in SEQ ID NO: 122. In some embodiments, the antibody or antigen binding fragment thereof comprises heavy chain FR regions contained in SEQ ID NO: 141 and light chain FR regions contained in SEQ ID NO: 123. In some embodiments, the antibody or antigen binding fragment thereof comprises heavy chain FR regions contained in SEQ ID NO: 141 and light chain FR regions contained in SEQ ID NO: 124. In some embodiments, the antibody or antigen binding fragment thereof comprises heavy chain FR regions contained in SEQ ID NO: 141 and light chain FR regions contained in SEQ ID NO: 125. In some embodiments, the antibody or antigen binding fragment thereof comprises heavy chain FR regions contained in SEQ ID NO: 141 and light chain FR regions contained in SEQ ID NO: 126. In some embodiments, the antibody or antigen binding fragment thereof comprises heavy chain FR regions contained in SEQ ID NO: 141 and light chain FR regions contained in SEQ ID NO: 127. In some embodiments, the antibody or antigen binding fragment thereof comprises heavy chain FR regions contained in SEQ ID NO: 141 and light chain FR regions contained in SEQ ID NO: 128. In some embodiments, the antibody or antigen binding fragment thereof comprises heavy chain FR regions contained in SEQ ID NO: 141 and light chain FR regions contained in SEQ ID NO: 129. In some embodiments, the antibody or antigen binding fragment thereof comprises heavy chain FR regions contained in SEQ ID NO: 141 and light chain FR regions contained in SEQ ID NO: 130. In some embodiments, the antibody or antigen binding fragment thereof comprises heavy chain FR regions contained in SEQ ID NO: 141 and light chain FR regions contained in SEQ ID NO: 131. In some embodiments, the antibody or antigen binding fragment thereof comprises heavy chain FR regions contained in SEQ ID NO: 141 and light chain FR regions contained in SEQ ID NO: 132. In some embodiments, the antibody or antigen binding fragment thereof comprises heavy chain FR regions contained in SEQ ID NO: 141 and light chain FR regions contained in SEQ ID NO: 133. In some embodiments, the antibody or antigen binding fragment thereof comprises heavy chain FR regions contained in SEQ ID NO: 141 and light chain FR regions contained in SEQ ID NO: 134. In some embodiments, the antibody or antigen binding fragment thereof comprises heavy chain FR regions contained in SEQ ID NO: 141 and light chain FR regions contained in SEQ ID NO: 135. In some embodiments, the antibody or antigen binding fragment thereof comprises heavy chain FR regions contained in SEQ ID NO: 141 and light chain FR regions contained in SEQ ID NO: 136. In some embodiments, the antibody or antigen binding fragment thereof comprises heavy chain FR regions contained in SEQ ID NO: 141 and light chain FR regions contained in SEQ ID NO: 137. In some embodiments, the antibody or antigen binding fragment thereof comprises heavy chain FR regions contained in SEQ ID NO: 141 and light chain FR regions contained in SEQ ID NO: 138.

In some embodiments, the antibody or antigen binding fragment thereof comprises heavy chain FR regions contained in SEQ ID NO: 142 and light chain FR regions contained in SEQ ID NO: 114. In some embodiments, the antibody or antigen binding fragment thereof comprises heavy chain FR regions contained in SEQ ID NO: 142 and light chain FR regions contained in SEQ ID NO: 116. In some embodiments, the antibody or antigen binding fragment thereof comprises heavy chain FR regions contained in SEQ ID NO: 142 and light chain FR regions contained in SEQ ID NO: 117. In some embodiments, the antibody or antigen binding fragment thereof comprises heavy chain FR regions contained in SEQ ID NO: 142 and light chain FR regions contained in SEQ ID NO: 118. In some embodiments, the antibody or antigen binding fragment thereof comprises heavy chain FR regions contained in SEQ ID NO: 142 and light chain FR regions contained in SEQ ID NO: 119. In some embodiments, the antibody or antigen binding fragment thereof comprises heavy chain FR regions contained in SEQ ID NO: 142 and light chain FR regions contained in SEQ ID NO: 120. In some embodiments, the antibody or antigen binding fragment thereof comprises heavy chain FR regions contained in SEQ ID NO: 142 and light chain FR regions contained in SEQ ID NO: 121. In some embodiments, the antibody or antigen binding fragment thereof comprises heavy chain FR regions contained in SEQ ID NO: 142 and light chain FR regions contained in SEQ ID NO: 122. In some embodiments, the antibody or antigen binding fragment thereof comprises heavy chain FR regions contained in SEQ ID NO: 142 and light chain FR regions contained in SEQ ID NO: 123. In some embodiments, the antibody or antigen binding fragment thereof comprises heavy chain FR regions contained in SEQ ID NO: 142 and light chain FR regions contained in SEQ ID NO: 124. In some embodiments, the antibody or antigen binding fragment thereof comprises heavy chain FR regions contained in SEQ ID NO: 142 and light chain FR regions contained in SEQ ID NO: 125. In some embodiments, the antibody or antigen binding fragment thereof comprises heavy chain FR regions contained in SEQ ID NO: 142 and light chain FR regions contained in SEQ ID NO: 126. In some embodiments, the antibody or antigen binding fragment thereof comprises heavy chain FR regions contained in SEQ ID NO: 142 and light chain FR regions contained in SEQ ID NO: 127. In some embodiments, the antibody or antigen binding fragment thereof comprises heavy chain FR regions contained in SEQ ID NO: 142 and light chain FR regions contained in SEQ ID NO: 128. In some embodiments, the antibody or antigen binding fragment thereof comprises heavy chain FR regions contained in SEQ ID NO: 142 and light chain FR regions contained in SEQ ID NO: 129. In some embodiments, the antibody or antigen binding fragment thereof comprises heavy chain FR regions contained in SEQ ID NO: 142 and light chain FR regions contained in SEQ ID NO: 130. In some embodiments, the antibody or antigen binding fragment thereof comprises heavy chain FR regions contained in SEQ ID NO: 142 and light chain FR regions contained in SEQ ID NO: 131. In some embodiments, the antibody or antigen binding fragment thereof comprises heavy chain FR regions contained in SEQ ID NO: 142 and light chain FR regions contained in SEQ ID NO: 132. In some embodiments, the antibody or antigen binding fragment thereof comprises heavy chain FR regions contained in SEQ ID NO: 142 and light chain FR regions contained in SEQ ID NO: 133. In some embodiments, the antibody or antigen binding fragment thereof comprises heavy chain FR regions contained in SEQ ID NO: 142 and light chain FR regions contained in SEQ ID NO: 134. In some embodiments, the antibody or antigen binding fragment thereof comprises heavy chain FR regions contained in SEQ ID NO: 142 and light chain FR regions contained in SEQ ID NO: 135. In some embodiments, the antibody or antigen binding fragment thereof comprises heavy chain FR regions contained in SEQ ID NO: 142 and light chain FR regions contained in SEQ ID NO: 136. In some embodiments, the antibody or antigen binding fragment thereof comprises heavy chain FR regions contained in SEQ ID NO: 142 and light chain FR regions contained in SEQ ID NO: 137. In some embodiments, the antibody or antigen binding fragment thereof comprises heavy chain FR regions contained in SEQ ID NO: 142 and light chain FR regions contained in SEQ ID NO: 138.

In some embodiments, the antibody or antigen binding fragment thereof comprises heavy chain FR regions contained in SEQ ID NO: 143 and light chain FR regions contained in SEQ ID NO: 114. In some embodiments, the antibody or antigen binding fragment thereof comprises heavy chain FR regions contained in SEQ ID NO: 143 and light chain FR regions contained in SEQ ID NO: 116. In some embodiments, the antibody or antigen binding fragment thereof comprises heavy chain FR regions contained in SEQ ID NO: 143 and light chain FR regions contained in SEQ ID NO: 117. In some embodiments, the antibody or antigen binding fragment thereof comprises heavy chain FR regions contained in SEQ ID NO: 143 and light chain FR regions contained in SEQ ID NO: 118. In some embodiments, the antibody or antigen binding fragment thereof comprises heavy chain FR regions contained in SEQ ID NO: 143 and light chain FR regions contained in SEQ ID NO: 119. In some embodiments, the antibody or antigen binding fragment thereof comprises heavy chain FR regions contained in SEQ ID NO: 143 and light chain FR regions contained in SEQ ID NO: 120. In some embodiments, the antibody or antigen binding fragment thereof comprises heavy chain FR regions contained in SEQ ID NO: 143 and light chain FR regions contained in SEQ ID NO: 121. In some embodiments, the antibody or antigen binding fragment thereof comprises heavy chain FR regions contained in SEQ ID NO: 143 and light chain FR regions contained in SEQ ID NO: 122. In some embodiments, the antibody or antigen binding fragment thereof comprises heavy chain FR regions contained in SEQ ID NO: 143 and light chain FR regions contained in SEQ ID NO: 123. In some embodiments, the antibody or antigen binding fragment thereof comprises heavy chain FR regions contained in SEQ ID NO: 143 and light chain FR regions contained in SEQ ID NO: 124. In some embodiments, the antibody or antigen binding fragment thereof comprises heavy chain FR regions contained in SEQ ID NO: 143 and light chain FR regions contained in SEQ ID NO: 125. In some embodiments, the antibody or antigen binding fragment thereof comprises heavy chain FR regions contained in SEQ ID NO: 143 and light chain FR regions contained in SEQ ID NO: 126. In some embodiments, the antibody or antigen binding fragment thereof comprises heavy chain FR regions contained in SEQ ID NO: 143 and light chain FR regions contained in SEQ ID NO: 127. In some embodiments, the antibody or antigen binding fragment thereof comprises heavy chain FR regions contained in SEQ ID NO: 143 and light chain FR regions contained in SEQ ID NO: 128. In some embodiments, the antibody or antigen binding fragment thereof comprises heavy chain FR regions contained in SEQ ID NO: 143 and light chain FR regions contained in SEQ ID NO: 129. In some embodiments, the antibody or antigen binding fragment thereof comprises heavy chain FR regions contained in SEQ ID NO: 143 and light chain FR regions contained in SEQ ID NO: 130. In some embodiments, the antibody or antigen binding fragment thereof comprises heavy chain FR regions contained in SEQ ID NO: 143 and light chain FR regions contained in SEQ ID NO: 131. In some embodiments, the antibody or antigen binding fragment thereof comprises heavy chain FR regions contained in SEQ ID NO: 143 and light chain FR regions contained in SEQ ID NO: 132. In some embodiments, the antibody or antigen binding fragment thereof comprises heavy chain FR regions contained in SEQ ID NO: 143 and light chain FR regions contained in SEQ ID NO: 133. In some embodiments, the antibody or antigen binding fragment thereof comprises heavy chain FR regions contained in SEQ ID NO: 143 and light chain FR regions contained in SEQ ID NO: 134. In some embodiments, the antibody or antigen binding fragment thereof comprises heavy chain FR regions contained in SEQ ID NO: 143 and light chain FR regions contained in SEQ ID NO: 135. In some embodiments, the antibody or antigen binding fragment thereof comprises heavy chain FR regions contained in SEQ ID NO: 143 and light chain FR regions contained in SEQ ID NO: 136. In some embodiments, the antibody or antigen binding fragment thereof comprises heavy chain FR regions contained in SEQ ID NO: 143 and light chain FR regions contained in SEQ ID NO: 137. In some embodiments, the antibody or antigen binding fragment thereof comprises heavy chain FR regions contained in SEQ ID NO: 143 and light chain FR regions contained in SEQ ID NO: 138.

In some embodiments, the antibody or antigen binding fragment thereof comprises heavy chain FR regions contained in SEQ ID NO: 144 and light chain FR regions contained in SEQ ID NO: 114. In some embodiments, the antibody or antigen binding fragment thereof comprises heavy chain FR regions contained in SEQ ID NO: 144 and light chain FR regions contained in SEQ ID NO: 116. In some embodiments, the antibody or antigen binding fragment thereof comprises heavy chain FR regions contained in SEQ ID NO: 144 and light chain FR regions contained in SEQ ID NO: 117. In some embodiments, the antibody or antigen binding fragment thereof comprises heavy chain FR regions contained in SEQ ID NO: 144 and light chain FR regions contained in SEQ ID NO: 118. In some embodiments, the antibody or antigen binding fragment thereof comprises heavy chain FR regions contained in SEQ ID NO: 144 and light chain FR regions contained in SEQ ID NO: 119. In some embodiments, the antibody or antigen binding fragment thereof comprises heavy chain FR regions contained in SEQ ID NO: 144 and light chain FR regions contained in SEQ ID NO: 120. In some embodiments, the antibody or antigen binding fragment thereof comprises heavy chain FR regions contained in SEQ ID NO: 144 and light chain FR regions contained in SEQ ID NO: 121. In some embodiments, the antibody or antigen binding fragment thereof comprises heavy chain FR regions contained in SEQ ID NO: 144 and light chain FR regions contained in SEQ ID NO: 122. In some embodiments, the antibody or antigen binding fragment thereof comprises heavy chain FR regions contained in SEQ ID NO: 144 and light chain FR regions contained in SEQ ID NO: 123. In some embodiments, the antibody or antigen binding fragment thereof comprises heavy chain FR regions contained in SEQ ID NO: 144 and light chain FR regions contained in SEQ ID NO: 124. In some embodiments, the antibody or antigen binding fragment thereof comprises heavy chain FR regions contained in SEQ ID NO: 144 and light chain FR regions contained in SEQ ID NO: 125. In some embodiments, the antibody or antigen binding fragment thereof comprises heavy chain FR regions contained in SEQ ID NO: 144 and light chain FR regions contained in SEQ ID NO: 126. In some embodiments, the antibody or antigen binding fragment thereof comprises heavy chain FR regions contained in SEQ ID NO: 144 and light chain FR regions contained in SEQ ID NO: 127. In some embodiments, the antibody or antigen binding fragment thereof comprises heavy chain FR regions contained in SEQ ID NO: 144 and light chain FR regions contained in SEQ ID NO: 128. In some embodiments, the antibody or antigen binding fragment thereof comprises heavy chain FR regions contained in SEQ ID NO: 144 and light chain FR regions contained in SEQ ID NO: 129. In some embodiments, the antibody or antigen binding fragment thereof comprises heavy chain FR regions contained in SEQ ID NO: 144 and light chain FR regions contained in SEQ ID NO: 130. In some embodiments, the antibody or antigen binding fragment thereof comprises heavy chain FR regions contained in SEQ ID NO: 144 and light chain FR regions contained in SEQ ID NO: 131. In some embodiments, the antibody or antigen binding fragment thereof comprises heavy chain FR regions contained in SEQ ID NO: 144 and light chain FR regions contained in SEQ ID NO: 132. In some embodiments, the antibody or antigen binding fragment thereof comprises heavy chain FR regions contained in SEQ ID NO: 144 and light chain FR regions contained in SEQ ID NO: 133. In some embodiments, the antibody or antigen binding fragment thereof comprises heavy chain FR regions contained in SEQ ID NO: 144 and light chain FR regions contained in SEQ ID NO: 134. In some embodiments, the antibody or antigen binding fragment thereof comprises heavy chain FR regions contained in SEQ ID NO: 144 and light chain FR regions contained in SEQ ID NO: 135. In some embodiments, the antibody or antigen binding fragment thereof comprises heavy chain FR regions contained in SEQ ID NO: 144 and light chain FR regions contained in SEQ ID NO: 136. In some embodiments, the antibody or antigen binding fragment thereof comprises heavy chain FR regions contained in SEQ ID NO: 144 and light chain FR regions contained in SEQ ID NO: 137. In some embodiments, the antibody or antigen binding fragment thereof comprises heavy chain FR regions contained in SEQ ID NO: 144 and light chain FR regions contained in SEQ ID NO: 138.

In some embodiments, the antibody or antigen binding fragment thereof comprises heavy chain FR regions contained in SEQ ID NO: 145 and light chain FR regions contained in SEQ ID NO: 114. In some embodiments, the antibody or antigen binding fragment thereof comprises heavy chain FR regions contained in SEQ ID NO: 145 and light chain FR regions contained in SEQ ID NO: 116. In some embodiments, the antibody or antigen binding fragment thereof comprises heavy chain FR regions contained in SEQ ID NO: 145 and light chain FR regions contained in SEQ ID NO: 117. In some embodiments, the antibody or antigen binding fragment thereof comprises heavy chain FR regions contained in SEQ ID NO: 145 and light chain FR regions contained in SEQ ID NO: 118. In some embodiments, the antibody or antigen binding fragment thereof comprises heavy chain FR regions contained in SEQ ID NO: 145 and light chain FR regions contained in SEQ ID NO: 119. In some embodiments, the antibody or antigen binding fragment thereof comprises heavy chain FR regions contained in SEQ ID NO: 145 and light chain FR regions contained in SEQ ID NO: 120. In some embodiments, the antibody or antigen binding fragment thereof comprises heavy chain FR regions contained in SEQ ID NO: 145 and light chain FR regions contained in SEQ ID NO: 121. In some embodiments, the antibody or antigen binding fragment thereof comprises heavy chain FR regions contained in SEQ ID NO: 145 and light chain FR regions contained in SEQ ID NO: 122. In some embodiments, the antibody or antigen binding fragment thereof comprises heavy chain FR regions contained in SEQ ID NO: 145 and light chain FR regions contained in SEQ ID NO: 123. In some embodiments, the antibody or antigen binding fragment thereof comprises heavy chain FR regions contained in SEQ ID NO: 145 and light chain FR regions contained in SEQ ID NO: 124. In some embodiments, the antibody or antigen binding fragment thereof comprises heavy chain FR regions contained in SEQ ID NO: 145 and light chain FR regions contained in SEQ ID NO: 125. In some embodiments, the antibody or antigen binding fragment thereof comprises heavy chain FR regions contained in SEQ ID NO: 145 and light chain FR regions contained in SEQ ID NO: 126. In some embodiments, the antibody or antigen binding fragment thereof comprises heavy chain FR regions contained in SEQ ID NO: 145 and light chain FR regions contained in SEQ ID NO: 127. In some embodiments, the antibody or antigen binding fragment thereof comprises heavy chain FR regions contained in SEQ ID NO: 145 and light chain FR regions contained in SEQ ID NO: 128. In some embodiments, the antibody or antigen binding fragment thereof comprises heavy chain FR regions contained in SEQ ID NO: 145 and light chain FR regions contained in SEQ ID NO: 129. In some embodiments, the antibody or antigen binding fragment thereof comprises heavy chain FR regions contained in SEQ ID NO: 145 and light chain FR regions contained in SEQ ID NO: 130. In some embodiments, the antibody or antigen binding fragment thereof comprises heavy chain FR regions contained in SEQ ID NO: 145 and light chain FR regions contained in SEQ ID NO: 131. In some embodiments, the antibody or antigen binding fragment thereof comprises heavy chain FR regions contained in SEQ ID NO: 145 and light chain FR regions contained in SEQ ID NO: 132. In some embodiments, the antibody or antigen binding fragment thereof comprises heavy chain FR regions contained in SEQ ID NO: 145 and light chain FR regions contained in SEQ ID NO: 133. In some embodiments, the antibody or antigen binding fragment thereof comprises heavy chain FR regions contained in SEQ ID NO: 145 and light chain FR regions contained in SEQ ID NO: 134. In some embodiments, the antibody or antigen binding fragment thereof comprises heavy chain FR regions contained in SEQ ID NO: 145 and light chain FR regions contained in SEQ ID NO: 135. In some embodiments, the antibody or antigen binding fragment thereof comprises heavy chain FR regions contained in SEQ ID NO: 145 and light chain FR regions contained in SEQ ID NO: 136. In some embodiments, the antibody or antigen binding fragment thereof comprises heavy chain FR regions contained in SEQ ID NO: 145 and light chain FR regions contained in SEQ ID NO: 137. In some embodiments, the antibody or antigen binding fragment thereof comprises heavy chain FR regions contained in SEQ ID NO: 145 and light chain FR regions contained in SEQ ID NO: 138.

In some embodiments, the antibody or antigen binding fragment thereof comprises heavy chain FR regions contained in SEQ ID NO: 146 and light chain FR regions contained in SEQ ID NO: 114. In some embodiments, the antibody or antigen binding fragment thereof comprises heavy chain FR regions contained in SEQ ID NO: 146 and light chain FR regions contained in SEQ ID NO: 116. In some embodiments, the antibody or antigen binding fragment thereof comprises heavy chain FR regions contained in SEQ ID NO: 146 and light chain FR regions contained in SEQ ID NO: 117. In some embodiments, the antibody or antigen binding fragment thereof comprises heavy chain FR regions contained in SEQ ID NO: 146 and light chain FR regions contained in SEQ ID NO: 118. In some embodiments, the antibody or antigen binding fragment thereof comprises heavy chain FR regions contained in SEQ ID NO: 146 and light chain FR regions contained in SEQ ID NO: 119. In some embodiments, the antibody or antigen binding fragment thereof comprises heavy chain FR regions contained in SEQ ID NO: 146 and light chain FR regions contained in SEQ ID NO: 120. In some embodiments, the antibody or antigen binding fragment thereof comprises heavy chain FR regions contained in SEQ ID NO: 146 and light chain FR regions contained in SEQ ID NO: 121. In some embodiments, the antibody or antigen binding fragment thereof comprises heavy chain FR regions contained in SEQ ID NO: 146 and light chain FR regions contained in SEQ ID NO: 122. In some embodiments, the antibody or antigen binding fragment thereof comprises heavy chain FR regions contained in SEQ ID NO: 146 and light chain FR regions contained in SEQ ID NO: 123. In some embodiments, the antibody or antigen binding fragment thereof comprises heavy chain FR regions contained in SEQ ID NO: 146 and light chain FR regions contained in SEQ ID NO: 124. In some embodiments, the antibody or antigen binding fragment thereof comprises heavy chain FR regions contained in SEQ ID NO: 146 and light chain FR regions contained in SEQ ID NO: 125. In some embodiments, the antibody or antigen binding fragment thereof comprises heavy chain FR regions contained in SEQ ID NO: 146 and light chain FR regions contained in SEQ ID NO: 126. In some embodiments, the antibody or antigen binding fragment thereof comprises heavy chain FR regions contained in SEQ ID NO: 146 and light chain FR regions contained in SEQ ID NO: 127. In some embodiments, the antibody or antigen binding fragment thereof comprises heavy chain FR regions contained in SEQ ID NO: 146 and light chain FR regions contained in SEQ ID NO: 128. In some embodiments, the antibody or antigen binding fragment thereof comprises heavy chain FR regions contained in SEQ ID NO: 146 and light chain FR regions contained in SEQ ID NO: 129. In some embodiments, the antibody or antigen binding fragment thereof comprises heavy chain FR regions contained in SEQ ID NO: 146 and light chain FR regions contained in SEQ ID NO: 130. In some embodiments, the antibody or antigen binding fragment thereof comprises heavy chain FR regions contained in SEQ ID NO: 146 and light chain FR regions contained in SEQ ID NO: 131. In some embodiments, the antibody or antigen binding fragment thereof comprises heavy chain FR regions contained in SEQ ID NO: 146 and light chain FR regions contained in SEQ ID NO: 132. In some embodiments, the antibody or antigen binding fragment thereof comprises heavy chain FR regions contained in SEQ ID NO: 146 and light chain FR regions contained in SEQ ID NO: 133. In some embodiments, the antibody or antigen binding fragment thereof comprises heavy chain FR regions contained in SEQ ID NO: 146 and light chain FR regions contained in SEQ ID NO: 134. In some embodiments, the antibody or antigen binding fragment thereof comprises heavy chain FR regions contained in SEQ ID NO: 146 and light chain FR regions contained in SEQ ID NO: 135. In some embodiments, the antibody or antigen binding fragment thereof comprises heavy chain FR regions contained in SEQ ID NO: 146 and light chain FR regions contained in SEQ ID NO: 136. In some embodiments, the antibody or antigen binding fragment thereof comprises heavy chain FR regions contained in SEQ ID NO: 146 and light chain FR regions contained in SEQ ID NO: 137. In some embodiments, the antibody or antigen binding fragment thereof comprises heavy chain FR regions contained in SEQ ID NO: 146 and light chain FR regions contained in SEQ ID NO: 138.

In some embodiments, the antibody or antigen binding fragment thereof comprises heavy chain FR regions contained in SEQ ID NO: 147 and light chain FR regions contained in SEQ ID NO: 114. In some embodiments, the antibody or antigen binding fragment thereof comprises heavy chain FR regions contained in SEQ ID NO: 147 and light chain FR regions contained in SEQ ID NO: 116. In some embodiments, the antibody or antigen binding fragment thereof comprises heavy chain FR regions contained in SEQ ID NO: 147 and light chain FR regions contained in SEQ ID NO: 117. In some embodiments, the antibody or antigen binding fragment thereof comprises heavy chain FR regions contained in SEQ ID NO: 147 and light chain FR regions contained in SEQ ID NO: 118. In some embodiments, the antibody or antigen binding fragment thereof comprises heavy chain FR regions contained in SEQ ID NO: 147 and light chain FR regions contained in SEQ ID NO: 119. In some embodiments, the antibody or antigen binding fragment thereof comprises heavy chain FR regions contained in SEQ ID NO: 147 and light chain FR regions contained in SEQ ID NO: 120. In some embodiments, the antibody or antigen binding fragment thereof comprises heavy chain FR regions contained in SEQ ID NO: 147 and light chain FR regions contained in SEQ ID NO: 121. In some embodiments, the antibody or antigen binding fragment thereof comprises heavy chain FR regions contained in SEQ ID NO: 147 and light chain FR regions contained in SEQ ID NO: 122. In some embodiments, the antibody or antigen binding fragment thereof comprises heavy chain FR regions contained in SEQ ID NO: 147 and light chain FR regions contained in SEQ ID NO: 123. In some embodiments, the antibody or antigen binding fragment thereof comprises heavy chain FR regions contained in SEQ ID NO: 147 and light chain FR regions contained in SEQ ID NO: 124. In some embodiments, the antibody or antigen binding fragment thereof comprises heavy chain FR regions contained in SEQ ID NO: 147 and light chain FR regions contained in SEQ ID NO: 125. In some embodiments, the antibody or antigen binding fragment thereof comprises heavy chain FR regions contained in SEQ ID NO: 147 and light chain FR regions contained in SEQ ID NO: 126. In some embodiments, the antibody or antigen binding fragment thereof comprises heavy chain FR regions contained in SEQ ID NO: 147 and light chain FR regions contained in SEQ ID NO: 127. In some embodiments, the antibody or antigen binding fragment thereof comprises heavy chain FR regions contained in SEQ ID NO: 147 and light chain FR regions contained in SEQ ID NO: 128. In some embodiments, the antibody or antigen binding fragment thereof comprises heavy chain FR regions contained in SEQ ID NO: 147 and light chain FR regions contained in SEQ ID NO: 129. In some embodiments, the antibody or antigen binding fragment thereof comprises heavy chain FR regions contained in SEQ ID NO: 147 and light chain FR regions contained in SEQ ID NO: 130. In some embodiments, the antibody or antigen binding fragment thereof comprises heavy chain FR regions contained in SEQ ID NO: 147 and light chain FR regions contained in SEQ ID NO: 131. In some embodiments, the antibody or antigen binding fragment thereof comprises heavy chain FR regions contained in SEQ ID NO: 147 and light chain FR regions contained in SEQ ID NO: 132. In some embodiments, the antibody or antigen binding fragment thereof comprises heavy chain FR regions contained in SEQ ID NO: 147 and light chain FR regions contained in SEQ ID NO: 133. In some embodiments, the antibody or antigen binding fragment thereof comprises heavy chain FR regions contained in SEQ ID NO: 147 and light chain FR regions contained in SEQ ID NO: 134. In some embodiments, the antibody or antigen binding fragment thereof comprises heavy chain FR regions contained in SEQ ID NO: 147 and light chain FR regions contained in SEQ ID NO: 135. In some embodiments, the antibody or antigen binding fragment thereof comprises heavy chain FR regions contained in SEQ ID NO: 147 and light chain FR regions contained in SEQ ID NO: 136. In some embodiments, the antibody or antigen binding fragment thereof comprises heavy chain FR regions contained in SEQ ID NO: 147 and light chain FR regions contained in SEQ ID NO: 137. In some embodiments, the antibody or antigen binding fragment thereof comprises heavy chain FR regions contained in SEQ ID NO: 147 and light chain FR regions contained in SEQ ID NO: 138.

In some embodiments, the antibody or antigen binding fragment thereof comprises heavy chain FR regions contained in SEQ ID NO: 148 and light chain FR regions contained in SEQ ID NO: 114. In some embodiments, the antibody or antigen binding fragment thereof comprises heavy chain FR regions contained in SEQ ID NO: 148 and light chain FR regions contained in SEQ ID NO: 116. In some embodiments, the antibody or antigen binding fragment thereof comprises heavy chain FR regions contained in SEQ ID NO: 148 and light chain FR regions contained in SEQ ID NO: 117. In some embodiments, the antibody or antigen binding fragment thereof comprises heavy chain FR regions contained in SEQ ID NO: 148 and light chain FR regions contained in SEQ ID NO: 118. In some embodiments, the antibody or antigen binding fragment thereof comprises heavy chain FR regions contained in SEQ ID NO: 148 and light chain FR regions contained in SEQ ID NO: 119. In some embodiments, the antibody or antigen binding fragment thereof comprises heavy chain FR regions contained in SEQ ID NO: 148 and light chain FR regions contained in SEQ ID NO: 120. In some embodiments, the antibody or antigen binding fragment thereof comprises heavy chain FR regions contained in SEQ ID NO: 148 and light chain FR regions contained in SEQ ID NO: 121. In some embodiments, the antibody or antigen binding fragment thereof comprises heavy chain FR regions contained in SEQ ID NO: 148 and light chain FR regions contained in SEQ ID NO: 122. In some embodiments, the antibody or antigen binding fragment thereof comprises heavy chain FR regions contained in SEQ ID NO: 148 and light chain FR regions contained in SEQ ID NO: 123. In some embodiments, the antibody or antigen binding fragment thereof comprises heavy chain FR regions contained in SEQ ID NO: 148 and light chain FR regions contained in SEQ ID NO: 124. In some embodiments, the antibody or antigen binding fragment thereof comprises heavy chain FR regions contained in SEQ ID NO: 148 and light chain FR regions contained in SEQ ID NO: 125. In some embodiments, the antibody or antigen binding fragment thereof comprises heavy chain FR regions contained in SEQ ID NO: 148 and light chain FR regions contained in SEQ ID NO: 126. In some embodiments, the antibody or antigen binding fragment thereof comprises heavy chain FR regions contained in SEQ ID NO: 148 and light chain FR regions contained in SEQ ID NO: 127. In some embodiments, the antibody or antigen binding fragment thereof comprises heavy chain FR regions contained in SEQ ID NO: 148 and light chain FR regions contained in SEQ ID NO: 128. In some embodiments, the antibody or antigen binding fragment thereof comprises heavy chain FR regions contained in SEQ ID NO: 148 and light chain FR regions contained in SEQ ID NO: 129. In some embodiments, the antibody or antigen binding fragment thereof comprises heavy chain FR regions contained in SEQ ID NO: 148 and light chain FR regions contained in SEQ ID NO: 130. In some embodiments, the antibody or antigen binding fragment thereof comprises heavy chain FR regions contained in SEQ ID NO: 148 and light chain FR regions contained in SEQ ID NO: 131. In some embodiments, the antibody or antigen binding fragment thereof comprises heavy chain FR regions contained in SEQ ID NO: 148 and light chain FR regions contained in SEQ ID NO: 132. In some embodiments, the antibody or antigen binding fragment thereof comprises heavy chain FR regions contained in SEQ ID NO: 148 and light chain FR regions contained in SEQ ID NO: 133. In some embodiments, the antibody or antigen binding fragment thereof comprises heavy chain FR regions contained in SEQ ID NO: 148 and light chain FR regions contained in SEQ ID NO: 134. In some embodiments, the antibody or antigen binding fragment thereof comprises heavy chain FR regions contained in SEQ ID NO: 148 and light chain FR regions contained in SEQ ID NO: 135. In some embodiments, the antibody or antigen binding fragment thereof comprises heavy chain FR regions contained in SEQ ID NO: 148 and light chain FR regions contained in SEQ ID NO: 136. In some embodiments, the antibody or antigen binding fragment thereof comprises heavy chain FR regions contained in SEQ ID NO: 148 and light chain FR regions contained in SEQ ID NO: 137. In some embodiments, the antibody or antigen binding fragment thereof comprises heavy chain FR regions contained in SEQ ID NO: 148 and light chain FR regions contained in SEQ ID NO: 138.

In some embodiments, the antibody or antigen binding fragment thereof comprises heavy chain FR regions contained in SEQ ID NO: 149 and light chain FR regions contained in SEQ ID NO: 114. In some embodiments, the antibody or antigen binding fragment thereof comprises heavy chain FR regions contained in SEQ ID NO: 149 and light chain FR regions contained in SEQ ID NO: 116. In some embodiments, the antibody or antigen binding fragment thereof comprises heavy chain FR regions contained in SEQ ID NO: 149 and light chain FR regions contained in SEQ ID NO: 117. In some embodiments, the antibody or antigen binding fragment thereof comprises heavy chain FR regions contained in SEQ ID NO: 149 and light chain FR regions contained in SEQ ID NO: 118. In some embodiments, the antibody or antigen binding fragment thereof comprises heavy chain FR regions contained in SEQ ID NO: 149 and light chain FR regions contained in SEQ ID NO: 119. In some embodiments, the antibody or antigen binding fragment thereof comprises heavy chain FR regions contained in SEQ ID NO: 149 and light chain FR regions contained in SEQ ID NO: 120. In some embodiments, the antibody or antigen binding fragment thereof comprises heavy chain FR regions contained in SEQ ID NO: 149 and light chain FR regions contained in SEQ ID NO: 121. In some embodiments, the antibody or antigen binding fragment thereof comprises heavy chain FR regions contained in SEQ ID NO: 149 and light chain FR regions contained in SEQ ID NO: 122. In some embodiments, the antibody or antigen binding fragment thereof comprises heavy chain FR regions contained in SEQ ID NO: 149 and light chain FR regions contained in SEQ ID NO: 123. In some embodiments, the antibody or antigen binding fragment thereof comprises heavy chain FR regions contained in SEQ ID NO: 149 and light chain FR regions contained in SEQ ID NO: 124. In some embodiments, the antibody or antigen binding fragment thereof comprises heavy chain FR regions contained in SEQ ID NO: 149 and light chain FR regions contained in SEQ ID NO: 125. In some embodiments, the antibody or antigen binding fragment thereof comprises heavy chain FR regions contained in SEQ ID NO: 149 and light chain FR regions contained in SEQ ID NO: 126. In some embodiments, the antibody or antigen binding fragment thereof comprises heavy chain FR regions contained in SEQ ID NO: 149 and light chain FR regions contained in SEQ ID NO: 127. In some embodiments, the antibody or antigen binding fragment thereof comprises heavy chain FR regions contained in SEQ ID NO: 149 and light chain FR regions contained in SEQ ID NO: 128. In some embodiments, the antibody or antigen binding fragment thereof comprises heavy chain FR regions contained in SEQ ID NO: 149 and light chain FR regions contained in SEQ ID NO: 129. In some embodiments, the antibody or antigen binding fragment thereof comprises heavy chain FR regions contained in SEQ ID NO: 149 and light chain FR regions contained in SEQ ID NO: 130. In some embodiments, the antibody or antigen binding fragment thereof comprises heavy chain FR regions contained in SEQ ID NO: 149 and light chain FR regions contained in SEQ ID NO: 131. In some embodiments, the antibody or antigen binding fragment thereof comprises heavy chain FR regions contained in SEQ ID NO: 149 and light chain FR regions contained in SEQ ID NO: 132. In some embodiments, the antibody or antigen binding fragment thereof comprises heavy chain FR regions contained in SEQ ID NO: 149 and light chain FR regions contained in SEQ ID NO: 133. In some embodiments, the antibody or antigen binding fragment thereof comprises heavy chain FR regions contained in SEQ ID NO: 149 and light chain FR regions contained in SEQ ID NO: 134. In some embodiments, the antibody or antigen binding fragment thereof comprises heavy chain FR regions contained in SEQ ID NO: 149 and light chain FR regions contained in SEQ ID NO: 135. In some embodiments, the antibody or antigen binding fragment thereof comprises heavy chain FR regions contained in SEQ ID NO: 149 and light chain FR regions contained in SEQ ID NO: 136. In some embodiments, the antibody or antigen binding fragment thereof comprises heavy chain FR regions contained in SEQ ID NO: 149 and light chain FR regions contained in SEQ ID NO: 137. In some embodiments, the antibody or antigen binding fragment thereof comprises heavy chain FR regions contained in SEQ ID NO: 149 and light chain FR regions contained in SEQ ID NO: 138.

In some embodiments, the antibody or antigen binding fragment thereof comprises heavy chain FR regions contained in SEQ ID NO: 150 and light chain FR regions contained in SEQ ID NO: 114. In some embodiments, the antibody or antigen binding fragment thereof comprises heavy chain FR regions contained in SEQ ID NO: 150 and light chain FR regions contained in SEQ ID NO: 116. In some embodiments, the antibody or antigen binding fragment thereof comprises heavy chain FR regions contained in SEQ ID NO: 150 and light chain FR regions contained in SEQ ID NO: 117. In some embodiments, the antibody or antigen binding fragment thereof comprises heavy chain FR regions contained in SEQ ID NO: 150 and light chain FR regions contained in SEQ ID NO: 118. In some embodiments, the antibody or antigen binding fragment thereof comprises heavy chain FR regions contained in SEQ ID NO: 150 and light chain FR regions contained in SEQ ID NO: 119. In some embodiments, the antibody or antigen binding fragment thereof comprises heavy chain FR regions contained in SEQ ID NO: 150 and light chain FR regions contained in SEQ ID NO: 120. In some embodiments, the antibody or antigen binding fragment thereof comprises heavy chain FR regions contained in SEQ ID NO: 150 and light chain FR regions contained in SEQ ID NO: 121. In some embodiments, the antibody or antigen binding fragment thereof comprises heavy chain FR regions contained in SEQ ID NO: 150 and light chain FR regions contained in SEQ ID NO: 122. In some embodiments, the antibody or antigen binding fragment thereof comprises heavy chain FR regions contained in SEQ ID NO: 150 and light chain FR regions contained in SEQ ID NO: 123. In some embodiments, the antibody or antigen binding fragment thereof comprises heavy chain FR regions contained in SEQ ID NO: 150 and light chain FR regions contained in SEQ ID NO: 124. In some embodiments, the antibody or antigen binding fragment thereof comprises heavy chain FR regions contained in SEQ ID NO: 150 and light chain FR regions contained in SEQ ID NO: 125. In some embodiments, the antibody or antigen binding fragment thereof comprises heavy chain FR regions contained in SEQ ID NO: 150 and light chain FR regions contained in SEQ ID NO: 126. In some embodiments, the antibody or antigen binding fragment thereof comprises heavy chain FR regions contained in SEQ ID NO: 150 and light chain FR regions contained in SEQ ID NO: 127. In some embodiments, the antibody or antigen binding fragment thereof comprises heavy chain FR regions contained in SEQ ID NO: 150 and light chain FR regions contained in SEQ ID NO: 128. In some embodiments, the antibody or antigen binding fragment thereof comprises heavy chain FR regions contained in SEQ ID NO: 150 and light chain FR regions contained in SEQ ID NO: 129. In some embodiments, the antibody or antigen binding fragment thereof comprises heavy chain FR regions contained in SEQ ID NO: 150 and light chain FR regions contained in SEQ ID NO: 130. In some embodiments, the antibody or antigen binding fragment thereof comprises heavy chain FR regions contained in SEQ ID NO: 150 and light chain FR regions contained in SEQ ID NO: 131. In some embodiments, the antibody or antigen binding fragment thereof comprises heavy chain FR regions contained in SEQ ID NO: 150 and light chain FR regions contained in SEQ ID NO: 132. In some embodiments, the antibody or antigen binding fragment thereof comprises heavy chain FR regions contained in SEQ ID NO: 150 and light chain FR regions contained in SEQ ID NO: 133. In some embodiments, the antibody or antigen binding fragment thereof comprises heavy chain FR regions contained in SEQ ID NO: 150 and light chain FR regions contained in SEQ ID NO: 134. In some embodiments, the antibody or antigen binding fragment thereof comprises heavy chain FR regions contained in SEQ ID NO: 150 and light chain FR regions contained in SEQ ID NO: 135. In some embodiments, the antibody or antigen binding fragment thereof comprises heavy chain FR regions contained in SEQ ID NO: 150 and light chain FR regions contained in SEQ ID NO: 136. In some embodiments, the antibody or antigen binding fragment thereof comprises heavy chain FR regions contained in SEQ ID NO: 150 and light chain FR regions contained in SEQ ID NO: 137. In some embodiments, the antibody or antigen binding fragment thereof comprises heavy chain FR regions contained in SEQ ID NO: 150 and light chain FR regions contained in SEQ ID NO: 138.

In some embodiments, the antibody or antigen binding fragment thereof comprises heavy chain FR regions contained in SEQ ID NO: 151 and light chain FR regions contained in SEQ ID NO: 114. In some embodiments, the antibody or antigen binding fragment thereof comprises heavy chain FR regions contained in SEQ ID NO: 151 and light chain FR regions contained in SEQ ID NO: 116. In some embodiments, the antibody or antigen binding fragment thereof comprises heavy chain FR regions contained in SEQ ID NO: 151 and light chain FR regions contained in SEQ ID NO: 117. In some embodiments, the antibody or antigen binding fragment thereof comprises heavy chain FR regions contained in SEQ ID NO: 151 and light chain FR regions contained in SEQ ID NO: 118. In some embodiments, the antibody or antigen binding fragment thereof comprises heavy chain FR regions contained in SEQ ID NO: 151 and light chain FR regions contained in SEQ ID NO: 119. In some embodiments, the antibody or antigen binding fragment thereof comprises heavy chain FR regions contained in SEQ ID NO: 151 and light chain FR regions contained in SEQ ID NO: 120. In some embodiments, the antibody or antigen binding fragment thereof comprises heavy chain FR regions contained in SEQ ID NO: 151 and light chain FR regions contained in SEQ ID NO: 121. In some embodiments, the antibody or antigen binding fragment thereof comprises heavy chain FR regions contained in SEQ ID NO: 151 and light chain FR regions contained in SEQ ID NO: 122. In some embodiments, the antibody or antigen binding fragment thereof comprises heavy chain FR regions contained in SEQ ID NO: 151 and light chain FR regions contained in SEQ ID NO: 123. In some embodiments, the antibody or antigen binding fragment thereof comprises heavy chain FR regions contained in SEQ ID NO: 151 and light chain FR regions contained in SEQ ID NO: 124. In some embodiments, the antibody or antigen binding fragment thereof comprises heavy chain FR regions contained in SEQ ID NO: 151 and light chain FR regions contained in SEQ ID NO: 125. In some embodiments, the antibody or antigen binding fragment thereof comprises heavy chain FR regions contained in SEQ ID NO: 151 and light chain FR regions contained in SEQ ID NO: 126. In some embodiments, the antibody or antigen binding fragment thereof comprises heavy chain FR regions contained in SEQ ID NO: 151 and light chain FR regions contained in SEQ ID NO: 127. In some embodiments, the antibody or antigen binding fragment thereof comprises heavy chain FR regions contained in SEQ ID NO: 151 and light chain FR regions contained in SEQ ID NO: 128. In some embodiments, the antibody or antigen binding fragment thereof comprises heavy chain FR regions contained in SEQ ID NO: 151 and light chain FR regions contained in SEQ ID NO: 129. In some embodiments, the antibody or antigen binding fragment thereof comprises heavy chain FR regions contained in SEQ ID NO: 151 and light chain FR regions contained in SEQ ID NO: 130. In some embodiments, the antibody or antigen binding fragment thereof comprises heavy chain FR regions contained in SEQ ID NO: 151 and light chain FR regions contained in SEQ ID NO: 131. In some embodiments, the antibody or antigen binding fragment thereof comprises heavy chain FR regions contained in SEQ ID NO: 151 and light chain FR regions contained in SEQ ID NO: 132. In some embodiments, the antibody or antigen binding fragment thereof comprises heavy chain FR regions contained in SEQ ID NO: 151 and light chain FR regions contained in SEQ ID NO: 133. In some embodiments, the antibody or antigen binding fragment thereof comprises heavy chain FR regions contained in SEQ ID NO: 151 and light chain FR regions contained in SEQ ID NO: 134. In some embodiments, the antibody or antigen binding fragment thereof comprises heavy chain FR regions contained in SEQ ID NO: 151 and light chain FR regions contained in SEQ ID NO: 135. In some embodiments, the antibody or antigen binding fragment thereof comprises heavy chain FR regions contained in SEQ ID NO: 151 and light chain FR regions contained in SEQ ID NO: 136. In some embodiments, the antibody or antigen binding fragment thereof comprises heavy chain FR regions contained in SEQ ID NO: 151 and light chain FR regions contained in SEQ ID NO: 137. In some embodiments, the antibody or antigen binding fragment thereof comprises heavy chain FR regions contained in SEQ ID NO: 151 and light chain FR regions contained in SEQ ID NO: 138.

In some embodiments, the antibody or antigen binding fragment thereof comprises heavy chain FR regions contained in SEQ ID NO: 152 and light chain FR regions contained in SEQ ID NO: 114. In some embodiments, the antibody or antigen binding fragment thereof comprises heavy chain FR regions contained in SEQ ID NO: 152 and light chain FR regions contained in SEQ ID NO: 116. In some embodiments, the antibody or antigen binding fragment thereof comprises heavy chain FR regions contained in SEQ ID NO: 152 and light chain FR regions contained in SEQ ID NO: 117. In some embodiments, the antibody or antigen binding fragment thereof comprises heavy chain FR regions contained in SEQ ID NO: 152 and light chain FR regions contained in SEQ ID NO: 118. In some embodiments, the antibody or antigen binding fragment thereof comprises heavy chain FR regions contained in SEQ ID NO: 152 and light chain FR regions contained in SEQ ID NO: 119. In some embodiments, the antibody or antigen binding fragment thereof comprises heavy chain FR regions contained in SEQ ID NO: 152 and light chain FR regions contained in SEQ ID NO: 120. In some embodiments, the antibody or antigen binding fragment thereof comprises heavy chain FR regions contained in SEQ ID NO: 152 and light chain FR regions contained in SEQ ID NO: 121. In some embodiments, the antibody or antigen binding fragment thereof comprises heavy chain FR regions contained in SEQ ID NO: 152 and light chain FR regions contained in SEQ ID NO: 122. In some embodiments, the antibody or antigen binding fragment thereof comprises heavy chain FR regions contained in SEQ ID NO: 152 and light chain FR regions contained in SEQ ID NO: 123. In some embodiments, the antibody or antigen binding fragment thereof comprises heavy chain FR regions contained in SEQ ID NO: 152 and light chain FR regions contained in SEQ ID NO: 124. In some embodiments, the antibody or antigen binding fragment thereof comprises heavy chain FR regions contained in SEQ ID NO: 152 and light chain FR regions contained in SEQ ID NO: 125. In some embodiments, the antibody or antigen binding fragment thereof comprises heavy chain FR regions contained in SEQ ID NO: 152 and light chain FR regions contained in SEQ ID NO: 126. In some embodiments, the antibody or antigen binding fragment thereof comprises heavy chain FR regions contained in SEQ ID NO: 152 and light chain FR regions contained in SEQ ID NO: 127. In some embodiments, the antibody or antigen binding fragment thereof comprises heavy chain FR regions contained in SEQ ID NO: 152 and light chain FR regions contained in SEQ ID NO: 128. In some embodiments, the antibody or antigen binding fragment thereof comprises heavy chain FR regions contained in SEQ ID NO: 152 and light chain FR regions contained in SEQ ID NO: 129. In some embodiments, the antibody or antigen binding fragment thereof comprises heavy chain FR regions contained in SEQ ID NO: 152 and light chain FR regions contained in SEQ ID NO: 130. In some embodiments, the antibody or antigen binding fragment thereof comprises heavy chain FR regions contained in SEQ ID NO: 152 and light chain FR regions contained in SEQ ID NO: 131. In some embodiments, the antibody or antigen binding fragment thereof comprises heavy chain FR regions contained in SEQ ID NO: 152 and light chain FR regions contained in SEQ ID NO: 132. In some embodiments, the antibody or antigen binding fragment thereof comprises heavy chain FR regions contained in SEQ ID NO: 152 and light chain FR regions contained in SEQ ID NO: 133. In some embodiments, the antibody or antigen binding fragment thereof comprises heavy chain FR regions contained in SEQ ID NO: 152 and light chain FR regions contained in SEQ ID NO: 134. In some embodiments, the antibody or antigen binding fragment thereof comprises heavy chain FR regions contained in SEQ ID NO: 152 and light chain FR regions contained in SEQ ID NO: 135. In some embodiments, the antibody or antigen binding fragment thereof comprises heavy chain FR regions contained in SEQ ID NO: 152 and light chain FR regions contained in SEQ ID NO: 136. In some embodiments, the antibody or antigen binding fragment thereof comprises heavy chain FR regions contained in SEQ ID NO: 152 and light chain FR regions contained in SEQ ID NO: 137. In some embodiments, the antibody or antigen binding fragment thereof comprises heavy chain FR regions contained in SEQ ID NO: 152 and light chain FR regions contained in SEQ ID NO: 138.

In some embodiments, the antibody or antigen binding fragment thereof comprises heavy chain FR regions contained in SEQ ID NO: 153 and light chain FR regions contained in SEQ ID NO: 114. In some embodiments, the antibody or antigen binding fragment thereof comprises heavy chain FR regions contained in SEQ ID NO: 153 and light chain FR regions contained in SEQ ID NO: 116. In some embodiments, the antibody or antigen binding fragment thereof comprises heavy chain FR regions contained in SEQ ID NO: 153 and light chain FR regions contained in SEQ ID NO: 117. In some embodiments, the antibody or antigen binding fragment thereof comprises heavy chain FR regions contained in SEQ ID NO: 153 and light chain FR regions contained in SEQ ID NO: 118. In some embodiments, the antibody or antigen binding fragment thereof comprises heavy chain FR regions contained in SEQ ID NO: 153 and light chain FR regions contained in SEQ ID NO: 119. In some embodiments, the antibody or antigen binding fragment thereof comprises heavy chain FR regions contained in SEQ ID NO: 153 and light chain FR regions contained in SEQ ID NO: 120. In some embodiments, the antibody or antigen binding fragment thereof comprises heavy chain FR regions contained in SEQ ID NO: 153 and light chain FR regions contained in SEQ ID NO: 121. In some embodiments, the antibody or antigen binding fragment thereof comprises heavy chain FR regions contained in SEQ ID NO: 153 and light chain FR regions contained in SEQ ID NO: 122. In some embodiments, the antibody or antigen binding fragment thereof comprises heavy chain FR regions contained in SEQ ID NO: 153 and light chain FR regions contained in SEQ ID NO: 123. In some embodiments, the antibody or antigen binding fragment thereof comprises heavy chain FR regions contained in SEQ ID NO: 153 and light chain FR regions contained in SEQ ID NO: 124. In some embodiments, the antibody or antigen binding fragment thereof comprises heavy chain FR regions contained in SEQ ID NO: 153 and light chain FR regions contained in SEQ ID NO: 125. In some embodiments, the antibody or antigen binding fragment thereof comprises heavy chain FR regions contained in SEQ ID NO: 153 and light chain FR regions contained in SEQ ID NO: 126. In some embodiments, the antibody or antigen binding fragment thereof comprises heavy chain FR regions contained in SEQ ID NO: 153 and light chain FR regions contained in SEQ ID NO: 127. In some embodiments, the antibody or antigen binding fragment thereof comprises heavy chain FR regions contained in SEQ ID NO: 153 and light chain FR regions contained in SEQ ID NO: 128. In some embodiments, the antibody or antigen binding fragment thereof comprises heavy chain FR regions contained in SEQ ID NO: 153 and light chain FR regions contained in SEQ ID NO: 129. In some embodiments, the antibody or antigen binding fragment thereof comprises heavy chain FR regions contained in SEQ ID NO: 153 and light chain FR regions contained in SEQ ID NO: 130. In some embodiments, the antibody or antigen binding fragment thereof comprises heavy chain FR regions contained in SEQ ID NO: 153 and light chain FR regions contained in SEQ ID NO: 131. In some embodiments, the antibody or antigen binding fragment thereof comprises heavy chain FR regions contained in SEQ ID NO: 153 and light chain FR regions contained in SEQ ID NO: 132. In some embodiments, the antibody or antigen binding fragment thereof comprises heavy chain FR regions contained in SEQ ID NO: 153 and light chain FR regions contained in SEQ ID NO: 133. In some embodiments, the antibody or antigen binding fragment thereof comprises heavy chain FR regions contained in SEQ ID NO: 153 and light chain FR regions contained in SEQ ID NO: 134. In some embodiments, the antibody or antigen binding fragment thereof comprises heavy chain FR regions contained in SEQ ID NO: 153 and light chain FR regions contained in SEQ ID NO: 135. In some embodiments, the antibody or antigen binding fragment thereof comprises heavy chain FR regions contained in SEQ ID NO: 153 and light chain FR regions contained in SEQ ID NO: 136. In some embodiments, the antibody or antigen binding fragment thereof comprises heavy chain FR regions contained in SEQ ID NO: 153 and light chain FR regions contained in SEQ ID NO: 137. In some embodiments, the antibody or antigen binding fragment thereof comprises heavy chain FR regions contained in SEQ ID NO: 153 and light chain FR regions contained in SEQ ID NO: 138.

In some embodiments, the antibody or antigen binding fragment thereof comprises heavy chain FR regions contained in SEQ ID NO: 154 and light chain FR regions contained in SEQ ID NO: 114. In some embodiments, the antibody or antigen binding fragment thereof comprises heavy chain FR regions contained in SEQ ID NO: 154 and light chain FR regions contained in SEQ ID NO: 116. In some embodiments, the antibody or antigen binding fragment thereof comprises heavy chain FR regions contained in SEQ ID NO: 154 and light chain FR regions contained in SEQ ID NO: 117. In some embodiments, the antibody or antigen binding fragment thereof comprises heavy chain FR regions contained in SEQ ID NO: 154 and light chain FR regions contained in SEQ ID NO: 118. In some embodiments, the antibody or antigen binding fragment thereof comprises heavy chain FR regions contained in SEQ ID NO: 154 and light chain FR regions contained in SEQ ID NO: 119. In some embodiments, the antibody or antigen binding fragment thereof comprises heavy chain FR regions contained in SEQ ID NO: 154 and light chain FR regions contained in SEQ ID NO: 120. In some embodiments, the antibody or antigen binding fragment thereof comprises heavy chain FR regions contained in SEQ ID NO: 154 and light chain FR regions contained in SEQ ID NO: 121. In some embodiments, the antibody or antigen binding fragment thereof comprises heavy chain FR regions contained in SEQ ID NO: 154 and light chain FR regions contained in SEQ ID NO: 122. In some embodiments, the antibody or antigen binding fragment thereof comprises heavy chain FR regions contained in SEQ ID NO: 154 and light chain FR regions contained in SEQ ID NO: 123. In some embodiments, the antibody or antigen binding fragment thereof comprises heavy chain FR regions contained in SEQ ID NO: 154 and light chain FR regions contained in SEQ ID NO: 124. In some embodiments, the antibody or antigen binding fragment thereof comprises heavy chain FR regions contained in SEQ ID NO: 154 and light chain FR regions contained in SEQ ID NO: 125. In some embodiments, the antibody or antigen binding fragment thereof comprises heavy chain FR regions contained in SEQ ID NO: 154 and light chain FR regions contained in SEQ ID NO: 126. In some embodiments, the antibody or antigen binding fragment thereof comprises heavy chain FR regions contained in SEQ ID NO: 154 and light chain FR regions contained in SEQ ID NO: 127. In some embodiments, the antibody or antigen binding fragment thereof comprises heavy chain FR regions contained in SEQ ID NO: 154 and light chain FR regions contained in SEQ ID NO: 128. In some embodiments, the antibody or antigen binding fragment thereof comprises heavy chain FR regions contained in SEQ ID NO: 154 and light chain FR regions contained in SEQ ID NO: 129. In some embodiments, the antibody or antigen binding fragment thereof comprises heavy chain FR regions contained in SEQ ID NO: 154 and light chain FR regions contained in SEQ ID NO: 130. In some embodiments, the antibody or antigen binding fragment thereof comprises heavy chain FR regions contained in SEQ ID NO: 154 and light chain FR regions contained in SEQ ID NO: 131. In some embodiments, the antibody or antigen binding fragment thereof comprises heavy chain FR regions contained in SEQ ID NO: 154 and light chain FR regions contained in SEQ ID NO: 132. In some embodiments, the antibody or antigen binding fragment thereof comprises heavy chain FR regions contained in SEQ ID NO: 154 and light chain FR regions contained in SEQ ID NO: 133. In some embodiments, the antibody or antigen binding fragment thereof comprises heavy chain FR regions contained in SEQ ID NO: 154 and light chain FR regions contained in SEQ ID NO: 134. In some embodiments, the antibody or antigen binding fragment thereof comprises heavy chain FR regions contained in SEQ ID NO: 154 and light chain FR regions contained in SEQ ID NO: 135. In some embodiments, the antibody or antigen binding fragment thereof comprises heavy chain FR regions contained in SEQ ID NO: 154 and light chain FR regions contained in SEQ ID NO: 136. In some embodiments, the antibody or antigen binding fragment thereof comprises heavy chain FR regions contained in SEQ ID NO: 154 and light chain FR regions contained in SEQ ID NO: 137. In some embodiments, the antibody or antigen binding fragment thereof comprises heavy chain FR regions contained in SEQ ID NO: 154 and light chain FR regions contained in SEQ ID NO: 138.

In some embodiments, the antibody or antigen binding fragment thereof comprises heavy chain FR regions contained in SEQ ID NO: 155 and light chain FR regions contained in SEQ ID NO: 114. In some embodiments, the antibody or antigen binding fragment thereof comprises heavy chain FR regions contained in SEQ ID NO: 155 and light chain FR regions contained in SEQ ID NO: 116. In some embodiments, the antibody or antigen binding fragment thereof comprises heavy chain FR regions contained in SEQ ID NO: 155 and light chain FR regions contained in SEQ ID NO: 117. In some embodiments, the antibody or antigen binding fragment thereof comprises heavy chain FR regions contained in SEQ ID NO: 155 and light chain FR regions contained in SEQ ID NO: 118. In some embodiments, the antibody or antigen binding fragment thereof comprises heavy chain FR regions contained in SEQ ID NO: 155 and light chain FR regions contained in SEQ ID NO: 119. In some embodiments, the antibody or antigen binding fragment thereof comprises heavy chain FR regions contained in SEQ ID NO: 155 and light chain FR regions contained in SEQ ID NO: 120. In some embodiments, the antibody or antigen binding fragment thereof comprises heavy chain FR regions contained in SEQ ID NO: 155 and light chain FR regions contained in SEQ ID NO: 121. In some embodiments, the antibody or antigen binding fragment thereof comprises heavy chain FR regions contained in SEQ ID NO: 155 and light chain FR regions contained in SEQ ID NO: 122. In some embodiments, the antibody or antigen binding fragment thereof comprises heavy chain FR regions contained in SEQ ID NO: 155 and light chain FR regions contained in SEQ ID NO: 123. In some embodiments, the antibody or antigen binding fragment thereof comprises heavy chain FR regions contained in SEQ ID NO: 155 and light chain FR regions contained in SEQ ID NO: 124. In some embodiments, the antibody or antigen binding fragment thereof comprises heavy chain FR regions contained in SEQ ID NO: 155 and light chain FR regions contained in SEQ ID NO: 125. In some embodiments, the antibody or antigen binding fragment thereof comprises heavy chain FR regions contained in SEQ ID NO: 155 and light chain FR regions contained in SEQ ID NO: 126. In some embodiments, the antibody or antigen binding fragment thereof comprises heavy chain FR regions contained in SEQ ID NO: 155 and light chain FR regions contained in SEQ ID NO: 127. In some embodiments, the antibody or antigen binding fragment thereof comprises heavy chain FR regions contained in SEQ ID NO: 155 and light chain FR regions contained in SEQ ID NO: 128. In some embodiments, the antibody or antigen binding fragment thereof comprises heavy chain FR regions contained in SEQ ID NO: 155 and light chain FR regions contained in SEQ ID NO: 129. In some embodiments, the antibody or antigen binding fragment thereof comprises heavy chain FR regions contained in SEQ ID NO: 155 and light chain FR regions contained in SEQ ID NO: 130. In some embodiments, the antibody or antigen binding fragment thereof comprises heavy chain FR regions contained in SEQ ID NO: 155 and light chain FR regions contained in SEQ ID NO: 131. In some embodiments, the antibody or antigen binding fragment thereof comprises heavy chain FR regions contained in SEQ ID NO: 155 and light chain FR regions contained in SEQ ID NO: 132. In some embodiments, the antibody or antigen binding fragment thereof comprises heavy chain FR regions contained in SEQ ID NO: 155 and light chain FR regions contained in SEQ ID NO: 133. In some embodiments, the antibody or antigen binding fragment thereof comprises heavy chain FR regions contained in SEQ ID NO: 155 and light chain FR regions contained in SEQ ID NO: 134. In some embodiments, the antibody or antigen binding fragment thereof comprises heavy chain FR regions contained in SEQ ID NO: 155 and light chain FR regions contained in SEQ ID NO: 135. In some embodiments, the antibody or antigen binding fragment thereof comprises heavy chain FR regions contained in SEQ ID NO: 155 and light chain FR regions contained in SEQ ID NO: 136. In some embodiments, the antibody or antigen binding fragment thereof comprises heavy chain FR regions contained in SEQ ID NO: 155 and light chain FR regions contained in SEQ ID NO: 137. In some embodiments, the antibody or antigen binding fragment thereof comprises heavy chain FR regions contained in SEQ ID NO: 155 and light chain FR regions contained in SEQ ID NO: 138.

In some embodiments, the antibody or antigen binding fragment thereof comprises heavy chain FR regions contained in SEQ ID NO: 156 and light chain FR regions contained in SEQ ID NO: 114. In some embodiments, the antibody or antigen binding fragment thereof comprises heavy chain FR regions contained in SEQ ID NO: 156 and light chain FR regions contained in SEQ ID NO: 116. In some embodiments, the antibody or antigen binding fragment thereof comprises heavy chain FR regions contained in SEQ ID NO: 156 and light chain FR regions contained in SEQ ID NO: 117. In some embodiments, the antibody or antigen binding fragment thereof comprises heavy chain FR regions contained in SEQ ID NO: 156 and light chain FR regions contained in SEQ ID NO: 118. In some embodiments, the antibody or antigen binding fragment thereof comprises heavy chain FR regions contained in SEQ ID NO: 156 and light chain FR regions contained in SEQ ID NO: 119. In some embodiments, the antibody or antigen binding fragment thereof comprises heavy chain FR regions contained in SEQ ID NO: 156 and light chain FR regions contained in SEQ ID NO: 120. In some embodiments, the antibody or antigen binding fragment thereof comprises heavy chain FR regions contained in SEQ ID NO: 156 and light chain FR regions contained in SEQ ID NO: 121. In some embodiments, the antibody or antigen binding fragment thereof comprises heavy chain FR regions contained in SEQ ID NO: 156 and light chain FR regions contained in SEQ ID NO: 122. In some embodiments, the antibody or antigen binding fragment thereof comprises heavy chain FR regions contained in SEQ ID NO: 156 and light chain FR regions contained in SEQ ID NO: 123. In some embodiments, the antibody or antigen binding fragment thereof comprises heavy chain FR regions contained in SEQ ID NO: 156 and light chain FR regions contained in SEQ ID NO: 124. In some embodiments, the antibody or antigen binding fragment thereof comprises heavy chain FR regions contained in SEQ ID NO: 156 and light chain FR regions contained in SEQ ID NO: 125. In some embodiments, the antibody or antigen binding fragment thereof comprises heavy chain FR regions contained in SEQ ID NO: 156 and light chain FR regions contained in SEQ ID NO: 126. In some embodiments, the antibody or antigen binding fragment thereof comprises heavy chain FR regions contained in SEQ ID NO: 156 and light chain FR regions contained in SEQ ID NO: 127. In some embodiments, the antibody or antigen binding fragment thereof comprises heavy chain FR regions contained in SEQ ID NO: 156 and light chain FR regions contained in SEQ ID NO: 128. In some embodiments, the antibody or antigen binding fragment thereof comprises heavy chain FR regions contained in SEQ ID NO: 156 and light chain FR regions contained in SEQ ID NO: 129. In some embodiments, the antibody or antigen binding fragment thereof comprises heavy chain FR regions contained in SEQ ID NO: 156 and light chain FR regions contained in SEQ ID NO: 130. In some embodiments, the antibody or antigen binding fragment thereof comprises heavy chain FR regions contained in SEQ ID NO: 156 and light chain FR regions contained in SEQ ID NO: 131. In some embodiments, the antibody or antigen binding fragment thereof comprises heavy chain FR regions contained in SEQ ID NO: 156 and light chain FR regions contained in SEQ ID NO: 132. In some embodiments, the antibody or antigen binding fragment thereof comprises heavy chain FR regions contained in SEQ ID NO: 156 and light chain FR regions contained in SEQ ID NO: 133. In some embodiments, the antibody or antigen binding fragment thereof comprises heavy chain FR regions contained in SEQ ID NO: 156 and light chain FR regions contained in SEQ ID NO: 134. In some embodiments, the antibody or antigen binding fragment thereof comprises heavy chain FR regions contained in SEQ ID NO: 156 and light chain FR regions contained in SEQ ID NO: 135. In some embodiments, the antibody or antigen binding fragment thereof comprises heavy chain FR regions contained in SEQ ID NO: 156 and light chain FR regions contained in SEQ ID NO: 136. In some embodiments, the antibody or antigen binding fragment thereof comprises heavy chain FR regions contained in SEQ ID NO: 156 and light chain FR regions contained in SEQ ID NO: 137. In some embodiments, the antibody or antigen binding fragment thereof comprises heavy chain FR regions contained in SEQ ID NO: 156 and light chain FR regions contained in SEQ ID NO: 138.

In some embodiments, the antibody or antigen binding fragment thereof comprises heavy chain FR regions contained in SEQ ID NO: 157 and light chain FR regions contained in SEQ ID NO: 114. In some embodiments, the antibody or antigen binding fragment thereof comprises heavy chain FR regions contained in SEQ ID NO: 157 and light chain FR regions contained in SEQ ID NO: 116. In some embodiments, the antibody or antigen binding fragment thereof comprises heavy chain FR regions contained in SEQ ID NO: 157 and light chain FR regions contained in SEQ ID NO: 117. In some embodiments, the antibody or antigen binding fragment thereof comprises heavy chain FR regions contained in SEQ ID NO: 157 and light chain FR regions contained in SEQ ID NO: 118. In some embodiments, the antibody or antigen binding fragment thereof comprises heavy chain FR regions contained in SEQ ID NO: 157 and light chain FR regions contained in SEQ ID NO: 119. In some embodiments, the antibody or antigen binding fragment thereof comprises heavy chain FR regions contained in SEQ ID NO: 157 and light chain FR regions contained in SEQ ID NO: 120. In some embodiments, the antibody or antigen binding fragment thereof comprises heavy chain FR regions contained in SEQ ID NO: 157 and light chain FR regions contained in SEQ ID NO: 121. In some embodiments, the antibody or antigen binding fragment thereof comprises heavy chain FR regions contained in SEQ ID NO: 157 and light chain FR regions contained in SEQ ID NO: 122. In some embodiments, the antibody or antigen binding fragment thereof comprises heavy chain FR regions contained in SEQ ID NO: 157 and light chain FR regions contained in SEQ ID NO: 123. In some embodiments, the antibody or antigen binding fragment thereof comprises heavy chain FR regions contained in SEQ ID NO: 157 and light chain FR regions contained in SEQ ID NO: 124. In some embodiments, the antibody or antigen binding fragment thereof comprises heavy chain FR regions contained in SEQ ID NO: 157 and light chain FR regions contained in SEQ ID NO: 125. In some embodiments, the antibody or antigen binding fragment thereof comprises heavy chain FR regions contained in SEQ ID NO: 157 and light chain FR regions contained in SEQ ID NO: 126. In some embodiments, the antibody or antigen binding fragment thereof comprises heavy chain FR regions contained in SEQ ID NO: 157 and light chain FR regions contained in SEQ ID NO: 127. In some embodiments, the antibody or antigen binding fragment thereof comprises heavy chain FR regions contained in SEQ ID NO: 157 and light chain FR regions contained in SEQ ID NO: 128. In some embodiments, the antibody or antigen binding fragment thereof comprises heavy chain FR regions contained in SEQ ID NO: 157 and light chain FR regions contained in SEQ ID NO: 129. In some embodiments, the antibody or antigen binding fragment thereof comprises heavy chain FR regions contained in SEQ ID NO: 157 and light chain FR regions contained in SEQ ID NO: 130. In some embodiments, the antibody or antigen binding fragment thereof comprises heavy chain FR regions contained in SEQ ID NO: 157 and light chain FR regions contained in SEQ ID NO: 131. In some embodiments, the antibody or antigen binding fragment thereof comprises heavy chain FR regions contained in SEQ ID NO: 157 and light chain FR regions contained in SEQ ID NO: 132. In some embodiments, the antibody or antigen binding fragment thereof comprises heavy chain FR regions contained in SEQ ID NO: 157 and light chain FR regions contained in SEQ ID NO: 133. In some embodiments, the antibody or antigen binding fragment thereof comprises heavy chain FR regions contained in SEQ ID NO: 157 and light chain FR regions contained in SEQ ID NO: 134. In some embodiments, the antibody or antigen binding fragment thereof comprises heavy chain FR regions contained in SEQ ID NO: 157 and light chain FR regions contained in SEQ ID NO: 135. In some embodiments, the antibody or antigen binding fragment thereof comprises heavy chain FR regions contained in SEQ ID NO: 157 and light chain FR regions contained in SEQ ID NO: 136. In some embodiments, the antibody or antigen binding fragment thereof comprises heavy chain FR regions contained in SEQ ID NO: 157 and light chain FR regions contained in SEQ ID NO: 137. In some embodiments, the antibody or antigen binding fragment thereof comprises heavy chain FR regions contained in SEQ ID NO: 157 and light chain FR regions contained in SEQ ID NO: 138.

In some embodiments, the antibody or antigen binding fragment thereof comprises heavy chain FR regions contained in SEQ ID NO: 158 and light chain FR regions contained in SEQ ID NO: 114. In some embodiments, the antibody or antigen binding fragment thereof comprises heavy chain FR regions contained in SEQ ID NO: 158 and light chain FR regions contained in SEQ ID NO: 116. In some embodiments, the antibody or antigen binding fragment thereof comprises heavy chain FR regions contained in SEQ ID NO: 158 and light chain FR regions contained in SEQ ID NO: 117. In some embodiments, the antibody or antigen binding fragment thereof comprises heavy chain FR regions contained in SEQ ID NO: 158 and light chain FR regions contained in SEQ ID NO: 118. In some embodiments, the antibody or antigen binding fragment thereof comprises heavy chain FR regions contained in SEQ ID NO: 158 and light chain FR regions contained in SEQ ID NO: 119. In some embodiments, the antibody or antigen binding fragment thereof comprises heavy chain FR regions contained in SEQ ID NO: 158 and light chain FR regions contained in SEQ ID NO: 120. In some embodiments, the antibody or antigen binding fragment thereof comprises heavy chain FR regions contained in SEQ ID NO: 158 and light chain FR regions contained in SEQ ID NO: 121. In some embodiments, the antibody or antigen binding fragment thereof comprises heavy chain FR regions contained in SEQ ID NO: 158 and light chain FR regions contained in SEQ ID NO: 122. In some embodiments, the antibody or antigen binding fragment thereof comprises heavy chain FR regions contained in SEQ ID NO: 158 and light chain FR regions contained in SEQ ID NO: 123. In some embodiments, the antibody or antigen binding fragment thereof comprises heavy chain FR regions contained in SEQ ID NO: 158 and light chain FR regions contained in SEQ ID NO: 124. In some embodiments, the antibody or antigen binding fragment thereof comprises heavy chain FR regions contained in SEQ ID NO: 158 and light chain FR regions contained in SEQ ID NO: 125. In some embodiments, the antibody or antigen binding fragment thereof comprises heavy chain FR regions contained in SEQ ID NO: 158 and light chain FR regions contained in SEQ ID NO: 126. In some embodiments, the antibody or antigen binding fragment thereof comprises heavy chain FR regions contained in SEQ ID NO: 158 and light chain FR regions contained in SEQ ID NO: 127. In some embodiments, the antibody or antigen binding fragment thereof comprises heavy chain FR regions contained in SEQ ID NO: 158 and light chain FR regions contained in SEQ ID NO: 128. In some embodiments, the antibody or antigen binding fragment thereof comprises heavy chain FR regions contained in SEQ ID NO: 158 and light chain FR regions contained in SEQ ID NO: 129. In some embodiments, the antibody or antigen binding fragment thereof comprises heavy chain FR regions contained in SEQ ID NO: 158 and light chain FR regions contained in SEQ ID NO: 130. In some embodiments, the antibody or antigen binding fragment thereof comprises heavy chain FR regions contained in SEQ ID NO: 158 and light chain FR regions contained in SEQ ID NO: 131. In some embodiments, the antibody or antigen binding fragment thereof comprises heavy chain FR regions contained in SEQ ID NO: 158 and light chain FR regions contained in SEQ ID NO: 132. In some embodiments, the antibody or antigen binding fragment thereof comprises heavy chain FR regions contained in SEQ ID NO: 158 and light chain FR regions contained in SEQ ID NO: 133. In some embodiments, the antibody or antigen binding fragment thereof comprises heavy chain FR regions contained in SEQ ID NO: 158 and light chain FR regions contained in SEQ ID NO: 134. In some embodiments, the antibody or antigen binding fragment thereof comprises heavy chain FR regions contained in SEQ ID NO: 158 and light chain FR regions contained in SEQ ID NO: 135. In some embodiments, the antibody or antigen binding fragment thereof comprises heavy chain FR regions contained in SEQ ID NO: 158 and light chain FR regions contained in SEQ ID NO: 136. In some embodiments, the antibody or antigen binding fragment thereof comprises heavy chain FR regions contained in SEQ ID NO: 158 and light chain FR regions contained in SEQ ID NO: 137. In some embodiments, the antibody or antigen binding fragment thereof comprises heavy chain FR regions contained in SEQ ID NO: 158 and light chain FR regions contained in SEQ ID NO: 138.

In some embodiments, the antibody or antigen binding fragment thereof comprises heavy chain FR regions contained in SEQ ID NO: 159 and light chain FR regions contained in SEQ ID NO: 114. In some embodiments, the antibody or antigen binding fragment thereof comprises heavy chain FR regions contained in SEQ ID NO: 159 and light chain FR regions contained in SEQ ID NO: 116. In some embodiments, the antibody or antigen binding fragment thereof comprises heavy chain FR regions contained in SEQ ID NO: 159 and light chain FR regions contained in SEQ ID NO: 117. In some embodiments, the antibody or antigen binding fragment thereof comprises heavy chain FR regions contained in SEQ ID NO: 159 and light chain FR regions contained in SEQ ID NO: 118. In some embodiments, the antibody or antigen binding fragment thereof comprises heavy chain FR regions contained in SEQ ID NO: 159 and light chain FR regions contained in SEQ ID NO: 119. In some embodiments, the antibody or antigen binding fragment thereof comprises heavy chain FR regions contained in SEQ ID NO: 159 and light chain FR regions contained in SEQ ID NO: 120. In some embodiments, the antibody or antigen binding fragment thereof comprises heavy chain FR regions contained in SEQ ID NO: 159 and light chain FR regions contained in SEQ ID NO: 121. In some embodiments, the antibody or antigen binding fragment thereof comprises heavy chain FR regions contained in SEQ ID NO: 159 and light chain FR regions contained in SEQ ID NO: 122. In some embodiments, the antibody or antigen binding fragment thereof comprises heavy chain FR regions contained in SEQ ID NO: 159 and light chain FR regions contained in SEQ ID NO: 123. In some embodiments, the antibody or antigen binding fragment thereof comprises heavy chain FR regions contained in SEQ ID NO: 159 and light chain FR regions contained in SEQ ID NO: 124. In some embodiments, the antibody or antigen binding fragment thereof comprises heavy chain FR regions contained in SEQ ID NO: 159 and light chain FR regions contained in SEQ ID NO: 125. In some embodiments, the antibody or antigen binding fragment thereof comprises heavy chain FR regions contained in SEQ ID NO: 159 and light chain FR regions contained in SEQ ID NO: 126. In some embodiments, the antibody or antigen binding fragment thereof comprises heavy chain FR regions contained in SEQ ID NO: 159 and light chain FR regions contained in SEQ ID NO: 127. In some embodiments, the antibody or antigen binding fragment thereof comprises heavy chain FR regions contained in SEQ ID NO: 159 and light chain FR regions contained in SEQ ID NO: 128. In some embodiments, the antibody or antigen binding fragment thereof comprises heavy chain FR regions contained in SEQ ID NO: 159 and light chain FR regions contained in SEQ ID NO: 129. In some embodiments, the antibody or antigen binding fragment thereof comprises heavy chain FR regions contained in SEQ ID NO: 159 and light chain FR regions contained in SEQ ID NO: 130. In some embodiments, the antibody or antigen binding fragment thereof comprises heavy chain FR regions contained in SEQ ID NO: 159 and light chain FR regions contained in SEQ ID NO: 131. In some embodiments, the antibody or antigen binding fragment thereof comprises heavy chain FR regions contained in SEQ ID NO: 159 and light chain FR regions contained in SEQ ID NO: 132. In some embodiments, the antibody or antigen binding fragment thereof comprises heavy chain FR regions contained in SEQ ID NO: 159 and light chain FR regions contained in SEQ ID NO: 133. In some embodiments, the antibody or antigen binding fragment thereof comprises heavy chain FR regions contained in SEQ ID NO: 159 and light chain FR regions contained in SEQ ID NO: 134. In some embodiments, the antibody or antigen binding fragment thereof comprises heavy chain FR regions contained in SEQ ID NO: 159 and light chain FR regions contained in SEQ ID NO: 135. In some embodiments, the antibody or antigen binding fragment thereof comprises heavy chain FR regions contained in SEQ ID NO: 159 and light chain FR regions contained in SEQ ID NO: 136. In some embodiments, the antibody or antigen binding fragment thereof comprises heavy chain FR regions contained in SEQ ID NO: 159 and light chain FR regions contained in SEQ ID NO: 137. In some embodiments, the antibody or antigen binding fragment thereof comprises heavy chain FR regions contained in SEQ ID NO: 159 and light chain FR regions contained in SEQ ID NO: 138.

In some embodiments, the antibody or antigen binding fragment thereof comprises heavy chain FR regions contained in SEQ ID NO: 160 and light chain FR regions contained in SEQ ID NO: 114. In some embodiments, the antibody or antigen binding fragment thereof comprises heavy chain FR regions contained in SEQ ID NO: 160 and light chain FR regions contained in SEQ ID NO: 116. In some embodiments, the antibody or antigen binding fragment thereof comprises heavy chain FR regions contained in SEQ ID NO: 160 and light chain FR regions contained in SEQ ID NO: 117. In some embodiments, the antibody or antigen binding fragment thereof comprises heavy chain FR regions contained in SEQ ID NO: 160 and light chain FR regions contained in SEQ ID NO: 118. In some embodiments, the antibody or antigen binding fragment thereof comprises heavy chain FR regions contained in SEQ ID NO: 160 and light chain FR regions contained in SEQ ID NO: 119. In some embodiments, the antibody or antigen binding fragment thereof comprises heavy chain FR regions contained in SEQ ID NO: 160 and light chain FR regions contained in SEQ ID NO: 120. In some embodiments, the antibody or antigen binding fragment thereof comprises heavy chain FR regions contained in SEQ ID NO: 160 and light chain FR regions contained in SEQ ID NO: 121. In some embodiments, the antibody or antigen binding fragment thereof comprises heavy chain FR regions contained in SEQ ID NO: 160 and light chain FR regions contained in SEQ ID NO: 122. In some embodiments, the antibody or antigen binding fragment thereof comprises heavy chain FR regions contained in SEQ ID NO: 160 and light chain FR regions contained in SEQ ID NO: 123. In some embodiments, the antibody or antigen binding fragment thereof comprises heavy chain FR regions contained in SEQ ID NO: 160 and light chain FR regions contained in SEQ ID NO: 124. In some embodiments, the antibody or antigen binding fragment thereof comprises heavy chain FR regions contained in SEQ ID NO: 160 and light chain FR regions contained in SEQ ID NO: 125. In some embodiments, the antibody or antigen binding fragment thereof comprises heavy chain FR regions contained in SEQ ID NO: 160 and light chain FR regions contained in SEQ ID NO: 126. In some embodiments, the antibody or antigen binding fragment thereof comprises heavy chain FR regions contained in SEQ ID NO: 160 and light chain FR regions contained in SEQ ID NO: 127. In some embodiments, the antibody or antigen binding fragment thereof comprises heavy chain FR regions contained in SEQ ID NO: 160 and light chain FR regions contained in SEQ ID NO: 128. In some embodiments, the antibody or antigen binding fragment thereof comprises heavy chain FR regions contained in SEQ ID NO: 160 and light chain FR regions contained in SEQ ID NO: 129. In some embodiments, the antibody or antigen binding fragment thereof comprises heavy chain FR regions contained in SEQ ID NO: 160 and light chain FR regions contained in SEQ ID NO: 130. In some embodiments, the antibody or antigen binding fragment thereof comprises heavy chain FR regions contained in SEQ ID NO: 160 and light chain FR regions contained in SEQ ID NO: 131. In some embodiments, the antibody or antigen binding fragment thereof comprises heavy chain FR regions contained in SEQ ID NO: 160 and light chain FR regions contained in SEQ ID NO: 132. In some embodiments, the antibody or antigen binding fragment thereof comprises heavy chain FR regions contained in SEQ ID NO: 160 and light chain FR regions contained in SEQ ID NO: 133. In some embodiments, the antibody or antigen binding fragment thereof comprises heavy chain FR regions contained in SEQ ID NO: 160 and light chain FR regions contained in SEQ ID NO: 134. In some embodiments, the antibody or antigen binding fragment thereof comprises heavy chain FR regions contained in SEQ ID NO: 160 and light chain FR regions contained in SEQ ID NO: 135. In some embodiments, the antibody or antigen binding fragment thereof comprises heavy chain FR regions contained in SEQ ID NO: 160 and light chain FR regions contained in SEQ ID NO: 136. In some embodiments, the antibody or antigen binding fragment thereof comprises heavy chain FR regions contained in SEQ ID NO: 160 and light chain FR regions contained in SEQ ID NO: 137. In some embodiments, the antibody or antigen binding fragment thereof comprises heavy chain FR regions contained in SEQ ID NO: 160 and light chain FR regions contained in SEQ ID NO: 138.

In some embodiments, the antibody or antigen binding fragment thereof comprises heavy chain FR regions contained in SEQ ID NO: 161 and light chain FR regions contained in SEQ ID NO: 114. In some embodiments, the antibody or antigen binding fragment thereof comprises heavy chain FR regions contained in SEQ ID NO: 161 and light chain FR regions contained in SEQ ID NO: 116. In some embodiments, the antibody or antigen binding fragment thereof comprises heavy chain FR regions contained in SEQ ID NO: 161 and light chain FR regions contained in SEQ ID NO: 117. In some embodiments, the antibody or antigen binding fragment thereof comprises heavy chain FR regions contained in SEQ ID NO: 161 and light chain FR regions contained in SEQ ID NO: 118. In some embodiments, the antibody or antigen binding fragment thereof comprises heavy chain FR regions contained in SEQ ID NO: 161 and light chain FR regions contained in SEQ ID NO: 119. In some embodiments, the antibody or antigen binding fragment thereof comprises heavy chain FR regions contained in SEQ ID NO: 161 and light chain FR regions contained in SEQ ID NO: 120. In some embodiments, the antibody or antigen binding fragment thereof comprises heavy chain FR regions contained in SEQ ID NO: 161 and light chain FR regions contained in SEQ ID NO: 121. In some embodiments, the antibody or antigen binding fragment thereof comprises heavy chain FR regions contained in SEQ ID NO: 161 and light chain FR regions contained in SEQ ID NO: 122. In some embodiments, the antibody or antigen binding fragment thereof comprises heavy chain FR regions contained in SEQ ID NO: 161 and light chain FR regions contained in SEQ ID NO: 123. In some embodiments, the antibody or antigen binding fragment thereof comprises heavy chain FR regions contained in SEQ ID NO: 161 and light chain FR regions contained in SEQ ID NO: 124. In some embodiments, the antibody or antigen binding fragment thereof comprises heavy chain FR regions contained in SEQ ID NO: 161 and light chain FR regions contained in SEQ ID NO: 125. In some embodiments, the antibody or antigen binding fragment thereof comprises heavy chain FR regions contained in SEQ ID NO: 161 and light chain FR regions contained in SEQ ID NO: 126. In some embodiments, the antibody or antigen binding fragment thereof comprises heavy chain FR regions contained in SEQ ID NO: 161 and light chain FR regions contained in SEQ ID NO: 127. In some embodiments, the antibody or antigen binding fragment thereof comprises heavy chain FR regions contained in SEQ ID NO: 161 and light chain FR regions contained in SEQ ID NO: 128. In some embodiments, the antibody or antigen binding fragment thereof comprises heavy chain FR regions contained in SEQ ID NO: 161 and light chain FR regions contained in SEQ ID NO: 129. In some embodiments, the antibody or antigen binding fragment thereof comprises heavy chain FR regions contained in SEQ ID NO: 161 and light chain FR regions contained in SEQ ID NO: 130. In some embodiments, the antibody or antigen binding fragment thereof comprises heavy chain FR regions contained in SEQ ID NO: 161 and light chain FR regions contained in SEQ ID NO: 131. In some embodiments, the antibody or antigen binding fragment thereof comprises heavy chain FR regions contained in SEQ ID NO: 161 and light chain FR regions contained in SEQ ID NO: 132. In some embodiments, the antibody or antigen binding fragment thereof comprises heavy chain FR regions contained in SEQ ID NO: 161 and light chain FR regions contained in SEQ ID NO: 133. In some embodiments, the antibody or antigen binding fragment thereof comprises heavy chain FR regions contained in SEQ ID NO: 161 and light chain FR regions contained in SEQ ID NO: 134. In some embodiments, the antibody or antigen binding fragment thereof comprises heavy chain FR regions contained in SEQ ID NO: 161 and light chain FR regions contained in SEQ ID NO: 135. In some embodiments, the antibody or antigen binding fragment thereof comprises heavy chain FR regions contained in SEQ ID NO: 161 and light chain FR regions contained in SEQ ID NO: 136. In some embodiments, the antibody or antigen binding fragment thereof comprises heavy chain FR regions contained in SEQ ID NO: 161 and light chain FR regions contained in SEQ ID NO: 137. In some embodiments, the antibody or antigen binding fragment thereof comprises heavy chain FR regions contained in SEQ ID NO: 161 and light chain FR regions contained in SEQ ID NO: 138.

In some embodiments, the antibody or antigen binding fragment thereof comprises heavy chain FR regions contained in SEQ ID NO: 162 and light chain FR regions contained in SEQ ID NO: 114. In some embodiments, the antibody or antigen binding fragment thereof comprises heavy chain FR regions contained in SEQ ID NO: 162 and light chain FR regions contained in SEQ ID NO: 116. In some embodiments, the antibody or antigen binding fragment thereof comprises heavy chain FR regions contained in SEQ ID NO: 162 and light chain FR regions contained in SEQ ID NO: 117. In some embodiments, the antibody or antigen binding fragment thereof comprises heavy chain FR regions contained in SEQ ID NO: 162 and light chain FR regions contained in SEQ ID NO: 118. In some embodiments, the antibody or antigen binding fragment thereof comprises heavy chain FR regions contained in SEQ ID NO: 162 and light chain FR regions contained in SEQ ID NO: 119. In some embodiments, the antibody or antigen binding fragment thereof comprises heavy chain FR regions contained in SEQ ID NO: 162 and light chain FR regions contained in SEQ ID NO: 120. In some embodiments, the antibody or antigen binding fragment thereof comprises heavy chain FR regions contained in SEQ ID NO: 162 and light chain FR regions contained in SEQ ID NO: 121. In some embodiments, the antibody or antigen binding fragment thereof comprises heavy chain FR regions contained in SEQ ID NO: 162 and light chain FR regions contained in SEQ ID NO: 122. In some embodiments, the antibody or antigen binding fragment thereof comprises heavy chain FR regions contained in SEQ ID NO: 162 and light chain FR regions contained in SEQ ID NO: 123. In some embodiments, the antibody or antigen binding fragment thereof comprises heavy chain FR regions contained in SEQ ID NO: 162 and light chain FR regions contained in SEQ ID NO: 124. In some embodiments, the antibody or antigen binding fragment thereof comprises heavy chain FR regions contained in SEQ ID NO: 162 and light chain FR regions contained in SEQ ID NO: 125. In some embodiments, the antibody or antigen binding fragment thereof comprises heavy chain FR regions contained in SEQ ID NO: 162 and light chain FR regions contained in SEQ ID NO: 126. In some embodiments, the antibody or antigen binding fragment thereof comprises heavy chain FR regions contained in SEQ ID NO: 162 and light chain FR regions contained in SEQ ID NO: 127. In some embodiments, the antibody or antigen binding fragment thereof comprises heavy chain FR regions contained in SEQ ID NO: 162 and light chain FR regions contained in SEQ ID NO: 128. In some embodiments, the antibody or antigen binding fragment thereof comprises heavy chain FR regions contained in SEQ ID NO: 162 and light chain FR regions contained in SEQ ID NO: 129. In some embodiments, the antibody or antigen binding fragment thereof comprises heavy chain FR regions contained in SEQ ID NO: 162 and light chain FR regions contained in SEQ ID NO: 130. In some embodiments, the antibody or antigen binding fragment thereof comprises heavy chain FR regions contained in SEQ ID NO: 162 and light chain FR regions contained in SEQ ID NO: 131. In some embodiments, the antibody or antigen binding fragment thereof comprises heavy chain FR regions contained in SEQ ID NO: 162 and light chain FR regions contained in SEQ ID NO: 132. In some embodiments, the antibody or antigen binding fragment thereof comprises heavy chain FR regions contained in SEQ ID NO: 162 and light chain FR regions contained in SEQ ID NO: 133. In some embodiments, the antibody or antigen binding fragment thereof comprises heavy chain FR regions contained in SEQ ID NO: 162 and light chain FR regions contained in SEQ ID NO: 134. In some embodiments, the antibody or antigen binding fragment thereof comprises heavy chain FR regions contained in SEQ ID NO: 162 and light chain FR regions contained in SEQ ID NO: 135. In some embodiments, the antibody or antigen binding fragment thereof comprises heavy chain FR regions contained in SEQ ID NO: 162 and light chain FR regions contained in SEQ ID NO: 136. In some embodiments, the antibody or antigen binding fragment thereof comprises heavy chain FR regions contained in SEQ ID NO: 162 and light chain FR regions contained in SEQ ID NO: 137. In some embodiments, the antibody or antigen binding fragment thereof comprises heavy chain FR regions contained in SEQ ID NO: 162 and light chain FR regions contained in SEQ ID NO: 138.

In some embodiments, the antibody or antigen binding fragment thereof comprises heavy chain FR regions contained in SEQ ID NO: 163 and light chain FR regions contained in SEQ ID NO: 114. In some embodiments, the antibody or antigen binding fragment thereof comprises heavy chain FR regions contained in SEQ ID NO: 163 and light chain FR regions contained in SEQ ID NO: 116. In some embodiments, the antibody or antigen binding fragment thereof comprises heavy chain FR regions contained in SEQ ID NO: 163 and light chain FR regions contained in SEQ ID NO: 117. In some embodiments, the antibody or antigen binding fragment thereof comprises heavy chain FR regions contained in SEQ ID NO: 163 and light chain FR regions contained in SEQ ID NO: 118. In some embodiments, the antibody or antigen binding fragment thereof comprises heavy chain FR regions contained in SEQ ID NO: 163 and light chain FR regions contained in SEQ ID NO: 119. In some embodiments, the antibody or antigen binding fragment thereof comprises heavy chain FR regions contained in SEQ ID NO: 163 and light chain FR regions contained in SEQ ID NO: 120. In some embodiments, the antibody or antigen binding fragment thereof comprises heavy chain FR regions contained in SEQ ID NO: 163 and light chain FR regions contained in SEQ ID NO: 121. In some embodiments, the antibody or antigen binding fragment thereof comprises heavy chain FR regions contained in SEQ ID NO: 163 and light chain FR regions contained in SEQ ID NO: 122. In some embodiments, the antibody or antigen binding fragment thereof comprises heavy chain FR regions contained in SEQ ID NO: 163 and light chain FR regions contained in SEQ ID NO: 123. In some embodiments, the antibody or antigen binding fragment thereof comprises heavy chain FR regions contained in SEQ ID NO: 163 and light chain FR regions contained in SEQ ID NO: 124. In some embodiments, the antibody or antigen binding fragment thereof comprises heavy chain FR regions contained in SEQ ID NO: 163 and light chain FR regions contained in SEQ ID NO: 125. In some embodiments, the antibody or antigen binding fragment thereof comprises heavy chain FR regions contained in SEQ ID NO: 163 and light chain FR regions contained in SEQ ID NO: 126. In some embodiments, the antibody or antigen binding fragment thereof comprises heavy chain FR regions contained in SEQ ID NO: 163 and light chain FR regions contained in SEQ ID NO: 127. In some embodiments, the antibody or antigen binding fragment thereof comprises heavy chain FR regions contained in SEQ ID NO: 163 and light chain FR regions contained in SEQ ID NO: 128. In some embodiments, the antibody or antigen binding fragment thereof comprises heavy chain FR regions contained in SEQ ID NO: 163 and light chain FR regions contained in SEQ ID NO: 129. In some embodiments, the antibody or antigen binding fragment thereof comprises heavy chain FR regions contained in SEQ ID NO: 163 and light chain FR regions contained in SEQ ID NO: 130. In some embodiments, the antibody or antigen binding fragment thereof comprises heavy chain FR regions contained in SEQ ID NO: 163 and light chain FR regions contained in SEQ ID NO: 131. In some embodiments, the antibody or antigen binding fragment thereof comprises heavy chain FR regions contained in SEQ ID NO: 163 and light chain FR regions contained in SEQ ID NO: 132. In some embodiments, the antibody or antigen binding fragment thereof comprises heavy chain FR regions contained in SEQ ID NO: 163 and light chain FR regions contained in SEQ ID NO: 133. In some embodiments, the antibody or antigen binding fragment thereof comprises heavy chain FR regions contained in SEQ ID NO: 163 and light chain FR regions contained in SEQ ID NO: 134. In some embodiments, the antibody or antigen binding fragment thereof comprises heavy chain FR regions contained in SEQ ID NO: 163 and light chain FR regions contained in SEQ ID NO: 135. In some embodiments, the antibody or antigen binding fragment thereof comprises heavy chain FR regions contained in SEQ ID NO: 163 and light chain FR regions contained in SEQ ID NO: 136. In some embodiments, the antibody or antigen binding fragment thereof comprises heavy chain FR regions contained in SEQ ID NO: 163 and light chain FR regions contained in SEQ ID NO: 137. In some embodiments, the antibody or antigen binding fragment thereof comprises heavy chain FR regions contained in SEQ ID NO: 163 and light chain FR regions contained in SEQ ID NO: 138.

In other embodiments, the antibody or antigen binding fragment thereof comprises heavy chain FR regions contained in SEQ ID NOs: 165 and 171-177, and/or light chain FR regions contained in SEQ ID NOs: 164 and 166-170.

In some embodiments, the antibody or antigen binding fragment thereof comprises heavy chain FR regions contained in SEQ ID NO: 165 and light chain FR regions contained in SEQ ID NO: 164. In some embodiments, the antibody or antigen binding fragment thereof comprises heavy chain FR regions contained in SEQ ID NO: 165 and light chain FR regions contained in SEQ ID NO: 166. In some embodiments, the antibody or antigen binding fragment thereof comprises heavy chain FR regions contained in SEQ ID NO: 165 and light chain FR regions contained in SEQ ID NO: 167. In some embodiments, the antibody or antigen binding fragment thereof comprises heavy chain FR regions contained in SEQ ID NO: 165 and light chain FR regions contained in SEQ ID NO: 168. In some embodiments, the antibody or antigen binding fragment thereof comprises heavy chain FR regions contained in SEQ ID NO: 165 and light chain FR regions contained in SEQ ID NO: 169. In some embodiments, the antibody or antigen binding fragment thereof comprises heavy chain FR regions contained in SEQ ID NO: 165 and light chain FR regions contained in SEQ ID NO: 170.

In some embodiments, the antibody or antigen binding fragment thereof comprises heavy chain FR regions contained in SEQ ID NO: 171 and light chain FR regions contained in SEQ ID NO: 164. In some embodiments, the antibody or antigen binding fragment thereof comprises heavy chain FR regions contained in SEQ ID NO: 171 and light chain FR regions contained in SEQ ID NO: 166. In some embodiments, the antibody or antigen binding fragment thereof comprises heavy chain FR regions contained in SEQ ID NO: 171 and light chain FR regions contained in SEQ ID NO: 167. In some embodiments, the antibody or antigen binding fragment thereof comprises heavy chain FR regions contained in SEQ ID NO: 171 and light chain FR regions contained in SEQ ID NO: 168. In some embodiments, the antibody or antigen binding fragment thereof comprises heavy chain FR regions contained in SEQ ID NO: 171 and light chain FR regions contained in SEQ ID NO: 169. In some embodiments, the antibody or antigen binding fragment thereof comprises heavy chain FR regions contained in SEQ ID NO: 171 and light chain FR regions contained in SEQ ID NO: 170.

In some embodiments, the antibody or antigen binding fragment thereof comprises heavy chain FR regions contained in SEQ ID NO: 172 and light chain FR regions contained in SEQ ID NO: 164. In some embodiments, the antibody or antigen binding fragment thereof comprises heavy chain FR regions contained in SEQ ID NO: 172 and light chain FR regions contained in SEQ ID NO: 166. In some embodiments, the antibody or antigen binding fragment thereof comprises heavy chain FR regions contained in SEQ ID NO: 172 and light chain FR regions contained in SEQ ID NO: 167. In some embodiments, the antibody or antigen binding fragment thereof comprises heavy chain FR regions contained in SEQ ID NO: 172 and light chain FR regions contained in SEQ ID NO: 168. In some embodiments, the antibody or antigen binding fragment thereof comprises heavy chain FR regions contained in SEQ ID NO: 172 and light chain FR regions contained in SEQ ID NO: 169. In some embodiments, the antibody or antigen binding fragment thereof comprises heavy chain FR regions contained in SEQ ID NO: 172 and light chain FR regions contained in SEQ ID NO: 170.

In some embodiments, the antibody or antigen binding fragment thereof comprises heavy chain FR regions contained in SEQ ID NO: 173 and light chain FR regions contained in SEQ ID NO: 164. In some embodiments, the antibody or antigen binding fragment thereof comprises heavy chain FR regions contained in SEQ ID NO: 173 and light chain FR regions contained in SEQ ID NO: 166. In some embodiments, the antibody or antigen binding fragment thereof comprises heavy chain FR regions contained in SEQ ID NO: 173 and light chain FR regions contained in SEQ ID NO: 167. In some embodiments, the antibody or antigen binding fragment thereof comprises heavy chain FR regions contained in SEQ ID NO: 173 and light chain FR regions contained in SEQ ID NO: 168. In some embodiments, the antibody or antigen binding fragment thereof comprises heavy chain FR regions contained in SEQ ID NO: 173 and light chain FR regions contained in SEQ ID NO: 169. In some embodiments, the antibody or antigen binding fragment thereof comprises heavy chain FR regions contained in SEQ ID NO: 173 and light chain FR regions contained in SEQ ID NO: 170.

In some embodiments, the antibody or antigen binding fragment thereof comprises heavy chain FR regions contained in SEQ ID NO: 174 and light chain FR regions contained in SEQ ID NO: 164. In some embodiments, the antibody or antigen binding fragment thereof comprises heavy chain FR regions contained in SEQ ID NO: 174 and light chain FR regions contained in SEQ ID NO: 166. In some embodiments, the antibody or antigen binding fragment thereof comprises heavy chain FR regions contained in SEQ ID NO: 174 and light chain FR regions contained in SEQ ID NO: 167. In some embodiments, the antibody or antigen binding fragment thereof comprises heavy chain FR regions contained in SEQ ID NO: 174 and light chain FR regions contained in SEQ ID NO: 168. In some embodiments, the antibody or antigen binding fragment thereof comprises heavy chain FR regions contained in SEQ ID NO: 174 and light chain FR regions contained in SEQ ID NO: 169. In some embodiments, the antibody or antigen binding fragment thereof comprises heavy chain FR regions contained in SEQ ID NO: 174 and light chain FR regions contained in SEQ ID NO: 170.

In some embodiments, the antibody or antigen binding fragment thereof comprises heavy chain FR regions contained in SEQ ID NO: 175 and light chain FR regions contained in SEQ ID NO: 164. In some embodiments, the antibody or antigen binding fragment thereof comprises heavy chain FR regions contained in SEQ ID NO: 175 and light chain FR regions contained in SEQ ID NO: 166. In some embodiments, the antibody or antigen binding fragment thereof comprises heavy chain FR regions contained in SEQ ID NO: 175 and light chain FR regions contained in SEQ ID NO: 167. In some embodiments, the antibody or antigen binding fragment thereof comprises heavy chain FR regions contained in SEQ ID NO: 175 and light chain FR regions contained in SEQ ID NO: 168. In some embodiments, the antibody or antigen binding fragment thereof comprises heavy chain FR regions contained in SEQ ID NO: 175 and light chain FR regions contained in SEQ ID NO: 169. In some embodiments, the antibody or antigen binding fragment thereof comprises heavy chain FR regions contained in SEQ ID NO: 175 and light chain FR regions contained in SEQ ID NO: 170.

In some embodiments, the antibody or antigen binding fragment thereof comprises heavy chain FR regions contained in SEQ ID NO: 176 and light chain FR regions contained in SEQ ID NO: 164. In some embodiments, the antibody or antigen binding fragment thereof comprises heavy chain FR regions contained in SEQ ID NO: 176 and light chain FR regions contained in SEQ ID NO: 166. In some embodiments, the antibody or antigen binding fragment thereof comprises heavy chain FR regions contained in SEQ ID NO: 176 and light chain FR regions contained in SEQ ID NO: 167. In some embodiments, the antibody or antigen binding fragment thereof comprises heavy chain FR regions contained in SEQ ID NO: 176 and light chain FR regions contained in SEQ ID NO: 168. In some embodiments, the antibody or antigen binding fragment thereof comprises heavy chain FR regions contained in SEQ ID NO: 176 and light chain FR regions contained in SEQ ID NO: 169. In some embodiments, the antibody or antigen binding fragment thereof comprises heavy chain FR regions contained in SEQ ID NO: 176 and light chain FR regions contained in SEQ ID NO: 170.

In some embodiments, the antibody or antigen binding fragment thereof comprises heavy chain FR regions contained in SEQ ID NO: 177 and light chain FR regions contained in SEQ ID NO: 164. In some embodiments, the antibody or antigen binding fragment thereof comprises heavy chain FR regions contained in SEQ ID NO: 177 and light chain FR regions contained in SEQ ID NO: 166. In some embodiments, the antibody or antigen binding fragment thereof comprises heavy chain FR regions contained in SEQ ID NO: 177 and light chain FR regions contained in SEQ ID NO: 167. In some embodiments, the antibody or antigen binding fragment thereof comprises heavy chain FR regions contained in SEQ ID NO: 177 and light chain FR regions contained in SEQ ID NO: 168. In some embodiments, the antibody or antigen binding fragment thereof comprises heavy chain FR regions contained in SEQ ID NO: 177 and light chain FR regions contained in SEQ ID NO: 169. In some embodiments, the antibody or antigen binding fragment thereof comprises heavy chain FR regions contained in SEQ ID NO: 177 and light chain FR regions contained in SEQ ID NO: 170.

Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In certain embodiments, the antibody or antigen binding fragments provided herein comprises VH and/ VL regions of antibody 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c.

In some embodiments, the antibody or antigen binding fragment provided herein comprises a VH region listed in Table 8. In some embodiments, the antibody or antigen binding fragment provided herein comprises a VL region listed Table 10.

In some embodiments, the antibody or antigen binding fragment thereof comprises a VH region comprising an amino acid sequence of SEQ ID NO: 23. In some embodiments, the antibody or antigen binding fragment thereof comprises a VL region comprising an amino acid sequence of SEQ ID NO: 51. In some embodiments, the antibody or antigen binding fragment thereof comprises a VH region comprising an amino acid sequence of SEQ ID NO: 23, and a VL region comprising an amino acid sequence of SEQ ID NO: 51.

In some embodiments, the antibody or antigen binding fragment thereof comprises a VH region comprising an amino acid sequence of SEQ ID NO: 27. In some embodiments, the antibody or antigen binding fragment thereof comprises a VL region comprising an amino acid sequence of SEQ ID NO: 55. In some embodiments, the antibody or antigen binding fragment thereof comprises a VH region comprising an amino acid sequence of SEQ ID NO: 27, and a VL region comprising an amino acid sequence of SEQ ID NO: 55.

In some embodiments, the antibody or antigen binding fragment thereof comprises a VH region comprising an amino acid sequence of SEQ ID NO: 31. In some embodiments, the antibody or antigen binding fragment thereof comprises a VL region comprising an amino acid sequence of SEQ ID NO: 55. In some embodiments, the antibody or antigen binding fragment thereof comprises a VH region comprising an amino acid sequence of SEQ ID NO: 31, and a VL region comprising an amino acid sequence of SEQ ID NO: 55.

In some embodiments, the antibody or antigen binding fragment thereof comprises a VH region comprising an amino acid sequence of SEQ ID NO: 35. In some embodiments, the antibody or antigen binding fragment thereof comprises a VL region comprising an amino acid sequence of SEQ ID NO: 55. In some embodiments, the antibody or antigen binding fragment thereof comprises a VH region comprising an amino acid sequence of SEQ ID NO: 35, and a VL region comprising an amino acid sequence of SEQ ID NO: 55.

In some embodiments, the antibody or antigen binding fragment thereof comprises a VH region comprising an amino acid sequence of SEQ ID NO: 39. In some embodiments, the antibody or antigen binding fragment thereof comprises a VL region comprising an amino acid sequence of SEQ ID NO: 59. In some embodiments, the antibody or antigen binding fragment thereof comprises a VH region comprising an amino acid sequence of SEQ ID NO: 39, and a VL region comprising an amino acid sequence of SEQ ID NO: 59.

In some embodiments, the antibody or antigen binding fragment thereof comprises a VH region comprising an amino acid sequence of SEQ ID NO: 43. In some embodiments, the antibody or antigen binding fragment thereof comprises a VL region comprising an amino acid sequence of SEQ ID NO: 63. In some embodiments, the antibody or antigen binding fragment thereof comprises a VH region comprising an amino acid sequence of SEQ ID NO: 43, and a VL region comprising an amino acid sequence of SEQ ID NO: 63.

In some embodiments, the antibody or antigen binding fragment thereof comprises a VH region comprising an amino acid sequence of SEQ ID NO: 47. In some embodiments, the antibody or antigen binding fragment thereof comprises a VL region comprising an amino acid sequence of SEQ ID NO: 67. In some embodiments, the antibody or antigen binding fragment thereof comprises a VH region comprising an amino acid sequence of SEQ ID NO: 47, and a VL region comprising an amino acid sequence of SEQ ID NO: 67.

In other embodiments, the antibody or antigen binding fragment thereof provided herein comprises a VH and/or a VL of the hunaminzed antibodies described in Section 6 below and/or in FIGS. 10-13.

In some embodiments, the antibody or antigen binding fragment thereof comprises a VH having a sequence selected from SEQ ID NOs: 115, 139-163, 165, and 171-177, and/or a VL having a sequence selected from SEQ ID NOs: 114, 116-138, 164, and 166-170.

In some embodiments, the antibody or antigen binding fragment thereof comprises a VH having a sequence selected from SEQ ID NOs: 115 and 139-163, and/or a VL having a sequence selected from SEQ ID NOs: 114 and 116-138.

In some embodiments, the antibody or antigen binding fragment thereof comprises a VH of SEQ ID NO: 115 and a VL of SEQ ID NO: 114. In some embodiments, the antibody or antigen binding fragment thereof comprises a VH of SEQ ID NO: 115 and a VL of SEQ ID NO: 116. In some embodiments, the antibody or antigen binding fragment thereof comprises a VH of SEQ ID NO: 115 and a VL of SEQ ID NO: 117. In some embodiments, the antibody or antigen binding fragment thereof comprises a VH of SEQ ID NO: 115 and a VL of SEQ ID NO: 118. In some embodiments, the antibody or antigen binding fragment thereof comprises a VH of SEQ ID NO: 115 and a VL of SEQ ID NO: 119. In some embodiments, the antibody or antigen binding fragment thereof comprises a VH of SEQ ID NO: 115 and a VL of SEQ ID NO: 120. In some embodiments, the antibody or antigen binding fragment thereof comprises a VH of SEQ ID NO: 115 and a VL of SEQ ID NO: 121. In some embodiments, the antibody or antigen binding fragment thereof comprises a VH of SEQ ID NO: 115 and a VL of SEQ ID NO: 122. In some embodiments, the antibody or antigen binding fragment thereof comprises a VH of SEQ ID NO: 115 and a VL of SEQ ID NO: 123. In some embodiments, the antibody or antigen binding fragment thereof comprises a VH of SEQ ID NO: 115 and a VL of SEQ ID NO: 124. In some embodiments, the antibody or antigen binding fragment thereof comprises a VH of SEQ ID NO: 115 and a VL of SEQ ID NO: 125. In some embodiments, the antibody or antigen binding fragment thereof comprises a VH of SEQ ID NO: 115 and a VL of SEQ ID NO: 126. In some embodiments, the antibody or antigen binding fragment thereof comprises a VH of SEQ ID NO: 115 and a VL of SEQ ID NO: 127. In some embodiments, the antibody or antigen binding fragment thereof comprises a VH of SEQ ID NO: 115 and a VL of SEQ ID NO: 128. In some embodiments, the antibody or antigen binding fragment thereof comprises a VH of SEQ ID NO: 115 and a VL of SEQ ID NO: 129. In some embodiments, the antibody or antigen binding fragment thereof comprises a VH of SEQ ID NO: 115 and a VL of SEQ ID NO: 130. In some embodiments, the antibody or antigen binding fragment thereof comprises a VH of SEQ ID NO: 115 and a VL of SEQ ID NO: 131. In some embodiments, the antibody or antigen binding fragment thereof comprises a VH of SEQ ID NO: 115 and a VL of SEQ ID NO: 132. In some embodiments, the antibody or antigen binding fragment thereof comprises a VH of SEQ ID NO: 115 and a VL of SEQ ID NO: 133. In some embodiments, the antibody or antigen binding fragment thereof comprises a VH of SEQ ID NO: 115 and a VL of SEQ ID NO: 134. In some embodiments, the antibody or antigen binding fragment thereof comprises a VH of SEQ ID NO: 115 and a VL of SEQ ID NO: 135. In some embodiments, the antibody or antigen binding fragment thereof comprises a VH of SEQ ID NO: 115 and a VL of SEQ ID NO: 136. In some embodiments, the antibody or antigen binding fragment thereof comprises a VH of SEQ ID NO: 115 and a VL of SEQ ID NO: 137. In some embodiments, the antibody or antigen binding fragment thereof comprises a VH of SEQ ID NO: 115 and a VL of SEQ ID NO: 138.

In some embodiments, the antibody or antigen binding fragment thereof comprises a VH of SEQ ID NO: 139 and a VL of SEQ ID NO: 114. In some embodiments, the antibody or antigen binding fragment thereof comprises a VH of SEQ ID NO: 139 and a VL of SEQ ID NO: 116. In some embodiments, the antibody or antigen binding fragment thereof comprises a VH of SEQ ID NO: 139 and a VL of SEQ ID NO: 117. In some embodiments, the antibody or antigen binding fragment thereof comprises a VH of SEQ ID NO: 139 and a VL of SEQ ID NO: 118. In some embodiments, the antibody or antigen binding fragment thereof comprises a VH of SEQ ID NO: 139 and a VL of SEQ ID NO: 119. In some embodiments, the antibody or antigen binding fragment thereof comprises a VH of SEQ ID NO: 139 and a VL of SEQ ID NO: 120. In some embodiments, the antibody or antigen binding fragment thereof comprises a VH of SEQ ID NO: 139 and a VL of SEQ ID NO: 121. In some embodiments, the antibody or antigen binding fragment thereof comprises a VH of SEQ ID NO: 139 and a VL of SEQ ID NO: 122. In some embodiments, the antibody or antigen binding fragment thereof comprises a VH of SEQ ID NO: 139 and a VL of SEQ ID NO: 123. In some embodiments, the antibody or antigen binding fragment thereof comprises a VH of SEQ ID NO: 139 and a VL of SEQ ID NO: 124. In some embodiments, the antibody or antigen binding fragment thereof comprises a VH of SEQ ID NO: 139 and a VL of SEQ ID NO: 125. In some embodiments, the antibody or antigen binding fragment thereof comprises a VH of SEQ ID NO: 139 and a VL of SEQ ID NO: 126. In some embodiments, the antibody or antigen binding fragment thereof comprises a VH of SEQ ID NO: 139 and a VL of SEQ ID NO: 127. In some embodiments, the antibody or antigen binding fragment thereof comprises a VH of SEQ ID NO: 139 and a VL of SEQ ID NO: 128. In some embodiments, the antibody or antigen binding fragment thereof comprises a VH of SEQ ID NO: 139 and a VL of SEQ ID NO: 129. In some embodiments, the antibody or antigen binding fragment thereof comprises a VH of SEQ ID NO: 139 and a VL of SEQ ID NO: 130. In some embodiments, the antibody or antigen binding fragment thereof comprises a VH of SEQ ID NO: 139 and a VL of SEQ ID NO: 131. In some embodiments, the antibody or antigen binding fragment thereof comprises a VH of SEQ ID NO: 139 and a VL of SEQ ID NO: 132. In some embodiments, the antibody or antigen binding fragment thereof comprises a VH of SEQ ID NO: 139 and a VL of SEQ ID NO: 133. In some embodiments, the antibody or antigen binding fragment thereof comprises a VH of SEQ ID NO: 139 and a VL of SEQ ID NO: 134. In some embodiments, the antibody or antigen binding fragment thereof comprises a VH of SEQ ID NO: 139 and a VL of SEQ ID NO: 135. In some embodiments, the antibody or antigen binding fragment thereof comprises a VH of SEQ ID NO: 139 and a VL of SEQ ID NO: 136. In some embodiments, the antibody or antigen binding fragment thereof comprises a VH of SEQ ID NO: 139 and a VL of SEQ ID NO: 137. In some embodiments, the antibody or antigen binding fragment thereof comprises a VH of SEQ ID NO: 139 and a VL of SEQ ID NO: 138.

In some embodiments, the antibody or antigen binding fragment thereof comprises a VH of SEQ ID NO: 140 and a VL of SEQ ID NO: 114. In some embodiments, the antibody or antigen binding fragment thereof comprises a VH of SEQ ID NO: 140 and a VL of SEQ ID NO: 116. In some embodiments, the antibody or antigen binding fragment thereof comprises a VH of SEQ ID NO: 140 and a VL of SEQ ID NO: 117. In some embodiments, the antibody or antigen binding fragment thereof comprises a VH of SEQ ID NO: 140 and a VL of SEQ ID NO: 118. In some embodiments, the antibody or antigen binding fragment thereof comprises a VH of SEQ ID NO: 140 and a VL of SEQ ID NO: 119. In some embodiments, the antibody or antigen binding fragment thereof comprises a VH of SEQ ID NO: 140 and a VL of SEQ ID NO: 120. In some embodiments, the antibody or antigen binding fragment thereof comprises a VH of SEQ ID NO: 140 and a VL of SEQ ID NO: 121. In some embodiments, the antibody or antigen binding fragment thereof comprises a VH of SEQ ID NO: 140 and a VL of SEQ ID NO: 122. In some embodiments, the antibody or antigen binding fragment thereof comprises a VH of SEQ ID NO: 140 and a VL of SEQ ID NO: 123. In some embodiments, the antibody or antigen binding fragment thereof comprises a VH of SEQ ID NO: 140 and a VL of SEQ ID NO: 124. In some embodiments, the antibody or antigen binding fragment thereof comprises a VH of SEQ ID NO: 140 and a VL of SEQ ID NO: 125. In some embodiments, the antibody or antigen binding fragment thereof comprises a VH of SEQ ID NO: 140 and a VL of SEQ ID NO: 126. In some embodiments, the antibody or antigen binding fragment thereof comprises a VH of SEQ ID NO: 140 and a VL of SEQ ID NO: 127. In some embodiments, the antibody or antigen binding fragment thereof comprises a VH of SEQ ID NO: 140 and a VL of SEQ ID NO: 128. In some embodiments, the antibody or antigen binding fragment thereof comprises a VH of SEQ ID NO: 140 and a VL of SEQ ID NO: 129. In some embodiments, the antibody or antigen binding fragment thereof comprises a VH of SEQ ID NO: 140 and a VL of SEQ ID NO: 130. In some embodiments, the antibody or antigen binding fragment thereof comprises a VH of SEQ ID NO: 140 and a VL of SEQ ID NO: 131. In some embodiments, the antibody or antigen binding fragment thereof comprises a VH of SEQ ID NO: 140 and a VL of SEQ ID NO: 132. In some embodiments, the antibody or antigen binding fragment thereof comprises a VH of SEQ ID NO: 140 and a VL of SEQ ID NO: 133. In some embodiments, the antibody or antigen binding fragment thereof comprises a VH of SEQ ID NO: 140 and a VL of SEQ ID NO: 134. In some embodiments, the antibody or antigen binding fragment thereof comprises a VH of SEQ ID NO: 140 and a VL of SEQ ID NO: 135. In some embodiments, the antibody or antigen binding fragment thereof comprises a VH of SEQ ID NO: 140 and a VL of SEQ ID NO: 136. In some embodiments, the antibody or antigen binding fragment thereof comprises a VH of SEQ ID NO: 140 and a VL of SEQ ID NO: 137. In some embodiments, the antibody or antigen binding fragment thereof comprises a VH of SEQ ID NO: 140 and a VL of SEQ ID NO: 138.

In some embodiments, the antibody or antigen binding fragment thereof comprises a VH of SEQ ID NO: 141 and a VL of SEQ ID NO: 114. In some embodiments, the antibody or antigen binding fragment thereof comprises a VH of SEQ ID NO: 141 and a VL of SEQ ID NO: 116. In some embodiments, the antibody or antigen binding fragment thereof comprises a VH of SEQ ID NO: 141 and a VL of SEQ ID NO: 117. In some embodiments, the antibody or antigen binding fragment thereof comprises a VH of SEQ ID NO: 141 and a VL of SEQ ID NO: 118. In some embodiments, the antibody or antigen binding fragment thereof comprises a VH of SEQ ID NO: 141 and a VL of SEQ ID NO: 119. In some embodiments, the antibody or antigen binding fragment thereof comprises a VH of SEQ ID NO: 141 and a VL of SEQ ID NO: 120. In some embodiments, the antibody or antigen binding fragment thereof comprises a VH of SEQ ID NO: 141 and a VL of SEQ ID NO: 121. In some embodiments, the antibody or antigen binding fragment thereof comprises a VH of SEQ ID NO: 141 and a VL of SEQ ID NO: 122. In some embodiments, the antibody or antigen binding fragment thereof comprises a VH of SEQ ID NO: 141 and a VL of SEQ ID NO: 123. In some embodiments, the antibody or antigen binding fragment thereof comprises a VH of SEQ ID NO: 141 and a VL of SEQ ID NO: 124. In some embodiments, the antibody or antigen binding fragment thereof comprises a VH of SEQ ID NO: 141 and a VL of SEQ ID NO: 125. In some embodiments, the antibody or antigen binding fragment thereof comprises a VH of SEQ ID NO: 141 and a VL of SEQ ID NO: 126. In some embodiments, the antibody or antigen binding fragment thereof comprises a VH of SEQ ID NO: 141 and a VL of SEQ ID NO: 127. In some embodiments, the antibody or antigen binding fragment thereof comprises a VH of SEQ ID NO: 141 and a VL of SEQ ID NO: 128. In some embodiments, the antibody or antigen binding fragment thereof comprises a VH of SEQ ID NO: 141 and a VL of SEQ ID NO: 129. In some embodiments, the antibody or antigen binding fragment thereof comprises a VH of SEQ ID NO: 141 and a VL of SEQ ID NO: 130. In some embodiments, the antibody or antigen binding fragment thereof comprises a VH of SEQ ID NO: 141 and a VL of SEQ ID NO: 131. In some embodiments, the antibody or antigen binding fragment thereof comprises a VH of SEQ ID NO: 141 and a VL of SEQ ID NO: 132. In some embodiments, the antibody or antigen binding fragment thereof comprises a VH of SEQ ID NO: 141 and a VL of SEQ ID NO: 133. In some embodiments, the antibody or antigen binding fragment thereof comprises a VH of SEQ ID NO: 141 and a VL of SEQ ID NO: 134. In some embodiments, the antibody or antigen binding fragment thereof comprises a VH of SEQ ID NO: 141 and a VL of SEQ ID NO: 135. In some embodiments, the antibody or antigen binding fragment thereof comprises a VH of SEQ ID NO: 141 and a VL of SEQ ID NO: 136. In some embodiments, the antibody or antigen binding fragment thereof comprises a VH of SEQ ID NO: 141 and a VL of SEQ ID NO: 137. In some embodiments, the antibody or antigen binding fragment thereof comprises a VH of SEQ ID NO: 141 and a VL of SEQ ID NO: 138.

In some embodiments, the antibody or antigen binding fragment thereof comprises a VH of SEQ ID NO: 142 and a VL of SEQ ID NO: 114. In some embodiments, the antibody or antigen binding fragment thereof comprises a VH of SEQ ID NO: 142 and a VL of SEQ ID NO: 116. In some embodiments, the antibody or antigen binding fragment thereof comprises a VH of SEQ ID NO: 142 and a VL of SEQ ID NO: 117. In some embodiments, the antibody or antigen binding fragment thereof comprises a VH of SEQ ID NO: 142 and a VL of SEQ ID NO: 118. In some embodiments, the antibody or antigen binding fragment thereof comprises a VH of SEQ ID NO: 142 and a VL of SEQ ID NO: 119. In some embodiments, the antibody or antigen binding fragment thereof comprises a VH of SEQ ID NO: 142 and a VL of SEQ ID NO: 120. In some embodiments, the antibody or antigen binding fragment thereof comprises a VH of SEQ ID NO: 142 and a VL of SEQ ID NO: 121. In some embodiments, the antibody or antigen binding fragment thereof comprises a VH of SEQ ID NO: 142 and a VL of SEQ ID NO: 122. In some embodiments, the antibody or antigen binding fragment thereof comprises a VH of SEQ ID NO: 142 and a VL of SEQ ID NO: 123. In some embodiments, the antibody or antigen binding fragment thereof comprises a VH of SEQ ID NO: 142 and a VL of SEQ ID NO: 124. In some embodiments, the antibody or antigen binding fragment thereof comprises a VH of SEQ ID NO: 142 and a VL of SEQ ID NO: 125. In some embodiments, the antibody or antigen binding fragment thereof comprises a VH of SEQ ID NO: 142 and a VL of SEQ ID NO: 126. In some embodiments, the antibody or antigen binding fragment thereof comprises a VH of SEQ ID NO: 142 and a VL of SEQ ID NO: 127. In some embodiments, the antibody or antigen binding fragment thereof comprises a VH of SEQ ID NO: 142 and a VL of SEQ ID NO: 128. In some embodiments, the antibody or antigen binding fragment thereof comprises a VH of SEQ ID NO: 142 and a VL of SEQ ID NO: 129. In some embodiments, the antibody or antigen binding fragment thereof comprises a VH of SEQ ID NO: 142 and a VL of SEQ ID NO: 130. In some embodiments, the antibody or antigen binding fragment thereof comprises a VH of SEQ ID NO: 142 and a VL of SEQ ID NO: 131. In some embodiments, the antibody or antigen binding fragment thereof comprises a VH of SEQ ID NO: 142 and a VL of SEQ ID NO: 132. In some embodiments, the antibody or antigen binding fragment thereof comprises a VH of SEQ ID NO: 142 and a VL of SEQ ID NO: 133. In some embodiments, the antibody or antigen binding fragment thereof comprises a VH of SEQ ID NO: 142 and a VL of SEQ ID NO: 134. In some embodiments, the antibody or antigen binding fragment thereof comprises a VH of SEQ ID NO: 142 and a VL of SEQ ID NO: 135. In some embodiments, the antibody or antigen binding fragment thereof comprises a VH of SEQ ID NO: 142 and a VL of SEQ ID NO: 136. In some embodiments, the antibody or antigen binding fragment thereof comprises a VH of SEQ ID NO: 142 and a VL of SEQ ID NO: 137. In some embodiments, the antibody or antigen binding fragment thereof comprises a VH of SEQ ID NO: 142 and a VL of SEQ ID NO: 138.

In some embodiments, the antibody or antigen binding fragment thereof comprises a VH of SEQ ID NO: 143 and a VL of SEQ ID NO: 114. In some embodiments, the antibody or antigen binding fragment thereof comprises a VH of SEQ ID NO: 143 and a VL of SEQ ID NO: 116. In some embodiments, the antibody or antigen binding fragment thereof comprises a VH of SEQ ID NO: 143 and a VL of SEQ ID NO: 117. In some embodiments, the antibody or antigen binding fragment thereof comprises a VH of SEQ ID NO: 143 and a VL of SEQ ID NO: 118. In some embodiments, the antibody or antigen binding fragment thereof comprises a VH of SEQ ID NO: 143 and a VL of SEQ ID NO: 119. In some embodiments, the antibody or antigen binding fragment thereof comprises a VH of SEQ ID NO: 143 and a VL of SEQ ID NO: 120. In some embodiments, the antibody or antigen binding fragment thereof comprises a VH of SEQ ID NO: 143 and a VL of SEQ ID NO: 121. In some embodiments, the antibody or antigen binding fragment thereof comprises a VH of SEQ ID NO: 143 and a VL of SEQ ID NO: 122. In some embodiments, the antibody or antigen binding fragment thereof comprises a VH of SEQ ID NO: 143 and a VL of SEQ ID NO: 123. In some embodiments, the antibody or antigen binding fragment thereof comprises a VH of SEQ ID NO: 143 and a VL of SEQ ID NO: 124. In some embodiments, the antibody or antigen binding fragment thereof comprises a VH of SEQ ID NO: 143 and a VL of SEQ ID NO: 125. In some embodiments, the antibody or antigen binding fragment thereof comprises a VH of SEQ ID NO: 143 and a VL of SEQ ID NO: 126. In some embodiments, the antibody or antigen binding fragment thereof comprises a VH of SEQ ID NO: 143 and a VL of SEQ ID NO: 127. In some embodiments, the antibody or antigen binding fragment thereof comprises a VH of SEQ ID NO: 143 and a VL of SEQ ID NO: 128. In some embodiments, the antibody or antigen binding fragment thereof comprises a VH of SEQ ID NO: 143 and a VL of SEQ ID NO: 129. In some embodiments, the antibody or antigen binding fragment thereof comprises a VH of SEQ ID NO: 143 and a VL of SEQ ID NO: 130. In some embodiments, the antibody or antigen binding fragment thereof comprises a VH of SEQ ID NO: 143 and a VL of SEQ ID NO: 131. In some embodiments, the antibody or antigen binding fragment thereof comprises a VH of SEQ ID NO: 143 and a VL of SEQ ID NO: 132. In some embodiments, the antibody or antigen binding fragment thereof comprises a VH of SEQ ID NO: 143 and a VL of SEQ ID NO: 133. In some embodiments, the antibody or antigen binding fragment thereof comprises a VH of SEQ ID NO: 143 and a VL of SEQ ID NO: 134. In some embodiments, the antibody or antigen binding fragment thereof comprises a VH of SEQ ID NO: 143 and a VL of SEQ ID NO: 135. In some embodiments, the antibody or antigen binding fragment thereof comprises a VH of SEQ ID NO: 143 and a VL of SEQ ID NO: 136. In some embodiments, the antibody or antigen binding fragment thereof comprises a VH of SEQ ID NO: 143 and a VL of SEQ ID NO: 137. In some embodiments, the antibody or antigen binding fragment thereof comprises a VH of SEQ ID NO: 143 and a VL of SEQ ID NO: 138.

In some embodiments, the antibody or antigen binding fragment thereof comprises a VH of SEQ ID NO: 144 and a VL of SEQ ID NO: 114. In some embodiments, the antibody or antigen binding fragment thereof comprises a VH of SEQ ID NO: 144 and a VL of SEQ ID NO: 116. In some embodiments, the antibody or antigen binding fragment thereof comprises a VH of SEQ ID NO: 144 and a VL of SEQ ID NO: 117. In some embodiments, the antibody or antigen binding fragment thereof comprises a VH of SEQ ID NO: 144 and a VL of SEQ ID NO: 118. In some embodiments, the antibody or antigen binding fragment thereof comprises a VH of SEQ ID NO: 144 and a VL of SEQ ID NO: 119. In some embodiments, the antibody or antigen binding fragment thereof comprises a VH of SEQ ID NO: 144 and a VL of SEQ ID NO: 120. In some embodiments, the antibody or antigen binding fragment thereof comprises a VH of SEQ ID NO: 144 and a VL of SEQ ID NO: 121. In some embodiments, the antibody or antigen binding fragment thereof comprises a VH of SEQ ID NO: 144 and a VL of SEQ ID NO: 122. In some embodiments, the antibody or antigen binding fragment thereof comprises a VH of SEQ ID NO: 144 and a VL of SEQ ID NO: 123. In some embodiments, the antibody or antigen binding fragment thereof comprises a VH of SEQ ID NO: 144 and a VL of SEQ ID NO: 124. In some embodiments, the antibody or antigen binding fragment thereof comprises a VH of SEQ ID NO: 144 and a VL of SEQ ID NO: 125. In some embodiments, the antibody or antigen binding fragment thereof comprises a VH of SEQ ID NO: 144 and a VL of SEQ ID NO: 126. In some embodiments, the antibody or antigen binding fragment thereof comprises a VH of SEQ ID NO: 144 and a VL of SEQ ID NO: 127. In some embodiments, the antibody or antigen binding fragment thereof comprises a VH of SEQ ID NO: 144 and a VL of SEQ ID NO: 128. In some embodiments, the antibody or antigen binding fragment thereof comprises a VH of SEQ ID NO: 144 and a VL of SEQ ID NO: 129. In some embodiments, the antibody or antigen binding fragment thereof comprises a VH of SEQ ID NO: 144 and a VL of SEQ ID NO: 130. In some embodiments, the antibody or antigen binding fragment thereof comprises a VH of SEQ ID NO: 144 and a VL of SEQ ID NO: 131. In some embodiments, the antibody or antigen binding fragment thereof comprises a VH of SEQ ID NO: 144 and a VL of SEQ ID NO: 132. In some embodiments, the antibody or antigen binding fragment thereof comprises a VH of SEQ ID NO: 144 and a VL of SEQ ID NO: 133. In some embodiments, the antibody or antigen binding fragment thereof comprises a VH of SEQ ID NO: 144 and a VL of SEQ ID NO: 134. In some embodiments, the antibody or antigen binding fragment thereof comprises a VH of SEQ ID NO: 144 and a VL of SEQ ID NO: 135. In some embodiments, the antibody or antigen binding fragment thereof comprises a VH of SEQ ID NO: 144 and a VL of SEQ ID NO: 136. In some embodiments, the antibody or antigen binding fragment thereof comprises a VH of SEQ ID NO: 144 and a VL of SEQ ID NO: 137. In some embodiments, the antibody or antigen binding fragment thereof comprises a VH of SEQ ID NO: 144 and a VL of SEQ ID NO: 138.

In some embodiments, the antibody or antigen binding fragment thereof comprises a VH of SEQ ID NO: 145 and a VL of SEQ ID NO: 114. In some embodiments, the antibody or antigen binding fragment thereof comprises a VH of SEQ ID NO: 145 and a VL of SEQ ID NO: 116. In some embodiments, the antibody or antigen binding fragment thereof comprises a VH of SEQ ID NO: 145 and a VL of SEQ ID NO: 117. In some embodiments, the antibody or antigen binding fragment thereof comprises a VH of SEQ ID NO: 145 and a VL of SEQ ID NO: 118. In some embodiments, the antibody or antigen binding fragment thereof comprises a VH of SEQ ID NO: 145 and a VL of SEQ ID NO: 119. In some embodiments, the antibody or antigen binding fragment thereof comprises a VH of SEQ ID NO: 145 and a VL of SEQ ID NO: 120. In some embodiments, the antibody or antigen binding fragment thereof comprises a VH of SEQ ID NO: 145 and a VL of SEQ ID NO: 121. In some embodiments, the antibody or antigen binding fragment thereof comprises a VH of SEQ ID NO: 145 and a VL of SEQ ID NO: 122. In some embodiments, the antibody or antigen binding fragment thereof comprises a VH of SEQ ID NO: 145 and a VL of SEQ ID NO: 123. In some embodiments, the antibody or antigen binding fragment thereof comprises a VH of SEQ ID NO: 145 and a VL of SEQ ID NO: 124. In some embodiments, the antibody or antigen binding fragment thereof comprises a VH of SEQ ID NO: 145 and a VL of SEQ ID NO: 125. In some embodiments, the antibody or antigen binding fragment thereof comprises a VH of SEQ ID NO: 145 and a VL of SEQ ID NO: 126. In some embodiments, the antibody or antigen binding fragment thereof comprises a VH of SEQ ID NO: 145 and a VL of SEQ ID NO: 127. In some embodiments, the antibody or antigen binding fragment thereof comprises a VH of SEQ ID NO: 145 and a VL of SEQ ID NO: 128. In some embodiments, the antibody or antigen binding fragment thereof comprises a VH of SEQ ID NO: 145 and a VL of SEQ ID NO: 129. In some embodiments, the antibody or antigen binding fragment thereof comprises a VH of SEQ ID NO: 145 and a VL of SEQ ID NO: 130. In some embodiments, the antibody or antigen binding fragment thereof comprises a VH of SEQ ID NO: 145 and a VL of SEQ ID NO: 131. In some embodiments, the antibody or antigen binding fragment thereof comprises a VH of SEQ ID NO: 145 and a VL of SEQ ID NO: 132. In some embodiments, the antibody or antigen binding fragment thereof comprises a VH of SEQ ID NO: 145 and a VL of SEQ ID NO: 133. In some embodiments, the antibody or antigen binding fragment thereof comprises a VH of SEQ ID NO: 145 and a VL of SEQ ID NO: 134. In some embodiments, the antibody or antigen binding fragment thereof comprises a VH of SEQ ID NO: 145 and a VL of SEQ ID NO: 135. In some embodiments, the antibody or antigen binding fragment thereof comprises a VH of SEQ ID NO: 145 and a VL of SEQ ID NO: 136. In some embodiments, the antibody or antigen binding fragment thereof comprises a VH of SEQ ID NO: 145 and a VL of SEQ ID NO: 137. In some embodiments, the antibody or antigen binding fragment thereof comprises a VH of SEQ ID NO: 145 and a VL of SEQ ID NO: 138.

In some embodiments, the antibody or antigen binding fragment thereof comprises a VH of SEQ ID NO: 146 and a VL of SEQ ID NO: 114. In some embodiments, the antibody or antigen binding fragment thereof comprises a VH of SEQ ID NO: 146 and a VL of SEQ ID NO: 116. In some embodiments, the antibody or antigen binding fragment thereof comprises a VH of SEQ ID NO: 146 and a VL of SEQ ID NO: 117. In some embodiments, the antibody or antigen binding fragment thereof comprises a VH of SEQ ID NO: 146 and a VL of SEQ ID NO: 118. In some embodiments, the antibody or antigen binding fragment thereof comprises a VH of SEQ ID NO: 146 and a VL of SEQ ID NO: 119. In some embodiments, the antibody or antigen binding fragment thereof comprises a VH of SEQ ID NO: 146 and a VL of SEQ ID NO: 120. In some embodiments, the antibody or antigen binding fragment thereof comprises a VH of SEQ ID NO: 146 and a VL of SEQ ID NO: 121. In some embodiments, the antibody or antigen binding fragment thereof comprises a VH of SEQ ID NO: 146 and a VL of SEQ ID NO: 122. In some embodiments, the antibody or antigen binding fragment thereof comprises a VH of SEQ ID NO: 146 and a VL of SEQ ID NO: 123. In some embodiments, the antibody or antigen binding fragment thereof comprises a VH of SEQ ID NO: 146 and a VL of SEQ ID NO: 124. In some embodiments, the antibody or antigen binding fragment thereof comprises a VH of SEQ ID NO: 146 and a VL of SEQ ID NO: 125. In some embodiments, the antibody or antigen binding fragment thereof comprises a VH of SEQ ID NO: 146 and a VL of SEQ ID NO: 126. In some embodiments, the antibody or antigen binding fragment thereof comprises a VH of SEQ ID NO: 146 and a VL of SEQ ID NO: 127. In some embodiments, the antibody or antigen binding fragment thereof comprises a VH of SEQ ID NO: 146 and a VL of SEQ ID NO: 128. In some embodiments, the antibody or antigen binding fragment thereof comprises a VH of SEQ ID NO: 146 and a VL of SEQ ID NO: 129. In some embodiments, the antibody or antigen binding fragment thereof comprises a VH of SEQ ID NO: 146 and a VL of SEQ ID NO: 130. In some embodiments, the antibody or antigen binding fragment thereof comprises a VH of SEQ ID NO: 146 and a VL of SEQ ID NO: 131. In some embodiments, the antibody or antigen binding fragment thereof comprises a VH of SEQ ID NO: 146 and a VL of SEQ ID NO: 132. In some embodiments, the antibody or antigen binding fragment thereof comprises a VH of SEQ ID NO: 146 and a VL of SEQ ID NO: 133. In some embodiments, the antibody or antigen binding fragment thereof comprises a VH of SEQ ID NO: 146 and a VL of SEQ ID NO: 134. In some embodiments, the antibody or antigen binding fragment thereof comprises a VH of SEQ ID NO: 146 and a VL of SEQ ID NO: 135. In some embodiments, the antibody or antigen binding fragment thereof comprises a VH of SEQ ID NO: 146 and a VL of SEQ ID NO: 136. In some embodiments, the antibody or antigen binding fragment thereof comprises a VH of SEQ ID NO: 146 and a VL of SEQ ID NO: 137. In some embodiments, the antibody or antigen binding fragment thereof comprises a VH of SEQ ID NO: 146 and a VL of SEQ ID NO: 138.

In some embodiments, the antibody or antigen binding fragment thereof comprises a VH of SEQ ID NO: 147 and a VL of SEQ ID NO: 114. In some embodiments, the antibody or antigen binding fragment thereof comprises a VH of SEQ ID NO: 147 and a VL of SEQ ID NO: 116. In some embodiments, the antibody or antigen binding fragment thereof comprises a VH of SEQ ID NO: 147 and a VL of SEQ ID NO: 117. In some embodiments, the antibody or antigen binding fragment thereof comprises a VH of SEQ ID NO: 147 and a VL of SEQ ID NO: 118. In some embodiments, the antibody or antigen binding fragment thereof comprises a VH of SEQ ID NO: 147 and a VL of SEQ ID NO: 119. In some embodiments, the antibody or antigen binding fragment thereof comprises a VH of SEQ ID NO: 147 and a VL of SEQ ID NO: 120. In some embodiments, the antibody or antigen binding fragment thereof comprises a VH of SEQ ID NO: 147 and a VL of SEQ ID NO: 121. In some embodiments, the antibody or antigen binding fragment thereof comprises a VH of SEQ ID NO: 147 and a VL of SEQ ID NO: 122. In some embodiments, the antibody or antigen binding fragment thereof comprises a VH of SEQ ID NO: 147 and a VL of SEQ ID NO: 123. In some embodiments, the antibody or antigen binding fragment thereof comprises a VH of SEQ ID NO: 147 and a VL of SEQ ID NO: 124. In some embodiments, the antibody or antigen binding fragment thereof comprises a VH of SEQ ID NO: 147 and a VL of SEQ ID NO: 125. In some embodiments, the antibody or antigen binding fragment thereof comprises a VH of SEQ ID NO: 147 and a VL of SEQ ID NO: 126. In some embodiments, the antibody or antigen binding fragment thereof comprises a VH of SEQ ID NO: 147 and a VL of SEQ ID NO: 127. In some embodiments, the antibody or antigen binding fragment thereof comprises a VH of SEQ ID NO: 147 and a VL of SEQ ID NO: 128. In some embodiments, the antibody or antigen binding fragment thereof comprises a VH of SEQ ID NO: 147 and a VL of SEQ ID NO: 129. In some embodiments, the antibody or antigen binding fragment thereof comprises a VH of SEQ ID NO: 147 and a VL of SEQ ID NO: 130. In some embodiments, the antibody or antigen binding fragment thereof comprises a VH of SEQ ID NO: 147 and a VL of SEQ ID NO: 131. In some embodiments, the antibody or antigen binding fragment thereof comprises a VH of SEQ ID NO: 147 and a VL of SEQ ID NO: 132. In some embodiments, the antibody or antigen binding fragment thereof comprises a VH of SEQ ID NO: 147 and a VL of SEQ ID NO: 133. In some embodiments, the antibody or antigen binding fragment thereof comprises a VH of SEQ ID NO: 147 and a VL of SEQ ID NO: 134. In some embodiments, the antibody or antigen binding fragment thereof comprises a VH of SEQ ID NO: 147 and a VL of SEQ ID NO: 135. In some embodiments, the antibody or antigen binding fragment thereof comprises a VH of SEQ ID NO: 147 and a VL of SEQ ID NO: 136. In some embodiments, the antibody or antigen binding fragment thereof comprises a VH of SEQ ID NO: 147 and a VL of SEQ ID NO: 137. In some embodiments, the antibody or antigen binding fragment thereof comprises a VH of SEQ ID NO: 147 and a VL of SEQ ID NO: 138.

In some embodiments, the antibody or antigen binding fragment thereof comprises a VH of SEQ ID NO: 148 and a VL of SEQ ID NO: 114. In some embodiments, the antibody or antigen binding fragment thereof comprises a VH of SEQ ID NO: 148 and a VL of SEQ ID NO: 116. In some embodiments, the antibody or antigen binding fragment thereof comprises a VH of SEQ ID NO: 148 and a VL of SEQ ID NO: 117. In some embodiments, the antibody or antigen binding fragment thereof comprises a VH of SEQ ID NO: 148 and a VL of SEQ ID NO: 118. In some embodiments, the antibody or antigen binding fragment thereof comprises a VH of SEQ ID NO: 148 and a VL of SEQ ID NO: 119. In some embodiments, the antibody or antigen binding fragment thereof comprises a VH of SEQ ID NO: 148 and a VL of SEQ ID NO: 120. In some embodiments, the antibody or antigen binding fragment thereof comprises a VH of SEQ ID NO: 148 and a VL of SEQ ID NO: 121. In some embodiments, the antibody or antigen binding fragment thereof comprises a VH of SEQ ID NO: 148 and a VL of SEQ ID NO: 122. In some embodiments, the antibody or antigen binding fragment thereof comprises a VH of SEQ ID NO: 148 and a VL of SEQ ID NO: 123. In some embodiments, the antibody or antigen binding fragment thereof comprises a VH of SEQ ID NO: 148 and a VL of SEQ ID NO: 124. In some embodiments, the antibody or antigen binding fragment thereof comprises a VH of SEQ ID NO: 148 and a VL of SEQ ID NO: 125. In some embodiments, the antibody or antigen binding fragment thereof comprises a VH of SEQ ID NO: 148 and a VL of SEQ ID NO: 126. In some embodiments, the antibody or antigen binding fragment thereof comprises a VH of SEQ ID NO: 148 and a VL of SEQ ID NO: 127. In some embodiments, the antibody or antigen binding fragment thereof comprises a VH of SEQ ID NO: 148 and a VL of SEQ ID NO: 128. In some embodiments, the antibody or antigen binding fragment thereof comprises a VH of SEQ ID NO: 148 and a VL of SEQ ID NO: 129. In some embodiments, the antibody or antigen binding fragment thereof comprises a VH of SEQ ID NO: 148 and a VL of SEQ ID NO: 130. In some embodiments, the antibody or antigen binding fragment thereof comprises a VH of SEQ ID NO: 148 and a VL of SEQ ID NO: 131. In some embodiments, the antibody or antigen binding fragment thereof comprises a VH of SEQ ID NO: 148 and a VL of SEQ ID NO: 132. In some embodiments, the antibody or antigen binding fragment thereof comprises a VH of SEQ ID NO: 148 and a VL of SEQ ID NO: 133. In some embodiments, the antibody or antigen binding fragment thereof comprises a VH of SEQ ID NO: 148 and a VL of SEQ ID NO: 134. In some embodiments, the antibody or antigen binding fragment thereof comprises a VH of SEQ ID NO: 148 and a VL of SEQ ID NO: 135. In some embodiments, the antibody or antigen binding fragment thereof comprises a VH of SEQ ID NO: 148 and a VL of SEQ ID NO: 136. In some embodiments, the antibody or antigen binding fragment thereof comprises a VH of SEQ ID NO: 148 and a VL of SEQ ID NO: 137. In some embodiments, the antibody or antigen binding fragment thereof comprises a VH of SEQ ID NO: 148 and a VL of SEQ ID NO: 138.

In some embodiments, the antibody or antigen binding fragment thereof comprises a VH of SEQ ID NO: 149 and a VL of SEQ ID NO: 114. In some embodiments, the antibody or antigen binding fragment thereof comprises a VH of SEQ ID NO: 149 and a VL of SEQ ID NO: 116. In some embodiments, the antibody or antigen binding fragment thereof comprises a VH of SEQ ID NO: 149 and a VL of SEQ ID NO: 117. In some embodiments, the antibody or antigen binding fragment thereof comprises a VH of SEQ ID NO: 149 and a VL of SEQ ID NO: 118. In some embodiments, the antibody or antigen binding fragment thereof comprises a VH of SEQ ID NO: 149 and a VL of SEQ ID NO: 119. In some embodiments, the antibody or antigen binding fragment thereof comprises a VH of SEQ ID NO: 149 and a VL of SEQ ID NO: 120. In some embodiments, the antibody or antigen binding fragment thereof comprises a VH of SEQ ID NO: 149 and a VL of SEQ ID NO: 121. In some embodiments, the antibody or antigen binding fragment thereof comprises a VH of SEQ ID NO: 149 and a VL of SEQ ID NO: 122. In some embodiments, the antibody or antigen binding fragment thereof comprises a VH of SEQ ID NO: 149 and a VL of SEQ ID NO: 123. In some embodiments, the antibody or antigen binding fragment thereof comprises a VH of SEQ ID NO: 149 and a VL of SEQ ID NO: 124. In some embodiments, the antibody or antigen binding fragment thereof comprises a VH of SEQ ID NO: 149 and a VL of SEQ ID NO: 125. In some embodiments, the antibody or antigen binding fragment thereof comprises a VH of SEQ ID NO: 149 and a VL of SEQ ID NO: 126. In some embodiments, the antibody or antigen binding fragment thereof comprises a VH of SEQ ID NO: 149 and a VL of SEQ ID NO: 127. In some embodiments, the antibody or antigen binding fragment thereof comprises a VH of SEQ ID NO: 149 and a VL of SEQ ID NO: 128. In some embodiments, the antibody or antigen binding fragment thereof comprises a VH of SEQ ID NO: 149 and a VL of SEQ ID NO: 129. In some embodiments, the antibody or antigen binding fragment thereof comprises a VH of SEQ ID NO: 149 and a VL of SEQ ID NO: 130. In some embodiments, the antibody or antigen binding fragment thereof comprises a VH of SEQ ID NO: 149 and a VL of SEQ ID NO: 131. In some embodiments, the antibody or antigen binding fragment thereof comprises a VH of SEQ ID NO: 149 and a VL of SEQ ID NO: 132. In some embodiments, the antibody or antigen binding fragment thereof comprises a VH of SEQ ID NO: 149 and a VL of SEQ ID NO: 133. In some embodiments, the antibody or antigen binding fragment thereof comprises a VH of SEQ ID NO: 149 and a VL of SEQ ID NO: 134. In some embodiments, the antibody or antigen binding fragment thereof comprises a VH of SEQ ID NO: 149 and a VL of SEQ ID NO: 135. In some embodiments, the antibody or antigen binding fragment thereof comprises a VH of SEQ ID NO: 149 and a VL of SEQ ID NO: 136. In some embodiments, the antibody or antigen binding fragment thereof comprises a VH of SEQ ID NO: 149 and a VL of SEQ ID NO: 137. In some embodiments, the antibody or antigen binding fragment thereof comprises a VH of SEQ ID NO: 149 and a VL of SEQ ID NO: 138.

In some embodiments, the antibody or antigen binding fragment thereof comprises a VH of SEQ ID NO: 150 and a VL of SEQ ID NO: 114. In some embodiments, the antibody or antigen binding fragment thereof comprises a VH of SEQ ID NO: 150 and a VL of SEQ ID NO: 116. In some embodiments, the antibody or antigen binding fragment thereof comprises a VH of SEQ ID NO: 150 and a VL of SEQ ID NO: 117. In some embodiments, the antibody or antigen binding fragment thereof comprises a VH of SEQ ID NO: 150 and a VL of SEQ ID NO: 118. In some embodiments, the antibody or antigen binding fragment thereof comprises a VH of SEQ ID NO: 150 and a VL of SEQ ID NO: 119. In some embodiments, the antibody or antigen binding fragment thereof comprises a VH of SEQ ID NO: 150 and a VL of SEQ ID NO: 120. In some embodiments, the antibody or antigen binding fragment thereof comprises a VH of SEQ ID NO: 150 and a VL of SEQ ID NO: 121. In some embodiments, the antibody or antigen binding fragment thereof comprises a VH of SEQ ID NO: 150 and a VL of SEQ ID NO: 122. In some embodiments, the antibody or antigen binding fragment thereof comprises a VH of SEQ ID NO: 150 and a VL of SEQ ID NO: 123. In some embodiments, the antibody or antigen binding fragment thereof comprises a VH of SEQ ID NO: 150 and a VL of SEQ ID NO: 124. In some embodiments, the antibody or antigen binding fragment thereof comprises a VH of SEQ ID NO: 150 and a VL of SEQ ID NO: 125. In some embodiments, the antibody or antigen binding fragment thereof comprises a VH of SEQ ID NO: 150 and a VL of SEQ ID NO: 126. In some embodiments, the antibody or antigen binding fragment thereof comprises a VH of SEQ ID NO: 150 and a VL of SEQ ID NO: 127. In some embodiments, the antibody or antigen binding fragment thereof comprises a VH of SEQ ID NO: 150 and a VL of SEQ ID NO: 128. In some embodiments, the antibody or antigen binding fragment thereof comprises a VH of SEQ ID NO: 150 and a VL of SEQ ID NO: 129. In some embodiments, the antibody or antigen binding fragment thereof comprises a VH of SEQ ID NO: 150 and a VL of SEQ ID NO: 130. In some embodiments, the antibody or antigen binding fragment thereof comprises a VH of SEQ ID NO: 150 and a VL of SEQ ID NO: 131. In some embodiments, the antibody or antigen binding fragment thereof comprises a VH of SEQ ID NO: 150 and a VL of SEQ ID NO: 132. In some embodiments, the antibody or antigen binding fragment thereof comprises a VH of SEQ ID NO: 150 and a VL of SEQ ID NO: 133. In some embodiments, the antibody or antigen binding fragment thereof comprises a VH of SEQ ID NO: 150 and a VL of SEQ ID NO: 134. In some embodiments, the antibody or antigen binding fragment thereof comprises a VH of SEQ ID NO: 150 and a VL of SEQ ID NO: 135. In some embodiments, the antibody or antigen binding fragment thereof comprises a VH of SEQ ID NO: 150 and a VL of SEQ ID NO: 136. In some embodiments, the antibody or antigen binding fragment thereof comprises a VH of SEQ ID NO: 150 and a VL of SEQ ID NO: 137. In some embodiments, the antibody or antigen binding fragment thereof comprises a VH of SEQ ID NO: 150 and a VL of SEQ ID NO: 138.

In some embodiments, the antibody or antigen binding fragment thereof comprises a VH of SEQ ID NO: 151 and a VL of SEQ ID NO: 114. In some embodiments, the antibody or antigen binding fragment thereof comprises a VH of SEQ ID NO: 151 and a VL of SEQ ID NO: 116. In some embodiments, the antibody or antigen binding fragment thereof comprises a VH of SEQ ID NO: 151 and a VL of SEQ ID NO: 117. In some embodiments, the antibody or antigen binding fragment thereof comprises a VH of SEQ ID NO: 151 and a VL of SEQ ID NO: 118. In some embodiments, the antibody or antigen binding fragment thereof comprises a VH of SEQ ID NO: 151 and a VL of SEQ ID NO: 119. In some embodiments, the antibody or antigen binding fragment thereof comprises a VH of SEQ ID NO: 151 and a VL of SEQ ID NO: 120. In some embodiments, the antibody or antigen binding fragment thereof comprises a VH of SEQ ID NO: 151 and a VL of SEQ ID NO: 121. In some embodiments, the antibody or antigen binding fragment thereof comprises a VH of SEQ ID NO: 151 and a VL of SEQ ID NO: 122. In some embodiments, the antibody or antigen binding fragment thereof comprises a VH of SEQ ID NO: 151 and a VL of SEQ ID NO: 123. In some embodiments, the antibody or antigen binding fragment thereof comprises a VH of SEQ ID NO: 151 and a VL of SEQ ID NO: 124. In some embodiments, the antibody or antigen binding fragment thereof comprises a VH of SEQ ID NO: 151 and a VL of SEQ ID NO: 125. In some embodiments, the antibody or antigen binding fragment thereof comprises a VH of SEQ ID NO: 151 and a VL of SEQ ID NO: 126. In some embodiments, the antibody or antigen binding fragment thereof comprises a VH of SEQ ID NO: 151 and a VL of SEQ ID NO: 127. In some embodiments, the antibody or antigen binding fragment thereof comprises a VH of SEQ ID NO: 151 and a VL of SEQ ID NO: 128. In some embodiments, the antibody or antigen binding fragment thereof comprises a VH of SEQ ID NO: 151 and a VL of SEQ ID NO: 129. In some embodiments, the antibody or antigen binding fragment thereof comprises a VH of SEQ ID NO: 151 and a VL of SEQ ID NO: 130. In some embodiments, the antibody or antigen binding fragment thereof comprises a VH of SEQ ID NO: 151 and a VL of SEQ ID NO: 131. In some embodiments, the antibody or antigen binding fragment thereof comprises a VH of SEQ ID NO: 151 and a VL of SEQ ID NO: 132. In some embodiments, the antibody or antigen binding fragment thereof comprises a VH of SEQ ID NO: 151 and a VL of SEQ ID NO: 133. In some embodiments, the antibody or antigen binding fragment thereof comprises a VH of SEQ ID NO: 151 and a VL of SEQ ID NO: 134. In some embodiments, the antibody or antigen binding fragment thereof comprises a VH of SEQ ID NO: 151 and a VL of SEQ ID NO: 135. In some embodiments, the antibody or antigen binding fragment thereof comprises a VH of SEQ ID NO: 151 and a VL of SEQ ID NO: 136. In some embodiments, the antibody or antigen binding fragment thereof comprises a VH of SEQ ID NO: 151 and a VL of SEQ ID NO: 137. In some embodiments, the antibody or antigen binding fragment thereof comprises a VH of SEQ ID NO: 151 and a VL of SEQ ID NO: 138.

In some embodiments, the antibody or antigen binding fragment thereof comprises a VH of SEQ ID NO: 152 and a VL of SEQ ID NO: 114. In some embodiments, the antibody or antigen binding fragment thereof comprises a VH of SEQ ID NO: 152 and a VL of SEQ ID NO: 116. In some embodiments, the antibody or antigen binding fragment thereof comprises a VH of SEQ ID NO: 152 and a VL of SEQ ID NO: 117. In some embodiments, the antibody or antigen binding fragment thereof comprises a VH of SEQ ID NO: 152 and a VL of SEQ ID NO: 118. In some embodiments, the antibody or antigen binding fragment thereof comprises a VH of SEQ ID NO: 152 and a VL of SEQ ID NO: 119. In some embodiments, the antibody or antigen binding fragment thereof comprises a VH of SEQ ID NO: 152 and a VL of SEQ ID NO: 120. In some embodiments, the antibody or antigen binding fragment thereof comprises a VH of SEQ ID NO: 152 and a VL of SEQ ID NO: 121. In some embodiments, the antibody or antigen binding fragment thereof comprises a VH of SEQ ID NO: 152 and a VL of SEQ ID NO: 122. In some embodiments, the antibody or antigen binding fragment thereof comprises a VH of SEQ ID NO: 152 and a VL of SEQ ID NO: 123. In some embodiments, the antibody or antigen binding fragment thereof comprises a VH of SEQ ID NO: 152 and a VL of SEQ ID NO: 124. In some embodiments, the antibody or antigen binding fragment thereof comprises a VH of SEQ ID NO: 152 and a VL of SEQ ID NO: 125. In some embodiments, the antibody or antigen binding fragment thereof comprises a VH of SEQ ID NO: 152 and a VL of SEQ ID NO: 126. In some embodiments, the antibody or antigen binding fragment thereof comprises a VH of SEQ ID NO: 152 and a VL of SEQ ID NO: 127. In some embodiments, the antibody or antigen binding fragment thereof comprises a VH of SEQ ID NO: 152 and a VL of SEQ ID NO: 128. In some embodiments, the antibody or antigen binding fragment thereof comprises a VH of SEQ ID NO: 152 and a VL of SEQ ID NO: 129. In some embodiments, the antibody or antigen binding fragment thereof comprises a VH of SEQ ID NO: 152 and a VL of SEQ ID NO: 130. In some embodiments, the antibody or antigen binding fragment thereof comprises a VH of SEQ ID NO: 152 and a VL of SEQ ID NO: 131. In some embodiments, the antibody or antigen binding fragment thereof comprises a VH of SEQ ID NO: 152 and a VL of SEQ ID NO: 132. In some embodiments, the antibody or antigen binding fragment thereof comprises a VH of SEQ ID NO: 152 and a VL of SEQ ID NO: 133. In some embodiments, the antibody or antigen binding fragment thereof comprises a VH of SEQ ID NO: 152 and a VL of SEQ ID NO: 134. In some embodiments, the antibody or antigen binding fragment thereof comprises a VH of SEQ ID NO: 152 and a VL of SEQ ID NO: 135. In some embodiments, the antibody or antigen binding fragment thereof comprises a VH of SEQ ID NO: 152 and a VL of SEQ ID NO: 136. In some embodiments, the antibody or antigen binding fragment thereof comprises a VH of SEQ ID NO: 152 and a VL of SEQ ID NO: 137. In some embodiments, the antibody or antigen binding fragment thereof comprises a VH of SEQ ID NO: 152 and a VL of SEQ ID NO: 138.

In some embodiments, the antibody or antigen binding fragment thereof comprises a VH of SEQ ID NO: 153 and a VL of SEQ ID NO: 114. In some embodiments, the antibody or antigen binding fragment thereof comprises a VH of SEQ ID NO: 153 and a VL of SEQ ID NO: 116. In some embodiments, the antibody or antigen binding fragment thereof comprises a VH of SEQ ID NO: 153 and a VL of SEQ ID NO: 117. In some embodiments, the antibody or antigen binding fragment thereof comprises a VH of SEQ ID NO: 153 and a VL of SEQ ID NO: 118. In some embodiments, the antibody or antigen binding fragment thereof comprises a VH of SEQ ID NO: 153 and a VL of SEQ ID NO: 119. In some embodiments, the antibody or antigen binding fragment thereof comprises a VH of SEQ ID NO: 153 and a VL of SEQ ID NO: 120. In some embodiments, the antibody or antigen binding fragment thereof comprises a VH of SEQ ID NO: 153 and a VL of SEQ ID NO: 121. In some embodiments, the antibody or antigen binding fragment thereof comprises a VH of SEQ ID NO: 153 and a VL of SEQ ID NO: 122. In some embodiments, the antibody or antigen binding fragment thereof comprises a VH of SEQ ID NO: 153 and a VL of SEQ ID NO: 123. In some embodiments, the antibody or antigen binding fragment thereof comprises a VH of SEQ ID NO: 153 and a VL of SEQ ID NO: 124. In some embodiments, the antibody or antigen binding fragment thereof comprises a VH of SEQ ID NO: 153 and a VL of SEQ ID NO: 125. In some embodiments, the antibody or antigen binding fragment thereof comprises a VH of SEQ ID NO: 153 and a VL of SEQ ID NO: 126. In some embodiments, the antibody or antigen binding fragment thereof comprises a VH of SEQ ID NO: 153 and a VL of SEQ ID NO: 127. In some embodiments, the antibody or antigen binding fragment thereof comprises a VH of SEQ ID NO: 153 and a VL of SEQ ID NO: 128. In some embodiments, the antibody or antigen binding fragment thereof comprises a VH of SEQ ID NO: 153 and a VL of SEQ ID NO: 129. In some embodiments, the antibody or antigen binding fragment thereof comprises a VH of SEQ ID NO: 153 and a VL of SEQ ID NO: 130. In some embodiments, the antibody or antigen binding fragment thereof comprises a VH of SEQ ID NO: 153 and a VL of SEQ ID NO: 131. In some embodiments, the antibody or antigen binding fragment thereof comprises a VH of SEQ ID NO: 153 and a VL of SEQ ID NO: 132. In some embodiments, the antibody or antigen binding fragment thereof comprises a VH of SEQ ID NO: 153 and a VL of SEQ ID NO: 133. In some embodiments, the antibody or antigen binding fragment thereof comprises a VH of SEQ ID NO: 153 and a VL of SEQ ID NO: 134. In some embodiments, the antibody or antigen binding fragment thereof comprises a VH of SEQ ID NO: 153 and a VL of SEQ ID NO: 135. In some embodiments, the antibody or antigen binding fragment thereof comprises a VH of SEQ ID NO: 153 and a VL of SEQ ID NO: 136. In some embodiments, the antibody or antigen binding fragment thereof comprises a VH of SEQ ID NO: 153 and a VL of SEQ ID NO: 137. In some embodiments, the antibody or antigen binding fragment thereof comprises a VH of SEQ ID NO: 153 and a VL of SEQ ID NO: 138.

In some embodiments, the antibody or antigen binding fragment thereof comprises a VH of SEQ ID NO: 154 and a VL of SEQ ID NO: 114. In some embodiments, the antibody or antigen binding fragment thereof comprises a VH of SEQ ID NO: 154 and a VL of SEQ ID NO: 116. In some embodiments, the antibody or antigen binding fragment thereof comprises a VH of SEQ ID NO: 154 and a VL of SEQ ID NO: 117. In some embodiments, the antibody or antigen binding fragment thereof comprises a VH of SEQ ID NO: 154 and a VL of SEQ ID NO: 118. In some embodiments, the antibody or antigen binding fragment thereof comprises a VH of SEQ ID NO: 154 and a VL of SEQ ID NO: 119. In some embodiments, the antibody or antigen binding fragment thereof comprises a VH of SEQ ID NO: 154 and a VL of SEQ ID NO: 120. In some embodiments, the antibody or antigen binding fragment thereof comprises a VH of SEQ ID NO: 154 and a VL of SEQ ID NO: 121. In some embodiments, the antibody or antigen binding fragment thereof comprises a VH of SEQ ID NO: 154 and a VL of SEQ ID NO: 122. In some embodiments, the antibody or antigen binding fragment thereof comprises a VH of SEQ ID NO: 154 and a VL of SEQ ID NO: 123. In some embodiments, the antibody or antigen binding fragment thereof comprises a VH of SEQ ID NO: 154 and a VL of SEQ ID NO: 124. In some embodiments, the antibody or antigen binding fragment thereof comprises a VH of SEQ ID NO: 154 and a VL of SEQ ID NO: 125. In some embodiments, the antibody or antigen binding fragment thereof comprises a VH of SEQ ID NO: 154 and a VL of SEQ ID NO: 126. In some embodiments, the antibody or antigen binding fragment thereof comprises a VH of SEQ ID NO: 154 and a VL of SEQ ID NO: 127. In some embodiments, the antibody or antigen binding fragment thereof comprises a VH of SEQ ID NO: 154 and a VL of SEQ ID NO: 128. In some embodiments, the antibody or antigen binding fragment thereof comprises a VH of SEQ ID NO: 154 and a VL of SEQ ID NO: 129. In some embodiments, the antibody or antigen binding fragment thereof comprises a VH of SEQ ID NO: 154 and a VL of SEQ ID NO: 130. In some embodiments, the antibody or antigen binding fragment thereof comprises a VH of SEQ ID NO: 154 and a VL of SEQ ID NO: 131. In some embodiments, the antibody or antigen binding fragment thereof comprises a VH of SEQ ID NO: 154 and a VL of SEQ ID NO: 132. In some embodiments, the antibody or antigen binding fragment thereof comprises a VH of SEQ ID NO: 154 and a VL of SEQ ID NO: 133. In some embodiments, the antibody or antigen binding fragment thereof comprises a VH of SEQ ID NO: 154 and a VL of SEQ ID NO: 134. In some embodiments, the antibody or antigen binding fragment thereof comprises a VH of SEQ ID NO: 154 and a VL of SEQ ID NO: 135. In some embodiments, the antibody or antigen binding fragment thereof comprises a VH of SEQ ID NO: 154 and a VL of SEQ ID NO: 136. In some embodiments, the antibody or antigen binding fragment thereof comprises a VH of SEQ ID NO: 154 and a VL of SEQ ID NO: 137. In some embodiments, the antibody or antigen binding fragment thereof comprises a VH of SEQ ID NO: 154 and a VL of SEQ ID NO: 138.

In some embodiments, the antibody or antigen binding fragment thereof comprises a VH of SEQ ID NO: 155 and a VL of SEQ ID NO: 114. In some embodiments, the antibody or antigen binding fragment thereof comprises a VH of SEQ ID NO: 155 and a VL of SEQ ID NO: 116. In some embodiments, the antibody or antigen binding fragment thereof comprises a VH of SEQ ID NO: 155 and a VL of SEQ ID NO: 117. In some embodiments, the antibody or antigen binding fragment thereof comprises a VH of SEQ ID NO: 155 and a VL of SEQ ID NO: 118. In some embodiments, the antibody or antigen binding fragment thereof comprises a VH of SEQ ID NO: 155 and a VL of SEQ ID NO: 119. In some embodiments, the antibody or antigen binding fragment thereof comprises a VH of SEQ ID NO: 155 and a VL of SEQ ID NO: 120. In some embodiments, the antibody or antigen binding fragment thereof comprises a VH of SEQ ID NO: 155 and a VL of SEQ ID NO: 121. In some embodiments, the antibody or antigen binding fragment thereof comprises a VH of SEQ ID NO: 155 and a VL of SEQ ID NO: 122. In some embodiments, the antibody or antigen binding fragment thereof comprises a VH of SEQ ID NO: 155 and a VL of SEQ ID NO: 123. In some embodiments, the antibody or antigen binding fragment thereof comprises a VH of SEQ ID NO: 155 and a VL of SEQ ID NO: 124. In some embodiments, the antibody or antigen binding fragment thereof comprises a VH of SEQ ID NO: 155 and a VL of SEQ ID NO: 125. In some embodiments, the antibody or antigen binding fragment thereof comprises a VH of SEQ ID NO: 155 and a VL of SEQ ID NO: 126. In some embodiments, the antibody or antigen binding fragment thereof comprises a VH of SEQ ID NO: 155 and a VL of SEQ ID NO: 127. In some embodiments, the antibody or antigen binding fragment thereof comprises a VH of SEQ ID NO: 155 and a VL of SEQ ID NO: 128. In some embodiments, the antibody or antigen binding fragment thereof comprises a VH of SEQ ID NO: 155 and a VL of SEQ ID NO: 129. In some embodiments, the antibody or antigen binding fragment thereof comprises a VH of SEQ ID NO: 155 and a VL of SEQ ID NO: 130. In some embodiments, the antibody or antigen binding fragment thereof comprises a VH of SEQ ID NO: 155 and a VL of SEQ ID NO: 131. In some embodiments, the antibody or antigen binding fragment thereof comprises a VH of SEQ ID NO: 155 and a VL of SEQ ID NO: 132. In some embodiments, the antibody or antigen binding fragment thereof comprises a VH of SEQ ID NO: 155 and a VL of SEQ ID NO: 133. In some embodiments, the antibody or antigen binding fragment thereof comprises a VH of SEQ ID NO: 155 and a VL of SEQ ID NO: 134. In some embodiments, the antibody or antigen binding fragment thereof comprises a VH of SEQ ID NO: 155 and a VL of SEQ ID NO: 135. In some embodiments, the antibody or antigen binding fragment thereof comprises a VH of SEQ ID NO: 155 and a VL of SEQ ID NO: 136. In some embodiments, the antibody or antigen binding fragment thereof comprises a VH of SEQ ID NO: 155 and a VL of SEQ ID NO: 137. In some embodiments, the antibody or antigen binding fragment thereof comprises a VH of SEQ ID NO: 155 and a VL of SEQ ID NO: 138.

In some embodiments, the antibody or antigen binding fragment thereof comprises a VH of SEQ ID NO: 156 and a VL of SEQ ID NO: 114. In some embodiments, the antibody or antigen binding fragment thereof comprises a VH of SEQ ID NO: 156 and a VL of SEQ ID NO: 116. In some embodiments, the antibody or antigen binding fragment thereof comprises a VH of SEQ ID NO: 156 and a VL of SEQ ID NO: 117. In some embodiments, the antibody or antigen binding fragment thereof comprises a VH of SEQ ID NO: 156 and a VL of SEQ ID NO: 118. In some embodiments, the antibody or antigen binding fragment thereof comprises a VH of SEQ ID NO: 156 and a VL of SEQ ID NO: 119. In some embodiments, the antibody or antigen binding fragment thereof comprises a VH of SEQ ID NO: 156 and a VL of SEQ ID NO: 120. In some embodiments, the antibody or antigen binding fragment thereof comprises a VH of SEQ ID NO: 156 and a VL of SEQ ID NO: 121. In some embodiments, the antibody or antigen binding fragment thereof comprises a VH of SEQ ID NO: 156 and a VL of SEQ ID NO: 122. In some embodiments, the antibody or antigen binding fragment thereof comprises a VH of SEQ ID NO: 156 and a VL of SEQ ID NO: 123. In some embodiments, the antibody or antigen binding fragment thereof comprises a VH of SEQ ID NO: 156 and a VL of SEQ ID NO: 124. In some embodiments, the antibody or antigen binding fragment thereof comprises a VH of SEQ ID NO: 156 and a VL of SEQ ID NO: 125. In some embodiments, the antibody or antigen binding fragment thereof comprises a VH of SEQ ID NO: 156 and a VL of SEQ ID NO: 126. In some embodiments, the antibody or antigen binding fragment thereof comprises a VH of SEQ ID NO: 156 and a VL of SEQ ID NO: 127. In some embodiments, the antibody or antigen binding fragment thereof comprises a VH of SEQ ID NO: 156 and a VL of SEQ ID NO: 128. In some embodiments, the antibody or antigen binding fragment thereof comprises a VH of SEQ ID NO: 156 and a VL of SEQ ID NO: 129. In some embodiments, the antibody or antigen binding fragment thereof comprises a VH of SEQ ID NO: 156 and a VL of SEQ ID NO: 130. In some embodiments, the antibody or antigen binding fragment thereof comprises a VH of SEQ ID NO: 156 and a VL of SEQ ID NO: 131. In some embodiments, the antibody or antigen binding fragment thereof comprises a VH of SEQ ID NO: 156 and a VL of SEQ ID NO: 132. In some embodiments, the antibody or antigen binding fragment thereof comprises a VH of SEQ ID NO: 156 and a VL of SEQ ID NO: 133. In some embodiments, the antibody or antigen binding fragment thereof comprises a VH of SEQ ID NO: 156 and a VL of SEQ ID NO: 134. In some embodiments, the antibody or antigen binding fragment thereof comprises a VH of SEQ ID NO: 156 and a VL of SEQ ID NO: 135. In some embodiments, the antibody or antigen binding fragment thereof comprises a VH of SEQ ID NO: 156 and a VL of SEQ ID NO: 136. In some embodiments, the antibody or antigen binding fragment thereof comprises a VH of SEQ ID NO: 156 and a VL of SEQ ID NO: 137. In some embodiments, the antibody or antigen binding fragment thereof comprises a VH of SEQ ID NO: 156 and a VL of SEQ ID NO: 138.

In some embodiments, the antibody or antigen binding fragment thereof comprises a VH of SEQ ID NO: 157 and a VL of SEQ ID NO: 114. In some embodiments, the antibody or antigen binding fragment thereof comprises a VH of SEQ ID NO: 157 and a VL of SEQ ID NO: 116. In some embodiments, the antibody or antigen binding fragment thereof comprises a VH of SEQ ID NO: 157 and a VL of SEQ ID NO: 117. In some embodiments, the antibody or antigen binding fragment thereof comprises a VH of SEQ ID NO: 157 and a VL of SEQ ID NO: 118. In some embodiments, the antibody or antigen binding fragment thereof comprises a VH of SEQ ID NO: 157 and a VL of SEQ ID NO: 119. In some embodiments, the antibody or antigen binding fragment thereof comprises a VH of SEQ ID NO: 157 and a VL of SEQ ID NO: 120. In some embodiments, the antibody or antigen binding fragment thereof comprises a VH of SEQ ID NO: 157 and a VL of SEQ ID NO: 121. In some embodiments, the antibody or antigen binding fragment thereof comprises a VH of SEQ ID NO: 157 and a VL of SEQ ID NO: 122. In some embodiments, the antibody or antigen binding fragment thereof comprises a VH of SEQ ID NO: 157 and a VL of SEQ ID NO: 123. In some embodiments, the antibody or antigen binding fragment thereof comprises a VH of SEQ ID NO: 157 and a VL of SEQ ID NO: 124. In some embodiments, the antibody or antigen binding fragment thereof comprises a VH of SEQ ID NO: 157 and a VL of SEQ ID NO: 125. In some embodiments, the antibody or antigen binding fragment thereof comprises a VH of SEQ ID NO: 157 and a VL of SEQ ID NO: 126. In some embodiments, the antibody or antigen binding fragment thereof comprises a VH of SEQ ID NO: 157 and a VL of SEQ ID NO: 127. In some embodiments, the antibody or antigen binding fragment thereof comprises a VH of SEQ ID NO: 157 and a VL of SEQ ID NO: 128. In some embodiments, the antibody or antigen binding fragment thereof comprises a VH of SEQ ID NO: 157 and a VL of SEQ ID NO: 129. In some embodiments, the antibody or antigen binding fragment thereof comprises a VH of SEQ ID NO: 157 and a VL of SEQ ID NO: 130. In some embodiments, the antibody or antigen binding fragment thereof comprises a VH of SEQ ID NO: 157 and a VL of SEQ ID NO: 131. In some embodiments, the antibody or antigen binding fragment thereof comprises a VH of SEQ ID NO: 157 and a VL of SEQ ID NO: 132. In some embodiments, the antibody or antigen binding fragment thereof comprises a VH of SEQ ID NO: 157 and a VL of SEQ ID NO: 133. In some embodiments, the antibody or antigen binding fragment thereof comprises a VH of SEQ ID NO: 157 and a VL of SEQ ID NO: 134. In some embodiments, the antibody or antigen binding fragment thereof comprises a VH of SEQ ID NO: 157 and a VL of SEQ ID NO: 135. In some embodiments, the antibody or antigen binding fragment thereof comprises a VH of SEQ ID NO: 157 and a VL of SEQ ID NO: 136. In some embodiments, the antibody or antigen binding fragment thereof comprises a VH of SEQ ID NO: 157 and a VL of SEQ ID NO: 137. In some embodiments, the antibody or antigen binding fragment thereof comprises a VH of SEQ ID NO: 157 and a VL of SEQ ID NO: 138.

In some embodiments, the antibody or antigen binding fragment thereof comprises a VH of SEQ ID NO: 158 and a VL of SEQ ID NO: 114. In some embodiments, the antibody or antigen binding fragment thereof comprises a VH of SEQ ID NO: 158 and a VL of SEQ ID NO: 116. In some embodiments, the antibody or antigen binding fragment thereof comprises a VH of SEQ ID NO: 158 and a VL of SEQ ID NO: 117. In some embodiments, the antibody or antigen binding fragment thereof comprises a VH of SEQ ID NO: 158 and a VL of SEQ ID NO: 118. In some embodiments, the antibody or antigen binding fragment thereof comprises a VH of SEQ ID NO: 158 and a VL of SEQ ID NO: 119. In some embodiments, the antibody or antigen binding fragment thereof comprises a VH of SEQ ID NO: 158 and a VL of SEQ ID NO: 120. In some embodiments, the antibody or antigen binding fragment thereof comprises a VH of SEQ ID NO: 158 and a VL of SEQ ID NO: 121. In some embodiments, the antibody or antigen binding fragment thereof comprises a VH of SEQ ID NO: 158 and a VL of SEQ ID NO: 122. In some embodiments, the antibody or antigen binding fragment thereof comprises a VH of SEQ ID NO: 158 and a VL of SEQ ID NO: 123. In some embodiments, the antibody or antigen binding fragment thereof comprises a VH of SEQ ID NO: 158 and a VL of SEQ ID NO: 124. In some embodiments, the antibody or antigen binding fragment thereof comprises a VH of SEQ ID NO: 158 and a VL of SEQ ID NO: 125. In some embodiments, the antibody or antigen binding fragment thereof comprises a VH of SEQ ID NO: 158 and a VL of SEQ ID NO: 126. In some embodiments, the antibody or antigen binding fragment thereof comprises a VH of SEQ ID NO: 158 and a VL of SEQ ID NO: 127. In some embodiments, the antibody or antigen binding fragment thereof comprises a VH of SEQ ID NO: 158 and a VL of SEQ ID NO: 128. In some embodiments, the antibody or antigen binding fragment thereof comprises a VH of SEQ ID NO: 158 and a VL of SEQ ID NO: 129. In some embodiments, the antibody or antigen binding fragment thereof comprises a VH of SEQ ID NO: 158 and a VL of SEQ ID NO: 130. In some embodiments, the antibody or antigen binding fragment thereof comprises a VH of SEQ ID NO: 158 and a VL of SEQ ID NO: 131. In some embodiments, the antibody or antigen binding fragment thereof comprises a VH of SEQ ID NO: 158 and a VL of SEQ ID NO: 132. In some embodiments, the antibody or antigen binding fragment thereof comprises a VH of SEQ ID NO: 158 and a VL of SEQ ID NO: 133. In some embodiments, the antibody or antigen binding fragment thereof comprises a VH of SEQ ID NO: 158 and a VL of SEQ ID NO: 134. In some embodiments, the antibody or antigen binding fragment thereof comprises a VH of SEQ ID NO: 158 and a VL of SEQ ID NO: 135. In some embodiments, the antibody or antigen binding fragment thereof comprises a VH of SEQ ID NO: 158 and a VL of SEQ ID NO: 136. In some embodiments, the antibody or antigen binding fragment thereof comprises a VH of SEQ ID NO: 158 and a VL of SEQ ID NO: 137. In some embodiments, the antibody or antigen binding fragment thereof comprises a VH of SEQ ID NO: 158 and a VL of SEQ ID NO: 138.

In some embodiments, the antibody or antigen binding fragment thereof comprises a VH of SEQ ID NO: 159 and a VL of SEQ ID NO: 114. In some embodiments, the antibody or antigen binding fragment thereof comprises a VH of SEQ ID NO: 159 and a VL of SEQ ID NO: 116. In some embodiments, the antibody or antigen binding fragment thereof comprises a VH of SEQ ID NO: 159 and a VL of SEQ ID NO: 117. In some embodiments, the antibody or antigen binding fragment thereof comprises a VH of SEQ ID NO: 159 and a VL of SEQ ID NO: 118. In some embodiments, the antibody or antigen binding fragment thereof comprises a VH of SEQ ID NO: 159 and a VL of SEQ ID NO: 119. In some embodiments, the antibody or antigen binding fragment thereof comprises a VH of SEQ ID NO: 159 and a VL of SEQ ID NO: 120. In some embodiments, the antibody or antigen binding fragment thereof comprises a VH of SEQ ID NO: 159 and a VL of SEQ ID NO: 121. In some embodiments, the antibody or antigen binding fragment thereof comprises a VH of SEQ ID NO: 159 and a VL of SEQ ID NO: 122. In some embodiments, the antibody or antigen binding fragment thereof comprises a VH of SEQ ID NO: 159 and a VL of SEQ ID NO: 123. In some embodiments, the antibody or antigen binding fragment thereof comprises a VH of SEQ ID NO: 159 and a VL of SEQ ID NO: 124. In some embodiments, the antibody or antigen binding fragment thereof comprises a VH of SEQ ID NO: 159 and a VL of SEQ ID NO: 125. In some embodiments, the antibody or antigen binding fragment thereof comprises a VH of SEQ ID NO: 159 and a VL of SEQ ID NO: 126. In some embodiments, the antibody or antigen binding fragment thereof comprises a VH of SEQ ID NO: 159 and a VL of SEQ ID NO: 127. In some embodiments, the antibody or antigen binding fragment thereof comprises a VH of SEQ ID NO: 159 and a VL of SEQ ID NO: 128. In some embodiments, the antibody or antigen binding fragment thereof comprises a VH of SEQ ID NO: 159 and a VL of SEQ ID NO: 129. In some embodiments, the antibody or antigen binding fragment thereof comprises a VH of SEQ ID NO: 159 and a VL of SEQ ID NO: 130. In some embodiments, the antibody or antigen binding fragment thereof comprises a VH of SEQ ID NO: 159 and a VL of SEQ ID NO: 131. In some embodiments, the antibody or antigen binding fragment thereof comprises a VH of SEQ ID NO: 159 and a VL of SEQ ID NO: 132. In some embodiments, the antibody or antigen binding fragment thereof comprises a VH of SEQ ID NO: 159 and a VL of SEQ ID NO: 133. In some embodiments, the antibody or antigen binding fragment thereof comprises a VH of SEQ ID NO: 159 and a VL of SEQ ID NO: 134. In some embodiments, the antibody or antigen binding fragment thereof comprises a VH of SEQ ID NO: 159 and a VL of SEQ ID NO: 135. In some embodiments, the antibody or antigen binding fragment thereof comprises a VH of SEQ ID NO: 159 and a VL of SEQ ID NO: 136. In some embodiments, the antibody or antigen binding fragment thereof comprises a VH of SEQ ID NO: 159 and a VL of SEQ ID NO: 137. In some embodiments, the antibody or antigen binding fragment thereof comprises a VH of SEQ ID NO: 159 and a VL of SEQ ID NO: 138.

In some embodiments, the antibody or antigen binding fragment thereof comprises a VH of SEQ ID NO: 160 and a VL of SEQ ID NO: 114. In some embodiments, the antibody or antigen binding fragment thereof comprises a VH of SEQ ID NO: 160 and a VL of SEQ ID NO: 116. In some embodiments, the antibody or antigen binding fragment thereof comprises a VH of SEQ ID NO: 160 and a VL of SEQ ID NO: 117. In some embodiments, the antibody or antigen binding fragment thereof comprises a VH of SEQ ID NO: 160 and a VL of SEQ ID NO: 118. In some embodiments, the antibody or antigen binding fragment thereof comprises a VH of SEQ ID NO: 160 and a VL of SEQ ID NO: 119. In some embodiments, the antibody or antigen binding fragment thereof comprises a VH of SEQ ID NO: 160 and a VL of SEQ ID NO: 120. In some embodiments, the antibody or antigen binding fragment thereof comprises a VH of SEQ ID NO: 160 and a VL of SEQ ID NO: 121. In some embodiments, the antibody or antigen binding fragment thereof comprises a VH of SEQ ID NO: 160 and a VL of SEQ ID NO: 122. In some embodiments, the antibody or antigen binding fragment thereof comprises a VH of SEQ ID NO: 160 and a VL of SEQ ID NO: 123. In some embodiments, the antibody or antigen binding fragment thereof comprises a VH of SEQ ID NO: 160 and a VL of SEQ ID NO: 124. In some embodiments, the antibody or antigen binding fragment thereof comprises a VH of SEQ ID NO: 160 and a VL of SEQ ID NO: 125. In some embodiments, the antibody or antigen binding fragment thereof comprises a VH of SEQ ID NO: 160 and a VL of SEQ ID NO: 126. In some embodiments, the antibody or antigen binding fragment thereof comprises a VH of SEQ ID NO: 160 and a VL of SEQ ID NO: 127. In some embodiments, the antibody or antigen binding fragment thereof comprises a VH of SEQ ID NO: 160 and a VL of SEQ ID NO: 128. In some embodiments, the antibody or antigen binding fragment thereof comprises a VH of SEQ ID NO: 160 and a VL of SEQ ID NO: 129. In some embodiments, the antibody or antigen binding fragment thereof comprises a VH of SEQ ID NO: 160 and a VL of SEQ ID NO: 130. In some embodiments, the antibody or antigen binding fragment thereof comprises a VH of SEQ ID NO: 160 and a VL of SEQ ID NO: 131. In some embodiments, the antibody or antigen binding fragment thereof comprises a VH of SEQ ID NO: 160 and a VL of SEQ ID NO: 132. In some embodiments, the antibody or antigen binding fragment thereof comprises a VH of SEQ ID NO: 160 and a VL of SEQ ID NO: 133. In some embodiments, the antibody or antigen binding fragment thereof comprises a VH of SEQ ID NO: 160 and a VL of SEQ ID NO: 134. In some embodiments, the antibody or antigen binding fragment thereof comprises a VH of SEQ ID NO: 160 and a VL of SEQ ID NO: 135. In some embodiments, the antibody or antigen binding fragment thereof comprises a VH of SEQ ID NO: 160 and a VL of SEQ ID NO: 136. In some embodiments, the antibody or antigen binding fragment thereof comprises a VH of SEQ ID NO: 160 and a VL of SEQ ID NO: 137. In some embodiments, the antibody or antigen binding fragment thereof comprises a VH of SEQ ID NO: 160 and a VL of SEQ ID NO: 138.

In some embodiments, the antibody or antigen binding fragment thereof comprises a VH of SEQ ID NO: 161 and a VL of SEQ ID NO: 114. In some embodiments, the antibody or antigen binding fragment thereof comprises a VH of SEQ ID NO: 161 and a VL of SEQ ID NO: 116. In some embodiments, the antibody or antigen binding fragment thereof comprises a VH of SEQ ID NO: 161 and a VL of SEQ ID NO: 117. In some embodiments, the antibody or antigen binding fragment thereof comprises a VH of SEQ ID NO: 161 and a VL of SEQ ID NO: 118. In some embodiments, the antibody or antigen binding fragment thereof comprises a VH of SEQ ID NO: 161 and a VL of SEQ ID NO: 119. In some embodiments, the antibody or antigen binding fragment thereof comprises a VH of SEQ ID NO: 161 and a VL of SEQ ID NO: 120. In some embodiments, the antibody or antigen binding fragment thereof comprises a VH of SEQ ID NO: 161 and a VL of SEQ ID NO: 121. In some embodiments, the antibody or antigen binding fragment thereof comprises a VH of SEQ ID NO: 161 and a VL of SEQ ID NO: 122. In some embodiments, the antibody or antigen binding fragment thereof comprises a VH of SEQ ID NO: 161 and a VL of SEQ ID NO: 123. In some embodiments, the antibody or antigen binding fragment thereof comprises a VH of SEQ ID NO: 161 and a VL of SEQ ID NO: 124. In some embodiments, the antibody or antigen binding fragment thereof comprises a VH of SEQ ID NO: 161 and a VL of SEQ ID NO: 125. In some embodiments, the antibody or antigen binding fragment thereof comprises a VH of SEQ ID NO: 161 and a VL of SEQ ID NO: 126. In some embodiments, the antibody or antigen binding fragment thereof comprises a VH of SEQ ID NO: 161 and a VL of SEQ ID NO: 127. In some embodiments, the antibody or antigen binding fragment thereof comprises a VH of SEQ ID NO: 161 and a VL of SEQ ID NO: 128. In some embodiments, the antibody or antigen binding fragment thereof comprises a VH of SEQ ID NO: 161 and a VL of SEQ ID NO: 129. In some embodiments, the antibody or antigen binding fragment thereof comprises a VH of SEQ ID NO: 161 and a VL of SEQ ID NO: 130. In some embodiments, the antibody or antigen binding fragment thereof comprises a VH of SEQ ID NO: 161 and a VL of SEQ ID NO: 131. In some embodiments, the antibody or antigen binding fragment thereof comprises a VH of SEQ ID NO: 161 and a VL of SEQ ID NO: 132. In some embodiments, the antibody or antigen binding fragment thereof comprises a VH of SEQ ID NO: 161 and a VL of SEQ ID NO: 133. In some embodiments, the antibody or antigen binding fragment thereof comprises a VH of SEQ ID NO: 161 and a VL of SEQ ID NO: 134. In some embodiments, the antibody or antigen binding fragment thereof comprises a VH of SEQ ID NO: 161 and a VL of SEQ ID NO: 135. In some embodiments, the antibody or antigen binding fragment thereof comprises a VH of SEQ ID NO: 161 and a VL of SEQ ID NO: 136. In some embodiments, the antibody or antigen binding fragment thereof comprises a VH of SEQ ID NO: 161 and a VL of SEQ ID NO: 137. In some embodiments, the antibody or antigen binding fragment thereof comprises a VH of SEQ ID NO: 161 and a VL of SEQ ID NO: 138.

In some embodiments, the antibody or antigen binding fragment thereof comprises a VH of SEQ ID NO: 162 and a VL of SEQ ID NO: 114. In some embodiments, the antibody or antigen binding fragment thereof comprises a VH of SEQ ID NO: 162 and a VL of SEQ ID NO: 116. In some embodiments, the antibody or antigen binding fragment thereof comprises a VH of SEQ ID NO: 162 and a VL of SEQ ID NO: 117. In some embodiments, the antibody or antigen binding fragment thereof comprises a VH of SEQ ID NO: 162 and a VL of SEQ ID NO: 118. In some embodiments, the antibody or antigen binding fragment thereof comprises a VH of SEQ ID NO: 162 and a VL of SEQ ID NO: 119. In some embodiments, the antibody or antigen binding fragment thereof comprises a VH of SEQ ID NO: 162 and a VL of SEQ ID NO: 120. In some embodiments, the antibody or antigen binding fragment thereof comprises a VH of SEQ ID NO: 162 and a VL of SEQ ID NO: 121. In some embodiments, the antibody or antigen binding fragment thereof comprises a VH of SEQ ID NO: 162 and a VL of SEQ ID NO: 122. In some embodiments, the antibody or antigen binding fragment thereof comprises a VH of SEQ ID NO: 162 and a VL of SEQ ID NO: 123. In some embodiments, the antibody or antigen binding fragment thereof comprises a VH of SEQ ID NO: 162 and a VL of SEQ ID NO: 124. In some embodiments, the antibody or antigen binding fragment thereof comprises a VH of SEQ ID NO: 162 and a VL of SEQ ID NO: 125. In some embodiments, the antibody or antigen binding fragment thereof comprises a VH of SEQ ID NO: 162 and a VL of SEQ ID NO: 126. In some embodiments, the antibody or antigen binding fragment thereof comprises a VH of SEQ ID NO: 162 and a VL of SEQ ID NO: 127. In some embodiments, the antibody or antigen binding fragment thereof comprises a VH of SEQ ID NO: 162 and a VL of SEQ ID NO: 128. In some embodiments, the antibody or antigen binding fragment thereof comprises a VH of SEQ ID NO: 162 and a VL of SEQ ID NO: 129. In some embodiments, the antibody or antigen binding fragment thereof comprises a VH of SEQ ID NO: 162 and a VL of SEQ ID NO: 130. In some embodiments, the antibody or antigen binding fragment thereof comprises a VH of SEQ ID NO: 162 and a VL of SEQ ID NO: 131. In some embodiments, the antibody or antigen binding fragment thereof comprises a VH of SEQ ID NO: 162 and a VL of SEQ ID NO: 132. In some embodiments, the antibody or antigen binding fragment thereof comprises a VH of SEQ ID NO: 162 and a VL of SEQ ID NO: 133. In some embodiments, the antibody or antigen binding fragment thereof comprises a VH of SEQ ID NO: 162 and a VL of SEQ ID NO: 134. In some embodiments, the antibody or antigen binding fragment thereof comprises a VH of SEQ ID NO: 162 and a VL of SEQ ID NO: 135. In some embodiments, the antibody or antigen binding fragment thereof comprises a VH of SEQ ID NO: 162 and a VL of SEQ ID NO: 136. In some embodiments, the antibody or antigen binding fragment thereof comprises a VH of SEQ ID NO: 162 and a VL of SEQ ID NO: 137. In some embodiments, the antibody or antigen binding fragment thereof comprises a VH of SEQ ID NO: 162 and a VL of SEQ ID NO: 138.

In some embodiments, the antibody or antigen binding fragment thereof comprises a VH of SEQ ID NO: 163 and a VL of SEQ ID NO: 114. In some embodiments, the antibody or antigen binding fragment thereof comprises a VH of SEQ ID NO: 163 and a VL of SEQ ID NO: 116. In some embodiments, the antibody or antigen binding fragment thereof comprises a VH of SEQ ID NO: 163 and a VL of SEQ ID NO: 117. In some embodiments, the antibody or antigen binding fragment thereof comprises a VH of SEQ ID NO: 163 and a VL of SEQ ID NO: 118. In some embodiments, the antibody or antigen binding fragment thereof comprises a VH of SEQ ID NO: 163 and a VL of SEQ ID NO: 119. In some embodiments, the antibody or antigen binding fragment thereof comprises a VH of SEQ ID NO: 163 and a VL of SEQ ID NO: 120. In some embodiments, the antibody or antigen binding fragment thereof comprises a VH of SEQ ID NO: 163 and a VL of SEQ ID NO: 121. In some embodiments, the antibody or antigen binding fragment thereof comprises a VH of SEQ ID NO: 163 and a VL of SEQ ID NO: 122. In some embodiments, the antibody or antigen binding fragment thereof comprises a VH of SEQ ID NO: 163 and a VL of SEQ ID NO: 123. In some embodiments, the antibody or antigen binding fragment thereof comprises a VH of SEQ ID NO: 163 and a VL of SEQ ID NO: 124. In some embodiments, the antibody or antigen binding fragment thereof comprises a VH of SEQ ID NO: 163 and a VL of SEQ ID NO: 125. In some embodiments, the antibody or antigen binding fragment thereof comprises a VH of SEQ ID NO: 163 and a VL of SEQ ID NO: 126. In some embodiments, the antibody or antigen binding fragment thereof comprises a VH of SEQ ID NO: 163 and a VL of SEQ ID NO: 127. In some embodiments, the antibody or antigen binding fragment thereof comprises a VH of SEQ ID NO: 163 and a VL of SEQ ID NO: 128. In some embodiments, the antibody or antigen binding fragment thereof comprises a VH of SEQ ID NO: 163 and a VL of SEQ ID NO: 129. In some embodiments, the antibody or antigen binding fragment thereof comprises a VH of SEQ ID NO: 163 and a VL of SEQ ID NO: 130. In some embodiments, the antibody or antigen binding fragment thereof comprises a VH of SEQ ID NO: 163 and a VL of SEQ ID NO: 131. In some embodiments, the antibody or antigen binding fragment thereof comprises a VH of SEQ ID NO: 163 and a VL of SEQ ID NO: 132. In some embodiments, the antibody or antigen binding fragment thereof comprises a VH of SEQ ID NO: 163 and a VL of SEQ ID NO: 133. In some embodiments, the antibody or antigen binding fragment thereof comprises a VH of SEQ ID NO: 163 and a VL of SEQ ID NO: 134. In some embodiments, the antibody or antigen binding fragment thereof comprises a VH of SEQ ID NO: 163 and a VL of SEQ ID NO: 135. In some embodiments, the antibody or antigen binding fragment thereof comprises a VH of SEQ ID NO: 163 and a VL of SEQ ID NO: 136. In some embodiments, the antibody or antigen binding fragment thereof comprises a VH of SEQ ID NO: 163 and a VL of SEQ ID NO: 137. In some embodiments, the antibody or antigen binding fragment thereof comprises a VH of SEQ ID NO: 163 and a VL of SEQ ID NO: 138.

In other embodiments, the antibody or antigen binding fragment thereof comprises a VH having a sequence selected from SEQ ID NOs: 165 and 171-177, and/or a VL having a sequence selected from SEQ ID NOs: 164 and 166-170.

In some embodiments, the antibody or antigen binding fragment thereof comprises a VH of SEQ ID NO: 165 and a VL of SEQ ID NO: 164. In some embodiments, the antibody or antigen binding fragment thereof comprises a VH of SEQ ID NO: 165 and a VL of SEQ ID NO: 166. In some embodiments, the antibody or antigen binding fragment thereof comprises a VH of SEQ ID NO: 165 and a VL of SEQ ID NO: 167. In some embodiments, the antibody or antigen binding fragment thereof comprises a VH of SEQ ID NO: 165 and a VL of SEQ ID NO: 168. In some embodiments, the antibody or antigen binding fragment thereof comprises a VH of SEQ ID NO: 165 and a VL of SEQ ID NO: 169. In some embodiments, the antibody or antigen binding fragment thereof comprises a VH of SEQ ID NO: 165 and a VL of SEQ ID NO: 170.

In some embodiments, the antibody or antigen binding fragment thereof comprises a VH of SEQ ID NO: 171 and a VL of SEQ ID NO: 164. In some embodiments, the antibody or antigen binding fragment thereof comprises a VH of SEQ ID NO: 171 and a VL of SEQ ID NO: 166. In some embodiments, the antibody or antigen binding fragment thereof comprises a VH of SEQ ID NO: 171 and a VL of SEQ ID NO: 167. In some embodiments, the antibody or antigen binding fragment thereof comprises a VH of SEQ ID NO: 171 and a VL of SEQ ID NO: 168. In some embodiments, the antibody or antigen binding fragment thereof comprises a VH of SEQ ID NO: 171 and a VL of SEQ ID NO: 169. In some embodiments, the antibody or antigen binding fragment thereof comprises a VH of SEQ ID NO: 171 and a VL of SEQ ID NO: 170.

In some embodiments, the antibody or antigen binding fragment thereof comprises a VH of SEQ ID NO: 172 and a VL of SEQ ID NO: 164. In some embodiments, the antibody or antigen binding fragment thereof comprises a VH of SEQ ID NO: 172 and a VL of SEQ ID NO: 166. In some embodiments, the antibody or antigen binding fragment thereof comprises a VH of SEQ ID NO: 172 and a VL of SEQ ID NO: 167. In some embodiments, the antibody or antigen binding fragment thereof comprises a VH of SEQ ID NO: 172 and a VL of SEQ ID NO: 168. In some embodiments, the antibody or antigen binding fragment thereof comprises a VH of SEQ ID NO: 172 and a VL of SEQ ID NO: 169. In some embodiments, the antibody or antigen binding fragment thereof comprises a VH of SEQ ID NO: 172 and a VL of SEQ ID NO: 170.

In some embodiments, the antibody or antigen binding fragment thereof comprises a VH of SEQ ID NO: 173 and a VL of SEQ ID NO: 164. In some embodiments, the antibody or antigen binding fragment thereof comprises a VH of SEQ ID NO: 173 and a VL of SEQ ID NO: 166. In some embodiments, the antibody or antigen binding fragment thereof comprises a VH of SEQ ID NO: 173 and a VL of SEQ ID NO: 167. In some embodiments, the antibody or antigen binding fragment thereof comprises a VH of SEQ ID NO: 173 and a VL of SEQ ID NO: 168. In some embodiments, the antibody or antigen binding fragment thereof comprises a VH of SEQ ID NO: 173 and a VL of SEQ ID NO: 169. In some embodiments, the antibody or antigen binding fragment thereof comprises a VH of SEQ ID NO: 173 and a VL of SEQ ID NO: 170.

In some embodiments, the antibody or antigen binding fragment thereof comprises a VH of SEQ ID NO: 174 and a VL of SEQ ID NO: 164. In some embodiments, the antibody or antigen binding fragment thereof comprises a VH of SEQ ID NO: 174 and a VL of SEQ ID NO: 166. In some embodiments, the antibody or antigen binding fragment thereof comprises a VH of SEQ ID NO: 174 and a VL of SEQ ID NO: 167. In some embodiments, the antibody or antigen binding fragment thereof comprises a VH of SEQ ID NO: 174 and a VL of SEQ ID NO: 168. In some embodiments, the antibody or antigen binding fragment thereof comprises a VH of SEQ ID NO: 174 and a VL of SEQ ID NO: 169. In some embodiments, the antibody or antigen binding fragment thereof comprises a VH of SEQ ID NO: 174 and a VL of SEQ ID NO: 170.

In some embodiments, the antibody or antigen binding fragment thereof comprises a VH of SEQ ID NO: 175 and a VL of SEQ ID NO: 164. In some embodiments, the antibody or antigen binding fragment thereof comprises a VH of SEQ ID NO: 175 and a VL of SEQ ID NO: 166. In some embodiments, the antibody or antigen binding fragment thereof comprises a VH of SEQ ID NO: 175 and a VL of SEQ ID NO: 167. In some embodiments, the antibody or antigen binding fragment thereof comprises a VH of SEQ ID NO: 175 and a VL of SEQ ID NO: 168. In some embodiments, the antibody or antigen binding fragment thereof comprises a VH of SEQ ID NO: 175 and a VL of SEQ ID NO: 169. In some embodiments, the antibody or antigen binding fragment thereof comprises a VH of SEQ ID NO: 175 and a VL of SEQ ID NO: 170.

In some embodiments, the antibody or antigen binding fragment thereof comprises a VH of SEQ ID NO: 176 and a VL of SEQ ID NO: 164. In some embodiments, the antibody or antigen binding fragment thereof comprises a VH of SEQ ID NO: 176 and a VL of SEQ ID NO: 166. In some embodiments, the antibody or antigen binding fragment thereof comprises a VH of SEQ ID NO: 176 and a VL of SEQ ID NO: 167. In some embodiments, the antibody or antigen binding fragment thereof comprises a VH of SEQ ID NO: 176 and a VL of SEQ ID NO: 168. In some embodiments, the antibody or antigen binding fragment thereof comprises a VH of SEQ ID NO: 176 and a VL of SEQ ID NO: 169. In some embodiments, the antibody or antigen binding fragment thereof comprises a VH of SEQ ID NO: 176 and a VL of SEQ ID NO: 170.

In some embodiments, the antibody or antigen binding fragment thereof comprises a VH of SEQ ID NO: 177 and a VL of SEQ ID NO: 164. In some embodiments, the antibody or antigen binding fragment thereof comprises a VH of SEQ ID NO: 177 and a VL of SEQ ID NO: 166. In some embodiments, the antibody or antigen binding fragment thereof comprises a VH of SEQ ID NO: 177 and a VL of SEQ ID NO: 167. In some embodiments, the antibody or antigen binding fragment thereof comprises a VH of SEQ ID NO: 177 and a VL of SEQ ID NO: 168. In some embodiments, the antibody or antigen binding fragment thereof comprises a VH of SEQ ID NO: 177 and a VL of SEQ ID NO: 169. In some embodiments, the antibody or antigen binding fragment thereof comprises a VH of SEQ ID NO: 177 and a VL of SEQ ID NO: 170.

In some embodiments, the antibody or antigen binding fragment thereof provided herein comprises (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1, Lys 12, Val 20, Tyr 27, Thr 28, Phe 29, Thr 30, Arg 38, Met 48, Arg 67, Val 68, Ala 72, Ser 77, Ala 79, Met 81, Leu 83 and Val 117; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77 and Asp 87.

In some embodiments, the antibody or antigen binding fragment thereof comprises (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ala 72 with Thr, Ser 77 with Asp, Ala 79 with Val, Met 81 with Leu, Leu 83 with Phe and Val 117 with Leu; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Ala, Thr 48 with Gly, Ser 51 with Gly, Trp 59 with Gly, Thr 60 with Val, Leu 77 with Ile, and Asp 87 with Ile.

In some embodiments, the antibody or antigen binding fragment thereof comprises (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln 1, Lys 12, Val 20, Tyr 27, Thr 28, Phe 29, Thr 30, Arg 38, Met 48, Arg 67, Val 68, Ile 70, Ala 72, Ser 77, Met 81, and Val 117; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77, and Asp 87.

In some embodiments, the antibody or antigen binding fragment thereof comprises (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ile 70 with Leu, Ala 72 with Thr, Ser 77 with Asn, Met 81 with Leu, and Val 117 with Leu; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Thr, Thr 48 with Gly, Ser 51 with Gly, Trp 59 with Gly, Thr 60 with Val, Leu 77 with Ile, and Asp 87 with Ile.

In yet another aspect, provided herein are antibodies that compete with one of the antibody or antigen binding fragment thereof described above. Such antibodies may also bind to the same epitope as one of the above mentioned antibodies, or an overlapping epitope. Antibodies and fragments that compete with or bind to the same epitope as the above-mentioned antibodies are expected to show similar functional properties. The exemplified antigen binding proteins and fragments include those with the VH regions, and CDRs provided herein, including those in Tables 8, 10, and 11-12, and FIGS. 10-13.

In certain embodiments, an antibody described herein or an antigen-binding fragment thereof comprises amino acid sequences with certain percent identity relative to any one of antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HV10b and 144L249B LV9 HV10c described in Section 6 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 23, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 51, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 23 and the amino acid sequence of SEQ ID NO: 51 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 23, wherein the antibody immunospecifically binds to IL-36α and/or IL-36γ.

In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 51, wherein the antibody immunospecifically binds to IL-36α and/or IL-36γ.

In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 23 and SEQ ID NO: 51 respectively, wherein the antibody immunospecifically binds to IL-36α and/or IL-36y. In specific embodiments, such an antibody comprises CDRs (e.g., VH CDRs 1-3, VL CDRs 1-3) identical to the CDRs (e.g., VH CDRs 1-3, VL CDRs 1-3) of antibody 144D464A.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 27, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 55, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 27 and the amino acid sequence of SEQ ID NO: 55 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 27, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 55, wherein the antibody immunospecifically binds to IL-36α and/or IL-36γ. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 27 and SEQ ID NO: 55 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144L249B.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 31, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 55, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 31 and the amino acid sequence of SEQ ID NO: 55 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 31, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 55, wherein the antibody immunospecifically binds to IL-36α and/or IL-36γ. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 31 and SEQ ID NO: 55 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144L124B or 144L180A.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 35, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 55, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 35 and the amino acid sequence of SEQ ID NO: 55 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 35, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 55, wherein the antibody immunospecifically binds to IL-36α and/or IL-36y. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 35 and SEQ ID NO: 55 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144L133B.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 39, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 59, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 39 and the amino acid sequence of SEQ ID NO: 59 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 39, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 59, wherein the antibody immunospecifically binds to IL-36α and/or IL-36y. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 39 and SEQ ID NO: 59 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144L472A.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 43, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 63, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 43 and the amino acid sequence of SEQ ID NO: 63 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 43, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 63, wherein the antibody immunospecifically binds to IL-36α and/or IL-36γ. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 43 and SEQ ID NO: 63 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144D666C.

In certain embodiments, an antibody provided herein or an antigen-binding fragment thereof comprises a VH region having at least 80%, at least 85%, at least 90%, at least 95%, or at least 98% sequence identity to the amino acid sequence of SEQ ID NO: 47, wherein the antibody immunospecifically binds to IL-36α and/or IL-36γ.

In certain embodiments, an antibody provided herein or an antigen-binding fragment thereof comprises a VL region having at least 80%, at least 85%, at least 90%, at least 95%, or at least 98% sequence identity to the amino acid sequence of SEQ ID NO: 67, wherein the antibody immunospecifically binds to IL-36α and/or IL-36γ.

In certain embodiments, an antibody provided herein or an antigen-binding fragment thereof comprises a VH region and a VL region having at least 80%, at least 85%, at least 90%, at least 95%, or at least 98% sequence identity to the amino acid sequence of SEQ ID NO: 47 and the amino acid sequence of SEQ ID NO: 67 respectively, wherein the antibody immunospecifically binds to IL-36α and/or IL-36γ.

In certain embodiments, an antibody provided herein or an antigen-binding fragment thereof comprises a VH region comprising VH framework regions having at least 80%, at least 85%, at least 90%, at least 95%, or at least 98% sequence identity to the amino acid sequence of the framework regions of SEQ ID NO: 47, wherein the antibody immunospecifically binds to IL-36α and/or IL-36γ. In certain embodiments, an antibody provided herein or an antigen-binding fragment thereof comprises a VL region comprising VL framework regions having at least 80%, at least 85%, at least 90%, at least 95%, or at least 98% sequence identity to the amino acid sequence of the framework regions of SEQ ID NO: 67, wherein the antibody immunospecifically binds to IL-36α and/or IL-36γ. In certain embodiments, an antibody provided herein or an antigen-binding fragment thereof comprises a VH region and a VL region comprising VH framework regions and VL framework regions having at least 80%, at least 85%, at least 90%, at least 95%, or at least 98% sequence identity to the amino acid sequence of the framework regions of SEQ ID NO: 47 and SEQ ID NO: 67 respectively, wherein the antibody immunospecifically binds to IL-36α and/or IL-36γ. In specific embodiments, such an antibody comprises CDRs (e.g., VH CDRs 1-3, VL CDRs 1-3) identical to the CDRs (e.g., VH CDRs 1-3, VL CDRs 1-3) of antibody 144J171G.

### 5.2.2 Polyclonal Antibodies

The antibodies of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include an IL-36α or IL-36γ polypeptide or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly (I:C), Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for anti- IL-36α or IL-36γ antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 5.2.3 Monoclonal Antibodies

The antibodies of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, Va.), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Plückthun, 1992, Immunol. Revs. 130:151-88.

In some embodiments, an antibody that binds an IL-36α and/or IL-36γ epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2× SSC/0.1% SDS at about 50-65° C.), under highly stringent conditions (e.g., hybridization to filter-bound nucleic acid in 6× SSC at about 45° C. followed by one or more washes in 0.1X SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds an IL-36α and/or IL-36γ epitope comprises an amino acid sequence of a VH CDR or an amino acid sequence of a VL CDR encoded by a nucleotide sequence that hybridizes to the complement of a nucleotide sequence encoding any one of the VH CDRs and/or VL CDRs depicted in Table 11 and Table 12 under stringent conditions (e.g., hybridization to filter-bound DNA in 6× SSC at about 45° C. followed by one or more washes in 0.2× SSC/0.1% SDS at about 50-65° C.), under highly stringent conditions (e.g., hybridization to filter-bound nucleic acid in 6× SSC at about 45° C. followed by one or more washes in 0.1× SSC/0.2% SDS at about 68° C.), or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In phage display methods, functional antibody domains are displayed on the surface of phage particles which carry the polynucleotide sequences encoding them. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, IL-36α and/or IL-36γ (e.g., an IL-36α and/or IL-36γ polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-IL-36α and/or IL-36γ antibodies can be obtained by designing a suitable antigen screening procedure to select for the phage clone of interest followed by construction of a full length antibody clone using VH and/or VL sequences (e.g., the Fv sequences), or various CDR sequences from VH and VL sequences, from the phage clone of interest and suitable constant region (e.g., Fc) sequences described in Kabat et al., supra.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies or antigen binding fragments produced using techniques such as those described herein can be isolated using standard, well known techniques. For example, antibodies or antigen binding fragments can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized.

### 5.2.4 Antibody Fragments

The present disclosure provides antibodies and antibody fragments that bind to IL-36α and/or IL-36γ. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to an IL-36α and/or IL-36γ epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to an IL-36α and/or IL-36γ epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes) ; and a tetrabody (e.g., a tetramerized scFv, formed in a manner similar to a diabody, but in which four antigen-binding domains are created in a single complex; the four antigen binding domains may be directed towards the same or different epitopes).

### 5.2.5 Humanized Antibodies

The antibodies described herein can, for example, include humanized antibodies, e.g., deimmunized or composite human antibodies.

A humanized antibody can comprise human framework region and human constant region sequences.

For example, a humanized antibody can comprise human constant region sequences. In certain embodiments, a humanized antibody can be selected from any class of immunoglobulins, including IgM, IgG, IgD, IgA and IgE, and any isotype, including IgG1, IgG2, IgG3 and IgG4. In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s- 5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, antibodies provided herein can be humanized antibodies that bind IL-36α and/or IL-36γ, including human and/or cynomolgus macaque IL-36α and/or IL-36γ. For example, humanized antibodies of the present disclosure may comprise one or more CDRs as shown in Table 11 and Table 12. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009;525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In a specific embodiment, a humanized antibody provided herein is generated using the method described in Section 6 below.

### 5.2.6 Human Antibodies

In specific embodiments, the antibody is a fully human anti-human antibody. Fully human antibodies may be produced by any method known in the art. Human anti-IL-36 antibodies provided herein, e.g., dual antagonist antibody binding to IL-36α and IL-36γ, can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 5.2.7 Multipecific Antibodies

Multispecific antibodies such as bispecific antibodies are monoclonal antibodies that have binding specificities for at least two different antigens. In certain embodiments, the multispecific antibodies can be constructed based on the sequences of the antibodies provided herein, e.g., the CDR sequences listed in Table 11 and Table 12. In certain embodiments, the multispecific antibodies provided herein are bispecific antibodies. In certain embodiments, bispecific antibodies are human or humanized antibodies. In certain embodiments, one of the binding specificities is for IL-36α and/or IL-36γ and the other is for any other antigen. In certain embodiments, one of the binding specificities is for IL-36α and IL-36γ and the other is for any other antigen. In some embodiments, one of the binding specificities is for IL-36α and/or IL-36γ, and the other is for another antigen such as a cytokine or chemokine. In some embodiments, one of the binding specificities is for IL-36α and IL-36γ, and the other is for another antigen such as a cytokine or chemokine. In certain embodiments, bispecific antibodies may bind to two different epitopes of IL-36α and/or IL-36γ. Bispecific antibodies can be prepared as full length antibodies or antibody fragments (e.g., F(ab′)2 bispecific antibodies).

Methods for making multipecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

### 5.2.8 Multivalent Antibodies

A multivalent antibody may be internalized (and/or catabolized) faster than a bivalent antibody by a cell expressing an antigen to which the antibodies bind. The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 5.2.9 Fc Engineering

It may be desirable to modify an antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody.

To increase the serum half life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 5.2.10 Alternative Binding Agents

The present disclosure encompasses non-immunoglobulin binding agents that specifically bind to the same epitope as an antibody disclosed herein. In some embodiments, a non-immunoglobulin binding agent is identified as an agent that displaces or is displaced by an antibody of the present disclosure in a competitive binding assay. These alternative binding agents may include, for example, any of the engineered protein scaffolds known in the art. Such scaffolds may comprise one or more CDRs as shown in Tables 11-12. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human γ-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### 5.2.11 Antibody Variants

In some embodiments, amino acid sequence modification(s) of the antibodies or antigen binding fragments that bind to IL-36α and/or IL-36γ provided herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or antigen binding fragment thereof that binds to an IL-36α and/or IL-36γ epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for examples, antibodies 144D464A, 144L249B, 144L124B, 144L133B, 144L180A, 144L472A, 144D666C, 144J171G, 144D464A LV7a HV10b, 144D464A LV9are HV10b, 144D464A LV10re HV10b, 144D464A LV11re HV10b, 144L249B LV7a HV11, 144L249B LV9 HV11, 144L249B LV9 HVi0b and 144L249B LV9 HV10c described in Section 6 below.

In one embodiment, an antibody or antigen-binding fragment thereof that binds to an IL-36α and/or IL-36γ epitope comprises an amino acid sequence that is at least 35% identical to the amino acid sequence of an antibody described herein. In one embodiment, an antibody or antigen-binding fragment thereof that binds to an IL-36α and/or IL-36γ epitope comprises an amino acid sequence that is at least 40% identical to the amino acid sequence of an antibody described herein. In one embodiment, an antibody or antigen-binding fragment thereof that binds to an IL-36α and/or IL-36γ epitope comprises an amino acid sequence that is at least 45% identical to the amino acid sequence of an antibody described herein. In one embodiment, an antibody or antigen-binding fragment thereof that binds to an IL-36α and/or IL-36γ epitope comprises an amino acid sequence that is at least 50% identical to the amino acid sequence of an antibody described herein. In one embodiment, an antibody or antigen-binding fragment thereof that binds to an IL-36α and/or IL-36γ epitope comprises an amino acid sequence that is at least 55% identical to the amino acid sequence of an antibody described herein. In one embodiment, an antibody or antigen-binding fragment thereof that binds to an IL-36α and/or IL-36γ epitope comprises an amino acid sequence that is at least 60% identical to the amino acid sequence of an antibody described herein. In one embodiment, an antibody or antigen-binding fragment thereof that binds to an IL-36α and/or IL-36γ epitope comprises an amino acid sequence that is at least 65% identical to the amino acid sequence of an antibody described herein. In one embodiment, an antibody or antigen-binding fragment thereof that binds to an IL-36α and/or IL-36γ epitope comprises an amino acid sequence that is at least 70% identical to the amino acid sequence of an antibody described herein. In one embodiment, an antibody or antigen-binding fragment thereof that binds to an IL-36α and/or IL-36γ epitope comprises an amino acid sequence that is at least 75% identical to the amino acid sequence of an antibody described herein. In one embodiment, an antibody or antigen-binding fragment thereof that binds to an IL-36α and/or IL-36γ epitope comprises an amino acid sequence that is at least 80% identical to the amino acid sequence of an antibody described herein. In one embodiment, an antibody or antigen-binding fragment thereof that binds to an IL-36α and/or IL-36γ epitope comprises an amino acid sequence that is at least 90% identical to the amino acid sequence of an antibody described herein. In one embodiment, an antibody or antigen-binding fragment thereof that binds to an IL-36α and/or IL-36γ epitope comprises an amino acid sequence that is at least 95% identical to the amino acid sequence of an antibody described herein. In one embodiment, an antibody or antigen-binding fragment thereof that binds to an IL-36α and/or IL-36γ epitope comprises an amino acid sequence that is at least 99% identical to the amino acid sequence of an antibody described herein.

In some embodiments, provided herein is an antibody or antigen binding fragment thereof that binds to an IL-36α and/or IL-36γ epitope and comprises a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Table 8, and/or a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Table 10.

In some embodiments, provided herein is an antibody or antigen binding fragment thereof that binds to an IL-36α and/or IL-36γ epitope and comprises a VH region comprising an amino acid sequence that is at least 35% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 40% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 45% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 50% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 55% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 60% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 65% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 70% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 75% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 80% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 85% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 90% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 95% identical to an amino acid sequence depicted in Table 8. In some embodiments, provided herein is an antibody or antigen binding fragment thereof that binds to an IL-36α and/or IL-36γ epitope and comprises a VH region comprising an amino acid sequence that is at least 99% identical to an amino acid sequence depicted in Table 8.

In some embodiments, provided herein is an antibody or antigen binding fragment thereof that binds to an IL-36α and/or IL-36γ epitope and comprises a VL region comprising an amino acid sequence that is at least 35% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 40% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 45% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 50% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 55% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 60% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 65% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 70% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 75% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 80% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 85% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 90% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 95% identical to an amino acid sequence depicted in Table 10. In some embodiments, provided herein is an antibody or antigen binding fragment thereof that binds to an IL-36α and/or IL-36γ epitope and comprises a VL region comprising an amino acid sequence that is at least 99% identical to an amino acid sequence depicted in Table 10.

In yet another embodiment, an antibody or antigen binding fragment thereof that binds to an IL-36α and/or IL-36γ epitope comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Table 11 and/or a VL CDR amino acid sequence depicted in Table 12.

In yet another embodiment, an antibody or antigen binding fragment thereof that binds to an IL-36α and/or IL-36γ epitope comprises a VH CDR and/or a VL CDR amino acid sequence that is at least 35% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 40% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 45% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 50% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 55% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 60% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 65% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 70% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 75% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 80% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 85% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 90% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 95% identical to a VH CDR amino acid sequence depicted in Table 11 and/or a VL CDR amino acid sequence depicted in Table 12. In yet another embodiment, an antibody or antigen binding fragment thereof that binds to an IL-36α and/or IL-36γ epitope comprises a VH CDR and/or a VL CDR amino acid sequence that is at least 99% identical to a VH CDR amino acid sequence depicted in Table 11 and/or a VL CDR amino acid sequence depicted in Table 12.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-IL-36α and/or IL-36γ antibody variant DNA.

Any cysteine residue not involved in maintaining the proper conformation of the antibody provided herein also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In some embodiments, an antibody molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, for example, by in vitro determination of T-cell epitopes or in silico prediction of such epitopes, as known in the art. Once the critical residues for T-cell epitope function have been identified, mutations can be made to remove immunogenicity and retain antibody activity. For review, see, for example, Jones et al., 2009, Methods in Molecular Biology 525:405-23.

### 5.2.12 In vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10″ in size (see, e.g., U.S. Pat. Publication 2003/0186374; and Blaise et aL, 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, the antibodies can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 5.2.13 Antibody Modifications

Covalent modifications of the antibodies binding to IL-36α and/or IL-36γ provided herein are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody provided herein included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody of the present disclosure may also be modified to form chimeric molecules comprising the antibody fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to IL-36α and/or IL-36γ and a heterologous polypeptide.

Also provided herein are panels of antibodies that bind to an IL-36α and/or IL-36γ antigen. In specific embodiments, the panels of antibodies have different association rates, different dissociation rates, different affinities for an IL-36α and/or IL-36γ antigen, and/or different specificities for an IL-36α and/or IL-36γ antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 5.2.14 Immunoconjugates

The present disclosure also provides conjugates comprising any one of the antibodies of the present disclosure covalently bound by a synthetic linker to one or more non-antibody agents.

In some embodiments, antibodies provided herein are conjugated or recombinantly fused, e.g., to a therapeutic agent (e.g., a cytotoxic agent) or a diagnostic or detectable molecule. The conjugated or recombinantly fused antibodies can be useful, for example, for treating or preventing a disease or disorder such as an IL-36-mediated disease. The conjugated or recombinantly fused antibodies can be useful, for example, for monitoring or prognosing the onset, development, progression, and/or severity of an IL-36-mediated disease.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (131I, 125I, 123I, and 121I,), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113 Sn, or 117Sn; positron emitting metals using various positron emission tomographies; and non-radioactive paramagnetic metal ions.

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., CDR1, CDR2, and/or CDR3) and a heterologous protein, polypeptide, or peptide. In one embodiment, the heterologous protein, polypeptide, or peptide that the antibody is fused to is useful for targeting the antibody to a particular cell type.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-IVIES, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the ϵ-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50): 12047-57).

### 5.3 Polynucleotides

In certain embodiments, the disclosure encompasses polynucleotides that encode the antibodies described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag supplied by a vector that allows efficient purification of the polypeptide fused to the marker in the case of a bacterial host. In some embodiments, a marker is used in conjunction with other affinity tags.

In certain embodiments, the polynucleotide provided herein is selected from the polynucleotides listed in Tables 3-6 below or any combinaions thereof. In certain embodiment, the polynucleotide comprises a nucleotide sequence of SEQ ID NO: 20. In certain embodiment, the polynucleotide comprises a nucleotide sequence of SEQ ID NO: 24. In some embodiments, the polynucleotide comprises a nucleotide sequence of SEQ ID NO: 28. In other embodiments, the polynucleotide comprises a nucleotide sequence of SEQ ID NO: 32. In yet other embodiments, the polynucleotide comprises a nucleotide sequence of SEQ ID NO: 36. In yet other embodiments, the polynucleotide comprises a nucleotide sequence of SEQ ID NO: 40. In yet other embodiments, the polynucleotide comprises a nucleotide sequence of SEQ ID NO: 44. In yet other embodiments, the polynucleotide comprises a nucleotide sequence of SEQ ID NO: 22. In yet other embodiments, the polynucleotide comprises a nucleotide sequence of SEQ ID NO: 26. In yet other embodiments, the polynucleotide comprises a nucleotide sequence of SEQ ID NO: 30. In yet other embodiments, the polynucleotide comprises a nucleotide sequence of SEQ ID NO: 34. In yet other embodiments, the polynucleotide comprises a nucleotide sequence of SEQ ID NO: 38. In yet other embodiments, the polynucleotide comprises a nucleotide sequence of SEQ ID NO: 42. In yet other embodiments, the polynucleotide comprises a nucleotide sequence of SEQ ID NO: 46. In yet other embodiments, the polynucleotide comprises a nucleotide sequence of SEQ ID NO: 48. In yet other embodiments, the polynucleotide comprises a nucleotide sequence of SEQ ID NO: 52. In yet other embodiments, the polynucleotide comprises a nucleotide sequence of SEQ ID NO: 56. In yet other embodiments, the polynucleotide comprises a nucleotide sequence of SEQ ID NO: 60. In yet other embodiments, the polynucleotide comprises a nucleotide sequence of SEQ ID NO: 64. In yet other embodiments, the polynucleotide comprises a nucleotide sequence of SEQ ID NO: 50. In yet other embodiments, the polynucleotide comprises a nucleotide sequence of SEQ ID NO: 54. In yet other embodiments, the polynucleotide comprises a nucleotide sequence of SEQ ID NO: 58. In yet other embodiments, the polynucleotide comprises a nucleotide sequence of SEQ ID NO: 62. In yet other embodiments, the polynucleotide comprises a nucleotide sequence of SEQ ID NO: 66.

In some embodiments, the polynucleotide described herein encode any polypeptide provided herein, including for example those described in Section 6 below and FIGS. 10-13.

The present disclosure further relates to variants of the polynucleotides described herein, wherein the variant encodes, for example, fragments, analogs, and/or derivatives of a polypeptide. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an antibody or antigen binding fragment thereof described herein.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to a polynucleotide listed in Tables 3-6 below.

In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 20. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 24. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 28. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 32. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 36. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 40. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 44. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 22. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 26. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 30. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 34. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 38. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 42. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 46. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 48. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 52. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 56. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 60. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 64. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 50. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 54. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 58. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 62. In certain embodiments, the present disclosure provides a polynucleotide comprising a a nucleotide sequence at least about 80% identical, at least about 85% identical, at least about 90% identical, at least about 95% identical, and in some embodiments, at least about 96%, 97%, 98% or 99% identical to the polynucleotide of SEQ ID NO: 66.

In certain embodiments, a polynucleotide is isolated. In certain embodiments, a polynucleotide is substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises one or more expression vectors comprising polynucleotide molecules. In some embodiments, a host cell comprises a polynucleotide molecule. In some embodiments, a host cell comprises one or more polynucleotide molecules. Construction of the vectors provided herein is exemplified in Section 6 below.

### 5.4 Methods of Making the Antibodies

In yet another aspect, provided herein are methods for making the various antibodies or antigen binding fragments provided herein.

Recombinant expression of an antibody provided herein (e.g., a full-length antibody, heavy and/or light chain of an antibody, or a single chain antibody provided herein) that immunospecifically binds to an IL-36 antigen (e.g., IL-36α and/or IL-36β) requires construction of an expression vector containing a polynucleotide that encodes the antibody. Once a polynucleotide encoding an antibody molecule, heavy or light chain of an antibody, or fragment thereof (such as, but not necessarily, containing the heavy and/or light chain variable domain) provided herein has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule provided herein, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody provided herein. Thus, also provided herein are host cells containing a polynucleotide encoding an antibody provided herein or fragments thereof, or a heavy or light chain thereof, or fragment thereof, or a single chain antibody provided herein, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In some embodiments, antibodies provided herein are produced in CHO cells. In a specific embodiment, the expression of nucleotide sequences encoding antibodies provided herein which immunospecifically bind to an IL-36 antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells. In some embodiments, fully human monoclonal antibodies provided herein are produced in mammalian cells, such as CHO cells.

For long-term, high-yield production of recombinant proteins, stable expression can be utilized. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O′Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors provided herein, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains may comprise cDNA or genomic DNA.

Once an antibody molecule provided herein has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies provided herein can be fused to heterologous polypeptide sequences described herein or otherwise known in the art to facilitate purification.

### 5.5 Pharmaceutical Compositions

In one aspect, the present disclosure further provides pharmaceutical compositions comprising at least one antibody or antigen binding fragment thereof of the present disclosure. In some embodiments, a pharmaceutical composition comprises therapeutically effective amount of an antibody or antigen binding fragment thereof provided herein and a pharmaceutically acceptable excipient.

Pharmaceutical compositions comprising an antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek etal., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6 Methods of Using the Antibodies and Pharmaceutical Compositions

In one aspect, provided herein is a method of attenuating an activity of IL-36α and/or IL-36γ on a cell, comprising exposing the cell to an effective amount of an antibody or antigen binding fragment thereof provided herein.

In some embodiments, the antibody provided herein attenuates an IL-36α and/or IL-36γ activity by at least about 10%. In some embodiments, the antibody provided herein attenuates an IL-36α and/or IL-36γ activity by at least about 20%. In some embodiments, the antibody provided herein attenuates an IL-36α and/or IL-36γ activity by at least about 30%. In some embodiments, the antibody provided herein attenuates an IL-36α and/or IL-36γ activity by at least about 40%. In some embodiments, the antibody provided herein attenuates an IL-36α and/or IL-36γ activity by at least about 50%. In some embodiments, the antibody provided herein attenuates an IL-36α and/or IL-36γ activity by at least about 60%. In some embodiments, the antibody provided herein attenuates an IL-36α and/or IL-36γ activity by at least about 70%. In some embodiments, the antibody provided herein attenuates an IL-36α and/or IL-36γ activity by at least about 80%. In some embodiments, the antibody provided herein attenuates an IL-36α and/or IL-36γ activity by at least about 90%. In some embodiments, the antibody provided herein attenuates an IL-36α and/or IL-36γ activity by at least about 95%. In certain embodiments, the antibody described herein can attenuate (e.g., partially attenuate) an IL-36α and/or IL-36γ activity by at least about 15% to about 65%. In certain embodiments, the antibody described herein can attenuate (e.g., partially attenuate) an IL-36α and/or IL-36γ activity by at least about 20% to about 65%. In certain embodiments, the antibody described herein can attenuate (e.g., partially attenuate) an IL-36α and/or IL-36γ activity by at least about 30% to about 65%.

A non-limiting example of an IL-36α and/or IL-36γ activity is IL-36α and/or IL-36γ mediated signaling. Thus, in certain embodiments, provided herein is a method of attenuating (e.g., partially attenuating) IL-36α and/or IL-36γ mediated signaling in a cell, comprising exposing the cell to an effective amount of an antibody or antigen binding fragment thereof provided herein.

In some embodiments, the antibody provided herein attenuates IL-36α and/or IL-36γ mediated signaling by at least about 10%. In some embodiments, the antibody provided herein attenuates IL-36α and/or IL-36γ mediated signaling by at least about 20%. In some embodiments, the antibody provided herein attenuates IL-36α and/or IL-36γ mediated signaling by at least about 30%. In some embodiments, the antibody provided herein attenuates IL-36a and/or IL-36γ mediated signaling by at least about 40%. In some embodiments, the antibody provided herein attenuates IL-36α and/or IL-36γ mediated signaling by at least about 50%. In some embodiments, the antibody provided herein attenuates IL-36α and/or IL-36γ mediated signaling by at least about 60%. In some embodiments, the antibody provided herein attenuates IL-36α and/or IL-36γ mediated signaling by at least about 70%. In some embodiments, the antibody provided herein attenuates IL-36α and/or IL-36γ mediated signaling by at least about 80%. In some embodiments, the antibody provided herein attenuates IL-36α and/or IL-36y mediated signaling by at least about 90%. In some embodiments, the antibody provided herein attenuates IL-36α and/or IL-36γ mediated signaling by at least about 95%. In certain embodiments, the antibody described herein can attenuate (e.g., partially attenuate) IL-36α and/or IL-36γ mediated signaling by at least about 15% to about 65%. In certain embodiments, the antibody described herein can attenuate (e.g., partially attenuate) IL-36α and/or IL-36γ mediated signaling by at least about 20% to about 65%. In certain embodiments, the antibody described herein can attenuate (e.g., partially attenuate) IL-36α and/or IL-36γ mediated signaling by at least about 30% to about 65%.

Another non-limiting example of an IL-36α and/or IL-36γ activity is binding to IL-36 receptor. Thus, in certain embodiments, provided herein is a method of attenuating (e.g., partially attenuating) the binding of IL-36α and/or IL-36γ to an IL-36 receptor on a cell, comprising exposing the cell to an effective amount of an antibody or antigen binding fragment thereof provided herein.

In some embodiments, the antibody provided herein attenuates the binding of IL-36α and/or IL-36γ to an IL-36 receptor by at least about 10%. In some embodiments, the antibody provided herein attenuates the binding of IL-36α and/or IL-36γ to an IL-36 receptor by at least about 20%. In some embodiments, the antibody provided herein attenuates the binding of IL-36α a and/or IL-36γ to an IL-36 receptor by at least about 30%. In some embodiments, the antibody provided herein attenuates the binding of IL-36α and/or IL-36γ to an IL-36 receptor by at least about 40%. In some embodiments, the antibody provided herein attenuates the binding of IL-36α and/or IL-36γ to an IL-36 receptor by at least about 50%. In some embodiments, the antibody provided herein attenuates the binding of IL-36α and/or IL-36γ to an IL-36 receptor by at least about 60%. In some embodiments, the antibody provided herein attenuates the binding of IL-36α and/or IL-36γ to an IL-36 receptor by at least about 70%. In some embodiments, the antibody provided herein attenuates the binding of IL-36α and/or IL-36γ to an IL-36 receptor by at least about 80%. In some embodiments, the antibody provided herein attenuates the binding of IL-36α and/or IL-36γ to an IL-36 receptor by at least about 90%. In some embodiments, the antibody provided herein attenuates the binding of IL-36α and/or IL-36γ to an IL-36 receptor by at least about 95%. In certain embodiments, the antibody described herein can attenuate (e.g., partially attenuate) the binding of IL-36α and/or IL-36γ to an IL-36 receptor by at least about 15% to about 65%. In certain embodiments, the antibody described herein can attenuate (e.g., partially attenuate) the binding of IL-36α and/or IL-36γ to an IL-36 receptor by at least about 20% to about 65%. In certain embodiments, the antibody described herein can attenuate (e.g., partially attenuate) the binding of IL-36α and/or IL-36γ to an IL-36 receptor by at least about 30% to about 65%.

Yet another non-limiting example of an IL-36α and/or IL-36γ activity is signaling mediated by an IL-36 receptor. Thus, in certain embodiments, provided herein is a method of attenuating (e.g., partially attenuating) IL-36 receptor mediated signaling in a cell, comprising exposing the cell to an effective amount of an antibody or antigen binding fragment thereof provided herein.

In some embodiments, the antibody provided herein attenuates IL-36 receptor mediated signaling by at least about 10%. In some embodiments, the antibody provided herein attenuates IL-36 receptor mediated signaling by at least about 20%. In some embodiments, the antibody provided herein attenuates IL-36 receptor mediated signaling by at least about 30%. In some embodiments, the antibody provided herein attenuates IL-36 receptor mediated signaling by at least about 40%. In some embodiments, the antibody provided herein attenuates IL-36 receptor mediated signaling by at least about 50%. In some embodiments, the antibody provided herein attenuates IL-36 receptor mediated signaling by at least about 60%. In some embodiments, the antibody provided herein attenuates IL-36 receptor mediated signaling by at least about 70%. In some embodiments, the antibody provided herein attenuates IL-36 receptor mediated signaling by at least about 80%. In some embodiments, the antibody provided herein attenuates IL-36 receptor mediated signaling by at least about 90%. In some embodiments, the antibody provided herein attenuates IL-36 receptor mediated signaling by at least about 95%. In certain embodiments, the antibody described herein can attenuate (e.g., partially attenuate) IL-36 receptor mediated signaling by at least about 15% to about 65%. In certain embodiments, the antibody described herein can attenuate (e.g., partially attenuate) IL-36 receptor mediated signaling by at least about 20% to about 65%. In certain embodiments, the antibody described herein can attenuate (e.g., partially attenuate) IL-36 receptor mediated signaling by at least about 30% to about 65%.

Yet another non-limiting example of an IL-36α and/or IL-36γ activity is related to levels of cytokines and/or chemokines that are induced by IL-36. In some embodiments, the one or more cytokines and/or chemokines are selected from a group consisting of IL-8, IL-6, IL-10, TNFα, IL-1β, CXCL1, CCL5, CCL20, CCL2, CCL3, CCL4, CXCL12, VEGF-A, IL-23, IL-36α, IL-36β, and IL-36γ.

In one embodiment, the IL-36α and/or IL-36γ activity is related to IL-8 secretion. Thus, in certain embodiments, provided herein is a method of inhibiting IL-8 secretion in a cell, comprising exposing the cell to an effective amount of an antibody or antigen binding fragment thereof provided herein.

In one embodiment, an antibody provided herein inhibits IL-8 secretion by at least about 5%. In one embodiment, an antibody provided herein inhibits IL-8 secretion by at least about 10%. In one embodiment, an antibody provided herein inhibits IL-8 secretion by at least about 15%. In one embodiment, an antibody provided herein inhibits IL-8 secretion by at least about 20%. In one embodiment, an antibody provided herein inhibits IL-8 secretion by at least about 25%. In one embodiment, an antibody provided herein inhibits IL-8 secretion by at least about 30%. In one embodiment, an antibody provided herein inhibits IL-8 secretion by at least about 35%. In one embodiment, an antibody provided herein inhibits IL-8 secretion by at least about 40%. In one embodiment, an antibody provided herein inhibits IL-8 secretion by at least about 45%. In one embodiment, an antibody provided herein inhibits IL-8 secretion by at least about 50%. In one embodiment, an antibody provided herein inhibits IL-8 secretion by at least about 55%. In one embodiment, an antibody provided herein inhibits IL-8 secretion by at least about 60%. In one embodiment, an antibody provided herein inhibits IL-8 secretion by at least about 65%. In one embodiment, an antibody provided herein inhibits IL-8 secretion by at least about 70%. In one embodiment, an antibody provided herein inhibits IL-8 secretion by at least about 75%. In one embodiment, an antibody provided herein inhibits IL-8 secretion by at least about 80%. In one embodiment, an antibody provided herein inhibits IL-8 secretion by at least about 85%. In one embodiment, an antibody provided herein inhibits IL-8 secretion by at least about 90%. In one embodiment, an antibody provided herein inhibits IL-8 secretion by at least about 95%. In one embodiment, an antibody provided herein inhibits IL-8 secretion by at least about 96%. In one embodiment, an antibody provided herein inhibits IL-8 secretion by at least about 97%. In one embodiment, an antibody provided herein inhibits IL-8 secretion by at least about 98%. In one embodiment, an antibody provided herein inhibits IL-8 secretion by at least about 99%.

In one embodiment, the antibody provided herein inhibits IL-8 secretion with an 10o of at most about 100 nM. In one embodiment, the antibody provided herein inhibits IL-8 secretion with an 10o of at most about 90 nM. In one embodiment, the antibody provided herein inhibits IL-8 secretion with an 10o of at most about 80 nM. In one embodiment, the antibody provided herein inhibits IL-8 secretion with an 10o of at most about 70 nM. In one embodiment, the antibody provided herein inhibits IL-8 secretion with an 10o of at most about 60 nM. In one embodiment, the antibody provided herein inhibits IL-8 secretion with an IC₅₀ of at most about 50 nM. In one embodiment, the antibody provided herein inhibits IL-8 secretion with an IC₅₀ of at most about 40 nM. In one embodiment, the antibody provided herein inhibits IL-8 secretion with an IC₅₀ of at most about 30 nM. In one embodiment, the antibody provided herein inhibits IL-8 secretion with an IC₅₀ of at most about 20 nM. In one embodiment, the antibody provided herein inhibits IL-8 secretion with an IC₅₀ of at most about 10 nM. In one embodiment, the antibody provided herein inhibits IL-8 secretion with an IC₅₀ of at most about 1 nM. In one embodiment, the antibody provided herein inhibits IL-8 secretion with an IC₅₀ of at most about 0.1 nM. In one embodiment, the antibody provided herein inhibits IL-8 secretion with an IC₅₀ of at most about 0.05 nM. In one embodiment, the antibody provided herein inhibits IL-8 secretion with an IC₅₀ of at most about 0.001 nM.

In one embodiment, the antibody provided herein inhibits IL-8 secretion with an IC50 of at least about 100 nM. In one embodiment, the antibody provided herein inhibits IL-8 secretion with an IC50 of at least about 90 nM. In one embodiment, the antibody provided herein inhibits IL-8 secretion with an IC50 of at least about 80 nM. In one embodiment, the antibody provided herein inhibits IL-8 secretion with an IC50 of at least about 70 nM. In one embodiment, the antibody provided herein inhibits IL-8 secretion with an IC50 of at least about 60 nM. In one embodiment, the antibody provided herein inhibits IL-8 secretion with an IC50 of at least about 50 nM. In one embodiment, the antibody provided herein inhibits IL-8 secretion with an IC50 of at least about 40 nM. In one embodiment, the antibody provided herein inhibits IL-8 secretion with an IC50 of at least about 30 nM. In one embodiment, the antibody provided herein inhibits IL-8 secretion with an IC50 of at least about 20 nM. In one embodiment, the antibody provided herein inhibits IL-8 secretion with an IC50 of at least about 10 nM. In one embodiment, the antibody provided herein inhibits IL-8 secretion with an IC50 of at least about 1 nM. In one embodiment, the antibody provided herein inhibits IL-8 secretion with an IC50 of at least about 0.1 nM. In one embodiment, the antibody provided herein inhibits IL-8 secretion with an IC50 of at least about 0.05 nM. In one embodiment, the antibody provided herein inhibits IL-8 secretion with an IC50 of at least about 0.001 nM. In specific embodiments, the IC₅₀ is assessed by methods described herein, for example, in Section 6 below. In other embodiments, the IC₅₀ is assessed by other methods known to one of skill in the art.

Yet another non-limiting example of an IL-36α and/or IL-36γ activity is related to IL-36 receptor dimerization (i.e., heterodimerization between IL-36R and IL-1RAcP). Thus, in certain embodiments, provided herein is a method of attenuating IL-36 receptor dimerization in a cell, comprising exposing the cell to an effective amount of an antibody or antigen binding fragment thereof provided herein.

In some embodiments, the antibody provided herein attenuates IL-36 receptor dimerization by at least about 10%. In some embodiments, the antibody provided herein attenuates IL-36 receptor dimerization by at least about 15%. In some embodiments, the antibody provided herein attenuates IL-36 receptor dimerization by at least about 20%. In some embodiments, the antibody provided herein attenuates IL-36 receptor dimerization by at least about 25%. In some embodiments, the antibody provided herein attenuates IL-36 receptor dimerization by at least about 30%. In some embodiments, the antibody provided herein attenuates IL-36 receptor dimerization by at least about 35%. In some embodiments, the antibody provided herein attenuates IL-36 receptor dimerization by at least about 40%. In some embodiments, the antibody provided herein attenuates IL-36 receptor dimerization by at least about 45%. In some embodiments, the antibody provided herein attenuates IL-36 receptor dimerization by at least about 50%. In some embodiments, the antibody provided herein attenuates IL-36 receptor dimerization by at least about 55%. In some embodiments, the antibody provided herein attenuates IL-36 receptor dimerization by at least about 60%. In some embodiments, the antibody provided herein attenuates IL-36 receptor dimerization by at least about 65%. In some embodiments, the antibody provided herein attenuates IL-36 receptor dimerization by at least about 70%. In some embodiments, the antibody provided herein attenuates IL-36 receptor dimerization by at least about 75%. In some embodiments, the antibody provided herein attenuates IL-36 receptor dimerization by at least about 80%. In some embodiments, the antibody provided herein attenuates IL-36 receptor dimerization by at least about 85%. In some embodiments, the antibody provided herein attenuates IL-36 receptor dimerization by at least about 90%. In some embodiments, the antibody provided herein attenuates IL-36 receptor dimerization by at least about 95%.

Yet another non-limiting example of an IL-36α and/or IL-36γ activity is related to activation of mitogen-activated protein kinase (MAPK) pathways and/or nuclear factor kappa B (NF-κB) dependent transcription. Thus, in certain embodiments, provided herein is a method of attenuating activation of MAPK pathways and/or NF-κB dependent transcription in a cell, comprising exposing the cell to an effective amount of an antibody or antigen binding fragment thereof provided herein.

In certain embodiments, the antibody provided herein attenuates activation of MAPK pathways and/or NF-κB dependent transcription by at least about 10%. In certain embodiments, the antibody provided herein attenuates activation of MAPK pathways and/or NF-κB dependent transcription by at least about 15%. In certain embodiments, the antibody provided herein attenuates activation of MAPK pathways and/or NF-κB dependent transcription by at least about 20%. In certain embodiments, the antibody provided herein attenuates activation of MAPK pathways and/or NF-κB dependent transcription by at least about 25%. In certain embodiments, the antibody provided herein attenuates activation of MAPK pathways and/or NF-κB dependent transcription by at least about 30%. In certain embodiments, the antibody provided herein attenuates activation of MAPK pathways and/or NF-κB dependent transcription by at least about 35%. In certain embodiments, the antibody provided herein attenuates activation of MAPK pathways and/or NF-κB dependent transcription by at least about 40%. In certain embodiments, the antibody provided herein attenuates activation of MAPK pathways and/or NF-κB dependent transcription by at least about 45%. In certain embodiments, the antibody provided herein attenuates activation of MAPK pathways and/or NF-κB dependent transcription by at least about 50%. In certain embodiments, the antibody provided herein attenuates activation of MAPK pathways and/or NF-κB dependent transcription by at least about 60%. In certain embodiments, the antibody provided herein attenuates activation of MAPK pathways and/or NF-κB dependent transcription by at least about 65%. In certain embodiments, the antibody provided herein attenuates activation of MAPK pathways and/or NF-κB dependent transcription by at least about 70%. In certain embodiments, the antibody provided herein attenuates activation of MAPK pathways and/or NF-κB dependent transcription by at least about 75%. In certain embodiments, the antibody provided herein attenuates activation of MAPK pathways and/or NF-κB dependent transcription by at least about 80%. In certain embodiments, the antibody provided herein attenuates activation of MAPK pathways and/or NF-κB dependent transcription by at least about 85%. In certain embodiments, the antibody provided herein attenuates activation of MAPK pathways and/or NF-κB dependent transcription by at least about 90%. In certain embodiments, the antibody provided herein attenuates activation of MAPK pathways and/or NF-κB dependent transcription by at least about 95%.

In another aspect, provided herein is a method of treating a disease or disorder in a subject comprising comprising administering to the subject an effective amount of an antibody or antigen binding fragment thereof provided herein. In one embodiment, the disease or disorder is an IL-36-mediated disease or disorder. In one embodiment, the disease or disorder is an IL-36 receptor-mediated disease or disorder. In some embodiments, the disease or disorder is related to skin, intestinal and/or lung tissues. Also provided herein is a method of treatment of a disease or disorder, wherein the subject is administered one or more therapeutic agents in combination with the antibody or antigen-binding fragment thereof provided herein. Methods of administration and dosing is described in more detail in Section 5.7 below.

In another aspect, provided herein is the use of the antibody or antigen binding fragment thereof provided herein in the manufacture of a medicament for treating a disease or disorder in a subject.

In another aspect, provided herein is the use of a pharmaceutical composition provided herein in the manufacture of a medicament for treating a disease or disorder in a subject.

In another aspect, provided herein is the use of an antibody or antigen binding fragment thereof provided herein in the manufacture of a medicament, wherein the medicament is for use in a method for detecting the presence of an IL-36 protein (e.g., an IL-36α and/or IL-36γ) in a biological sample, the method comprising contacting the biological sample with the antibody under conditions permissive for binding of the antibody to the IL-36 protein, and detecting whether a complex is formed between the antibody and the IL-36 protein.

In other aspects, the antibodies and fragments thereof of the present disclosure are useful for detecting the presence of an IL-36 protein (e.g., an IL-36α and/or IL-36γ) in a biological sample. The term “detecting” as used herein encompasses quantitative or qualitative detection. In certain embodiments, a biological sample comprises bodily fluid, a cell, or a tissue. Diagnostic assays and methods are described in more detail in Section 5.9 below.

### 5.7 Methods of Administration and Dosing

In a specific embodiment, provided herein is a composition for use in the prevention and/or treatment of a disease or condition comprising an antibody or antigen binding fragment thereof provided herein. In one embodiment, provided herein is a composition for use in the prevention of a disease or condition, wherein the composition comprises an antibody or antigen binding fragment thereof provided herein. In one embodiment, provided herein is a composition for use in the treatment of a disease or condition, wherein the composition comprises an antibody or antigen binding fragment thereof provided herein. In some embodiments, the disease or condition is an IL-36-mediated disease. In some embodiments, the disease or condition is an IL-36-mediated disease. In some embodiments, the disease or condition is related to skin, intestinal and/or lung tissues. In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided herein. In one embodiment, provided herein is a composition for use in the prevention of a symptom of a disease or condition, wherein the composition comprises an antibody or antigen binding fragment thereof provided herein. In one embodiment, provided herein is a composition for use in the treatment of a symptom of a disease or condition, wherein the composition comprises an antibody or antigen binding fragment thereof provided herein. In some embodiments, the disease or condition is an IL-36-mediated disease. In one embodiment, the disease is related to skin, intestinal and/or lung tissues. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein.

In some embodiments, the disease or disorder is a disease or disorder mediated by IL-36α and/or IL-36γ. In some embodiments, the disease or disorder is an inflammatory autoimmune disease or disorder. In some embodiments, the inflammatory autoimmune disease or disorder is related to skin tissue, intestinal tissue and/or lung tissue. In some embodiments, the disease or disorder is selected from a group consisting of generalized pustular psoriasis, palmoplantar pustulosis, palmoplantar pustular psoriasis, discoid lupus erythematosus, lupus erythematosus, atopic dermatitis, Crohn's disease, ulcerative colitis, asthma, inflammatory bowel diseases, psoriasis vulgaris, acrodermatitis continua of Hallopeau, acute generalized exanthematous pustulosis, hidradenitis suppurativa, lichen planus, Sjögren's syndrome, rheumatoid arthritis, psoriatic arthritis, chronic rhinosinusitis, acne vulgaris, impetigo herpetiformis, pyoderma gangrenosum, and polymorphic light eruption. In some embodiments, the disease or condition is an IL-36 receptor mediated disease. In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In some embodiments, the disease or condition is an IL-36-mediated disease. In one embodiment, the disease or condition is related to skin, intestinal and/or lung tissues. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

WO 2020/065594 PCT/IB2019/058203

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. In one aspect, the antibody or antigen binding fragment thereof is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus monkey, or a human). In a one embodiment, the subject is a human. In another embodiment, the subject is a human with a disease or condition.

Various delivery systems are known and can be used to administer a prophylactic or therapeutic agent (e.g., an antibody or antigen binding fragment thereof provided herein), including, but not limited to, encapsulation in liposomes, microparticles, microcapsules, recombinant cells capable of expressing the antibody or antigen binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody or antigen binding fragment thereof provided herein, care must be taken to use materials to which the antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et aL, in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek etaL, 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Patent No. 5,679,377; U.S. Patent No. 5,916,597; U.S. Pat. Nos. 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibody or antigen binding fragment thereof provided herein. See, e.g., U.S. Patent No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more antibody or antigen binding fragment thereofs provided herein. In another embodiment, a composition provided herein comprises one, two or more antibody or antigen binding fragment thereofs provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, Pa. Such compositions will contain a prophylactically or therapeutically effective amount of the antibody or antigen binding fragment thereof provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In one embodiment, the antibody or antigen binding fragment thereof is supplied as a dry sterilized lyophilized powder or water free concentrate in a hermetically sealed container and can be reconstituted, e.g., with water or saline to the appropriate concentration for administration to a subject. The lyophilized antibody or antigen binding fragment thereof can be stored at between 2 and 8° C. in its original container and the antibody or antigen binding fragment thereof can be administered within 12 hours, such as within 6 hours, within 5 hours, within 3 hours, or within 1 hour after being reconstituted. In an alternative embodiment, an antibody or antigen binding fragment thereof provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody or antigen binding fragment thereof provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody or antigen binding fragment thereof provided herein to a patient is intranasal, intramuscular, intravenous, or a combination thereof, but other routes described herein are also acceptable. Each dose may or may not be administered by an identical route of administration. In some embodiments, an antibody or antigen binding fragment thereof provided herein may be administered via multiple routes of administration simultaneously or subsequently to other doses of the same or a different antibody or antigen binding fragment thereof provided herein.

In certain embodiments, the antibody or antigen binding fragment thereofs provided herein are administered prophylactically or therapeutically to a subject. The antibody or antigen binding fragment thereofs provided herein can be prophylactically or therapeutically administered to a subject so as to prevent, lessen or ameliorate a disease or symptom thereof.

### 5.8 Gene Therapy

In a specific embodiment, nucleic acids comprising sequences encoding antibodies or functional derivatives thereof, are administered to a subject for use in a method provided herein, for example, to prevent, manage, treat and/or ameliorate an IL-36-mediated disease, disorder or condition, by way of gene therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932 ; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding an antibody provided herein, the nucleic acids being part of an expression vector that expresses the antibody or chimeric proteins or heavy or light chains thereof in a suitable host. In particular, such nucleic acids have promoters, such as heterologous promoters, operably linked to the antibody coding region, the promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In some embodiments, the expressed antibody molecule is a single chain antibody; alternatively, the nucleic acid sequences include sequences encoding both the heavy and light chains, or fragments thereof, of the antibody.

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; W093/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an anti-IL-36 antibody as provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.9 Diagnostic Assays and Methods

Labeled antibodies and derivatives and analogs thereof, which immunospecifically bind to an IL-36 antigen (e.g., an IL-36α and/or IL-36γ antigen) can be used for diagnostic purposes to detect, diagnose, or monitor an IL-36-mediated disease. Thus, provided herein are methods for the detection of an IL-36-mediated disease comprising: (a) assaying the expression of an IL-36 antigen in cells or a tissue sample of a subject using one or more antibodies provided herein that immunospecifically bind to the IL-36 antigen; and (b) comparing the level of the IL-36 antigen with a control level, e.g., levels in normal tissue samples (e.g., from a patient not having an IL-36-mediated disease, or from the same patient before disease onset), whereby an increase in the assayed level of IL-36 antigen compared to the control level of the IL-36 antigen is indicative of an IL-36-mediated disease.

Also provided herein is a diagnostic assay for diagnosing an IL-36-mediated disease comprising: (a) assaying for the level of an IL-36 antigen in cells or a tissue sample of an individual using one or more antibodies provided herein that immunospecifically bind to an IL-36 antigen; and (b) comparing the level of the IL-36 antigen with a control level, e.g., levels in normal tissue samples, whereby an increase in the assayed IL-36 antigen level compared to the control level of the IL-36 antigen is indicative of an IL-36-mediated disease. In certain embodiments, provided herein is a method of treating an IL-36-mediated disease in a subject, comprising: (a) assaying for the level of an IL-36 antigen in cells or a tissue sample of the subject using one or more antibodies provided herein that immunospecifically bind to an IL-36 antigen; and (b) comparing the level of the IL-36 antigen with a control level, e.g., levels in normal tissue samples, whereby an increase in the assayed IL-36 antigen level compared to the control level of the IL-36 antigen is indicative of an IL-36-mediated disease. In some embodiments, the method further comprises (c) administering an effective amount of an antibody provided herein to the subject identified as having the IL-36-mediated disease. A more definitive diagnosis of an IL-36-mediated disease may allow health professionals to employ preventative measures or aggressive treatment earlier thereby preventing the development or further progression of the IL-36-mediated disease.

Antibodies provided herein can be used to assay IL-36 antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell . Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (121In), and technetium (99Tc); luminescent labels, such as luminol; and fluorescent labels, such as fluorescein and rhodamine, and biotin.

One aspect provided herein is the detection and diagnosis of an IL-36-mediated disease in a human. In one embodiment, diagnosis comprises: a) administering (for example, parenterally, subcutaneously, or intraperitoneally) to a subject an effective amount of a labeled antibody that immunospecifically binds to an IL-36 antigen; b) waiting for a time interval following the administering for permitting the labeled antibody to concentrate at sites in the subject where the IL-36 antigen is expressed (and for unbound labeled molecule to be cleared to background level); c) determining background level; and d) detecting the labeled antibody in the subject, such that detection of labeled antibody above the background level indicates that the subject has an IL-36-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S.W. Burchiel and B. A Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of an IL-36-mediated disease is carried out by repeating the method for diagnosing the an IL-36-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.10 Kits

Also provided herein are kits comprising an antibody (e.g., an anti-IL-36 antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to an IL-36 antigen. In specific embodiments, provided herein are panels of antibodies having different association rate constants different dissociation rate constants, different affinities for IL-36 antigen (e.g., IL-36α and/or IL-36γ), and/or different specificities for an IL-36 antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254...500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In an amino acid sequence, each amino acid residue can be identified by the position of the amino acid residue in the sequence and the type of amino acid. For example, assuming the first amino acid residue in a sequence is glycine, this glycine can be represented as 1st Gly, Gly 1 or G1. For another example, the language “45th Arg of the amino acid sequence of IL-36α represented by SEQ ID NO: 5 or SEQ ID NO: 7” refers to the 45th amino acid residue in SEQ ID NO: 5 or SEQ ID NO: 7, which is Arg.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 6.1 Example 1—Generation of Mouse Anti-Human IL-36α and IL-36γ Dual Antagonist Antibodies with Cross-Reacativity to Cynomolgus Macaque IL-36α and IL-36γ

This example illustrates the method for generating exemplary mouse anti-human IL-36α and IL-36γ dual-antagonist antibodies provided herein. It is to be understood that the exemplary anti-IL-36 antibodies described in this example are not intended to represent the full scope of the present invention.

Prokaryotic expression vectors for producing recombinant human and cynomolgus macaque IL-36 proteins were fabricated so as to produce high-activity, “truncated” IL-36 proteins, fused at the amino terminus with a His-SUMO tag.

Specifically, DNA sequences encoding human IL-36α(R12), a protein variant with glutamine-to-arginine substitution at position 12 (Q12R) (dbSNP:rs895497 of GenBank™ accession no. NM_014440), and human IL-36β (GenBank™ accession no. NM_173178) were purchased (cat #RC219328 and RC211037, respectively, Origene, Rockville, Md.). The DNA encoding human IL-36α(R12) has a polynucleotide sequence of SEQ ID NO: 4, and the encoded human IL-36α(R12) has an amino acid sequence of SEQ ID NO: 5. The DNA encoding truncated human IL-36β has a polynucleotide sequence of SEQ ID NO: 8, and the encoded truncated human IL-36β has an amino acid sequence of SEQ ID NO: 9.

Prokaryotic expression vectors for amino-terminus-tagged, truncated IL-36 cytokines were generated using the Expresso® T7 SUMO Cloning and Expression System (cat #49013-1, Lucigen, Middleton, Wis.), as per manufacturer's manual. Briefly, the sequence coding for the highly active “truncated” form of human IL-36α(R12) (amino acids Lys6-Phe158) (nucleotide sequence of SEQ ID NO: 4, amino acid sequence of SEQ ID NO: 5) or truncated human IL-36β (amino acids Arg5-Glu157) (nucleotide sequence of SEQ ID NO: 8, amino acid sequence of SEQ ID NO: 9) were amplified by PCR and ligated into the pETite prokaryotic expression vector (cat # 49013-1, Lucigen, Middleton, Wis.), in frame with DNA coding for an amino terminal 6×Histidine tag followed by a SUMO protein tag. The polynucleotide sequence of the SUMO protein tag is as follows:

(SEQ ID NO: 18)
ATGCATCATCACCACCATCACGGGTCCCTGCAGGA
CTCAGAAGTCAATCAAGAAGCTAAGCCAGAGGTCA
AGCCAGAAGTCAAGCCTGAGACTCACATCAATTTA
AAGGTGTCCGATGGATCTTCAGAGATCTTCTTCAA
GATCAAAAAGACCACTCCTTTAAGAAGGCTGATGG
AAGCGTTCGCTAAAAGACAGGGTAAGGAAATGGAC
TCCTTAACGTTCTTGTACGACGGTATTGAAATTCA
AGCTGATCAGACCCCTGAAGATTTGGACATGGAGG
ATAACGATATTATTGAGGCTCACCGCGAACAGATT
GGAGGT.

The amino acid sequence of the SUMO protein tag is as follows:

(SEQ ID NO: 19)
MHHHHHHGSLQDSEVNQEAKPEVKPEVKPETHINL
KVSDGSSEIFFKIKKTTPLRRLMEAFAKRQGKEMD
SLTFLYDGIEIQADQTPEDLDMEDNDIMAHREQIG
G.

The expression vector for truncated human IL-36α(Q12) (GenBank™ accession no. NM_014440) (amino acids Lys6-Phe158) (nucleotide sequence of SEQ ID NO: 6, amino acid sequence of SEQ ID NO: 7) was generated the same way as IL-36α(R12), but by using a forward PCR primer that contained the nucleotide sequence for a glutamine-12 (Q12) codon instead of arginine-12 (R12) codon. Completed vector sequences were confirmed by Sanger sequencing. The same process was performed for generation of the homologous truncated macaque (Macaca fascicularis) IL-36 expression vectors. DNAs coding for truncated cynomolgus IL-36α (amino acids Lys6-Phe158) (accession no. XP_015288898.1) (nucleotide sequence of SEQ ID NO: 12, amino acid sequence of SEQ ID NO: 13), truncated cynomolgus IL-36β (amino acids Trp5-Glu157) (accession no. XP_005575353) (nucleotide sequence of SEQ ID NO: 14, amino acid sequence of SEQ ID NO: 15), and truncated cynomolgus IL-36γ (amino acids Ser18-Lys168) (accession no. XP 015288884) (nucleotide sequence of SEQ ID NO: 16, amino acid sequence of SEQ ID NO: 17) were synthesized (Thermo Fisher Scientific GeneArt, Regensberg, Germany) and used as PCR template for amplification and vector cloning. All completed vector sequences were validated by Sanger sequencing.

Recombinant truncated human IL-36γ (amino acids Ser18-Asp169) (GenBank™ accession no. NP_062564.1) (SEQ ID NO: 10), and recombinant truncated human IL-36Rα (amino acids Va12-Asp155) (GenBank™ accession no. NP_036407, UniProt accession no. Q9UBH0) (SEQ ID NO: 11) proteins were purchased from R&D Systems (Minneapolis, Minn.) (cat #s 6835-IL/CF and 1275-IL-025/CF, respectively).

Recombinant His-SUMO-tagged, truncated IL-36 cytokine proteins were produced in E. coli. To do this, pETite prokaryotic expression vectors containing DNA coding for His-SUMO-tagged, truncated human or cynomolgus IL-36 proteins (fabrication described above) were introduced into HI-Control BL21(DE3) bacterial cells (cat # 60110, Lucigen, Middleton, Wis.). A single kanamycin-resistant bacterial colony of each was grown in lysogeny-broth (LB) liquid culture containing kanamycin, 37° C. shaking 250 rpm, and induced to produce protein by addition of IPTG (Isopropyl β-D-thiogalactopyranoside) (cat #C0012, BioPioneer, San Diego, Calif.) when cell density, measured by OD600, reached ˜0.5-1. After 4-6 hours, cells were harvested by centrifugation and processed for protein purification.

His-tagged SUMO-IL36 alpha or beta recombinant proteins were expressed in E. coli and purified as briefly described below. Bacterial pellets containing induced protein of interest were re-suspended on ice in 20 mM Tris-HCl pH 8.0, 0.5 M NaCl, 10% glycerol Lysis Buffer supplemented with Protease Inhibitor Cocktail EDTA free tablets (cat# 5056489001, Sigma Aldrich, St. Louis, Mo.), followed by sonication on ice at 60% Amp for 15 sec, 70% Amp for 15 sec, and 80% Amp 3×15 sec. Sample was clarified by centrifugation at 20000×g, 4° C. for 30 min. Collected supernatants were diluted with 20 mM Tris-HCl pH 8.0, 0.5 M NaCl buffer and filtered with a 0.22 μm vacuum filter unit (Millipore, Bedford, Mass.), followed by purification using immobilized metal ion affinity chromatography (IMAC) (HisTrap HP cat#17524701, GE Healthcare Life Sciences, Pittsburgh, Pa.). 20 mM imidazole and 1 mM DTT (final concentration) were added just prior to purification. Sample was loaded onto a 5 mL HisTrap HP column equilibrated with 20 mM Tris-HCl pH 8.0, 0.5 M NaCl, 20 mM imidazole. Upon sample loading, the column was washed thoroughly with 6 column volumes (CV) of 20 mM Tris-HCl pH 8.0, 0.5 M NaCl, 20 mM imidazole. The protein was eluted with 20 mM-600 mM imidazole gradient over 25 CV, and neutralized with 5 mM EDTA and 5 mM DTT (final concentration). Fractions were analyzed by SDS-PAGE and positive fractions were pooled and dialyzed against PBS pH 7.4 (cat# P3813, Sigma Aldrich, St. Louis, Mo.). Following dialysis, the protein sample was concentrated with a centrifugal filter concentrator (Vivaspin 30,000 MWCO Cat#VS2022, Sartorius, Goettingen, Germany). Finally, the protein was filter sterilized using syringe filters with 0.22 μm pore diameter and protein concentration was determined using the Lowry method.

Cleaved, untagged human IL-36 proteins were obtained by enzymatic treatment of His-SUMO-IL36 with SUMO Express protease, according to the manufacturer protocol (cat# 30801-2, Lucigen, Middleton, Wis.). After the cleavage, the mixture was applied to an IMAC column, and the free target protein was recovered in the flow-through; the His-SUMO tag and SUMO Express Protease remained bound to the IMAC matrix. The fractions were analyzed by SDS-PAGE and the positive fractions were pooled and dialyzed against PBS pH 7.4 (cat# P3813, Sigma Aldrich, St. Louis, Mo.). Pyrogen content was determined using FDA-licensed Endosafe-PTS Limulus Amebocyte Lysate (LAL) assay (Charles River Laboratories, San Diego, Calif.). The limits of detection for this assay are 1-0.01 EU/mL of endotoxin. If the test was negative, the samples were considered endotoxin free.

After cleavage and removal of the His-SUMO tag from each truncated IL-36 protein, a highly pure, untagged, high-activity truncated form of each respective IL-36 cytokine protein remained: human IL-36α(R12) (SEQ ID NO: 5), human IL-36α (Q12) (SEQ ID NO: 7), human IL-36β (SEQ ID NO: 9), cynomolgus IL-36α (SEQ ID NO:13), cynomolgus (SEQ ID NO: 15), and cynomolgus IL-36γ (SEQ ID NO: 17). Unless otherwise noted, assays involving human IL-36α utilized IL-36α(R12).

CD2F1 mice purchased from Charles River and maintained at La Jolla Institute animal facility under pathogen free condition were immunized with recombinant human IL-36α, IL-36β, or IL-36γ. Several immunization strategies were applied. 1.5 to 10 μg of recombinant IL-36α, IL-36β, and IL-36γ were utilized as immunogens and administered to the animals individually, as a mixture, or sequentially. Adjuvants utilized include TiterMax Gold (Cat# 2684, Sigma, St. Louis, Mo.) for first immunizations, and aluminum hydroxide gel (Cat# vac-alu-250, InvivoGen, San Diego, Calif.) and toll-like receptor agonist CpG (Cat# InvivoGen, San Diego, Calif.) for subsequent boosts. Initial immunizations and boosts were performed subcutaneously on a weekly or bi-weekly basis. CD40 acts as a costimulatory molecule for the activation of B cells and in some instances an agonist antibody against mouse CD40 (Cat# BP0016-2, BioXCell, West Lebanon, N.H.) was administered via IP injection of 100 μg per mouse seven days after the first immunization.

Hybridomas were generated from mice immunized with recombinant human IL-36α, IL-36β, or IL-36γ. When a significant antigen specific serum titer was achieved, mice were sacrificed three to four days after the last boost. Draining lymph nodes and spleens were harvested and homogenized to make a single cell suspension. To generate hybridomas, cells were fused with myeloma cells Sp2/0-Ag14 (Cat# CRL-1581, ATCC, Manassas, Va.) at variable ratios ranging from 2:1 to 5:1. Fusion was induced via polyethylene glycol (Cat# 10783641001, Sigma, St. Louis, Mo.) or electrofusion (Cat# 450012, Harvard Apparatus, Holliston, Mass.). Successfully fused hybridoma cells were selected for by culture in HAT containing media (Cat# H0262, Sigma, St. Louis, Mo.).

In some instances, fused cells were seeded into 96-well plates at a density of 5,000 cells per well and screened one week later for binding to human and cynomolgus IL-36α, IL-36β, or IL-36γ using ELISA binding assays. In other instances, fused cells would be seeded into HAT containing semi-solid media (Cat# 03803, StemCell Technologies, Tukwila, Wash.) and monoclonal hybridomas were picked one week later with a ClonePix2 Instrument (Molecular Devices, Sunnyvale, Calif.). Picked hybridomas were screened one week later for human and cynomolgus IL-36αa, IL-36β, or IL-36γ by ELISA binding assays. Hybridomas showing positive binding by ELISA were sub-cloned if necessary, and rescreened for binding. In IL-36 ELISA binding assays, 96-well ELISA plates (Cat# 07-200-37, Fisher Scientific, Hanover Park, Ill.) were coated with recombinant human or cynomolgus IL-36α, IL-36β, or IL-36γ protein and incubated overnight at 4° C. Plates were washed (PBS with 0.05% Tween20) and blocked with 2% bovine serum albumin in PBS for 1 hour at room temperature. Plates were washed and hybridoma supernatants were added to the plates and incubated for 1 hour at room temperature. Plates were washed and binding of antibodies to the coated IL-36 antigens was detected with goat anti-mouse IgG-HRP (Cat# 109-036-098, Jackson ImmunoResearch, West Grove, Pa.). Plates were then washed and TMB substrate (Cat# 1721067, Bio-Rad, Los Angeles, Calif.) was added. Following sufficient color development the reaction was stopped with H₂SO₄ and a microplate reader measured the optical density at 450 nM.

Hybridomas producing monoclonal antibodies with binding to IL-36 cytokines were screened for antagonist activity in a high-throughput screen assay utilizing HaCaT cells (Cat# T0020001, AddexBio, San Diego, Calif.), a human keratinocyte cell line.

HaCaT cells were cultured in DMEM (CAT# 10313-021, Life Technologies, Carlsbad, Calif.), 10% heat-inactivated FBS (Cat# SH30071.03, Thermo Fisher Scientific, Asheville, N.C.), and 1% PenStrep (Missouri Cat# P0781, Sigma, St. Louis). For high throughput screens 10 μL of hybridoma culture supernatant were added to 384-well assay plates (Cat# 3701, Fisher Scientific, Hanover Park, Illinois) followed by addition of 10 μL of human IL-36α, IL-36β, or IL-36γ cytokines at a concentration of 30 nM in HaCaT culture media. 10 μL of IL-36Ra (Cat# 1275-IL/CF, R&D Systems, Minneapolis, Minn.) at 3000 nM was added as a positive control for antagonism of IL-36 receptor signaling. 10 μL of HaCaT cells were added at a concentration of 1×10⁶ cells/ml. Final assay conditions contained 10,000 HaCaT cells per well, 10 nM of IL-36α, IL-36β, or IL-36γ cytokines, and 1000 nM IL-36Ra. Assay plates were incubated at 5% CO₂, 37° C. for 20 hours. Assay culture supernatant was then collected and secreted IL-8 was measured with the IL-8 Ready-SET-Go! ELISA Kit as per manufacturer's instructions (Cat# 88-8086-88, Life Technologies, Carlsbad, Calif.). To accommodate a 384-well screening format, ELISA kit reagents were used at 15 μL volumes. Assay culture supernatants were diluted 5-fold into the ELISA kit reagent diluent and 25 μL of the sample was evaluated in the IL-8 ELISA. A reduction in O.D. 450 values in the IL-8 ELISA was utilized to identify IL-36 antagonist monoclonal antibodies.

Monoclonal antibodies were purified from hybridomas exhibiting antagonist activity and evaluated for IC₅₀ potency in the HaCaT assay. For evaluation of the IC₅₀ potency of the IL-36 monoclonal antibodies in antagonizing human IL-36α, IL-36β, and IL-36γ, purified monoclonal antibodies were diluted into HaCaT culture media to a 600 nM concentration followed by a 2-fold dilution series. 10 μL of diluted antibodies were transferred to the assay plate for a final assay concentration starting at 200 nM, followed by a 2-fold dilution series. IL-36Ra was utilized as a positive control for IL-36 receptor antagonism and was handled in a similar fashion as the IL-36 monoclonal antibodies, though the final assay concentration often started at 1000 nM, followed by a 2-fold dilution series. O.D. 450 values from the IL-8 ELISA were graphed and IC₅₀ values were calculated using GraphPad PRISM™ software. For evaluation of the IC₅₀ potency of the IL-36 monoclonal antibodies in antagonizing cynomolgus IL-36α, IL-36β, and IL-36γ, a similar protocol was applied in which cynomolgus IL-36 cytokines were utilized in place of the human IL-36 cytokines.

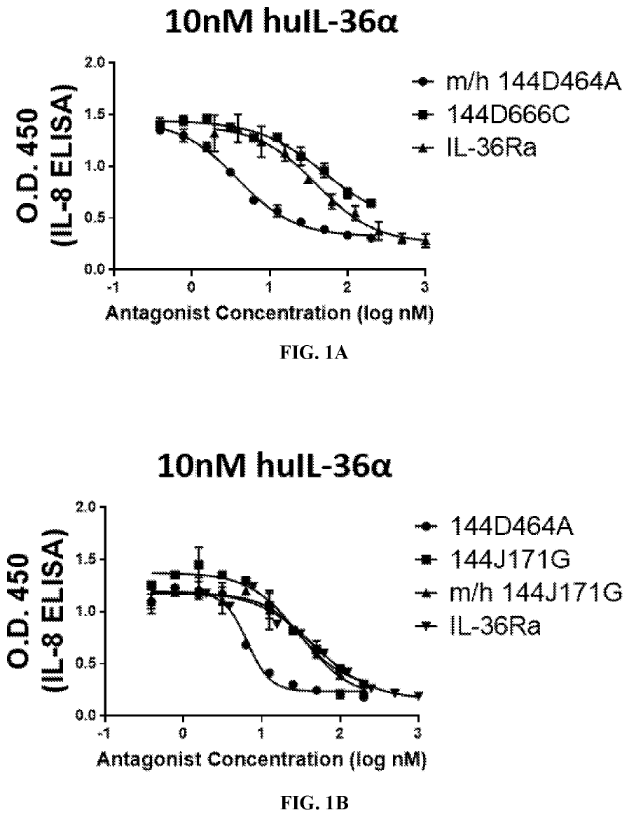

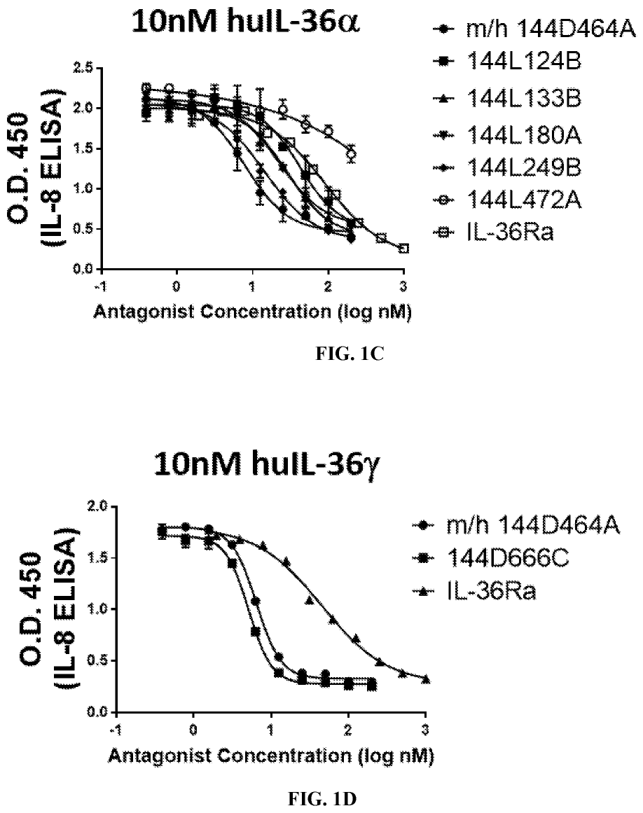

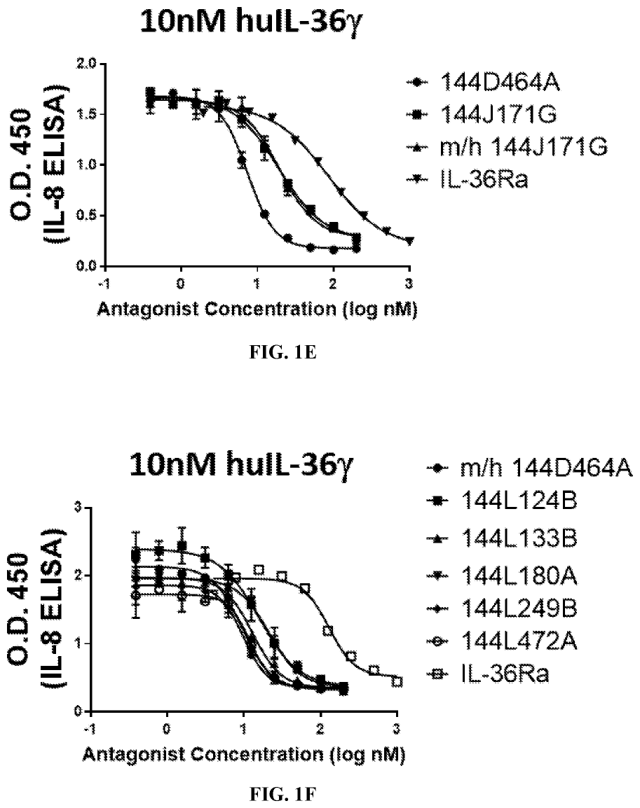

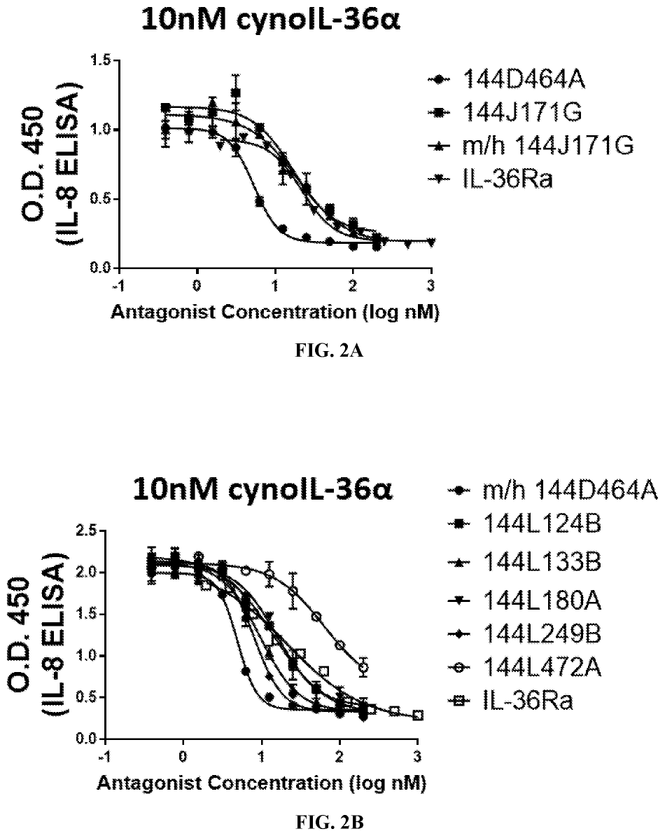

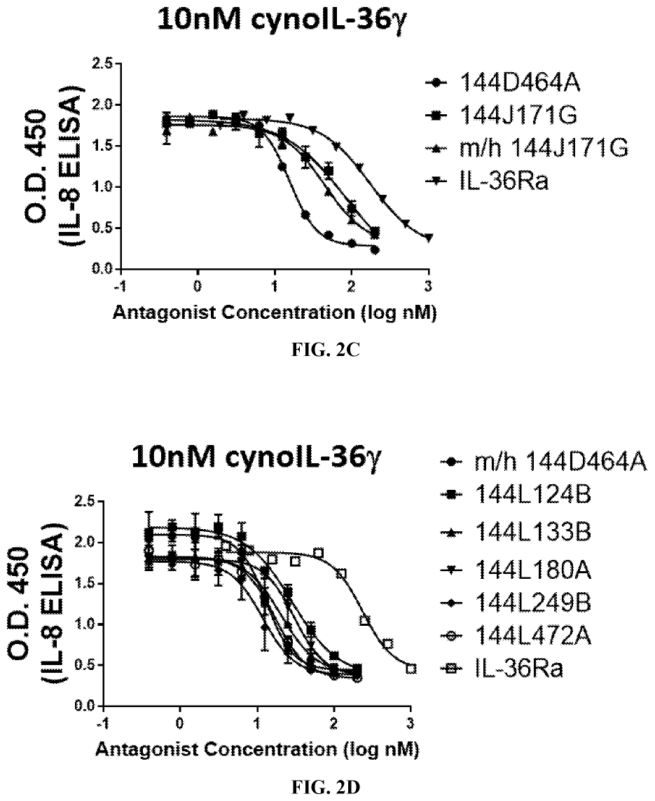

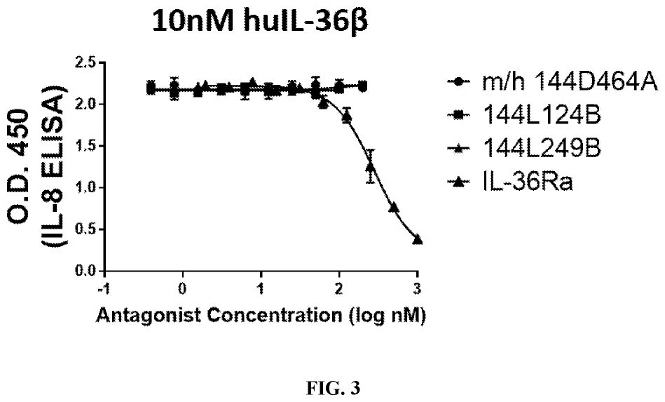

Eight monoclonal antibodies with binding and antagonist activity against human and cynomolgus IL-36α and IL-36γ were identified. Functional activity was maintained when selected monoclonal antibodies were converted to mouse/human (m/h) chimeric antibodies (see FIG. 1A to 1F and FIG. 2A to 2D). IC₅₀ values of antibodies and IL-36Ra antagonizing human and cynomolgus IL-36α (see Table 1 below) and IL-36γ (see Table 2 below) in HaCaT functional assays are summarized in Tables 1 and 2. These antibodies did not bind IL-36Ra nor do they bind or antagonize IL-36β (see FIG. 3). Many of the evaluated antibodies exhibited greater potency in antagonizing IL-36α or IL-36γ than IL-36Ra.

TABLE 1
IC₅₀ values of antibodies and IL-36Ra antagonizing human
and cynomolgus IL-36α in HaCaT functional assays

| | FIG. 1A | FIG. 1B | FIG. 1C | FIG. 2A | FIG. 2B |
|---|---|---|---|---|---|
| | 10 nM | 10 nM | 10 nM | 10 nM | 10 nM |
| IC₅₀ | human | human | human | cynomolgus | cynomolgus |
| (nM) | IL-36α | IL-36α | IL-36α | IL-36α | IL-36α |
| 144D464A | n.t | 6.493 | n.t | 5.305 | n.t |
| m/h | 3.838 | n.t | 7.767 | n.t | 4.907 |
| 144D464A | | | | | |
| 144D666C | 49.53 | n.t | n.t | n.t | n.t |
| 144J171G | n.t | 26.99 | n.t | 16.82 | n.t |
| m/h | n.t | 39.2 | n.t | 19.33 | n.t |
| 144J171G | | | | | |
| 144L124B | n.t | n.t | 42.03 | n.t | 14.23 |
| 144L133B | n.t | n.t | 28.68 | n.t | 9.605 |
| 144L180A | n.t | n.t | 22.52 | n.t | 15.69 |
| 144L249B | n.t | n.t | 14.53 | n.t | 7.937 |
| 144L472A | n.t | n.t | Weak | n.t | 63.19 |
| IL-36Ra | 39.91 | 46.59 | 90.63 | 21.83 | 20.83 |

Note:
n.t. means not tested

TABLE 2
IC₅₀ values of antibodies and IL-36Ra antagonizing human
and cynomolgus IL-36γ in HaCaT functional assays

| | FIG. 1D | FIG. 1E | FIG. 1F | FIG. 2C | FIG. 2D |
|---|---|---|---|---|---|
| | 10 nM | 10 nM | 10 nM | 10 nM | 10 nM |
| IC₅₀ | human | human | human | cynomolgus | cynomolgus |
| (nM) | IL-36γ | IL-36γ | IL-36γ | IL-36γ | IL-36γ |
| 144D464A | n.t | 7.386 | n.t | 15.58 | n.t |
| m/h | 6.432 | n.t | 9.536 | n.t | 13.15 |
| 144D464A | | | | | |
| 144D666C | 5.12 | n.t | n.t | n.t | n.t |
| 144J171G | n.t | 19.23 | n.t | 82.69 | n.t |
| m/h | n.t | 19.41 | n.t | 44.44 | n.t |
| 144J171G | | | | | |
| 144L124B | n.t | n.t | 15.83 | n.t | 28.29 |
| 144L133B | n.t | n.t | 12.94 | n.t | 20.55 |
| 144L180A | n.t | n.t | 21.31 | n.t | 30.34 |
| 144L249B | n.t | n.t | 9.806 | n.t | 11.8 |
| 144L472A | n.t | n.t | 11.95 | n.t | 17.07 |
| IL-36Ra | 45.09 | 86.47 | 128.9 | 174.3 | 234.9 |

Note:
n.t. means not tested

### 6.2 Example 2—Antibody Preparation

6.2.1 Cloning of Genes Encoding VH and VL of anti-IL-36 Antibody From Hybridoma Cells

Nucleotide sequences of antibody variable regions were determined by sequencing VH and VL genes isolated by 5′ RACE-PCR amplification from RNA extracted from clonal hybridoma cells. Total RNA was isolated from 1×10⁶ hybridoma cells producing 144D464A, 144D666C, 144J171G, 144L124B, 144L133B, 144L180A, 144L249B, or 144L472A, antibodies using an RNeasy Mini Kit (cat # 74104, QIAGEN, Hilden, Germany) and QIA shredder (cat # 79654, QIAGEN, Hilden, Germany). First strand cDNA was synthesized using 1 μg of total RNA for each hybridoma using a SMARTer® RACE cDNA Amplification Kit (cat # 634858, TaKaRa Bio USA, Mountain View, Calif.). cDNA sequence for each unique VH and VL was obtained using first strand cDNA as the template. PCR was performed using primers specific to mouse IgG (GATTACGCCAAGCTTGTCACTGGCTCAGGGAAATAA (SEQ ID NO: 97)), and the universal primer A (provided in SMARTer® RACE cDNA Amplification Kit), to amplify the VH cDNA fragment of each antibody. In addition, PCR was performed using primers specific to mouse IgLambda (GATTACGCCAAGCTTCTCYTCAGRGGAAGGTGGRAACA (SEQ ID NO: 98), in which “R” stands for either A or G, and “Y” stands for either C or T) and the universal primer A in order to amplify the VL cDNA fragment of each antibody. Subsequently, each PCR reaction was subjected to gel electrophoresis and amplified fragments were purified using a QIAquick Gel Extraction Kit (cat # 28704, QIAGEN, Hilden, Germany). Each of the gene fragments obtained was inserted into a linearized pRACE vector (supplied with SMARTer® RACE Kit) or a linearized pCR4 vector using a Zero Blunt TOPO PCR Cloning Kit for Sequencing (cat # K280020, Life Technologies, Carlsbad, Calif.).

The resulting plasmids containing amplified variable gene nucleotide sequences were introduced into competent E. coli, DH5a or TOP10 (cat # 18265017 or C404003, Life Technologies, Carlsbad, Calif.), and selected for using the appropriate antibiotic on LB-agar plates. DNA plasmids were subsequently amplified by growing individual bacterial clones in liquid LB culture, under antibiotic selection, and then isolated by extraction using QIAprep Spin Miniprep kit (cat #27104, QIAGEN, Hilden, Germany).

Complete sequence for the VH and VL of each antibody clone was determined by Sanger sequencing of the PCR-derived inserts from multiple plasmids for each clone, and aligning the results using Sequencher 5.4.6 (Gene Codes Corporation, Ann Arbor, Michigan). The consensus nucleotide sequence for each was determined to be the full-length VH or VL cDNA (including the putative ATG initiation codon at the 5′-terminus). The respective VH and VL amino acid sequences were deduced from these.

The nucleotide and amino acid sequences of the VH and VL regions (with or without signal sequences) of the identified eight antibodies are listed in the tables below.

TABLE 3
VH nucleic acid sequences,
including the signal sequences

| | Nucleotide sequences |
|---|---|
| Antibody | (SEQ ID NO:) |
| 144D464A | ATGAAATGCAGCTGGGTTATCTT |
| | CTTCCTGATGGCAGTGGTTACAG |
| | GGGTCAATTCTGAGGTTCAGCTG |
| | CAGCAGTCTGGGGCAGAGCTTGT |
| | GAAGCCAGGGGCCTCAGTCAGGT |
| | TGTCCTGCACAGCTTCTGGCTTC |
| | AACATTAAAGACACCTATATACA |
| | CTGGGTGAAGCAGAGGCCTGAAC |
| | AGGGCCTGGAGTGGATTGGAAGG |
| | ATTGATCCTGCGATTGGTACTAC |
| | TAGATATGACCCGAAGTTCCAGG |
| | GCAAGGCCACTATAACAACAGAC |
| | ACATCCTCCGACACAGTCCACCT |
| | GCAGTTCAGCAGCCTGACATCTG |
| | AGGACACTGCCGTCTATTACTGT |
| | GCTAGATTGCACTACTTCGGTAA |
| | TAACTTCTTCTTTGACTACTGGG |
| | GCCAAGGCACCACTCTCACAGTC |
| | TCCTCA (SEQ ID NO: 20) |
| 144L249B | ATGAAATGCAGCGGGGTTATCTT |
| | CTTCCTGATGGCAGTGGTTACAG |
| | GGGTCAACTCAGAGGTTCAGCTG |
| | CAGCAGTCTGGGGCAGAGCTTGT |
| | GAAGCCAGGGGCCTCAGTCAAGT |
| | TGTCCTGCACAGCTTCTGGCTTC |
| | AACATTAAAGACACCTATATATA |
| | CTGGGTGAAGCAGAGGCCTGAAC |
| | AGGGCCTGGAGTGGATTGGAAGG |
| | ATTGATCCTGCGATTGGTACTAC |
| | TAGATATGACCCGAAGTTCCAGG |
| | GCAAGGCCACTCTAACAGCAGAC |
| | ACATCCTCCAACACAGCCTTCCT |
| | GCAGCTCAGCAGCCTGACATCTG |
| | AGGACACTGCCGTCTATTACTGT |
| | GCTAGATATGACTACTCCGGTAG |
| | TAGCTTCTACTTTGACTACTGGG |
| | GCCGAGGCACCACTCTCACAGTC |
| | TCCTCA (SEQ ID NO: 24) |
| 144L124B | ATGAAATGCAGCTGGGTTATCTT |
| | CTTCCTGATGGCAGTGGTTACAG |
| | GGGTCAATTCAGAGGTTCAGCTG |
| | CAGCAGTCTGGGGCAGAGCTTGT |
| | GAAGCCAGGGGCCTCAGTCAAGT |
| | TGTCCTGCACAGCTTCTGGCTTC |
| | AACATTAAAGACACCTATATTTA |
| | CTGGGTGAAGCAGAGGCCTGAAC |
| | AGGGCCTGGAATGGATTGGAAGG |
| | ATTGATCCTGCGAATGGTTATAC |
| | TAGATATGACCCGAAGTTCCAGG |
| | GCAAGGCCACTATGACAGCAGAC |
| | ACATCCTCCAACACAGCCTACCT |
| | GCAGCTCAGCAGCCTGACATCTG |
| | AGGACACTGCCGTCTATTACTGT |
| | GCTAGATATGAATACTACGATAG |
| | TAGCTTCTACTTTGACTACTGGG |
| | GCCAAGGCACCACTCTCACAGTC |
| | TCCTCA (SEQ ID NO: 28) |
| 144L133B | ATGAAGTGCAGCGGGGTTATCTT |
| | CTTCCTGATGGCAGTGGTTACAG |
| | GGGTCAACTCAGAGGTTCAGCTG |
| | CAGCAGTCTGGGGCAGAGCTTGT |
| | GAAGCCAGGGGCCTCAGTCAAGT |
| | TGTCCTGCACAGCTTCTGGCTTC |
| | AACATTAAAGACACCTATATGTA |
| | CTGGGTGAAGCAGAGGCCTGAAC |
| | AGGGCCTGGAGTGGATTGGAAGG |
| | ATTGATCCTGCGATTGGTACTAC |
| | TAGATATGACCCGAAGTTCCAGG |
| | GCAAGGCCACTCTAACAGCAGAC |
| | ACATCCTCCAACACAGCCTTCCT |
| | GCAGCTCAGCAGCCTGACATCTG |
| | AGGACACTGCCGTCTATTACTGT |
| | GCTAGATATGACTACTCCGGTAG |
| | TAGCTTCTACTTTGACTACTGGG |
| | GCCGAGGCACCACTCTCACAGTC |
| | TCCTCA (SEQ ID NO: 32) |
| 144L180A | ATGAAATGCAGCTGGGTTATCTT |
| | CTTCCTGATGGCAGTGGTTACAG |
| | GGGTCAATTCAGAGGTTCAGCTG |
| | CAGCAGTCTGGGGCAGAGCTTGT |
| | GAAGCCAGGGGCCTCAGTCAAGT |
| | TGTCCTGCACAGCTTCTGGCTTC |
| | AACATTAAAGACACCTATATTTA |
| | CTGGGTGAAGCAGAGGCCTGAAC |
| | AGGGCCTGGAATGGATTGGAAGG |
| | ATTGATCCTGCGAATGGTTATAC |
| | TAGATATGACCCGAAGTTCCAGG |
| | GCAAGGCCACTATGACAGCAGAC |
| | ACATCCTCCAACACAGCCTACCT |
| | GCAGCTCAGCAGCCTGACATCTG |
| | AGGACACTGCCGTCTATTACTGT |
| | GCTAGATATGAATACTACGATAG |
| | TAGCTTCTACTTTGACTACTGGG |
| | GCCAAGGCACCACTCTCACAGTC |
| | TCCTCA (SEQ ID NO: 28) |
| 144L472A | ATGAAATGCAGCTGGGTTATCTT |
| | CTTCCTGATGGCAGTGGTTACAG |
| | GGGTCAATTCAGAGGTTCAGCTG |
| | CAGCAGTCTGGGGCAGAGCTTGT |
| | GAAGCCAGGGGCCTCAGTCAAGT |
| | TGTCCTGCACAGTTTCTGGCTTC |
| | AACATTAAAGACACCTATATGTA |
| | CTGGGTGAAGCAGAGGCCTGAAC |
| | AGGGCCTGGAGTGGATTGGAAGG |
| | ATTGATCCTGCGAATGGTTATGC |
| | TAAATATGACCCGAAGTTCCAGG |
| | GCAAGGCCACTTTAACAGCAGAC |
| | ACATCTTCCAATACAGCCTACCT |
| | GCAGCTCAGCAGCCTGACATCTG |
| | AGGACACTGCCGTCTATTTCTGT |
| | GCTAGATTTCATTGGTACGACAG |
| | TGCCTTCTACTTTGACTTCTGGG |
| | GCCAAGGCACCACTCTCACAGTC |
| | TCCTCA (SEQ ID NO: 36) |
| 144D666C | ATGAAATGCAGCTGGGTTATCTT |
| | CTTCCTGATGGCAGTGGTTACAG |
| | GGGTCAATTCAGAGGTTCAGCTG |
| | CAGCAGTCTGGGGCAGAGCTTGT |
| | GAAGCCAGGGGCCTCAGTCAAGT |
| | TGACCTGCACAGCTTCTGGCTTC |
| | AACATTAAAGACACCTATATATA |
| | CTGGGTGAAACAGAGGCCTGCAC |
| | AGGGCCTGGAGTGGATTGGAAGG |
| | ATTGATCCTGCGAATGCTTATAC |
| | TAAATTTGACCCGAAGTTCCAGG |
| | GCAAGGCCACTTTAACAGCAGAC |
| | ACATCCTCCAACACAGCCTACCT |
| | GCAGCTCAGCAGCCTGACATCTG |
| | AGGACACTGCCGTCTATTACTGT |
| | ACTAGATTTCATTGGTACGGTAG |
| | TAGCTTCTTCTTTGACTACTGGG |
| | GCCAAGGCACCACTCTCACAGTC |
| | TCCTCA (SEQ ID NO: 40) |
| 144J171G | ATGAAATGCAGCTGGGTTATCTT |
| | CTTCCTGATGGCAGTGGTTACAG |
| | GGGTCTATTCTGAGGTTCAGCTG |
| | CAGCAGTCTGGGGCAGAGCTTGT |
| | GGAGCCAGGGGCCTCAGTCAAGT |
| | TGTCCTGCACAGCTTCTGGCTTC |
| | AACATTAAAGACACCTACATAAA |
| | CTGGGTGAAGCAGAGGCCTGAAC |
| | AGGGCCTGGAGTGGATTGGAAGG |
| | ATTGATCCTGCGAATGGTTATAC |
| | TAGATATGCCCCGAAGTTCCAGG |
| | GCAAGGCCACTATAACATCAGAC |
| | ACATCCTCCAACACAGCCTACCT |
| | GCAGCTCAGCAGCCTGACATCTG |
| | AGGACGCTGCCGTCTATTCCTGT |
| | TCTACATTAAATTACTACGGTAG |
| | TAGCTTTTTCTTTGACTTCTGGG |
| | GCCAAGGCACCACTCTCACAGTC |
| | TCCTCA (SEQ ID NO: 44) |

TABLE 4
VH nucleic acid sequences, not
including the signal sequences

| | Nucleotide sequences |
|---|---|
| Antibody | (SEQ ID NO:) |
| 144D464A | GAGGTTCAGCTGCAGCAGTCTGGGGCAGA |
| | GCTTGTGAAGCCAGGGGCCTCAGTCAGGT |
| | TGTCCTGCACAGCTTCTGGCTTCAACATT |
| | AAAGACACCTATATACACTGGGTGAAGCA |
| | GAGGCCTGAACAGGGCCTGGAGTGGATTG |
| | GAAGGATTGATCCTGCGATTGGTACTACT |
| | AGATATGACCCGAAGTTCCAGGGCAAGGC |
| | CACTATAACAACAGACACATCCTCCGACA |
| | CAGTCCACCTGCAGTTCAGCAGCCTGACA |
| | TCTGAGGACACTGCCGTCTATTACTGTGC |
| | TAGATTGCACTACTTCGGTAATAACTTCT |
| | TCTTTGACTACTGGGGCCAAGGCACCACT |
| | CTCACAGTCTCCTCA |
| | (SEQ ID NO: 22) |
| 144L249B | GAGGTTCAGCTGCAGCAGTCTGGGGCAGA |
| | GCTTGTGAAGCCAGGGGCCTCAGTCAAGT |
| | TGTCCTGCACAGCTTCTGGCTTCAACATT |
| | AAAGACACCTATATATACTGGGTGAAGCA |
| | GAGGCCTGAACAGGGCCTGGAGTGGATTG |
| | GAAGGATTGATCCTGCGATTGGTACTACT |
| | AGATATGACCCGAAGTTCCAGGGCAAGGC |
| | CACTCTAACAGCAGACACATCCTCCAACA |
| | CAGCCTTCCTGCAGCTCAGCAGCCTGACA |
| | TCTGAGGACACTGCCGTCTATTACTGTGC |
| | TAGATATGACTACTCCGGTAGTAGCTTCT |
| | ACTTTGACTACTGGGGCCGAGGCACCACT |
| | CTCACAGTCTCCTCA |
| | (SEQ ID NO: 26) |
| 144L124B | GAGGTTCAGCTGCAGCAGTCTGGGGCAGA |
| | GCTTGTGAAGCCAGGGGCCTCAGTCAAGT |
| | TGTCCTGCACAGCTTCTGGCTTCAACATT |
| | AAAGACACCTATATTTACTGGGTGAAGCA |
| | GAGGCCTGAACAGGGCCTGGAATGGATTG |
| | GAAGGATTGATCCTGCGAATGGTTATACT |
| | AGATATGACCCGAAGTTCCAGGGCAAGGC |
| | CACTATGACAGCAGACACATCCTCCAACA |
| | CAGCCTACCTGCAGCTCAGCAGCCTGACA |
| | TCTGAGGACACTGCCGTCTATTACTGTGC |
| | TAGATATGAATACTACGATAGTAGCTTCT |
| | ACTTTGACTACTGGGGCCAAGGCACCACT |
| | CTCACAGTCTCCTCA |
| | (SEQ ID NO: 30) |
| 144L133B | GAGGTTCAGCTGCAGCAGTCTGGGGCAGA |
| | GCTTGTGAAGCCAGGGGCCTCAGTCAAGT |
| | TGTCCTGCACAGCTTCTGGCTTCAACATT |
| | AAAGACACCTATATGTACTGGGTGAAGCA |
| | GAGGCCTGAACAGGGCCTGGAGTGGATTG |
| | GAAGGATTGATCCTGCGATTGGTACTACT |
| | AGATATGACCCGAAGTTCCAGGGCAAGGC |
| | CACTCTAACAGCAGACACATCCTCCAACA |
| | CAGCCTTCCTGCAGCTCAGCAGCCTGACA |
| | TCTGAGGACACTGCCGTCTATTACTGTGC |
| | TAGATATGACTACTCCGGTAGTAGCTTCT |
| | ACTTTGACTACTGGGGCCGAGGCACCACT |
| | CTCACAGTCTCCTCA |
| | (SEQ ID NO: 34) |
| 144L180A | GAGGTTCAGCTGCAGCAGTCTGGGGCAGA |
| | GCTTGTGAAGCCAGGGGCCTCAGTCAAGT |
| | TGTCCTGCACAGCTTCTGGCTTCAACATT |
| | AAAGACACCTATATTTACTGGGTGAAGCA |
| | GAGGCCTGAACAGGGCCTGGAATGGATTG |
| | GAAGGATTGATCCTGCGAATGGTTATACT |
| | AGATATGACCCGAAGTTCCAGGGCAAGGC |
| | CACTATGACAGCAGACACATCCTCCAACA |
| | CAGCCTACCTGCAGCTCAGCAGCCTGACA |
| | TCTGAGGACACTGCCGTCTATTACTGTGC |
| | TAGATATGAATACTACGATAGTAGCTTCT |
| | ACTTTGACTACTGGGGCCAAGGCACCACT |
| | CTCACAGTCTCCTCA |
| | (SEQ ID NO: 30) |
| 144L472A | GAGGTTCAGCTGCAGCAGTCTGGGGCAGA |
| | GCTTGTGAAGCCAGGGGCCTCAGTCAAGT |
| | TGTCCTGCACAGTTTCTGGCTTCAACATT |
| | AAAGACACCTATATGTACTGGGTGAAGCA |
| | GAGGCCTGAACAGGGCCTGGAGTGGATTG |
| | GAAGGATTGATCCTGCGAATGGTTATGCT |
| | AAATATGACCCGAAGTTCCAGGGCAAGGC |
| | CACTTTAACAGCAGACACATCTTCCAATA |
| | CAGCCTACCTGCAGCTCAGCAGCCTGACA |
| | TCTGAGGACACTGCCGTCTATTTCTGTGC |
| | TAGATTTCATTGGTACGACAGTGCCTTCT |
| | ACTTTGACTTCTGGGGCCAAGGCACCACT |
| | CTCACAGTCTCCTCA |
| | (SEQ ID NO: 38) |
| 144D666C | GAGGTTCAGCTGCAGCAGTCTGGGGCAGA |
| | GCTTGTGAAGCCAGGGGCCTCAGTCAAGT |
| | TGACCTGCACAGCTTCTGGCTTCAACATT |
| | AAAGACACCTATATATACTGGGTGAAACA |
| | GAGGCCTGCACAGGGCCTGGAGTGGATTG |
| | GAAGGATTGATCCTGCGAATGCTTATACT |
| | AAATTTGACCCGAAGTTCCAGGGCAAGGC |
| | CACTTTAACAGCAGACACATCCTCCAACA |
| | CAGCCTACCTGCAGCTCAGCAGCCTGACA |
| | TCTGAGGACACTGCCGTCTATTACTGTAC |
| | TAGATTTCATTGGTACGGTAGTAGCTTCT |
| | TCTTTGACTACTGGGGCCAAGGCACCACT |
| | CTCACAGTCTCCTCA |
| | (SEQ ID NO: 42) |
| 144J171G | GAGGTTCAGCTGCAGCAGTCTGGGGCAGA |
| | GCTTGTGGAGCCAGGGGCCTCAGTCAAGT |
| | TGTCCTGCACAGCTTCTGGCTTCAACATT |
| | AAAGACACCTACATAAACTGGGTGAAGCA |
| | GAGGCCTGAACAGGGCCTGGAGTGGATTG |
| | GAAGGATTGATCCTGCGAATGGTTATACT |
| | AGATATGCCCCGAAGTTCCAGGGCAAGGC |
| | CACTATAACATCAGACACATCCTCCAACA |
| | CAGCCTACCTGCAGCTCAGCAGCCTGACA |
| | TCTGAGGACGCTGCCGTCTATTCCTGTTC |
| | TACATTAAATTACTACGGTAGTAGCTTTT |
| | TCTTTGACTTCTGGGGCCAAGGCACCACT |
| | CTCACAGTCTCCTCA |
| | (SEQ ID NO: 46) |

TABLE 5
VL nucleic acid sequences,
including the signal sequences

| | Nucleotide sequences |
|---|---|
| Antibody | (SEQ ID NO:) |
| 144D464A | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAGCAGTCACACTCACTTGTCGCTCAAGT |
| | TCTGGGGCTGTTACAACTAGTAACTATGCCAAC |
| | TGGGTCCAAGAAAAACCAGATCATTTATTCGCT |
| | GGTCTAATAGGTGGTACCAACGACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTTCAGCAACCATTGGGTGTTCGGTGGA |
| | GGAACCAAACTGACTGTCCTA |
| | (SEQ ID NO: 48) |
| 144L249B | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTACAGCAACCATTTGGTGTTCGGTGGA |
| | GGAACCAAACTGACTGTCCTA |
| | (SEQ ID NO: 52) |
| 144L124B | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTACAGCAACCATTTGGTGTTCGGTGGA |
| | GGAACCAAACTGACTGTCCTA |
| | (SEQ ID NO: 52) |
| 144L133B | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTACAGCAACCATTTGGTGTTCGGTGGA |
| | GGAACCAAACTGACTGTCCTA |
| | (SEQ ID NO: 52) |
| 144L180A | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTACAGCAACCATTTGGTGTTCGGTGGA |
| | GGAACCAAACTGACTGTCCTA |
| | (SEQ ID NO: 52) |
| 144L472A | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | AGTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGGT |
| | CTATGGTACAGCAACCATTGGGTGTTCGGTGGA |
| | GGAACCAAACTGACTGTCCTA |
| | (SEQ ID NO: 56) |
| 144D666C | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTGTTCACT |
| | GGTCTAATAGGTGGTACCGACAACCGACCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATTTATTTCTGTGCT |
| | CTATGGTACAGCAACCTCTGGGTGTTCGGTGGA |
| | GGAACCAAACTGACTGTCCTA |
| | (SEQ ID NO: 60) |
| 144J171G | ATGGCCTGGATTTCACTTATACTCTCTCTCCTG |
| | GCTCTCAGCTCAGGGGCCATTTCCCAGGCTGTT |
| | GTGACTCAGGAATCTGCACTCACCACATCACCT |
| | GGTGAAACAGTCACACTCACTTGTCGCTCAAGT |
| | ACTGGGGCTGTTACAACTAGTAACTATGCCAAC |
| | TGGGTCCAAGAAAAACCAGATCATTTATTCACT |
| | GGTCTAATAGGTGGTACCAACAACCGAGCTCCA |
| | GGTGTTCCTGCCAGATTCTCAGGCTCCCTGATT |
| | GGAGACAAGGCTGCCCTCACCATCACAGGGGCA |
| | CAGACTGAGGATGAGGCAATATATTTCTGTGCT |
| | CTATGGTACAGCAACCATTGGGTGTTCGGTGGA |
| | GGAACCAAACTGACTGTCCTA |
| | (SEQ ID NO: 64) |

TABLE 6
VL nucleic acid sequences, not
including the signal sequences

| | Nucleotide sequences |
|---|---|
| Antibody | (SEQ ID NO:) |
| 144D464A | CAGGCTGTTGTGACTCAGGAATCTGCACTCACCA |
| | CATCACCTGGTGAAGCAGTCACACTCACTTGTCG |
| | CTCAAGTTCTGGGGCTGTTACAACTAGTAACTAT |
| | GCCAACTGGGTCCAAGAAAAACCAGATCATTTAT |
| | TCGCTGGTCTAATAGGTGGTACCAACGACCGAGC |
| | TCCAGGTGTTCCTGCCAGATTCTCAGGCTCCCTG |
| | ATTGGAGACAAGGCTGCCCTCACCATCACAGGGG |
| | CACAGACTGAGGATGAGGCAATATATTTCTGTGC |
| | TCTATGGTTCAGCAACCATTGGGTGTTCGGTGGA |
| | GGAACCAAACTGACTGTCCTA |
| | (SEQ ID NO: 50) |
| 144L249B | CAGGCTGTTGTGACTCAGGAATCTGCACTCACC |
| | ACATCACCTGGTGAAACAGTCACACTCACTTGT |
| | CGCTCAAGTACTGGGGCTGTTACAACTAGTAAC |
| | TATGCCAACTGGGTCCAAGAAAAACCAGATCAT |
| | TTATTCACTGGTCTAATAGGTGGTACCAACAAC |
| | CGAGCTCCAGGTGTTCCTGCCAGATTCTCAGGC |
| | TCCCTGATTGGAGACAAGGCTGCCCTCACCATC |
| | ACAGGGGCACAGACTGAGGATGAGGCAATATAT |
| | TTCTGTGCTCTATGGTACAGCAACCATTTGGTG |
| | TTCGGTGGAGGAACCAAACTGACTGTCCTA |
| | (SEQ ID NO: 54) |
| 144L124B | CAGGCTGTTGTGACTCAGGAATCTGCACTCAC |
| | CACATCACCTGGTGAAACAGTCACACTCACTT |
| | GTCGCTCAAGTACTGGGGCTGTTACAACTAGT |
| | AACTATGCCAACTGGGTCCAAGAAAAACCAGA |
| | TCATTTATTCACTGGTCTAATAGGTGGTACCA |
| | ACAACCGAGCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATATATTTCTGTGCTCTATGGTACAGCAAC |
| | CATTTGGTGTTCGGTGGAGGAACCAAACTGAC |
| | TGTCCTA |
| | (SEQ ID NO: 54) |
| 144L133B | CAGGCTGTTGTGACTCAGGAATCTGCACTCAC |
| | CACATCACCTGGTGAAACAGTCACACTCACTT |
| | GTCGCTCAAGTACTGGGGCTGTTACAACTAGT |
| | AACTATGCCAACTGGGTCCAAGAAAAACCAGA |
| | TCATTTATTCACTGGTCTAATAGGTGGTACCA |
| | ACAACCGAGCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATATATTTCTGTGCTCTATGGTACAGCAAC |
| | CATTTGGTGTTCGGTGGAGGAACCAAACTGAC |
| | TGTCCTA |
| | (SEQ ID NO: 54) |
| 144L180A | CAGGCTGTTGTGACTCAGGAATCTGCACTCAC |
| | CACATCACCTGGTGAAACAGTCACACTCACTT |
| | GTCGCTCAAGTACTGGGGCTGTTACAACTAGT |
| | AACTATGCCAACTGGGTCCAAGAAAAACCAGA |
| | TCATTTATTCACTGGTCTAATAGGTGGTACCA |
| | ACAACCGAGCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATATATTTCTGTGCTCTATGGTACAGCAAC |
| | CATTTGGTGTTCGGTGGAGGAACCAAACTGAC |
| | TGTCCTA(SEQ ID NO: 54) |
| 144L472A | CAGGCTGTTGTGACTCAGGAATCTGCACTCAC |
| | CACATCACCTGGTGAAACAGTCACACTCACTT |
| | GTCGCTCAAGTAGTGGGGCTGTTACAACTAGT |
| | AACTATGCCAACTGGGTCCAAGAAAAACCAGA |
| | TCATTTATTCACTGGTCTAATAGGTGGTACCA |
| | ACAACCGAGCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATATATTTCTGTGGTCTATGGTACAGCAAC |
| | CATTGGGTGTTCGGTGGAGGAACCAAACTGAC |
| | TGTCCTA |
| | (SEQ ID NO: 58) |
| 144D666C | CAGGCTGTTGTGACTCAGGAATCTGCACTCAC |
| | CACATCACCTGGTGAAACAGTCACACTCACTT |
| | GTCGCTCAAGTACTGGGGCTGTTACAACTAGT |
| | AACTATGCCAACTGGGTCCAAGAAAAACCAGA |
| | TCATTTGTTCACTGGTCTAATAGGTGGTACCG |
| | ACAACCGACCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATTTATTTCTGTGCTCTATGGTACAGCAAC |
| | CTCTGGGTGTTCGGTGGAGGAACCAAACTGAC |
| | TGTCCTA |
| | (SEQ ID NO: 62) |
| 144J171G | CAGGCTGTTGTGACTCAGGAATCTGCACTCAC |
| | CACATCACCTGGTGAAACAGTCACACTCACTT |
| | GTCGCTCAAGTACTGGGGCTGTTACAACTAGT |
| | AACTATGCCAACTGGGTCCAAGAAAAACCAGA |
| | TCATTTATTCACTGGTCTAATAGGTGGTACCA |
| | ACAACCGAGCTCCAGGTGTTCCTGCCAGATTC |
| | TCAGGCTCCCTGATTGGAGACAAGGCTGCCCT |
| | CACCATCACAGGGGCACAGACTGAGGATGAGG |
| | CAATATATTTCTGTGCTCTATGGTACAGCAAC |
| | CATTGGGTGTTCGGTGGAGGAACCAAACTGAC |
| | TGTCCTA |
| | (SEQ ID NO: 66) |

TABLE 7
VH amino acid sequences,
including the signal sequences

| | Amino acid sequences |
|---|---|
| Antibody | (SEQ ID NO:) |
| 144D464A | MKCSWVIFFLMAVVTGVNSEVQLQQS |
| | GAELVKPGASVRLSCTASGFNIKDTY |
| | IHVVVKQRPEQGLEWIGRIDPAIGTT |
| | RYDPKFQGKATITTDTSSDTVHLQFS |
| | SLTSEDTAVYYCARLHYFGNNFFFDY |
| | VVGQGTTLTVSS |
| | (SEQ ID NO: 21) |
| 144L249B | MKCSGVIFFLMAVVTGVNSEVQLQQS |
| | GAELVKPGASVKLSCTASGFNIKDTY |
| | IYVVVKQRPEQGLEWIGRIDPAIGTT |
| | RYDPKFQGKATLTADTSSNTAFLQLS |
| | SLTSEDTAVYYCARYDYSGSSFYFDY |
| | WGRGTTLTVSS |
| | (SEQ ID NO: 25) |
| 144L124B | MKCSWVIFFLMAVVTGVNSEVQLQQS |
| | GAELVKPGASVKLSCTASGFNIKDTY |
| | IYVVVKQRPEQGLEWIGRIDPANGYT |
| | RYDPKFQGKATMTADTSSNTAYLQLS |
| | SLTSEDTAVYYCARYEYYDSSFYFDY |
| | VVGQGTTLTVSS |
| | (SEQ ID NO: 29) |
| 144L133B | MKCSGVIFFLMAVVTGVNSEVQLQQS |
| | GAELVKPGASVKLSCTASGFNIKDTY |
| | MYWVKQRPEQGLEWIGRIDPAIGTTR |
| | YDPKFQGKATLTADTSSNTAFLQLSS |
| | LTSEDTAVYYCARYDYSGSSFYFDYW |
| | GRGTTLTVSS |
| | (SEQ ID NO: 33) |
| 144L180A | MKCSWVIFFLMAVVTGVNSEVQLQQS |
| | GAELVKPGASVKLSCTASGFNIKDTY |
| | IYVVVKQRPEQGLEWIGRIDPANGYT |
| | RYDPKFQGKATMTADTSSNTAYLQLS |
| | SLTSEDTAVYYCARYEYYDSSFYFDY |
| | VVGQGTTLTVSS |
| | (SEQ ID NO: 29) |
| 144L472A | MKCSWVIFFLMAVVTGVNSEVQLQQS |
| | GAELVKPGASVKLSCTVSGFNIKDTY |
| | MYWVKQRPEQGLEWTGRIDPANGYAK |
| | YDPKFQGKATLTADTSSNTAYLQLSS |
| | LTSEDTAVYFCARFHVVYDSAFYFDF |
| | WGQGTTLTVSS |
| | (SEQ ID NO: 37) |
| 144D666C | MKCSWVIFFLMAVVTGVNSEVQLQQS |
| | GAELVKPGASVKLTCTASGFNIKDTY |
| | IYVVVKQRPAQGLEWIGRIDPANAYT |
| | KFDPKFQGKATLTADTSSNTAYLQLS |
| | SLTSEDTAVYYCTRFHWYGSSFFFDY |
| | VVGQGTTLTVSS |
| | (SEQ ID NO: 41) |
| 144J171G | MKCSWVIFFLMAVVTGVYSEVQLQQS |
| | GAELVEPGASVKLSCTASGFNIKDTY |
| | INVVVKQRPEQGLEWIGRIDPANGYT |
| | RYAPKFQGKATITSDTSSNTAYLQLS |
| | SLTSEDAAVYSCSTLNYYGSSFFFDF |
| | WGQGTTLTVSS |
| | (SEQ ID NO: 45) |

TABLE 8
VH amino acid sequences, not
including the signal sequences

| | Amino acid sequences |
|---|---|
| Antibody | (SEQ ID NO:) |
| 144D464A | EVQLQQSGAELVKPGASVRLSCTASG |
| | FNIKDTYIHVVVKQRPEQGLEWIGRI |
| | DPAIGTTRYDPKFQGKATITTDTSSD |
| | TVEILQFSSLTSEDTAVYYCARLHYF |
| | GNNFFFDYVVGQGTTLTVSS |
| | (SEQ ID NO: 23) |
| 144L249B | EVQLQQSGAELVKPGASVKLSCTASG |
| | FNIKDTYIYVVVKQRPEQGLEWIGRI |
| | DPAIGTTRYDPKFQGKATLTADTSSN |
| | TAFLQLSSLTSEDTAVYYCARYDYSG |
| | SSFYFDYVVGRGTTLTVSS |
| | (SEQ ID NO: 27) |
| 144L124B | EVQLQQSGAELVKPGASVKLSCTASG |
| | FNIKDTYIYVVVKQRPEQGLEWIGRI |
| | DPANGYTRYDPKFQGKATMTADTSSN |
| | TAYLQLSSLTSEDTAVYYCARYEYYD |
| | SSFYFDYVVGQGTTLTVSS |
| | (SEQ ID NO: 31) |
| 144L133B | EVQLQQSGAELVKPGASVKLSCTASG |
| | FNIKDTYMYVVVKQRPEQGLEW1GRI |
| | DPAIGTTRYDPKFQGKATLTADTSSN |
| | TAFLQLSSLTSEDTAVYYCARYDYSG |
| | SSFYFDYVVGRGTTLTVSS |
| | (SEQ ID NO: 35) |
| 144L180A | EVQLQQSGAELVKPGASVKLSCTASG |
| | FNIKDTYIYVVVKQRPEQGLEWIGRI |
| | DPANGYTRYDPKFQGKATMTADTSSN |
| | TAYLQLSSLTSEDTAVYYCARYEYYD |
| | SSFYFDYVVGQGTTLTVSS |
| | (SEQ ID NO: 31) |
| 144L472A | EVQLQQSGAELVKPGASVKLSCTVSG |
| | FNIKDTYMYVVVKQRPEQGLEWIGRI |
| | DPANGYAKYDPKFQGKATLTADTSSN |
| | TAYLQLSSLTSEDTAVYFCARFHVVY |
| | DSAFYFDFWGQGTTLTVSS |
| | (SEQ ID NO: 39) |
| 144D666C | EVQLQQSGAELVKPGASVKLTCTASG |
| | FNIKDTYIYVVVKQRPAQGLEWIGRI |
| | DPANAYTKFDPKFQGKATLTADTSSN |
| | TAYLQLSSLTSEDTAVYYCTRFHVVY |
| | GSSFFFDYVVGQGTTLTVSS |
| | (SEQ ID NO: 43) |
| 144J171G | EVQLQQSGAELVEPGASVKLSCTASG |
| | FNIKDTYINVVVKQRPEQGLEWIGRI |
| | DPANGYTRYAPKFQGKATITSDTSSN |
| | TAYLQLSSLTSEDAAVYSCSTLNYYG |
| | SSFFFDFWGQGTTLTVSS |
| | (SEQ ID NO: 47) |

TABLE 9
VL amino acid sequences,
including the signal sequences

| | Amino acid sequences |
|---|---|
| Antibody | (SEQ ID NO:) |
| 144D464A | MAWISLILSLLALSSGAISQAVVTQES |
| | ALTTSPGEAVTLTCRSSSGAVTTSNYA |
| | NVVVQEKPDHLFAGLIGGTNDRAPGVP |
| | ARFSGSLIGDKAALTITGAQTEDEAIY |
| | FCALWFSNHVVVFGGGTKLTVL |
| | (SEQ ID NO: 49) |
| 144L249B | MAWISLILSLLALSSGAISQAVVTQES |
| | ALTTSPGETVTLTCRSSTGAVTTSNYA |
| | NVVVQEKPDHLFTGLIGGTNNRAPGVP |
| | ARFSGSLIGDKAALTITGAQTEDEAIY |
| | FCALWYSNEILVFGGGTKLTVL |
| | (SEQ ID NO: 53) |
| 144L124B | MAWISLILSLLALSSGAISQAVVTQES |
| | ALTTSPGETVTLTCRSSTGAVTTSNYA |
| | NVVVQEKPDHLFTGLIGGTNNRAPGVP |
| | ARFSGSLIGDKAALTITGAQTEDEAIY |
| | FCALWYSNEILVFGGGTKLTVL |
| | (SEQ ID NO: 53) |
| 144L133B | MAWISLILSLLALSSGAISQAVVTQES |
| | ALTTSPGETVTLTCRSSTGAVTTSNYA |
| | NVVVQEKPDHLFTGLIGGTNNRAPGVP |
| | ARFSGSLIGDKAALTITGAQTEDEAIY |
| | FCALWYSNEILVFGGGTKLTVL |
| | (SEQ ID NO: 53) |
| 144L180A | MAWISLILSLLALSSGAISQAVVTQES |
| | ALTTSPGETVTLTCRSSTGAVTTSNYA |
| | NVVVQEKPDHLFTGLIGGTNNRAPGVP |
| | ARFSGSLIGDKAALTITGAQTEDEAIY |
| | FCALWYSNEILVFGGGTKLTVL |
| | (SEQ ID NO: 53) |
| 144L472A | MAWISLILSLLALSSGAISQAVVTQES |
| | ALTTSPGETVTLTCRSSSGAVTTSNYA |
| | NVVVQEKPDHLFTGLIGGTNNRAPGVP |
| | ARFSGSLIGDKAALTITGAQTEDEAIY |
| | FCGLWYSNHVVVFGGGTKLTVL |
| | (SEQ ID NO: 57) |
| 144D666C | MAWISLILSLLALSSGAISQAVVTQES |
| | ALTTSPGETVTLTCRSSTGAVTTSNYA |
| | NVVVQEKPDHLFTGLIGGTDNRPPGVP |
| | ARFSGSLIGDKAALTITGAQTEDEAIY |
| | FCALWYSNLWVFGGGTKLTVL |
| | (SEQ ID NO: 61) |
| 144J171G | MAWISLILSLLALSSGAISQAVVTQES |
| | ALTTSPGETVTLTCRSSTGAVTTSNYA |
| | NVVVQEKPDHLFTGLIGGTNNRAPGVP |
| | ARFSGSLIGDKAALTITGAQTEDEAIY |
| | FCALWYSNHVVVFGGGTKLTVL |
| | (SEQ ID NO: 65) |

TABLE 10
VL amino acid sequences, not
including the signal sequences

| | Amino acid sequences |
|---|---|
| Antibody | (SEQ ID NO:) |
| 144D464A | QAVVTQESALTTSPGEAVTLTCRSSS |
| | GAVTTSNYANVVVQEKPDHLFAGLIG |
| | GTNDRAPGVPARFSGSLIGDKAALTI |
| | TGAQIEDEAIYFCALWFSNHVVVFGG |
| | GTKLTVL (SEQ ID NO: 51) |
| 144L249B | QAVVTQESALTTSPGETVTLTCRSST |
| | GAVTTSNYANVVVQEKPDHLFTGLIG |
| | GTNNRAPGVPARFSGSLIGDKAALTI |
| | TGAQIEDEAIYFCALWYSNEILVFGG |
| | GTKLTVL(SEQ ID NO: 55) |
| 144L124B | QAVVTQESALTTSPGETVTLTCRSST |
| | GAVTTSNYANVVVQEKPDHLFTGLIG |
| | GTNNRAPGVPARFSGSLIGDKAALTI |
| | TGAQIEDEAIYFCALWYSNEILVFGG |
| | GTKLTVL(SEQ ID NO: 55) |
| 144L133B | QAVVTQESALTTSPGETVTLTCRSST |
| | GAVTTSNYANVVVQEKPDHLFTGLIG |
| | GTNNRAPGVPARFSGSLIGDKAALTI |
| | TGAQIEDEAIYFCALWYSNEILVFGG |
| | GTKLTVL(SEQ ID NO: 55) |
| 144L180A | QAVVTQESALTTSPGETVTLTCRSST |
| | GAVTTSNYANVVVQEKPDHLFTGLIG |
| | GTNNRAPGVPARFSGSLIGDKAALTI |
| | TGAQIEDEAIYFCALWYSNEILVFGG |
| | GTKLTVL(SEQ ID NO: 55) |
| 144L472A | QAVVTQESALTTSPGETVTLTCRSSS |
| | GAVTTSNYANVVVQEKPDHLFTGLIG |
| | GTNNRAPGVPARFSGSLIGDKAALTI |
| | TGAQTEDEAIYFCGLWYSNHVVVFGG |
| | GTKLTVL(SEQ ID NO: 59) |
| 144D666C | QAVVTQESALTTSPGETVTLTCRSST |
| | GAVTTSNYANVVVQEKPDHLFTGLIG |
| | GTDNRPPGVPARFSGSLIGDKAALTI |
| | TGAQTEDEAIYFCALWYSNLWVFGGG |
| | TKLTVL (SEQ ID NO: 63) |
| 144J171G | QAVVTQESALTTSPGETVTLTCRSST |
| | GAVTTSNYANVVVQEKPDHLFTGLIG |
| | GTNNRAPGVPARFSGSLIGDKAALTI |
| | TGAQTEDEAIYFCALWYSNHVVVFGG |
| | GTKLTVL(SEQ ID NO: 67) |

The amino acid sequences of the CDR regions of the identified eight antibodies according to Kabat numbering are listed in Table 11 and Table 12 below.

TABLE 11
VH CDR amino acid sequences

| | VH CDR1 | VH CDR2 | VH CDR3 |
|---|---|---|---|
| | (SEQ ID | (SEQ ID | (SEQ ID |
| Antibody | NO:) | NO:) | NO:) |
| 144D464A | DTYIH | RIDPAIG | LHYFGN |
| | (SEQ ID | TTRYDPK | NFFFDY |
| | NO: 68) | FQG | (SEQ ID |
| | | (SEQ ID | NO: 70) |
| | | NO: 69) | |
| 144L249B | DTYIY | RIDPAIG | YDYSGS |
| | (SEQ ID | TTRYDPK | SFYFDY |
| | NO: 71) | FQG | (SEQ ID |
| | | (SEQ ID | NO: 72) |
| | | NO: 69) | |
| 144L124B | DTYIY | RIDPANG | YEYYDS |
| | (SEQ ID | YTRYDPK | SFYFDY |
| | NO: 71) | FQG | (SEQ ID |
| | | (SEQ ID | NO: 74) |
| | | NO: 73) | |
| 144L133B | DTYMY | RIDPAIG | YDYSGS |
| | (SEQ ID | TTRYDPK | SFYFDY |
| | NO: 75) | FQG | (SEQ ID |
| | | (SEQ ID | NO: 72) |
| | | NO: 69) | |
| 144L180A | DTYIY | RIDPANG | YEYYDS |
| | (SEQ ID | YTRYDPK | SFYFDY |
| | NO: 71) | FQG | (SEQ ID |
| | | (SEQ ID | NO: 74) |
| | | NO: 73) | |
| 144L472A | DTYMY | RIDPANG | FHVVYDS |
| | (SEQ ID | YAKYDPK | AFYFDF |
| | NO: 75) | FQG | (SEQ ID |
| | | (SEQ ID | NO: 77) |
| | | NO: 76) | |
| 144D666C | DTYIY | RIDPANA | FHWYGSS |
| | (SEQ ID | YTKFDPK | FFFDY |
| | NO: 71) | FQG | (SEQ ID |
| | | (SEQ ID | NO: 79) |
| | | NO: 78) | |
| 144J171G | DTYIN | RIDPANG | LNYYGSS |
| | (SEQ ID | YTRYAPK | FFFDF |
| | NO: 80) | FQG | (SEQ ID |
| | | (SEQ ID | NO: 82) |
| | | NO: 81) | |

TABLE 12
VL CDR amino acid sequences

| | VL CDR1 | VL CDR2 | VL CDR3 |
|---|---|---|---|
| | (SEQ ID | (SEQ ID | (SEQ ID |
| Antibody | NO:) | NO:) | NO:) |
| 144D464A | RSSSGAV | GTNDRAP | ALWFS |
| | TTSNYAN | (SEQ ID | NHVVV |
| | (SEQ ID | NO: 84) | (SEQ ID |
| | NO: 83) | | NO: 85) |
| 144L249B | RSSTGAV | GTNNRAP | ALWYS |
| | TTSNYAN | (SEQ ID | NEILV |
| | (SEQ ID | NO: 87) | (SEQ ID |
| | NO: 86) | | NO: 88) |
| 144L124B | RSSTGAV | GTNNRAP | ALWYS |
| | TTSNYAN | (SEQ ID | NEILV |
| | (SEQ ID | NO: 87) | (SEQ ID |
| | NO: 86) | | NO: 88) |
| 144L133B | RSSTGAV | GTNNRAP | ALWYS |
| | TTSNYAN | (SEQ ID | NEILV |
| | (SEQ ID | NO: 87) | (SEQ ID |
| | NO: 86) | | NO: 88) |
| 144L180A | RSSTGAV | GTNNRAP | ALWYS |
| | TTSNYAN | (SEQ ID | NEILV |
| | (SEQ ID | NO: 87) | (SEQ ID |
| | NO: 86) | | NO: 88) |
| 144L472A | RSSSGAV | GTNNRAP | GLWYS |
| | TTSNYAN | (SEQ ID | NHVVV |
| | (SEQ ID | NO: 87) | (SEQ ID |
| | NO: 83) | | NO: 89) |
| 144D666C | RSSTGAV | GTDNRPP | ALWYS |
| | TTSNYAN | (SEQ ID | NLWV |
| | (SEQ ID | NO: 90) | (SEQ ID |
| | NO: 86) | | NO: 91) |
| 144J171G | RSSTGAV | GTNNRAP | ALWYS |
| | TTSNYAN | (SEQ ID | NHVVV |
| | (SEQ ID | NO: 87) | (SEQ ID |
| | NO: 86) | | NO: 92) |

6.2.2 Construction of Expression Vectors for Production of Recombinant Antibodies

Vectors for mammalian expression of chimeric mouse/human antibodies were generated by ligating PCR-amplified DNA coding for the VH or VL of a specific antibody clone into separate, linearized eukaryotic expression vectors, between 5′ cDNA coding for a mouse IgG signal peptide and 3′ cDNA coding for either human IgG1 or human Ig Lambda constant domains, respectively. Ligation was performed using a GeneArt Seamless Cloning and Assembly Kit (cat #A13288, Invitrogen, Carlsbad, Calif.), as per the product manual. DNA plasmids were amplified as before, and the entire heavy or light chain cDNA sequence was validated by Sanger sequencing. Chimeric mouse/human IgG1 heavy chain sequences are represented by joining a clonal VH nucleotide or amino acid sequence to a human IgG1 constant nucleotide sequence (SEQ ID NO: 93), or amino acid sequence (SEQ ID NO: 94), respectively, and complete chimeric mouse/human Lambda light chain sequences are represented by joining a clonal VL nucleotide or amino acid sequence to human IgLambda (IGLC2) constant nucleotide sequence (SEQ ID NO: 95), or amino acid sequence (SEQ ID NO: 96), respectively.

The sequences of SEQ ID NO: 93, SEQ ID NO: 94, SEQ ID NO: 95, and SEQ ID NO: 96 are listed below:

Human IgG1 constant nucleotide
sequence (SEQ ID NO: 93)
(SEQ ID NO: 93)
GCTAGCACCAAGGGCCCATCGGTCTTCCCCC
TGGCACCCTCCTCCAAGAGCACCTCTGGGGG
CACAGCGGCCCTGGGCTGCCTGGTCAAGGAC
TACTTCCCCGAACCGGTGACGGTGTCGTGGA
ACTCAGGCGCCCTGACCAGCGGCGTGCACAC
CTTCCCGGCTGTCCTACAGTCCTCAGGACTC
TACTCCCTCAGCAGCGTGGTGACCGTGCCCT
CCAGCAGCTTGGGCACCCAGACCTACATCTG
CAACGTGAATCACAAGCCCAGCAACACCAAG
GTGGACAAGAAAGTTGAGCCCAAATCTTGTG
ACAAAACTCACACATGCCCACCGTGCCCAGC
ACCTGAACTCCTGGGGGGACCGTCAGTCTTC
CTCTTCCCCCCAAAACCCAAGGACACCCTCA
TGATCTCCCGGACCCCTGAGGTCACATGCGT
GGTGGTGGACGTGAGCCACGAAGACCCTGAG
GTCAAGTTCAACTGGTACGTGGACGGCGTGG
AGGTGCATAATGCCAAGACAAAGCCGCGGGA
GGAGCAGTACAACAGCACGTACCGTGTGGTC
AGCGTCCTCACCGTCCTGCACCAGGACTGGC
TGAATGGCAAGGAGTACAAGTGCAAGGTCTC
CAACAAAGCCCTCCCAGCCCCCATCGAGAAA
ACCATCTCCAAAGCCAAAGGGCAGCCCCGAG
AACCACAGGTGTACACCCTGCCCCCATCCCG
GGATGAGCTGACCAAGAACCAGGTCAGCCTG
ACCTGCCTGGTCAAAGGCTTCTATCCCAGCG
ACATCGCCGTGGAGTGGGAGAGCAATGGGCA
GCCGGAGAACAACTACAAGACCACGCCTCCC
GTGCTGGACTCCGACGGCTCCTTCTTCCTCT
ACAGCAAGCTCACCGTGGACAAGAGCAGGTG
GCAGCAGGGGAACGTCTTCTCATGCTCCGTG
ATGCATGAGGCTCTGCACAACCACTACACGC
AGAAGAGCCTCTCCCTGTCTCCGGGTAAATG
A.
Human IgG1 constant amino acid
sequence (SEQ ID NO: 94)
(SEQ ID NO: 94)
ASTKGPSVFPLAPSSKSTSGGTAALGCLVKD
YFPEPVTVSWNSGALTSGVHTFPAVLQSSGL
YSLSSVVTVPSSSLGTQTYICNVNHKPSNTK
VDKKVEPKSCDKTHTCPPCPAPELLGGPSVF
LFPPKPKDTLMISRTPEVTCVVVDVSHEDPE
VKFNVVYVDGVEVHNAKTKPREEQYNSTYRV
VSVLTVLHQDWLNGKEYKCKVSNKALPAPIE
KTISKAKGQPREPQVYTLPPSRDELTKNQVS
LTCLVKGFYPSDIAVEWESNGQPENNYKTTP
PVLDSDGSFFLYSKLTVDKSRWQQGNVFSCS
VMHEALHNHYTQKSLSLSPGK
Human IgLambda (IGLC2) constant
nucleotide sequence (SEQ ID NO: 95)
(SEQ ID NO: 95)
GGTCAGCCCAAGGCTGCCCCCTCGGTCACTC
TGTTCCCGCCCTCCTCTGAGGAGCTTCAAGC
CAACAAGGCCACACTGGTGTGTCTCATAAGT
GACTTCTACCCGGGAGCCGTGACAGTGGCCT
GGAAGGCAGATAGCAGCCCCGTCAAGGCGGG
AGTGGAGACCACCACACCCTCCAAACAAAGC
AACAACAAGTACGCGGCCAGCAGCTACCTGA
GCCTGACGCCTGAGCAGTGGAAGTCCCACAG
AAGCTACAGCTGCCAGGTCACGCATGAAGGG
AGCACCGTGGAGAAGACAGTGGCCCCTACAG
AATGTTCATGA
Human IgLambda (IGLC2) constant
amino acid sequence (SEQ ID NO: 96)
(SEQ ID NO: 96)
GQPKAAPSVTLFPPSSEELQANKATLVCLIS
DFYPGAVTVAWKADSSPVKAGVETTTPSKQS
NNKYAASSYLSLTPEQWKSHRSYSCQVTHEG
STVEKTVAPTECS.

### 6.2.3 Antibody Production

Production of hybridoma derived antibodies was performed by expansion of the antibody-producing hybridoma lines to 50 ml-200 ml cultures in media supplemented with ultra-low Ig fetal bovine serum (cat# 16250078, Life Technologies, Carlsbad, Calif.).

Production of recombinant antibodies from mammalian cells was performed by transient transfection of Expi293F cells (cat #A14528, Life Technologies, Carlsbad, Calif.) with antibody expression vectors using the Expi293 Expression System (cat #A14524, Life Technologies, Carlsbad, Calif.). Briefly, separate mammalian expression vectors for antibody heavy chain and antibody light chain were combined in a 1:1 or 1:3 ratio, heavy chain to light chain, and transfected using the methods described in the manufacturer's manual. Five to seven days after transfection, culture media was collected and clarified by centrifugation followed by 0.22 micron filtration (Stericup® Filter Units, cat # SCGPUO5RE, EMD Millipore, Temecula, Calif.).

### 6.2.4 Purification of Antibodies

Hybridoma-derived or recombinant monoclonal antibodies were purified from culture media using recombinant MabS elect SuRe Protein A affinity resin (cat# 28408253, GE Healthcare Life Sciences, Pittsburgh, Pa.). The conditioned medium was filtered with a 0.22 μm vacuum filter unit (Millipore, Bedford, Mass.) and loaded onto a HiTrap MabSelect SuRe column (cat# 28408253, GE Healthcare Life Sciences, Pittsburgh, Pa.) of appropriate capacity to match the amount of antibody in the medium. The column was washed thoroughly with 6 column volumes of PBS, the antibody was eluted with 0.1 M Gly-HCl, 0.15 M NaCl pH 3.7 (10 CV) followed by 0.1 M Gly-HCl, 0.15 M NaCl pH 2.5 (6 CV), and neutralized with 1 M Tris-HCl, pH 8.0. The fractions were analyzed by SDS-PAGE and the positive fractions were pooled and dialyzed against PBS pH 7.4 (cat# P3813, Sigma Aldrich, St. Louis, Mo.). Following dialysis, antibody samples were concentrated with a centrifugal filter concentrator (Vivaspin, 30,000 MWCO, Cat#VS2022, Sartorius, Goettingen, Germany). Finally, the antibody was filter sterilized using syringe filters with 0.22 μm pore diameter and the antibody concentration was determined by the Lowry method. Pyrogen content was determined using FDA-licensed Endosafe-PTS Limulus Amebocyte Lysate (LAL) assay (Charles River Laboratories, San Diego, Calif.). The limits of detection of this assay are 1-0.01 EU/mL of endotoxin. If the test was negative, the samples were considered endotoxin free.

### 6.2.5 Fab Generation

Fab fragments of IL36α and IL36γ dual antagonist antibody 144D464A were enzymatically prepared using Fab Preparation Kit (cat# 44985, Thermo Fisher, Waltham, Mass.) according to the manufacturer instructions. After papain digestion, the sample was loaded onto a HiTrap MabSelect SuRe column (cat# 28408253, GE Healthcare Life Sciences, Pittsburgh, Pa.) to remove undigested antibody and Fc fragments. The flow-through fractions containing Fab fragments were analyzed by SDS-PAGE and the positive fractions were pooled and dialyzed against PBS pH 7.4 (cat# P3813, Sigma Aldrich, St. Louis, Mo.). Following dialysis, protein sample was concentrated with a centrifugal filter concentrator (Vivaspin 3,000 MWCO, cat# VS2091, Sartorius, Goettingen, Germany). The Fab fragment was further purified by size exclusion chromatography, to remove any contaminants or degradation products. The fractions were analyzed by SDS-PAGE and the positive fractions were pooled and dialyzed against PBS pH 7.4 (cat# P3813, Sigma Aldrich, St. Louis, Mo.).

### 6.3 Example 3—Analysis of Potency in Primary Cell Assays

The monoclonal antibodies identified as IL-36α and IL-36γ dual-antagonists were evaluated for functional potency in primary human keratinocyte assays. Human neonatal keratinocytes (Cat#102-05n, Cell Applications, San Diego, Calif.) were cultured in keratinocyte growth media (Cat#141-500, Cell Applications, San Diego, Calif.). For evaluation of the IC₅₀ potency of the IL-36 monoclonal antibodies in antagonizing human IL-36α, IL-36β, and IL-36γ, purified monoclonal antibodies were diluted into HaCaT culture media to a 600 nM concentration followed by a 2-fold dilution series. 10 μL of diluted antibodies were transferred to the assay plate for a final assay concentration starting at 200 nM, followed by a 2-fold dilution series. IL-36Ra was utilized as a positive control for IL-36 receptor antagonism and was handled in a similar fashion as the IL-36 monoclonal antibodies, though the final assay concentration often started at 1000 nM, followed by a 2-fold dilution series. For a given experiment, assay culture supernatants were diluted by either 2-fold or 5-fold for measurement of secreted IL-8 (Cat# 88-8086-88, Life Technologies, Carlsbad, Calif.). O.D. 450 values from the IL-8 ELISA were graphed and IC₅₀ values were calculated using GraphPad PRISM™ software.

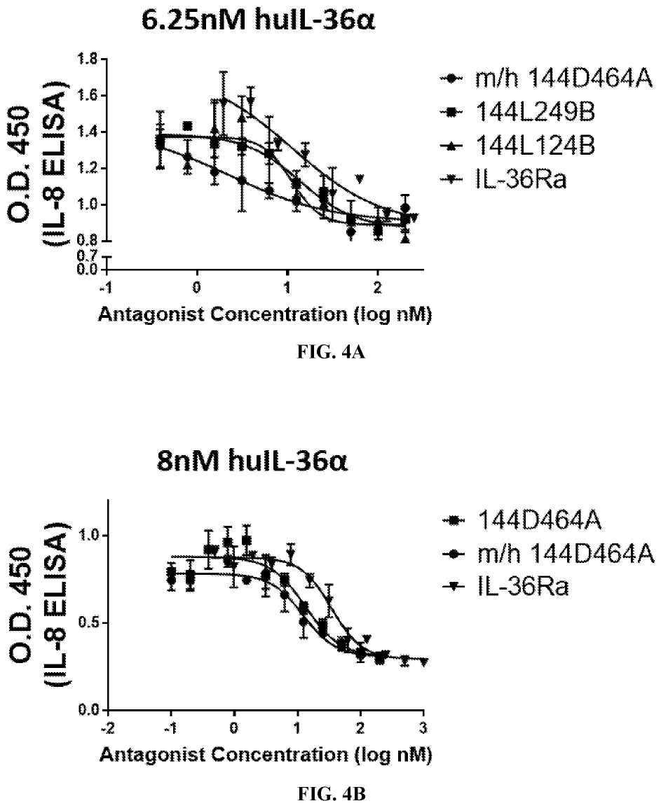

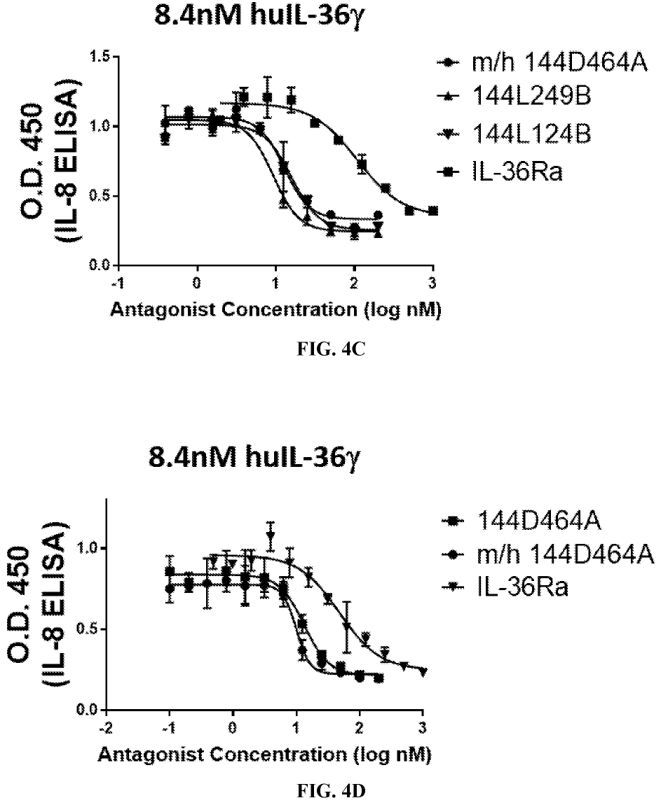

In primary human keratinocyte assays, the antibodies tested here were shown to antagonize both IL-36α and IL-36γ and the potency of this activity was frequently greater than that observed for IL-36Ra (see FIG. 4A to 4D, Table 13).

TABLE 13
IC₅₀ values of antibodies and IL-36Ra antagonizing human IL-36α
and IL-36γ in primary human keratinocyte functional assays

| | FIG. 4A | FIG. 4B | FIG. 4C | FIG. 4D |
|---|---|---|---|---|
| | 6.25 nM | 8 nM | 8.4 nM | 8.4 nM |
| IC₅₀ | human | human | human | human |
| (nM) | IL-36α | IL-36α | IL-36γ | IL-36γ |
| 144D464A | n.t | 13.45 | n.t | 13.31 |
| m/h | 2.317 | 12.29 | 12.84 | 10.02 |
| 144D464A | | | | |
| 144L249B | 13.78 | n.t | 9.275 | n.t |
| 144L124B | 11.09 | n.t | 15.82 | n.t |
| IL-36Ra | 11.24 | 34.59 | 108.6 | 47.93 |

Note:
n.t. means not tested

IL-36 antagonist monoclonal antibodies were also evaluated for functional potency in primary human monocytes assays. Human peripheral blood mononuclear cells were isolated from fresh whole blood from healthy human donors by conventional Ficoll gradient (GE Healthcare, Cat# 17144003, Marlborough, Mass.). Monocytes were enriched with an EasySep Monocyte Enrichment Kit using negative selection (Cat# 19059, STEMCELL Technologies, Tukwila, Wash.) and resuspended at a concentration of 2.5×10⁶ cells/ml in monocyte culture media [RPMI (Cat#21870-016, Life Technologies, Carlsbad, Calif.), 5% FCS (Cat# SH30071.03, Hyclone, Logan, Utah), 1 mM Sodium Pyruvate (Cat# 13-115E, Lonza, Walkersville, Md.), 100 U/ml Penicillin and 100 μg/ml Streptomycin (Cat# P0781, Sigma, St. Louis, Mo.), 1×Glutamax (Cat# 35050-061, Life Technologies, Carlsbad, Calif.), and 1×non-essential amino acids (Cat# 13-114E, Lonza, Walkersville, Md.)]. 10 μL of IL-36α or IL-36γ cytokines diluted in monocyte culture media were added to 384-well assay plates. Final assay concentrations of IL-36 cytokines ranged from 6.25 nM to 50 nM. Purified monoclonal antibodies were diluted into monocyte culture media to a 600 nM concentration followed by a 2-fold dilution series. 10 μL of diluted antibodies were transferred to the assay plate for a final assay concentration starting at 200 nM, followed by a 2-fold dilution series. IL-36Ra was utilized as a positive control for IL-36 receptor antagonism and was handled in a similar fashion as the IL-36 monoclonal antibodies, though the final assay concentration often started at 1000 nM, followed by a 2-fold dilution series. 10 μL of the monocyte cell suspension was added to the plates for a final cell number of 25,000 cells/well. Assay plates were incubated at 5% CO₂, 37° C. for 20 hours. For a given experiment, assay culture supernatants were diluted either 2-fold, 2.5-fold, or 5-fold for measurement of secreted IL-8 (Cat# 88-8086-88, Life Technologies, Carlsbad, Calif.). O.D. 450 values from the IL-8 ELISA were graphed and IC₅₀ values were calculated using GraphPad PRISM™ software.

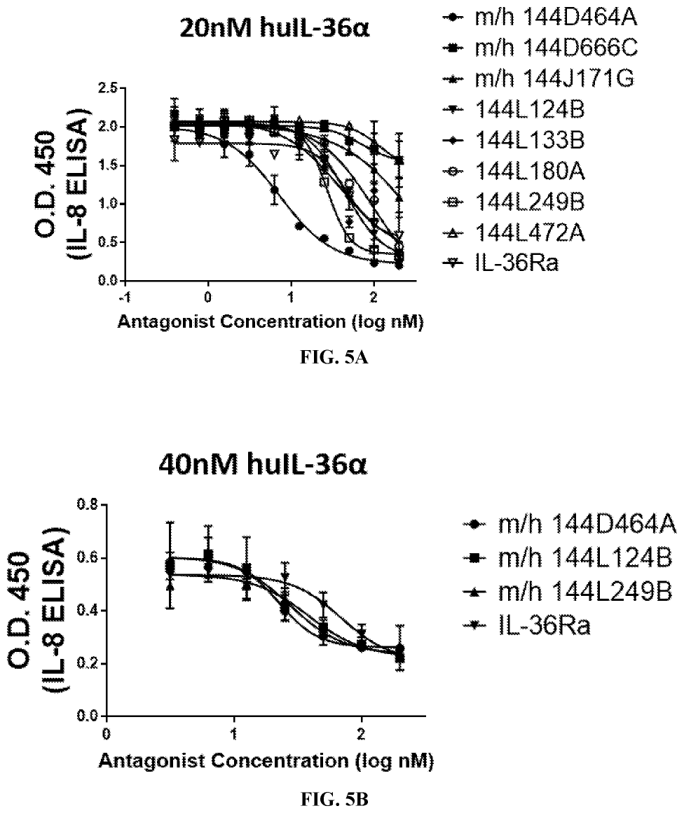

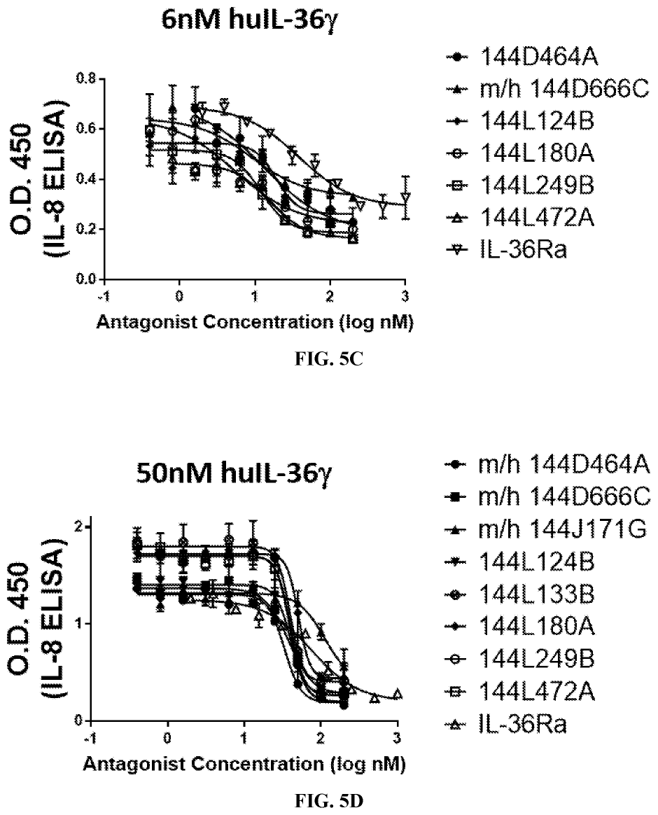

The tested antibodies antagonized both IL-36α and IL-36γ and the potency of this activity was frequently greater than that observed for IL-36Ra (see FIG. 5A to 5D, Table 14).

TABLE 14
IC₅₀ values of antibodies and IL-36Ra antagonizing human IL-36α
and IL-36γ in primary human monocyte functional assays

| | FIG. 5A | FIG. 5B | FIG. 5C | FIG. 5D |
|---|---|---|---|---|
| | 20 nM | 40 nM | 6 nM | 50 nM |
| IC₅₀ | human | human | human | human |
| (nM) | IL-36α | IL-36α | IL-36γ | IL-36γ |
| 144D464A | n.t | n.t | 13.21 | n.t |
| m/h | 7.154 | 22.44 | n.t | 33.34 |
| 144D464A | | | | |
| m/h | weak | n.t | 9.019 | 38.11 |
| 144D666C | | | | |
| 144J171G | weak | n.t | n.t | 113.3 |
| 144L124B | 44.21 | n.t | 19.14 | 41.25 |
| m/h | n.t | 27.46 | n.t | n.t |
| 144L124B | | | | |
| 144L133B | 39.46 | n.t | n.t | 39.6 |
| 144L180A | 96.88 | n.t | 5.486 | 50.89 |
| 144L249B | 27.06 | n.t | 11.96 | 38.69 |
| m/h | n.t | 38.32 | n.t | n.t |
| 144L249B | | | | |
| 144L472A | Weak | n.t | 15.74 | 40.02 |
| IL-36Ra | 46.54 | 69.13 | 37.95 | 76.31 |

Note:
n.t. means not tested.

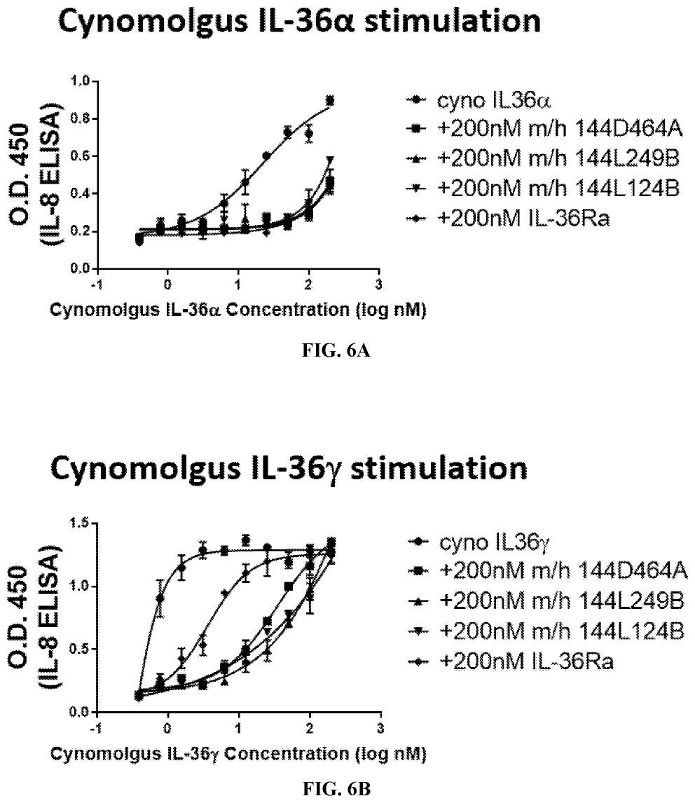

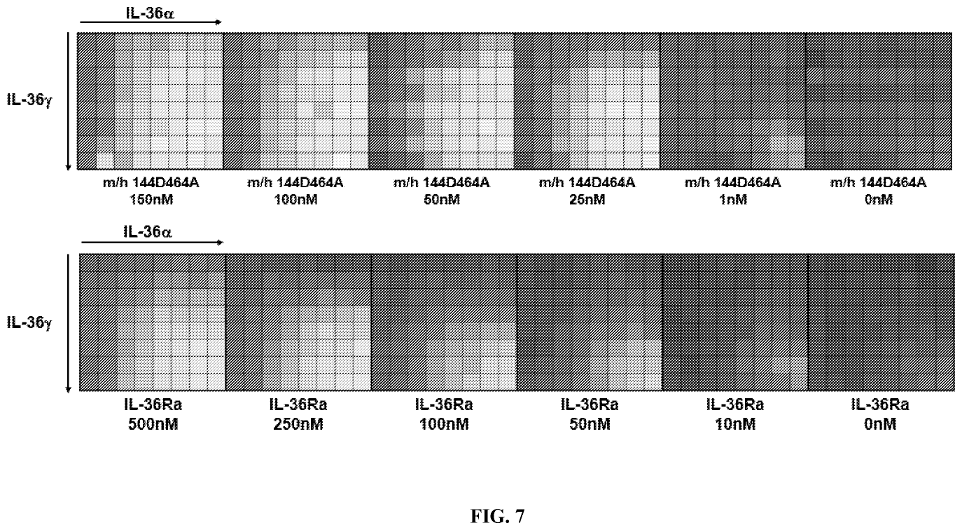

A primary cynomolgus keratinocyte assay was utilized to evaluate the antagonist activity of selected antibodies. Primary cynomolgus keratinocytes (Cat# CM-C-KRT, Zen-Bio, Research Triangle Park, N.C.) were cultured in CellnTec Medium (Cat#CnT-07, CellnTec, Bern, Switzerland). Cells were stimulated with a titration of cynomolgus IL-36α or cynomolgus IL-36γ starting at 200 nM, followed by a 2-fold dilution. In some conditions this was performed in the presence of 200 nM m/h 144D464A, 200 nM m/h 144L124B, 200 nM m/h 144L249B, or 200 nM of human IL-36Ra. Assay plates were incubated at 5% CO2, 37° C. for 20 hours. Culture supernatant was then harvested and diluted 10-fold for measurement of IL-8 with R&D DuoSet IL-8 ELISA kit (Cat# DY208, R&D Systems, Minneapolis, Minnesota) as per manufacturer's instructions. To accommodate a 384-well screening format ELISA kit reagents were used at 15 μL volumes. O.D. 450 values from the IL-8 ELISA were graphed using GraphPad PRISM™ software. A reduction in O.D.450 values was interpreted as an illustration of antagonist activity. Cynomolgus keratinocytes stimulated with a titration of cynomolgus IL-36α or cynomolgus IL-36γ secrete IL-8 in a dose dependent manner, as measured by the O.D 450 values in an IL-8 ELISA. The amount of secreted IL-8 was dramatically reduced when the stimulation was performed in the presence of IL-36α and IL-36γ dual-antagonist monoclonal antibodies, or human IL-36Ra (see FIG. 6A to 6B).

This data confirmed that the IL-36α and IL-36γ dual-antagonist antibodies can antagonize cynomolgus IL-36α and cynomolgus IL-36γ stimulation of primary cynomolgus cells.

### 6.4 Example 4—Mechanism of Action Studies of IL-36α and IL-36γ Dual-Antagonist Monoclonal Antibodies

6.4.1 Simultaneous Inhibition of Human IL-36α and IL-36γ

A modified HaCaT assay was utilized to demonstrate the ability of IL-36α and IL-36γ dual antagonist monoclonal antibodies to simultaneously antagonize both IL-36α and IL-36γ. HaCaT cells were stimulated with a matrix of IL-36α and IL-36γ concentrations which were titrated 2-fold from left-to-right or top-to-bottom, respectively. The concentration of a given cytokine ranged from 50 nM to 0.39 nM, for a total of 8 different concentrations. HaCaT cells were stimulated with this matrix of cytokine in the presence of a range of chimeric mouse/human (m/h) 144D464A antibody (150 nM, 100 nM, 50 nM, 25 nM, 1 nM, 0 nM) or IL-36Ra (500 nM, 250 nM, 100 nM, 50 nM, 10 nM, 0 nM). Assay plates were incubated at 5% CO₂, 37° C. for 20 hours. Assay culture supernatant was then collected, diluted 5-fold, and secreted IL-8 was measured. A reduction in O.D.450 values was interpreted as an illustration of antagonist activity. The O.D. values are depicted in a greyscale heat map with higher O.D. values (i.e., higher IL-8 levels) corresponding to a darker color.

Chimeric monoclonal antibody m/h 144D464A was used as a representative IL-36α and IL-36γ dual-antagonist monoclonal antibody. With this assay we could observe the ability of m/h 144D464A to inhibit IL-8 secretion from cells stimulated simultaneously with IL-36α and IL-36γ. IL-36Ra could also suppress IL-8 secretion for cells stimulated simultaneously with IL-36α and IL-36γ, though higher concentrations of IL-36Ra were required to demonstrate antagonist activity similar to that observed for m/h 144D464A (see FIG. 7).

### 6.4.2 IL-36α and IL-36γ Dual Antagonist Monoclonal Antibodies Do Not Interfere With IL-36β Signaling

A modified HaCaT assay was utilized to determine if IL-36α and IL-36γ dual-antagonist antibodies, alone or pre-complexed with IL-36α or IL-36γ, impacted IL-36β signaling activity. HaCaT cells were stimulated with a titration human IL-36β starting at 200 nM, followed by a 2-fold dilution. In some conditions this was performed in the presence of 300 nM of m/h 144D464A, 300 nM of m/h 144D464A that had been pre-incubated with 25 nM of IL-36α, 300 nM of m/h 144D464A that had been pre-incubated with 50 nM of IL-36γ, or 300 nM of IL-36Ra. Controls included HaCaT cells cultured in the presence of 25 nM IL-36α, 25 nM IL-36α plus 300 nM m/h 144D464A, 50 nM IL-36γ, or 50 nM of IL-36γ plus 300 nM m/h 144D464A. Assay plates were incubated at 5% CO₂, 37° C. for 20 hours. Assay culture supernatant was then collected, diluted 5-fold, and secreted IL-8 was measured. A reduction in O.D. 450 values was interpreted as an illustration of antagonist activity.

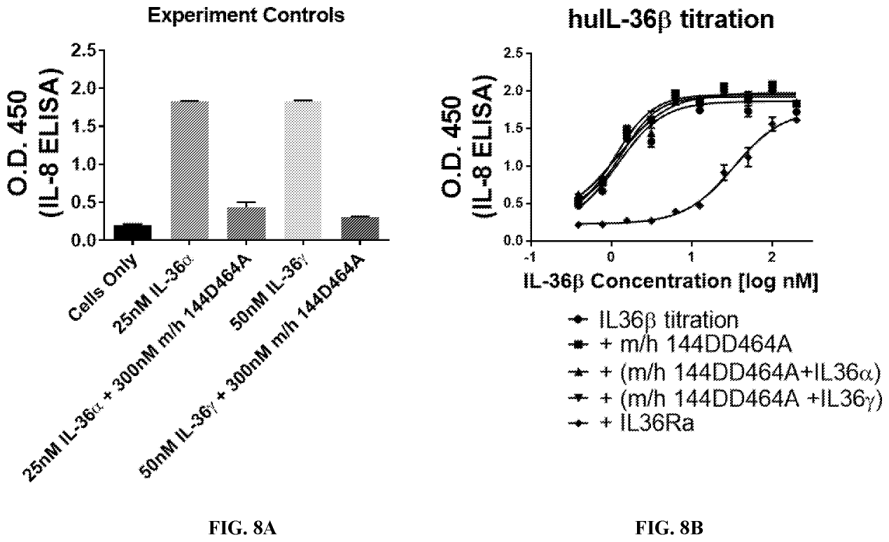

Chimeric monoclonal antibody m/h 144D464A was used as a representative IL-36α and IL-36γ dual-antagonist monoclonal antibody in this assay. Experimental controls demonstrated that 25 nM of IL-36α, or 50 nM or IL-36γ, could stimulate IL-8 production in HaCaT cells. When the IL-36α and IL-36γ were pre-incubated with 300 nM of m/h 144D464A, IL-8 secretion was inhibited. This demonstrated that these pre-formed antibody-cytokine complexes do not activate the IL-36 receptor (FIG. 8A). When HaCaT cells were stimulated with a titration of IL-36β, IL-8 was secreted in a dose dependent manner. Addition of 300 nM of IL-36Ra to the culture conditions could inhibit the amount of IL-8 secreted. However, neither m/h 144D464A, m/h 144D464A pre-complexed with IL-36α, nor m/h 144D464A pre-complexed with IL-36γ could inhibit IL-8 secretion induced by IL-36β. These results indicate that IL-36α and IL-36γ dual-antagonist antibodies, alone or pre-complexed with IL-36α or IL-36γ, do not impact IL-36β signaling activity (FIG. 8B).

### 6.4.3 IL-36α and IL-36γ Dual Antagonist Monoclonal Antibodies Can Cooperate With IL-36Ra to Inhibit Activity of IL-36α, IL-36β, and IL-36γ

A modified HaCaT assay was utilized to demonstrate that IL-36α and IL-36γ dual-antagonist mAbs do not interfere with IL-36Ra antagonist activity, and that they can cooperate with IL-36Ra to suppress IL-36α, IL-36β, and IL-36γ. HaCaT cells were stimulated with individual titrations of human IL-36α, IL-36β, and IL-36γ, or a combination of all 3. IL-36α titrations started at 7.5 nM, followed by a 2-fold dilution series. IL-36β titrations started at 1.875 nM, followed by a 2-fold dilution series. IL-36γ titrations started at 30 nM, followed by a 2-fold dilution series. In some instances this titration was done in the presence of 100 nM of m/h 144D464A, 100 nM of IL-36Ra, or a combination of 100 nM m/h 144D464A and 100 nM IL-36Ra. Assay plates were incubated at 5% CO₂, 37° C. for 20 hours. Assay culture supernatant was then collected and diluted 5-fold, and secreted IL-8 was measured. A reduction in O.D. 450 values was interpreted as an illustration of antagonist activity.

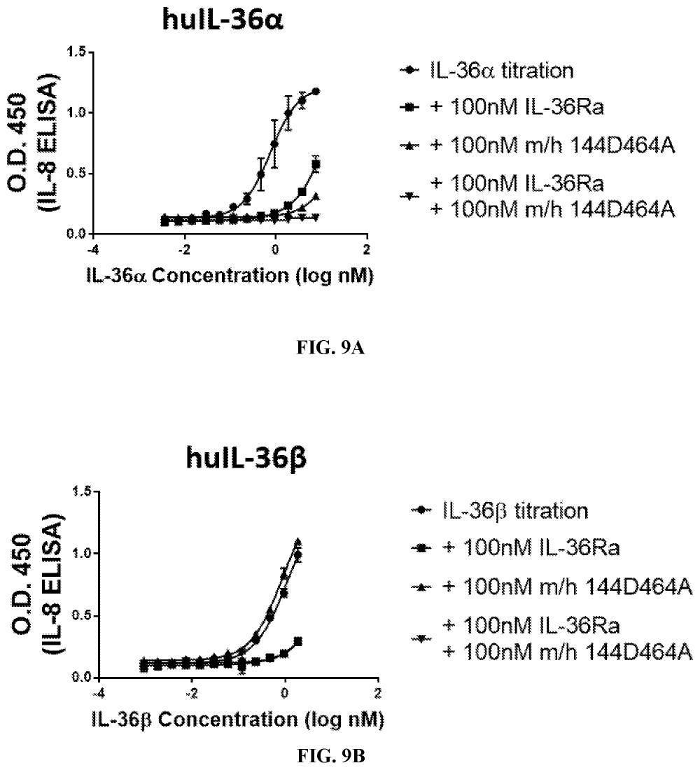

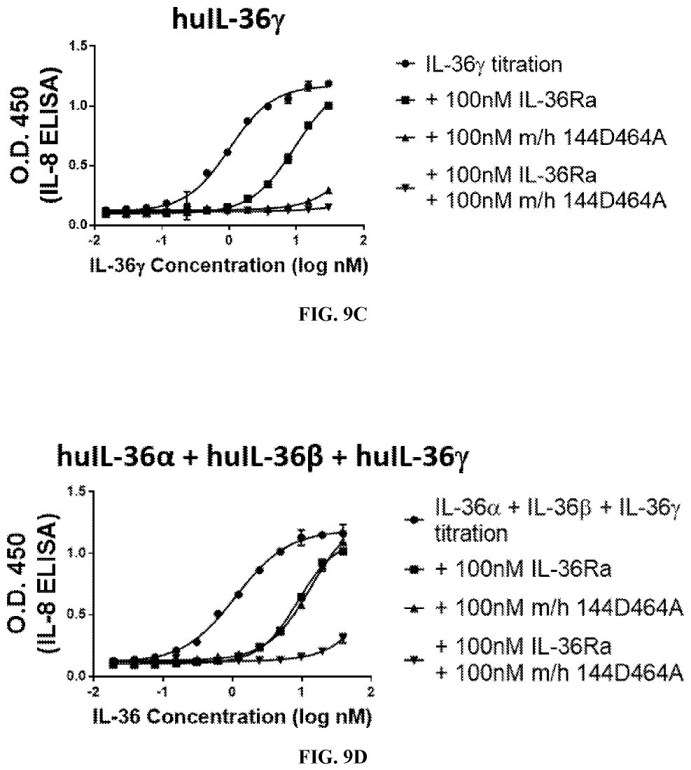

Chimeric monoclonal antibody m/h 144D464A was used as a representative IL-36α and IL-36γ dual-antagonist monoclonal antibody in this assay. When HaCaT cells were stimulated with a titration of IL-36α, IL-8 was secreted in a dose-dependent manner. When this stimulation was done in the presence of IL-36Ra, m/h 144D464A, or a mixture of both IL-36Ra and m/h 144D464A, IL-8 secretion was inhibited. Inhibitory activity could be ranked as follows: IL-36Ra+m/h 144D464A>m/h 144D464A>IL-36Ra (FIG. 9A). When HaCaT cells were stimulated with a titration of IL-36β, IL-8 was secreted in a dose-dependent manner. When this stimulation was done in the presence of m/h 144D464A, no inhibition of IL-8 was observed. When the stimulation was done in the presence of IL-36Ra or IL-36Ra+m/h 144D464A, IL-8 secretion was inhibited and the extent of inhibition between the two conditions was equal (FIG. 9B). This data indicates that only IL-36Ra can inhibit IL-36(3 activity, and that m/h 144D464A does not interfere with the antagonist activity of IL-36Ra.

When HaCaT cells were stimulated with a titration of IL-36γ, IL-8 was secreted in a dose-dependent manner. When this stimulation was done in the presence of IL-36Ra, m/h 144D464A, or a mixture of both IL-36Ra and m/h 144D464A, IL-8 secretion was inhibited. Inhibitory activity could be ranked as follows: IL-36Ra+m/h 144D464A>m/h 144D464A>>IL-36Ra (FIG. 9C). When HaCaT cells were stimulated with a combined titration of IL-36α+IL-36β +IL-36γ, IL-8 was secreted in a dose-dependent manner. When this stimulation was done in the presence of IL-36Ra, m/h 144D464A, or a mixture of both IL-36Ra and m/h 144D464A, IL-8 secretion was inhibited. Inhibitory activity could be ranked as follows: IL-36Ra+m/h 144D464A>>m/h 144D464A=IL-36Ra (FIG. 9D). This indicates that IL-36α and IL-36γ dual-antagonist monoclonal antibodies can cooperate with IL-36Ra to more potently inhibit all IL-36 receptor agonists.

### 6.5 Example 5—Antibody Affinity Assay

Antibodies were further tested for their binding to recombinant human or cynomolgus IL36α, IL-36γ in Biacore assays. In order to kinetically analyze the binding activity of the IL-36α and IL-36γ dual antagonist antibodies, the binding activity to human and cynomolgus IL-36α and IL-36γ was measured by surface plasmon resonance method (SPR). All of the following manipulations were carried out using a Biacore T200 (GE Healthcare Life Sciences, Pittsburgh, Pa.).

To determine antibody affinity to human and cynomolgus IL36, recombinant antibodies were immobilized on a CM5 sensor chip (cat# BR100012, GE Healthcare Life Sciences, Pittsburgh, Pa.) by an amine coupling chemistry. In particular, the kinetic assay was carried out by immobilizing on the chip approximately 2000 RU of recombinant antibody. Thereafter, recombinant human or cynomolgus IL36 proteins, serially diluted from a high concentration, were allowed to run at a flow rate of 30 μL/min onto the chip for 420 seconds. The dissociation time was 3600 seconds and the binding curves were measured at 25° C. Regeneration was performed with 10 mM glycine pH 1.5 for 30s. Alternatively, the analytes were injected with increasing concentrations in a single cycle, the surface not being regenerated between injections, using a method referred to as “single cycle kinetics.” In particular, either anti-human or anti-mouse Fc specific antibodies were immobilized on a CM5 sensor chip (cat# BR100012, GE Healthcare Life Sciences, Pittsburgh, Pa.) by an amine coupling chemistry. Thereafter, IL-36α and IL-36γ dual antagonist mouse/human chimeric or mouse antibodies were captured at approximately 200 RUs, followed by injections of increasing concentrations of recombinant human and cynomolgus IL36 proteins. The association time was 300 seconds, and the final dissociation time was 1200 seconds. At the end of the analyte injections, the surface was regenerated with either 3M MgCl₂ (anti-human Fc capture antibody) for 30 s or 10 mM glycine-HCl pH 1.7 for 180 s (anti-mouse IgG capture antibody).

The raw data were double referenced by subtraction of the signals from a reference flow cell without immobilized ligand and a buffer blank. The sensorgram corresponding to each concentration was obtained. The analysis was carried out using a 1:1 Langmuir fit model, using the analysis software attached to the apparatus, Biacore T200 Evaluation software, thereby calculating an association rate constant kₐ [M⁻¹s⁻¹] and a dissociation rate constant k_{d}[s⁻¹] for the recombinant IL36 proteins.

As a result of using a 1:1 Langmuir fit model, an equilibrium dissociation constant KD (k_{d}/kₐ) of exemplary antibodies was determined (see Tables 15-16 below).

TABLE 15
Binding kinetics of IL-36α and IL-36γ dual-antagonist
antibodies to human and cynomolgus IL-36α

| Clone | Analyte | k_{d} (1/s) | kₐ (1/Ms) | K_{D} (nM) |
|---|---|---|---|---|
| 144D464A | human IL-36 alpha_R12 | 3.09E−04 | 1.22E+05 | 2.53 |
| | cynomolgus IL-36 alpha | n.t | n.t | n.t |
| m/h | human IL-36 alpha_Q12 | 3.91E−04 | 3.61E+05 | 1.082 |
| 144D464A | human IL-36 alpha_R12 | 3.09E−04 | 1.42E+05 | 2.178 |
| | cynomolgus IL-36 alpha | 2.67E−04 | 1.63E+05 | 1.635 |
| 144L249B | human IL-36 alpha_R12 | 8.99E−04 | 5.39E+05 | 1.666 |
| | cynomolgus IL-36 alpha | 8.39E−04 | 6.58E+05 | 1.276 |
| m/h | human IL-36 alpha_Q12 | 8.25E−04 | 8.05E+05 | 1.024 |
| 144L249B | human IL-36 alpha_R12 | n.t | n.t | n.t |
| | cynomolgus IL-36 alpha | n.t | n.t | n.t |
| 144L124B | human IL-36 alpha_R12 | 416.6E−04 | 116.9E+05 | 3.565 |
| | cynomolgus IL-36 alpha | 192.1E−04 | 75.6E+05 | 2.541 |
| m/h | human IL-36 alpha_Q12 | 226.9E−04 | 122.1E+05 | 1.859 |
| 144L124B | human IL-36 alpha_R12 | n.t | n.t | n.t |
| | cynomolgus IL-36 alpha | n.t | n.t | n.t |
| 144D666C | human IL-36 alpha_R12 | 18.36E−04 | 1.02E+05 | 17.98 |
| | cynomolgus IL-36 alpha | n.t | n.t | n.t |
| m/h | human IL-36 alpha_Q12 | 8.34E−04 | 4.76E+05 | 1.753 |
| 144D666C | human IL-36 alpha_R12 | 21.92E−04 | 0.8728E+05 | 25.11 |
| | cynomolgus IL-36 alpha | n.t | n.t | n.t |
| m/h | human IL-36 alpha_Q12 | 86.82E−04 | 12.39E+05 | 7.007 |
| 144J171G | human IL-36 alpha_R12 | n.t | n.t | n.t |
| | cynomolgus IL-36 alpha | n.t | n.t | n.t |

Note:
n.t. means not tested

TABLE 16
Binding kinetics of IL-36α and IL-36γ dual-antagonist
antibodies to human and cynomolgus IL-36γ

| Clone | Analyte | k_{d} (1/s) | kₐ (1/Ms) | K_{D} (nM) |
|---|---|---|---|---|
| 144D464A | human IL-36 gamma | 3.20E−04 | 0.8396E+05 | 3.81 |
| | cynomolgus IL-36 gamma | n.t | n.t | n.t |
| m/h | human IL-36 gamma | 2.18E−04 | 1.47E+05 | 1.483 |
| 144D464A | cynomolgus IL-36 gamma | 3.42E−04 | 1.86E+05 | 1.842 |
| 144L249B | human IL-36 gamma | 1.94E−04 | 5.16E+05 | 0.3767 |
| | cynomolgus IL-36 gamma | 1.97E−04 | 6.31E+05 | 0.3125 |
| 144L124B | human IL-36 gamma | 6.91E−04 | 8.29E+05 | 0.833 |
| | cynomolgus IL-36 gamma | 4.88E−04 | 5.93E+05 | 0.8237 |
| 144L472A | human IL-36 gamma | 20.83E−04 | 0.02072E+05 | 1000 |
| | cynomolgus IL-36 gamma | n.t | n.t | n.t |
| 144D666C | human IL-36 gamma | 6.76E−04 | 6.25E+05 | 1.081 |
| | cynomolgus IL-36 gamma | n.t | n.t | n.t |
| m/h | human IL-36 gamma | 3.42E−04 | 1.12E+05 | 3.062 |
| 144D666C | cynomolgus IL-36 gamma | n.t | n.t | n.t |

Note:
n.t. means not tested

### 6.6 Example 6—Preparation of Humanized Antibodies

### 6.6.1 Design of VL and VH of humanized 144D464A Antibody

First, FR amino acid sequences of VL of a human antibody suitable for transplantation of the CDRs of 144D464A VL were selected in the following manner.

Human antibody sequences having high homology with VL of 144D464A were searched using BLASTP database provided by The National Center for Biotechnology Information. As a result, the human antibody sequence of GeneBank ID AAA59034.1 exhibited the highest homology with 144D464A, and thus the FR of this antibody was selected. LV0 (SEQ ID NO: 114) was designed by transplanting the amino acid sequences of the CDR L1, the CDR L2 and the CDR L3 of 144D464A (SEQ ID NOs: 83, 84 and 85, respectively) into suitable positions in the human antibody FR sequences.

Next, FR amino acid sequences of VH of a human antibody suitable for transplantation of the CDRs of 144D464A VH were selected in the same manner, human antibody sequences having high homology with VH of 144D464A were searched using BLASTP database. The human antibody sequence of GeneBank ID CAB45243.1 exhibited the highest homology with 144D464A, so the FR of this antibody was selected. HVO (SEQ ID NO: 115) was designed by transplanting the amino acid sequences of the CDR H1, the CDR H2 and the CDR H3 of 144D464A (SEQ ID NOs: 68, 69 and 70, respectively) into suitable positions in the human antibody FR sequences.

To avoid decrease of binding activity caused by humanization, amino acid residues in the FR, which are different between the human antibody and rodent-derived antibody and considered to affect the binding activity, can be substituted.

Amino acid residues in LV0 and HV0 which were different from those of 144D464A were selected. Further, amino acids which are expected to affect the binding activity were identified by comparing three dimensional structure using MOE (MOLSIS). As a result, the amino acids that could affect the binding activity were selected from a goup including Pro 8, Val 12, Phe 38, Gln 40, Ala 45, Pro 46, Arg 47, Thr 48, Ser 51, Trp 59, Thr 60, Leu 77 and Asp 87 in the amino acid sequence of LVO, and Gln 1, Lys 12, Val 20, Tyr 27, Thr 28, Phe 29, Thr 30, Arg 38, Met 48, Arg 67, Val 68, Ala 72, Ser 77, Ala 79, Met 81, Leu 83 and Val 117 in the amino acid sequence of HV0.

The VL and VH of a humanized antibody comprising various modifications were designed wherein at least one amino acid residue among the above selected amino acid residues is substituted with the one present in the same position of 144D464A antibody. Specifically, in the case of the VL, at least one substitution selected from Pro 8 with Ser, Val 12 with Thr, Phe 38 with Val, Gln 40 with Glu, Ala 45 with Leu, Pro 46 with Phe, Arg 47 with Ala, Thr 48 with Gly, Ser 51 with Gly, Trp 59 with Gly, Thr 60 with Val, Leu 77 with Ile and Asp 87 with Ile were introduced. In the case of the VH, at least one substitution selected from Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ala 72 with Thr, Ser 77 with Asp, Ala 79 with Val, Met 81 with Leu, Leu 83 with Phe and Val 117 with Leu were introduced.

By modifying LVO (SEQ ID NO: 114) in the above-mentioned method, the following VL regions were designed: LV3a (SEQ ID NO: 116), LV3b (SEQ ID NO: 117), LV4a (SEQ ID NO: 118), LV4b (SEQ ID NO: 119), LV5a (SEQ ID NO: 120), LV5b (SEQ ID NO: 121), LV5c (SEQ ID NO: 122), LV5d (SEQ ID NO: 123), LV5e (SEQ ID NO: 124), LV6a (SEQ ID NO: 125), LV6b (SEQ ID NO: 126), LV6c (SEQ ID NO: 127), LV6d (SEQ ID NO: 128), LV6e (SEQ ID NO: 129), LV7a (SEQ ID NO: 130), LV7b (SEQ ID NO: 131), LV8 (SEQ ID NO: 132), LV9 (SEQ ID NO: 133), LV11 (SEQ ID NO: 134), LV12(+1) (SEQ ID NO: 135), LV9are (SEQ ID NO: 136), LV10re (SEQ ID NO: 137) and LV11re (SEQ ID NO: 138) (see FIG.10).

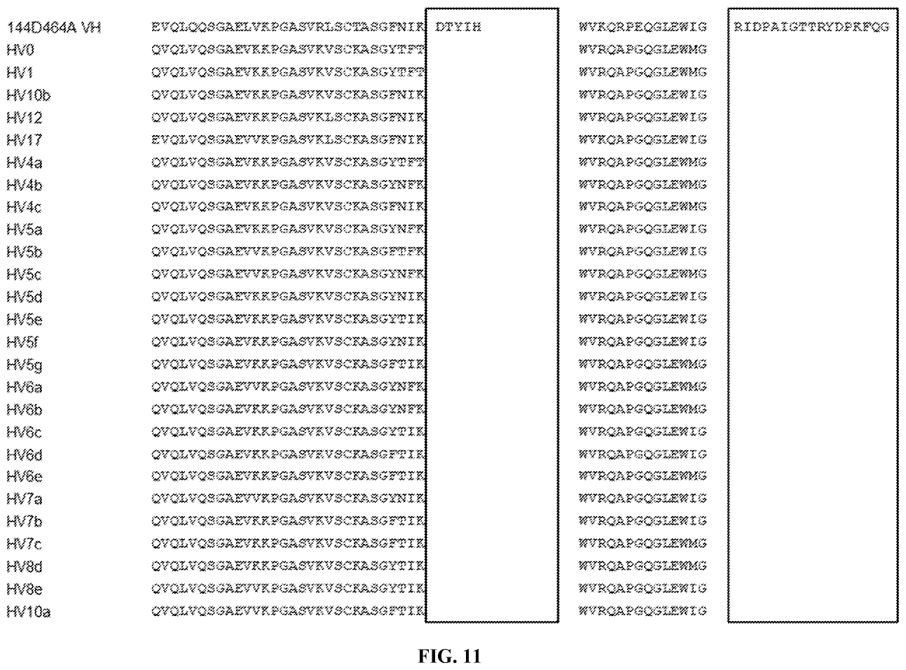

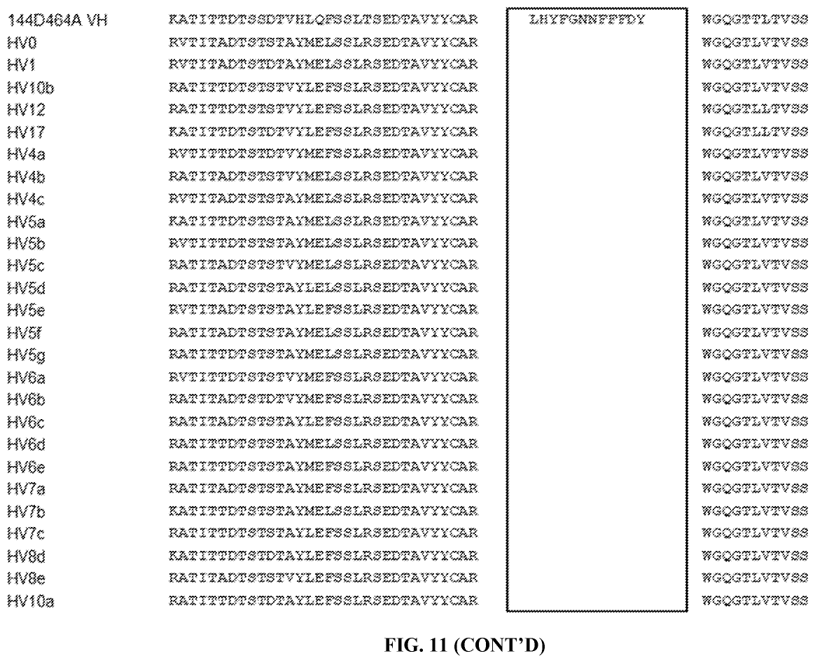

By modifying HVO (SEQ ID NO: 115) in the above-mentioned method, the following VH regions were designed: HV1 (SEQ ID NO: 139), HV4a (SEQ ID NO: 140), HV4b (SEQ ID NO: 141), HV4c (SEQ ID NO: 142), HV5a (SEQ ID NO: 143), HV5b (SEQ ID NO: 144), HV5c (SEQ ID NO: 145), HV5d (SEQ ID NO: 146), HV5e (SEQ ID NO: 147), HV5f (SEQ ID NO: 148), HV5g (SEQ ID NO: 149), HV6a (SEQ ID NO: 150), HV6b (SEQ ID NO: 151), HV6c (SEQ ID NO: 152), HV6d (SEQ ID NO: 153), HV6e (SEQ ID NO: 154), HV7a (SEQ ID NO: 155), HV7b (SEQ ID NO: 156), HV7c (SEQ ID NO: 157), HV8d (SEQ ID NO: 158), HV8e (SEQ ID NO: 159), HV10a (SEQ ID NO: 160), HV10b (SEQ ID NO: 161), HV12 (SEQ ID NO: 162) and HV17 (SEQ ID NO: 163) (see FIG. 11).

Humanized 144D464A antibodies comprising the above described variable regions are represented by the combination of the names of VL and VH. For example, the humanized 144D464A antibodies comprising LV7a and HV10b; LV9are and HV10b; LV10re and HV10b; and LV11re and HV10b are referred to as 144D464A LV7a HV10b, 144D464A LV9are HV10b, 144D464A LV10re HV10b and 144D464A LV11re HV10b, respectfively.

### 6.6.2 Design of VL and VH of Humanized 144L249B

Amino acid sequences of VL of humanized 144L249B antibodies were designed by transplanting the amino acid sequences of the CDR L1 and the CDR L3 of 144L249B (SEQ ID NOs: 86 and 88, respectively), and the CDR L2 of 144D464A (SEQ ID NO: 84), one amino acid residue of which is different from the CDR L2 of 144L249B (SEQ ID NO: 87), into the FR amino acid sequences of the human antibody sequence of GeneBank ID AAA59034.1, using the method described above in Section 6.6.1. The CDRL2 of 144D464A was used instead of that of 144L249B to reduce immunogenicity. Similarly, amino acid sequences of VH of humanized 144L249B antibodies were designed by transplanting the amino acid sequences of the CDR H1, the CDR H2 and the CDR H3 of 144L249B (SEQ ID NOs: 71, 69 and 72, respectively) into the FR amino acid sequences of the human antibody sequence of GeneBank ID CAB45243.1. The resulted humanized VL and VH are named as LVO (SEQ ID NO: 164) and HVO (SEQ ID NO: 165), respectively.

The VL and VH of a humanized antibody comprising various amino acid modifications were designed in the same way as in Section 6.6.1. Specifically, in the case of VL, at least one substitution selected from Pro 8 with Ser, Val 12 with Thr, Phe 38 with Val, Gln 40 with Glu, Ala 45 with Leu, Pro 46 with Phe, Arg 47 with Thr, Thr 48 with Gly, Ser 51 with Gly, Trp 59 with Gly, Thr 60 with Val, Leu 77 with Ile and Asp 87 with Ile were introduced. In the case of VH, at least one substitution selected from Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ile 70 with Leu, Ala 72 with Thr, Ser 77 with Asn, Met 81 with Leu and Val 117 with Leu were introduced.

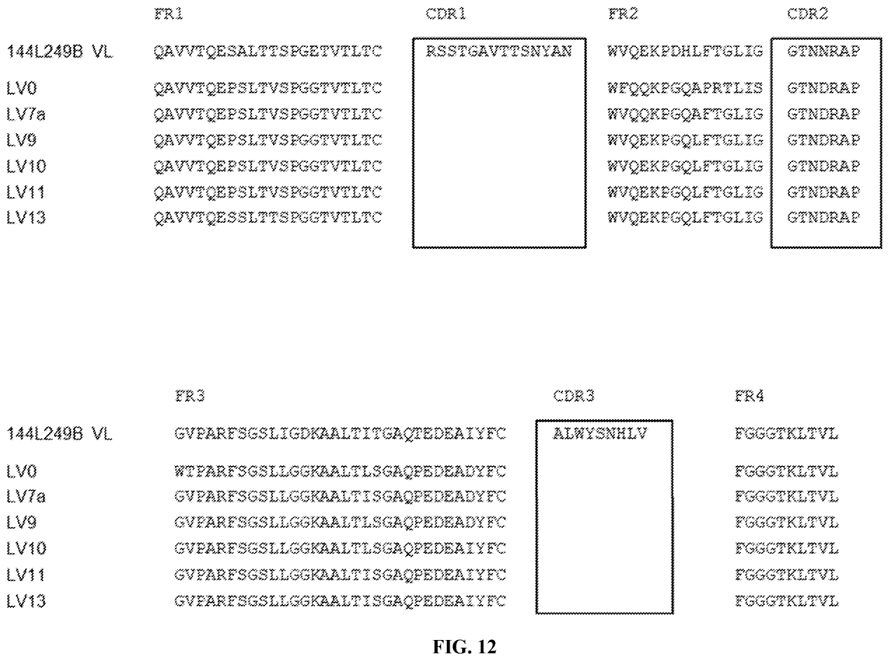

By modifying LVO (SEQ ID NO: 164), the following VL regions were designed: LV7a (SEQ ID NO: 166), LV9 (SEQ ID NO: 167), LV10 (SEQ ID NO: 168), LV11 (SEQ ID NO: 169) and LV13 (SEQ ID NO: 170) (see FIG.12).

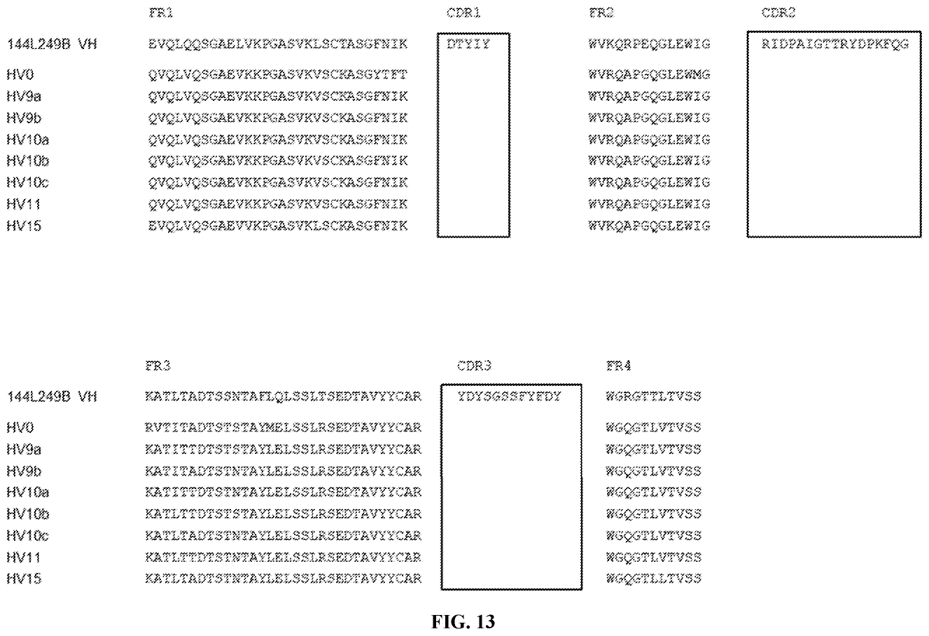

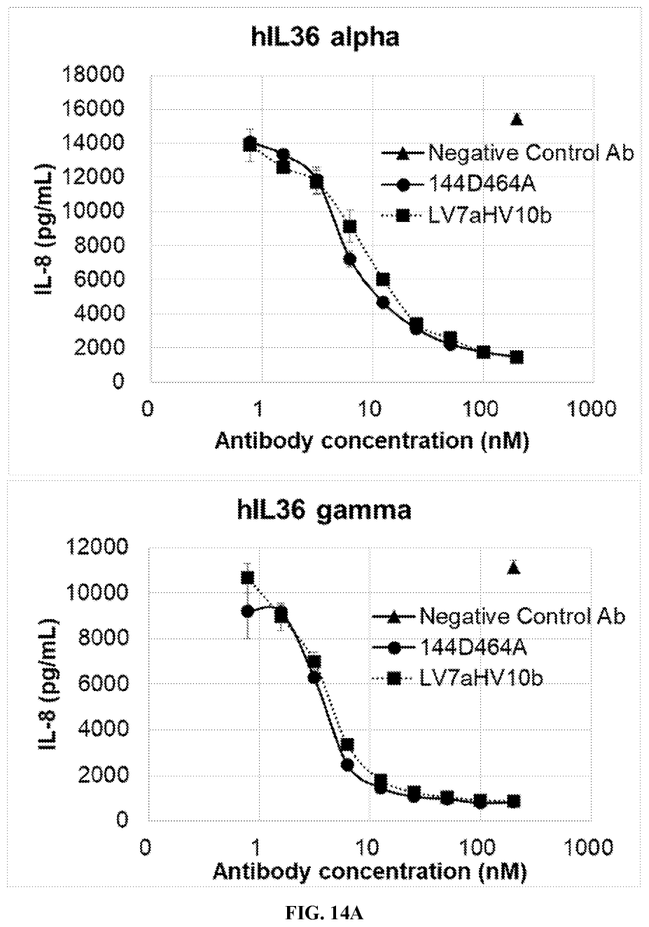

By modifying HVO (SEQ ID NO: 165), the following VH regions were designed: HV9a (SEQ ID NO: 171), HV9b (SEQ ID NO: 172), HV10a (SEQ ID NO: 173), HV10b (SEQ ID NO: 174), HV10c (SEQ ID NO: 175), HV11 (SEQ ID NO: 176) and HV15 (SEQ ID NO: 177) (see FIG.13).

A humanized 144L249B antibody comprising the above described variable regions are represented by the combination of the names of VL and VH. For example, the humanized 144L249B antibodies comprising LV7a and HV11; LV9 and HV11; LV9 and HV10b; and LV9 and HV10c are referred as 144L249B LV7a HV11, 144L249B LV9 HV11, 144L249B LV9 HV10b and 144L249B LV9 HV10c, respectively.

### 6.6.3 Design of Nucleic Acid Sequence of Humanized Antibody

The nucleic acid sequences which encode amino acid sequences of humanized antibodies were designed using codons highly frequently used in animal cells. With these sequences, construction of humanized antibody expression vectors described below and expression of the corresponding antibodies were carried out.

### 6.6.4 Construction of Humanized 144D464A and Humanized 144L249B Expression Vectors

DNA fragments of the variable regions were entirely synthesized. The DNA fragments of each VL were inserted into a pCI based transient expression vector which has a gene coding human lambda light chain constant region. The DNA fragments of each VH were inserted into a pCI based transient expression vector which has a gene coding human heavy chain constant region. E. coli DH5a competent cells (Takara) were transformed using the prepared vectors, and then a large amount of plasmids were prepared for further experiments.

### 6.6.5 Transient Expression of Humanized 144D464A and Humanized 144L249B Antibodies

Transient expression of humanized antibodies was performed using Expi293F expression system (ThermoScientific) in the same way as described above. The mammalian expression vectors for antibody light chain and antibody heavy chain were mixed with each other at a ratio of 1:2.

### 6.6.6 Purification of Humanized 144D464A and Humanized 144L249B Antibodies

The humanized antibodies were purified by affinity purification using MabSelect SuRe (GE Healthcare). After the resin was equilibrated with PBS, the culture supernatant was loaded and washed twice with PBS.

After the washing, the antibody was eluted using an elution buffer (20 mM citric acid, 50 mM NaCl, pH 3.4) and one-tenth the total amount of a neutralization buffer (1 mol/L phosphoric acid, pH 7.0) was added. Next, buffer substitution with PBS by NAP25 (GE Healthcare) was performed. The resultant was concentrated by ultrafiltration using Amicon Ultra Centrifugal Filter Units (Millipore) and the absorbance at 280 nm was measured using Nanodrop8000 for concentration determination.

### 6.7 Example 7—Affinity and Neutralization Activity of Humanized Antibodies

The humanized antibodies obtained in Example 6 were tested for their binding to recombinant human or cynomolgus macaque IL36a and IL-36γ in Biacore assays. The association time was set to 60 seconds, and the final dissociation time was set to 600 seconds. Equilibrium dissociation constant K_{D} (kd/ka) of exemplary antibodies was determined using a 1:1 Langmuir fit model (see Tables 17-18 below).

TABLE 17
Binding kinetics of humanized 144D464A to human and
cynomolgus macaque IL-36α and IL-36γ

| Clone | Analyte | k_{d} (1/s) | kₐ (1/Ms) | K_{D} (M) |
|---|---|---|---|---|
| 144D464A | hIL-36alpha | 2.74E+5 | 36.15E−5 | 13.19E−10 |
| | hIL-36gamma | 3.88E+5 | 34.05E−5 | 8.77E−10 |
| | cyIL-36alpha | 4.65E+5 | 33.85E−5 | 7.27E−10 |
| | cyIL-36gamma | 4.93E+5 | 42.66E−5 | 8.65E−10 |
| LV7a | hIL-36alpha | 2.19E+5 | 45.61E−5 | 20.88E−10 |
| HV10b | hIL-36gamma | 3.43E+5 | 45.08E−5 | 13.15E−10 |
| | cyIL-36alpha | 3.62E+5 | 45.12E−5 | 12.47E−10 |
| | cyIL-36gamma | 4.43E+5 | 52.29E−5 | 11.81E−10 |
| LV9are | hIL-36alpha | 2.12E+5 | 44.53E−5 | 21.04E−10 |
| HV10b | hIL-36gamma | 3.36E+5 | 43.63E−5 | 13.01E−10 |
| | cyIL-36alpha | 3.54E+5 | 42.24E−5 | 11.93E−10 |
| | cyIL-36gamma | 4.34E+5 | 49.27E−5 | 11.34E−10 |
| LV10re | hIL-36alpha | 2.29E+5 | 42.94E−5 | 18.73E−10 |
| HV10b | hIL-36gamma | 3.53E+5 | 42.47E−5 | 12.04E−10 |
| | cyIL-36alpha | 3.72E+5 | 41.53E−5 | 11.16E−10 |
| | cyIL-36gamma | 4.60E+5 | 47.02E−5 | 10.23E−10 |
| LV11re | hIL-36alpha | 2.26E+5 | 42.18E−5 | 18.64E−10 |
| HV10b | hIL-36gamma | 3.46E+5 | 42.00E−5 | 12.13E−10 |
| | cyIL-36alpha | 3.69E+5 | 40.74E−5 | 11.04E−10 |
| | cyIL-36gamma | 4.46E+5 | 46.35E−5 | 10.40E−10 |

TABLE 18
Binding kinetics of humanized 144L249B to human and
cynomolgus macaque IL-36α and IL-36γ

| Clone | Analyte | k_{d} (1/s) | kₐ (1/Ms) | K_{D} (M) |
|---|---|---|---|---|
| 144L249B | hIL-36alpha | 5.15E+5 | 7.85E−4 | 15.30E−10 |
| | hIL-36gamma | 9.90E+5 | 3.95E−4 | 3.99E−10 |
| | cyIL-36alpha | 9.05E+5 | 1.05E−3 | 11.60E−10 |
| | cyIL-36gamma | 11.32E+5 | 3.26E−4 | 2.88E−10 |
| LV7a HV11 | hIL-36alpha | 7.49E+5 | 10.30E−4 | 13.70E−10 |
| | hIL-36gamma | 8.59E+5 | 6.78E−4 | 7.89E−10 |
| | cyIL-36alpha | 13.98E+5 | 1.40E−3 | 10.00E−10 |
| | cyIL-36gamma | 9.13E+5 | 8.31E−4 | 9.10E−10 |
| LV9 HV11 | hIL-36alpha | 7.91E+5 | 10.20E−4 | 12.90E−10 |
| | hIL-36gamma | 8.88E+5 | 7.06E−4 | 7.95E−10 |
| | cyIL-36alpha | 14.60E+5 | 1.35E−3 | 9.27E−10 |
| | cyIL-36gamma | 9.48E+5 | 8.66E−4 | 9.13E−10 |
| LV9 HV10b | hIL-36alpha | 7.14E+5 | 9.89E−4 | 13.90E−10 |
| | hIL-36gamma | 7.72E+5 | 7.28E−4 | 9.42E−10 |
| | cyIL-36alpha | 13.38E+5 | 1.26E−3 | 9.44E−10 |
| | cyIL-36gamma | 8.55E+5 | 9.69E−4 | 11.32E−10 |
| LV9 HV10c | hIL-36alpha | 8.50E+5 | 8.43E−4 | 9.92E−10 |
| | hIL-36gamma | 9.99E+5 | 6.25E−4 | 6.25E−10 |
| | cyIL-36alpha | 15.28E+5 | 1.03E−3 | 6.74E−10 |
| | cyIL-36gamma | 10.93E+5 | 6.03E−4 | 5.52E−10 |

Neutralization activity of humanized 144D464A and humanized 144L249B antibodies obtained in Example 6 were analyzed. The humanized antibodies were evaluated for their antagonist activity in the HaCaT assay. 35 μL of each antibody diluted in HaCaT culture media [DMEM (cat#10313-021, Gibco), 10% FBS (cat#15140-163, Gibco), 2 mM L-glutamine (cat#25030-081, Gibco) and 1% PenStrep (cat#15140-160, Gibco)] were added to 96-well flat bottom plate (cat#167008, Nunc) for a final assay concentration starting at 300 nM, followed by a 2-fold dilution series. 35 μL of IL-36α (cat#6995-IL-010/CF, R&D Systems) or IL-36γ (cat#6835-IL-010/CF, R&D Systems) diluted in HaCaT culture media was added to the assay plate for a final assay concentration of 10 nM. The assay plate was left for 15 minutes at room temperature, then 35 μL of HaCaT cells (cat#TT0020001, AddexBio) suspended in HaCaT culture media was added to the assay plate for a final cell number of 35,000 cells/well. The assay plate was incubated at 5% CO₂, 37° C. for 24 hours. Culture supernatant was then harvested and the amount of IL-8 secreted in the supernatant was measured by AlphaLISA (cat#AL224C, PerkinElmer), and IC50 values were calculated using Excel XLFit software.

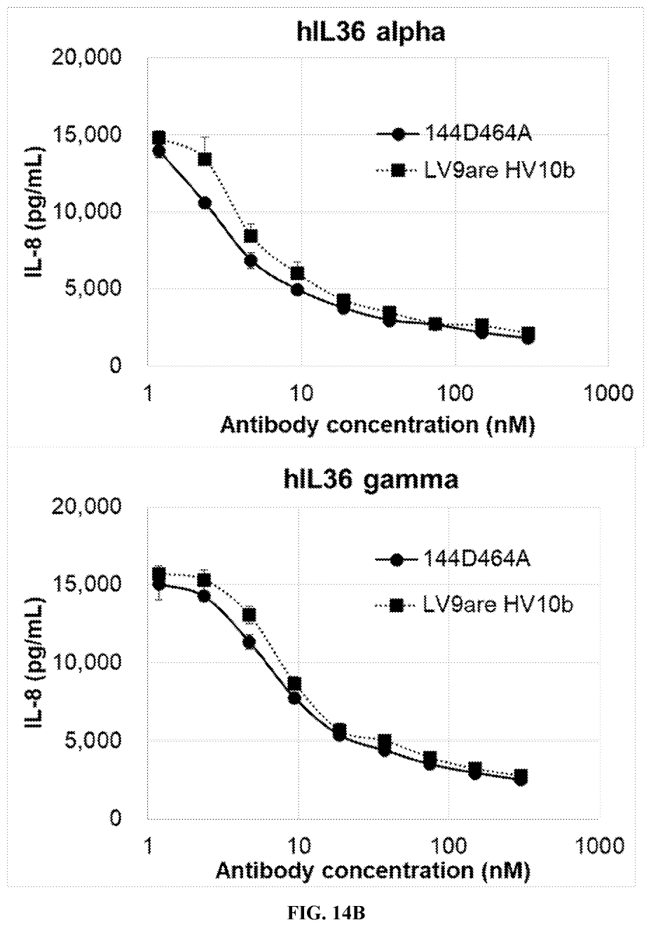

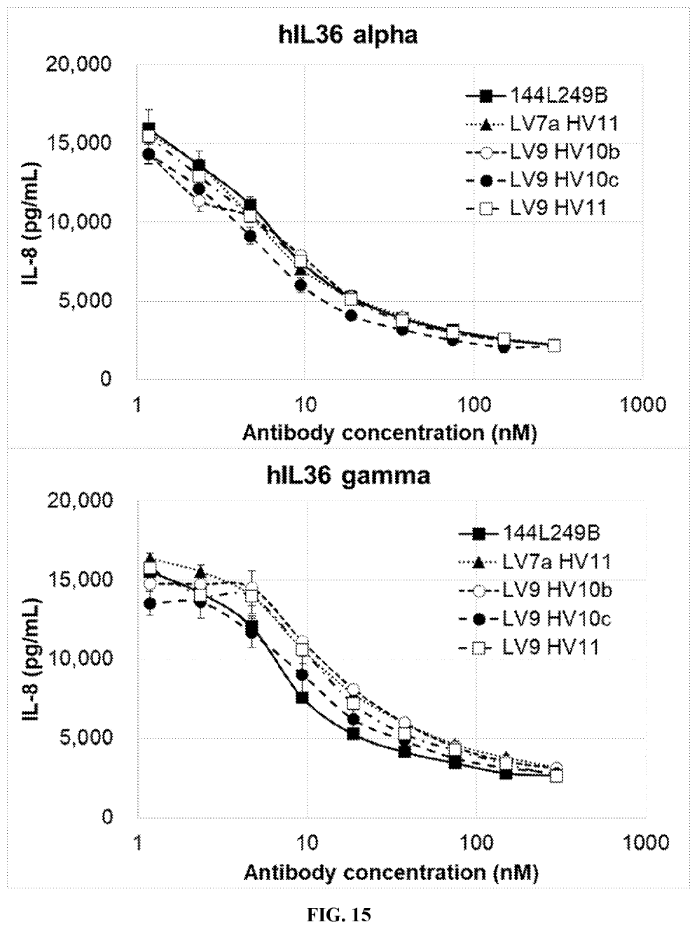

The humanized 144D464A antibodies and the humanized 144L249B antibodies antagonized both human IL-36α and IL-36γ (see FIGS. 14A, 14B and 15 and Tables 19 and 20).

TABLE 19
IC₅₀ values of humanized 144D464A antagonizing human IL-36α
and IL-36γ in HaCaT functional assays

| IC₅₀ | Human | Human |
|---|---|---|
| (nM) | IL-36alpha | IL-36gamma |
| LV7a HV10b | 3.90 | 7.83 |
| LV9are HV10b | 4.51 | 12.52 |

TABLE 20
IC₅₀ values of humanized 144L249B antagonizing human IL-36α
and IL-36γ in HaCaT functional assays

| IC₅₀ | Human | Human |
|---|---|---|
| (nM) | IL-36alpha | IL-36gamma |
| LV7a HV11 | 9.20 | 25.84 |
| LV9 HV10b | 6.61 | 20.68 |
| LV9 HV10c | 5.13 | 11.47 |
| LV9 HV11 | 7.68 | 16.54 |

### 6.8 Example 8—Crystallization of IL-36α-144L249BLV9HV10C Fab Complex and IL-36γ-144L249BLV9HV10C Fab Complex

For crystallization studies, purified humanized 144L249BLV9HV10c (L249B) Fab and slight molar excess of IL-36α and IL-36γ proteins were mixed separately and incubated at room temperature for 1 hr. The complex was then concentrated to 3 mg/ml and subjected to crystallization. Initial crystallization trails were performed at both 22° C. and 4° C. and tested over 800 different crystallization conditions (JCSG core+, 1-4, Wizard, MB suite and PEG ion screens) by sitting drop vapor diffusion method in a 96-well format using a nano-liter dispensing liquid handling robot (Art Robbins Phenix). Optimization of crystallization conditions was performed manually by both hanging drop and sitting drop methods by equilibrating 1.2 μl of protein (50 mM HEPES, pH 7.0 and 150 mM NaCl) and 0.8 μl of reservoir solution at 4° C.

Crystals of IL-36α—L249B Fab complex grew over 7 days in various conditions, however, crystals grown over 15 days at 4° C. by hanging drop method using the precipitant 2M ammonium sulfate, 0.2 M lithium sulfate and 0.1 M CAPS, pH 10.5 and in the presence of additive 30% sucrose generated high quality-diffraction. All crystals were flash-cooled in liquid nitrogen in their crystallization buffer containing 20% glycerol for subsequent data collection.

Crystals of IL-36γ—L249B Fab complex grew over 3 days in various conditions having PEG as a common precipitant. The crystals generated at 4° C. by sitting drop method in a well solution consisting of 0.2 M potassium citrate tribasic monohydrate and 20% WN PEG 3350 generated high quality-diffraction. Prior to diffraction, these crystals were cryoprotected by immersing the crystal in a mixture of paratone oil and paraffin oil (1:1 ratio) and were flash-cooled in liquid nitrogen for subsequent data collection.

Native X-ray diffraction data for the crystals of IL-36α—L249B Fab complex was collected remotely at Stanford Synchrotron Radiation Light Source (SSRL) beam line 14-1 using Dectris EIGER 16M detector at a wavelength of 1.19 Å and at 100 K temperature. The diffraction data images were collected with 0.1° oscillation and 5 sec exposure time. The data images were indexed, integrated and scaled in HKL 3000 package (see Kabsch, W. (2010) Integration, scaling, space-group assignment and post-refinement. Acta Crystallographica Section D: Biological Crystallography 66, 133-144) to an overall resolution of 2.7 Å. IL-36α—L249B Fab complex belong to the space group H3₂ with unit cell dimensions: a=148.0 Å, b=148.0 Å, c=410.7 Å, α=90°, β=90°, γ=90°.

The diffraction data for IL-36γ—L249B Fab complex was collected remotely at SSRL beamline 9-2 using a PILATUS 6M PAD detector at a wavelength of 0.97 Å and a temperature of 100 K. Data images were collected with 0.15° oscillation and 1-3 sec exposure time for different crystals. Multi crystal dataset was generated by merging four individual native datasets in AUTOPROC (Vonrhein, C., Flensburg, C., Keller, P., Sharff, A., Smart, O., Paciorek, W., Womack, T., and Bricogne, G. (2011) Data processing and analysis with the autoPROC toolbox. Acta Crystallographica Section D 67, 293-302) and STARANISO (Tickle, I. J., Flensburg, C., Keller, P., Paciorek, W., Sharff, A., and Vonrhein, C., Bricogne, G. (2018) STARANISO. Cambridge, United Kingdom: Global Phasing Ltd.). The data was processed to an overall resolution of 2.65 Å. IL-36γ—L249B Fab complex belong to the space group P 6₄ 2 2 with unit cell dimensions: a=112.9 Å, b=112.9 Å, c=199.7 Å, α=90°, β=90°, γ=120°.

The position of IL-36α in the asymmetric unit was determined by molecular replacement method PHASER-MR (see Rossmann, M. G. (1972) The Molecular Replacement Method, Gordon & Breach, New York; Vagin, A. A., and Teplyakov, A. (1997) MOLREP:an automated programm for molecular replacement. J. Appl. Cryst. 30, 1022-1025) using the previously reported IL-36γ structure (PDB 4IZE) as a search model. Similarly, the position of L249B Fab was also identified by molecular replacement method using the L and H chain of the mouse IgG1 Fab F124 (anti-hepatitis B surface antigen MAb, PDB ID 1F11) separately, as a starting search model. Starting with initial phases obtained by MR, the L249B Fab model and IL-36α were built gradually by cycles of iterative model building and manually built into the Fo-Fc electron density map using COOT function (see Emsley, P., and Cowtan, K. (2004) Coot: model-building tools for molecular graphics. Acta Crystallogr D Biol Crystallogr 60, 2126-2132; Emsley, P., Lohkamp, B., Scott, W. G., and Cowtan, K. (2010) Features and development of Coot. Acta crystallographica. Section D, Biological crystallography 66, 486-501) as part of the CCP4 suite (see Potterton, E., Briggs, P., Turkenburg, M., and Dodson, E. (2003) A graphical user interface to the CCP4 program suite. Acta Crystallogr. D59, 1131-1137; and Winn, M. D., Ballard, C. C., Cowtan, K. D., Dodson, E. J., Emsley, P., Evans, P. R., Keegan, R. M., Krissinel, E. B., Leslie, A. G., McCoy, A., McNicholas, S. J., Murshudov, G. N., Pannu, N. S., Potterton, E. A., Powell, H. R., Read, R. J., Vagin, A., and Wilson, K. S. (2011) Overview of the CCP4 suite and current developments. Acta crystallographica. Section D, Biological crystallography 67, 235-242). The models were further refined using PHENIX/REFMAC (see Murshudov, G. N., Vagin, A. A., and Dodson, E. J. (1997) Refinement of macromolecular structures by the maximum likelihood method. Acta Crystallogr. D53, 240-255) with tight non-crystallographic symmetry restraints and performed TLS refinement. At last phase of refinement, water molecules were added. The final structure of IL-36α—L249B Fab complex was refined to residual factors R/Rfree=22.0/26.7.

Correspondingly, the structure of IL-36γ—L249B Fab complex was also determined by molecular replacement in PHASER-MR using IL-36γ structure (PDB 4IZE) and mouse IgG1 Fab F124 (anti-hepatitis B surface antigen MAb, PDB ID 1F11) as search models. The MR output was further refined with PHENIX/REFMAC and BUSTER (see Bricogne G., B. E., Brandl M., Flensburg C., Keller P., Paciorek W., and Roversi P, S. A., Smart O. S., Vonrhein C., Womack T.O. (2017) BUSTER version X.Y.Z.) with tight non-crystallographic symmetry restraints and basic TLS refinement. The surface exposed loops of IL-36γ and the constant region of L249B Fab in the IL-36γ—L249B Fab complex were built in the Fo-Fc electron density map gradually by cycles of iterative manual model building with program COOT and ARP/wARP function (see Morris, R. J., Perrakis, A., and Lamzin, V. S. (2003) ARP/wARP and automatic interpretation of protein electron density maps. Methods Enzymol 374, 229-244) as part of the CCP4 suite. Water molecules were added and the final structure was refined to residual factors R/Rfree=23.5/27.8.

Both the complex structures have good geometry with 4 residues (0.89%) as outliers and 97.6% residues in favored region of the Ramachandran plot. The data collection and refinement statistics are summarized in Table 21. All figures were made in PyMOL (see DeLano, W. (2002) The PyMOL Molecular Graphics System).

To understand the binding mode of humanized L249B Fab towards IL-36α and IL-36γ, the crystal structures of both IL-36α—L249B Fab and IL-36γ—L249B Fab complexes were individually determined. The structures were determined by molecular replacement method and refined to a resolution of 2.7 and 2.65 Å respectively. In both complexes, interactions between the Fab variable domain and the cytokine were clearly visible in the electron density.

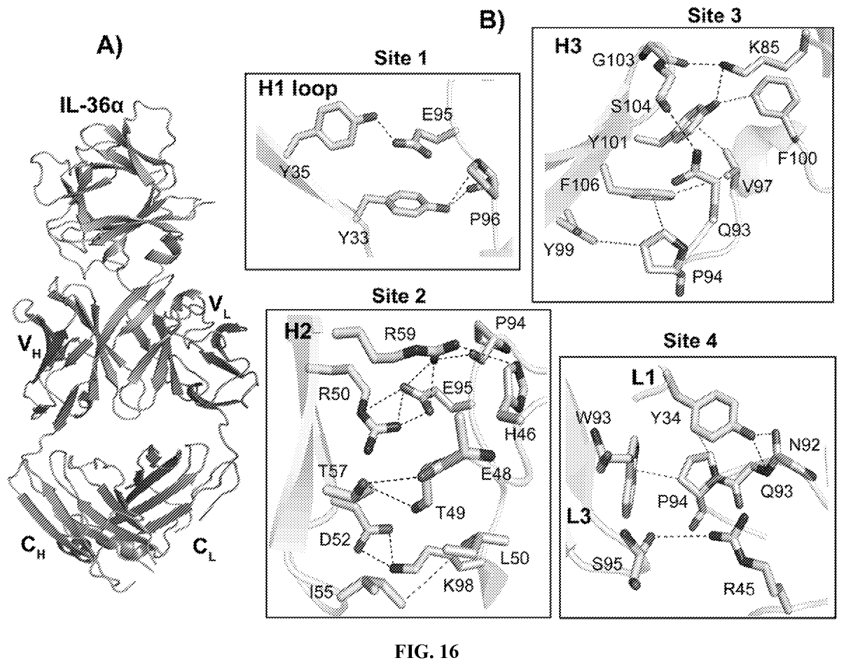

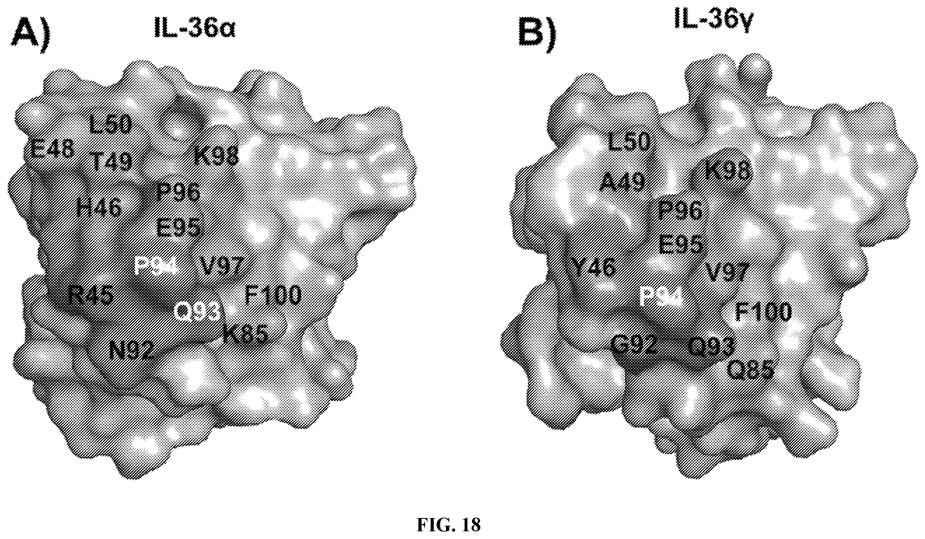

The asymmetric unit in the crystal contained two copies of IL-36α—L249B Fab complex and in each copy one IL-36α molecule binds to one Fab, leading to a 1:1 arrangement (FIG. 16, part A). In both copies, the structure of IL-36α (1-153 a.a) and the Fab variable domain (1-120 a.a of heavy chain and 1-110 a.a of light chain) were well ordered while some of the surface exposed loops of Fab constant domain and its C-terminal region (136-143; 194-197; 223-228 residues of heavy chain and 151-154; 210-215 residues of light chain) were disordered, which might be due to their greater flexibility. In the complex, the β-trefoil fold of IL-36α is conserved with 12 β-strands connected by loops. IL-36α employs residues from the loops connecting β4-β5 strands and β7-β8 strands to interact with the complementary determining region (CDR) loops of variable domain of both heavy chain (HC) and light chain (LC) of Fab. The variable region of L249B Fab binds to a total of 14 discontinuous IL-36α residues. The binding interface is predominantly formed by HC residues with a total buried surface area of 1138 Å² and interface area of 553 Å², while the LC buries a total of 353 Å² with reduced interface area of 157 Å². The HC also dominates the overall interactions and binds to 12 IL-36α residues (His 46, Glu 48, Thr 49, Leu 50, Lys 85, Gln 93, Pro 94, Glu 95, Pro 96, Val 97, Lys 98 and Phe 100), while the LC contacts only four IL-36α residues (Arg 45, Asn 92, Gln 93 and Pro 94). At the binding interface, Gln 93 and Pro 94 are the two residues of IL-36α that interact with both heavy chain and light chain of L249B Fab (FIG. 18, part A).

Detailed inspection revealed that polar, electrostatic (salt-bridges) and hydrophobic contacts are formed throughout the binding interface, and play major role in the complex formation. Throughout the interface both IL-36α and L249B Fab residues mostly employ their side chain atoms to interact with each other. In addition, few hydrogen bonding contacts between the main chain carbonyl and amide groups of cytokine and the antibody were also observed. The interface can be divided into four major binding sites, out of which, three sites were formed by HC CDR loops H1, H2 and H3 and the fourth binding site mediated by LC CDR loops L1 and L3. (FIG. 16, part B). L2 is the only CDR loop of L249B Fab that does not interact with any residue of IL-36α.

All three HC CDRs take part in the complex formation between IL-36α and L249B Fab, forming an intricate H-bond network of 11 H-bonds and 6 salt bridges. Site 1 contains a small patch of interaction interface wherein, the H1 loop residues Tyr 33 and Tyr 35 recruit their hydroxyl (OH) group to facilitate hydrogen-bonding contact with respective main chain carbonyl group of Pro 96 and side chain carboxyl group of Glu 95 of IL-36α. Site 2 is the major interaction area holding key salt-bridge contacts between cytokine and the Fab molecule. At this region, Arg 50 and Arg 59 of H2 loop makes salt-bridge with Glu 95 of IL-36α while the Asp 52 forms salt-bridge contact with Lys 98 (IL-36a). In addition, Arg 59 also mediates hydrogen-bonding contact with main chain carbonyl group of Pro 94 and side chain imidazole ring of His 46 of IL-36α. Furthermore, at this site, Thr 57 is in hydrogen-bonding distance with Thr 49 and Glu 48 of IL-36α while the Ile 55 relates with Leu 50 (IL-36α) via hydrophobic contact. Site 3 also encompasses a wide-range of interface in which along with polar interactions, hydrophobic contacts between H3 loop residues and IL-36α were identified. At this site, the main chain carbonyl groups of Gly 103 and Ser 104 of L249B Fab makes hydrogen bonding contact with side chain atoms of Lys 85 and Gln 93 of IL-36α respectively. The Lys 85 also makes one more polar contact with hydroxyl group of Tyr 101 (H3 loop). In addition to these polar contacts, we have seen a stretch of hydrophobic interactions among H3 loop residues Tyr 99, Tyr 101 and Phe 106 with IL-36α residues Pro 94, Val 97 and Phe 100.

Contrary to the extended interface observed between HC and IL-36α, the interaction interface mediated by LC residues at site 4 is relatively smaller with only three polar contacts that were detected at this region. First, the Tyr 34 of L1 loop employs its hydroxyl group to make a hydrogen bond with Asn 92 and Gln 93 of IL-36a. Further, the main chain carbonyl group of Ser 95 of L3 loop is in hydrogen bonding distance with the amide nitrogen of Arg 45 of IL-36α. At this region, the IL-36α residue Pro 94, mediates hydrophobic contact with both Tyr 34 (L1) and Trp 93 of (L3).

In summary, IL-36α residues Glu 95 and Lys 98 are critical to favor key electrostatic interactions along with Pro 94 and Val 97 that promote various hydrophobic contacts; altogether strongly stabilizes the IL-36α—L249B Fab complex formation.

The asymmetric unit of the crystal contains one copy of IL-36γ—L249B Fab complex. In the final structure, with the exception of some flexible loops and C-terminal end (138-141 a.a; 224-228 a.a of HC and 210-215 a.a of LC) most of the L249B Fab structure was well ordered. However, we have not observed obvious electron density corresponding to some surface exposed loops of IL-36γ (20-29 a.a; 136-144 a.a) far from the antibody-binding site, which may be due to crystal packing. The global structure of the IL-36γ in complex with L249B Fab is analogous to unbound IL-36γ (PDB 4IZE) with minimal structural adaptations in its surface exposed loops (root mean square deviation value (RMSD) of 0.5 Å between 115 Cα atoms from both molecules). This recommends that the antibody binding did not induce any major conformational change in the over all structure of the cytokine. Likewise, the structural superposition of both IL-36α and IL-36γ complexed with L249B Fab also results in an overall RMSD value of less than 0.7 Å between 110 CA atoms with identical topological architecture and similar antibody binding region.

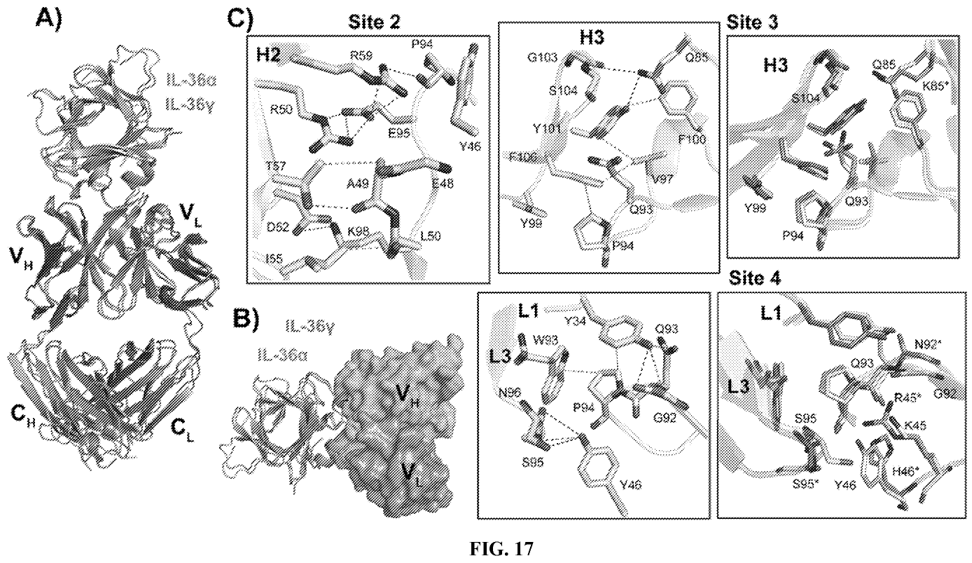

The structure of the IL-36α—L249B Fab complex was compared with IL-36γ—L249B Fab complex by aligning the structurally similar cytokine molecule. The structural superposition revealed that both complexes align with an RMSD value of ˜1.2 Å between 419 Cα atoms in which both cytokines and the variable region of L249B Fab superpose perfectly. However, in both complexes, the constant region deviates to some degree in terms of its relative orientation with respect to variable domain (FIG. 17A). Similar to IL-36α, the IL-36γ also binds at the deep crevice formed by the CDR loops from both HC and LC of Fab (FIG. 17B). The interaction interface area between the HC (10 residues) and IL-36γ (9 residues) is 549 Å² while the interface between IL-36γ and LC involves 4 residues of each with total interaction area of 220 Å², signifying that HC has higher contribution in stabilizing the IL-36γ—L249B Fab complex formation (FIG. 18, part B).

Our structural alignment revealed that IL-36α and IL-36γ possess analogous Fab binding interface in terms of recognizing similar epitope with majority of Fab interacting residues conserved between them. Correspondingly, L249B Fab also utilizes similar residues to bind to these two cytokines (FIG. 17, part C). Most of the interactions that were seen at site 1 and site 2 regions of IL-36α—L249B Fab complex are well maintained in IL-36γ—L249B Fab complex except two differences at site 2. First, the hydrogen-bonding contact between Arg 59 and His 46 is missing in the IL-36γ—L249B Fab complex, as IL-36γ possesses Tyr 46 and the hydroxyl group of Tyr 46 is too far to make contact with Arg 59. Second, in IL-36γ, due to slight structural adaptation in the loop spanning residues Glu 48 and Ala 49, the hydrogen bond between Thr 57 (H2 loop) and Glu 48 is missing. Instead, at this region Ala 49 makes additional hydrophobic contact with Thr 57 (H2). Likewise, at site 3 while the key interactions are conserved, the side chain of Gln 93 (IL-36γ) adopts different rotamer to prevent steric clash with surrounding residues, due to which IL-36γ lacks interaction with Ser 104 of H3 loop. In addition, the conformational change in the H3 loop of IL-36γ—L249B Fab complex prevents a hydrophobic interaction between Tyr 99 (H3) and Pro 94 (IL-36γ).

Overall, the HC binding interface is comparable in both IL-36α and IL-36γ, however, considerable variations exist in both cytokines with respect to LC binding in the context of specific amino acids used to form the individual contacts. The LC of L249B Fab retains unique interactions with IL-36γ residues Gln 93 and Tyr 46 that were not observed in IL-36α—L249B Fab complex and thereby makes more hydrogen bonding contacts with IL-36γ in comparison to IL-36α. First, the unique orientation of side chain of Gln 93 of IL-36γ brings it closer to hydroxyl group of Tyr 34 of L1 loop to make hydrogen-bonding contact. Second, the hydroxyl group of Tyr 46 of IL-36γ (His 46 in IL-36α) mark hydrogen bonds with Ser 95 and Asn 96 of L3 loop.

Here, the hydrogen bond seen between Arg 45 of IL-36α and Ser 95 of L3 is missing in IL-36γ—L249B Fab complex as IL-36γ possesses Lys 45 and the lysyl group is too short to make any contact with L249B Fab residues. While the polar interface at LC region is different in IL-36α—L249B Fab and IL-36γ—L249B Fab complex, the hydrophobic network between LC residues and the cytokine is conserved in both complexes.

In summary, the contribution of LC region is significantly higher in IL-36γ—L249B Fab complex compared to IL-36α—L249B Fab complex. Interactions between IL-36α or IL-36γ and heavy chain/light chain regions of L249B Fab are summarized in Table 22 and Table 23.

TABLE 21
Data collection and refinement statistics for IL-36α -
L249B Fab complex and IL-36γ - L249B Fab complex.

| | IL-36α - L249B Fab | | IL-36γ - L249B Fab | |
|---|---|---|---|---|
| Data collection statistics | complex | | complex | |
| PDB ID | | | | |
| Space group | H3₂ | | P 6₄ 2 2 | |
| Cell dimension | | | | |
| a, b, c, (Å) | 148.0, 148.0, 410.7 | | 112.9, 112.9, 199.7 | |
| α, β, γ (°) | 90.00, 90.00, 120.00 | | 90.00, 90.00, 120.00 | |
| Resolution range (Å) | 80-2.7 | (2.79-2.7) | 97-2.65 | (2.91-2.65) |
| [outer shell] | | | | |
| No. of unique reflections | 47934 | (4661) | 15346 | (767) |
| Rₘₑₐₛ (%) | 11.3 | (129.5) | 12.6 | (260.1) |
| Rₚᵢₘ (%) | 5.3 | (59.3) | 2.3 | (46.9) |
| Multiplicity | 10.5 | (11.0) | 34.2 | (30.7) |
| Average I/σ | 10.1 | (2.0) | 20.9 | (2.1) |
| Completeness (%) | 99.9 | (100) | 92.8 | (79.0) |

| | | |
|---|---|---|
| Refinement statistics | | |
| No. atoms | | |
| Protein | 8327 | 4055 |
| Sulphate/glycerol | 70 | 0 |
| Water | 221 | 155 |
| Ramachandran plot (%) | | |
| Favored | 94.7 | 93.2 |
| Allowed | 4.3 | 6.1 |
| Outliers | 0.9 | 0.7 |
| R.m.s. deviations | | |
| Bonds (Å) | 0.013 | 0.013 |
| Angles (°) | 1.41 | 1.32 |
| B-factors (Å²) | | |
| Protein | 57.9 | 54.7 |
| Sulphate/glycerol | 58.4 | 0 |
| Water | 68.8 | 54.9 |
| R factor (%) | 22.0 | 23.5 |
| R_{free} (%) | 26.7 | 27.8 |

TABLE 22
Interactions between IL-36α and heavy chain/light
chain regions of L249B Fab (the surface contacts between
IL-36α and L249B Fab were calculated using PISA server)

| | | |
|---|---|---|
| Antigen residues | Antibody residues | Hydrogen bonding |
| (IL-36α) | (L249B Fab) | distance (Å) |

Polar interactions of IL-36α with heavy chain region of L249B Fab
H1 loop

| | | |
|---|---|---|
| Pro 96 (O) | Tyr 33 (OH) | 2.7 |
| Pro 96 (N) | Tyr 33 (OH) | 3.6 |
| Glu 95 (OE2) | Tyr 35 (OH) | 2.4 |

H2 loop

| | | |
|---|---|---|
| Lys 98 (NZ) | Asp 52 (OD1) | 2.9 (S.B) |
| Lys 98 (NZ) | Asp 52 (OD2) | 2.9 (S.B) |
| Glu 95 (OE1) | Arg 50 (NH1) | 2.6 (S.B) |
| Glu 95 (OE2) | Arg 50 (NH1) | 3.1 (S.B) |
| Glu 95 (OE2) | Arg 50 (NE) | 3.6 |
| Pro 94 (O) | Arg 59 (NH1) | 2.9 |
| Glu 95 (OE1) | Arg 59 (NH1) | 2.9 (S.B) |
| Glu 95 (OE2) | Arg 59 (NH1) | 3.2 (S.B) |
| His 46 (ND1) | Arg 59 (NH2) | 3.7 |
| Glu 48 (O) | Thr 57 (OG1) | 3.7 |
| Thr 49 (O) | Thr 57 (OG1) | 3.9 |

H3 loop

| | | |
|---|---|---|
| Gln 93 (NE2) | Ser 104 (O) | 3.2 |
| Lys 85 (NZ) | Gly 103 (O) | 2.5 |
| Lys 85 (NZ) | Tyr 101 (OH) | 2.8 |

Polar interactions of IL-36α with light chain region of L249B Fab

| | | |
|---|---|---|
| Gln 93 (N) | Tyr 34 (OH) | 3.5 |
| Asn 92 (O) | Tyr 34 (OH) | 2.6 |
| Arg 45 (NH2) | Ser 95 (O) | 3.8 |

Van der Waals' contacts between IL-36α and L249B Fab

| | | |
|---|---|---|
| Pro 94 | Tyr 34 (LC) | 3.9 |
| Pro 94 | Trp 93 (LC) | 3.4 |
| Val 97 | Tyr 101 (HC) | 3.5 |
| Val 97 | Phe 106 (HC) | 3.8 |
| Phe 100 | Tyr 101 (HC) | 4.5 |
| Pro 94 | Phe 106 (HC) | 4.3 |
| Pro 94 | Tyr 99 (HC) | 4.2 |
| Leu 50 | He 55 (HC) | 4.7 |

*S.B indicates salt bridge contact.

TABLE 23
Interactions between IL-36γ and heavy chain/light
chain regions of L249B Fab (the surface contacts between
IL-36γ and L249B Fab were calculated using PISA server)

| | | |
|---|---|---|
| Antigen residues | Antibody residues | Hydrogen bonding |
| (IL-36γ) | (L249B Fab) | distance (Å) |

Polar interactions of IL-36γ with heavy chain region of L249B Fab
H1 loop

| | | |
|---|---|---|
| Pro 96 (O) | Tyr 33 (OH) | 2.7 |
| Glu 95 (OE1) | Tyr 35 (OH) | 2.7 |

H2 loop

| | | |
|---|---|---|
| Lys 98 (NZ) | Asp 52 (OD1) | 3.8 (S.B) |
| Lys 98 (NZ) | Asp 52 (OD2) | 3.3 (S.B) |
| Glu 95 (OE1) | Arg 50 (NH1) | 3.8 (S.B) |
| Glu 95 (OE2) | Arg 50 (NH1) | 2.8 (S.B) |
| Glu 95 (OE1) | Arg 50 (NE) | 2.8 |
| Pro 94 (O) | Arg 59 (NH1) | 3.1 |
| Glu 95 (OE2) | Arg 59 (NH2) | 3.2 (S.B) |
| Glu 95 (OE2) | Arg 59 (NH1) | 3.4 (S.B) |
| Ala 49 (O) | Thr 57 (OG1) | 3.1 |

H3 loop

| | | |
|---|---|---|
| Gln 85 (NE2) | Gly 103 (O) | 3.7 |
| Gln 85 (NE2) | Tyr 101 (OH) | 3.0 |

Polar interactions of IL-36γ with light chain region of L249B Fab

| | | |
|---|---|---|
| Gln 93 (N) | Tyr 34 (OH) | 3.6 |
| Gly 92 (O) | Tyr 34 (OH) | 3.0 |
| Gln 93 (OE1) | Tyr 34 (OH) | 3.6 |
| Tyr 46 (OH) | Ser 95 (OG) | 3.3 |
| Tyr 46 (OH) | Asn 96 (N) | 3.4 |
| Tyr 46 (OH) | Ser 95 (O) | 3.7 |

Van der Waals' contacts between IL-36γ and L249B Fab

| | | |
|---|---|---|
| Pro 94 | Tyr 34 (LC) | 3.8 |
| Pro 94 | Trp 93 (LC) | 3.9 |
| Val 97 | Tyr 101 (HC) | 3.6 |
| Phe 100 | Tyr 101 (HC) | 4.1 |
| Val 97 | Phe 106 (HC) | 3.5 |
| Pro 94 | Phe 106 (HC) | 4.7 |
| Leu 50 | Ile 55 (HC) | 4.5 |
| Ala 49 | Thr 57 (HC) | 4.3 |

### 6.9 Example 9—Crystallization of IL-36α—144D464A Fab Complex and IL-36γ—144D464A Fab Complex

Purified chimeric mouse/human 144D464A Fab (D464A) and IL-36α and IL-36γ proteins were mixed separately in a 1:1 stoichiometric ratio, concentrated to 3.2 mg/ml and then subjected to crystallization. Initial crystallization trials for both complexes were performed in a 96-well format using a nano-liter dispensing liquid handling robot (Phenix, Art Robbins Ltd.). Over 600 different commercially available crystallization screens (JCSG core+, JCSG core 1-4 screens, Sigma) were tested by sitting drop vapor diffusion method at both 4° C. and 22° C. Optimization of all crystallization conditions was performed manually by both hanging drop and sitting drop methods. Crystals of IL-36α—464 Fab complex grew over 7 days in various conditions having PEG 6000 as a common precipitant. All these crystallization conditions were further improved by equilibrating 1.2 μl of protein (3.6 mg/ml IL-36α—D464A Fab complex in 50 mM HEPES, pH 7.0 and 150 mM NaCl) and 0.8 μl of reservoir solution at both 4° C. and 22° C. Among all conditions, the crystals generated at 4° C. by sitting drop method in a well solution consisting of 20% WN PEG 6000, 0.1M HEPES and 1.0M Lithium Chloride generated high quality-diffraction. On the other hand, the crystals of IL-36γ—D464A Fab complex used for X-ray diffraction experiment were grown by hanging drop vapor diffusion method at 4° C. by equilibrating a mixture containing 1.5 μ1 of protein complex (3.2 mg/ml IL-36γ—D464A Fab complex in in 50 mM HEPES, pH 7.0 and 150 mM NaCl) and 1 μl of reservoir solution containing 20% (w/v) PEG 3000, 0.1M Imidazole pH 8.0 and 0.2 M Zinc acetate, against 1 ml of reservoir solution. Prior to diffraction, all crystals were cryoprotected by immersing in mother liquor containing 20% glycerol and were flash-cooled in liquid nitrogen for subsequent data collection.

Data collection and refinement were performed similarly as in Example 8 above. More specifically, native X-ray diffraction data for all crystals were collected remotely at Stanford Synchrotron Radiation Light Source beamline 9-2 using a PILATUS 6M PAD detector at a wavelength of 0.97 Å and at 100 K temperature. The diffraction data images for both complexes were collected with 0.15-degree oscillation and 1 sec exposure time. The data images were indexed, integrated and scaled in HKL 2000 package to an overall resolution of 2.3 Å for both IL-36α—D464A Fab complex and IL-36γ—D464A Fab complex. Both the complexes belong to the space group P2₁, with unit cell dimensions: α=78.02 Å, b=68.02 Å, c=111.04 Å, α=90°, β=92.49°, γ=90° and a=79.52 Å, b=70.85 Å, c=111.73 Å, α=90°, β=99.12°, γ=90° respectively. The position of IL-36α in the asymmetric unit was determined by molecular replacement method PHASER-MR using the previously reported IL-36γ structure (PDB 4IZE) as a search model. Similarly, the position of D464A Fab was also identified by molecular replacement method using the L and H chain of the mouse IgG1 Fab F124 (anti-hepatitis B surface antigen MAb, PDB ID 1F11) separately, as a starting search model. Starting with initial phases obtained by MR, first the D464A Fab model was built gradually by cycles of iterative model building and subsequently some regions of IL-36α was manually built into the Fo-Fc electron density map using COOT function as part of the CCP4 suite. The models were further refined using PHENIX/REFMAC with tight non-crystallographic symmetry restraints. At last phase of refinement, water molecules were added. The final structure of IL-36α—D464A Fab complex was refined to residual factors R/Rfree=20.7/25.1. Correspondingly, the structure of IL-36γ—D464A Fab complex was also determined by molecular replacement using the phase information obtained from IL-36α—D464A Fab complex and the structure was refined to 2.3 Å with residual factors R/Rfree=20.3/25.2. Both the complex structures have good geometry with 4 residues (0.89%) as outliers and 97.6% residues in favored region of the Ramachandran plot. The data collection and refinement statistics are summarized in Table 24. All figures were made in PyMOL.

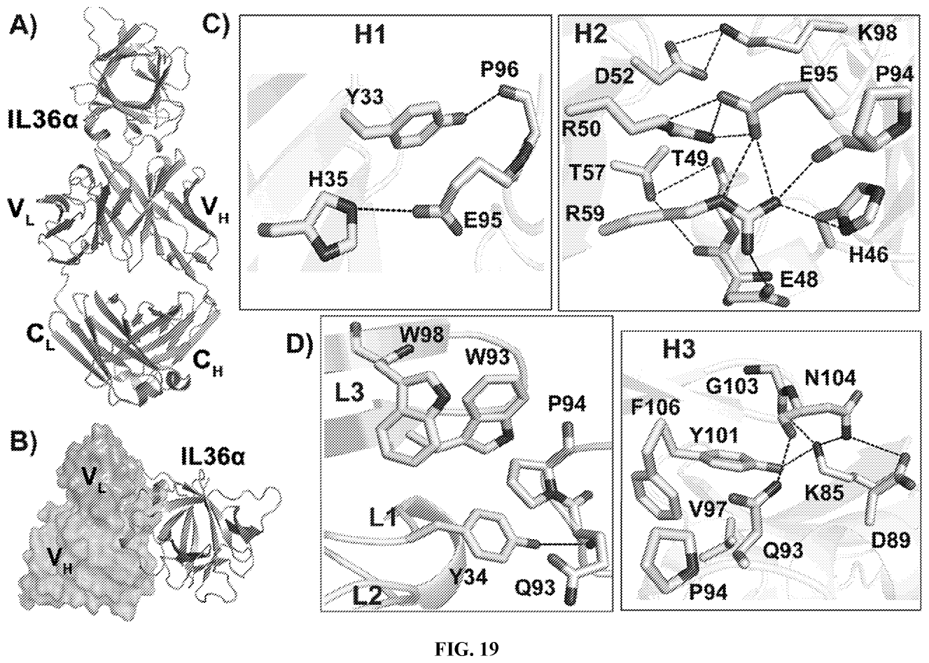

The asymmetric unit of the crystal contains two copies of the IL-36α—D464A Fab complex. Each molecule of cytokine binds to one molecule of D464A Fab, leading to a 1:1 arrangement as the minimal biological unit. (FIG. 19, part A). In the final structure, with the exception of few flexible loops, most of the IL-36α and D464A Fab structures were well ordered. The final model consists of D464A Fab light chain residues 1-211 and heavy chain residues 1-222 in each molecule. Likewise, proper electron density was observed for IL-36α residues 2-151 and the cytokine exhibits typical β-trefoil fold composed of 12 β-strands connected by loops which is similar to that found in all other IL-1 family Interleukins. In the complex structure, IL-36α employs residues from its loop regions to bind at the crevice formed by the complementary determining region (CDR) loops from both heavy chain and light chain (FIG. 19, part B). In the complex, IL-36α extensively interacts with heavy chain region of D464A Fab with an interface area of ˜600 Å² while it shares considerably reduced interface area of ˜155 Å² with the light chain region (FIG. 19, part C and part D). Hence, the heavy chain has higher contribution in contacting with IL-36α in comparison to light chain. At the interface, a total of 11 residues from cytokine and 10 residues from heavy chain and 3 residues from light chain participate in stabilizing the IL-36α—D464A Fab complex. Detailed inspection revealed that polar and hydrophobic contacts are distributed in the interaction interface. In addition, we have also observed key electrostatic interactions (salt-bridges) between heavy chain region of Fab and IL-36α that play a major role in the complex formation. Majority of polar residues from both the cytokine and the Fab recruit their side chain atoms to participate in the hydrogen bonding contacts while a slight contribution from main chain carbonyl and amide groups is also observed. The binding between IL-36α and three CDR loops of the heavy chain (H1, H2 and H3) region of Fab is mostly facilitated by polar and charged interactions. Conversely, the interaction between IL-36α and light chain majorly occurs via hydrophobic contacts and only a single hydrogen bonding interaction was found with the light chain CDR1 but not with the L2 and L3 of D464A Fab. We divided the binding interface into four major binding sites. Site 1 corresponds to the interaction of the IL-36α with H1 loop; site 2, interaction of the IL-36α with H2 loop; site 3, interaction of the IL-36α with H3 loop and site 4, interaction of the IL-36α with light chain region of D464A Fab. In total, IL-36α makes 20 polar contacts with the antibody including nine salt bridges (Table 25).

Site1 contains a relatively small contact area in which, Glu 95 of IL-36α makes hydrogen-bonding contact with His 35 of H1 loop while the main chain carbonyl group of Pro 96 contacts with hydroxyl group of Tyr 33 of H1 loop (FIG. 19, part C, left panel). Site 2 consists of an elongated interface area and this region stabilizes the IL-36α—D464A Fab complex by forming salt-bridge contacts between the cytokine and the Fab molecule. Firstly, Arg 50 and Arg 59 of H2 loop forms salt-bridge with Glu 95 of IL-36α, while the latter also makes one more salt-bridge contact with Glu 48 of IL-36a. Second, Asp 52 of H2 loop interacts with Lys 98 of IL-36α via salt-bridge formation. In addition, we have also observed hydrogen-bonding interactions between Arg 59 and Thr 57 of H2 loop and Pro 94, His 46, Glu 48 and Thr 49 of IL-36α (FIG. 19, part C, right panel). Further, in this region, Leu 50 of IL-36α makes hydrophobic contact with Ile 55 of H2 loop. At site 3, Asn 104 of H3 loop is in hydrogen bonding distance with the IL-36α residues Gln 93, Asp 89 and Lys 85. Similarly, Lys 85 also interacts with Gly 103 and Tyr 101 of heavy chain CDR3 loop. At site 3, along with polar contacts, there also exist Van der Waals' interactions between aromatic ring of Tyr 101 and Phe 106 with Val 97 and Pro 94 of IL-36α (FIG. 19, part C, right panel). Contrary to all the three binding sites of heavy chain region, the interaction interface at site 4 is smaller and is mostly mediated by hydrophobic contacts. At this region, the amide group nitrogen atom of Gln 93 of IL-36α makes hydrogen-bonding contact with hydroxyl group of Tyr 34 of light chain CDR1 loop. Additionally, Pro 94 of IL-36α contacts with aromatic side chains of Tyr 34 and Trp 93 of L1 and L3 loops of D464A Fab by hydrophobic interactions (FIG. 19, part D, left panel). L2 is the only CDR that does not interact with any of the IL-36α residues. In summary, the binding interface of IL-36α—D464A Fab complex revealed that Glu 95 of IL-36α is the key residue that makes majority of salt-bridge contacts with the D464A Fab. Further, two more residues of IL-36α, Pro 94 and Gln 93 interact with both heavy chain and light chain regions of Fab where in Pro 94 is responsible for making Van der Waals' interactions with both heavy chain and light chain residues. Similarly, the main chain carbonyl group of Gln 93 is interacting with light chain where as its side chain is responsible for hydrogen bonding with heavy chain residue Tyr 101.

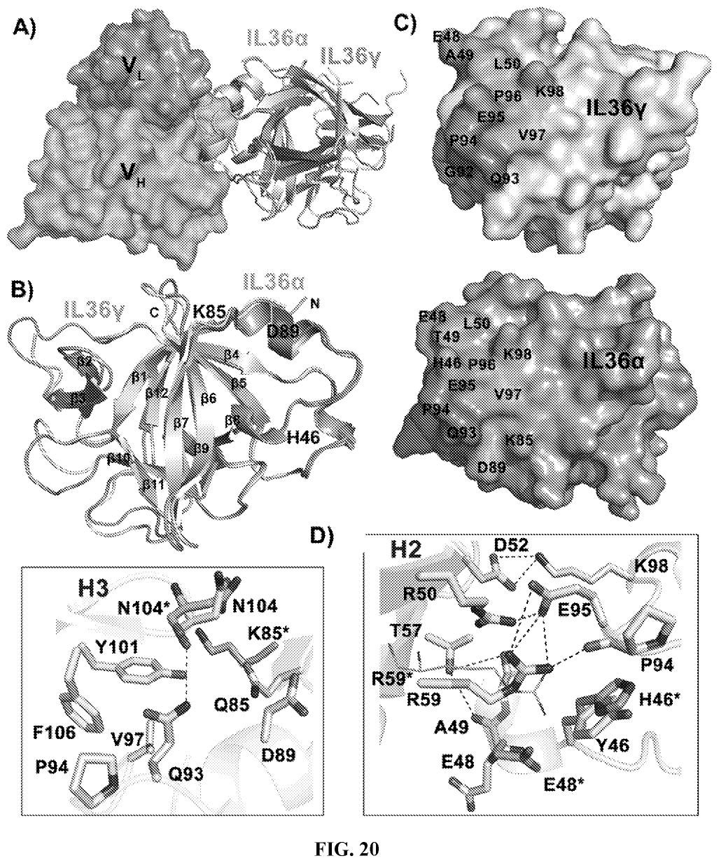

The crystals of IL-36γ—D464A Fab complex also contain 2 individual complexes in the asymmetric unit with both the copies superposing perfectly with each other. In the final model, the heavy chain residues 1-225, light chain residues 2-212 and IL-36γ residues 3-151 are ordered in both the complexes. The crystal structure of IL-36γ in complex with Fab is almost identical to the previously reported unbound IL-36γ (PDB 4IZE) and the binding of D464A Fab did not induce any conformational change in the cytokine architecture. Similar to IL-36α—D464A Fab complex structure, IL-36γ also binds at the crevice formed by CDR loops of both heavy chain and light chain region of D464A Fab (FIG. 20, part A). In the complex, IL-36γ majorly interacts with heavy chain loops of D464A Fab via polar and electrostatic interactions and to little extent with light chain CDRs L1 and L3 by polar and hydrophobic contacts (Table 26). The overall structure of IL-36γ—D464A Fab complex appears similar to the crystal structure of IL-36α—D464A Fab complex with an overall root mean square deviation (RA/ND) value of less than 1.2 A between 481 CA atoms from both complexes. Both IL-36α and IL-36γ share around 55% sequence similarity with identical topological architecture. Structural superposition of IL-36α and IL-36γ, both in complex with D464A Fab reveals that the antibody binding region is similar between them. However, the loops connecting strands β3-β4, β5-β6, β6-β7 and β10-β11 exhibit conformational changes between both cytokines (FIG. 20, part B). Whether these structural adaptations between both cytokines might exist in general or induced by the binding of antibody is still inconclusive.

Comparison of the binding interface between both complexes disclosed that most of the D464A Fab interacting residues are conserved between IL-36α and IL-36γ. The D464A Fab binding foot print on IL-36α and IL-36γ revealed that the hydrogen bonding contacts mediated by His 46, Lys 85 and Asp 89 of IL-36α are absent in IL-36γ as the later cytokine lacks the interacting residues at this region (FIG. 20, part C). As a result, while the H1 loop interactions of D464A Fab are conserved between IL-36α and IL-36γ, a few polar contacts were missing between H2 loop and IL-36γ that were otherwise present in IL-36α—D464A Fab complex. In place of His 46 of IL-36α, IL-36γ has a tyrosine residue (Tyr 46) and because of the bulkier nature of the aromatic ring of Tyr 46, the following residue Glu 48 obtains a completely different orientation compared to that of IL-36α. This orientation relocates the side chain of Glu 48 far away from Arg 59 of H2 loop hence, the salt-bridge contact present between Glu 48 and Arg 59 in IL-36α—D464A Fab complex is missing in the IL-36γ—D464A Fab complex (FIG. 20, part D, right panel). Likewise, though most of the H3 loop interactions are conserved between both cytokines, the side chain of Gln 85 of IL-36γ (Lys 85 in IL-36α) is shorter compared to the longer lysyl group present in Lys 85. Due to this replacement, IL-36γ lacks polar contacts with Asn 104, Gly 103 and Tyr 101 of H3 loop. Additionally, at this region, slight reorientation of Asn 104 side chain further prevents its interaction with Asp 89 of IL-36γ (FIG. 20, part D, left panel). Similar to heavy chain binding, the polar and hydrophobic contacts with the light chain binding region of D464A Fab are also preserved between IL-36α and IL-36γ. However, in the IL-36γ—D464A Fab complex, the hydroxyl group of Tyr 34 of light chain CDR1 loop makes hydrogen bonding contact with Gly 92 in addition to maintaining the conserved interaction with Gln 93.

The crystallography data reveals conserved contact residues shared by IL-36α and IL-36γ for binding by 144L249B and 144D464A. It is noted that several of these residues are conserved between IL-36α (SEQ ID NO: 5 or SEQ ID NO: 7) and IL-36γ (SEQ ID NO: 10) but not with IL-36β (SEQ ID NO: 9), suggesting their role in the dual specificity of the 144L249B and 144D464A monoclonal antibodies. The conserved contact residues in IL-36α and IL-36γ include Leu 50, Gln 93, Pro 94, Glu 95, Pro 96, Val 97, and Lys 98.

TABLE 24
Data collection and refinement statistics for IL-36α -
D464A Fab complex and IL-36γ - D464A Fab complex

| | IL-36α - D464A Fab | | IL-36γ - D464A Fab | |
|---|---|---|---|---|
| Data collection statistics | complex | | complex | |
| PDB ID | | | | |
| Space group | P2₁ | | P2₁ | |
| Cell dimension | | | | |
| a, b, c, (Å) | 78.02, 68.02, 111.04 | | 79.52, 70.85, 111.73 | |
| α, β, γ (°) | 90.00 92.49 90.00 | | 90.00 99.12 90.00 | |
| Resolution range (Å) | 70-2.3 | (2.34-2.3) | 50-2.3 | (2.34-2.3) |
| [outer shell] | | | | |
| No. of unique reflections | 50154 | | 53630 | |
| Rₘₑₐₛ (%) | 7.7 | (25.4) | 13.6 | (74.1) |
| Rₚᵢₘ (%) | 3.6 | (13.2) | 6.6 | (37.5) |
| Multiplicity | 4.3 | (3.4) | 4.0 | (3.7) |
| Average I/σI | 22.8 | (3.8) | 14.8 | (1.85) |
| Completeness (%) | 97.0 | (86.8) | 98.5 | (98.5) |

| | | |
|---|---|---|
| Refinement statistics | | |
| No. atoms | | |
| Protein | 8653 | 8775 |
| ligand | 25 | 40 |
| Water | 401 | 372 |
| Ramachandran plot (%) | | |
| Favored | 95.7 | 96.2 |
| Allowed | 3.4 | 3.1 |
| Outliers | 0.8 | 0.7 |
| R.m.s. deviations | | |
| Bonds (Å) | 0.013 | 0.010 |
| Angles (°) | 1.61 | 1.35 |
| B-factors (Å²) | | |
| Protein | 41.3 | 32.7 |
| ligand | 36.1 | 27.1 |
| Water | 37.2 | 34.5 |
| R factor (%) | 20.7 | 20.3 |
| R_{free} (%) | 25.1 | 25.2 |

TABLE 25
Interactions between IL-36α and heavy chain/light
chain regions of D464A Fab (the surface contacts between
IL-36α and D464A Fab were calculated using PISA server)

| | | |
|---|---|---|
| Antigen residues | Antibody residues | Hydrogen bonding |
| (IL-36α) | (D464A Fab) | distance (Å) |

Polar interactions of IL-36α with heavy chain region of D464A Fab
H1 loop

| | | |
|---|---|---|
| Pro 96 (O) | Tyr 33 (OH) | 2.7 |
| Glu 95 (OE2) | His 35 (NE2) | 3.6 (S.B) |

H2 loop

| | | |
|---|---|---|
| Lys 98 (NZ) | Asp 52 (OD1) | 2.9 (S.B) |
| Lys 98 (NZ) | Asp 52 (OD2) | 2.9 (S.B) |
| Glu 95 (OE1) | Arg 50 (NH1) | 2.7 (S.B) |
| Glu 95 (OE2) | Arg 50 (NH1) | 3.3 (S.B) |
| Glu 95 (OE2) | Arg 50 (NE) | 3.6 (S.B) |
| Pro 94 (O) | Arg 59 (NH2) | 3.1 |
| Glu 95 (OE1) | Arg 59 (NH2) | 3.0 (S.B) |
| Glu 95 (OE1) | Arg 59 (NE) | 3.2 (S.B) |
| His 46 (ND1) | Arg 59 (NH2) | 3.3 |
| Glu 48 (OE2) | Arg 59 (NH1) | 3.8 (S.B) |
| Glu 48 (O) | Thr 57 (OG1) | 3.6 |
| Thr 49 (O) | Thr 57 (OG1) | 3.7 |

H3 loop

| | | |
|---|---|---|
| Gln 93 (NE2) | Asn 104 (O) | 2.8 |
| Asp 89 (OD2) | Asn 104 (ND2) | 3.8 |
| Lys 85 (NZ) | Asn 104 (ND2) | 3.1 |
| Lys 85 (NZ) | Gly 103 (O) | 2.9 |
| Lys 85 (NZ) | Tyr 101 (OH) | 3.1 |

Polar interactions of IL-36α with light chain region of D464A Fab

| | | |
|---|---|---|
| Gln 93 (N) | Tyr 34 (OH) | 3.2 |

Van der Waals' contacts between IL-36α and D464A Fab

| | | |
|---|---|---|
| Pro 94 | Tyr 34 (LC) | 4.1 |
| Pro 94 | Trp 93 (LC) | 3.9 |
| Pro 94 | Trp 98 (LC) | 3.9 |
| Val 97 | Tyr 101 (HC) | 4.0 |
| Val 97 | Phe 106 (HC) | 4.0 |
| Pro 94 | Phe 106 (HC) | 3.9 |
| Leu 50 | Ile 55 (HC) | 3.6 |

*S.B indicates salt bridge contact.

TABLE 26
Interactions between IL-36γ and heavy chain/light
chain regions of D464A Fab (the surface contacts between
IL-36γ and D464A Fab were calculated using PISA server)

| | | |
|---|---|---|
| Antigen residues | Antibody residues | Hydrogen bonding |
| (IL-36γ) | (D464A Fab) | distance (Å) |

Polar interactions of IL-36γ with heavy chain region of D464A Fab
H1 loop

| | | |
|---|---|---|
| Pro 96 (O) | Tyr 33 (OH) | 2.7 |
| Glu 95 (OE2) | His 35 (NE2) | 2.8 (S.B) |

H2 loop

| | | |
|---|---|---|
| Lys 98 (NZ) | Asp 52 (OD1) | 3.0 (S.B) |
| Lys 98 (NZ) | Asp 52 (OD2) | 2.8 (S.B) |
| Glu 95 (OE1) | Arg 50 (NH1) | 2.5 (S.B) |
| Glu 95 (OE1) | Arg 50 (NE) | 3.8 (S.B) |
| Pro 94 (O) | Arg 59 (NH2) | 2.6 |
| Glu 95 (OE1) | Arg 59 (NH2) | 3.2 (S.B) |
| Glu 95 (OE2) | Arg 59 (NH1) | 3.4 (S.B) |
| Glu 48 (O) | Thr 57 (OG1) | 3.4 |
| Ala 49 (O) | Thr 57 (OG1) | 3.6 |

H3 loop

| | | |
|---|---|---|
| Gln 93 (NE2) | Asn 104 (O) | 3.5 |

Polar interactions of IL-36γ with light chain region of D464A Fab

| | | |
|---|---|---|
| Gln 93 (N) | Tyr 34 (OH) | 3.4 |
| Gly 92 (O) | Tyr 34 (OH) | 3.3 |

Van der Waals' contacts between IL-36γ and D464A Fab

| | | |
|---|---|---|
| Pro 94 | Tyr 34 (LC) | 4.2 |
| Pro 94 | Trp 93 (LC) | 3.5 |
| Pro 94 | Trp 98 (LC) | 3.7 |
| Val 97 | Tyr 101 (HC) | 3.7 |
| Val 97 | Phe 106 (HC) | 4.2 |
| Pro 94 | Phe 106 (HC) | 4.2 |
| Leu 50 | Ile 55 (HC) | 3.8 |
| Ala 49 | Thr 57 (HC) | 4.0 |

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. An antibody or antigen binding fragment thereof that binds to an IL-36, wherein the antibody or antigen binding fragment thereof binds to both IL-36α and IL-36γ, wherein the antibody is an antagonist of both IL-36α and IL-36γ, and wherein the antibody or antigen binding fragment thereof comprises:
(a) a heavy chain variable region (VH) comprising
(i) VH complementarity determining region 1 (CDR H1) comprising an amino acid sequence selected from a group consisting of SEQ ID NO: 68, SEQ ID NO: 71, SEQ ID NO: 75, and SEQ ID NO: 80;
(ii) VH complementarity determining region 2 (CDR H2) comprising an amino acid sequence selected from a group consisting of SEQ ID NO: 69, SEQ ID NO: 73, SEQ ID NO: 76, SEQ ID NO: 78, and SEQ ID NO: 81; and
(iii) VH complementarity determining region 3 (CDR H3) comprising an amino acid sequence selected from a group consisting of SEQ ID NO: 70, SEQ ID NO: 72, SEQ ID NO: 74, SEQ ID NO: 77, SEQ ID NO: 79, and SEQ ID NO: 82, and
(b) a light chain variable region (VL) comprising
(i) VL complementarity determining region 1 (CDR L1) comprising an amino acid sequence selected from a group consisting of SEQ ID NO: 83 and SEQ ID NO: 86;
(ii) VL complementarity determining region 2 (CDR L2) comprising an amino acid sequence selected from a group consisting of SEQ ID NO: 84, SEQ ID NO: 87, and SEQ ID NO: 90; and
(iii) VL complementarity determining region 3 (CDR L3) comprising an amino acid sequence selected from a group consisting of SEQ ID NO: 85, SEQ ID NO: 88, SEQ ID NO: 89, SEQ ID NO: 91, and SEQ ID NO: 92.

2. The antibody or antigen binding fragment thereof of claim 1, wherein the antibody or antigen binding fragment thereof simultaneously antagonizes both IL-36α and IL-36γ.

3. The antibody or antigen binding fragment thereof of claim 1, wherein
(i) the antibody or antigen binding fragment thereof binds to one or more amino acid residues selected from the 45th amino acid residue to the 100th amino acid residue of the amino acid sequence of IL-36α represented by SEQ ID NO: 5 or SEQ ID NO: 7 and/or the amino acid sequence of IL-36γ represented by SEQ ID NO: 10;
(ii) the antibody or antigen binding fragment thereof binds to one or more amino acid residues selected from Arg 45, His 46, Glu 48, Thr 49, Leu 50, Lys 85, Asp 89, Asn 92, Gln 93, Pro 94, Glu 95, Pro 96, Val 97, Lys 98 and Phe 100 of the amino acid sequence of IL-36a represented by SEQ ID NO: 5 or SEQ ID NO: 7 and/or one or more amino acid residues selected from Tyr 46, Glu 48, Ala 49, Leu 50, Gln 85, Gly 92, Gln 93, Pro 94, Glu 95, Pro 96, Val 97, Lys 98 and Phe 100 of the amino acid sequence of IL-36γ represented by SEQ ID NO: 10; and/or
(iii) the antibody or antigen binding fragment thereof binds to one or more amino acid residues selected from His 46, Glu 48, Thr 49, Leu 50, Lys 85, Gln 93, Pro 94, Glu 95, Pro 96, Val 97 and Lys 98 of the amino acid sequence of IL-36α represented by SEQ ID NO: 5 or SEQ ID NO: 7 and/or one or more amino acid residues selected from Ala 49, Leu 50, Gly 92, Gln 93, Pro 94, Glu 95, Pro 96, Val 97 and Lys 98 of the amino acid sequence of IL-36γ represented by SEQ ID NO: 10.

4.-8. (canceled)

9. The antibody or antigen binding fragment thereof of claim 1, wherein
(i) the antibody or antigen binding fragment does not bind to IL-36β;
(ii) the antibody or antigen binding fragment does not antagonize IL-36β;
(iii) the antibody or antigen binding fragment does not bind to IL-36Ra;
(iv) the antibody or antigen binding fragment does not antagonize IL-36Ra;
(v) when used in combination with IL-36Ra, the combination of IL-36Ra and the antibody or antigen binding fragment thereof antagonizes IL-36α, IL-36β and IL-36γ;
(vi) the antibody or antigen binding fragment thereof binds to human and cynomolgus macaque IL-36α and IL-36γ, and wherein the antibody is an antagonist of human and cynomolgus macaque IL-36α and IL-36γ;
(vii) the antibody or antigen binding fragment thereof does not bind to human or cynomolgus macaque IL-36β;
(viii) the antibody or antigen binding fragment thereof does not bind to human or cynomolgus macaque IL-36Ra;
(ix) the antibody or antigen binding fragment binds to human IL-36α with a K_{D} of less than 100 nM as determined by a surface plasmon resonance method, and wherein the antibody or antigen binding fragment thereof binds to human IL-36γ with a K_{D} of less than 100 nM as determined by a surface plasmon resonance method;
(x) the antibody or antigen binding fragment binds to human IL-36α with a K_{D} of less than 10 nM as determined by a surface plasmon resonance method, and wherein the antibody or antigen binding fragment thereof binds to human IL-36γ with a K_{D} of less than 10 nM as determined by a surface plasmon resonance method;
(xi) the antibody or antigen binding fragment binds to cynomolgus macaque IL-36α with a KD of less than 100 nM as determined by a surface plasmon resonance method, and wherein the antibody or antigen binding fragment thereof binds to cynomolgus macaque IL-36γ with a K_{D} of less than 100 nM as determined by a surface plasmon resonance method;
(xii) the antibody or antigen binding fragment binds to cynomolgus macaque IL-36α with a K_{D} of less than 10 nM as determined by a surface plasmon resonance method, and wherein the antibody or antigen binding fragment thereof binds to cynomolgus macaque IL-36γ with a K_{D} of less than 10 nM as determined by a surface plasmon resonance method;
(xiii) the antibody or antigen binding fragment thereof attenuates IL-36α mediated signaling and/or IL-36γ mediated signaling;
(xiv) the antibody or antigen binding fragment thereof attenuates the binding of IL-36α to IL-36 receptor and/or the binding of IL-36γ to IL-36 receptor;
(xv) the antibody or antigen binding fragment thereof attenuates IL-36 receptor mediated signaling;
(xvi) the antibody or antigen binding fragment thereof attenuates the production of one or more cytokines and/or chemokines selected from a group consisting of IL-8, IL-6, IL-10, TNFβ, IL-1β, CXCL1, CCL5, CCL20, CCL2, CCL3, CCL4, CXCL12, VEGF-A, IL-23, IL-36α, IL-36β, and IL-36γ, and/or (xvii) the antibody or antigen binding fragment thereof antagonizes both IL-36α and IL-36γ activity on an IL-36 receptor expressing cell optionally selected from a group consisting of keratinocytes, dermal fibroblasts, monocytes, and PBMCs.

10.-13. (canceled)

14. The antibody or antigen binding fragment thereof of claim 1, wherein the IL-36α and IL-36γ are human IL-36α and IL-36γ, or wherein the IL-36α and IL-36γ are cynomolgus macaque IL-36α and IL-36γ.

15.-27. (canceled)

28. The antibody or antigen binding fragment thereof of claim 1, wherein the antigen binding fragment is selected from a group consisting of a Fab, a Fab′, a F(ab′)₂, a Fv, a scFv, a dsFv, a diabody, a triabody, a tetrabody, and a multispecific antibody formed from antibody fragments.

29. The antibody or antigen binding fragment thereof of claim 1, wherein the antibody is a mouse antibody, a fully human antibody, a humanized antibody or antigen binding fragment thereof, recombinantly produced, and/or produced by a hybridoma.

30.-34. (canceled)

35. The antibody or antigen binding fragment thereof of claim 1, wherein the antibody or antigen binding fragment thereof comprises:
(i) a CDR H1 of SEQ ID NO: 68, a CDR H2 of SEQ ID NO: 69, a CDR H3 of SEQ ID NO: 70, a CDR L1 of SEQ ID NO: 83, a CDR L2 of SEQ ID NO: 84, and a CDR L3 of SEQ ID NO: 85;
(ii) a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88;
(iii) a CDR H1 of SEQ ID NO: 71, a CDR H2 of SEQ ID NO: 73, a CDR H3 of SEQ ID NO: 74, a CDR L1 of SEQ ID NO: 86, a CDR L2 of SEQ ID NO: 87, and a CDR L3 of SEQ ID NO: 88;
(iv) a CDR H1 of SEQ ID NO: 75, a CDR H2 of SEQ ID NO: 69, a CDR H3 of SEQ ID NO: 72, a CDR L1 of SEQ ID NO: 86, a CDR L2 of SEQ ID NO: 87, and a CDR L3 of SEQ ID NO: 88;
(v) a CDR H1 of SEQ ID NO: 75, a CDR H2 of SEQ ID NO: 76, a CDR H3 of SEQ ID NO: 77, a CDR L1 of SEQ ID NO: 83, a CDR L2 of SEQ ID NO: 87, and a CDR L3 of SEQ ID NO: 89;
(vi) a CDR H1 of SEQ ID NO: 71, a CDR H2 of SEQ ID NO: 78, a CDR H3 of SEQ ID NO: 79, a CDR L1 of SEQ ID NO: 86, a CDR L2 of SEQ ID NO: 90, and a CDR L3 of SEQ ID NO: 91;
(vii) a CDR H1 of SEQ ID NO: 80, a CDR H2 of SEQ ID NO: 81, a CDR H3 of SEQ ID NO: 82, a CDR L1 of SEQ ID NO: 86, a CDR L2 of SEQ ID NO: 87, and a CDR L3 of SEQ ID NO: 92: or
(viii) a CDR H1 of SEQ ID NO: 71, a CDR H2 of SEQ ID NO: 69, a CDR H3 of SEQ ID NO: 72, a CDR L1 of SEQ ID NO: 86, a CDR L2 of SEQ ID NO: 84, and a CDR L3 of SEQ ID NO: 88.

36.-54. (canceled)

55. The antibody or antigen binding fragment thereof of claim 1, wherein the antibody or antigen binding fragment thereof comprises:
(i) a VH region comprising an amino acid sequence of SEQ ID NO: 23, and a VL region comprising an amino acid sequence of SEQ ID NO: 51;
(ii) a VH region comprising an amino acid sequence of SEQ ID NO: 27, and a VL region comprising an amino acid sequence of SEQ ID NO: 55;
(iii) a VH region comprising an amino acid sequence of SEQ ID NO: 31, and a VL region comprising an amino acid sequence of SEQ ID NO: 55;
(iv) a VH region comprising an amino acid sequence of SEQ ID NO: 35, and a VL region comprising an amino acid sequence of SEQ ID NO: 55;
(v) a VH region comprising an amino acid sequence of SEQ ID NO: 39, and a VL region comprising an amino acid sequence of SEQ ID NO: 59;
(vi) a VH region comprising an amino acid sequence of SEQ ID NO: 43, and a VL region comprising an amino acid sequence of SEQ ID NO: 63; or
(vii) a VH region comprising an amino acid sequence of SEQ ID NO: 47, and a VL region comprising an amino acid sequence of SEQ ID NO: 67.

56.-61. (canceled)

62. The antibody or antigen binding fragment thereof of claim 1, wherein the antibody or antigen binding fragment thereof comprises:
(1) (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1, Lys 12, Val 20, Tyr 27, Thr 28, Phe 29, Thr 30, Arg 38, Met 48, Arg 67, Val 68, Ala 72, Ser 77, Ala 79, Met 81, Leu 83 and Val 117; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77 and Asp 87;
(2) (i) a VH region comprising an amino acid sequence of SEQ ID NO: 115 or an amino acid sequence comprising at least one amino acid residue substitution in SEQ ID NO: 115, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ala 72 with Thr, Ser 77 with Asp, Ala 79 with Val, Met 81 with Leu, Leu 83 with Phe, and Val 117 with Leu; and (ii) a VL region comprising an amino acid sequence of SEQ ID NO: 114 or an amino acid sequence comprising at least one amino acid residue substitution in SEQ ID NO: 114, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Ala, Thr 48 with Gly, Ser 51 with Gly, Trp 59 with Gly, Thr 60 with Val, Leu 77 with Ile, and Asp 87 with Ile;
(3) (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln1, Lys 12, Val 20, Tyr 27, Thr 28, Phe 29, Thr 30, Arg 38, Met 48, Arg 67, Val 68, Ile 70, Ala 72, Ser 77, Met 81, and Val 117; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8, Val 12, Phe 38, Gln 40, Ala 45, Pro 46, Arg 47, Thr 48, Ser 51, Trp 59, Thr 60, Leu 77, and Asp 87;
(4) (i) a VH region comprising an amino acid sequence of SEQ ID NO: 165 or an amino acid sequence comprising at least one amino acid residue substitution in SEQ ID NO: 165, wherein the at least one amino acid residue substitution is selected from substitutions at Gln 1 with Glu, Lys 12 with Val, Val 20 with Leu, Tyr 27 with Phe, Thr 28 with Asn, Phe 29 with Ile, Thr 30 with Lys, Arg 38 with Lys, Met 48 with Ile, Arg 67 with Lys, Val 68 with Ala, Ile 70 with Leu, Ala 72 with Thr, Ser 77 with Asn, Met 81 with Leu, and Val 117 with Leu; and (ii) a VL region comprising an amino acid sequence of SEQ ID NO: 164 or an amino acid sequence comprising at least one amino acid residue substitution in SEQ ID NO: 164, wherein the at least one amino acid residue substitution is selected from substitutions at Pro 8 with Ser, Val 12 with Thr, Phe 38 with Val, Gln 40 with Glu, Ala 45 with Leu, Pro 46 with Phe, Arg 47 with Thr, Thr 48 with Gly, Ser 51 with Gly, Trp 59 with Gly, Thr 60 with Val, Leu 77 with Ile, and Asp 87 with Ile:
(5) (i) a VH region comprising an amino acid sequence selected from SEQ ID NOs: 115, 139, 140, 141, 142, 143, 144, 145, 146, 147, 148, 149, 150, 151, 152, 153, 154, 155, 156, 157, 158, 159, 160, 161, 162 and 163, and a VL region comprising an amino acid sequence selected from SEQ ID NOs: 114, 116, 117, 118, 119, 120, 121, 122, 123, 124, 125, 126, 127, 128, 129, 130, 131, 132, 133, 134, 135, 136, 137 and 138; or
(6) (i) a VH region comprising an amino acid sequence selected from SEQ ID NOs: 165, 171, 172, 173, 174, 175, 176 and 177, and (ii) a VL region comprising an amino acid sequence selected from SEQ ID NOs: 164, 166, 167, 168, 169 and 170.

63.-67. (canceled)

68. The antibody or antigen binding fragment thereof of claim 1, wherein the antibody or antigen binding fragment thereof comprises:
(i) a VH region comprising an amino acid sequence of SEQ ID NO: 161, and a VL region comprising an amino acid sequence of SEQ ID NO: 130;
(ii) a VH region comprising an amino acid sequence of SEQ ID NO: 161, and a VL region comprising an amino acid sequence of SEQ ID NO: 136;
(iii) a VH region comprising an amino acid sequence of SEQ ID NO: 161, and a VL region comprising an amino acid sequence of SEQ ID NO: 137;
(iv) a VH region comprising an amino acid sequence of SEQ ID NO: 161, and a VL region comprising an amino acid sequence of SEQ ID NO: 138;
(v) a VH region comprising an amino acid sequence of SEQ ID NO: 176 and a VL region comprising an amino acid sequence of SEQ ID NO: 166;
(vi) a VH region comprising an amino acid sequence of SEQ ID NO: 176, and a VL region comprising an amino acid sequence of SEQ ID NO: 167;
(vii) a VH region comprising an amino acid sequence of SEQ ID NO: 174, and a VL region comprising an amino acid sequence of SEQ ID NO: 167; or
(viii) a VH region comprising an amino acid sequence of SEQ ID NO: 175, and a VL region comprising an amino acid sequence of SEQ ID NO: 167.

69.-75. (canceled)

76. The antibody or antigen binding fragment thereof of claim 1, wherein the antibody is a humanized antibody.

77. The antibody or antigen binding fragment thereof of claim 1, wherein the antibody or antigen binding fragment thereof is conjugated to an agent, and wherein optionally the agent is selected from a group consisting of a cytotoxic agent, a radioisotope, a metal chelator, an enzyme, a fluorescent compound, a bioluminescent compound, and a chemiluminescent compound.

78.-79. (canceled)

80. A method of treating and/or preventing a disease or disorder comprising administering a therapeutically effective amount of the antibody or antigen binding fragment thereof of claim 1, to a subject.

81. The method of claim 80, wherein the disease or disorder is a disease or disorder mediated by IL-36α and/or IL-36γ;
(ii) the disease or disorder is an inflammatory disease or an autoimmune disease;
(iii) the disease or disorder is related to skin tissue, intestinal tissue and/or lung tissue;
(iv) the disease or disorder is selected from a group consisting of generalized pustular psoriasis, palmoplantar pustulosis, palmoplantar pustular psoriasis, discoid lupus erythematosus, lupus erythematosus, atopic dermatitis, Crohn's disease, ulcerative colitis, asthma, inflammatory bowel diseases, psoriasis vulgaris, acrodermatitis continua of Hallopeau, acute generalized exanthematous pustulosis, hidradenitis suppurativa, lichen planus, Sjögren's syndrome, rheumatoid arthritis, psoriatic arthritis, chronic rhinosinusitis, acne vulgaris, impetigo herpetiformis, pyoderma gangrenosum, and polymorphic light eruption; and/or
(v) the subject is a human subject.

82.-85. (canceled)

86. A polynucleotide comprising nucleotide sequences encoding the antibody or antigen binding fragment thereof of claim 1 or a portion thereof.

87.-101. (canceled)

102. A vector comprising the polynucleotide of claim 86.

103. A cell comprising the polynucleotide of claim 86.

104.-105. (canceled)

106. A hybridoma which generates the antibody or the antibody fragment thereof of claim 1.

107. A method of making an antibody or antigen binding fragment thereof comprising culturing the cell of claim 103 to express the antibody or antigen binding fragment thereof.


SEQUENCE LISTING (177 sequences)

SEQ ID NO: 1 - protein, 158 aa, Homo sapiens
  hIL36a(Q12) (full length)
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 2 - protein, 157 aa, Homo sapiens
  hIL36b (full length)
MNPQREAAPKSYAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 3 - protein, 169 aa, Homo sapiens
  hIL36g (full length)
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 4 - DNA, 462 bp, Homo sapiens
  hIL36a(R12) (truncated)
aaaattgacacacctcagcgggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 5 - protein, 153 aa, Homo sapiens
  hIL36a(R12) (truncated)
KIDTPQRGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 6 - DNA, 462 bp, Homo sapiens
  hIL36a(Q12) (truncated)
aaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 7 - protein, 153 aa, Homo sapiens
  hIL36a(Q12) (truncated)
KIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 8 - DNA, 462 bp, Homo sapiens
  hIL36b (truncated)
cgggaggcagcacccaaatcctatgctattcgtgattctcgacagatggtgtgggtcctgagtggaaattctttaatagcagctcctcttagccgcagcattaagcctgtcactcttcatttaatagcctgtagagacacagaattcagtgacaaggaaaagggtaatatggtttacctgggaatcaagggaaaagatctctgtctcttctgtgcagaaattcagggcaagcctactttgcagcttaaggaaaaaaatatcatggacctgtatgtggagaagaaagcacagaagccctttctctttttccacaataaagaaggctccacttctgtctttcagtcagtctcttaccctggctggttcatagccacctccaccacatcaggacagcccatctttctcaccaaggagagaggcataactaataacactaacttctacttagattctgtggaataa

SEQ ID NO: 9 - protein, 153 aa, Homo sapiens
  hIL36b (truncated)
REAAPKSYAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 10 - protein, 152 aa, Homo sapiens
  hIL36g (truncated)
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 11 - protein, 154 aa, Homo sapiens
  hIL36Ra (truncated)
VLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 12 - DNA, 462 bp, Macaca fascicularis
  cyIL36a (truncated)
aaaagtgaaatgcctcagccggtgagcattcaggatatcaatcatcgggtgtgggttcttcaggaccagatcctcatagcagtcccgaggaaggaccgtgtgtctccagtcactatttccttaatctcatgccgacatgtggagacccttgagaaagacagagggaaccccatctacctgggactgaatgggctcaatctctgcttgatgtgtgctaaggccggggaccagcccacactgcagctgaaggaaaaggatataatggatttgtacaaccaacctgagcctgtgaagtcctttctcttctaccacagccagagtggcaggaactccaccttcgagtctgtggccttccctggctggttcattgctgtcagctctgaaggaggctgtcctctcatccttacccaagaactggggaaagccaacactactgactttgggttaactatgctgttttaa

SEQ ID NO: 13 - protein, 153 aa, Macaca fascicularis
  cyIL36a (truncated)
KSEMPQPVSIQDINHRVWVLQDQILIAVPRKDRVSPVTISLISCRHVETLEKDRGNPIYLGLNGLNLCLMCAKAGDQPTLQLKEKDIMDLYNQPEPVKSFLFYHSQSGRNSTFESVAFPGWFIAVSSEGGCPLILTQELGKANTTDFGLTMLF

SEQ ID NO: 14 - DNA, 462 bp, Macaca fascicularis
  cyIL36b (truncated)
tggcaggcagcacccaaatcctatgctattcgtgattctcgacagatggtgtgggtcctgagtggaaattctttaatagcagctcctcttagcaaccgtgttaagcctgtcactcttcatttaataacctgcagagacacagaattcagtgataagaaaaagggtaatctggtttacctgggaatcaggggaaaagatctctgtctcttctgtgaagaaattcagggcaaacctactttgcagcttaaggagaaaaacatcatggacctgtacatggagaagaaagcacagaagccctttctctttttccacaataaagaaggctccagttctgtctttcagtcagtctcttaccctggctggttcatagccacctcctccacatcaggacagcccatctttctcacccaggagaggggcataactaacaacactaacttctacttagattctgtggaataa

SEQ ID NO: 15 - protein, 153 aa, Macaca fascicularis
  cyIL36b (truncated)
WQAAPKSYAIRDSRQMVWVLSGNSLIAAPLSNRVKPVTLHLITCRDTEFSDKKKGNLVYLGIRGKDLCLFCEEIQGKPTLQLKEKNIMDLYMEKKAQKPFLFFHNKEGSSSVFQSVSYPGWFIATSSTSGQPIFLTQERGITNNTNFYLDSVE

SEQ ID NO: 16 - DNA, 456 bp, Macaca fascicularis
  cyIL36g (truncated)
tcaatgcgtacacctattactgggactattaatgatttgaatcagcaagtgtggacccttcagggtcagatccttgtggcagttccacgaagtgacagtgtgaccccagtcactgtcgctgttatcacatgcaagtatccagaggctcttgaccaaagcagaggggatcccatttatttgggaatccggaatccagaaatgtgtttgtgttgtgaggaggttggaggacagcccacgttgcagctaaaagagcagaagatcatggatttgtatggccagcccgagcctgtgaaacccttccttttctaccgtgtcaagaccggtaggacctccacccttgagtctgtggccttcccaaactggttcattgcctcttccacgagagaccagcccatcatcctgacttcagaacttgggaagtcatacaacactgcctttgaattaaatataaaataa

SEQ ID NO: 17 - protein, 151 aa, Macaca fascicularis
  cyIL36g (truncated)
SMRTPITGTINDLNQQVWTLQGQILVAVPRSDSVTPVTVAVITCKYPEALDQSRGDPIYLGIRNPEMCLCCEEVGGQPTLQLKEQKIMDLYGQPEPVKPFLFYRVKTGRTSTLESVAFPNWFIASSTRDQPIILTSELGKSYNTAFELNIK

SEQ ID NO: 18 - DNA, 321 bp, Artificial Sequence
  His-SUMO tag
atgcatcatcaccaccatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcaccgcgaacagattggaggt

SEQ ID NO: 19 - protein, 107 aa, Artificial Sequence
  His-SUMO tag
MHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 20 - DNA, 420 bp, Mouse
  144D464A-VH with signal peptide
atgaaatgcagctgggttatcttcttcctgatggcagtggttacaggggtcaattctgaggttcagctgcagcagtctggggcagagcttgtgaagccaggggcctcagtcaggttgtcctgcacagcttctggcttcaacattaaagacacctatatacactgggtgaagcagaggcctgaacagggcctggagtggattggaaggattgatcctgcgattggtactactagatatgacccgaagttccagggcaaggccactataacaacagacacatcctccgacacagtccacctgcagttcagcagcctgacatctgaggacactgccgtctattactgtgctagattgcactacttcggtaataacttcttctttgactactggggccaaggcaccactctcacagtctcctca

SEQ ID NO: 21 - protein, 140 aa, Mouse
  144D464A-VH with signal peptide
MKCSWVIFFLMAVVTGVNSEVQLQQSGAELVKPGASVRLSCTASGFNIKDTYIHWVKQRPEQGLEWIGRIDPAIGTTRYDPKFQGKATITTDTSSDTVHLQFSSLTSEDTAVYYCARLHYFGNNFFFDYWGQGTTLTVSS

SEQ ID NO: 22 - DNA, 363 bp, Mouse
  144D464A-VH without signal peptide
gaggttcagctgcagcagtctggggcagagcttgtgaagccaggggcctcagtcaggttgtcctgcacagcttctggcttcaacattaaagacacctatatacactgggtgaagcagaggcctgaacagggcctggagtggattggaaggattgatcctgcgattggtactactagatatgacccgaagttccagggcaaggccactataacaacagacacatcctccgacacagtccacctgcagttcagcagcctgacatctgaggacactgccgtctattactgtgctagattgcactacttcggtaataacttcttctttgactactggggccaaggcaccactctcacagtctcctca

SEQ ID NO: 23 - protein, 121 aa, Mouse
  144D464A-VH without signal peptide
EVQLQQSGAELVKPGASVRLSCTASGFNIKDTYIHWVKQRPEQGLEWIGRIDPAIGTTRYDPKFQGKATITTDTSSDTVHLQFSSLTSEDTAVYYCARLHYFGNNFFFDYWGQGTTLTVSS

SEQ ID NO: 24 - DNA, 420 bp, Mouse
  144L249B-VH with signal peptide
atgaaatgcagcggggttatcttcttcctgatggcagtggttacaggggtcaactcagaggttcagctgcagcagtctggggcagagcttgtgaagccaggggcctcagtcaagttgtcctgcacagcttctggcttcaacattaaagacacctatatatactgggtgaagcagaggcctgaacagggcctggagtggattggaaggattgatcctgcgattggtactactagatatgacccgaagttccagggcaaggccactctaacagcagacacatcctccaacacagccttcctgcagctcagcagcctgacatctgaggacactgccgtctattactgtgctagatatgactactccggtagtagcttctactttgactactggggccgaggcaccactctcacagtctcctca

SEQ ID NO: 25 - protein, 140 aa, Mouse
  144L249B-VH with signal peptide
MKCSGVIFFLMAVVTGVNSEVQLQQSGAELVKPGASVKLSCTASGFNIKDTYIYWVKQRPEQGLEWIGRIDPAIGTTRYDPKFQGKATLTADTSSNTAFLQLSSLTSEDTAVYYCARYDYSGSSFYFDYWGRGTTLTVSS

SEQ ID NO: 26 - DNA, 363 bp, Mouse
  144L249B-VH without signal peptide
gaggttcagctgcagcagtctggggcagagcttgtgaagccaggggcctcagtcaagttgtcctgcacagcttctggcttcaacattaaagacacctatatatactgggtgaagcagaggcctgaacagggcctggagtggattggaaggattgatcctgcgattggtactactagatatgacccgaagttccagggcaaggccactctaacagcagacacatcctccaacacagccttcctgcagctcagcagcctgacatctgaggacactgccgtctattactgtgctagatatgactactccggtagtagcttctactttgactactggggccgaggcaccactctcacagtctcctca

SEQ ID NO: 27 - protein, 121 aa, Mouse
  144L249B-VH without signal peptide
EVQLQQSGAELVKPGASVKLSCTASGFNIKDTYIYWVKQRPEQGLEWIGRIDPAIGTTRYDPKFQGKATLTADTSSNTAFLQLSSLTSEDTAVYYCARYDYSGSSFYFDYWGRGTTLTVSS

SEQ ID NO: 28 - DNA, 420 bp, Mouse
  144L124B, 144L180A-VH with signal peptide
atgaaatgcagctgggttatcttcttcctgatggcagtggttacaggggtcaattcagaggttcagctgcagcagtctggggcagagcttgtgaagccaggggcctcagtcaagttgtcctgcacagcttctggcttcaacattaaagacacctatatttactgggtgaagcagaggcctgaacagggcctggaatggattggaaggattgatcctgcgaatggttatactagatatgacccgaagttccagggcaaggccactatgacagcagacacatcctccaacacagcctacctgcagctcagcagcctgacatctgaggacactgccgtctattactgtgctagatatgaatactacgatagtagcttctactttgactactggggccaaggcaccactctcacagtctcctca

SEQ ID NO: 29 - protein, 140 aa, Mouse
  144L124B, 144L180A-VH with signal peptide
MKCSWVIFFLMAVVTGVNSEVQLQQSGAELVKPGASVKLSCTASGFNIKDTYIYWVKQRPEQGLEWIGRIDPANGYTRYDPKFQGKATMTADTSSNTAYLQLSSLTSEDTAVYYCARYEYYDSSFYFDYWGQGTTLTVSS

SEQ ID NO: 30 - DNA, 363 bp, Mouse
  144L124B, 144L180A-VH without signal peptide
gaggttcagctgcagcagtctggggcagagcttgtgaagccaggggcctcagtcaagttgtcctgcacagcttctggcttcaacattaaagacacctatatttactgggtgaagcagaggcctgaacagggcctggaatggattggaaggattgatcctgcgaatggttatactagatatgacccgaagttccagggcaaggccactatgacagcagacacatcctccaacacagcctacctgcagctcagcagcctgacatctgaggacactgccgtctattactgtgctagatatgaatactacgatagtagcttctactttgactactggggccaaggcaccactctcacagtctcctca

SEQ ID NO: 31 - protein, 121 aa, Mouse
  144L124B, 144L180A-VH without signal peptide
EVQLQQSGAELVKPGASVKLSCTASGFNIKDTYIYWVKQRPEQGLEWIGRIDPANGYTRYDPKFQGKATMTADTSSNTAYLQLSSLTSEDTAVYYCARYEYYDSSFYFDYWGQGTTLTVSS

SEQ ID NO: 32 - DNA, 420 bp, Mouse
  144L133B-VH with signal peptide
atgaagtgcagcggggttatcttcttcctgatggcagtggttacaggggtcaactcagaggttcagctgcagcagtctggggcagagcttgtgaagccaggggcctcagtcaagttgtcctgcacagcttctggcttcaacattaaagacacctatatgtactgggtgaagcagaggcctgaacagggcctggagtggattggaaggattgatcctgcgattggtactactagatatgacccgaagttccagggcaaggccactctaacagcagacacatcctccaacacagccttcctgcagctcagcagcctgacatctgaggacactgccgtctattactgtgctagatatgactactccggtagtagcttctactttgactactggggccgaggcaccactctcacagtctcctca

SEQ ID NO: 33 - protein, 140 aa, Mouse
  144L133B-VH with signal peptide
MKCSGVIFFLMAVVTGVNSEVQLQQSGAELVKPGASVKLSCTASGFNIKDTYMYWVKQRPEQGLEWIGRIDPAIGTTRYDPKFQGKATLTADTSSNTAFLQLSSLTSEDTAVYYCARYDYSGSSFYFDYWGRGTTLTVSS

SEQ ID NO: 34 - DNA, 363 bp, Mouse
  144L133B-VH without signal peptide
gaggttcagctgcagcagtctggggcagagcttgtgaagccaggggcctcagtcaagttgtcctgcacagcttctggcttcaacattaaagacacctatatgtactgggtgaagcagaggcctgaacagggcctggagtggattggaaggattgatcctgcgattggtactactagatatgacccgaagttccagggcaaggccactctaacagcagacacatcctccaacacagccttcctgcagctcagcagcctgacatctgaggacactgccgtctattactgtgctagatatgactactccggtagtagcttctactttgactactggggccgaggcaccactctcacagtctcctca

SEQ ID NO: 35 - protein, 121 aa, Mouse
  144L133B-VH without signal peptide
EVQLQQSGAELVKPGASVKLSCTASGFNIKDTYMYWVKQRPEQGLEWIGRIDPAIGTTRYDPKFQGKATLTADTSSNTAFLQLSSLTSEDTAVYYCARYDYSGSSFYFDYWGRGTTLTVSS

SEQ ID NO: 36 - DNA, 420 bp, Mouse
  144L472A-VH with signal peptide
atgaaatgcagctgggttatcttcttcctgatggcagtggttacaggggtcaattcagaggttcagctgcagcagtctggggcagagcttgtgaagccaggggcctcagtcaagttgtcctgcacagtttctggcttcaacattaaagacacctatatgtactgggtgaagcagaggcctgaacagggcctggagtggattggaaggattgatcctgcgaatggttatgctaaatatgacccgaagttccagggcaaggccactttaacagcagacacatcttccaatacagcctacctgcagctcagcagcctgacatctgaggacactgccgtctatttctgtgctagatttcattggtacgacagtgccttctactttgacttctggggccaaggcaccactctcacagtctcctca

SEQ ID NO: 37 - protein, 140 aa, Mouse
  144L472A-VH with signal peptide
MKCSWVIFFLMAVVTGVNSEVQLQQSGAELVKPGASVKLSCTVSGFNIKDTYMYWVKQRPEQGLEWIGRIDPANGYAKYDPKFQGKATLTADTSSNTAYLQLSSLTSEDTAVYFCARFHWYDSAFYFDFWGQGTTLTVSS

SEQ ID NO: 38 - DNA, 363 bp, Mouse
  144L472A-VH without signal peptide
gaggttcagctgcagcagtctggggcagagcttgtgaagccaggggcctcagtcaagttgtcctgcacagtttctggcttcaacattaaagacacctatatgtactgggtgaagcagaggcctgaacagggcctggagtggattggaaggattgatcctgcgaatggttatgctaaatatgacccgaagttccagggcaaggccactttaacagcagacacatcttccaatacagcctacctgcagctcagcagcctgacatctgaggacactgccgtctatttctgtgctagatttcattggtacgacagtgccttctactttgacttctggggccaaggcaccactctcacagtctcctca

SEQ ID NO: 39 - protein, 121 aa, Mouse
  144L472A-VH without signal peptide
EVQLQQSGAELVKPGASVKLSCTVSGFNIKDTYMYWVKQRPEQGLEWIGRIDPANGYAKYDPKFQGKATLTADTSSNTAYLQLSSLTSEDTAVYFCARFHWYDSAFYFDFWGQGTTLTVSS

SEQ ID NO: 40 - DNA, 420 bp, Mouse
  144D666C-VH with signal peptide
atgaaatgcagctgggttatcttcttcctgatggcagtggttacaggggtcaattcagaggttcagctgcagcagtctggggcagagcttgtgaagccaggggcctcagtcaagttgacctgcacagcttctggcttcaacattaaagacacctatatatactgggtgaaacagaggcctgcacagggcctggagtggattggaaggattgatcctgcgaatgcttatactaaatttgacccgaagttccagggcaaggccactttaacagcagacacatcctccaacacagcctacctgcagctcagcagcctgacatctgaggacactgccgtctattactgtactagatttcattggtacggtagtagcttcttctttgactactggggccaaggcaccactctcacagtctcctca

SEQ ID NO: 41 - protein, 140 aa, Mouse
  144D666C-VH with signal peptide
MKCSWVIFFLMAVVTGVNSEVQLQQSGAELVKPGASVKLTCTASGFNIKDTYIYWVKQRPAQGLEWIGRIDPANAYTKFDPKFQGKATLTADTSSNTAYLQLSSLTSEDTAVYYCTRFHWYGSSFFFDYWGQGTTLTVSS

SEQ ID NO: 42 - DNA, 363 bp, Mouse
  144D666C-VH without signal peptide
gaggttcagctgcagcagtctggggcagagcttgtgaagccaggggcctcagtcaagttgacctgcacagcttctggcttcaacattaaagacacctatatatactgggtgaaacagaggcctgcacagggcctggagtggattggaaggattgatcctgcgaatgcttatactaaatttgacccgaagttccagggcaaggccactttaacagcagacacatcctccaacacagcctacctgcagctcagcagcctgacatctgaggacactgccgtctattactgtactagatttcattggtacggtagtagcttcttctttgactactggggccaaggcaccactctcacagtctcctca

SEQ ID NO: 43 - protein, 121 aa, Mouse
  144D666C-VH without signal peptide
EVQLQQSGAELVKPGASVKLTCTASGFNIKDTYIYWVKQRPAQGLEWIGRIDPANAYTKFDPKFQGKATLTADTSSNTAYLQLSSLTSEDTAVYYCTRFHWYGSSFFFDYWGQGTTLTVSS

SEQ ID NO: 44 - DNA, 420 bp, Mouse
  144J171G-VH with signal peptide
atgaaatgcagctgggttatcttcttcctgatggcagtggttacaggggtctattctgaggttcagctgcagcagtctggggcagagcttgtggagccaggggcctcagtcaagttgtcctgcacagcttctggcttcaacattaaagacacctacataaactgggtgaagcagaggcctgaacagggcctggagtggattggaaggattgatcctgcgaatggttatactagatatgccccgaagttccagggcaaggccactataacatcagacacatcctccaacacagcctacctgcagctcagcagcctgacatctgaggacgctgccgtctattcctgttctacattaaattactacggtagtagctttttctttgacttctggggccaaggcaccactctcacagtctcctca

SEQ ID NO: 45 - protein, 140 aa, Mouse
  144J171G-VH with signal peptide
MKCSWVIFFLMAVVTGVYSEVQLQQSGAELVEPGASVKLSCTASGFNIKDTYINWVKQRPEQGLEWIGRIDPANGYTRYAPKFQGKATITSDTSSNTAYLQLSSLTSEDAAVYSCSTLNYYGSSFFFDFWGQGTTLTVSS

SEQ ID NO: 46 - DNA, 363 bp, Mouse
  144J171G-VH without signal peptide
gaggttcagctgcagcagtctggggcagagcttgtggagccaggggcctcagtcaagttgtcctgcacagcttctggcttcaacattaaagacacctacataaactgggtgaagcagaggcctgaacagggcctggagtggattggaaggattgatcctgcgaatggttatactagatatgccccgaagttccagggcaaggccactataacatcagacacatcctccaacacagcctacctgcagctcagcagcctgacatctgaggacgctgccgtctattcctgttctacattaaattactacggtagtagctttttctttgacttctggggccaaggcaccactctcacagtctcctca

SEQ ID NO: 47 - protein, 121 aa, Mouse
  144J171G-VH without signal peptide
EVQLQQSGAELVEPGASVKLSCTASGFNIKDTYINWVKQRPEQGLEWIGRIDPANGYTRYAPKFQGKATITSDTSSNTAYLQLSSLTSEDAAVYSCSTLNYYGSSFFFDFWGQGTTLTVSS

SEQ ID NO: 48 - DNA, 384 bp, Mouse
  144D464A-VL with signal peptide
atggcctggatttcacttatactctctctcctggctctcagctcaggggccatttcccaggctgttgtgactcaggaatctgcactcaccacatcacctggtgaagcagtcacactcacttgtcgctcaagttctggggctgttacaactagtaactatgccaactgggtccaagaaaaaccagatcatttattcgctggtctaataggtggtaccaacgaccgagctccaggtgttcctgccagattctcaggctccctgattggagacaaggctgccctcaccatcacaggggcacagactgaggatgaggcaatatatttctgtgctctatggttcagcaaccattgggtgttcggtggaggaaccaaactgactgtccta

SEQ ID NO: 49 - protein, 128 aa, Mouse
  144D464A-VL with signal peptide
MAWISLILSLLALSSGAISQAVVTQESALTTSPGEAVTLTCRSSSGAVTTSNYANWVQEKPDHLFAGLIGGTNDRAPGVPARFSGSLIGDKAALTITGAQTEDEAIYFCALWFSNHWVFGGGTKLTVL

SEQ ID NO: 50 - DNA, 327 bp, Mouse
  144D464A-VL without signal peptide
caggctgttgtgactcaggaatctgcactcaccacatcacctggtgaagcagtcacactcacttgtcgctcaagttctggggctgttacaactagtaactatgccaactgggtccaagaaaaaccagatcatttattcgctggtctaataggtggtaccaacgaccgagctccaggtgttcctgccagattctcaggctccctgattggagacaaggctgccctcaccatcacaggggcacagactgaggatgaggcaatatatttctgtgctctatggttcagcaaccattgggtgttcggtggaggaaccaaactgactgtccta

SEQ ID NO: 51 - protein, 109 aa, Mouse
  144D464A-VL without signal peptide
QAVVTQESALTTSPGEAVTLTCRSSSGAVTTSNYANWVQEKPDHLFAGLIGGTNDRAPGVPARFSGSLIGDKAALTITGAQTEDEAIYFCALWFSNHWVFGGGTKLTVL

SEQ ID NO: 52 - DNA, 384 bp, Mouse
  144L249B, 144L124B, 144L180A, 144L133B-VL with signal peptide
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtccta

SEQ ID NO: 53 - protein, 128 aa, Mouse
  144L249B, 144L124B, 144L180A, 144L133B-VL with signal peptide
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 54 - DNA, 327 bp, Mouse
  144L249B, 144L124B, 144L180A, 144L133B-VL without signal peptide
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtccta

SEQ ID NO: 55 - protein, 109 aa, Mouse
  144L249B, 144L124B, 144L180A, 144L133B-VL without signal peptide
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 56 - DNA, 384 bp, Mouse
  144L472A-VL with signal peptide
atggcctggatttcacttatactctctctcctggctctcagctcaggggccatttcccaggctgttgtgactcaggaatctgcactcaccacatcacctggtgaaacagtcacactcacttgtcgctcaagtagtggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtggtctatggtacagcaaccattgggtgttcggtggaggaaccaaactgactgtccta

SEQ ID NO: 57 - protein, 128 aa, Mouse
  144L472A-VL with signal peptide
MAWISLILSLLALSSGAISQAVVTQESALTTSPGETVTLTCRSSSGAVTTSNYANWVQEKPDHLFTGLIGGTNNRAPGVPARFSGSLIGDKAALTITGAQTEDEAIYFCGLWYSNHWVFGGGTKLTVL

SEQ ID NO: 58 - DNA, 327 bp, Mouse
  144L472A-VL without signal peptide
caggctgttgtgactcaggaatctgcactcaccacatcacctggtgaaacagtcacactcacttgtcgctcaagtagtggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtggtctatggtacagcaaccattgggtgttcggtggaggaaccaaactgactgtccta

SEQ ID NO: 59 - protein, 109 aa, Mouse
  144L472A-VL without signal peptide
QAVVTQESALTTSPGETVTLTCRSSSGAVTTSNYANWVQEKPDHLFTGLIGGTNNRAPGVPARFSGSLIGDKAALTITGAQTEDEAIYFCGLWYSNHWVFGGGTKLTVL

SEQ ID NO: 60 - DNA, 384 bp, Mouse
  144D666C-VL with signal peptide
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttgttcactggtctaataggtggtaccgacaaccgacctccaggtgttcctgccagattctcaggctccctgattggagacaaggctgccctcaccatcacaggggcacagactgaggatgaggcaatttatttctgtgctctatggtacagcaacctctgggtgttcggtggaggaaccaaactgactgtccta

SEQ ID NO: 61 - protein, 128 aa, Mouse
  144D666C-VL with signal peptide
MAWISLILSLLALSSGAISQAVVTQESALTTSPGETVTLTCRSSTGAVTTSNYANWVQEKPDHLFTGLIGGTDNRPPGVPARFSGSLIGDKAALTITGAQTEDEAIYFCALWYSNLWVFGGGTKLTVL

SEQ ID NO: 62 - DNA, 327 bp, Mouse
  144D666C-VL without signal peptide
caggctgttgtgactcaggaatctgcactcaccacatcacctggtgaaacagtcacactcacttgtcgctcaagtactggggctgttacaactagtaactatgccaactgggtccaagaaaaaccagatcatttgttcactggtctaataggtggtaccgacaaccgacctccaggtgttcctgccagattctcaggctccctgattggagacaaggctgccctcaccatcacaggggcacagactgaggatgaggcaatttatttctgtgctctatggtacagcaacctctgggtgttcggtggaggaaccaaactgactgtccta

SEQ ID NO: 63 - protein, 109 aa, Mouse
  144D666C-VL without signal peptide
QAVVTQESALTTSPGETVTLTCRSSTGAVTTSNYANWVQEKPDHLFTGLIGGTDNRPPGVPARFSGSLIGDKAALTITGAQTEDEAIYFCALWYSNLWVFGGGTKLTVL

SEQ ID NO: 64 - DNA, 384 bp, Mouse
  144J171G-VL with signal peptide
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 65 - protein, 128 aa, Mouse
  144J171G-VL with signal peptide
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 66 - DNA, 327 bp, Mouse
  144J171G-VL without signal peptide
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 67 - protein, 109 aa, Mouse
  144J171G-VL without signal peptide
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 68 - protein, 5 aa, Mouse
  144D464A CDRH1
DTYIH

SEQ ID NO: 69 - protein, 17 aa, Mouse
  144D464A,144L249B, 144L133B CDRH2
RIDPAIGTTRYDPKFQG

SEQ ID NO: 70 - protein, 12 aa, Mouse
  144D464A CDRH3
LHYFGNNFFFDY

SEQ ID NO: 71 - protein, 5 aa, Mouse
  144L249B, 144L124B, 144L180A, 144D666C CDRH1
DTYIY

SEQ ID NO: 72 - protein, 12 aa, Mouse
  144L249B, 144L133B CDRH3
YDYSGSSFYFDY

SEQ ID NO: 73 - protein, 17 aa, Mouse
  144L124B, 144L180A CDRH2
RIDPANGYTRYDPKFQG

SEQ ID NO: 74 - protein, 12 aa, Mouse
  144L124B, 144L180A CDRH3
YEYYDSSFYFDY

SEQ ID NO: 75 - protein, 5 aa, Mouse
  144L133B, 144L472A CDRH1
DTYMY

SEQ ID NO: 76 - protein, 17 aa, Mouse
  144L472A CDRH2
RIDPANGYAKYDPKFQG

SEQ ID NO: 77 - protein, 12 aa, Mouse
  144L472A CDRH3
FHWYDSAFYFDF

SEQ ID NO: 78 - protein, 17 aa, Mouse
  144D666C CDRH2
RIDPANAYTKFDPKFQG

SEQ ID NO: 79 - protein, 12 aa, Mouse
  144D666C CDRH3
FHWYGSSFFFDY

SEQ ID NO: 80 - protein, 5 aa, Mouse
  144J171G CDRH1
DTYIN

SEQ ID NO: 81 - protein, 17 aa, Mouse
  144J171G CDRH2
RIDPANGYTRYAPKFQG

SEQ ID NO: 82 - protein, 12 aa, Mouse
  144J171G CDRH3
LNYYGSSFFFDF

SEQ ID NO: 83 - protein, 14 aa, Mouse
  144D464A, 144L472A CDRL1
RSSSGAVTTSNYAN

SEQ ID NO: 84 - protein, 7 aa, Mouse
  144D464A CDRL2
GTNDRAP

SEQ ID NO: 85 - protein, 9 aa, Mouse
  144D464A CDRL3
ALWFSNHWV

SEQ ID NO: 86 - protein, 14 aa, Mouse
  144L249B, 144L124B, 144L180A, 144L133B, 144D666C, 144J171G CDRL1
RSSTGAVTTSNYAN

SEQ ID NO: 87 - protein, 7 aa, Mouse
  144L249B, 144L124B, 144L180A, 144L133B, 144L472A, 144J171G CDRL2
GTNNRAP

SEQ ID NO: 88 - protein, 9 aa, Mouse
  144L249B, 144L124B, 144L180A, 144L133B CDRL3
ALWYSNHLV

SEQ ID NO: 89 - protein, 9 aa, Mouse
  144L472A CDRL3
GLWYSNHWV

SEQ ID NO: 90 - protein, 7 aa, Mouse
  144D666C CDRL2
GTDNRPP

SEQ ID NO: 91 - protein, 9 aa, Mouse
  144D666C CDRL3
ALWYSNLWV

SEQ ID NO: 92 - protein, 9 aa, Mouse
  144J171G CDRL3
ALWYSNHWV

SEQ ID NO: 93 - DNA, 993 bp, Homo sapiens
  IgG1 constant (human)
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 94 - protein, 330 aa, Homo sapiens [sequence omitted]

SEQ ID NO: 95 - DNA, 321 bp, Homo sapiens
  IgLamdba constant (human) IGLC2
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatga

SEQ ID NO: 96 - protein, 106 aa, Homo sapiens
  IgLamdba constant (human) IGLC2
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - DNA, 36 bp, Artificial Sequence
  primer_1262
gattacgccaagcttgtcactggctcagggaaataa

SEQ ID NO: 98 - DNA, 38 bp, Artificial Sequence
  primer_1267
  misc_feature (19)..(19): n is c or t
  misc_feature (24)..(24): n is a or g
  misc_feature (34)..(34): n is a or g
gattacgccaagcttctcntcagnggaaggtggnaaca

SEQ ID NO: 99 - DNA, 474 bp, Homo sapiens
  hIL-36a(Q12) full length
atggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttt

SEQ ID NO: 100 - DNA, 474 bp, Homo sapiens
  hIL-36a(R12) full length
atggaaaaagcattgaaaattgacacacctcagcgggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttt

SEQ ID NO: 101 - protein, 158 aa, Homo sapiens
  hIL-36a(R12) full length
MEKALKIDTPQRGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 102 - DNA, 471 bp, Homo sapiens
  hIL-36b full length
atgaacccacaacgggaggcagcacccaaatcctatgctattcgtgattctcgacagatggtgtgggtcctgagtggaaattctttaatagcagctcctcttagccgcagcattaagcctgtcactcttcatttaatagcctgtagagacacagaattcagtgacaaggaaaagggtaatatggtttacctgggaatcaagggaaaagatctctgtctcttctgtgcagaaattcagggcaagcctactttgcagcttaaggaaaaaaatatcatggacctgtatgtggagaagaaagcacagaagccctttctctttttccacaataaagaaggctccacttctgtctttcagtcagtctcttaccctggctggttcatagccacctccaccacatcaggacagcccatctttctcaccaaggagagaggcataactaataacactaacttctacttagattctgtggaa

SEQ ID NO: 103 - DNA, 507 bp, Homo sapiens
  hIL-36g full length
atgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgac

SEQ ID NO: 104 - DNA, 456 bp, Homo sapiens
  hIL-36g truncated
tcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgac

SEQ ID NO: 105 - DNA, 465 bp, Homo sapiens
  hIL-36Ra full length
atggtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgac

SEQ ID NO: 106 - protein, 155 aa, Homo sapiens
  hIL-36Ra full length
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 107 - DNA, 462 bp, Homo sapiens
  hIL-36Ra truncated
gtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgac

SEQ ID NO: 108 - DNA, 537 bp, Cyno
  cyIL-36a full length
atgaaaaaattcattgttgtactatatggaaaactcaggctgtgttcatggtctttgagtgaactattttcaatgtcgaaaagtgaaatgcctcagccggtgagcattcaggatatcaatcatcgggtgtgggttcttcaggaccagatcctcatagcagtcccgaggaaggaccgtgtgtctccagtcactatttccttaatctcatgccgacatgtggagacccttgagaaagacagagggaaccccatctacctgggactgaatgggctcaatctctgcttgatgtgtgctaaggccggggaccagcccacactgcagctgaaggaaaaggatataatggatttgtacaaccaacctgagcctgtgaagtcctttctcttctaccacagccagagtggcaggaactccaccttcgagtctgtggccttccctggctggttcattgctgtcagctctgaaggaggctgtcctctcatccttacccaagaactggggaaagccaacactactgactttgggttaactatgctgttt

SEQ ID NO: 109 - protein, 179 aa, Cyno
  cyIL-36a full length
MKKFIVVLYGKLRLCSWSLSELFSMSKSEMPQPVSIQDINHRVWVLQDQILIAVPRKDRVSPVTISLISCRHVETLEKDRGNPIYLGLNGLNLCLMCAKAGDQPTLQLKEKDIMDLYNQPEPVKSFLFYHSQSGRNSTFESVAFPGWFIAVSSEGGCPLILTQELGKANTTDFGLTMLF

SEQ ID NO: 110 - DNA, 471 bp, Cyno
  cyIL-36b full length
atgaacccacaatggcaggcagcacccaaatcctatgctattcgtgattctcgacagatggtgtgggtcctgagtggaaattctttaatagcagctcctcttagcaaccgtgttaagcctgtcactcttcatttaataacctgcagagacacagaattcagtgataagaaaaagggtaatctggtttacctgggaatcaggggaaaagatctctgtctcttctgtgaagaaattcagggcaaacctactttgcagcttaaggagaaaaacatcatggacctgtacatggagaagaaagcacagaagccctttctctttttccacaataaagaaggctccagttctgtctttcagtcagtctcttaccctggctggttcatagccacctcctccacatcaggacagcccatctttctcacccaggagaggggcataactaacaacactaacttctacttagattctgtggaa

SEQ ID NO: 111 - protein, 157 aa, Cyno
  cyIL-36b full length
MNPQWQAAPKSYAIRDSRQMVWVLSGNSLIAAPLSNRVKPVTLHLITCRDTEFSDKKKGNLVYLGIRGKDLCLFCEEIQGKPTLQLKEKNIMDLYMEKKAQKPFLFFHNKEGSSSVFQSVSYPGWFIATSSTSGQPIFLTQERGITNNTNFYLDSVE

SEQ ID NO: 112 - DNA, 504 bp, Cyno
  cyIL-36g full length
atgagaggcactccaggaaaccctgctggtggaggaagggtcgtctatcagtcaatgcgtacacctattactgggactattaatgatttgaatcagcaagtgtggacccttcagggtcagatccttgtggcagttccacgaagtgacagtgtgaccccagtcactgtcgctgttatcacatgcaagtatccagaggctcttgaccaaagcagaggggatcccatttatttgggaatccggaatccagaaatgtgtttgtgttgtgaggaggttggaggacagcccacgttgcagctaaaagagcagaagatcatggatttgtatggccagcccgagcctgtgaaacccttccttttctaccgtgtcaagaccggtaggacctccacccttgagtctgtggccttcccaaactggttcattgcctcttccacgagagaccagcccatcatcctgacttcagaacttgggaagtcatacaacactgcctttgaattaaatataaaa

SEQ ID NO: 113 - protein, 168 aa, Cyno
  cyIL-36g full length
MRGTPGNPAGGGRVVYQSMRTPITGTINDLNQQVWTLQGQILVAVPRSDSVTPVTVAVITCKYPEALDQSRGDPIYLGIRNPEMCLCCEEVGGQPTLQLKEQKIMDLYGQPEPVKPFLFYRVKTGRTSTLESVAFPNWFIASSTRDQPIILTSELGKSYNTAFELNIK

SEQ ID NO: 114 - protein, 109 aa, Artificial Sequence
  144D464A LV0
QAVVTQEPSLTVSPGGTVTLTCRSSSGAVTTSNYANWFQQKPGQAPRTLISGTNDRAPWTPARFSGSLLGGKAALTLSGAQPEDEADYFCALWFSNHWVFGGGTKLTVL

SEQ ID NO: 115 - protein, 121 aa, Artificial Sequence
  144D464A HV0
QVQLVQSGAEVKKPGASVKVSCKASGYTFTDTYIHWVRQAPGQGLEWMGRIDPAIGTTRYDPKFQGRVTITADTSTSTAYMELSSLRSEDTAVYYCARLHYFGNNFFFDYWGQGTLVTVSS

SEQ ID NO: 116 - protein, 109 aa, Artificial Sequence
  144D464A LV3a
QAVVTQEPSLTVSPGGTVTLTCRSSSGAVTTSNYANWFQQKPGQAPRGLISGTNDRAPWVPARFSGSLLGGKAALTISGAQPEDEADYFCALWFSNHWVFGGGTKLTVL

SEQ ID NO: 117 - protein, 109 aa, Artificial Sequence
  144D464A LV3b
QAVVTQEPSLTVSPGGTVTLTCRSSSGAVTTSNYANWVQQKPGQAPRGLISGTNDRAPWVPARFSGSLLGGKAALTLSGAQPEDEADYFCALWFSNHWVFGGGTKLTVL

SEQ ID NO: 118 - protein, 109 aa, Artificial Sequence
  144D464A LV4a
QAVVTQEPSLTVSPGGTVTLTCRSSSGAVTTSNYANWVQQKPGQAPRGLISGTNDRAPWVPARFSGSLLGGKAALTISGAQPEDEADYFCALWFSNHWVFGGGTKLTVL

SEQ ID NO: 119 - protein, 109 aa, Artificial Sequence
  144D464A LV4b
QAVVTQEPSLTVSPGGTVTLTCRSSSGAVTTSNYANWVQQKPGQAPRGLIGGTNDRAPWVPARFSGSLLGGKAALTLSGAQPEDEADYFCALWFSNHWVFGGGTKLTVL

SEQ ID NO: 120 - protein, 109 aa, Artificial Sequence
  144D464A LV5a
QAVVTQEPSLTVSPGGTVTLTCRSSSGAVTTSNYANWVQQKPGQAPAGLIGGTNDRAPWVPARFSGSLLGGKAALTLSGAQPEDEADYFCALWFSNHWVFGGGTKLTVL

SEQ ID NO: 121 - protein, 109 aa, Artificial Sequence
  144D464A LV5b
QAVVTQEPSLTVSPGGTVTLTCRSSSGAVTTSNYANWVQQKPGQLFATLISGTNDRAPWTPARFSGSLLGGKAALTLSGAQPEDEAIYFCALWFSNHWVFGGGTKLTVL

SEQ ID NO: 122 - protein, 109 aa, Artificial Sequence
  144D464A LV5c
QAVVTQEPSLTVSPGGTVTLTCRSSSGAVTTSNYANWVQEKPGQAPRGLISGTNDRAPWVPARFSGSLLGGKAALTISGAQPEDEADYFCALWFSNHWVFGGGTKLTVL

SEQ ID NO: 123 - protein, 109 aa, Artificial Sequence
  144D464A LV5d
QAVVTQEPSLTVSPGGTVTLTCRSSSGAVTTSNYANWVQEKPGQAFRGLISGTNDRAPWVPARFSGSLLGGKAALTLSGAQPEDEADYFCALWFSNHWVFGGGTKLTVL

SEQ ID NO: 124 - protein, 109 aa, Artificial Sequence
  144D464A LV5e
QAVVTQEPSLTTSPGGTVTLTCRSSSGAVTTSNYANWVQQKPGQAPRGLIGGTNDRAPWVPARFSGSLLGGKAALTLSGAQPEDEADYFCALWFSNHWVFGGGTKLTVL

SEQ ID NO: 125 - protein, 109 aa, Artificial Sequence
  144D464A LV6a
QAVVTQEPSLTVSPGGTVTLTCRSSSGAVTTSNYANWFQQKPGQLFAGLISGTNDRAPGTPARFSGSLLGGKAALTLSGAQPEDEAIYFCALWFSNHWVFGGGTKLTVL

SEQ ID NO: 126 - protein, 109 aa, Artificial Sequence
  144D464A LV6b
QAVVTQEPSLTVSPGGTVTLTCRSSSGAVTTSNYANWVQQKPGQAPAGLIGGTNDRAPGVPARFSGSLLGGKAALTLSGAQPEDEADYFCALWFSNHWVFGGGTKLTVL

SEQ ID NO: 127 - protein, 109 aa, Artificial Sequence
  144D464A LV6c
QAVVTQEPSLTVSPGGTVTLTCRSSSGAVTTSNYANWVQQKPGQLFAGLIGGTNDRAPWTPARFSGSLLGGKAALTLSGAQPEDEADYFCALWFSNHWVFGGGTKLTVL

SEQ ID NO: 128 - protein, 109 aa, Artificial Sequence
  144D464A LV6d
QAVVTQEPSLTTSPGGTVTLTCRSSSGAVTTSNYANWFQQKPGQAPRGLIGGTNDRAPWVPARFSGSLLGGKAALTISGAQPEDEAIYFCALWFSNHWVFGGGTKLTVL

SEQ ID NO: 129 - protein, 109 aa, Artificial Sequence
  144D464A LV6e
QAVVTQEPSLTVSPGGTVTLTCRSSSGAVTTSNYANWVQEKPGQAFRGLIGGTNDRAPWVPARFSGSLLGGKAALTLSGAQPEDEADYFCALWFSNHWVFGGGTKLTVL

SEQ ID NO: 130 - protein, 109 aa, Artificial Sequence
  144D464A LV7a
QAVVTQEPSLTVSPGGTVTLTCRSSSGAVTTSNYANWVQQKPGQAFAGLIGGTNDRAPGVPARFSGSLLGGKAALTISGAQPEDEADYFCALWFSNHWVFGGGTKLTVL

SEQ ID NO: 131 - protein, 109 aa, Artificial Sequence
  144D464A LV7b
QAVVTQESSLTTSPGGTVTLTCRSSSGAVTTSNYANWVQQKPGQAPRGLIGGTNDRAPWVPARFSGSLLGGKAALTLSGAQPEDEAIYFCALWFSNHWVFGGGTKLTVL

SEQ ID NO: 132 - protein, 109 aa, Artificial Sequence
  144D464A LV8
QAVVTQEPSLTTSPGGTVTLTCRSSSGAVTTSNYANWFQQKPGQLPAGLIGGTNDRAPWVPARFSGSLLGGKAALTISGAQPEDEAIYFCALWFSNHWVFGGGTKLTVL

SEQ ID NO: 133 - protein, 109 aa, Artificial Sequence
  144D464A LV9
QAVVTQEPSLTTSPGGTVTLTCRSSSGAVTTSNYANWVQEKPGQAFRGLIGGTNDRAPWVPARFSGSLLGGKAALTISGAQPEDEAIYFCALWFSNHWVFGGGTKLTVL

SEQ ID NO: 134 - protein, 109 aa, Artificial Sequence
  144D464A LV11
QAVVTQESSLTTSPGGTVTLTCRSSSGAVTTSNYANWVQQKPGQLFAGLIGGTNDRAPGVPARFSGSLLGGKAALTLSGAQPEDEAIYFCALWFSNHWVFGGGTKLTVL

SEQ ID NO: 135 - protein, 109 aa, Artificial Sequence
  144D464A LV12(+1)
QAVVTQESSLTTSPGGTVTLTCRSSSGAVTTSNYANWVQEKPGQLFAGLIGGTNDRAPGVPARFSGSLLGGKAALTISGAQPEDEAIYFCALWFSNHWVFGGGTKLTVL

SEQ ID NO: 136 - protein, 109 aa, Artificial Sequence
  144D464A LV9are
QAVVTQEPSLTVSPGGTVTLTCRSSSGAVTTSNYANWVQEKPGQLFAGLIGGTNDRAPGVPARFSGSLLGGKAALTLSGAQPEDEADYFCALWFSNHWVFGGGTKLTVL

SEQ ID NO: 137 - protein, 109 aa, Artificial Sequence
  144D464A LV10re
QAVVTQEPSLTVSPGGTVTLTCRSSSGAVTTSNYANWVQEKPGQLFAGLIGGTNDRAPGVPARFSGSLLGGKAALTLSGAQPEDEAIYFCALWFSNHWVFGGGTKLTVL

SEQ ID NO: 138 - protein, 109 aa, Artificial Sequence
  144D464A LV11re
QAVVTQEPSLTVSPGGTVTLTCRSSSGAVTTSNYANWVQEKPGQLFAGLIGGTNDRAPGVPARFSGSLLGGKAALTISGAQPEDEAIYFCALWFSNHWVFGGGTKLTVL

SEQ ID NO: 139 - protein, 121 aa, Artificial Sequence
  144D464A HV1
QVQLVQSGAEVKKPGASVKVSCKASGYTFTDTYIHWVRQAPGQGLEWMGRIDPAIGTTRYDPKFQGRVTITADTSTDTAYMELSSLRSEDTAVYYCARLHYFGNNFFFDYWGQGTLVTVSS

SEQ ID NO: 140 - protein, 121 aa, Artificial Sequence
  144D464A HV4a
QVQLVQSGAEVKKPGASVKVSCKASGYTFTDTYIHWVRQAPGQGLEWMGRIDPAIGTTRYDPKFQGRVTITTDTSTDTVYMEFSSLRSEDTAVYYCARLHYFGNNFFFDYWGQGTLVTVSS

SEQ ID NO: 141 - protein, 121 aa, Artificial Sequence
  144D464A HV4b
QVQLVQSGAEVKKPGASVKVSCKASGYNFKDTYIHWVRQAPGQGLEWMGRIDPAIGTTRYDPKFQGRATITADTSTSTVYMELSSLRSEDTAVYYCARLHYFGNNFFFDYWGQGTLVTVSS

SEQ ID NO: 142 - protein, 121 aa, Artificial Sequence
  144D464A HV4c
QVQLVQSGAEVKKPGASVKVSCKASGFNIKDTYIHWVRQAPGQGLEWMGRIDPAIGTTRYDPKFQGRVTITADTSTSTAYMELSSLRSEDTAVYYCARLHYFGNNFFFDYWGQGTLVTVSS

SEQ ID NO: 143 - protein, 121 aa, Artificial Sequence
  144D464A HV5a
QVQLVQSGAEVKKPGASVKVSCKASGYNFKDTYIHWVRQAPGQGLEWIGRIDPAIGTTRYDPKFQGKATITTDTSTSTAYMELSSLRSEDTAVYYCARLHYFGNNFFFDYWGQGTLVTVSS

SEQ ID NO: 144 - protein, 121 aa, Artificial Sequence
  144D464A HV5b
QVQLVQSGAEVVKPGASVKVSCKASGFTFKDTYIHWVRQAPGQGLEWIGRIDPAIGTTRYDPKFQGRVTITTDTSTSTAYMELSSLRSEDTAVYYCARLHYFGNNFFFDYWGQGTLVTVSS

SEQ ID NO: 145 - protein, 121 aa, Artificial Sequence
  144D464A HV5c
QVQLVQSGAEVVKPGASVKVSCKASGYNFKDTYIHWVRQAPGQGLEWMGRIDPAIGTTRYDPKFQGRATITADTSTSTVYMELSSLRSEDTAVYYCARLHYFGNNFFFDYWGQGTLVTVSS

SEQ ID NO: 146 - protein, 121 aa, Artificial Sequence
  144D464A HV5d
QVQLVQSGAEVKKPGASVKVSCKASGYNIKDTYIHWVRQAPGQGLEWIGRIDPAIGTTRYDPKFQGRATITADTSTSTAYLELSSLRSEDTAVYYCARLHYFGNNFFFDYWGQGTLVTVSS

SEQ ID NO: 147 - protein, 121 aa, Artificial Sequence
  144D464A HV5e
QVQLVQSGAEVKKPGASVKVSCKASGYTIKDTYIHWVRQAPGQGLEWIGRIDPAIGTTRYDPKFQGRVTITADTSTSTAYLEFSSLRSEDTAVYYCARLHYFGNNFFFDYWGQGTLVTVSS

SEQ ID NO: 148 - protein, 121 aa, Artificial Sequence
  144D464A HV5f
QVQLVQSGAEVKKPGASVKVSCKASGYNIKDTYIHWVRQAPGQGLEWIGRIDPAIGTTRYDPKFQGRATITADTSTSTAYMELSSLRSEDTAVYYCARLHYFGNNFFFDYWGQGTLVTVSS

SEQ ID NO: 149 - protein, 121 aa, Artificial Sequence
  144D464A HV5g
QVQLVQSGAEVKKPGASVKVSCKASGFTIKDTYIHWVRQAPGQGLEWMGRIDPAIGTTRYDPKFQGRATITTDTSTSTAYMELSSLRSEDTAVYYCARLHYFGNNFFFDYWGQGTLVTVSS

SEQ ID NO: 150 - protein, 121 aa, Artificial Sequence
  144D464A HV6a
QVQLVQSGAEVVKPGASVKVSCKASGYNFKDTYIHWVRQAPGQGLEWMGRIDPAIGTTRYDPKFQGRVTITTDTSTSTVYMEFSSLRSEDTAVYYCARLHYFGNNFFFDYWGQGTLVTVSS

SEQ ID NO: 151 - protein, 121 aa, Artificial Sequence
  144D464A HV6b
QVQLVQSGAEVKKPGASVKVSCKASGYNFKDTYIHWVRQAPGQGLEWMGRIDPAIGTTRYDPKFQGRATITADTSTDTVYMEFSSLRSEDTAVYYCARLHYFGNNFFFDYWGQGTLVTVSS

SEQ ID NO: 152 - protein, 121 aa, Artificial Sequence
  144D464A HV6c
QVQLVQSGAEVKKPGASVKVSCKASGYTIKDTYIHWVRQAPGQGLEWIGRIDPAIGTTRYDPKFQGRATITADTSTSTAYLEFSSLRSEDTAVYYCARLHYFGNNFFFDYWGQGTLVTVSS

SEQ ID NO: 153 - protein, 121 aa, Artificial Sequence
  144D464A HV6d
QVQLVQSGAEVKKPGASVKVSCKASGFTIKDTYIHWVRQAPGQGLEWIGRIDPAIGTTRYDPKFQGRATITTDTSTSTAYMELSSLRSEDTAVYYCARLHYFGNNFFFDYWGQGTLVTVSS

SEQ ID NO: 154 - protein, 121 aa, Artificial Sequence
  144D464A HV6e
QVQLVQSGAEVKKPGASVKVSCKASGFTIKDTYIHWVRQAPGQGLEWMGRIDPAIGTTRYDPKFQGRATITTDTSTSTAYMEFSSLRSEDTAVYYCARLHYFGNNFFFDYWGQGTLVTVSS

SEQ ID NO: 155 - protein, 121 aa, Artificial Sequence
  144D464A HV7a
QVQLVQSGAEVVKPGASVKVSCKASGYNIKDTYIHWVRQAPGQGLEWIGRIDPAIGTTRYDPKFQGRATITADTSTSTAYMEFSSLRSEDTAVYYCARLHYFGNNFFFDYWGQGTLVTVSS

SEQ ID NO: 156 - protein, 121 aa, Artificial Sequence
  144D464A HV7b
QVQLVQSGAEVKKPGASVKVSCKASGFTIKDTYIHWVRQAPGQGLEWIGRIDPAIGTTRYDPKFQGKATITTDTSTSTAYMELSSLRSEDTAVYYCARLHYFGNNFFFDYWGQGTLVTVSS

SEQ ID NO: 157 - protein, 121 aa, Artificial Sequence
  144D464A HV7c
QVQLVQSGAEVKKPGASVKVSCKASGFTIKDTYIHWVRQAPGQGLEWMGRIDPAIGTTRYDPKFQGRATITTDTSTSTAYLEFSSLRSEDTAVYYCARLHYFGNNFFFDYWGQGTLVTVSS

SEQ ID NO: 158 - protein, 121 aa, Artificial Sequence
  144D464A HV8d
QVQLVQSGAEVKKPGASVKVSCKASGYTIKDTYIHWVRQAPGQGLEWMGRIDPAIGTTRYDPKFQGKATITTDTSTDTAYLEFSSLRSEDTAVYYCARLHYFGNNFFFDYWGQGTLVTVSS

SEQ ID NO: 159 - protein, 121 aa, Artificial Sequence
  144D464A HV8e
QVQLVQSGAEVVKPGASVKVSCKASGYTIKDTYIHWVRQAPGQGLEWIGRIDPAIGTTRYDPKFQGRATITADTSTSTVYLEFSSLRSEDTAVYYCARLHYFGNNFFFDYWGQGTLVTVSS

SEQ ID NO: 160 - protein, 121 aa, Artificial Sequence
  144D464A HV10a
QVQLVQSGAEVVKPGASVKVSCKASGFTIKDTYIHWVRQAPGQGLEWIGRIDPAIGTTRYDPKFQGRATITTDTSTDTAYLEFSSLRSEDTAVYYCARLHYFGNNFFFDYWGQGTLVTVSS

SEQ ID NO: 161 - protein, 121 aa, Artificial Sequence
  144D464A HV10b
QVQLVQSGAEVKKPGASVKVSCKASGFNIKDTYIHWVRQAPGQGLEWIGRIDPAIGTTRYDPKFQGRATITTDTSTSTVYLEFSSLRSEDTAVYYCARLHYFGNNFFFDYWGQGTLVTVSS

SEQ ID NO: 162 - protein, 121 aa, Artificial Sequence
  144D464A HV12
QVQLVQSGAEVKKPGASVKLSCKASGFNIKDTYIHWVRQAPGQGLEWIGRIDPAIGTTRYDPKFQGRATITTDTSTSTVYLEFSSLRSEDTAVYYCARLHYFGNNFFFDYWGQGTLLTVSS

SEQ ID NO: 163 - protein, 121 aa, Artificial Sequence
  144D464A HV17
EVQLVQSGAEVVKPGASVKLSCKASGFNIKDTYIHWVKQAPGQGLEWIGRIDPAIGTTRYDPKFQGKATITTDTSTDTVYLEFSSLRSEDTAVYYCARLHYFGNNFFFDYWGQGTLLTVSS

SEQ ID NO: 164 - protein, 109 aa, Artificial Sequence
  144L249B LV0
QAVVTQEPSLTVSPGGTVTLTCRSSTGAVTTSNYANWFQQKPGQAPRTLISGTNDRAPWTPARFSGSLLGGKAALTLSGAQPEDEADYFCALWYSNHLVFGGGTKLTVL

SEQ ID NO: 165 - protein, 121 aa, Artificial Sequence
  144L249B HV0
QVQLVQSGAEVKKPGASVKVSCKASGYTFTDTYIYWVRQAPGQGLEWMGRIDPAIGTTRYDPKFQGRVTITADTSTSTAYMELSSLRSEDTAVYYCARYDYSGSSFYFDYWGQGTLVTVSS

SEQ ID NO: 166 - protein, 109 aa, Artificial Sequence
  144L249B LV7a
QAVVTQEPSLTVSPGGTVTLTCRSSTGAVTTSNYANWVQQKPGQAFTGLIGGTNDRAPGVPARFSGSLLGGKAALTISGAQPEDEADYFCALWYSNHLVFGGGTKLTVL

SEQ ID NO: 167 - protein, 109 aa, Artificial Sequence
  144L249B LV9
QAVVTQEPSLTVSPGGTVTLTCRSSTGAVTTSNYANWVQEKPGQLFTGLIGGTNDRAPGVPARFSGSLLGGKAALTLSGAQPEDEADYFCALWYSNHLVFGGGTKLTVL

SEQ ID NO: 168 - protein, 109 aa, Artificial Sequence
  144L249B LV10
QAVVTQEPSLTVSPGGTVTLTCRSSTGAVTTSNYANWVQEKPGQLFTGLIGGTNDRAPGVPARFSGSLLGGKAALTLSGAQPEDEAIYFCALWYSNHLVFGGGTKLTVL

SEQ ID NO: 169 - protein, 109 aa, Artificial Sequence
  144L249B LV11
QAVVTQEPSLTVSPGGTVTLTCRSSTGAVTTSNYANWVQEKPGQLFTGLIGGTNDRAPGVPARFSGSLLGGKAALTISGAQPEDEAIYFCALWYSNHLVFGGGTKLTVL

SEQ ID NO: 170 - protein, 109 aa, Artificial Sequence
  144L249B LV13
QAVVTQESSLTTSPGGTVTLTCRSSTGAVTTSNYANWVQEKPGQLFTGLIGGTNDRAPGVPARFSGSLLGGKAALTISGAQPEDEAIYFCALWYSNHLVFGGGTKLTVL

SEQ ID NO: 171 - protein, 121 aa, Artificial Sequence
  144L249B HV9a
QVQLVQSGAEVKKPGASVKVSCKASGFNIKDTYIYWVRQAPGQGLEWIGRIDPAIGTTRYDPKFQGKATITTDTSTSTAYLELSSLRSEDTAVYYCARYDYSGSSFYFDYWGQGTLVTVSS

SEQ ID NO: 172 - protein, 121 aa, Artificial Sequence
  144L249B HV9b
QVQLVQSGAEVKKPGASVKVSCKASGFNIKDTYIYWVRQAPGQGLEWIGRIDPAIGTTRYDPKFQGKATITADTSTNTAYLELSSLRSEDTAVYYCARYDYSGSSFYFDYWGQGTLVTVSS

SEQ ID NO: 173 - protein, 121 aa, Artificial Sequence
  144L249B HV10a
QVQLVQSGAEVKKPGASVKVSCKASGFNIKDTYIYWVRQAPGQGLEWIGRIDPAIGTTRYDPKFQGKATITTDTSTNTAYLELSSLRSEDTAVYYCARYDYSGSSFYFDYWGQGTLVTVSS

SEQ ID NO: 174 - protein, 121 aa, Artificial Sequence
  144L249B HV10b
QVQLVQSGAEVKKPGASVKVSCKASGFNIKDTYIYWVRQAPGQGLEWIGRIDPAIGTTRYDPKFQGKATLTTDTSTSTAYLELSSLRSEDTAVYYCARYDYSGSSFYFDYWGQGTLVTVSS

SEQ ID NO: 175 - protein, 121 aa, Artificial Sequence
  144L249B HV10c
QVQLVQSGAEVKKPGASVKVSCKASGFNIKDTYIYWVRQAPGQGLEWIGRIDPAIGTTRYDPKFQGKATLTADTSTNTAYLELSSLRSEDTAVYYCARYDYSGSSFYFDYWGQGTLVTVSS

SEQ ID NO: 176 - protein, 121 aa, Artificial Sequence
  144L249B HV11
QVQLVQSGAEVKKPGASVKVSCKASGFNIKDTYIYWVRQAPGQGLEWIGRIDPAIGTTRYDPKFQGKATLTTDTSTNTAYLELSSLRSEDTAVYYCARYDYSGSSFYFDYWGQGTLVTVSS

SEQ ID NO: 177 - protein, 121 aa, Artificial Sequence
  144L249B HV15
EVQLVQSGAEVVKPGASVKLSCKASGFNIKDTYIYWVKQAPGQGLEWIGRIDPAIGTTRYDPKFQGKATLTADTSTNTAYLELSSLRSEDTAVYYCARYDYSGSSFYFDYWGQGTLLTVSS